(19) United States
(12) Patent Application Publication
(10) Pub. No.: US 2022/0315578 A1
(43) Pub. Date: Oct. 6, 2022

(54) BRD9 BIFUNCTIONAL DEGRADERS AND THEIR METHODS OF USE

(71) Applicant: NOVARTIS AG, Basel (CH)

(72) Inventors: Xin CHEN, Lexington, MA (US); Marie-Line GOUDE, Bartenheim (FR); Edmund Martin HARRINGTON, Plymouth, MA (US); Gregory John HOLLINGWORTH, Sevenoaks (GB); Julien LORBER, Colmar (FR); Martin SENDZIK, Belmont, MA (US); Anna VULPETTI, Basel (CH); Thomas ZOLLER, Andolsheim (FR)

(21) Appl. No.: 17/020,114

(22) Filed: Sep. 14, 2020

Related U.S. Application Data
(60) Provisional application No. 62/900,869, filed on Sep. 16, 2019.
(60) Provisional application No. 62/900,863, filed on Sep. 16, 2019.
(60) Provisional application No. 62/900,865, filed on Sep. 16, 2019.
(60) Provisional application No. 62/900,860, filed on Sep. 16, 2019.

(51) Int. Cl. C07D 471/04; C07D 401/14

(52) U.S. Cl. CPC C07D 471/04; C07D 401/14

(57) ABSTRACT

The disclosure provides BRD9 bifunctional compounds of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), or (BF-V′), or a pharmaceutically acceptable salt, hydrate, solvate, prodrug, stereoisomer, or tautomer thereof, to their preparation, to pharmaceutical compositions comprising them, and to their use in the treatment of diseases and disorders mediated by a bromodomain-containing protein, such as bromodomain-containing protein 9 (BRD9).

### CLAIM OF PRIORITY

This application claims priority from U.S. Application Ser. No. 62/900,860 filed Sep. 16, 2019, 62/900,863 filed Sep. 16, 2019, 62/900,865 filed Sep. 16, 2019, and 62/900,869 filed Sep. 16, 2019, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Sep. 9, 2020, is named PAT058700_SL.txt and is 2,574 bytes in size.

### FIELD OF THE DISCLOSURE

The disclosure provides compounds, their preparation, pharmaceutical compositions comprising them and their use in the treatment of conditions, diseases and disorders mediated by bromodomain-containing protein 9 (BRD9).

### BACKGROUND OF THE DISCLOSURE

Mammalian SWI/SNF (SWItch/Sucrose Non-Fermentable) (mSWI/SNF) is a family of ATP-dependent chromatin remodeling complexes, which regulate chromatin architecture to enable DNA accessibility, insuring timely and appropriate control of gene expression. The bromodomain-containing protein BRD9, which is a subunit of the BAF (SWI/SNF) complex, has emerged as a drug target from genetic screens (CRISPR, shRNA) finding a critical functional dependency in synovial sarcoma and acute myeloid leukemia (AML) while having little or no impact on the majority of other cell lines. (Del Gaudio, N. et al. Cell Death & Disease 10: 338 (2019)).

There is limited understanding of BRD9 function beyond acetyl-lysine recognition based on early chemical probes. To date, the majority of drug discovery efforts have focused on blocking the activity of its bromodomain based on antagonizing the established role of this domain as a histone acetylated lysine reader. Surprisingly, small molecule inhibitors of the BRD9 bromodomain did not reproduce cell-type selective proliferative effects until they were incorporated into molecules containing the Cereblon-binding (CRBN-binding) IMID from thalidomide. (Crawford, T D., et al., Bioorganic & Medicinal Chemistry Letters 27: 3534-3541 (2017); Remillard, D., et al., Angew Chem Int Ed Engl. 56(21): 5738-5743 (2017)).

These bifunctional molecules direct the formation of complexes containing BRD9 and CRBN and leads to the ubiquitination and degradation of BRD9 protein. These observations suggest that BRD9 has an essential scaffolding role beyond its bromodomain reader function and has revived the idea that BRD9 is druggable and as such a valuable target. Therefore, BRD9-directed chemical degraders have the potential to be efficacious in treating a range of BRD9-mediated hematopoietic proliferative disorders, such as cancers.

### SUMMARY OF THE DISCLOSURE

The disclosure provides compounds that recruit a targeted protein, such as a bromodomain-containing protein, e.g., bromodomain-containing protein 9 (BRD9), to E3 Ubiquitin ligase for degradation. In one aspect, the compound or a pharmaceutically acceptable salt, hydrate, solvate, prodrug, stereoisomer, or tautomer thereof, is a compound of Formula (A):

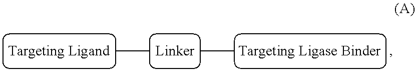

wherein:
the Targeting Ligand is a group that is capable of binding to a bromodomain-containing protein, e.g., BRD9;
the Linker is a group that covalently links the Targeting Ligand to the Targeting Ligase Binder; and
the Targeting Ligase Binder is a group that is capable of binding to a ligase (e.g., Cereblon E3 Ubiquitin ligase).

In another aspect, the disclosure relates to compounds or a pharmaceutically acceptable salt, hydrate, solvate, prodrug, stereoisomer, or tautomer thereof, which function to recruit targeted proteins, e.g., a bromodomain-containing protein, e.g., BRD9, to E3 Ubiquitin ligase for degradation, and methods of preparing and uses thereof.

In another aspect, the disclosure provides a compound of Formula (BF-I):

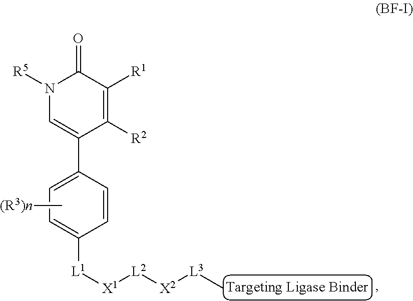

or a pharmaceutically acceptable salt, hydrate, solvate, prodrug, stereoisomer, or tautomer thereof, wherein R¹, R², R³, R⁵, and n are each as defined herein;

-L¹-X¹-L²-X²-L³- denotes the Linker of Formula (L-I) that covalently attaches the Targeting Ligand of Formula (TL-I)

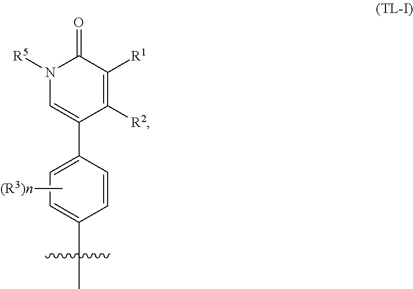

to the Targeting Ligase Binder, wherein L¹, X¹, L², X², and L³ are each as defined herein; and

the Targeting Ligase Binder is a group that is capable of binding to a Ubiquitin ligase, e.g., an E3 Ubiquitin ligase, such as Cereblon.

In another aspect, the disclosure provides a compound of Formula (BF-II):

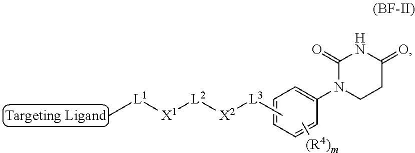

or a pharmaceutically acceptable salt, hydrate, solvate, prodrug, stereoisomer, or tautomer thereof, wherein L¹, X¹, L², X², L³, R⁴, and m are each as defined herein; and

the Targeting Ligand is a group that is capable of binding to a bromodomain-containing protein, e.g., BRD9.

In another aspect, the disclosure provides a compound of Formula (BF-III):

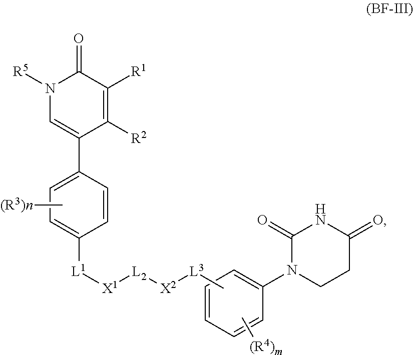

or a pharmaceutically acceptable salt, hydrate, solvate, prodrug, stereoisomer, or tautomer thereof, wherein R¹, R², R³, R⁴, R⁵, n, m, L¹, X¹, L², X², and L³ are each as defined herein.

In another aspect, the disclosure provides a compound of Formula (BF-I′):

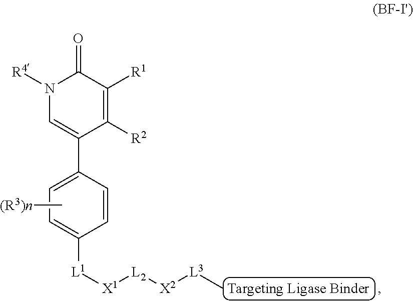

or a pharmaceutically acceptable salt, hydrate, solvate, prodrug, stereoisomer, or tautomer thereof, wherein R¹, R², R³, R^{4′}, and n are each as defined herein;

-L¹-X¹-L²-X²-L³- denotes the Linker of Formula (L-I) that covalently attaches the Targeting Ligand of Formula (TL-I′)

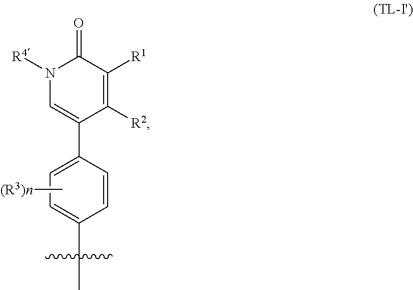

to the Targeting Ligase Binder, wherein L¹, X¹, L², X², and L³ are each as defined herein; and

the Targeting Ligase Binder is a group that is capable of binding to a ubiquitin ligase, e.g., an E3 Ubiquitin ligase, such as Cereblon.

In another aspect, the disclosure provides a compound of Formula (BF-II′):

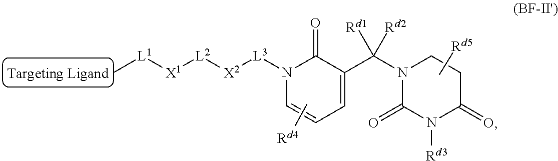

or a pharmaceutically acceptable salt, hydrate, solvate, prodrug, stereoisomer, or tautomer thereof, wherein L¹, X¹, L², X², L³, R^{d1}, R^{d2}, R^{d3}, R^{d4}, and R^{d5} are each as defined herein; and

the Targeting Ligand is a group that is capable of binding to a bromodomain-containing protein, e.g., BRD9.

In another aspect, the disclosure provides a compound of Formula (BF-III′):

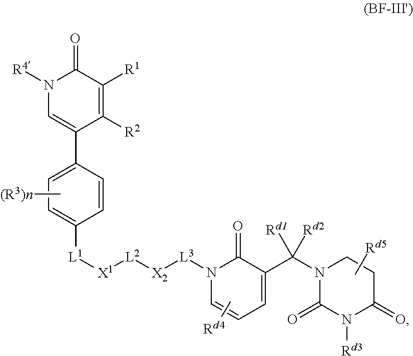

or a pharmaceutically acceptable salt, hydrate, solvate, prodrug, stereoisomer, or tautomer thereof, wherein R¹, R², R³, R⁴, n, L¹, X¹, L², X², L³, R^{d1}, R^{d2}, R^{d3}, R^{d4}, and R^{d5} are each as defined herein.

In another aspect, the disclosure provides a compound of Formula (BF-IV′):

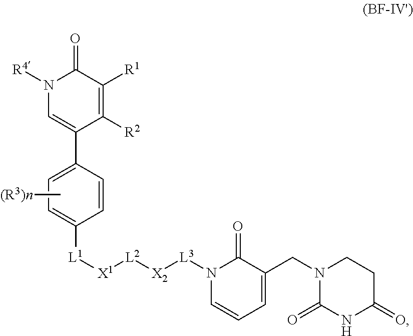

or a pharmaceutically acceptable salt, hydrate, solvate, prodrug, stereoisomer, or tautomer thereof, wherein R¹, R², R³, R^{4′}, n, L¹, X¹, L², X², and L³ are each as defined herein. In another aspect, the disclosure provides a Linker of Formula (L-I):

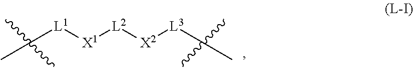

or a pharmaceutically acceptable salt, hydrate, solvate, prodrug, stereoisomer or tautomer thereof, wherein L¹, X¹, L², X², and L³ are each defined herein, the Linker is covalently attached to the Targeting Ligand via L¹, and to the Targeting Ligase Binder via L³.

In another aspect, the disclosure provides a pharmaceutical composition comprising a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof, and a pharmaceutically acceptable carrier.

In another aspect, the disclosure provides a pharmaceutical combination comprising a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof, and a therapeutic agent.

In another aspect, the disclosure provides a method of inhibiting or modulating a bromodomain-containing protein 9 (BRD9) in a subject in need thereof, the method comprising administering to the subject a therapeutically effective amount of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof.

In another aspect, the disclosure provides a method of treating or preventing a disorder in a subject in need thereof, the method comprising administering to the subject a therapeutically effective amount of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof.

In another aspect, the disclosure provides a method of treating or preventing a disorder mediated by a bromodomain protein, e.g., BRD9, in a subject in need thereof, the method comprising administering to the subject a therapeutically effective amount of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof.

In another aspect, the disclosure provides a method of treating cancer in a subject in need thereof, the method comprising administering to the subject a therapeutically effective amount of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof.

Unless otherwise defined, all technical and scientific terms used herein have the same meaning as commonly understood by one of ordinary skill in the art to which this disclosure belongs. In the specification and claims, the singular forms also include the plural unless the context clearly dictates otherwise. Although methods and materials similar or equivalent to those described herein can be used in the practice or testing of the present invention, suitable methods and materials are described below. All publications, patent applications, patents, and other references mentioned herein are incorporated by reference in their entireties for all purposes. The references cited herein are not admitted to be prior art to the claimed invention.

In the case of conflict, the present specification, including definitions, will control. In addition, the materials, methods, and examples are illustrative only and are not intended to be limiting.

Other features and advantages of compounds, compositions, and methods disclosed herein will be apparent from the following detailed description and claims.

### BRIEF DESCRIPTION OF THE DRAWINGS

FIG. 1 depicts a schematic of a bifunctional compound, such as a compound disclosed herein, which is bound to a protein of interest (POI), and which has recruited the POI to the E3 Ubiquitin ligase binding complex for tagging with Ubiquitin (Ub), marking the POI for degradation by the ligase (e.g., Cereblon E3 Ubiquitin ligase).

### DETAILED DESCRIPTION

The disclosure provides compounds or a pharmaceutically acceptable salt, hydrate, solvate, prodrug, stereoisomer, or tautomer thereof, that function to recruit targeted proteins to E3 Ubiquitin ligase for degradation, and methods of preparation and uses thereof.

In one aspect, the disclosure provides compounds or a pharmaceutically acceptable salt, hydrate, solvate, prodrug, stereoisomer, or tautomer thereof, which recruit a targeted protein, such as a bromodomain-containing protein, e.g., bromodomain-containing protein 9 (BRD9), to E3 Ubiquitin ligase for degradation. In an embodiment, the compound or a pharmaceutically acceptable salt, hydrate, solvate, prodrug, stereoisomer, or tautomer thereof, is a compound of Formula (A):

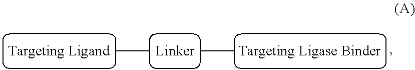

wherein:
the Targeting Ligand is a group that is capable of binding to a bromodomain-containing protein, e.g., BRD9;
the Linker is a group that covalently links the Targeting Ligand to the Targeting Ligase Binder; and
the Targeting Ligase Binder is a group that is capable of binding to a ligase (e.g., Cereblon E3 Ubiquitin ligase).

### Targeting Ligand

The Targeting Ligand is a small molecule moiety that is capable of binding to a protein of interest (POI), such as a bromodomain-containing protein, e.g., BRD9.

In an embodiment, the Targeting Ligand is a compound of Formula (TL-I):

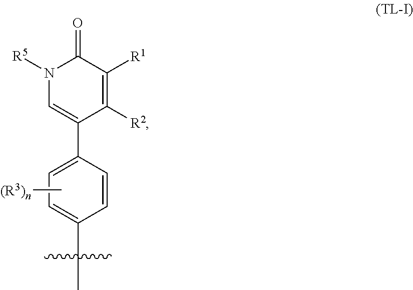

or a pharmaceutically acceptable salt, hydrate, solvate, prodrug, stereoisomer, or tautomer thereof, wherein:

R¹ and R² are independently selected from the group consisting of hydrogen and C₁₋₆ alkyl; or R¹ and R² together with the atoms to which they are attached form an aryl or heteroaryl;

R³ are each independently selected from the group consisting of C₁₋₆ alkyl, C₁₋₆ alkoxyl, and halogen;

R⁵ is selected from the group consisting of hydrogen and C₁₋₆ alkyl;

n is 0, 1, or 2.

In an embodiment, the Targeting Ligand is a compound of Formula (TL-I′):

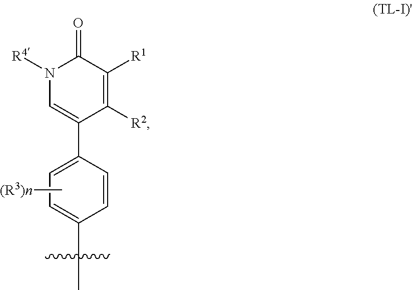

or a pharmaceutically acceptable salt, hydrate, solvate, prodrug, stereoisomer, or tautomer thereof, wherein:

R¹ and R² are independently selected from the group consisting of hydrogen and C₁₋₆ alkyl; or R¹ and R² together with the atoms to which they are attached form an aryl or heteroaryl;

R³ are each independently selected from the group consisting of C₁₋₆ alkyl, C₁₋₆ alkoxyl, and halogen;

R^{4′} is selected from the group consisting of hydrogen and C₁₋₆ alkyl; and

n is 0, 1, or 2.

In an embodiment, R¹ and R² together with the atoms to which they are attached form an aryl or heteroaryl. In an embodiment, R¹ and R² together with the atoms to which they are attached form a phenyl. In an embodiment, R¹ and R² together with the atoms to which they are attached form a heteroaryl. In an embodiment, R¹ and R² together with the atoms to which they are attached form a 5- or 6-membered heteroaryl. In an embodiment, R¹ and R² together with the atoms to which they are attached form a 6-membered heteroaryl. In an embodiment, R¹ and R² together with the atoms to which they are attached form a 6-membered nitrogen-containing heteroaryl. In an embodiment, R¹ and R² together with the atoms to which they are attached form a pyridyl.

In an embodiment, the Targeting Ligand has Formula (TL-II):

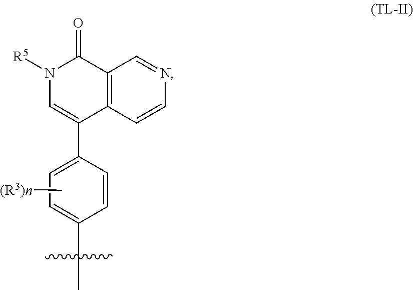

or a pharmaceutically acceptable salt, hydrate, solvate, prodrug, stereoisomer, or tautomer thereof.

In an embodiment of the Targeting Ligand, R³ is selected from the group consisting of methoxyl, chloro, and fluoro. In an embodiment, R³ is methoxyl. In an embodiment, R³ is chloro or fluoro.

In an embodiment, n is 1 or 2.

In an embodiment, R³ is methoxyl and n is 1 or 2. In an embodiment, n is 1. In an embodiment, n is 2. In an embodiment, n is 0.

In an embodiment of Formula (TL-I) or (TL-II), R⁵ is hydrogen or methyl. In an embodiment, R⁵ is methyl. In an embodiment, R⁵ is C₂₋₆ alkyl. In an embodiment, R⁵ is n-butyl.

In an embodiment, the Targeting Ligand has Formula (TL-II′):

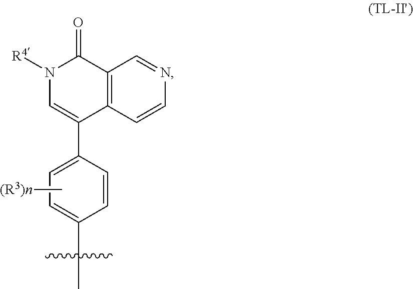

or a pharmaceutically acceptable salt, hydrate, solvate, prodrug, stereoisomer, or tautomer thereof.

In an embodiment of Formula (TL-I′) or (TL-II′), R^{4′} is hydrogen or methyl. In an embodiment, R^{4′} is methyl. In an embodiment, R^{4′} is C₂₋₆ alkyl. In an embodiment, R^{4′} is n-butyl.

In an embodiment the Targeting Ligand has Formula (TL-III):

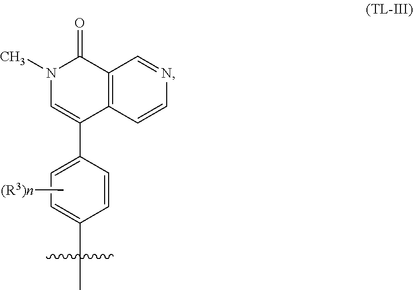

or a pharmaceutically acceptable salt, hydrate, solvate, prodrug, stereoisomer, or tautomer thereof.

In an embodiment, the Targeting Ligand is selected from the group consisting of:

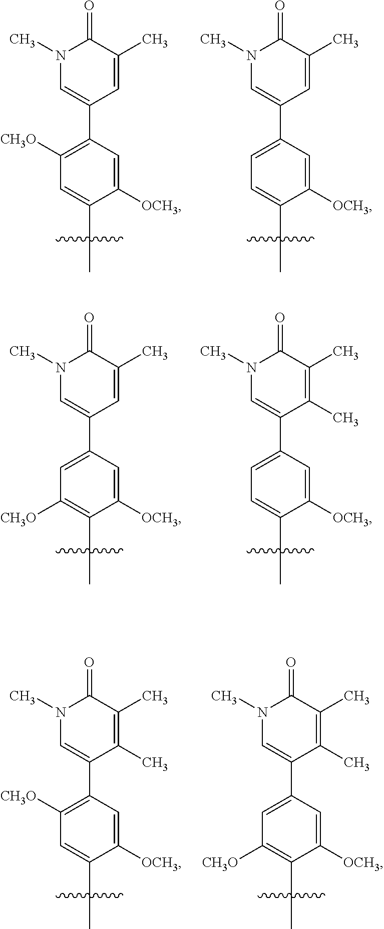

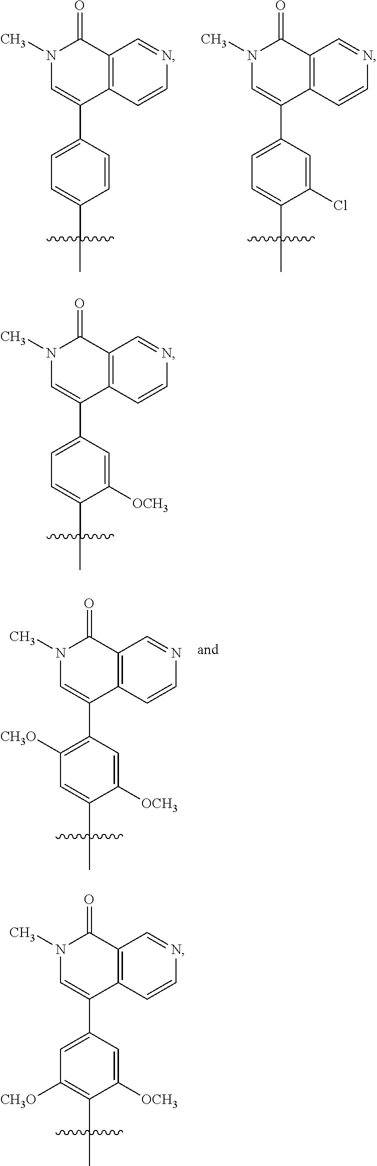

or a pharmaceutically acceptable salt, hydrate, solvate, prodrug, stereoisomer, or tautomer thereof.

In an embodiment, the Targeting Ligand is selected from the group consisting of:

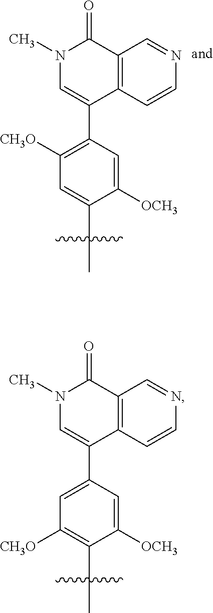

or a pharmaceutically acceptable salt, hydrate, solvate, prodrug, stereoisomer, or tautomer thereof.

In an embodiment, the Targeting Ligand has Formula (TL-III′):

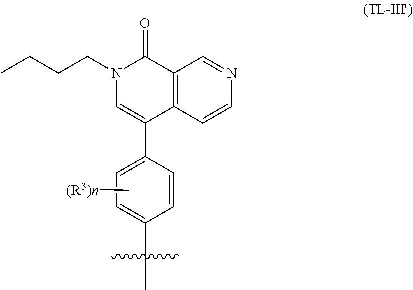

or a pharmaceutically acceptable salt, hydrate, solvate, prodrug, stereoisomer, or tautomer thereof.

In an embodiment, the Targeting Ligand is selected from the group consisting of:

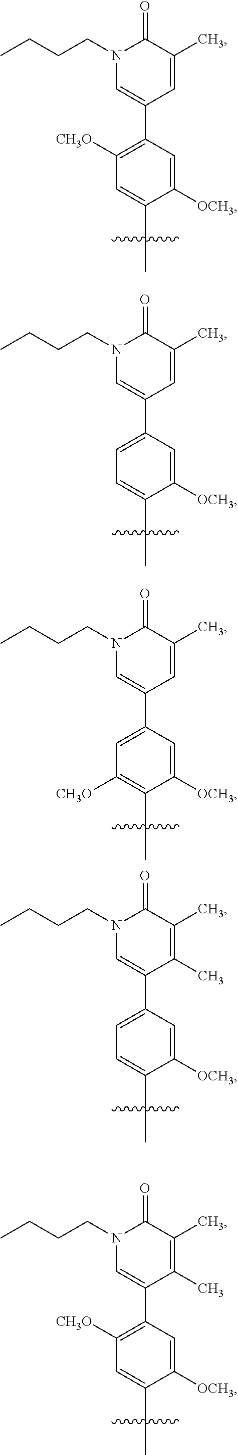

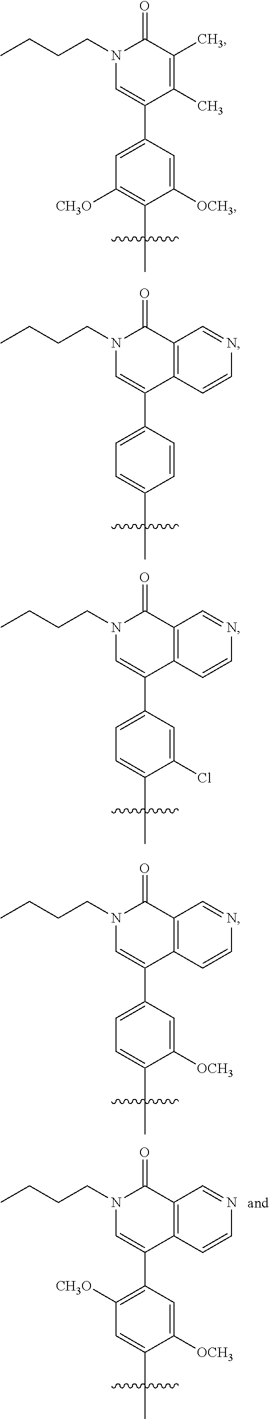

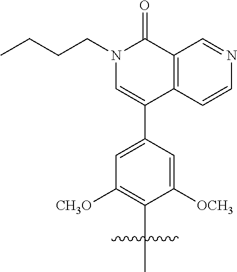

or a pharmaceutically acceptable salt, hydrate, solvate, prodrug, stereoisomer, or tautomer thereof.

In an embodiment, the Targeting Ligand is selected from the group consisting of:

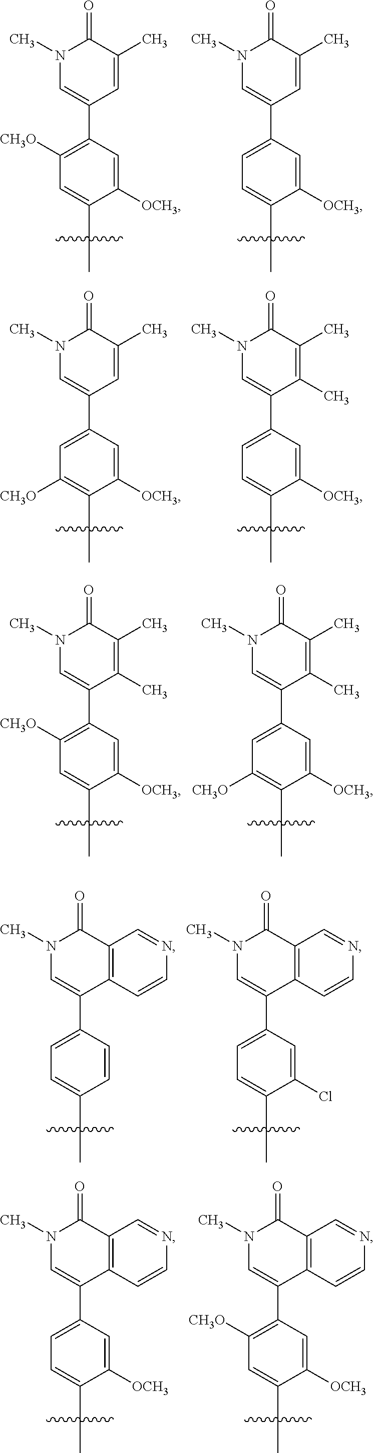

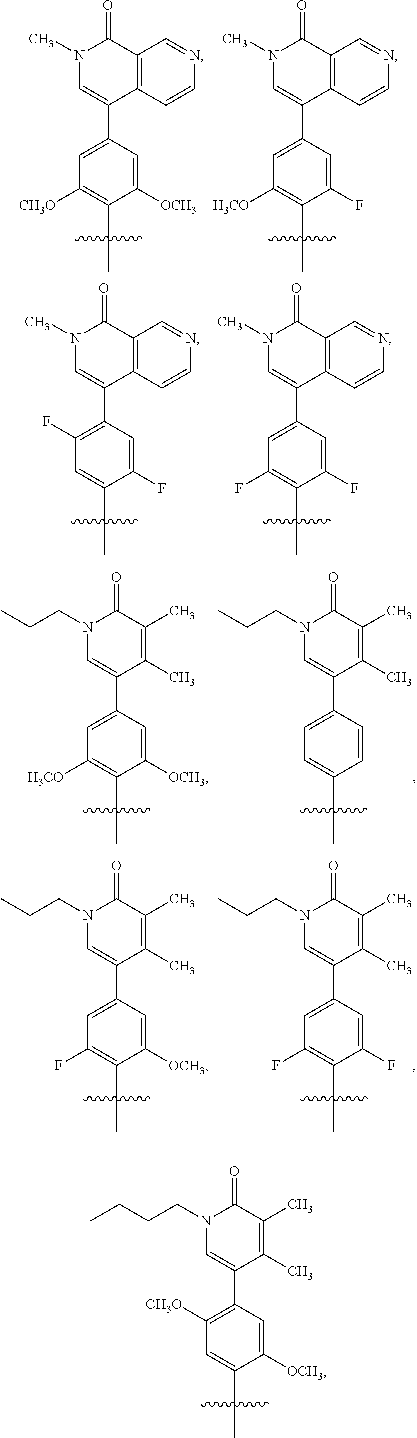

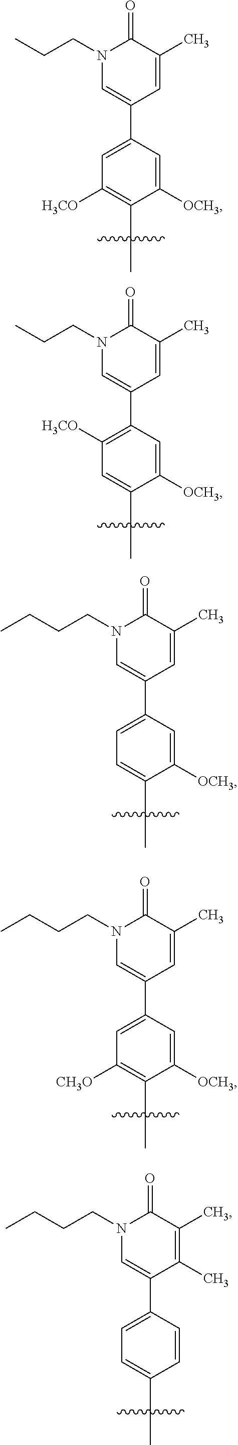

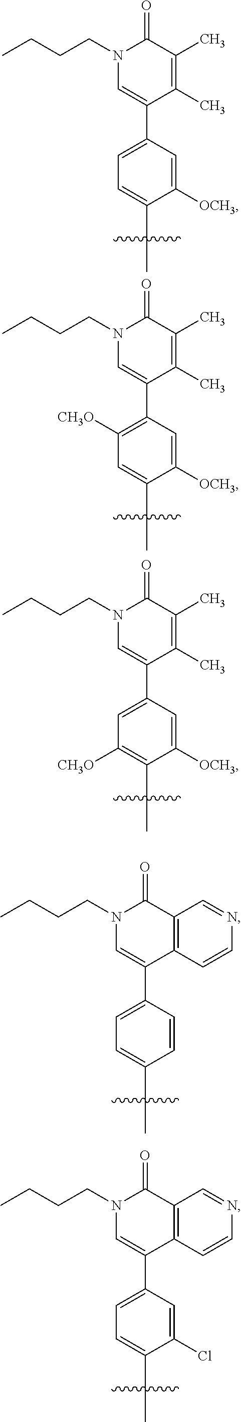

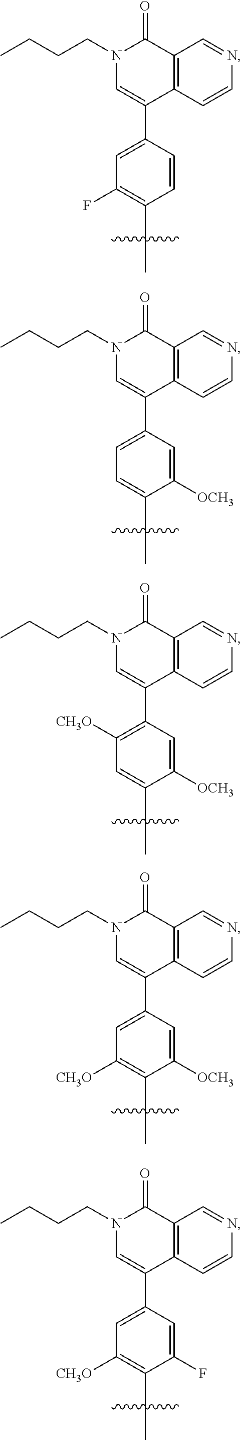

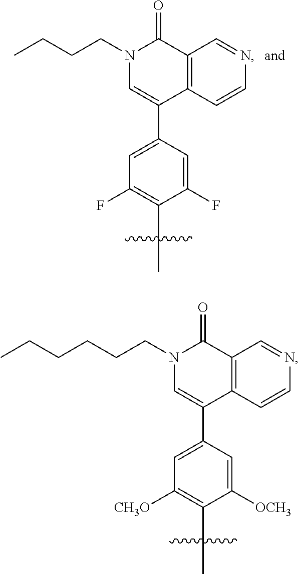

or a pharmaceutically acceptable salt, hydrate, solvate, prodrug, stereoisomer, or tautomer thereof.

### Targeting Ligase Binder

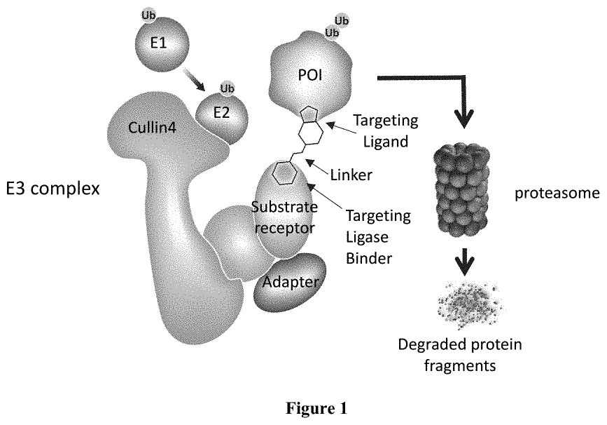

The Targeting Ligase Binder brings a protein of interest (POI) into close proximity to a Ubiquitin ligase for tagging with Ubiquitin (Ub), marking the POI for degradation by the ligase, through the linking of the Target Ligase Binder bound to the Ubiquitin ligase (e.g., an E3 Ubiquitin ligase binding complex), Linker (L), and a Targeting Ligand (TL) bound to the POI. See, e.g., FIG. 1.

In an embodiment, the Targeting Ligase Binder is a compound of Formula (TLB-I):

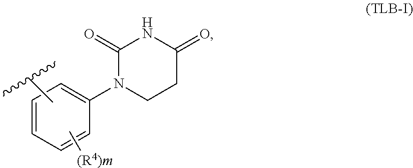

or a pharmaceutically acceptable salt, hydrate, solvate, prodrug, stereoisomer, or tautomer thereof, wherein:

R⁴ is selected from the group consisting of OH, C₁₋₆ alkyl, C₁₋₆ alkoxyl, and halogen; and

m is 0, 1, or 2.

In an embodiment, R⁴ is halogen, e.g., chloro or fluoro. In an embodiment, R⁴ is C₁₋₆ alkyl, e.g., methyl. In an embodiment, R⁴ is C₁₋₆ alkoxyl, e.g., methoxyl. In an embodiment, R⁴ is OH. In an embodiment, m is 0. In an embodiment, m is 1. In an embodiment, m is 2. In an embodiment, R⁴ is halogen, e.g., chloro or fluoro, and m is 1. In an embodiment, R⁴ is C₁₋₆ alkyl, e.g., methyl, and m is 1. In an embodiment, R⁴ is C₁₋₆ alkoxyl, e.g., methoxyl, and m is 1. In an embodiment, R⁴ is OH, and m is 1.

In an embodiment, the Targeting Ligase Binder is a compound of Formula (TLB-II):

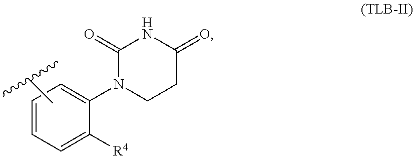

or a pharmaceutically acceptable salt, hydrate, solvate, prodrug, stereoisomer, or tautomer thereof.

In an embodiment, R⁴ is halogen, e.g., chloro or fluoro. In an embodiment, R⁴ is C₁₋₆ alkyl, e.g., methyl. In an embodiment, R⁴ is C₁₋₆ alkoxyl, e.g., methoxyl.

In an embodiment, the Targeting Ligase Binder is a compound of Formula (TLB-III):

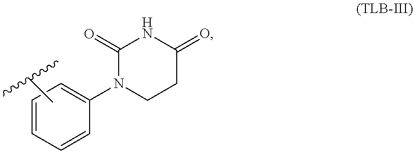

or a pharmaceutically acceptable salt, hydrate, solvate, prodrug, stereoisomer, or tautomer thereof.

In an embodiment, the Targeting Ligase Binder is a compound of Formula (TLB-IIIa):

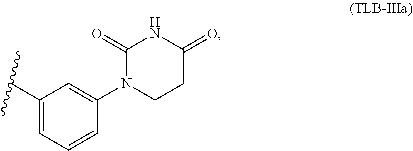 [SMILES: *c1cccc(N2CCC(=O)NC2=O)c1]

or a pharmaceutically acceptable salt, hydrate, solvate, prodrug, stereoisomer, or tautomer thereof.

In an embodiment, the Targeting Ligase Binder is a compound of Formula (TLB-IIIb):

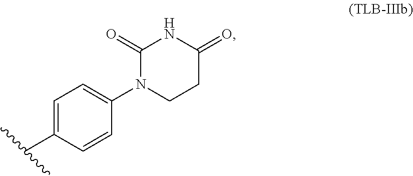 [SMILES: *c1ccc(N2CCC(=O)NC2=O)cc1]

or a pharmaceutically acceptable salt, hydrate, solvate, prodrug, stereoisomer, or tautomer thereof.

In an embodiment, the Targeting Ligase Binder is a compound of Formula (TLB-IIIc):

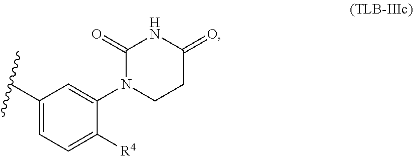

or a pharmaceutically acceptable salt, hydrate, solvate, prodrug, stereoisomer, or tautomer thereof. In an embodiment, R⁴ is halogen, e.g., chloro or fluoro. In an embodiment, R⁴ is C₁₋₆ alkyl, e.g., methyl. In an embodiment, R⁴ is C₁₋₆ alkoxyl, e.g., methoxyl.

In an embodiment, the Targeting Ligase Binder is a compound of Formula (TLB-I′):

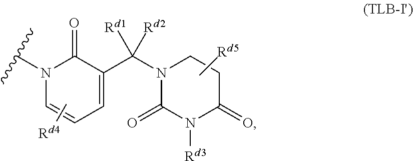

or a pharmaceutically acceptable salt, hydrate, solvate, prodrug, stereoisomer, or tautomer thereof, wherein R^{d1} and R^{d2} are each independently selected from the group consisting of H, C₁₋₆ alkyl, C₁₋₆ alkoxyl, C₁₋₆ haloalkyl, and C₁₋₆ heteroalkyl; R^{d3} is H; R^{d4} is selected from the group consisting of H, C₁₋₆ alkyl, halo, C₁₋₆ haloalkyl, and C₁₋₆ heteroalkyl; and R^{d5} is selected from the group consisting of H, C₁₋₆ alkyl, halo, C₁₋₆ haloalkyl, and C₁₋₆ heteroalkyl.

In an embodiment, R^{d1} and R^{d2} are both methyl. In an embodiment, R^{d1} and R^{d2} are both H. In an embodiment, R^{d4} is H or C₁₋₆ alkyl, e.g., methyl. In an embodiment, R^{d5} is H or C₁₋₆ alkyl, e.g., methyl.

In an embodiment, the Targeting Ligase Binder is a compound of Formula (TLB-II′):

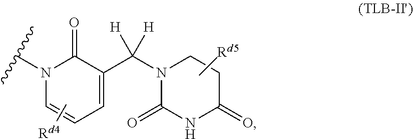

or a pharmaceutically acceptable salt, hydrate, solvate, prodrug, stereoisomer, or tautomer thereof. In an embodiment, R^{d4} is H or C₁₋₆ alkyl, e.g., methyl. In an embodiment, R^{d5} is H or C₁₋₆ alkyl, e.g., methyl.

In an embodiment, the Targeting Ligase Binder is a compound of Formula (TLB-III′):

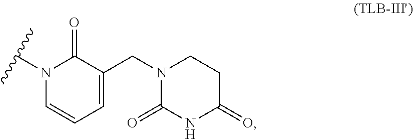 [SMILES: *n1cccc(CN2CCC(=O)NC2=O)c1=O]

or a pharmaceutically acceptable salt, hydrate, solvate, prodrug, stereoisomer, or tautomer thereof.

### Linker

In another aspect, the Linker is a moiety that covalently links, i.e., attaches or connects, the Targeting Ligand to the Targeting Ligase Binder.

In an embodiment, the Linker is a moiety that covalently links, i.e., attaches or connects, the Targeting Ligand to the Targeting Ligase Binder.

In an embodiment, the Linker is a compound of Formula (L-I):

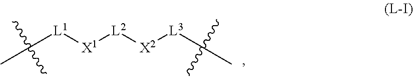

or a pharmaceutically acceptable salt, hydrate, solvate, prodrug, stereoisomer, or tautomer thereof, wherein:

L¹ is selected from the group consisting of a bond, O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the Targeting Ligand;

X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;

L² is selected from the group consisting of a bond, O, NR′, C₁₋₆ alkylene, and C₁₋₆ heteroalkylene; or

X¹-L²-X² form a spiroheterocyclyl;

L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O , wherein * denotes the point of attachment of L³ to X² in Formula (L-I); wherein no more than 2 of L¹, X¹, X², L², and L³ can simultaneously be a bond; and

R′ is selected from the group consisting of hydrogen and C₁₋₆ alkyl.

In an embodiment, L¹ is —O—, C₁₋₆ alkylene, e.g., —CH₂- or —CH₂CH₂—, or C₁₋₆ heteroalkylene, e.g.,—O-CH₂CH₂—. In an embodiment, L¹ is —O— or C₁₋₆ alkylene. In an embodiment, L¹ is C(O).

In an embodiment, one of X¹ and X² is not a bond. In an embodiment, one of X¹ and X² is a bond and the other is a carbocyclyl or heterocyclyl wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen. In an embodiment, one of X¹ and X² is a bond and the other is a heterocyclyl wherein the heterocyclyl is substituted with 0-4 occurrences of R^{a}, wherein each R^{a} is independently selected from the group consisting of C₁₋₆ alkyl, C₁₋₆ alkoxyl, and halogen.

In an embodiment, X¹ and X² are each independently selected from the group consisting of cyclohexyl, piperidinyl, and piperazinyl, wherein the cyclohexyl, piperidinyl, and piperazinyl are substituted with 0-4 occurrences of R^{a}, wherein each R^{a} is independently selected from the group consisting of C₁₋₆ alkyl, C₁₋₆ alkoxyl, and halogen.

In an embodiment, —X¹-L²-X²— is selected from the group consisting of

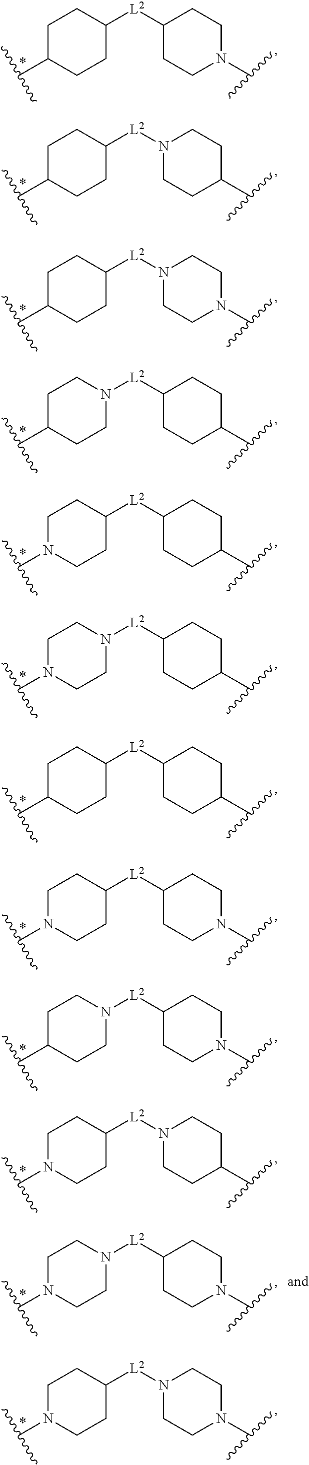

wherein the cyclohexyl, piperidinyl, and piperazinyl are substituted with 0-4 occurrences of R^{a}, wherein each R^{a} is independently selected from the group consisting of C₁₋₆ alkyl, C₁₋₆ alkoxyl, and halogen, or a pharmaceutically acceptable salt, hydrate, solvate, prodrug, stereoisomer, or tautomer thereof, wherein * denotes the point of attachment to L¹.

In an embodiment, X¹ and X² are each independently selected from the group consisting of piperidinyl and piperazinyl, wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen. In an embodiment, X¹ and X² are both piperidinyl, wherein each piperidinyl is substituted with 0-4 occurrences of R^{a}, wherein each R^{a} is independently selected from the group consisting of C₁₋₆ alkyl, C₁₋₆ alkoxyl, and halogen.

In an embodiment, —X¹-L²-X²— is selected from the group consisting of

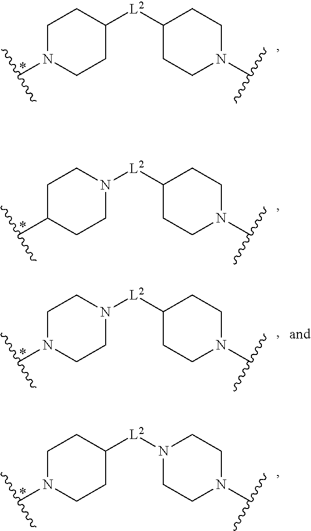

wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen, or a pharmaceutically acceptable salt, hydrate, solvate, prodrug, stereoisomer, or tautomer thereof, wherein * denotes the point of attachment to L¹. In an embodiment, —X¹-L²-X²— is

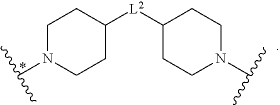

In an embodiment, L² is selected from the group consisting of O, C₁₋₆ alkylene, and C₁₋₆ heteroalkylene. In an embodiment, L² is —CH₂—, O, or C₁₋₃ heteroalkylene. In an embodiment, L² is oxygen. In an embodiment, L₂ is —CH₂—.

In an embodiment, each R^{a} is halogen. In an embodiment, each R^{a} is fluoro.

In an embodiment, —X¹-L²-X²— forms a spiroheterocyclyl having the structure

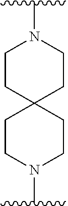 [SMILES: *N1CCC2(CC1)CCN(*)CC2]

substituted with 0-4 occurrences of R^{b}, wherein each R^{b} is independently selected from the group consisting of C₁₋₆ alkyl, C₁₋₆ alkoxyl, and C₁₋₆ hydroxyalkyl.

In an embodiment, —X¹-L²-X²— forms a spiroheterocyclyl having the structure

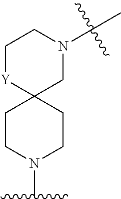

substituted with 0-4 occurrences of R^{c}, wherein Y is selected from the group consisting of CH₂, oxygen, and —NR; and each R^{c} is independently selected from the group consisting of C₁₋₆ alkyl, C₁₋₆ alkoxyl, and C₁₋₆ hydroxyalkyl. In an embodiment, Y is CH₂, CH(C₁₋₃ alkyl), C(C₁₋₃ alkyl)₂, oxygen, NH, or N(C₁₋₃ alkyl).

In an embodiment, L³ is selected from the group consisting of O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O , wherein * denotes the point of attachment of L³ to X². In an embodiment, L³ is selected from the group consisting of O, C(O), C₁₋₆ alkylene, C₁₋₆ heteroalkylene, and *C(O)—C₁₋₆ alkylene-O. In an embodiment, L³ is selected from the group consisting of O, C(O), C₁₋₃ alkylene, C₁₋₃ heteroalkylene, and *C(O)—C₁₋₃ alkylene-O . In an embodiment, L³ is selected from the group consisting of bond, C₁₋₆ alkylene, C₁₋₆ heteroalkylene, *C(O)—C₁₋₆ alkylene, and *C(O)—C₁₋₆ heteroalkylene. In an embodiment, L³ is selected from the group consisting of C₁₋₆ alkylene, C₁₋₆ heteroalkylene, *C(O)—C₁₋₆ alkylene, and *C(O)—C₁₋₆ heteroalkylene.

In an embodiment, the Linker is a compound having the following formula

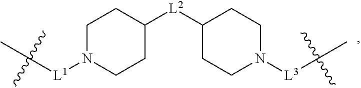

wherein each piperidinyl is substituted with 0-4 occurrences of R^{a}, wherein each R^{a} is independently selected from the group consisting of C₁₋₆ alkyl, C₁₋₆ alkoxyl, and halogen. In an embodiment, L¹ and L³ are each independently C₁₋₆ alkylene. In an embodiment, L¹ and L³ are each methylene. In an embodiment, L¹ and L³ are each ethylene. In an embodiment, L¹ is methylene and L³ is ethylene. In an embodiment, L² is —CH₂—, O, or C₁₋₃ heteroalkylene. In an embodiment, L² is oxygen. In an embodiment, L² is —CH₂—. In an embodiment, L² is oxygen. In an embodiment, each R^{a} is halogen. In an embodiment, each R^{a} is fluoro.

In an embodiment, the Linker is selected from the group consisting of:

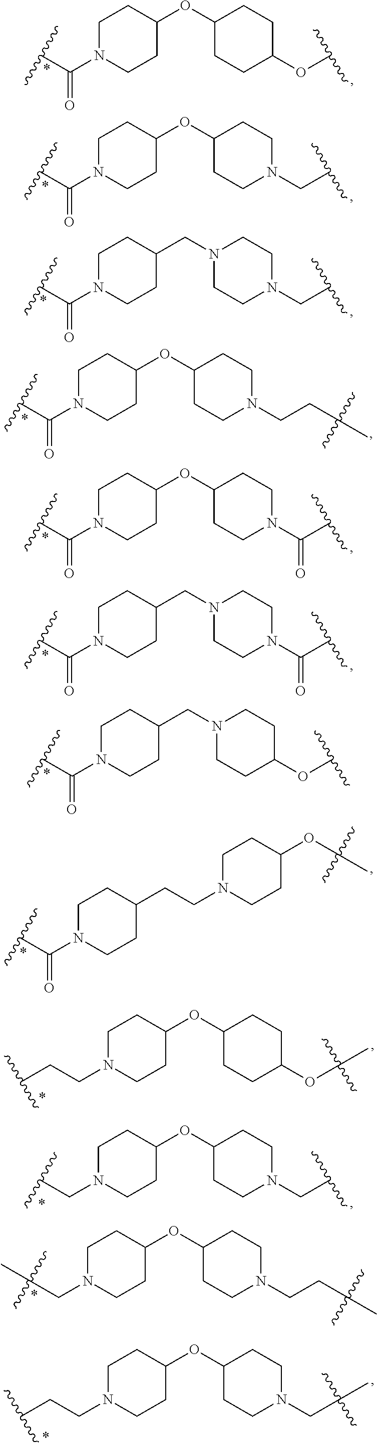

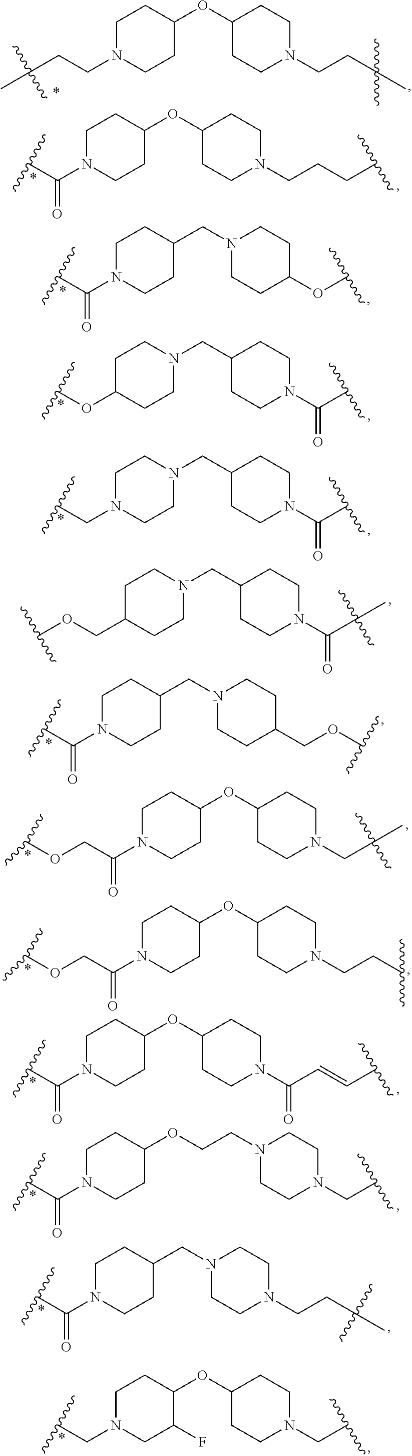

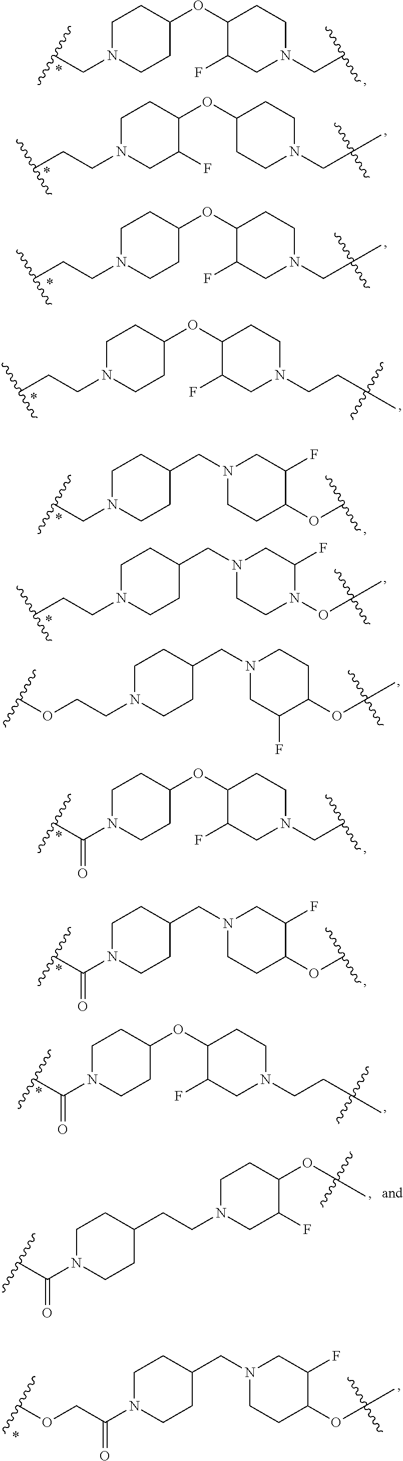

or a pharmaceutically acceptable salt, hydrate, solvate, prodrug, stereoisomer, or tautomer thereof, wherein * denotes the point of attachment to the Targeting Ligase Binder.

In an embodiment, the Linker is selected from the group consisting of:

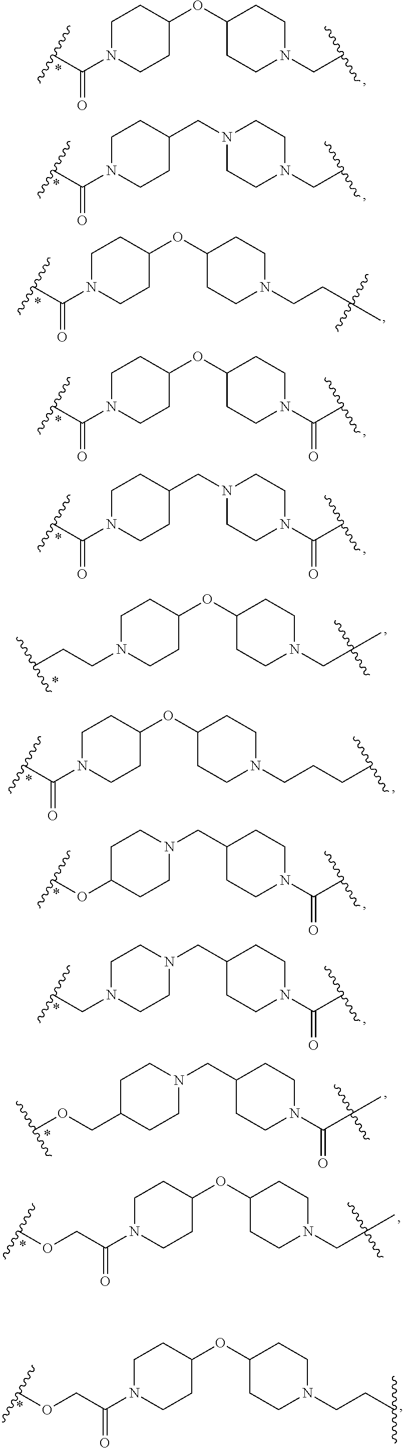

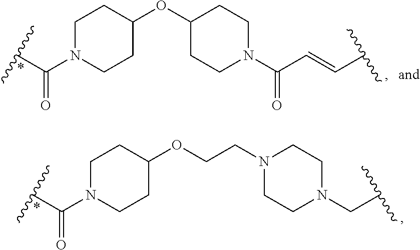

or a pharmaceutically acceptable salt, hydrate, solvate, prodrug, stereoisomer, or tautomer thereof, wherein * denotes the point of attachment to the Targeting Ligase Binder.

In an embodiment, the Linker is selected from the group consisting of:

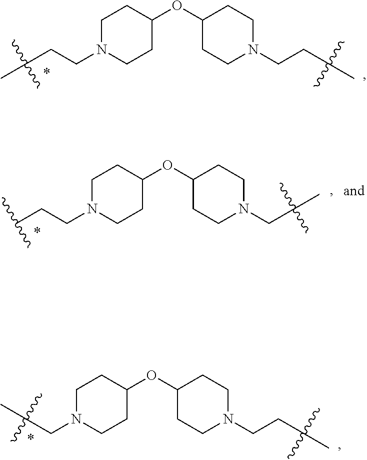 [SMILES: *CCN1CCC(OC2CCN(CC*)CC2)CC1 ; *CCN1CCC(OC2CCN(C*)CC2)CC1 ; *CCN1CCC(OC2CCN(C*)CC2)CC1]

or a pharmaceutically acceptable salt, hydrate, solvate, prodrug, stereoisomer, or tautomer thereof, wherein * denotes the point of attachment to the Targeting Ligase Binder.

### Structures of Targeting Ligase Binder-Linkers

In another aspect, the Targeting Ligase Binder-Linker has Formula (TLBL-I):

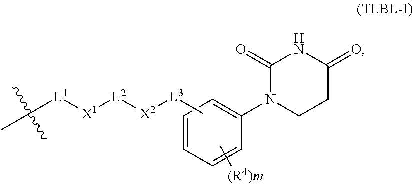

or a pharmaceutically acceptable salt, hydrate, solvate, prodrug, stereoisomer, or tautomer thereof, wherein:

L¹ is selected from the group consisting of a bond, O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the Targeting Ligand;

X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;

L² is selected from the group consisting of a bond, O, NR′, C₁₋₆ alkylene, and C₁₋₆ heteroalkylene; or

X¹-L²-X² form a spiroheterocyclyl;

L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O—, wherein * denotes the point of attachment of L³ to X² in Formula (TLBL-I); wherein no more than 2 of L¹, X¹, X², L², and L³ can simultaneously be a bond; and wherein the point of attachment to the Targeting Ligand is through L¹.

In another aspect, the Targeting Ligase Binder-Linker has Formula (TLBL-I′):

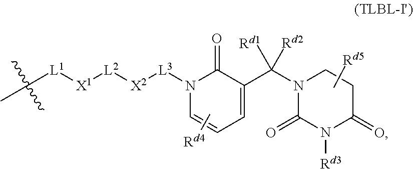

or a pharmaceutically acceptable salt, hydrate, solvate, prodrug, stereoisomer, or tautomer thereof, wherein:

L¹ is selected from the group consisting of a bond, O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the Targeting Ligand;

X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;

L² is selected from the group consisting of a bond, O, NR′, C₁₋₆ alkylene, and C₁₋₆ heteroalkylene; or

X¹-L²-X² form a spiroheterocyclyl;

L³ is selected from the group consisting of a bond, C₁₋₆ alkylene, C₁₋₆ heteroalkylene, *C(O)—C₁₋₆ alkylene, and *C(O)—C₁₋₆ heteroalkylene, wherein * denotes the point of attachment of L³ to X² in Formula (TLBL-I′); wherein no more than 2 of L¹, X¹, X², L², and L³ can simultaneously be a bond;

R′ is selected from the group consisting of hydrogen and C₁₋₆ alkyl;

R^{d1} and R^{d2} are each independently selected from the group consisting of H, C₁₋₆ alkyl, C₁₋₆ alkoxyl, C₁₋₆ haloalkyl, and C₁₋₆ heteroalkyl;

R^{d3} is H;

R^{d4} is selected from the group consisting of H, C₁₋₆ alkyl, halo, C₁₋₆ haloalkyl, and C₁₋₆ heteroalkyl; and

R^{d5} is selected from the group consisting of H, C₁₋₆ alkyl, halo, C₁₋₆ haloalkyl, and C₁₋₆ heteroalkyl; and wherein the point of attachment to the Targeting Ligand is through L¹.

In an embodiment of Targeting Ligase Binder-Linker, L¹ is —O—, C₁₋₆ alkylene, or C₁₋₆ heteroalkylene. In an embodiment, L¹ is —O— or C₁₋₆ alkylene. In an embodiment, L¹ is C(O).

In an embodiment, one of X¹ and X² is not a bond. In an embodiment, one of X¹ and X² is a bond and the other is a carbocyclyl or heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen. In an embodiment, one of X¹ and X² is a bond and the other is a heterocyclyl, wherein the heterocyclyl is substituted with 0-4 occurrences of R^{a}, wherein each R^{a} is independently selected from the group consisting of C₁₋₆ alkyl, C₁₋₆ alkoxyl, and halogen.

In an embodiment, X¹ and X² are each independently selected from the group consisting of cyclohexyl, piperidinyl, and piperazinyl, wherein each cyclohexyl, piperidinyl, and piperazinyl is substituted with 0-4 occurrences of R^{a}, wherein each R^{a} is independently selected from the group consisting of C₁₋₆ alkyl, C₁₋₆ alkoxyl, and halogen.

In an embodiment, —X¹-L²-X²— is selected from the group consisting of

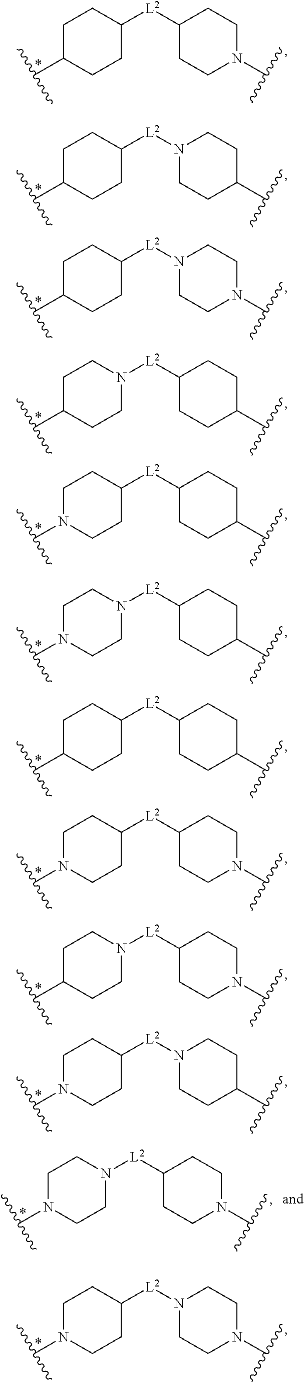

wherein each cyclohexyl, piperidinyl, and piperazinyl is substituted with 0-4 occurrences of R^{a}, wherein each R^{a} is independently selected from the group consisting of C₁₋₆ alkyl, C₁₋₆ alkoxyl, and halogen, or a pharmaceutically acceptable salt, hydrate, solvate, prodrug, stereoisomer, or tautomer thereof, wherein * denotes the point of attachment to L¹.

In an embodiment, X¹ and X² are each independently selected from the group consisting of piperidinyl and piperazinyl, wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen. In an embodiment, X¹ and X² are both piperidinyl, wherein each piperidinyl is substituted with 0-4 occurrences of R^{a}, wherein each R^{a} is independently selected from the group consisting of C₁₋₆ alkyl, C₁₋₆ alkoxyl, and halogen.

In an embodiment, —X¹-L²-X²— is selected from the group consisting of

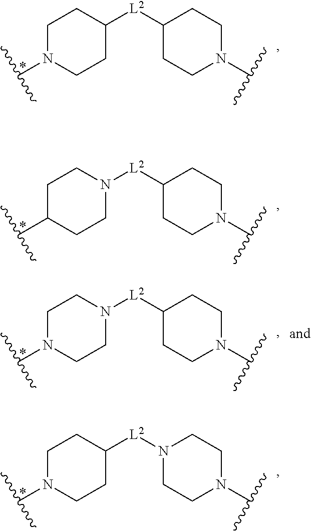

wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen, or a pharmaceutically acceptable salt, hydrate, solvate, prodrug, stereoisomer, or tautomer thereof, wherein * denotes the point of attachment to L¹. In an embodiment, —X¹-L²-X²— is

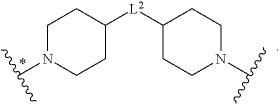

In an embodiment, L² is selected from the group consisting of O, C₁₋₆ alkylene, and C₁₋₆ heteroalkylene. In an embodiment, L² is —CH₂—, O, or C₁₋₃ heteroalkylene. In an embodiment, L² is oxygen. In an embodiment, L₂ is —CH₂—.

In an embodiment, each R^{a} is halogen. In an embodiment, each R^{a} is fluoro.

In an embodiment, —X¹-L²-X²— forms a spiroheterocyclyl having the structure

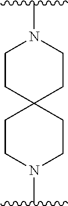 [SMILES: *N1CCC2(CC1)CCN(*)CC2]

substituted with 0-4 occurrences of R^{b}, wherein each R^{b} is independently selected from the group consisting of C₁₋₆ alkyl, C₁₋₆ alkoxyl, and C₁₋₆ hydroxyalkyl.

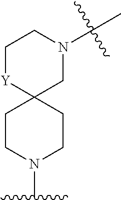

In an embodiment, —X¹-L²-X²— forms a spiroheterocyclyl having the structure substituted with 0-4 occurrences of R^{c}, wherein Y is selected from the group consisting of CH₂, oxygen, and —NR^{c}; and each R^{c} is independently selected from the group consisting of C₁₋₆ alkyl, C₁₋₆ alkoxyl, and C₁₋₆ hydroxyalkyl. In an embodiment, Y is CH₂, CH(C₁₋₃ alkyl), C(C₁₋₃ alkyl)₂, oxygen, NH, or N(C₁₋₃ alkyl).

In an embodiment, L³ is selected from the group consisting of O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O , wherein * denotes the point of attachment of L³ to X². In an embodiment, L³ is selected from the group consisting of O, C(O), C₁₋₆ alkylene, C₁₋₆ heteroalkylene, and *C(O)—C₁₋₆ alkylene-O. In an embodiment, L³ is selected from the group consisting of O, C(O), C₁₋₃ alkylene, C₁₋₃ heteroalkylene, and *C(O)—C₁₋₃ alkylene-O . In an embodiment, L³ is selected from the group consisting of bond, C₁₋₆ alkylene, C₁₋₆ heteroalkylene, *C(O)—C₁₋₆ alkylene, and *C(O)—C₁₋₆ heteroalkylene. In an embodiment, L³ is selected from the group consisting of C₁₋₆ alkylene, C₁₋₆ heteroalkylene, *C(O)—C₁₋₆ alkylene, and *C(O)—C₁₋₆ heteroalkylene.

In an embodiment, the Linker is a compound having the following formula

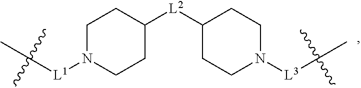

wherein each piperidinyl is substituted with 0-4 occurrences of R^{a}, wherein each R^{a} is independently selected from the group consisting of C₁₋₆ alkyl, C₁₋₆ alkoxyl, and halogen. In an embodiment, L¹ and L³ are each C₁₋₆ alkylene. In an embodiment, L¹ and L³ are each methylene. In an embodiment, L¹ and L³ are each ethylene. In an embodiment, L is methylene and L³ is ethylene. In an embodiment, L² is —CH₂—, O, or C₁₋₃ heteroalkylene. In an embodiment, L² is oxygen. In an embodiment, L₂ is —CH₂—. In an embodiment, each R^{a} is halogen. In an embodiment, each R^{a} is fluoro.

In an embodiment, the Linker is selected from the group consisting of.

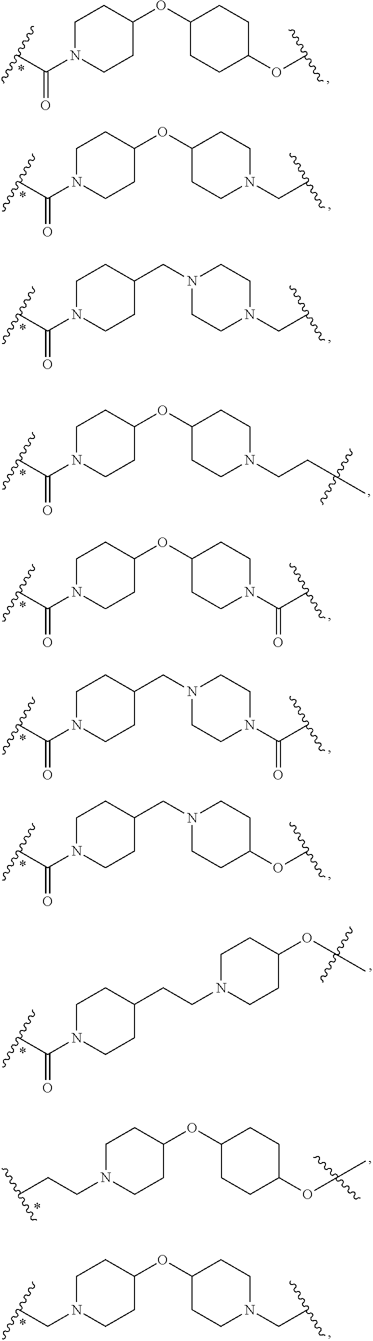

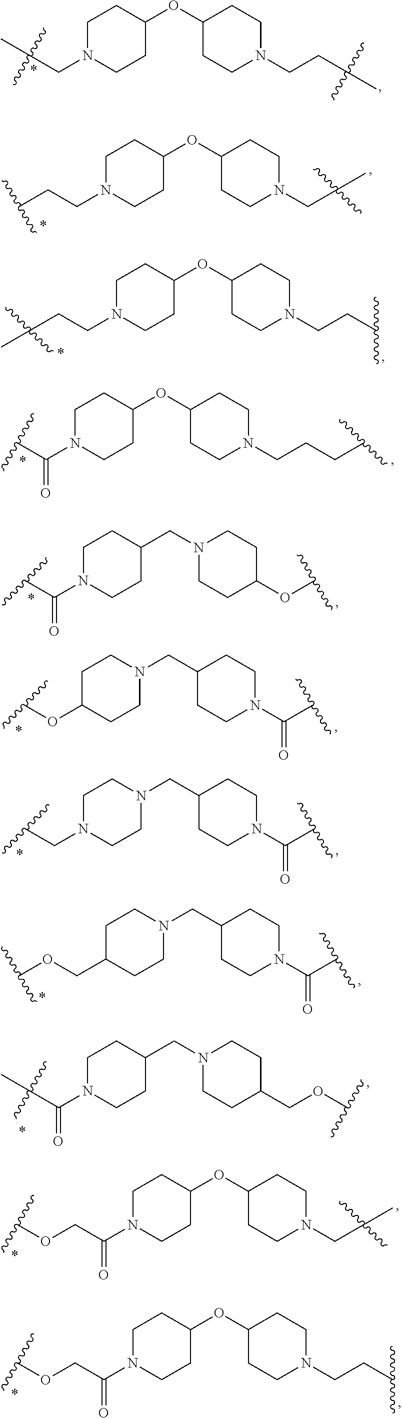

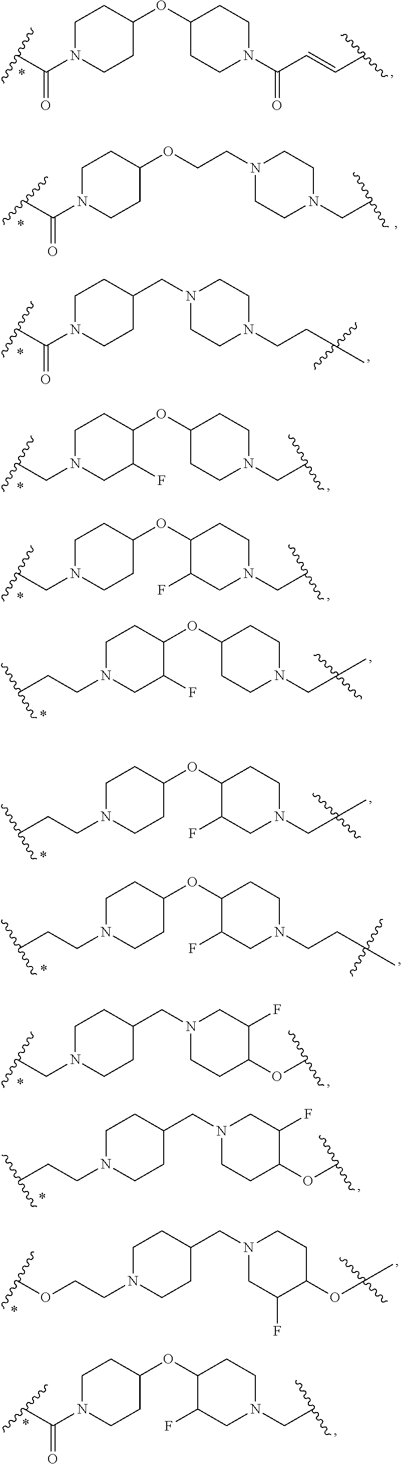

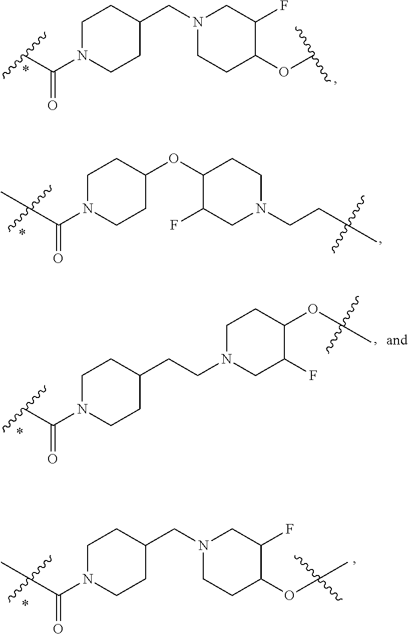 [SMILES: *OC1CCN(CC2CCN(C(*)=O)CC2)CC1F ; *CCN1CCC(OC2CCN(C(*)=O)CC2)C(F)C1 ; *OC1CCN(CCC2CCN(C(*)=O)CC2)CC1F ; *OC1CCN(CC2CCN(C(*)=O)CC2)CC1F]

or a pharmaceutically acceptable salt, hydrate, solvate, prodrug, stereoisomer, or tautomer thereof, wherein * denotes the point of attachment to the Targeting Ligase Binder.

In an embodiment, the Linker is selected from the group consisting of:

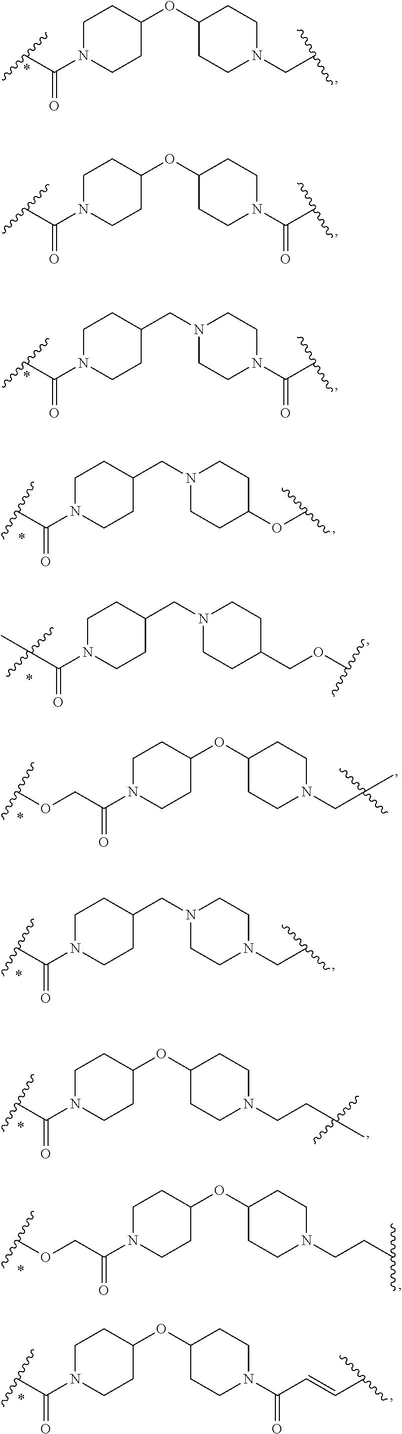

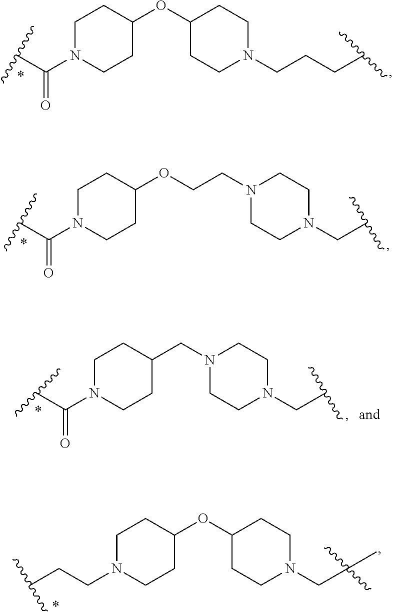 [SMILES: *CCCN1CCC(OC2CCN(C(*)=O)CC2)CC1 ; *CN1CCN(CCOC2CCN(C(*)=O)CC2)CC1 ; *CN1CCN(CC2CCN(C(*)=O)CC2)CC1 ; *CCN1CCC(OC2CCN(C*)CC2)CC1]

or a pharmaceutically acceptable salt, hydrate, solvate, prodrug, stereoisomer, or tautomer thereof, wherein * denotes the point of attachment to the Targeting Ligase Binder.

In an embodiment, the Linker is selected from the group consisting of.

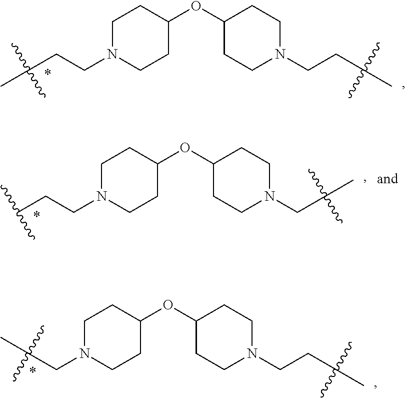 [SMILES: *CCN1CCC(OC2CCN(CC*)CC2)CC1 ; *CCN1CCC(OC2CCN(C*)CC2)CC1 ; *CCN1CCC(OC2CCN(C*)CC2)CC1]

or a pharmaceutically acceptable salt, hydrate, solvate, prodrug, stereoisomer, or tautomer thereof, wherein * denotes the point of attachment to the Targeting Ligase Binder.

In an embodiment, R⁴ is halogen, e.g., chloro or fluoro. In an embodiment, R⁴ is C₁₋₆ alkyl, e.g., methyl. In an embodiment, R⁴ is C₁₋₆ alkoxyl, e.g., methoxyl. In an embodiment, R⁴ is OH. In an embodiment, m is 0. In an embodiment, m is 1. In an embodiment, m is 2. In an embodiment, R⁴ is halogen, e.g., chloro, and m is 1. In an embodiment, R⁴ is C₁₋₆ alkyl, e.g., methyl, and m is 1. In an embodiment, R⁴ is C₁₋₆ alkoxyl, e.g., methoxyl, and m is 1. In an embodiment, R⁴ is OH, and m is 1.

In an embodiment, the Targeting Ligase Binder-Linker has Formula (TLBL-II):

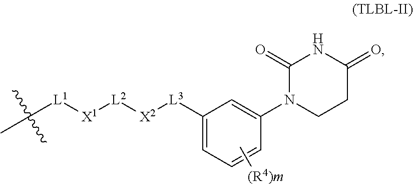

or a pharmaceutically acceptable salt, hydrate, solvate, prodrug, stereoisomer, or tautomer thereof, wherein the point of attachment to the Targeting Ligand is through L¹.

In an embodiment, the Targeting Ligase Binder-Linker has Formula (TLBL-III):

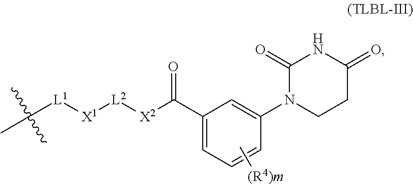

or a pharmaceutically acceptable salt, hydrate, solvate, prodrug, stereoisomer, or tautomer thereof, wherein the point of attachment to the Targeting Ligand is through L¹.

In an embodiment, the Targeting Ligase Binder-Linker has Formula (TLBL-IV):

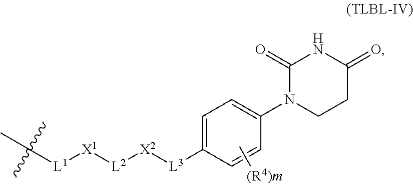

or a pharmaceutically acceptable salt, hydrate, solvate, prodrug, stereoisomer, or tautomer thereof, wherein the point of attachment to the Targeting Ligand is through L¹.

In an embodiment, the Targeting Ligase Binder-Linker has Formula (TLBL-V):

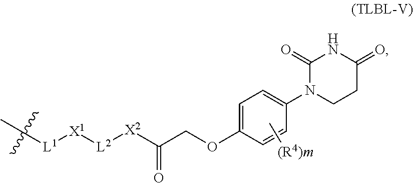

or a pharmaceutically acceptable salt, hydrate, solvate, prodrug, stereoisomer, or tautomer thereof, wherein the point of attachment to the Targeting Ligand is through L¹.

In an embodiment, the Targeting Ligase Binder-Linker has Formula (TLBL-VI):

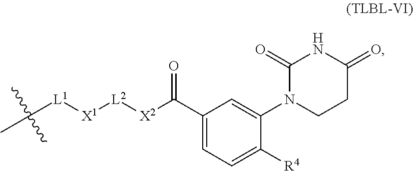

or a pharmaceutically acceptable salt, hydrate, solvate, prodrug, stereoisomer, or tautomer thereof, wherein the point of attachment to the Targeting Ligand is through L¹.

In an embodiment, the Targeting Ligase Binder-Linker or a pharmaceutically acceptable salt, hydrate, solvate, prodrug, stereoisomer, or tautomer thereof, has a formula selected from the group consisting of:

| Structure | |
|---|---|
| Number | Structure |
| TLBL-VII | 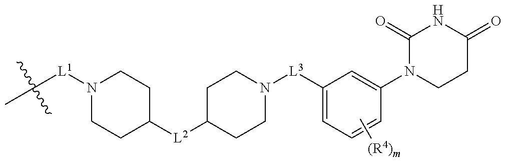 |
| TLBL-VIII | 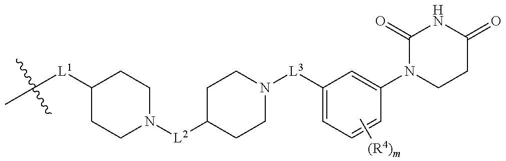 |
| TLBL-IX | 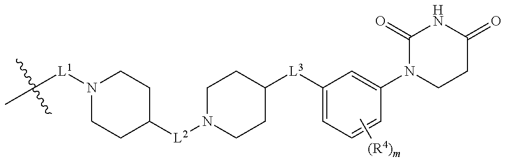 |
| TLBL-X | 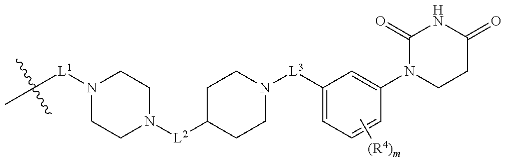 |
| TLBL-XI | 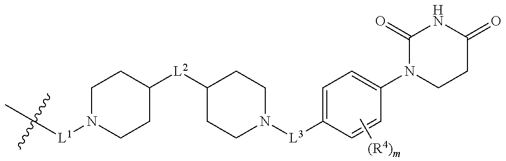 |
| TLBL-XII | 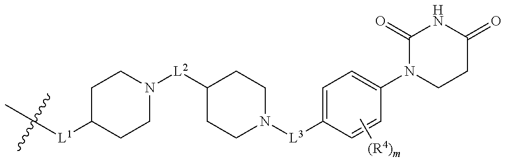 |
| TLBL-XIII | 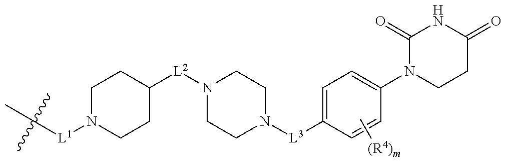 |
| TLBL-XIV | 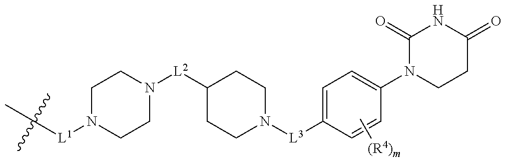 |
| TLBL-XV | 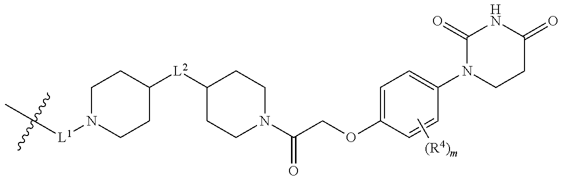 |
| TLBL-XVI | 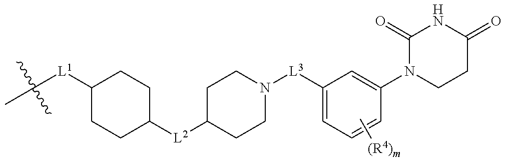 |
| TLBL-XVII | 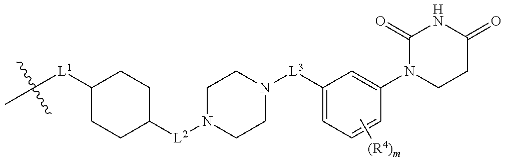 |
| TLBL-XVIII | 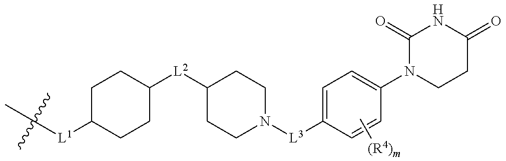 |
| TLBL-XIX | 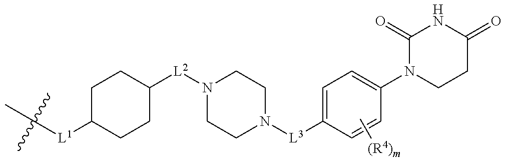 |

wherein R⁴, m, L¹, L², and L³ are each as defined herein.

In an embodiment, the Targeting Ligase Binder-Linker or a pharmaceutically acceptable salt, hydrate, solvate, prodrug, stereoisomer, or tautomer thereof, is selected from the group consisting of:

| Structure | |
|---|---|
| Number | Structure |
| TLBL-1 | 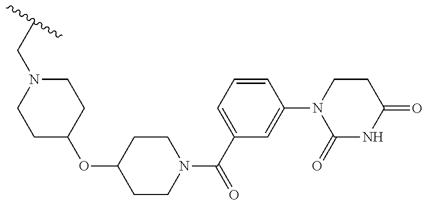 [SMILES: *CN1CCC(OC2CCN(C(=O)c3cccc(N4CCC(=O)NC4=O)c3)CC2)CC1] |
| TLBL-2 | 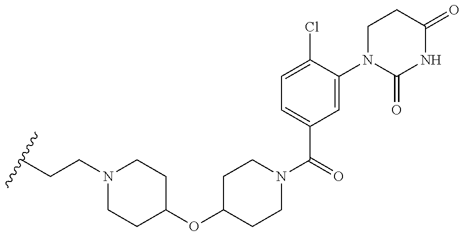 [SMILES: *CCN1CCC(OC2CCN(C(=O)c3ccc(Cl)c(N4CCC(=O)NC4=O)c3)CC2)CC1] |
| TLBL-3 | 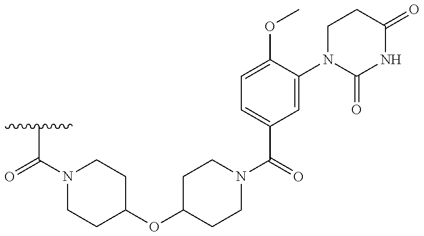 [SMILES: *C(=O)N1CCC(OC2CCN(C(=O)c3ccc(OC)c(N4CCC(=O)NC4=O)c3)CC2)CC1] |
| TLBL-4 | 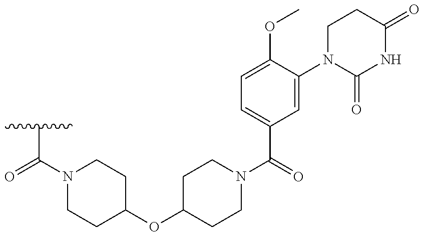 [SMILES: *C(=O)N1CCC(OC2CCN(C(=O)c3ccc(OC)c(N4CCC(=O)NC4=O)c3)CC2)CC1] |
| TLBL-5 | 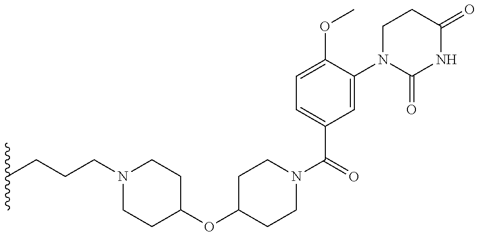 [SMILES: *CCCN1CCC(OC2CCN(C(=O)c3ccc(OC)c(N4CCC(=O)NC4=O)c3)CC2)CC1] |
| TLBL-6 | 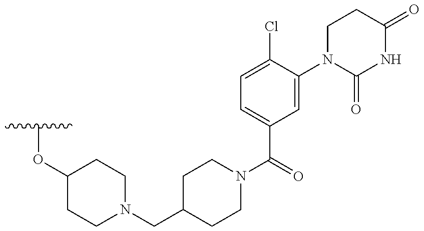 [SMILES: *OC1CCN(CC2CCN(C(=O)c3ccc(Cl)c(N4CCC(=O)NC4=O)c3)CC2)CC1] |
| TLBL-7 | 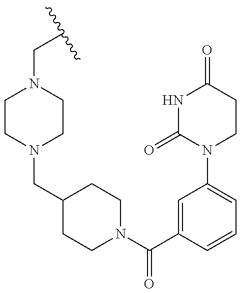 [SMILES: *CN1CCN(CC2CCN(C(=O)c3cccc(N4CCC(=O)NC4=O)c3)CC2)CC1] |
| TLBL-8 | 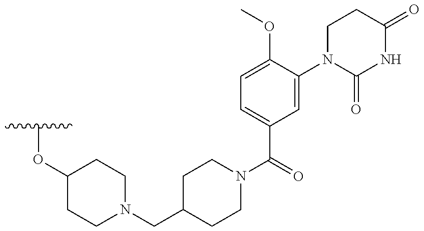 [SMILES: *OC1CCN(CC2CCN(C(=O)c3ccc(OC)c(N4CCC(=O)NC4=O)c3)CC2)CC1] |
| TLBL-9 | 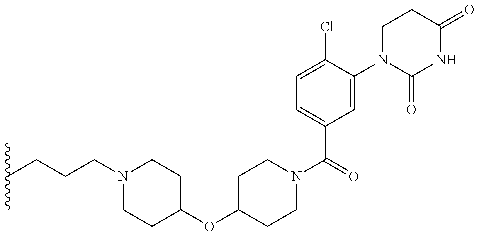 [SMILES: *CCCN1CCC(OC2CCN(C(=O)c3ccc(Cl)c(N4CCC(=O)NC4=O)c3)CC2)CC1] |
| TLBL-10 | 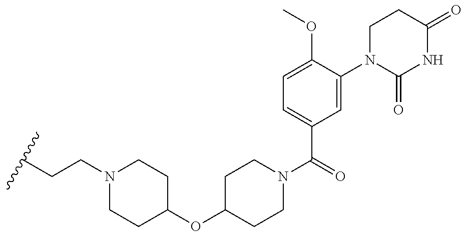 [SMILES: *CCN1CCC(OC2CCN(C(=O)c3ccc(OC)c(N4CCC(=O)NC4=O)c3)CC2)CC1] |
| TLBL-11 | 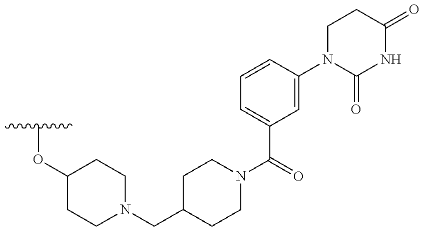 [SMILES: *OC1CCN(CC2CCN(C(=O)c3cccc(N4CCC(=O)NC4=O)c3)CC2)CC1] |
| TLBL-12 | 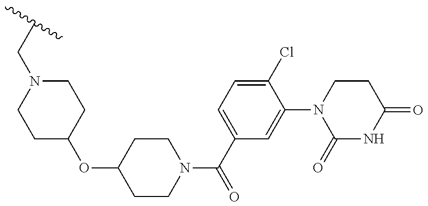 [SMILES: *CN1CCC(OC2CCN(C(=O)c3ccc(Cl)c(N4CCC(=O)NC4=O)c3)CC2)CC1] |
| TLBL-13 | 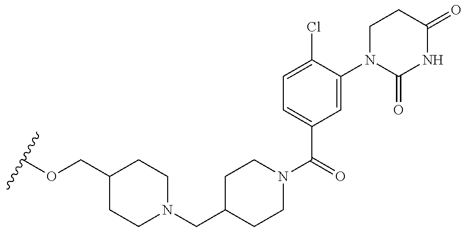 [SMILES: *OCC1CCN(CC2CCN(C(=O)c3ccc(Cl)c(N4CCC(=O)NC4=O)c3)CC2)CC1] |
| TLBL-14 | 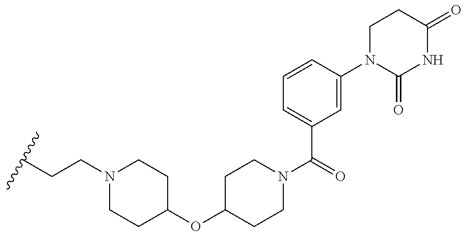 [SMILES: *CCN1CCC(OC2CCN(C(=O)c3cccc(N4CCC(=O)NC4=O)c3)CC2)CC1] |
| TLBL-15 | 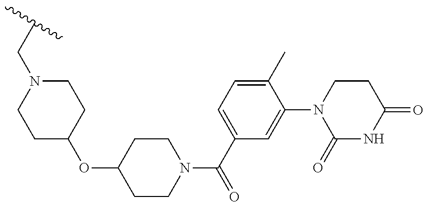 [SMILES: *CN1CCC(OC2CCN(C(=O)c3ccc(C)c(N4CCC(=O)NC4=O)c3)CC2)CC1] |
| TLBL-16 | 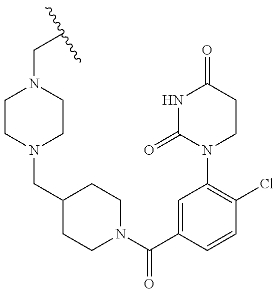 [SMILES: *CN1CCN(CC2CCN(C(=O)c3ccc(Cl)c(N4CCC(=O)NC4=O)c3)CC2)CC1] |
| TLBL-17 | 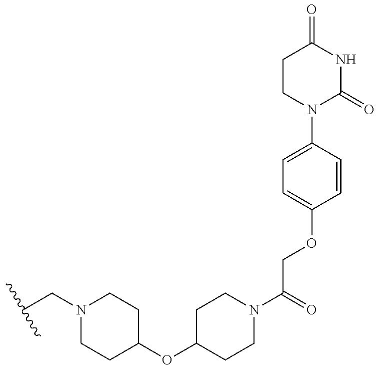 [SMILES: *CN1CCC(OC2CCN(C(=O)COc3ccc(N4CCC(=O)NC4=O)cc3)CC2)CC1] |
| TLBL-18 | 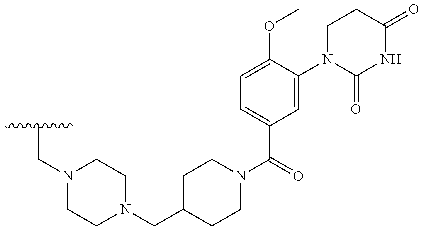 [SMILES: *CN1CCN(CC2CCN(C(=O)c3ccc(OC)c(N4CCC(=O)NC4=O)c3)CC2)CC1] |
| TLBL-19 | 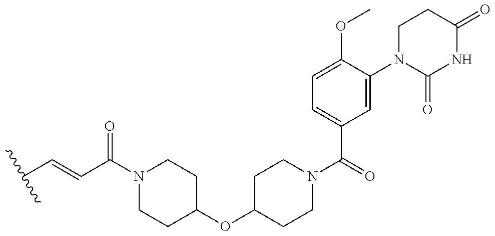 [SMILES: */C=C/C(=O)N1CCC(OC2CCN(C(=O)c3ccc(OC)c(N4CCC(=O)NC4=O)c3)CC2)CC1] |
| TLBL-20 | 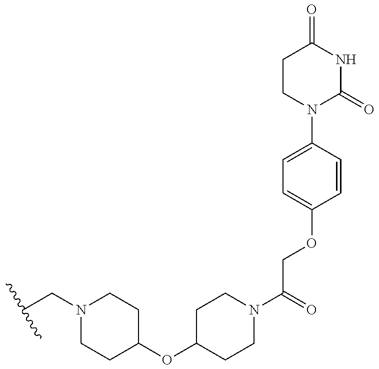 [SMILES: *CN1CCC(OC2CCN(C(=O)COc3ccc(N4CCC(=O)NC4=O)cc3)CC2)CC1] |
| TLBL-21 | 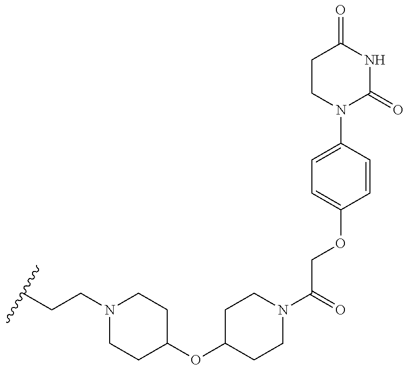 [SMILES: *CCN1CCC(OC2CCN(C(=O)COc3ccc(N4CCC(=O)NC4=O)cc3)CC2)CC1] |
| TLBL-22 | 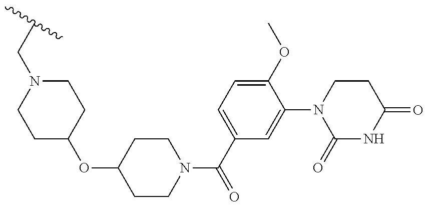 [SMILES: *CN1CCC(OC2CCN(C(=O)c3ccc(OC)c(N4CCC(=O)NC4=O)c3)CC2)CC1] |
| TLBL-23 | 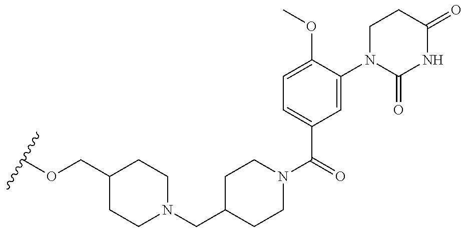 [SMILES: *OCC1CCN(CC2CCN(C(=O)c3ccc(OC)c(N4CCC(=O)NC4=O)c3)CC2)CC1] |
| TLBL-24 | 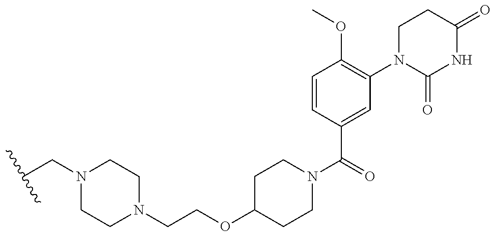 [SMILES: *CN1CCN(CCOC2CCN(C(=O)c3ccc(OC)c(N4CCC(=O)NC4=O)c3)CC2)CC1] |
| TLBL-25 | 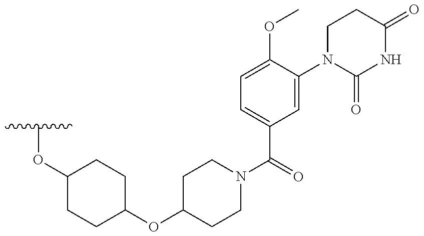 [SMILES: *OC1CCC(OC2CCN(C(=O)c3ccc(OC)c(N4CCC(=O)NC4=O)c3)CC2)CC1] |
| TLBL-26 | 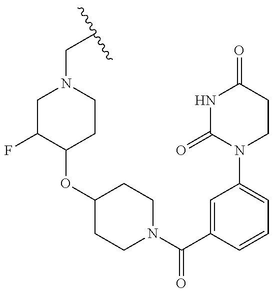 [SMILES: *CN1CCC(OC2CCN(C(=O)c3cccc(N4CCC(=O)NC4=O)c3)CC2)C(F)C1] |
| TLBL-27 | 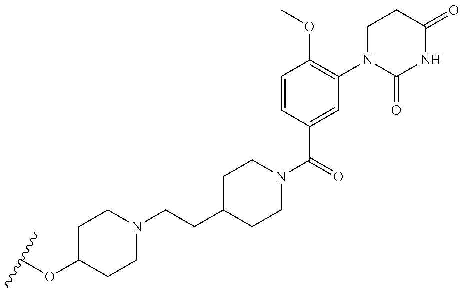 [SMILES: *OC1CCN(CCC2CCN(C(=O)c3ccc(OC)c(N4CCC(=O)NC4=O)c3)CC2)CC1] |
| TLBL-28 | 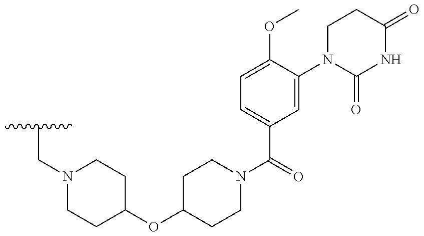 [SMILES: *CN1CCC(OC2CCN(C(=O)c3ccc(OC)c(N4CCC(=O)NC4=O)c3)CC2)CC1] |
| TLBL-29 | 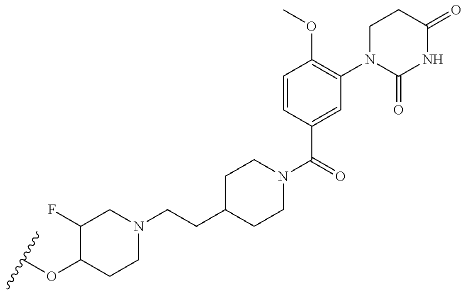 [SMILES: *OC1CCN(CCC2CCN(C(=O)c3ccc(OC)c(N4CCC(=O)NC4=O)c3)CC2)CC1F] |
| TLBL-30 | 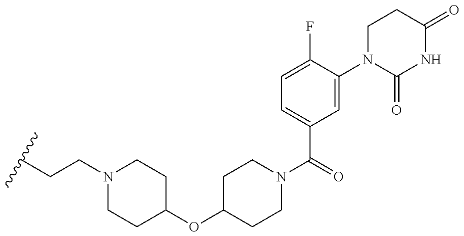 [SMILES: *CCN1CCC(OC2CCN(C(=O)c3ccc(F)c(N4CCC(=O)NC4=O)c3)CC2)CC1] |
| TLBL-31 | 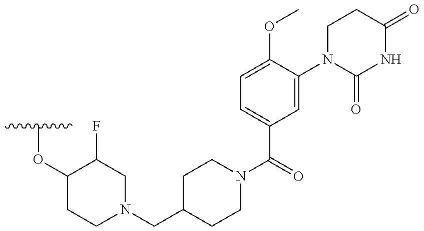 [SMILES: *OC1CCN(CC2CCN(C(=O)c3ccc(OC)c(N4CCC(=O)NC4=O)c3)CC2)CC1F] |
| TLBL-32 | 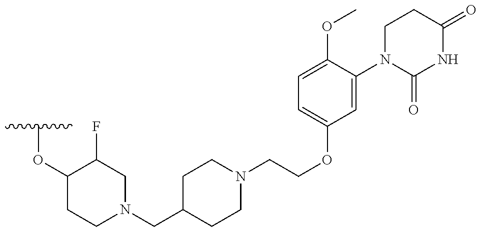 [SMILES: *OC1CCN(CC2CCN(CCOc3ccc(OC)c(N4CCC(=O)NC4=O)c3)CC2)CC1F] |
| TLBL-33 | 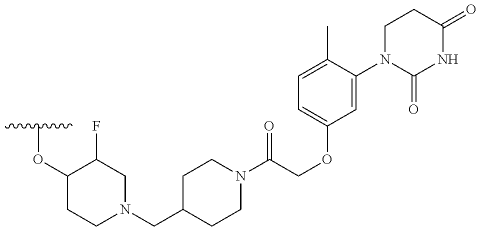 [SMILES: *OC1CCN(CC2CCN(C(=O)COc3ccc(C)c(N4CCC(=O)NC4=O)c3)CC2)CC1F] |
| TLBL-34 | 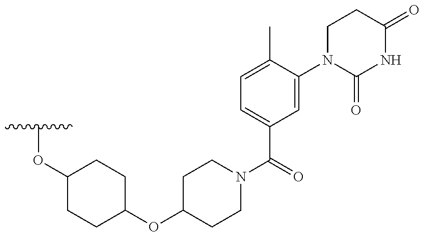 [SMILES: *OC1CCC(OC2CCN(C(=O)c3ccc(C)c(N4CCC(=O)NC4=O)c3)CC2)CC1] |
| TLBL-35 | 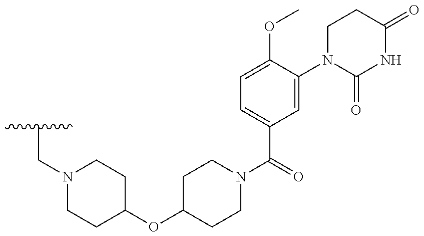 [SMILES: *CN1CCC(OC2CCN(C(=O)c3ccc(OC)c(N4CCC(=O)NC4=O)c3)CC2)CC1] |
| TLBL-36 | 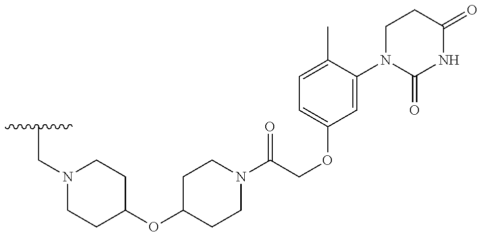 [SMILES: *CN1CCC(OC2CCN(C(=O)COc3ccc(C)c(N4CCC(=O)NC4=O)c3)CC2)CC1] |
| TLBL-37 | 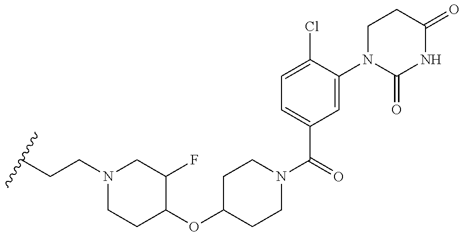 [SMILES: *CCN1CCC(OC2CCN(C(=O)c3ccc(Cl)c(N4CCC(=O)NC4=O)c3)CC2)C(F)C1] |
| TLBL-38 | 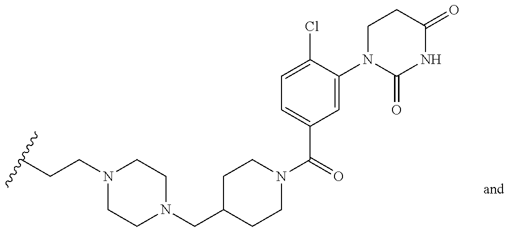 [SMILES: *CCN1CCN(CC2CCN(C(=O)c3ccc(Cl)c(N4CCC(=O)NC4=O)c3)CC2)CC1] |
| TLBL-39 | 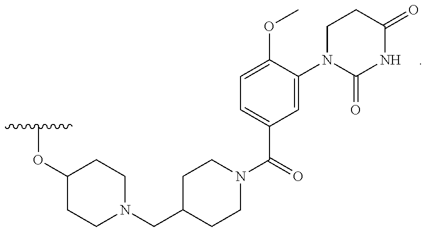 [SMILES: *OC1CCN(CC2CCN(C(=O)c3ccc(OC)c(N4CCC(=O)NC4=O)c3)CC2)CC1] |

In an embodiment, R^{d1} and R^{d2} are both methyl. In an embodiment, R^{d1} and R² are both H. In an embodiment, R^{d4} is H or C₁₋₆alkyl, e.g., methyl. In an embodiment, R^{d5} is H or C₁₋₆ alkyl, e.g., methyl.

In an embodiment, R^{d4} is H or C₁₋₆alkyl, e.g., methyl. In an embodiment, R^{d5} is H or C₁₋₆ alkyl, e.g., methyl.

In an embodiment, R^{d1}, R^{d2}, R^{d4}, and R^{d5} are each H.

In an embodiment, the Targeting Ligase Binder-Linker has Formula (TLBL-II′):

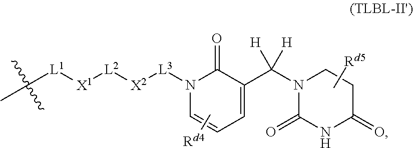

or a pharmaceutically acceptable salt, hydrate, solvate, prodrug, stereoisomer, or tautomer thereof, wherein the point of attachment to the Targeting Ligand is through L¹.

In an embodiment, the Targeting Ligase Binder-Linker has Formula (TLBL-III′):

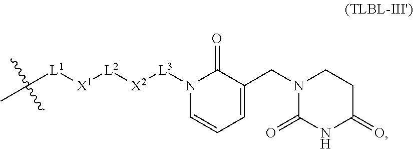

or a pharmaceutically acceptable salt, hydrate, solvate, prodrug, stereoisomer, or tautomer thereof, wherein the point of attachment to the Targeting Ligand is through L¹.

In an embodiment, the Targeting Ligase Binder-Linker or a pharmaceutically acceptable salt, hydrate, solvate, prodrug, stereoisomer, or tautomer thereof, has a formula selected from the group consisting of:

| Structure | |
|---|---|
| Number | Structure |
| TLBL-IV′ | 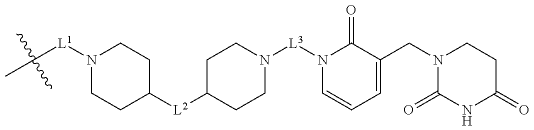 |
| TLBL-V′ | 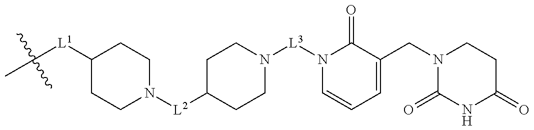 |
| TLBL-VI′ | 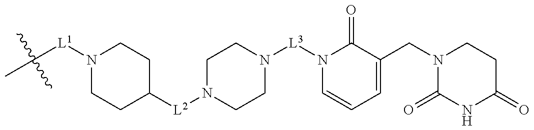 |
| TLBL-VII′ | 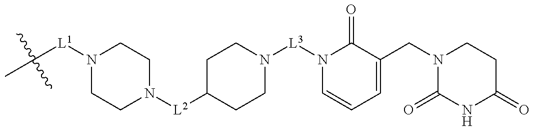 |
| TLBL-VIII′ | 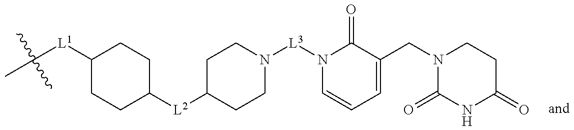 |
| TLBL-IX′ | 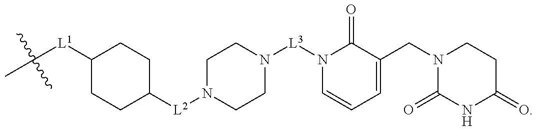 |

wherein L¹, L², and L³ are each as defined herein.

In an embodiment, the Targeting Ligase Binder-Linker or a pharmaceutically acceptable salt, hydrate, solvate, prodrug, stereoisomer, or tautomer thereof, is selected from the group consisting of:

| Structure | |
|---|---|
| Number | Structure |
| TLBL-X′ | 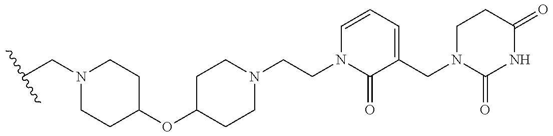 [SMILES: *CN1CCC(OC2CCN(CCn3cccc(CN4CCC(=O)NC4=O)c3=O)CC2)CC1] |
| TLBL-XI′ | 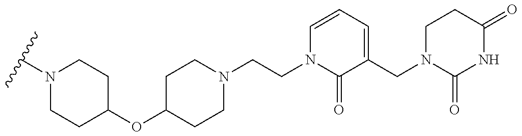 [SMILES: *N1CCC(OC2CCN(CCn3cccc(CN4CCC(=O)NC4=O)c3=O)CC2)CC1] |
| TLBL-XII′ | 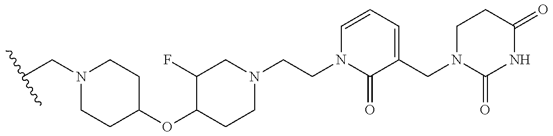 [SMILES: *CN1CCC(OC2CCN(CCn3cccc(CN4CCC(=O)NC4=O)c3=O)CC2F)CC1] |
| TLBL-XIII′ | 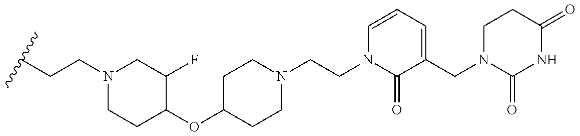 [SMILES: *CCN1CCC(OC2CCN(CCn3cccc(CN4CCC(=O)NC4=O)c3=O)CC2)C(F)C1] |
| TLBL-XIV′ | 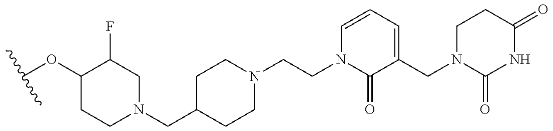 [SMILES: *OC1CCN(CC2CCN(CCn3cccc(CN4CCC(=O)NC4=O)c3=O)CC2)CC1F] |
| TLBL-XV′ | 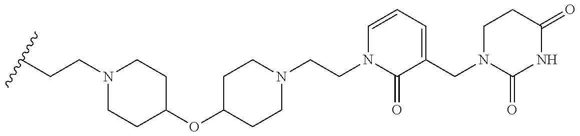 [SMILES: *CCN1CCC(OC2CCN(CCn3cccc(CN4CCC(=O)NC4=O)c3=O)CC2)CC1] |
| TLBL-XVI′ | 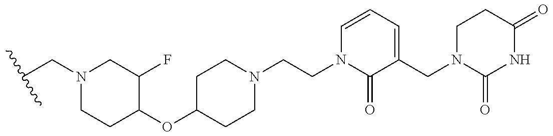 [SMILES: *CN1CCC(OC2CCN(CCn3cccc(CN4CCC(=O)NC4=O)c3=O)CC2)C(F)C1] |
| TLBL-XVII′ | 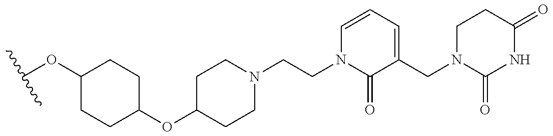 [SMILES: *OC1CCC(OC2CCN(CCn3cccc(CN4CCC(=O)NC4=O)c3=O)CC2)CC1] |

### Structure of Targeting Ligand-Linkers

In another aspect, the Targeting Ligand-Linker is a compound of Formula (TLL-I):

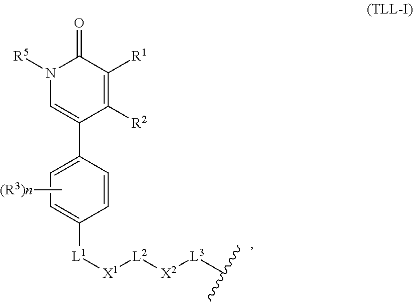

or a pharmaceutically acceptable salt, hydrate, solvate, prodrug, stereoisomer, or tautomer thereof, wherein:

R¹ and R² are independently selected from the group consisting of hydrogen and C₁₋₆ alkyl; or R¹ and R² together with the atoms to which they are attached form an aryl or heteroaryl;

R³ are each independently selected from the group consisting of C₁₋₆ alkyl, C₁₋₆ alkoxyl, and halogen;

R⁵ is selected from the group consisting of hydrogen and C₁₋₆ alkyl;

L¹ is selected from the group consisting of a bond, O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the phenyl ring in Formula (TLL-I);

X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;

L² is selected from the group consisting of a bond, O, NR′, C₁₋₆ alkylene, and C₁₋₆ heteroalkylene; or

X¹-L²-X² form a spiroheterocyclyl;

L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O , wherein * denotes the point of attachment of L³ to X² in Formula (TLL-I); wherein no more than 2 of L¹, X¹, X², L², and L³ can simultaneously be a bond;

R′ is selected from the group consisting of hydrogen and C₁₋₆ alkyl;

n is 0, 1, or 2; and wherein the point of attachment to the Targeting Ligase Binder is through L³.

In another aspect, the Targeting Ligand-Linker is a compound of Formula (TLL-I′):

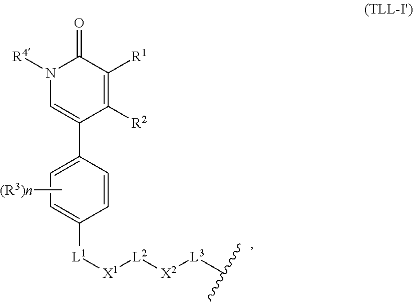

or a pharmaceutically acceptable salt, hydrate, solvate, prodrug, stereoisomer, or tautomer thereof, wherein:

R¹ and R² are independently selected from the group consisting of hydrogen and C₁₋₆ alkyl; or R¹ and R² together with the atoms to which they are attached form an aryl or heteroaryl;

R³ are each independently selected from the group consisting of C₁₋₆ alkyl, C₁₋₆ alkoxyl, and halogen;

R′ is selected from the group consisting of hydrogen or C₁₋₆ alkyl;

L¹ is selected from the group consisting of a bond, O, NR′, C(O), C₁₋₆ alkylene, C₁₋₆ heteroalkylene, *C(O)—C₁₋₆ alkylene, and *C(O)—C₁₋₆ heteroalkylene, wherein * denotes the point of attachment of L¹ to the phenyl ring in Formula (TLL-I′);

X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;

L² is selected from the group consisting of a bond, O, NR′, C₁₋₆ alkylene, and C₁₋₆ heteroalkylene; or

X¹-L²-X² form a spiroheterocyclyl;

L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O , wherein * denotes the point of attachment of L³ to X² in Formula (TLL-I′); wherein no more than 2 of L¹, X¹, X², L², and L³ can simultaneously be a bond;

R′ is selected from the group consisting of hydrogen and C₁₋₆ alkyl; and

n is 0, 1, or 2; and wherein the point of attachment to the Targeting Ligase Binder is through L³.

In an embodiment of Targeting Ligand-Linker, R¹ and R² together with the atoms to which they are attached form an aryl or heteroaryl. In an embodiment, R¹ and R² together with the atoms to which they are attached form a phenyl. In an embodiment, R¹ and R² together with the atoms to which they are attached form a heteroaryl. In an embodiment, R¹ and R² together with the atoms to which they are attached form a 5- or 6-membered heteroaryl. In an embodiment, R¹ and R² together with the atoms to which they are attached form a 6-membered heteroaryl. In an embodiment, R¹ and R² together with the atoms to which they are attached form a 6-membered nitrogen-containing heteroaryl. In an embodiment, R¹ and R² together with the atoms to which they are attached form a pyridyl.

In an embodiment, R³ is independently selected from the group consisting of methoxyl, chloro, and fluoro. In an embodiment, R³ is methoxyl. In an embodiment, R³ is chloro or fluoro.

In an embodiment, n is 1 or 2.

In an embodiment, R³ is methoxyl and n is 1 or 2. In an embodiment, n is 1. In an embodiment, n is 2. In an embodiment, n is 0.

In an embodiment, one of X¹ and X² is not a bond. In an embodiment, one of X¹ and X² is a bond and the other is a carbocyclyl or heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen. In an embodiment, one of X¹ and X² is a bond and the other is a heterocyclyl, wherein the heterocyclyl are substituted with 0-4 occurrences of R^{a}, wherein each R^{a} is independently selected from the group consisting of C₁₋₆ alkyl, C₁₋₆ alkoxyl, and halogen.

In an embodiment, X¹ and X² are each independently selected from the group consisting of cyclohexyl, piperidinyl, and piperazinyl, wherein each cyclohexyl, piperidinyl, and piperazinyl is substituted with 0-4 occurrences of R^{a}, wherein each R^{a} is independently selected from the group consisting of C₁₋₆ alkyl, C₁₋₆ alkoxyl, and halogen.

In an embodiment, —X¹-L²-X²— is selected from the group consisting of

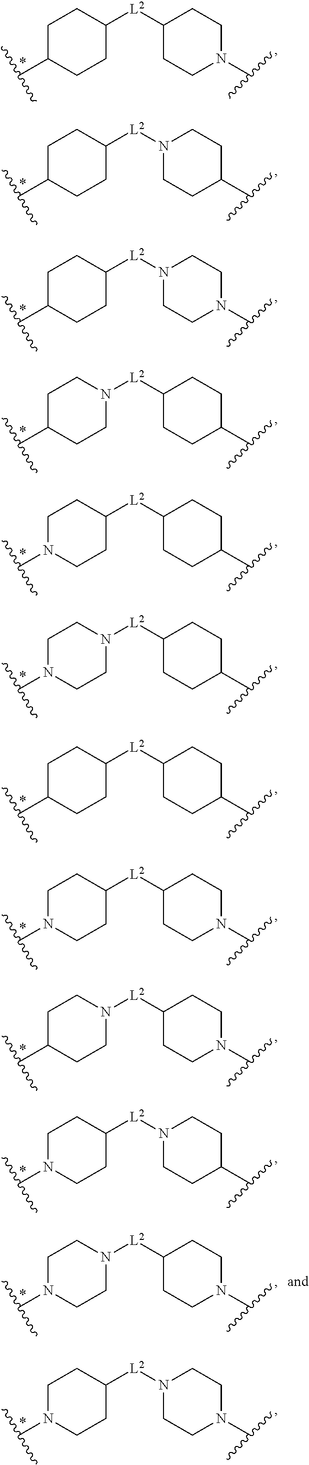

wherein each cyclohexyl, piperidinyl, and piperazinyl is substituted with 0-4 occurrences of R^{a}, wherein each R^{a} is independently selected from the group consisting of C₁₋₆ alkyl, C₁₋₆ alkoxyl, and halogen, or a pharmaceutically acceptable salt, hydrate, solvate, prodrug, stereoisomer, or tautomer thereof, wherein * denotes the point of attachment to L¹.

In an embodiment, X¹ and X² are each independently selected from the group consisting of piperidinyl and piperazinyl, wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen.

In an embodiment, X¹ and X² are both piperidinyl, wherein each piperidinyl is substituted with 0-4 occurrences of R^{a}, wherein each R^{a} is independently selected from the group consisting of C₁₋₆ alkyl, C₁₋₆ alkoxyl, and halogen.

In an embodiment, —X¹-L²-X²— is selected from the group consisting of

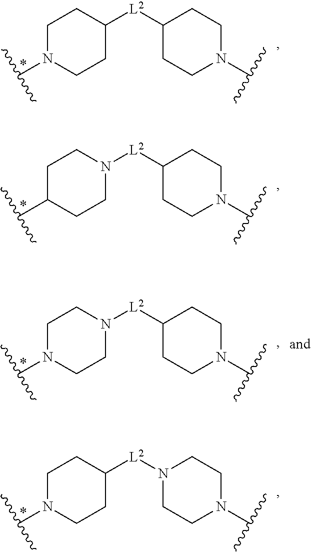

wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen, wherein * denotes the point of attachment to L¹. In an embodiment, X¹-L²-X² is

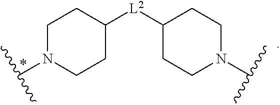

In an embodiment, L² is selected from the group consisting of O, C₁₋₆ alkylene, and C₁₋₆ heteroalkylene. In an embodiment, L² is —CH₂—, O, or C₁₋₃ heteroalkylene. In an embodiment, L² is oxygen. In an embodiment, L₂ is —CH₂—.

In an embodiment, each R^{a} is halogen. In an embodiment, each R^{a} is fluoro.

In an embodiment, L¹ is —O— or C₁₋₆ alkylene.

In an embodiment, R⁴ is halogen, e.g., chloro or fluoro. In an embodiment, R⁴ is C₁₋₆ alkyl, e.g., methyl. In an embodiment, R⁴ is C₁₋₆ alkoxyl, e.g., methoxyl. In an embodiment, R⁴ is OH. In an embodiment, m is 0. In an embodiment, m is 1. In an embodiment, m is 2. In an embodiment, R⁴ is halogen, e.g., chloro, and m is 1. In an embodiment, R⁴ is C₁₋₆ alkyl, e.g., methyl, and m is 1. In an embodiment, R⁴ is C₁₋₆ alkoxyl, e.g., methoxyl, and m is 1. In an embodiment, R⁴ is OH, and m is 1.

In an embodiment, R⁵ is methyl. In an embodiment, R⁵ is n-butyl.

In an embodiment, R^{4′} is methyl. In an embodiment, R^{4′} is n-butyl.

In an embodiment, the Targeting Ligand-Linker or a pharmaceutically acceptable salt, hydrate, solvate, prodrug, stereoisomer, or tautomer thereof, has a formula selected from the group consisting of:

| Struc- | |
|---|---|
| ture | |
| Num- | |
| ber | Structure |
| TLL- II | 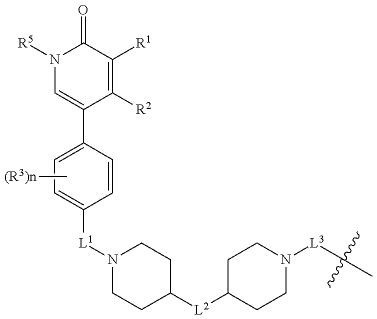 |
| TLL- III | 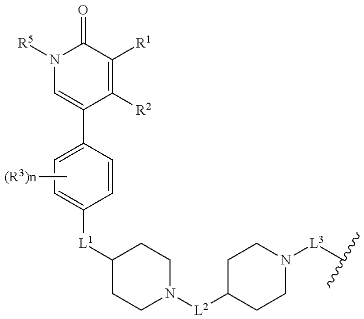 |
| TLL- IV | 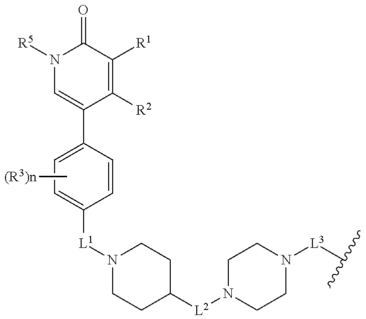 |
| TLL- V | 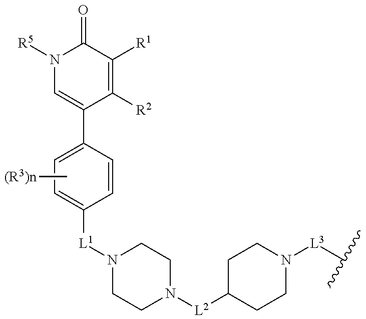 |
| TLL- VI | 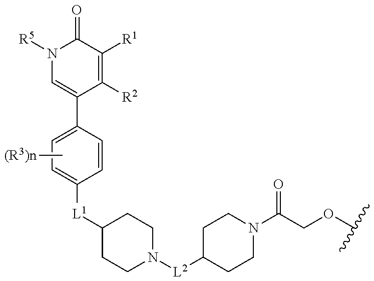 |
| TLL- VII | 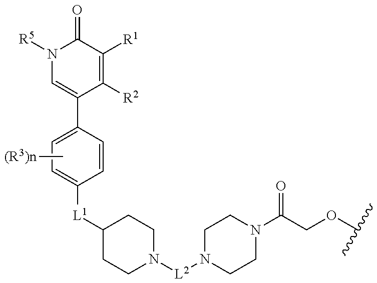 |
| TLL- VIII | 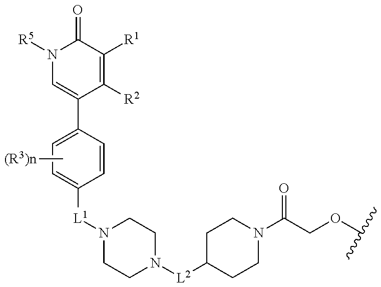 |
| TLL- IX | 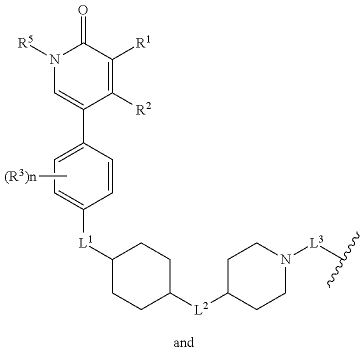 |
| TLL- X | 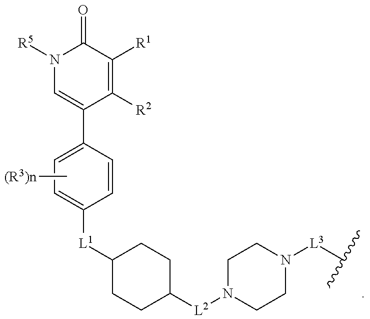 |

wherein R¹, R², R³, R⁵, L¹, L², L³, and n are each as defined herein.

In an embodiment, the Targeting Ligand-Linker or a pharmaceutically acceptable salt, hydrate, solvate, prodrug, stereoisomer, or tautomer thereof, has a formula from the group consisting of:

| Structure | |
|---|---|
| Number | Structure |
| TLL-II′ | 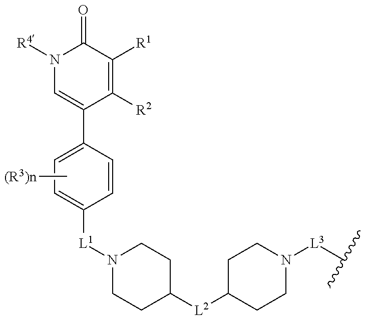 |
| TLL-III′ | 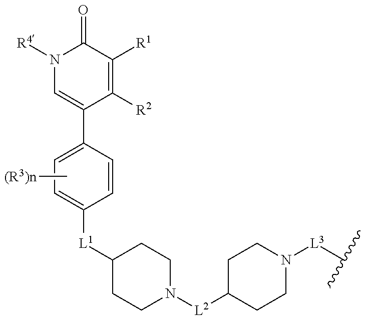 |
| TLL-IV′ | 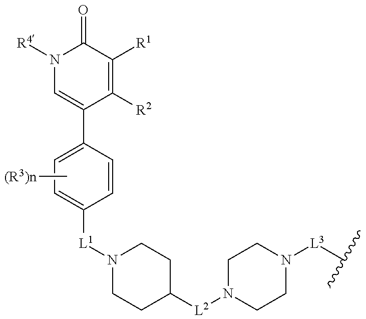 |
| TLL-V′ | 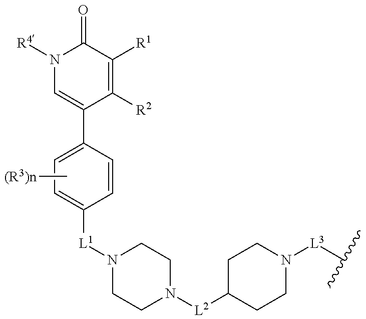 |
| TLL-VI′ | 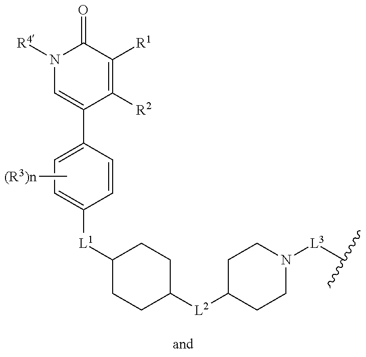 |
| TLL-VIII′ | 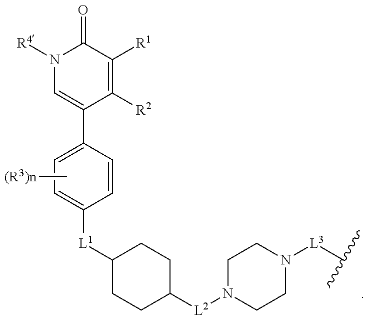 |

wherein R¹, R², R³, n, R^{4′}, L¹, L², L³ and n are each as defined herein.

### Compounds

In one aspect, the compound is a compound of Formula (BF-I):

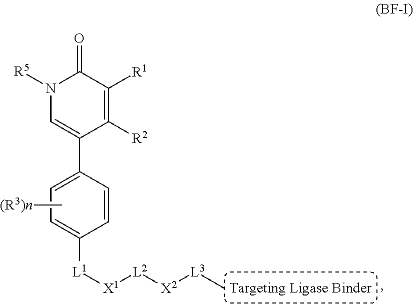

or a pharmaceutically acceptable salt, hydrate, solvate, prodrug, stereoisomer, or tautomer thereof, wherein:

R¹ and R² are independently selected from the group consisting of hydrogen and C₁₋₆ alkyl; or R¹ and R² together with the atoms to which they are attached form an aryl or heteroaryl;

R³ is selected from the group consisting of C₁₋₆ alkyl, C₁₋₆ alkoxyl, and halogen;

L¹ is selected from the group consisting of a bond, O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the phenyl ring in Formula (BF-I);

X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;

L² is selected from the group consisting of a bond, O, NR′, C₁₋₆ alkylene, and C₁₋₆ heteroalkylene; or

X¹-L²-X² form a spiroheterocyclyl;

L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O , wherein * denotes the point of attachment of L³ to X² in Formula (BF-I); wherein no more than 2 of L¹, X¹, X², L², and L³ can simultaneously be a bond;

R⁵ is selected from the group consisting of hydrogen and C₁₋₆ alkyl;

R′ is selected from the group consisting of hydrogen and C₁₋₆ alkyl; and

n is 0, 1, or 2; and

the Targeting Ligase Binder is a group that is capable of binding to a Ubiquitin ligase, e.g., an E3 Ubiquitin ligase, such as Cereblon.

In one aspect, the compound is a compound of Formula (BF-I′):

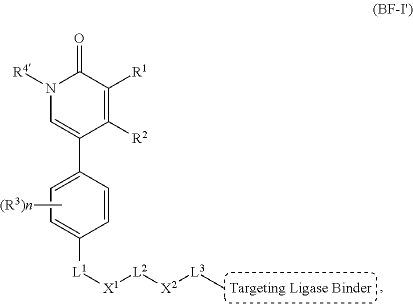

or a pharmaceutically acceptable salt, hydrate, solvate, prodrug, stereoisomer, or tautomer thereof, wherein:

R¹ and R² are independently selected from the group consisting of hydrogen and C₁₋₆ alkyl; or R¹ and R² together with the atoms to which they are attached form an aryl or heteroaryl;

R³ are each independently selected from the group consisting of C₁₋₆ alkyl, C₁₋₆ alkoxyl, and halogen;

L¹ is selected from the group consisting of a bond, O, NR′, C(O), C₁₋₆ alkylene, C₁₋₆ heteroalkylene, *C(O)—C₁₋₆ alkylene, and *C(O)—C₁₋₆ heteroalkylene, wherein * denotes the point of attachment of L¹ to the phenyl ring in Formula (BF-I′);

X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;

L² is selected from the group consisting of a bond, O, NR′, C₁₋₆ alkylene, and C₁₋₆ heteroalkylene; or

X¹-L²-X² form a spiroheterocyclyl;

L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O , wherein * denotes the point of attachment of L³ to X² in Formula (BF-I′); wherein no more than 2 of L¹, X¹, X², L², and L³ can simultaneously be a bond;

R^{4′} is selected from the group consisting of hydrogen or C₁₋₆ alkyl;

R′ is selected from the group consisting of hydrogen and C₁₋₆ alkyl;

n is 0, 1, or 2; and

the Targeting Ligase Binder is a group that is capable of binding to a ubiquitin ligase, e.g., an E3 ubiquitin ligase, such as Cereblon.

In an embodiment of the compounds disclosed herein, R¹ and R² together with the atoms to which they are attached form an aryl or heteroaryl. In an embodiment, R¹ and R² together with the atoms to which they are attached form a phenyl. In an embodiment, R¹ and R² together with the atoms to which they are attached form a heteroaryl. In an embodiment, R¹ and R² together with the atoms to which they are attached form a 5- or 6-membered heteroaryl. In an embodiment, R¹ and R² together with the atoms to which they are attached form a 6-membered heteroaryl. In an embodiment, R¹ and R² together with the atoms to which they are attached form a 6-membered nitrogen-containing heteroaryl. In an embodiment, R¹ and R² together with the atoms to which they are attached form a pyridyl.

In an embodiment, R³ is selected from the group consisting of methoxyl, chloro, and fluoro. In an embodiment, R³ is methoxyl. In an embodiment, R³ is chloro or fluoro.

In an embodiment, n is 1 or 2.

In an embodiment, R³ is methoxyl and n is 1 or 2. In an embodiment, n is 1. In an embodiment, n is 2. In an embodiment, n is 0.

In an embodiment, one of X¹ and X² is not a bond. In an embodiment, one of X¹ and X² is a bond and the other is a carbocyclyl or heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen. In an embodiment, one of X¹ and X² is a bond and the other is a heterocyclyl, wherein the heterocyclyl are substituted with 0-4 occurrences of R^{a}, wherein each R^{a} is independently selected from the group consisting of C₁₋₆ alkyl, C₁₋₆ alkoxyl, and halogen.

In an embodiment, X¹ and X² are each independently selected from the group consisting of cyclohexyl, piperidinyl, and piperazinyl, wherein each cyclohexyl, piperidinyl, and piperazinyl is substituted with 0-4 occurrences of R^{a}, wherein each R^{a} is independently selected from the group consisting of C₁₋₆ alkyl, C₁₋₆ alkoxyl, and halogen.

In an embodiment —X¹-L²-X²— is selected from the group consisting of

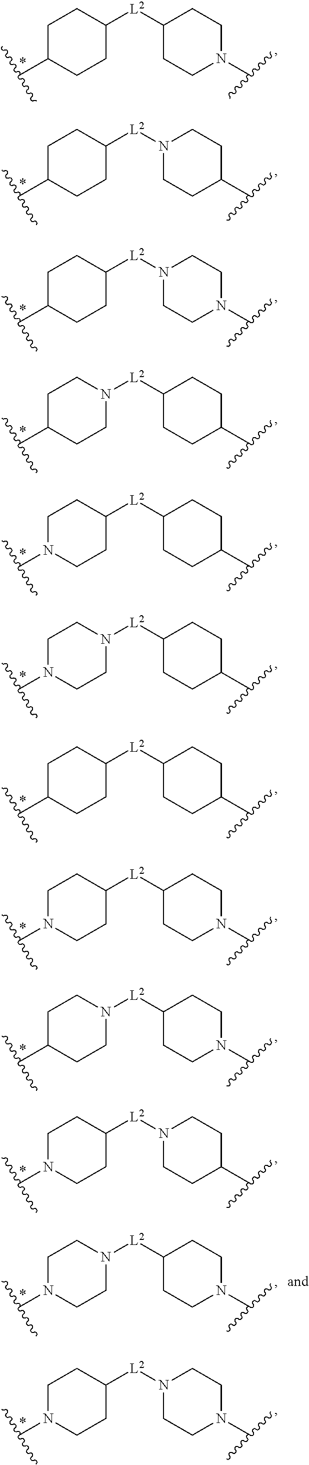

wherein each cyclohexyl, piperidinyl, and piperazinyl is substituted with 0-4 occurrences of R^{a}, wherein each R^{a} is independently selected from the group consisting of C₁₋₆ alkyl, C₁₋₆ alkoxyl, and halogen, or a pharmaceutically acceptable salt, hydrate, solvate, prodrug, stereoisomer, or tautomer thereof, wherein * denotes the point of attachment to L¹.

In an embodiment, X¹ and X² are each independently selected from the group consisting of piperidinyl and piperazinyl, wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen.

In an embodiment, X¹ and X² are both piperidinyl, wherein each piperidinyl is substituted with 0-4 occurrences of R^{a}, wherein each R^{a} is independently selected from the group consisting of C₁₋₆ alkyl, C₁₋₆ alkoxyl, and halogen.

In an embodiment, X¹-L²-X² is selected from the group consisting of:

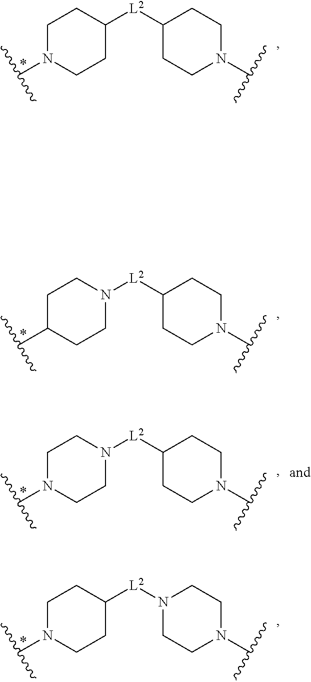

wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen, and wherein * denotes the point of attachment to L¹. In an embodiment, X¹-L²-X² is

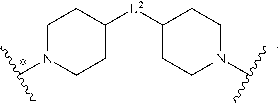

In an embodiment, L² is —CH₂—, O, or C₁₋₃ heteroalkylene. In an embodiment, L² is oxygen. In an embodiment, L² is —CH₂—.

In an embodiment, L¹ is —O— or C₁₋₆ alkylene.

In an embodiment, R⁵ is methyl. In an embodiment, R⁵ is n-butyl.

In an embodiment, R^{4′} is methyl. In an embodiment, R^{4′} is n-butyl.

In another aspect, the compound is a compound of Formula (BF-II):

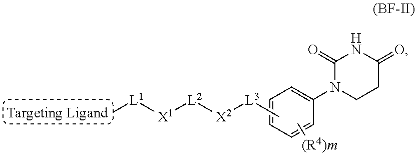

or a pharmaceutically acceptable salt, hydrate, solvate, prodrug, stereoisomer, or tautomer thereof, wherein:

L¹ is selected from the group consisting of a bond, O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the Targeting Ligand in Formula (BF-II);

X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;

L² is selected from the group consisting of a bond, O, NR′, C₁₋₆ alkylene, and C₁₋₆ heteroalkylene; or

X¹-L²-X² form a spiroheterocyclyl;

L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O , wherein * denotes the point of attachment of L³ to X² in Formula (BF-II); wherein no more than 2 of L¹, X¹, X², L², and L³ can simultaneously be a bond; R⁴ is selected from the group consisting of OH, C₁₋₆ alkyl, C₁₋₆ alkoxyl, and halogen;

R′ is selected from the group consisting of hydrogen and C₁₋₆ alkyl; and

m is 0, 1, or 2; and

the Targeting Ligand is a group that is capable of binding to a bromodomain-containing protein, e.g., BRD9.

In another aspect, the compound is a compound of Formula (BF-II′):

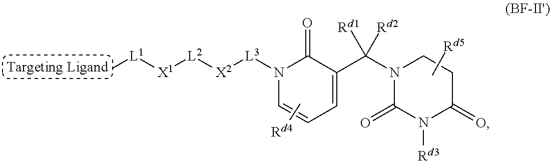

or a pharmaceutically acceptable salt, hydrate, solvate, prodrug, stereoisomer, or tautomer thereof, wherein:

L¹ is selected from the group consisting of a bond, O, NR′, C(O), C₁₋₆ alkylene, C₁₋₆ heteroalkylene, *C(O)—C₁₋₆ alkylene, and *C(O)—C₁₋₆ heteroalkylene, wherein * denotes the point of attachment of L¹ to the Targeting Ligand in Formula (BF-II′);

X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;

L² is selected from the group consisting of a bond, O, NR′, C₁₋₆ alkylene, and C₁₋₆ heteroalkylene; or

X¹-L²-X² form a spiroheterocyclyl;

L³ is selected from the group consisting of a bond, C₁₋₆ alkylene, C₁₋₆ heteroalkylene, *C(O)—C₁₋₆ alkylene, and *C(O)—C₁₋₆ heteroalkylene, wherein * denotes the point of attachment of L³ to X² in Formula (BF-II′); wherein no more than 2 of L¹, X¹, X², L², and L³ can simultaneously be a bond;

R′ is selected from the group consisting of hydrogen and C₁₋₆ alkyl;

R^{d1} and R^{d2} are each independently selected from the group consisting of H, C₁₋₆ alkyl, C₁₋₆ alkoxyl, C₁₋₆ haloalkyl, and C₁₋₆ heteroalkyl;

R^{d3} is H;

R^{d4} is selected from the group consisting of H, C₁₋₆ alkyl, halo, C₁₋₆ haloalkyl, and C₁₋₆ heteroalkyl;

R^{d5} is selected from the group consisting of H, C₁₋₆ alkyl, halo, C₁₋₆ haloalkyl, and C₁₋₆ heteroalkyl; and

the Targeting Ligand is a group that is capable of binding to a bromodomain-containing protein, e.g., BRD9.

In an embodiment of the compounds disclosed herein, one of X¹ and X² is not a bond. In an embodiment, one of X¹ and X² is a bond and the other is a carbocyclyl or heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen. In an embodiment, one of X¹ and X² is a bond and the other is a heterocyclyl, wherein the heterocyclyl is substituted with 0-4 occurrences of R^{a}, wherein each R^{a} is independently selected from the group consisting of C₁₋₆ alkyl, C₁₋₆ alkoxyl, and halogen.

In an embodiment, X¹ and X² are each independently selected from the group consisting of cyclohexyl, piperidinyl, and piperazinyl, wherein each cyclohexyl, piperidinyl, and piperazinyl is substituted with 0-4 occurrences of R^{a}, wherein each R^{a} is independently selected from the group consisting of C₁₋₆ alkyl, C₁₋₆ alkoxyl, and halogen.

In an embodiment, —X¹-L²-X²— is selected from the group consisting of

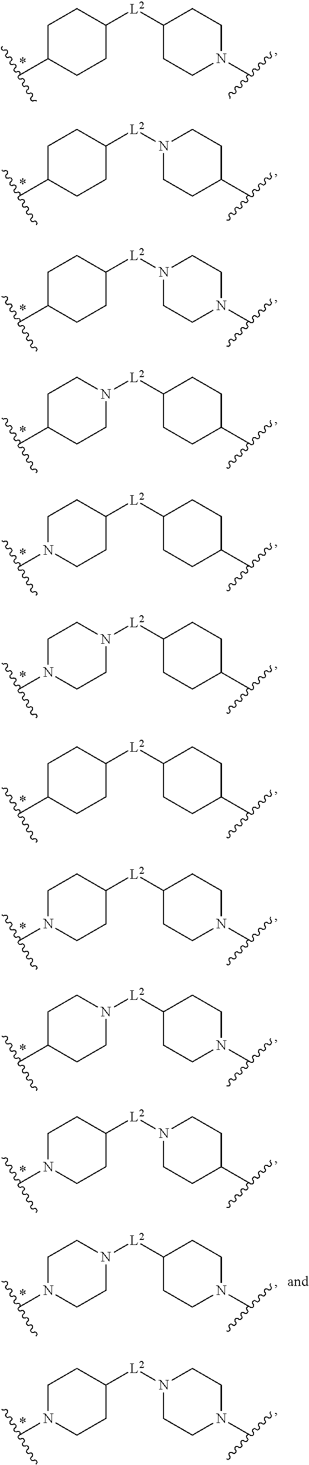

wherein each cyclohexyl, piperidinyl, and piperazinyl is substituted with 0-4 occurrences of R^{a}, wherein each R^{a} is independently selected from the group consisting of C₁₋₆ alkyl, C₁₋₆ alkoxyl, and halogen, or a pharmaceutically acceptable salt, hydrate, solvate, prodrug, stereoisomer, or tautomer thereof, wherein * denotes the point of attachment to L¹.

In an embodiment, X¹ and X² are each independently selected from the group consisting of piperidinyl and piperazinyl, wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen.

In an embodiment, X¹ and X² are both piperidinyl, wherein each piperidinyl is substituted with 0-4 occurrences of R^{a}, wherein each R^{a} is independently selected from the group consisting of C₁₋₆ alkyl, C₁₋₆ alkoxyl, and halogen.

In an embodiment, X¹-L²-X² is selected from the group consisting of

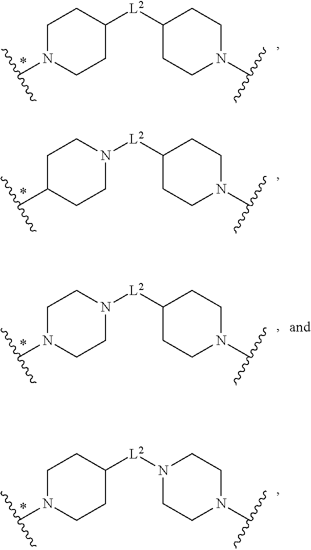

wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen, and wherein * denotes the point of attachment to L¹. In an embodiment, X¹-L²-X² is

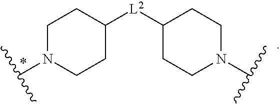

In an embodiment, L² is —CH₂—, O, or C₁₋₃ heteroalkylene. In an embodiment, L² is oxygen. In an embodiment, L² is —CH₂—.

In an embodiment, L¹ is —O— or C₁₋₆ alkylene.

In an embodiment, R⁴ is halogen, e.g., chloro or fluoro. In an embodiment, R⁴ is C₁₋₆ alkyl, e.g., methyl. In an embodiment, R⁴ is C₁₋₆ alkoxyl, e.g., methoxyl. In an embodiment, R⁴ is OH. In an embodiment, m is 0. In an embodiment, m is 1. In an embodiment, m is 2. In an embodiment, R⁴ is halogen, e.g., chloro, and m is 1. In an embodiment, R⁴ is C₁₋₆ alkyl, e.g., methyl, and m is 1. In an embodiment, R⁴ is C₁₋₆ alkoxyl, e.g., methoxyl, and m is 1. In an embodiment, R⁴ is OH, and m is 1.

In an embodiment, R^{d1} and R^{d2} are both methyl. In an embodiment, R^{d1} and R² are both H. In an embodiment, R^{d4} is H or C₁₋₆ alkyl, e.g., methyl. In an embodiment, R^{d5} is H or C₁₋₆ alkyl, e.g., methyl.

In another aspect, the compound is a compound of Formula (BF-III):

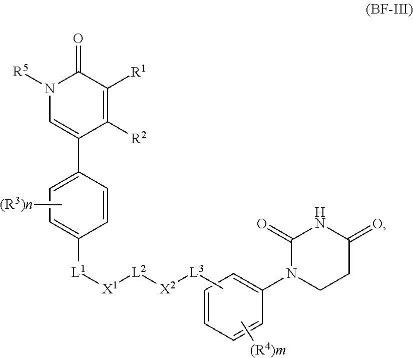

or a pharmaceutically acceptable salt, hydrate, solvate, prodrug, stereoisomer, or tautomer thereof, wherein:

R¹ and R² are independently selected from the group consisting of hydrogen and C₁₋₆ alkyl; or R¹ and R² together with the atoms to which they are attached form an aryl or heteroaryl;

R³ is selected from the group consisting of C₁₋₆ alkyl, C₁₋₆ alkoxyl, and halogen;

L¹ is selected from the group consisting of a bond, O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the phenyl ring in Formula (BF-III);

X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;

L² is selected from the group consisting of a bond, O, NR′, C₁₋₆ alkylene, and C₁₋₆ heteroalkylene; or

X¹-L²-X² form a spiroheterocyclyl;

L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O , wherein * denotes the point of attachment of L³ to X² in Formula (BF-III); wherein no more than 2 of L¹, X¹, X², L², and L³ can simultaneously be a bond;

R⁴ is selected from the group consisting of OH, C₁₋₆ alkyl, C₁₋₆ alkoxyl, and halogen;

R⁵ is selected from the group consisting of hydrogen and C₁₋₆ alkyl;

R′ is selected from the group consisting of hydrogen and C₁₋₆ alkyl; and

m and n are each independently 0, 1, or 2.

In an embodiment, the compound is a compound of Formula (BF-III), or a pharmaceutically acceptable salt, hydrate, solvate, prodrug, stereoisomer, or tautomer thereof,

wherein

R¹ and R² are independently selected from the group consisting of hydrogen and C₁₋₆ alkyl; or R¹ and R² together with the atoms to which they are attached form a heteroaryl;

R³ is selected from the group consisting of C₁₋₆ alkoxyl and halogen;

L¹ is selected from the group consisting of O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the phenyl ring in Formula (BF-III);

X¹-L²-X² is selected from the group consisting of:

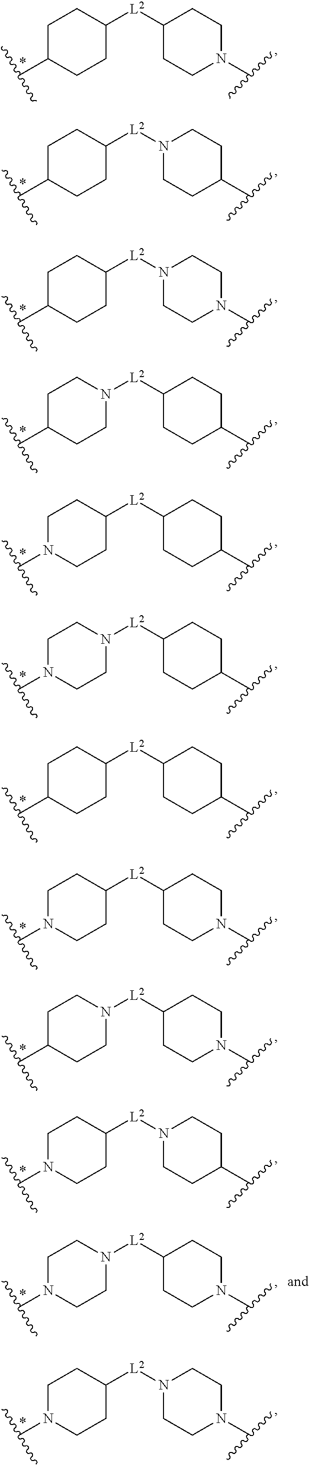

wherein * denotes the point of attachment to L¹, and wherein the carbocyclyl and heterocyclyl are substituted with 0-4 occurrences of R^{a}, wherein each R^{a} is halogen;

L² is selected from the group consisting of O, C₁₋₆ alkylene, and C₁₋₆ heteroalkylene;

L³ is selected from the group consisting of O, C(O), C₁₋₆ alkylene, C₁₋₆ heteroalkylene, and *C(O)—C₁₋₆ alkylene-O, wherein * denotes the point of attachment of L³ to X² in Formula (BF-III);

R⁴ is selected from the group consisting of OH, C₁₋₆ alkyl, C₁₋₆ alkoxyl, and halogen;

R⁵ is C₁₋₆ alkyl; and

m and n are each independently 0, 1, or 2.

In an embodiment, the compound is a compound of Formula (BF-III), or a pharmaceutically acceptable salt, hydrate, solvate, prodrug, stereoisomer, or tautomer thereof,

wherein

R¹ and R² are independently selected from the group consisting of hydrogen and methyl; or R¹ and R² together with the atoms to which they are attached form a pyridyl;

R³ is selected from the group consisting of methoxy, chloro, and fluoro;

L¹ is selected from the group consisting of O, C(O), C₁₋₃ alkylene, and C(O)—C₁₋₃ alkenylene*, wherein * denotes the point of attachment of L¹ to the phenyl ring in Formula (BF-III);

X¹-L²-X² is selected from the group consisting of:

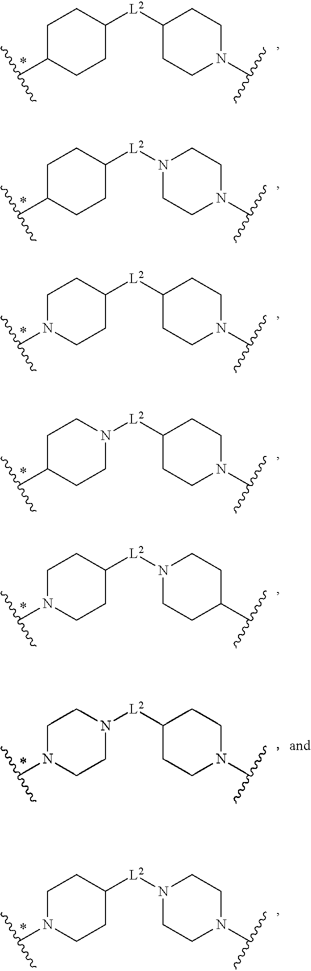

wherein * denotes the point of attachment to L¹, and wherein the carbocyclyl and heterocyclyl are substituted with 0-4 occurrences of R^{a}, wherein each R^{a} is fluoro;

L² is selected from the group consisting of C₁₋₃ alkylene, O, and C₁₋₃ heteroalkylene;

L³ is selected from the group consisting of O, C(O), C₁₋₃ alkylene, C₁₋₃ heteroalkylene, and *C(O)—C₁₋₃ alkylene-O, wherein * denotes the point of attachment of L³ to X² in Formula (BF-III);

R⁴ is selected from the group consisting of OH, methyl, methoxy, chloro, and fluoro;

R⁵ is C₁₋₆ alkyl; and

m and n are each independently 0, 1, or 2.

In an embodiment, the compound is a compound of Formula (BF-III), or a pharmaceutically acceptable salt, hydrate, solvate, prodrug, stereoisomer, or tautomer thereof,

wherein

R¹ and R² are methyl; or R¹ and R² together with the atoms to which they are attached form a pyridyl;

R³ is selected from the group consisting of methoxy, chloro, and fluoro;

L¹ is selected from the group consisting of O and C₁₋₃ alkylene;

X¹-L²-X² is selected from the group consisting of:

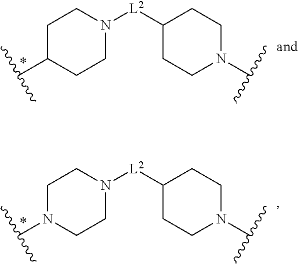

wherein * denotes the point of attachment to L¹, and wherein the heterocyclyl is substituted with 0 or 1 occurrences of R^{a}, wherein each R^{a} is fluoro;

L² is selected from the group consisting of C₁₋₃ alkylene and O;

L³ is selected from the group consisting of C(O) and C₁₋₃ heteroalkylene;

R⁴ is selected from the group consisting of methyl, methoxy, chloro, and fluoro;

R⁵ is C₃₋₆ alkyl; and

m and n are each independently 1 or 2.

In another aspect, the compound is a compound of Formula (BF-IIIa) or Formula (BF-IIIb):

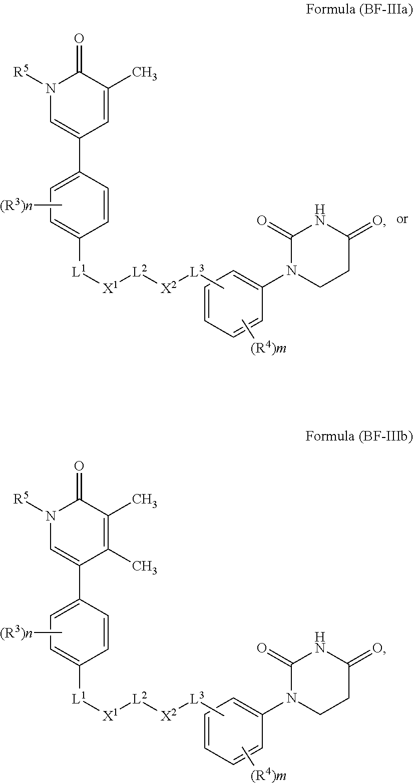

or a pharmaceutically acceptable salt, hydrate, solvate, prodrug, stereoisomer, or tautomer thereof.

In another aspect, the compound is a compound of Formula (BF-III′):

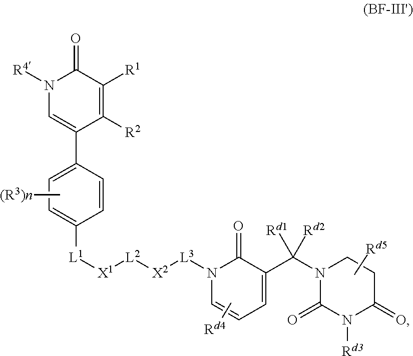

or a pharmaceutically acceptable salt, hydrate, solvate, prodrug, stereoisomer, or tautomer thereof, wherein:

R¹ and R² are independently selected from the group consisting of hydrogen and C₁₋₆ alkyl; or R¹ and R² together with the atoms to which they are attached form an aryl or heteroaryl;

R³ is selected from the group consisting of C₁₋₆ alkyl, C₁₋₆ alkoxyl, and halogen;

R^{4′} is selected from the group consisting of hydrogen and C₁₋₆ alkyl;

L¹ is selected from the group consisting of a bond, O, NR′, C(O), C₁₋₆ alkylene, C₁₋₆ heteroalkylene, *C(O)—C₁₋₆ alkylene, and *C(O)—C₁₋₆ heteroalkylene, wherein * denotes the point of attachment of L¹ to the phenyl ring in Formula (BF-III′);

X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;

L² is selected from the group consisting of a bond, O, NR′, C₁₋₆ alkylene, and C₁₋₆ heteroalkylene; or

X¹-L²-X² form a spiroheterocyclyl;

L³ is selected from the group consisting of a bond, C₁₋₆ alkylene, C₁₋₆ heteroalkylene, *C(O)—C₁₋₆ alkylene, and *C(O)—C₁₋₆ heteroalkylene, wherein * denotes the point of attachment of L³ to X² in Formula (BF-III′); wherein no more than 2 of L¹, X¹, X², L², and L³ can simultaneously be a bond;

R′ is selected from the group consisting of hydrogen and C₁₋₆ alkyl;

n is 0, 1, or 2;

R^{d1} and R^{d2} are each independently selected from the group consisting of H, C₁₋₆ alkyl, C₁₋₆ alkoxyl, C₁₋₆ haloalkyl, and C₁₋₆ heteroalkyl;

R^{d3} is H;

R^{d4} is selected from the group consisting of H, C₁₋₆ alkyl, halo, C₁₋₆ haloalkyl, and C₁₋₆ heteroalkyl; and

R^{d5} is selected from the group consisting of H, C₁₋₆ alkyl, halo, C₁₋₆ haloalkyl, and C₁₋₆ heteroalkyl.

In an embodiment, the compound is a compound of Formula (BF-III′), or a pharmaceutically acceptable salt, hydrate, solvate, prodrug, stereoisomer, or tautomer thereof,

wherein

R¹ and R² are independently selected from the group consisting of hydrogen and C₁₋₆ alkyl; or R¹ and R² together with the atoms to which they are attached form a heteroaryl;

R³ is selected from the group consisting of C₁₋₆ alkoxyl and halogen;

R^{4′} is C₁₋₆ alkyl;

L¹ is selected from the group consisting of O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the phenyl ring in Formula (BF-III′);

X¹-L²-X² is selected from the group consisting of:

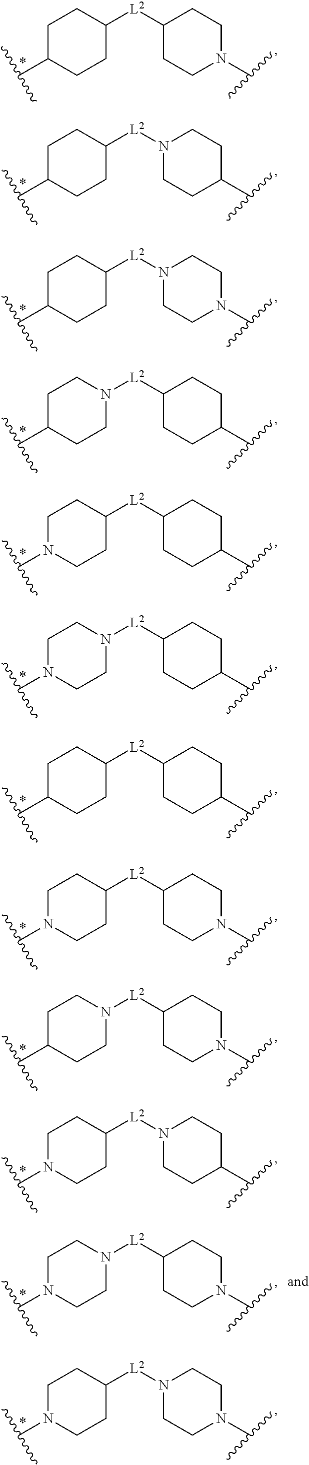

wherein * denotes the point of attachment to L¹, and wherein the carbocyclyl and heterocyclyl are substituted with 0-4 occurrences of R^{a}, wherein each R^{a} is halogen;

L² is selected from the group consisting of O, C₁₋₆ alkylene, and C₁₋₆ heteroalkylene; or

L³ is selected from the group consisting of C₁₋₆ alkylene and C₁₋₆ heteroalkylene, wherein * denotes the point of attachment of L³ to X² in Formula (BF-III′);

n is 0, 1, or 2;

R^{d1} and R^{d2} are each independently selected from the group consisting of H and C₁₋₆ alkyl;

R^{d3} is H;

R^{d4} is selected from the group consisting of H, C₁₋₆ alkyl, and halogen; and

R^{d5} is selected from the group consisting of H and C₁₋₆ alkyl.

In an embodiment of the compounds disclosed herein, R¹ and R² together with the atoms to which they are attached form an aryl or heteroaryl. In an embodiment, R¹ and R² together with the atoms to which they are attached form a phenyl. In an embodiment, R¹ and R² together with the atoms to which they are attached form a heteroaryl. In an embodiment, R¹ and R² together with the atoms to which they are attached form a 5- or 6-membered heteroaryl. In an embodiment, R¹ and R² together with the atoms to which they are attached form a 6-membered heteroaryl. In an embodiment, R¹ and R² together with the atoms to which they are attached form a 6-membered nitrogen-containing heteroaryl. In an embodiment, R¹ and R² together with the atoms to which they are attached form a pyridyl.

In an embodiment, R³ is selected from the group consisting of methoxyl, chloro, and fluoro. In an embodiment, R³ is methoxyl. In an embodiment, R³ is chloro or fluoro.

In an embodiment, n is 1 or 2.

In an embodiment, R³ is methoxyl and n is 1 or 2. In an embodiment, n is 1. In an embodiment, n is 2. In an embodiment, n is 0.

In an embodiment, one of X¹ and X² is not a bond. In an embodiment, one of X¹ and X² is a bond and the other is a carbocyclyl or heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen. In an embodiment, one of X¹ and X² is a bond and the other is a heterocyclyl, wherein the heterocyclyl is substituted with 0-4 occurrences of R^{a}, wherein each R^{a} is independently selected from the group consisting of C₁₋₆ alkyl, C₁₋₆ alkoxyl, and halogen.

In an embodiment, X¹ and X² are each independently selected from the group consisting of cyclohexyl, piperidinyl, and piperazinyl, wherein each cyclohexyl, piperidinyl, and piperazinyl is substituted with 0-4 occurrences of R^{a}, wherein each R^{a} is independently selected from the group consisting of C₁₋₆ alkyl, C₁₋₆ alkoxyl, and halogen.

In an embodiment, —X¹-L²-X²— is selected from the group

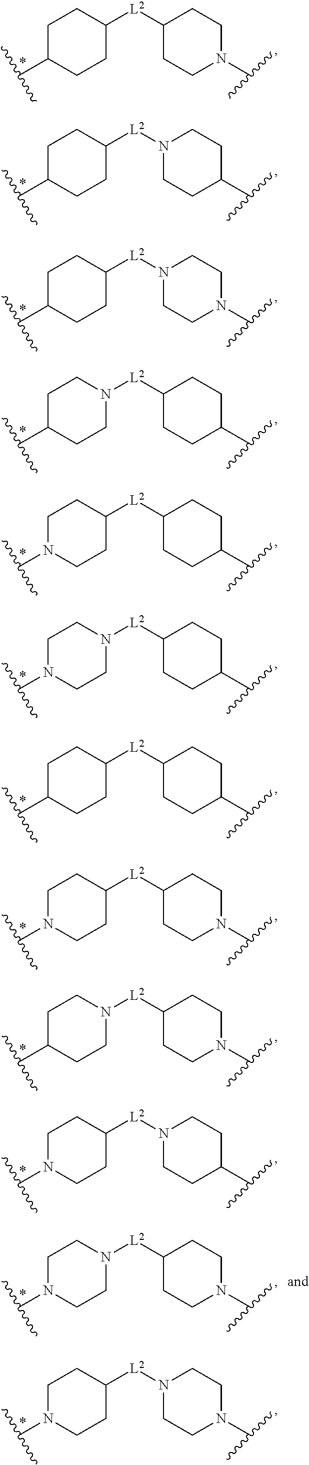

wherein each cyclohexyl, piperidinyl, and piperazinyl is substituted with 0-4 occurrences of R^{a}, wherein each R^{a} is independently selected from the group consisting of C₁₋₆ alkyl, C₁₋₆ alkoxyl, and halogen, or a pharmaceutically acceptable salt, hydrate, solvate, prodrug, stereoisomer, or tautomer thereof, wherein * denotes the point of attachment to L¹.

In an embodiment, X¹ and X² are each independently selected from the group consisting of piperidinyl and piperazinyl, wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen.

In an embodiment, X¹ and X² are both piperidinyl, wherein each piperidinyl is substituted with 0-4 occurrences of R^{a}, wherein each R^{a} is independently selected from the group consisting of C₁₋₆ alkyl, C₁₋₆ alkoxyl, and halogen.

In an embodiment, X¹-L²-X² is selected from the group consisting of:

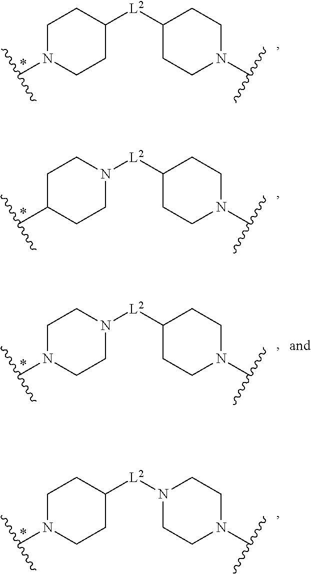

wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen, and wherein * denotes the point of attachment to L¹. In an embodiment, X¹-L²-X² is

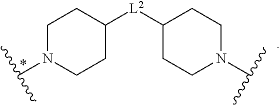

In an embodiment, L² is selected from the group consisting of O, C₁₋₆ alkylene, and C₁₋₆ heteroalkylene. In an embodiment, L² is —CH₂—, O, or C₁₋₃ heteroalkylene. In an embodiment, L² is oxygen. In an embodiment, L₂ is —CH₂—.

In an embodiment, each R^{a} is halogen. In an embodiment, each R^{a} is fluoro.

In an embodiment, L¹ is —O— or C₁₋₆ alkylene.

In an embodiment, R⁴ is halogen, e.g., chloro or fluoro. In an embodiment, R⁴ is C₁₋₆ alkyl, e.g., methyl. In an embodiment, R⁴ is C₁₋₆ alkoxyl, e.g., methoxyl. In an embodiment, R⁴ is OH. In an embodiment, m is 0. In an embodiment, m is 1. In an embodiment, m is 2. In an embodiment, R⁴ is halogen, e.g., chloro, and m is 1. In an embodiment, R⁴ is C₁₋₆ alkyl, e.g., methyl, and m is 1. In an embodiment, R⁴ is C₁₋₆ alkoxyl, e.g., methoxyl, and m is 1. In an embodiment, R⁴ is OH, and m is 1.

In an embodiment, R⁵ is methyl. In an embodiment, R⁵ is n-butyl.

In an embodiment, R^{4′} is methyl. In an embodiment, R^{4′} is n-butyl.

In an embodiment, R^{d1} and R^{d2} are both methyl. In an embodiment, R^{d1} and R^{d2} are both H. In an embodiment, R^{d4} is H or C₁₋₆ alkyl, e.g., methyl. In an embodiment, R^{d5} is H or C₁₋₆ alkyl, e.g., methyl.

In another aspect, the compound is a compound of Formula (BF-IV):

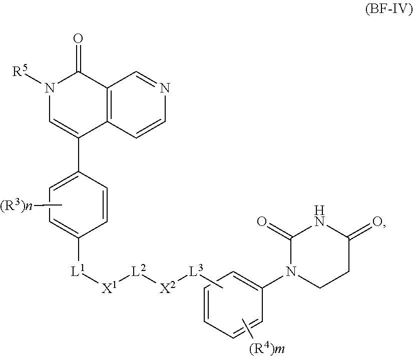

or a pharmaceutically acceptable salt, hydrate, solvate, prodrug, stereoisomer, or tautomer thereof, wherein:

R³ is selected from the group consisting of C₁₋₆ alkyl, C₁₋₆ alkoxyl, and halogen;

L¹ is selected from the group consisting of a bond, O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the phenyl ring in Formula (BF-IV);

X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;

L² is selected from the group consisting of a bond, O, NR′, C₁₋₆ alkylene, and C₁₋₆ heteroalkylene; or

X¹-L²-X² form a spiroheterocyclyl;

L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O , wherein * denotes the point of attachment of L³ to X² in Formula (BF-IV); wherein no more than 2 of L¹, X¹, X², L², and L³ can simultaneously be a bond;

R⁴ is selected from the group consisting of OH, C₁₋₆ alkyl, C₁₋₆ alkoxyl, and halogen;

R⁵ is selected from the group consisting of hydrogen and C₁₋₆ alkyl;

R′ is selected from the group consisting of hydrogen and C₁₋₆ alkyl; and

m and n are each independently 0, 1, or 2.

In another aspect, the compound is a compound of Formula (BF-IVa) or (BF-IVb):

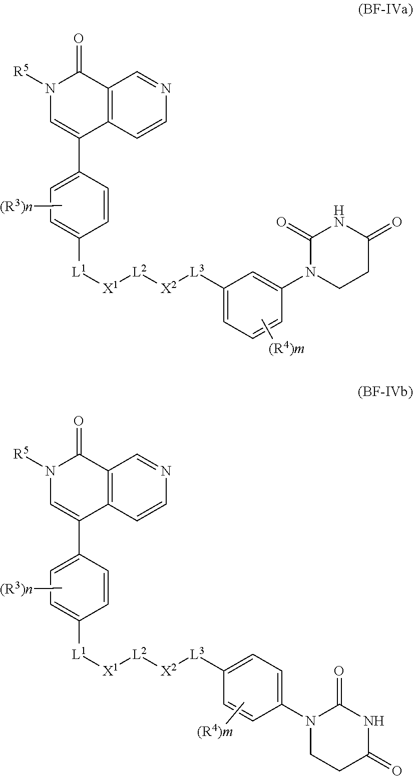

or a pharmaceutically acceptable salt, hydrate, solvate, prodrug, stereoisomer, or tautomer thereof.

In another aspect, the compound is a compound of Formula (BF-IV′):

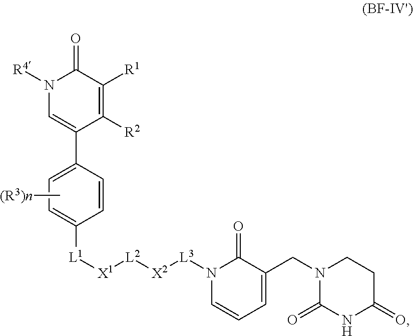

or a pharmaceutically acceptable salt, hydrate, solvate, prodrug, stereoisomer, or tautomer thereof, wherein:

R¹ and R² are independently selected from the group consisting of hydrogen and C₁₋₆ alkyl; or R¹ and R² together with the atoms to which they are attached form an aryl or heteroaryl;

R³ is selected from the group consisting of C₁₋₆ alkyl, C₁₋₆ alkoxyl, and halogen;

R^{4′} is selected from the group consisting of hydrogen or C₁₋₆ alkyl;

L¹ is selected from the group consisting of a bond, O, NR′, C(O), C₁₋₆ alkylene, C₁₋₆ heteroalkylene, *C(O)—C₁₋₆ alkylene, and *C(O)—C₁₋₆ heteroalkylene, wherein * denotes the point of attachment of L¹ to the phenyl ring in Formula (BF-IV′);

X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;

L² is selected from the group consisting of a bond, O, NR′, C₁₋₆ alkylene, and C₁₋₆ heteroalkylene; or

X¹-L²-X² form a spiroheterocyclyl;

L³ is selected from the group consisting of a bond, C₁₋₆ alkylene, C₁₋₆ heteroalkylene, *C(O)—C₁₋₆ alkylene, and *C(O)—C₁₋₆ heteroalkylene, wherein * denotes the point of attachment of L³ to X² in Formula (BF-IV′); wherein no more than 2 of L¹, X¹, X², L², and L³ can simultaneously be a bond;

R′ is selected from the group consisting of hydrogen and C₁₋₆ alkyl; and

n is 0, 1, or 2.

In an embodiment, the compound is a compound of Formula (BF-IV′), or a pharmaceutically acceptable salt, hydrate, solvate, prodrug, stereoisomer, or tautomer thereof,

wherein

R¹ and R² are C₁₋₆ alkyl; or R¹ and R² together with the atoms to which they are attached form a heteroaryl;

R³ is selected from the group consisting of C₁₋₆ alkoxyl and halogen;

R⁴ is C₁₋₆ alkyl;

L¹ is selected from the group consisting of O and C₁₋₆ alkylene;

X¹-L²-X² is selected from the group consisting of:

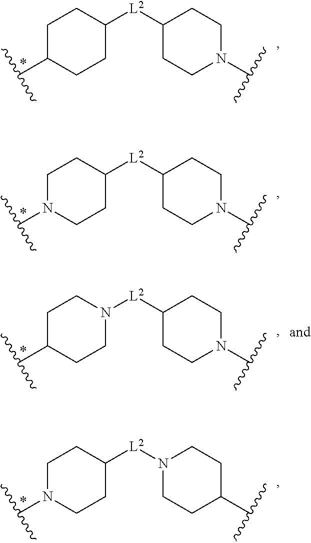

wherein * denotes the point of attachment to L¹, and wherein the carbocyclyl and heterocyclyl are substituted with 0-4 occurrences of R^{a}, wherein each R^{a} is halogen;

L² is selected from the group consisting of O and C₁₋₆ alkylene;

L³ is C₁₋₆ alkylene; and

n is 0, 1, or 2.

In an embodiment, the compound is a compound of Formula (BF-IV′), or a pharmaceutically acceptable salt, hydrate, solvate, prodrug, stereoisomer, or tautomer thereof,

wherein

R¹ and R² are methyl; or R¹ and R² together with the atoms to which they are attached form a pyridyl;

R³ is selected from the group consisting of methoxy, chloro, and fluoro;

R^{4′} is C₁₋₆ alkyl;

L¹ is selected from the group consisting of O and C₁₋₃ alkylene;

X¹-L²-X² is selected from the group consisting of:

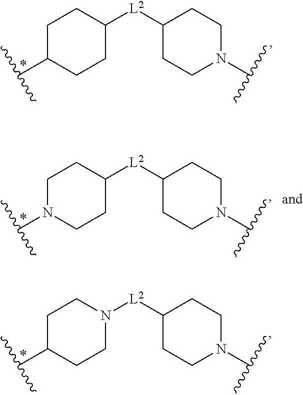

wherein * denotes the point of attachment to L¹, and wherein the carbocyclyl and heterocyclyl are substituted with 0-4 occurrences of R^{a}, wherein each R^{a} is fluoro;

L² is selected from the group consisting of O and C₁₋₃ alkylene;

L³ is C₂₋₃ alkylene;

n is 0, 1, or 2.

In another aspect, the compound is a compound of Formula (BF-V′):

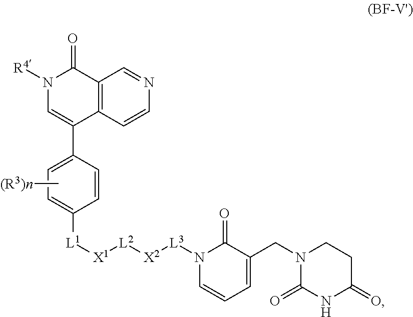

or a pharmaceutically acceptable salt, hydrate, solvate, prodrug, stereoisomer, or tautomer thereof, wherein:

R³ is selected from the group consisting of C₁₋₆ alkyl, C₁₋₆ alkoxyl, and halogen;

R^{4′} is selected from the group consisting of hydrogen or C₁₋₆ alkyl;

L¹ is selected from the group consisting of a bond, NR′, C₁₋₆ alkylene, C₁₋₆ heteroalkylene, and *C(O)—C₁₋₆ alkylene, wherein * denotes the point of attachment of L¹ to the phenyl ring in Formula (BF-V′);

X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;

L² is selected from the group consisting of a bond, O, NR′, C₁₋₆ alkylene, and C₁₋₆ heteroalkylene; or

X¹-L²-X² form a spiroheterocyclyl;

L³ is selected from the group consisting of a bond, C₁₋₆ alkylene, C₁₋₆ heteroalkylene, *C(O)—C₁₋₆ alkylene, and *C(O)—C₁₋₆ heteroalkylene, wherein * denotes the point of attachment of L³ to X² in Formula (BF-V′); wherein no more than 2 of L¹, X¹, X², L², and L³ can simultaneously be a bond;

R′ is selected from the group consisting of hydrogen and C₁₋₆ alkyl; and

n is 0, 1, or 2.

In an embodiment, R³ is independently selected from the group consisting of methoxyl, chloro, and fluoro. In an embodiment, R³ is independently selected from the group consisting of methoxyl and chloro. In an embodiment, R³ is methoxyl. In an embodiment, R³ is chloro or fluoro.

In an embodiment, n is 1 or 2.

In an embodiment, R³ is methoxyl and n is 1 or 2. In an embodiment, n is 1. In an embodiment, n is 2. In an embodiment, n is 0.

In an embodiment, one of X¹ and X² is not a bond. In an embodiment, one of X¹ and X² is a bond and the other is a carbocyclyl or heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen. In an embodiment, one of X¹ and X² is a bond and the other is a heterocyclyl, wherein the heterocyclyl is substituted with 0-4 occurrences of R^{a}, wherein each R^{a} is independently selected from the group consisting of C₁₋₆ alkyl, C₁₋₆ alkoxyl, and halogen.

In an embodiment, X¹ and X² are each independently selected from the group consisting of cyclohexyl, piperidinyl, and piperazinyl, wherein each cyclohexyl, piperidinyl, and piperazinyl is substituted with 0-4 occurrences of R^{a}, wherein each R^{a} is independently selected from the group consisting of C₁₋₆ alkyl, C₁₋₆ alkoxyl, and halogen.

In an embodiment —X¹-L²-X²— is selected from the group consisting of

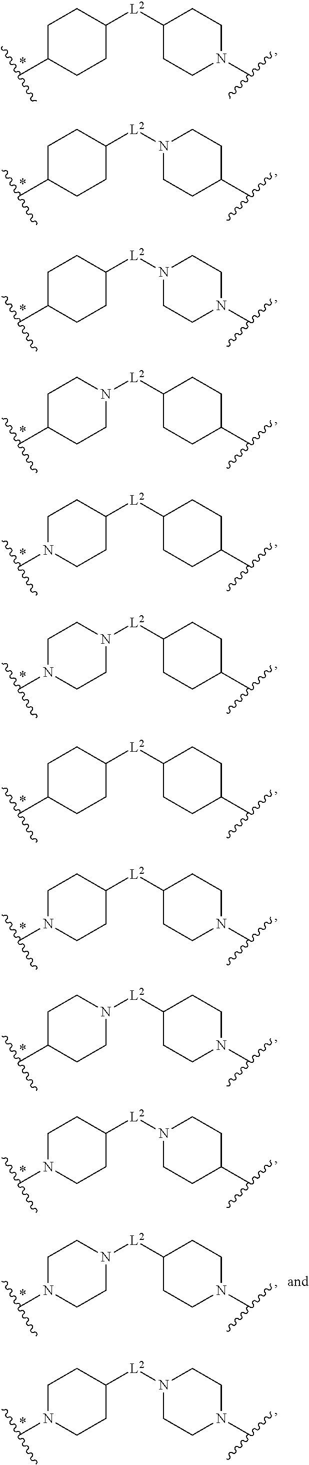

wherein each cyclohexyl, piperidinyl, and piperazinyl is substituted with 0-4 occurrences of R^{a}, wherein each R^{a} is independently selected from the group consisting of C₁₋₆ alkyl, C₁₋₆ alkoxyl, and halogen, or a pharmaceutically acceptable salt, hydrate, solvate, prodrug, stereoisomer, or tautomer thereof, wherein * denotes the point of attachment to L¹.

In an embodiment, X¹ and X² are each independently selected from the group consisting of piperidinyl and piperazinyl, wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen.

In an embodiment, X¹ and X² are both piperidinyl, wherein each piperidinyl is substituted with 0-4 occurrences of R^{a}, wherein each R^{a} is independently selected from the group consisting of C₁₋₆ alkyl, C₁₋₆ alkoxyl, and halogen.

In an embodiment, X¹-L²-X² is selected from the group consisting of:

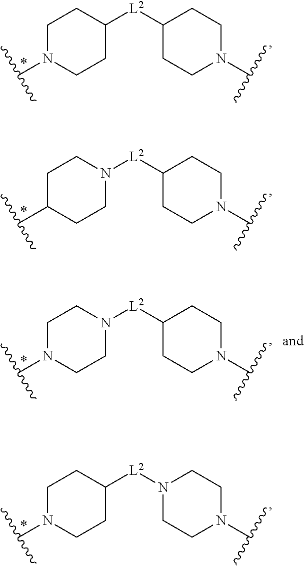

wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen, and wherein * denotes the point of attachment to L¹. In an embodiment, X¹-L²-X² is

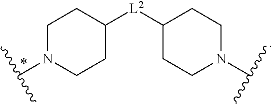

In an embodiment, L² is selected from the group consisting of O, C₁₋₆ alkylene, and C₁₋₆ heteroalkylene. In an embodiment, L² is —CH₂—, O, or C₁₋₃ heteroalkylene. In an embodiment, L² is oxygen. In an embodiment, L₂ is —CH₂—.

In an embodiment, each R^{a} is halogen. In an embodiment, each R^{a} is fluoro.

In an embodiment, L¹ is —O— or C₁₋₆ alkylene.

In an embodiment, R⁴ is halogen, e.g., chloro or fluoro. In an embodiment, R⁴ is C₁₋₆ alkyl, e.g., methyl. In an embodiment, R⁴ is C₁₋₆ alkoxyl, e.g., methoxyl. In an embodiment, R⁴ is OH. In an embodiment, m is 0. In an embodiment, m is 1. In an embodiment, m is 2. In an embodiment, R⁴ is halogen, e.g., chloro, and m is 1. In an embodiment, R⁴ is C₁₋₆ alkyl, e.g., methyl, and m is 1. In an embodiment, R⁴ is C₁₋₆ alkoxyl, e.g., methoxyl, and m is 1. In an embodiment, R⁴ is OH, and m is 1.

In an embodiment, R⁵ is methyl. In an embodiment, R⁵ is n-butyl.

In an embodiment, R^{4′} is methyl. In an embodiment, R^{4′} is n-butyl.

In an embodiment, the compound of a Formula above or a pharmaceutically acceptable salt, hydrate, solvate, prodrug, stereoisomer, or tautomer thereof, is selected from the group consisting of:

| Compound | |
|---|---|
| Number | Compound Structure |
| A1 | 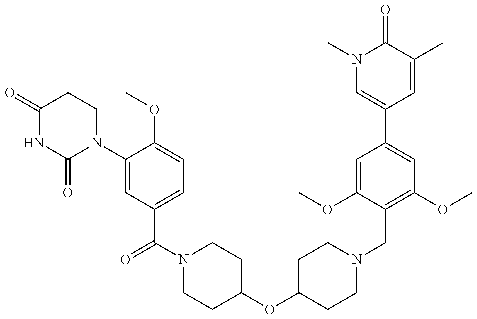 [SMILES: COc1ccc(C(=O)N2CCC(OC3CCN(Cc4c(OC)cc(-c5cc(C)c(=O)n(C)c5)cc4OC)CC3)CC2)cc1N1CCC(=O)NC1=O] |
| A2 | 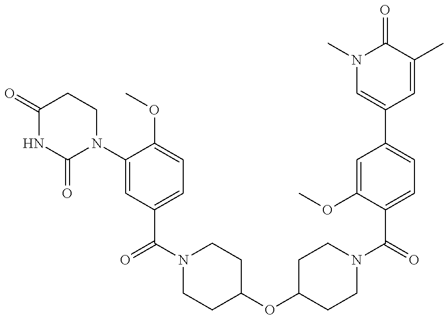 [SMILES: COc1cc(-c2cc(C)c(=O)n(C)c2)ccc1C(=O)N1CCC(OC2CCN(C(=O)c3ccc(OC)c(N4CCC(=O)NC4=O)c3)CC2)CC1] |
| A3 | 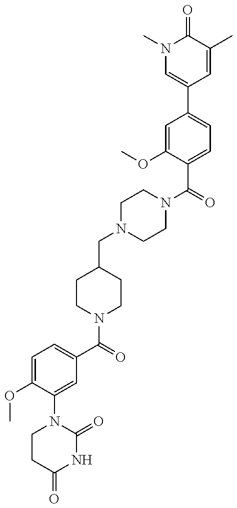 [SMILES: COc1cc(-c2cc(C)c(=O)n(C)c2)ccc1C(=O)N1CCN(CC2CCN(C(=O)c3ccc(OC)c(N4CCC(=O)NC4=O)c3)CC2)CC1] |
| A4 | 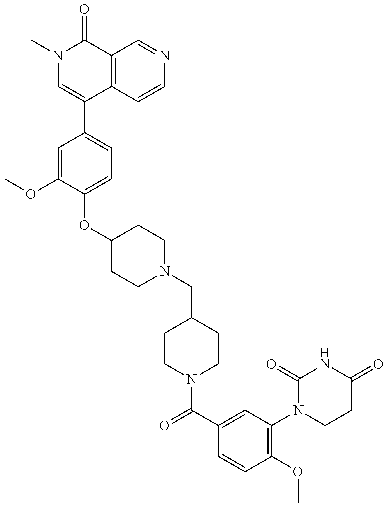 [SMILES: COc1cc(-c2cn(C)c(=O)c3cnccc23)ccc1OC1CCN(CC2CCN(C(=O)c3ccc(OC)c(N4CCC(=O)NC4=O)c3)CC2)CC1] |
| A5 | 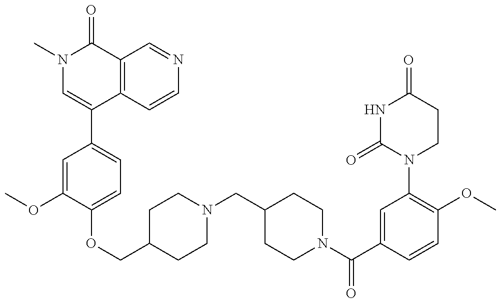 |
| A6 | 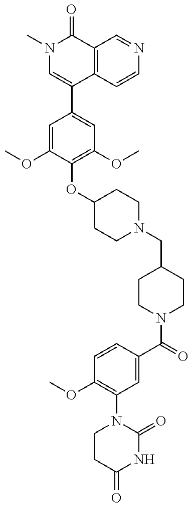 [SMILES: COc1ccc(C(=O)N2CCC(CN3CCC(Oc4c(OC)cc(-c5cn(C)c(=O)c6cnccc56)cc4OC)CC3)CC2)cc1N1CCC(=O)NC1=O] |
| A7 | 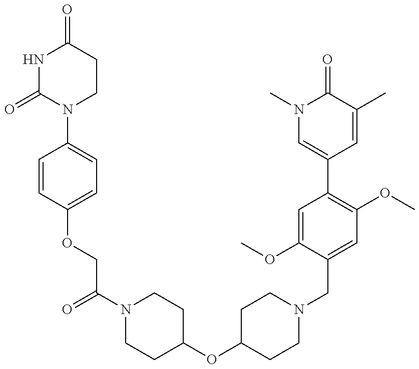 [SMILES: COc1cc(-c2cc(C)c(=O)n(C)c2)c(OC)cc1CN1CCC(OC2CCN(C(=O)COc3ccc(N4CCC(=O)NC4=O)cc3)CC2)CC1] |
| A8 | 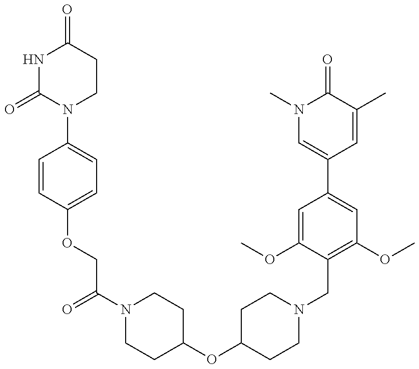 [SMILES: COc1cc(-c2cc(C)c(=O)n(C)c2)cc(OC)c1CN1CCC(OC2CCN(C(=O)COc3ccc(N4CCC(=O)NC4=O)cc3)CC2)CC1] |
| A9 | 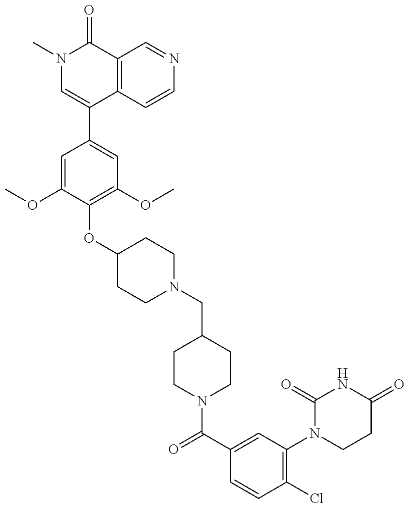 [SMILES: COc1cc(-c2cn(C)c(=O)c3cnccc23)cc(OC)c1OC1CCN(CC2CCN(C(=O)c3ccc(Cl)c(N4CCC(=O)NC4=O)c3)CC2)CC1] |
| A10 | 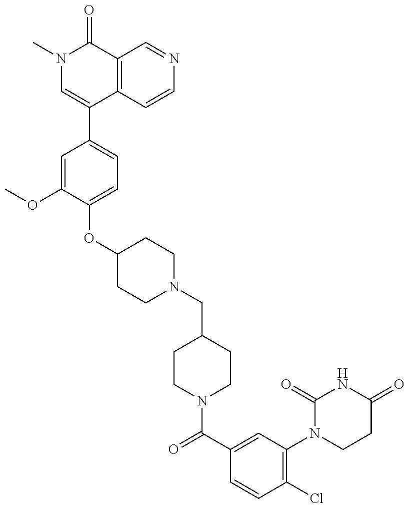 [SMILES: COc1cc(-c2cn(C)c(=O)c3cnccc23)ccc1OC1CCN(CC2CCN(C(=O)c3ccc(Cl)c(N4CCC(=O)NC4=O)c3)CC2)CC1] |
| A11 | 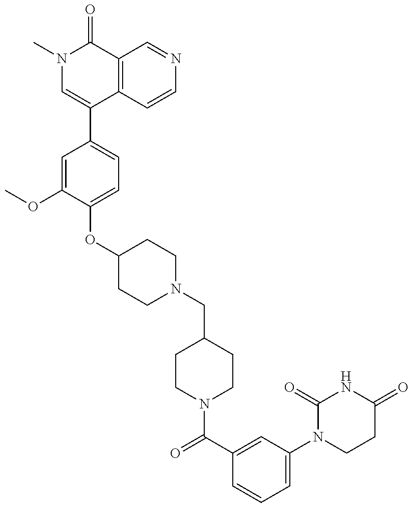 [SMILES: COc1cc(-c2cn(C)c(=O)c3cnccc23)ccc1OC1CCN(CC2CCN(C(=O)c3cccc(N4CCC(=O)NC4=O)c3)CC2)CC1] |
| A12 | 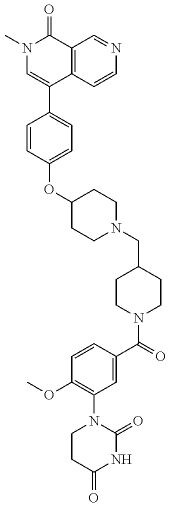 [SMILES: COc1ccc(C(=O)N2CCC(CN3CCC(Oc4ccc(-c5cn(C)c(=O)c6cnccc56)cc4)CC3)CC2)cc1N1CCC(=O)NC1=O] |
| A13 | 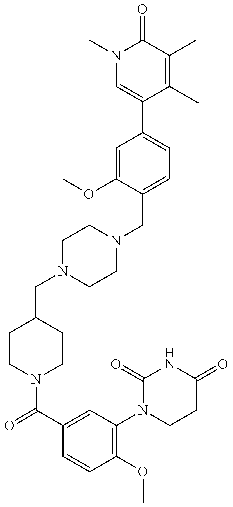 [SMILES: COc1cc(-c2cn(C)c(=O)c(C)c2C)ccc1CN1CCN(CC2CCN(C(=O)c3ccc(OC)c(N4CCC(=O)NC4=O)c3)CC2)CC1] |
| A14 | 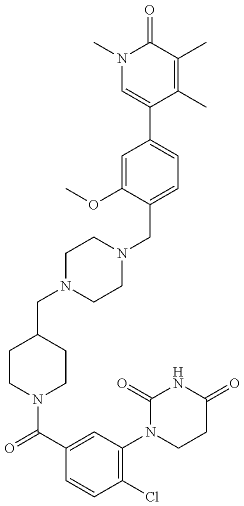 [SMILES: COc1cc(-c2cn(C)c(=O)c(C)c2C)ccc1CN1CCN(CC2CCN(C(=O)c3ccc(Cl)c(N4CCC(=O)NC4=O)c3)CC2)CC1] |
| A15 | 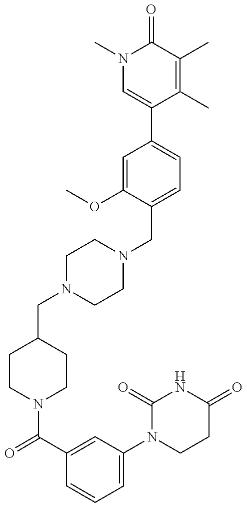 [SMILES: COc1cc(-c2cn(C)c(=O)c(C)c2C)ccc1CN1CCN(CC2CCN(C(=O)c3cccc(N4CCC(=O)NC4=O)c3)CC2)CC1] |
| A16 | 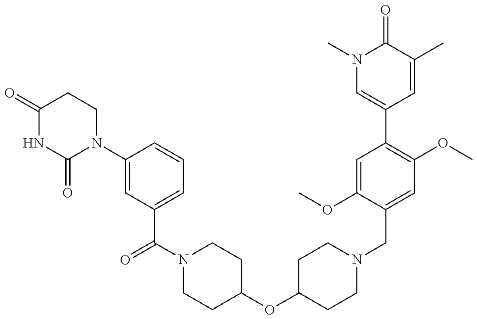 [SMILES: COc1cc(-c2cc(C)c(=O)n(C)c2)c(OC)cc1CN1CCC(OC2CCN(C(=O)c3cccc(N4CCC(=O)NC4=O)c3)CC2)CC1] |
| A17 | 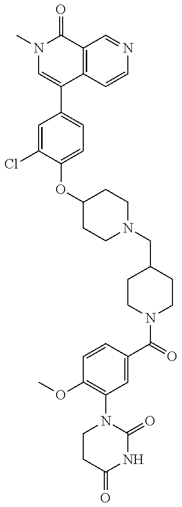 [SMILES: COc1ccc(C(=O)N2CCC(CN3CCC(Oc4ccc(-c5cn(C)c(=O)c6cnccc56)cc4Cl)CC3)CC2)cc1N1CCC(=O)NC1=O] |
| A18 | 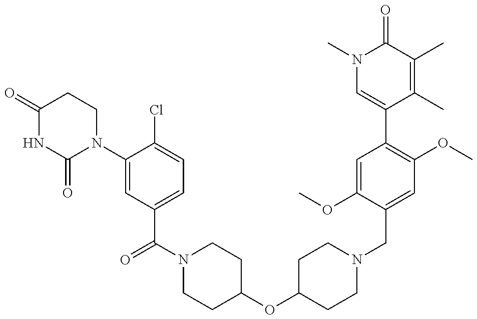 [SMILES: COc1cc(-c2cn(C)c(=O)c(C)c2C)c(OC)cc1CN1CCC(OC2CCN(C(=O)c3ccc(Cl)c(N4CCC(=O)NC4=O)c3)CC2)CC1] |
| A19 | 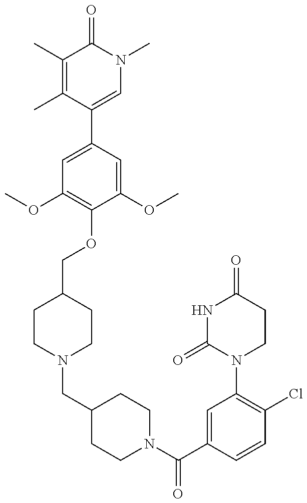 [SMILES: COc1cc(-c2cn(C)c(=O)c(C)c2C)cc(OC)c1OCC1CCN(CC2CCN(C(=O)c3ccc(Cl)c(N4CCC(=O)NC4=O)c3)CC2)CC1] |
| A20 | 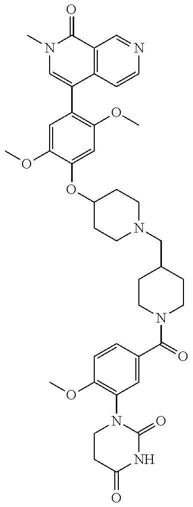 [SMILES: COc1cc(-c2cn(C)c(=O)c3cnccc23)c(OC)cc1OC1CCN(CC2CCN(C(=O)c3ccc(OC)c(N4CCC(=O)NC4=O)c3)CC2)CC1] |
| A21 | 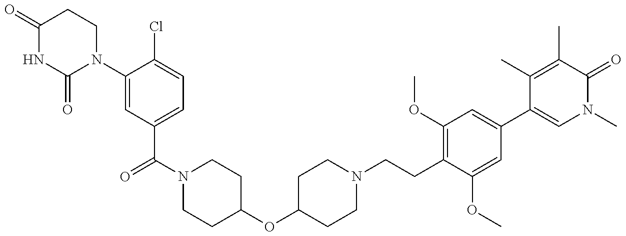 [SMILES: COc1cc(-c2cn(C)c(=O)c(C)c2C)cc(OC)c1CCN1CCC(OC2CCN(C(=O)c3ccc(Cl)c(N4CCC(=O)NC4=O)c3)CC2)CC1] |
| A22 | 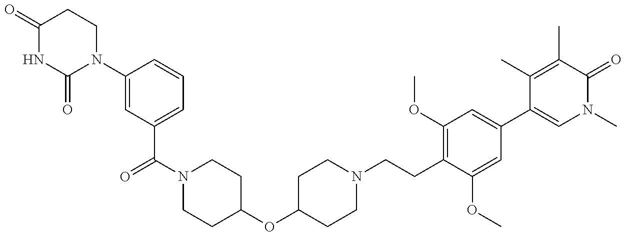 [SMILES: COc1cc(-c2cn(C)c(=O)c(C)c2C)cc(OC)c1CCN1CCC(OC2CCN(C(=O)c3cccc(N4CCC(=O)NC4=O)c3)CC2)CC1] |
| A23 | 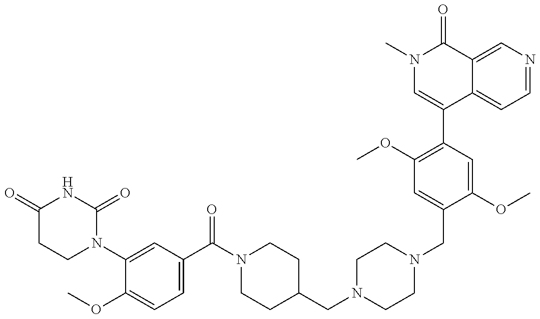 [SMILES: COc1cc(-c2cn(C)c(=O)c3cnccc23)c(OC)cc1CN1CCN(CC2CCN(C(=O)c3ccc(OC)c(N4CCC(=O)NC4=O)c3)CC2)CC1] |
| A24 | 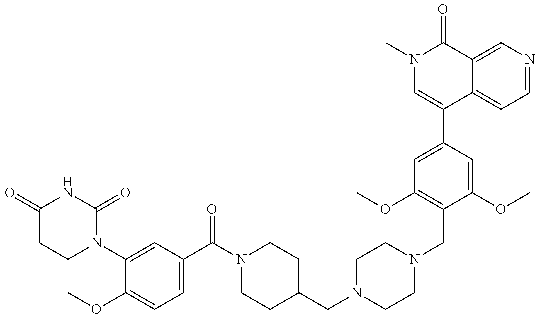 [SMILES: COc1ccc(C(=O)N2CCC(CN3CCN(Cc4c(OC)cc(-c5cn(C)c(=O)c6cnccc56)cc4OC)CC3)CC2)cc1N1CCC(=O)NC1=O] |
| A25 | 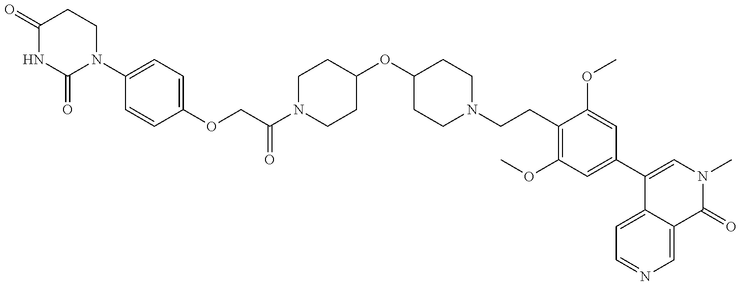 [SMILES: COc1cc(-c2cn(C)c(=O)c3cnccc23)cc(OC)c1CCN1CCC(OC2CCN(C(=O)COc3ccc(N4CCC(=O)NC4=O)cc3)CC2)CC1] |
| A26 | 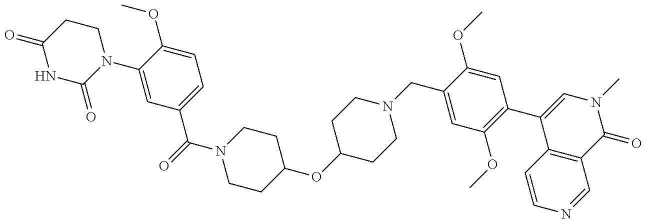 [SMILES: COc1cc(-c2cn(C)c(=O)c3cnccc23)c(OC)cc1CN1CCC(OC2CCN(C(=O)c3ccc(OC)c(N4CCC(=O)NC4=O)c3)CC2)CC1] |
| A27 | 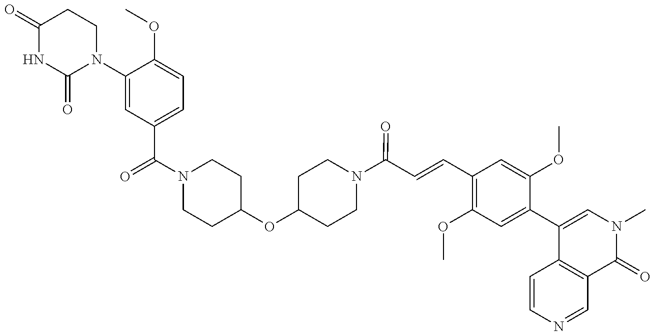 [SMILES: COc1cc(-c2cn(C)c(=O)c3cnccc23)c(OC)cc1/C=C/C(=O)N1CCC(OC2CCN(C(=O)c3ccc(OC)c(N4CCC(=O)NC4=O)c3)CC2)CC1] |
| A28 | 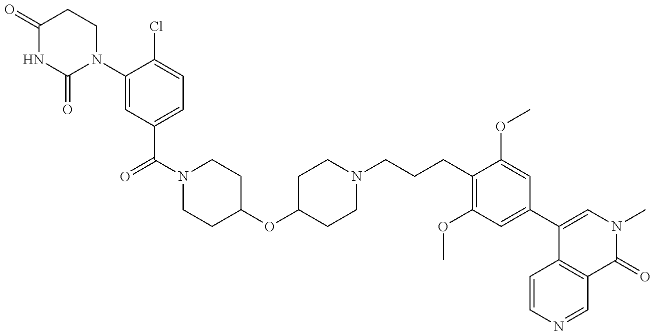 [SMILES: COc1cc(-c2cn(C)c(=O)c3cnccc23)cc(OC)c1CCCN1CCC(OC2CCN(C(=O)c3ccc(Cl)c(N4CCC(=O)NC4=O)c3)CC2)CC1] |
| A29 | 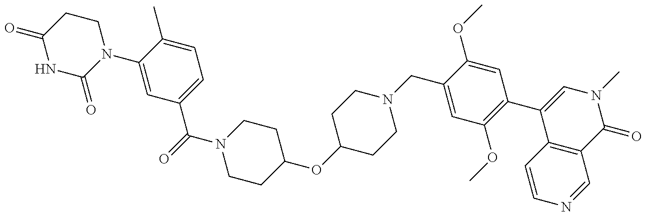 [SMILES: COc1cc(-c2cn(C)c(=O)c3cnccc23)c(OC)cc1CN1CCC(OC2CCN(C(=O)c3ccc(C)c(N4CCC(=O)NC4=O)c3)CC2)CC1] |
| A30 | 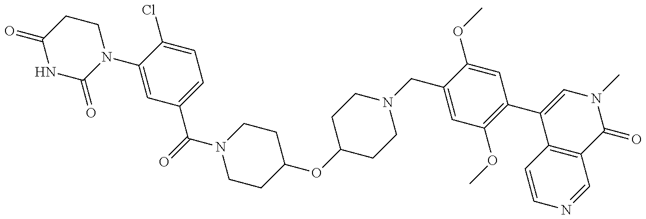 [SMILES: COc1cc(-c2cn(C)c(=O)c3cnccc23)c(OC)cc1CN1CCC(OC2CCN(C(=O)c3ccc(Cl)c(N4CCC(=O)NC4=O)c3)CC2)CC1] |
| A31 | 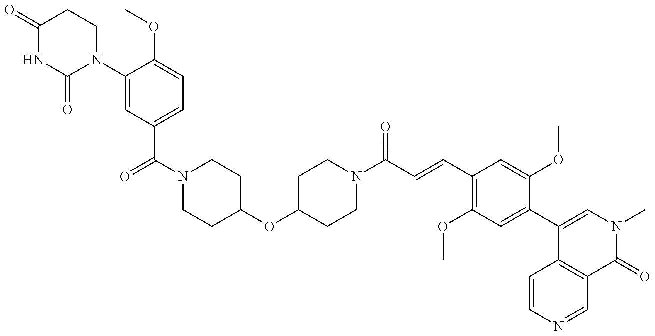 [SMILES: COc1cc(-c2cn(C)c(=O)c3cnccc23)c(OC)cc1/C=C/C(=O)N1CCC(OC2CCN(C(=O)c3ccc(OC)c(N4CCC(=O)NC4=O)c3)CC2)CC1] |
| A32 | 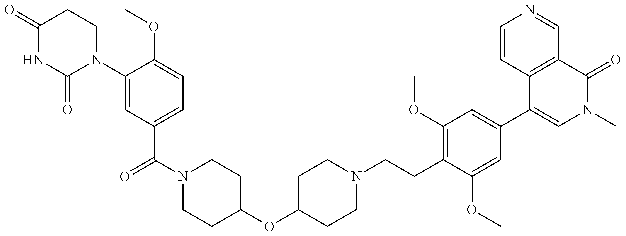 [SMILES: COc1ccc(C(=O)N2CCC(OC3CCN(CCc4c(OC)cc(-c5cn(C)c(=O)c6cnccc56)cc4OC)CC3)CC2)cc1N1CCC(=O)NC1=O] |
| A33 | 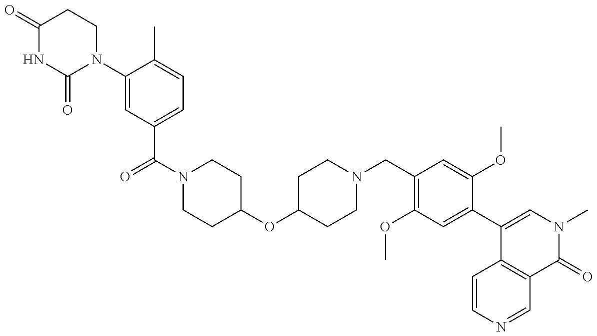 [SMILES: COc1cc(-c2cn(C)c(=O)c3cnccc23)c(OC)cc1CN1CCC(OC2CCN(C(=O)c3ccc(C)c(N4CCC(=O)NC4=O)c3)CC2)CC1] |
| A34 | 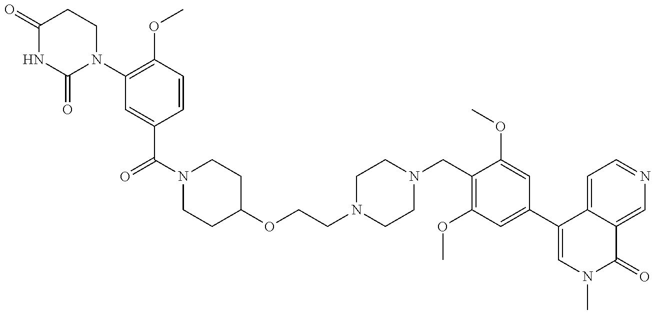 [SMILES: COc1ccc(C(=O)N2CCC(OCCN3CCN(Cc4c(OC)cc(-c5cn(C)c(=O)c6cnccc56)cc4OC)CC3)CC2)cc1N1CCC(=O)NC1=O] |
| A35 | 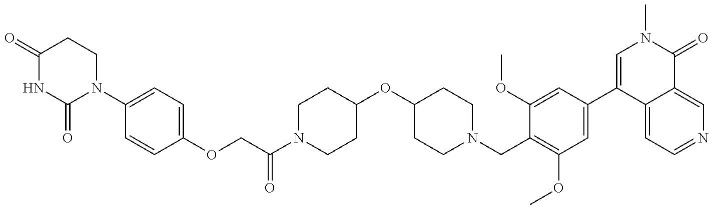 [SMILES: COc1cc(-c2cn(C)c(=O)c3cnccc23)cc(OC)c1CN1CCC(OC2CCN(C(=O)COc3ccc(N4CCC(=O)NC4=O)cc3)CC2)CC1] |
| A36 | 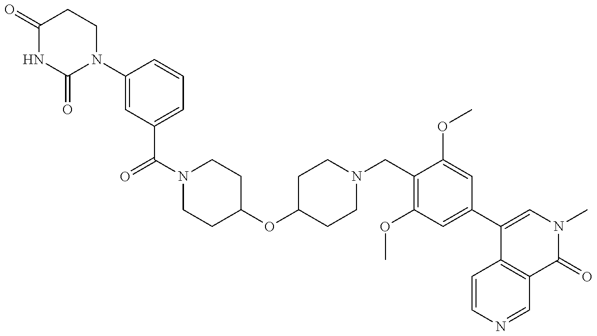 [SMILES: COc1cc(-c2cn(C)c(=O)c3cnccc23)cc(OC)c1CN1CCC(OC2CCN(C(=O)c3cccc(N4CCC(=O)NC4=O)c3)CC2)CC1] |
| A37 | 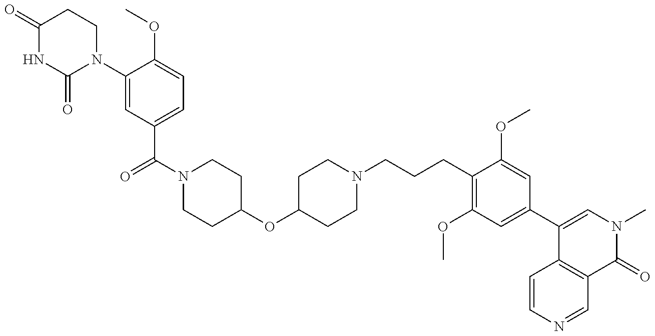 [SMILES: COc1ccc(C(=O)N2CCC(OC3CCN(CCCc4c(OC)cc(-c5cn(C)c(=O)c6cnccc56)cc4OC)CC3)CC2)cc1N1CCC(=O)NC1=O] |
| A38 | 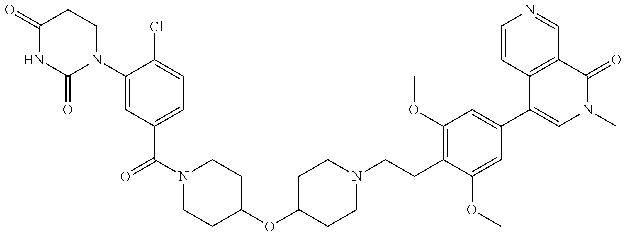 [SMILES: COc1cc(-c2cn(C)c(=O)c3cnccc23)cc(OC)c1CCN1CCC(OC2CCN(C(=O)c3ccc(Cl)c(N4CCC(=O)NC4=O)c3)CC2)CC1] |
| A39 | 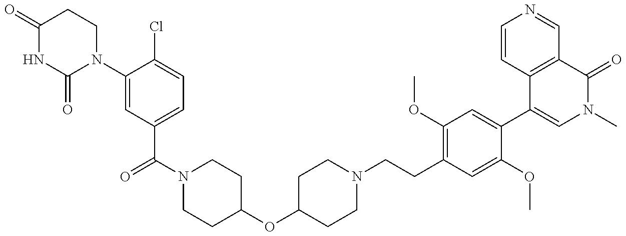 [SMILES: COc1cc(-c2cn(C)c(=O)c3cnccc23)c(OC)cc1CCN1CCC(OC2CCN(C(=O)c3ccc(Cl)c(N4CCC(=O)NC4=O)c3)CC2)CC1] |
| A40 | 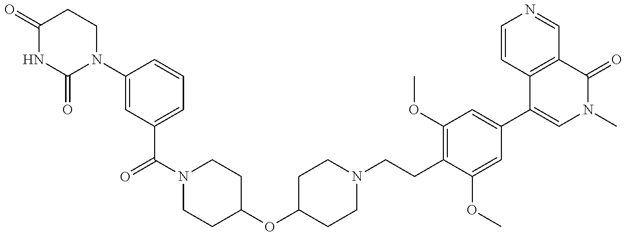 [SMILES: COc1cc(-c2cn(C)c(=O)c3cnccc23)cc(OC)c1CCN1CCC(OC2CCN(C(=O)c3cccc(N4CCC(=O)NC4=O)c3)CC2)CC1] |
| A41 | 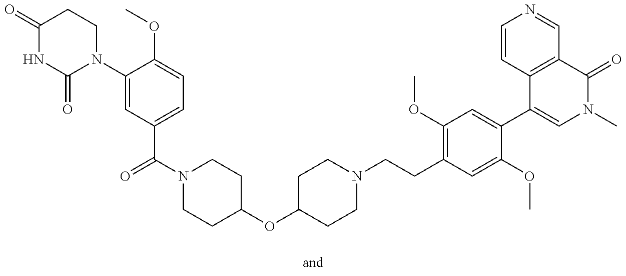 [SMILES: COc1cc(-c2cn(C)c(=O)c3cnccc23)c(OC)cc1CCN1CCC(OC2CCN(C(=O)c3ccc(OC)c(N4CCC(=O)NC4=O)c3)CC2)CC1] |
| A42 | 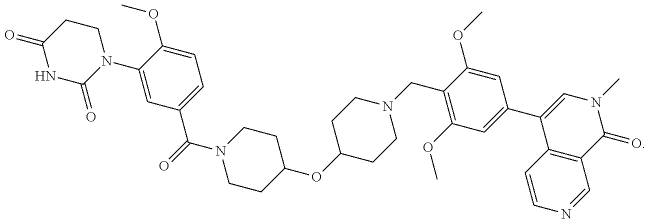 [SMILES: COc1ccc(C(=O)N2CCC(OC3CCN(Cc4c(OC)cc(-c5cn(C)c(=O)c6cnccc56)cc4OC)CC3)CC2)cc1N1CCC(=O)NC1=O] |

In an embodiment, the compound of a Formula above or a pharmaceutically acceptable salt, hydrate, solvate, prodrug, stereoisomer, or tautomer thereof, is selected from the group consisting of:

| Compound | |
|---|---|
| Number | Compound Structure |
| B1 | 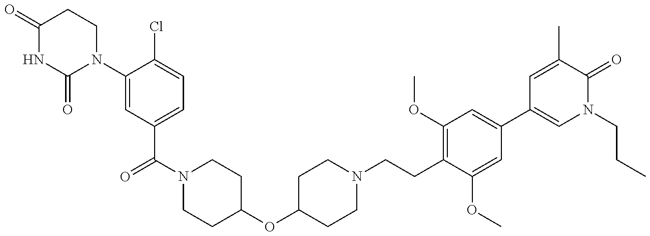 [SMILES: CCCn1cc(-c2cc(OC)c(CCN3CCC(OC4CCN(C(=O)c5ccc(Cl)c(N6CCC(=O)NC6=O)c5)CC4)CC3)c(OC)c2)cc(C)c1=O] |
| B2 | 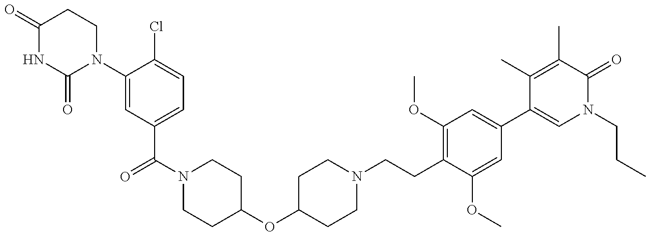 [SMILES: CCCn1cc(-c2cc(OC)c(CCN3CCC(OC4CCN(C(=O)c5ccc(Cl)c(N6CCC(=O)NC6=O)c5)CC4)CC3)c(OC)c2)c(C)c(C)c1=O] |
| B3 | 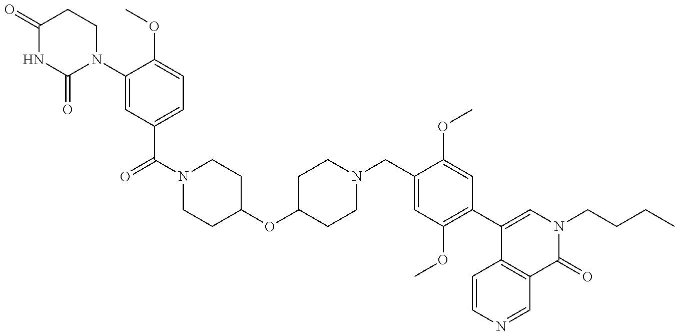 [SMILES: CCCCn1cc(-c2cc(OC)c(CN3CCC(OC4CCN(C(=O)c5ccc(OC)c(N6CCC(=O)NC6=O)c5)CC4)CC3)cc2OC)c2ccncc2c1=O] |
| B4 | 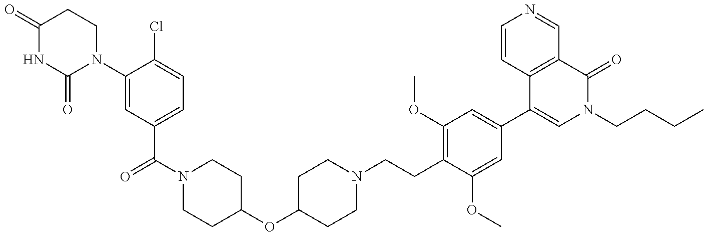 [SMILES: CCCCn1cc(-c2cc(OC)c(CCN3CCC(OC4CCN(C(=O)c5ccc(Cl)c(N6CCC(=O)NC6=O)c5)CC4)CC3)c(OC)c2)c2ccncc2c1=O] |
| B5 | 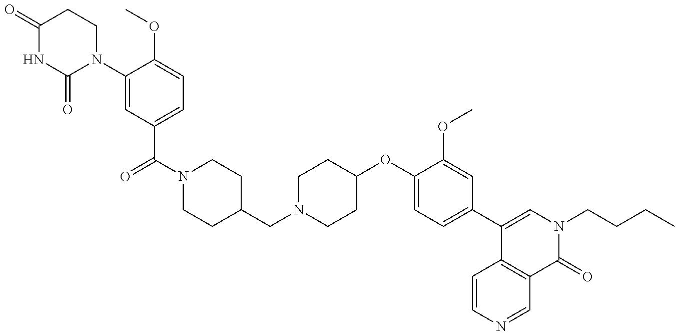 [SMILES: CCCCn1cc(-c2ccc(OC3CCN(CC4CCN(C(=O)c5ccc(OC)c(N6CCC(=O)NC6=O)c5)CC4)CC3)c(OC)c2)c2ccncc2c1=O] |
| B6 | 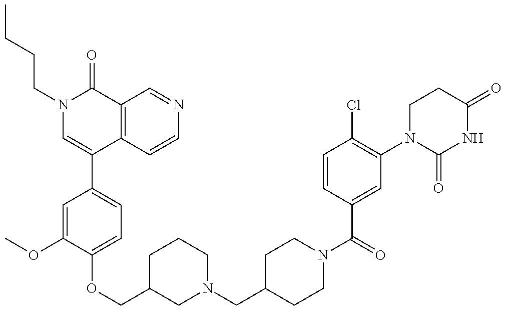 [SMILES: CCCCn1cc(-c2ccc(OCC3CCCN(CC4CCN(C(=O)c5ccc(Cl)c(N6CCC(=O)NC6=O)c5)CC4)C3)c(OC)c2)c2ccncc2c1=O] |
| B7 | 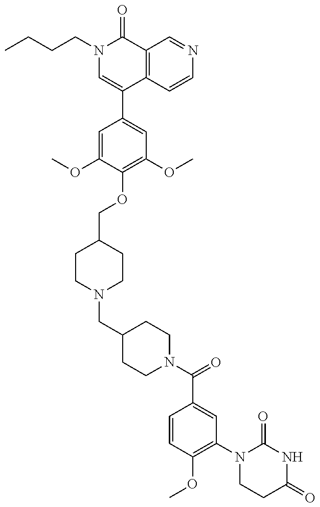 [SMILES: CCCCn1cc(-c2cc(OC)c(OCC3CCN(CC4CCN(C(=O)c5ccc(OC)c(N6CCC(=O)NC6=O)c5)CC4)CC3)c(OC)c2)c2ccncc2c1=O] |
| B8 | 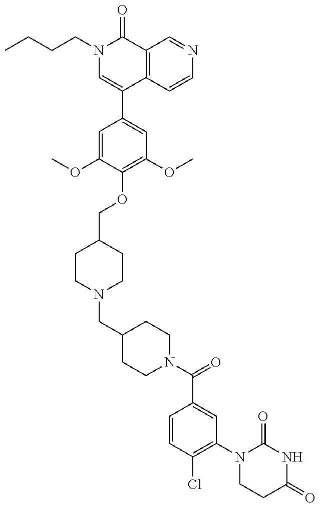 [SMILES: CCCCn1cc(-c2cc(OC)c(OCC3CCN(CC4CCN(C(=O)c5ccc(Cl)c(N6CCC(=O)NC6=O)c5)CC4)CC3)c(OC)c2)c2ccncc2c1=O] |
| B9 | 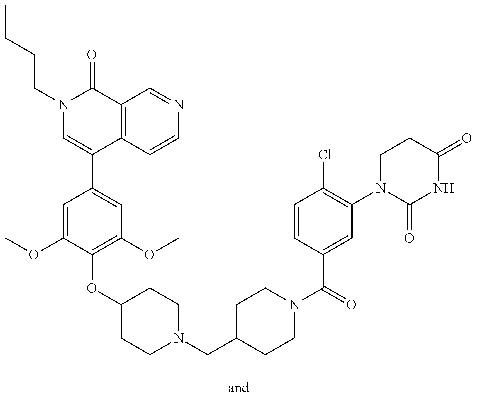 [SMILES: CCCCn1cc(-c2cc(OC)c(OC3CCN(CC4CCN(C(=O)c5ccc(Cl)c(N6CCC(=O)NC6=O)c5)CC4)CC3)c(OC)c2)c2ccncc2c1=O] |
| B10 | 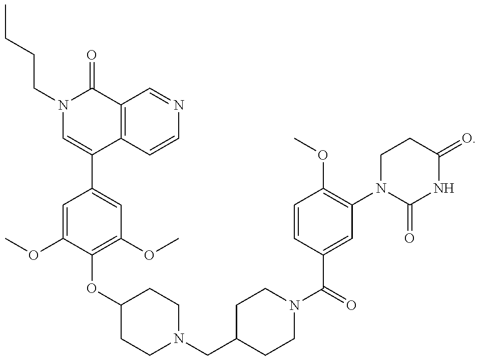 [SMILES: CCCCn1cc(-c2cc(OC)c(OC3CCN(CC4CCN(C(=O)c5ccc(OC)c(N6CCC(=O)NC6=O)c5)CC4)CC3)c(OC)c2)c2ccncc2c1=O] |

In an embodiment, the compound of a Formula above or a pharmaceutically acceptable salt, hydrate, solvate, prodrug, stereoisomer, or tautomer thereof, is selected from the group consisting of:

| Compound | |
|---|---|
| Number | Compound Structure |
| A1 | 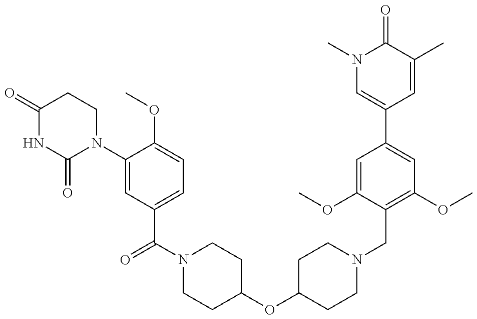 [SMILES: COc1ccc(C(=O)N2CCC(OC3CCN(Cc4c(OC)cc(-c5cc(C)c(=O)n(C)c5)cc4OC)CC3)CC2)cc1N1CCC(=O)NC1=O] |
| A2 | 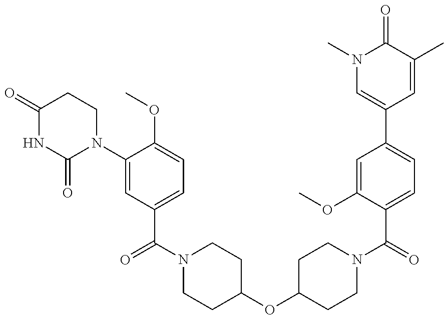 [SMILES: COc1cc(-c2cc(C)c(=O)n(C)c2)ccc1C(=O)N1CCC(OC2CCN(C(=O)c3ccc(OC)c(N4CCC(=O)NC4=O)c3)CC2)CC1] |
| A3 | 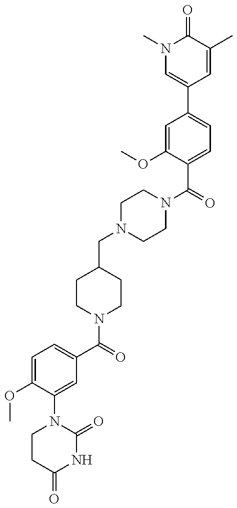 [SMILES: COc1cc(-c2cc(C)c(=O)n(C)c2)ccc1C(=O)N1CCN(CC2CCN(C(=O)c3ccc(OC)c(N4CCC(=O)NC4=O)c3)CC2)CC1] |
| A4 | 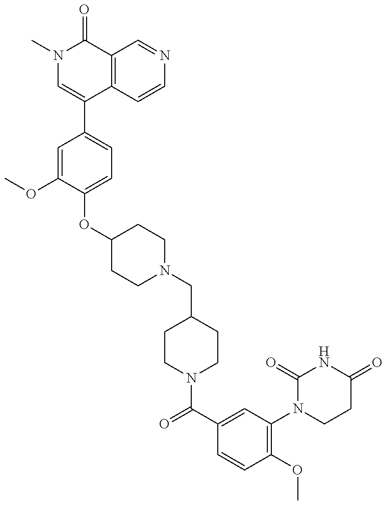 [SMILES: COc1cc(-c2cn(C)c(=O)c3cnccc23)ccc1OC1CCN(CC2CCN(C(=O)c3ccc(OC)c(N4CCC(=O)NC4=O)c3)CC2)CC1] |
| A5 | 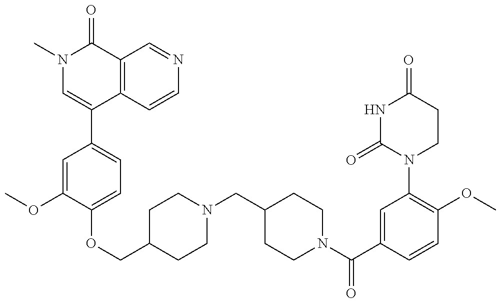 |
| A6 | 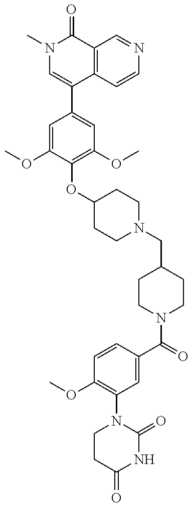 [SMILES: COc1ccc(C(=O)N2CCC(CN3CCC(Oc4c(OC)cc(-c5cn(C)c(=O)c6cnccc56)cc4OC)CC3)CC2)cc1N1CCC(=O)NC1=O] |
| A7 | 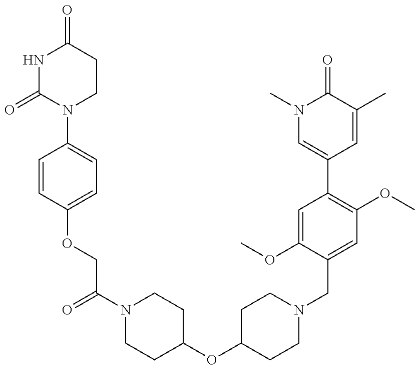 [SMILES: COc1cc(-c2cc(C)c(=O)n(C)c2)c(OC)cc1CN1CCC(OC2CCN(C(=O)COc3ccc(N4CCC(=O)NC4=O)cc3)CC2)CC1] |
| A8 | 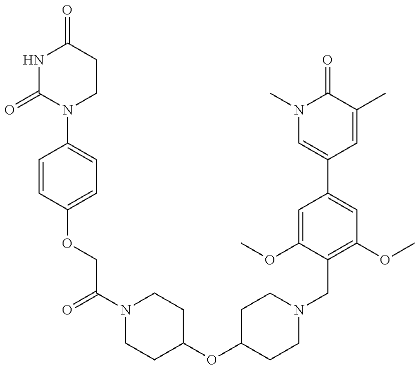 [SMILES: COc1cc(-c2cc(C)c(=O)n(C)c2)cc(OC)c1CN1CCC(OC2CCN(C(=O)COc3ccc(N4CCC(=O)NC4=O)cc3)CC2)CC1] |
| A9 | 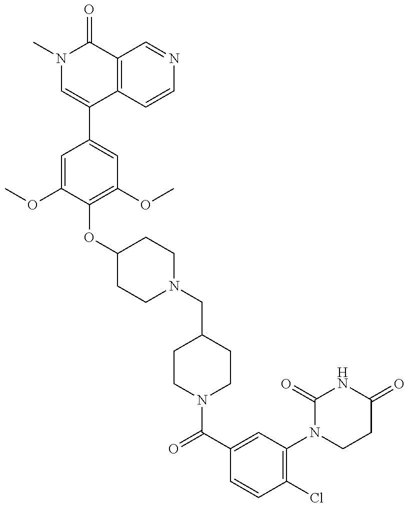 [SMILES: COc1cc(-c2cn(C)c(=O)c3cnccc23)cc(OC)c1OC1CCN(CC2CCN(C(=O)c3ccc(Cl)c(N4CCC(=O)NC4=O)c3)CC2)CC1] |
| A10 | 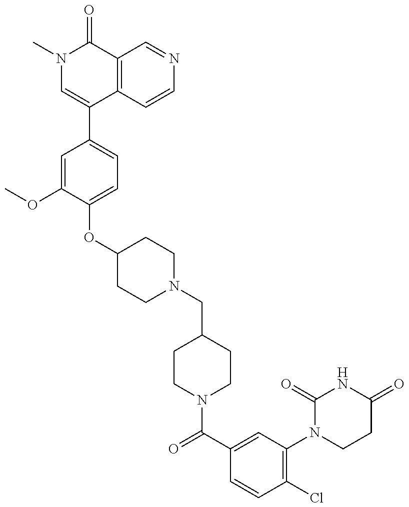 [SMILES: COc1cc(-c2cn(C)c(=O)c3cnccc23)ccc1OC1CCN(CC2CCN(C(=O)c3ccc(Cl)c(N4CCC(=O)NC4=O)c3)CC2)CC1] |
| A11 | 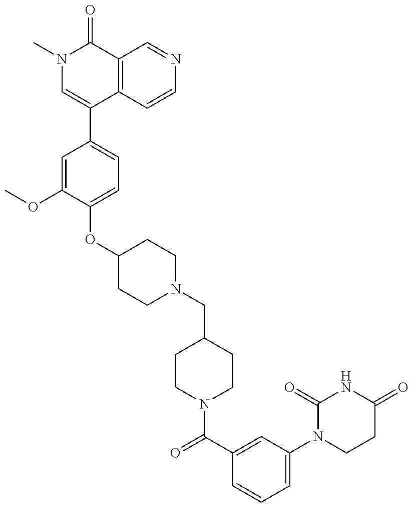 [SMILES: COc1cc(-c2cn(C)c(=O)c3cnccc23)ccc1OC1CCN(CC2CCN(C(=O)c3cccc(N4CCC(=O)NC4=O)c3)CC2)CC1] |
| A12 | 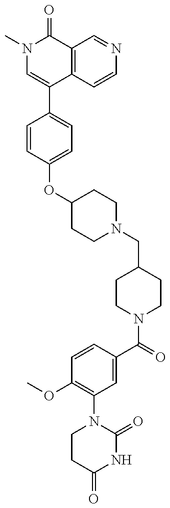 [SMILES: COc1ccc(C(=O)N2CCC(CN3CCC(Oc4ccc(-c5cn(C)c(=O)c6cnccc56)cc4)CC3)CC2)cc1N1CCC(=O)NC1=O] |
| A13 | 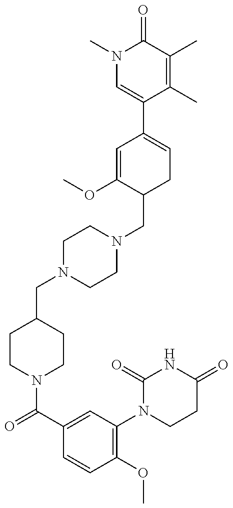 [SMILES: COC1=CC(c2cn(C)c(=O)c(C)c2C)=CCC1CN1CCN(CC2CCN(C(=O)c3ccc(OC)c(N4CCC(=O)NC4=O)c3)CC2)CC1] |
| A14 | 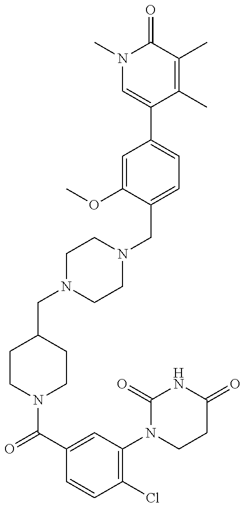 [SMILES: COc1cc(-c2cn(C)c(=O)c(C)c2C)ccc1CN1CCN(CC2CCN(C(=O)c3ccc(Cl)c(N4CCC(=O)NC4=O)c3)CC2)CC1] |
| A15 | 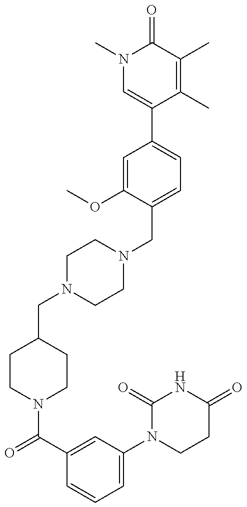 [SMILES: COc1cc(-c2cn(C)c(=O)c(C)c2C)ccc1CN1CCN(CC2CCN(C(=O)c3cccc(N4CCC(=O)NC4=O)c3)CC2)CC1] |
| A16 | 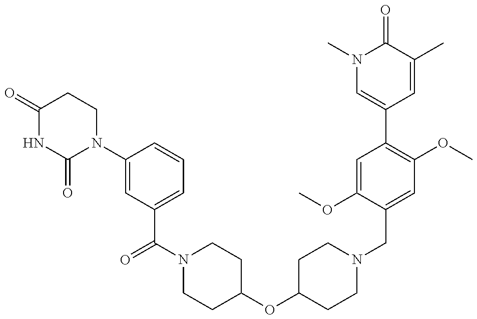 [SMILES: COc1cc(-c2cc(C)c(=O)n(C)c2)c(OC)cc1CN1CCC(OC2CCN(C(=O)c3cccc(N4CCC(=O)NC4=O)c3)CC2)CC1] |
| A17 | 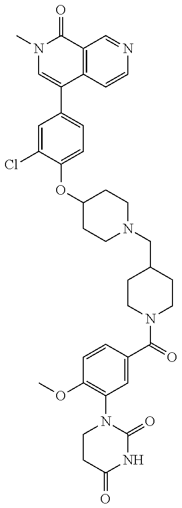 [SMILES: COc1ccc(C(=O)N2CCC(CN3CCC(Oc4ccc(-c5cn(C)c(=O)c6cnccc56)cc4Cl)CC3)CC2)cc1N1CCC(=O)NC1=O] |
| A18 | 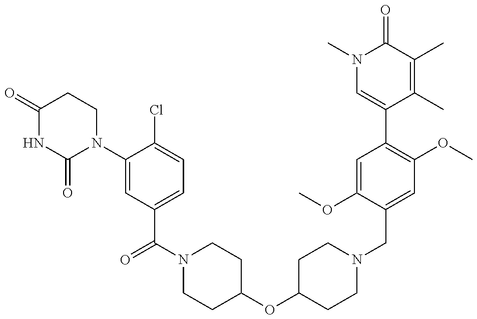 [SMILES: COc1cc(-c2cn(C)c(=O)c(C)c2C)c(OC)cc1CN1CCC(OC2CCN(C(=O)c3ccc(Cl)c(N4CCC(=O)NC4=O)c3)CC2)CC1] |
| A19 | 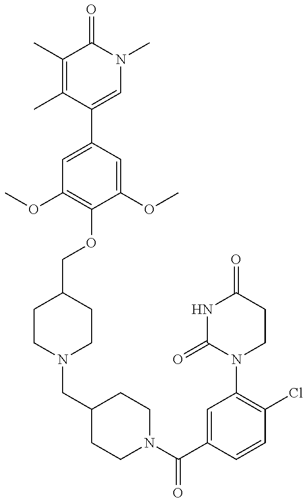 [SMILES: COc1cc(-c2cn(C)c(=O)c(C)c2C)cc(OC)c1OCC1CCN(CC2CCN(C(=O)c3ccc(Cl)c(N4CCC(=O)NC4=O)c3)CC2)CC1] |
| A20 | 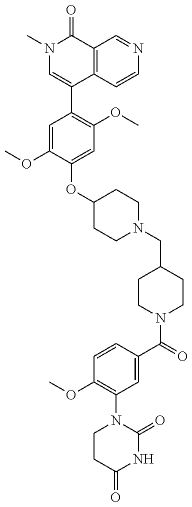 [SMILES: COc1cc(-c2cn(C)c(=O)c3cnccc23)c(OC)cc1OC1CCN(CC2CCN(C(=O)c3ccc(OC)c(N4CCC(=O)NC4=O)c3)CC2)CC1] |
| A21 | 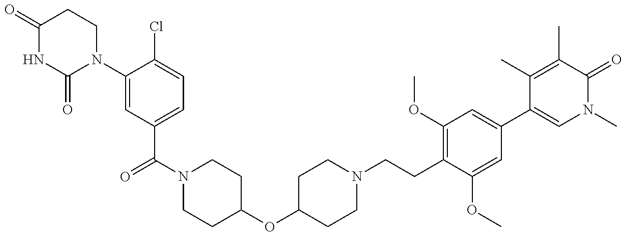 [SMILES: COc1cc(-c2cn(C)c(=O)c(C)c2C)cc(OC)c1CCN1CCC(OC2CCN(C(=O)c3ccc(Cl)c(N4CCC(=O)NC4=O)c3)CC2)CC1] |
| A22 | 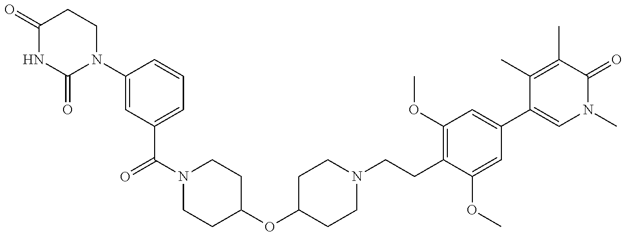 [SMILES: COc1cc(-c2cn(C)c(=O)c(C)c2C)cc(OC)c1CCN1CCC(OC2CCN(C(=O)c3cccc(N4CCC(=O)NC4=O)c3)CC2)CC1] |
| A23 | 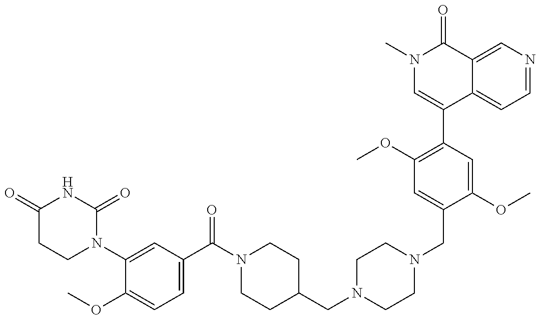 [SMILES: COc1cc(-c2cn(C)c(=O)c3cnccc23)c(OC)cc1CN1CCN(CC2CCN(C(=O)c3ccc(OC)c(N4CCC(=O)NC4=O)c3)CC2)CC1] |
| A24 | 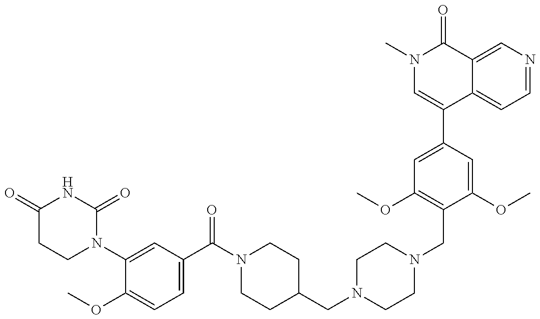 [SMILES: COc1ccc(C(=O)N2CCC(CN3CCN(Cc4c(OC)cc(-c5cn(C)c(=O)c6cnccc56)cc4OC)CC3)CC2)cc1N1CCC(=O)NC1=O] |
| A25 | 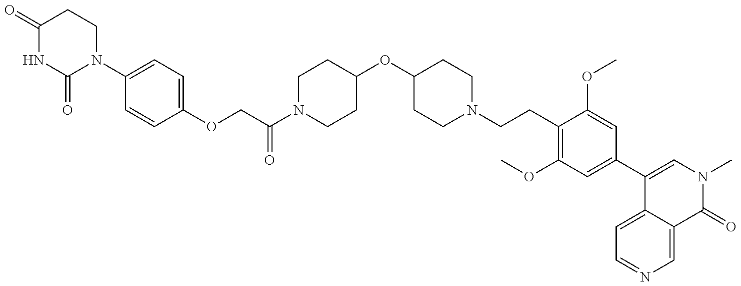 [SMILES: COc1cc(-c2cn(C)c(=O)c3cnccc23)cc(OC)c1CCN1CCC(OC2CCN(C(=O)COc3ccc(N4CCC(=O)NC4=O)cc3)CC2)CC1] |
| A26 | 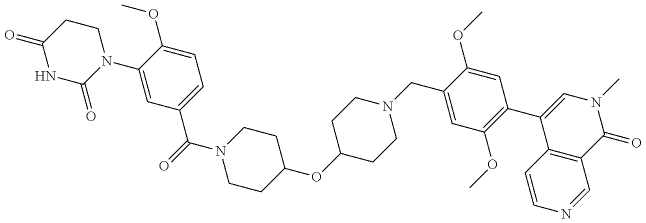 [SMILES: COc1cc(-c2cn(C)c(=O)c3cnccc23)c(OC)cc1CN1CCC(OC2CCN(C(=O)c3ccc(OC)c(N4CCC(=O)NC4=O)c3)CC2)CC1] |
| A27 | 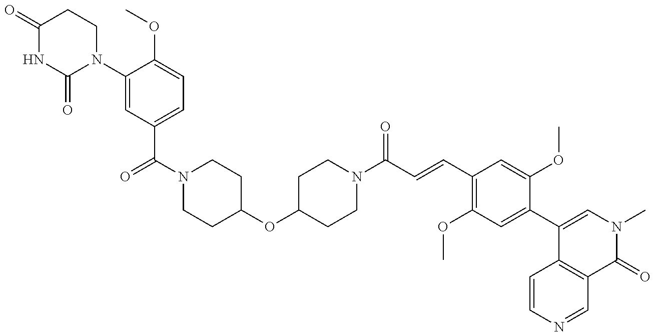 [SMILES: COc1cc(-c2cn(C)c(=O)c3cnccc23)c(OC)cc1/C=C/C(=O)N1CCC(OC2CCN(C(=O)c3ccc(OC)c(N4CCC(=O)NC4=O)c3)CC2)CC1] |
| A28 | 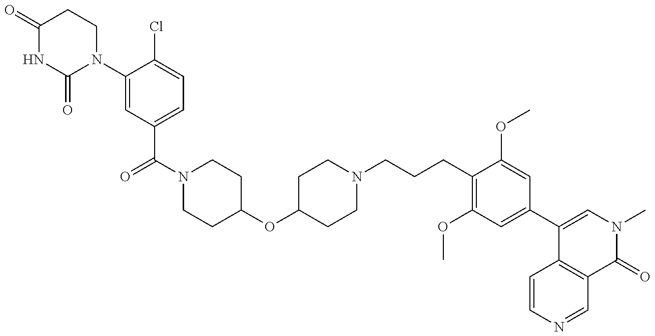 [SMILES: COc1cc(-c2cn(C)c(=O)c3cnccc23)cc(OC)c1CCCN1CCC(OC2CCN(C(=O)c3ccc(Cl)c(N4CCC(=O)NC4=O)c3)CC2)CC1] |
| A29 | 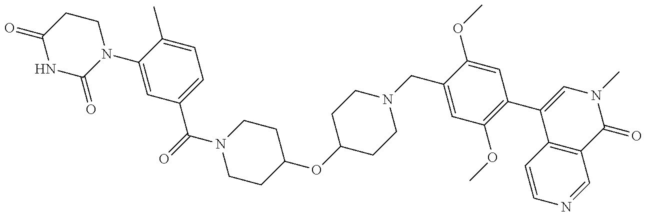 [SMILES: COc1cc(-c2cn(C)c(=O)c3cnccc23)c(OC)cc1CN1CCC(OC2CCN(C(=O)c3ccc(C)c(N4CCC(=O)NC4=O)c3)CC2)CC1] |
| A30 | 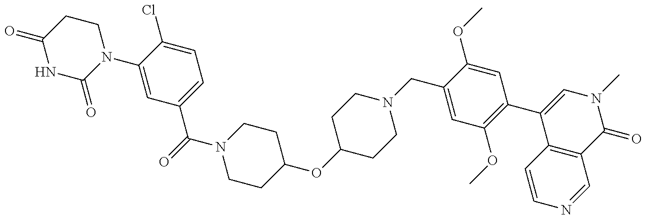 [SMILES: COc1cc(-c2cn(C)c(=O)c3cnccc23)c(OC)cc1CN1CCC(OC2CCN(C(=O)c3ccc(Cl)c(N4CCC(=O)NC4=O)c3)CC2)CC1] |
| A31 | 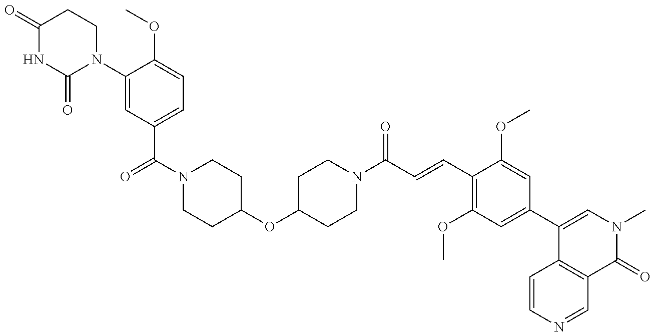 [SMILES: COc1ccc(C(=O)N2CCC(OC3CCN(C(=O)/C=C/c4c(OC)cc(-c5cn(C)c(=O)c6cnccc56)cc4OC)CC3)CC2)cc1N1CCC(=O)NC1=O] |
| A32 | 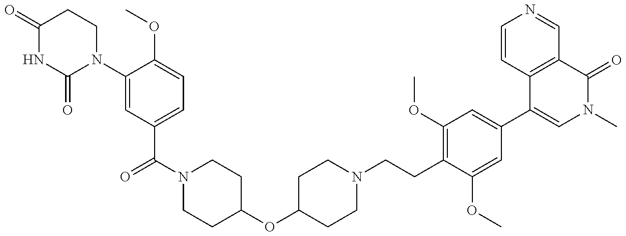 [SMILES: COc1ccc(C(=O)N2CCC(OC3CCN(CCc4c(OC)cc(-c5cn(C)c(=O)c6cnccc56)cc4OC)CC3)CC2)cc1N1CCC(=O)NC1=O] |
| A33 | 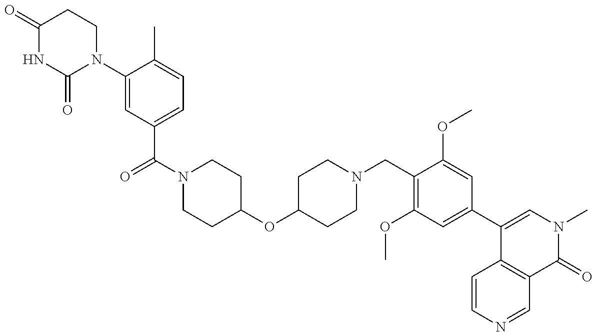 [SMILES: COc1cc(-c2cn(C)c(=O)c3cnccc23)cc(OC)c1CN1CCC(OC2CCN(C(=O)c3ccc(C)c(N4CCC(=O)NC4=O)c3)CC2)CC1] |
| A34 | 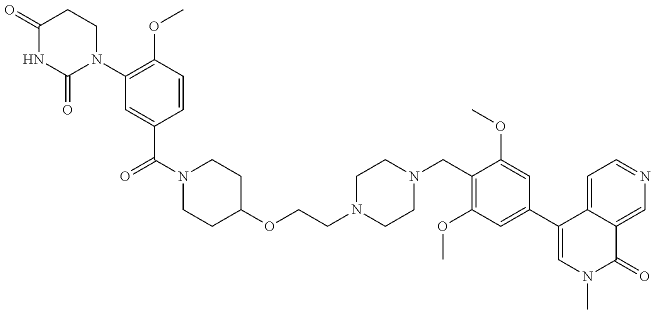 [SMILES: COc1ccc(C(=O)N2CCC(OCCN3CCN(Cc4c(OC)cc(-c5cn(C)c(=O)c6cnccc56)cc4OC)CC3)CC2)cc1N1CCC(=O)NC1=O] |
| A35 | 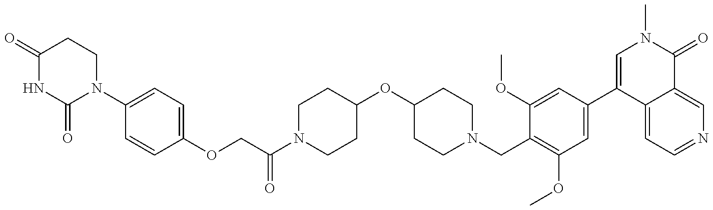 [SMILES: COc1cc(-c2cn(C)c(=O)c3cnccc23)cc(OC)c1CN1CCC(OC2CCN(C(=O)COc3ccc(N4CCC(=O)NC4=O)cc3)CC2)CC1] |
| A36 | 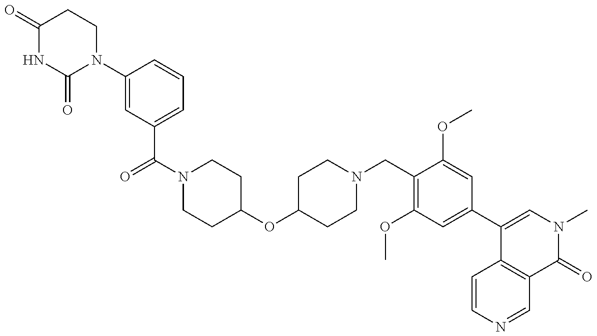 [SMILES: COc1cc(-c2cn(C)c(=O)c3cnccc23)cc(OC)c1CN1CCC(OC2CCN(C(=O)c3cccc(N4CCC(=O)NC4=O)c3)CC2)CC1] |
| A37 | 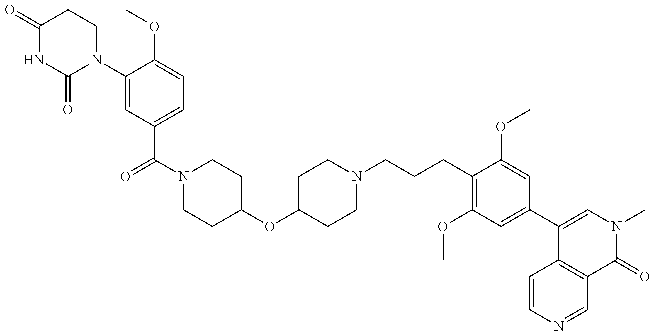 [SMILES: COc1ccc(C(=O)N2CCC(OC3CCN(CCCc4c(OC)cc(-c5cn(C)c(=O)c6cnccc56)cc4OC)CC3)CC2)cc1N1CCC(=O)NC1=O] |
| A38 | 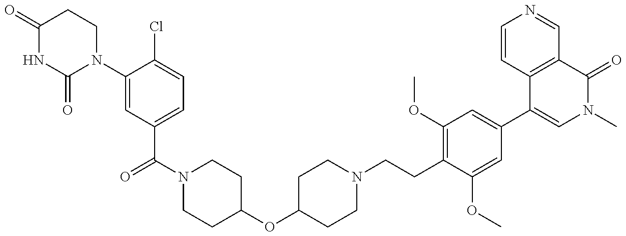 [SMILES: COc1cc(-c2cn(C)c(=O)c3cnccc23)cc(OC)c1CCN1CCC(OC2CCN(C(=O)c3ccc(Cl)c(N4CCC(=O)NC4=O)c3)CC2)CC1] |
| A39 | 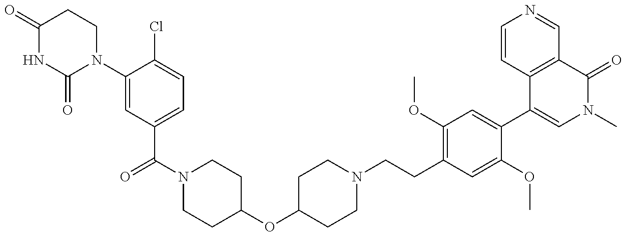 [SMILES: COc1cc(-c2cn(C)c(=O)c3cnccc23)c(OC)cc1CCN1CCC(OC2CCN(C(=O)c3ccc(Cl)c(N4CCC(=O)NC4=O)c3)CC2)CC1] |
| A40 | 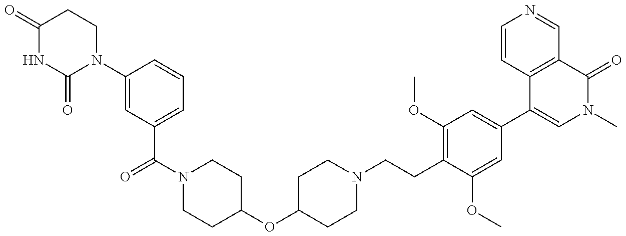 [SMILES: COc1cc(-c2cn(C)c(=O)c3cnccc23)cc(OC)c1CCN1CCC(OC2CCN(C(=O)c3cccc(N4CCC(=O)NC4=O)c3)CC2)CC1] |
| A41 | 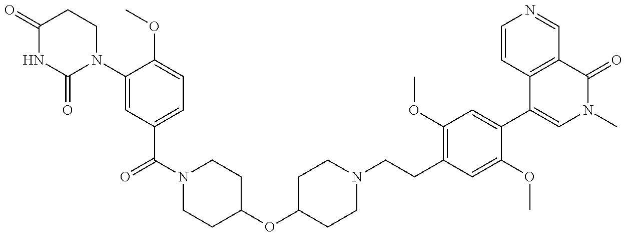 [SMILES: COc1cc(-c2cn(C)c(=O)c3cnccc23)c(OC)cc1CCN1CCC(OC2CCN(C(=O)c3ccc(OC)c(N4CCC(=O)NC4=O)c3)CC2)CC1] |
| A42 | 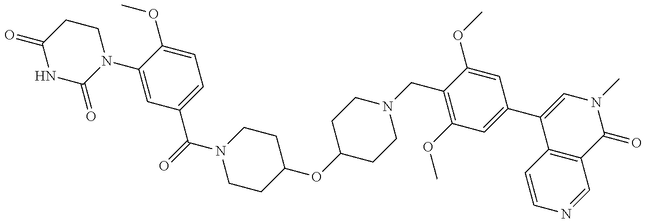 [SMILES: COc1ccc(C(=O)N2CCC(OC3CCN(Cc4c(OC)cc(-c5cn(C)c(=O)c6cnccc56)cc4OC)CC3)CC2)cc1N1CCC(=O)NC1=O] |
| B1 | 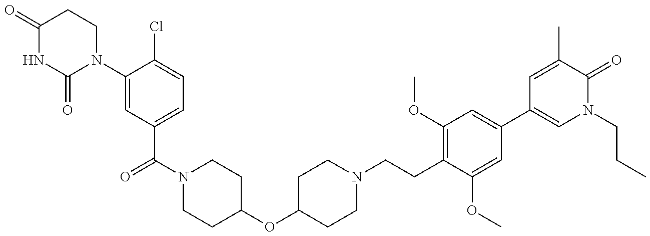 [SMILES: CCCn1cc(-c2cc(OC)c(CCN3CCC(OC4CCN(C(=O)c5ccc(Cl)c(N6CCC(=O)NC6=O)c5)CC4)CC3)c(OC)c2)cc(C)c1=O] |
| B2 | 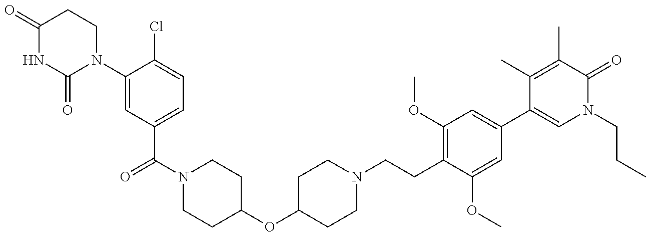 [SMILES: CCCn1cc(-c2cc(OC)c(CCN3CCC(OC4CCN(C(=O)c5ccc(Cl)c(N6CCC(=O)NC6=O)c5)CC4)CC3)c(OC)c2)c(C)c(C)c1=O] |
| B3 | 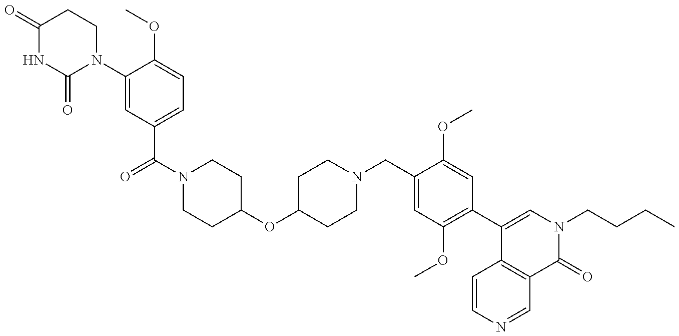 [SMILES: CCCCn1cc(-c2cc(OC)c(CN3CCC(OC4CCN(C(=O)c5ccc(OC)c(N6CCC(=O)NC6=O)c5)CC4)CC3)cc2OC)c2ccncc2c1=O] |
| B4 | 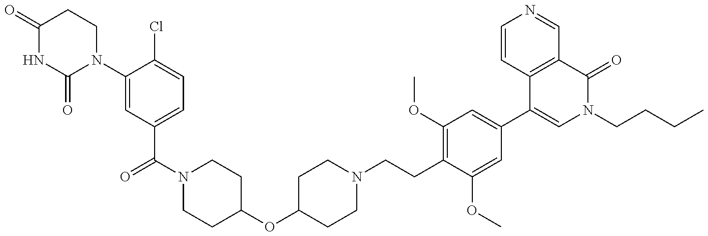 [SMILES: CCCCn1cc(-c2cc(OC)c(CCN3CCC(OC4CCN(C(=O)c5ccc(Cl)c(N6CCC(=O)NC6=O)c5)CC4)CC3)c(OC)c2)c2ccncc2c1=O] |
| B5 | 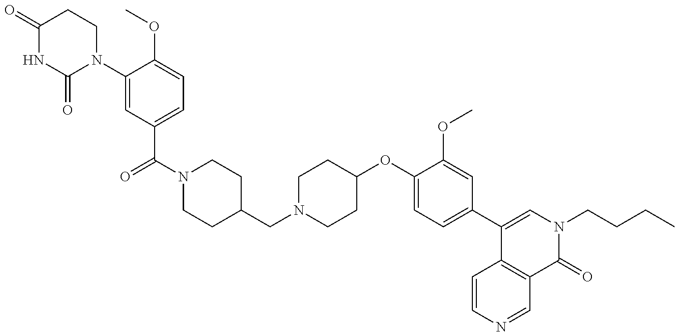 [SMILES: CCCCn1cc(-c2ccc(OC3CCN(CC4CCN(C(=O)c5ccc(OC)c(N6CCC(=O)NC6=O)c5)CC4)CC3)c(OC)c2)c2ccncc2c1=O] |
| B6 | 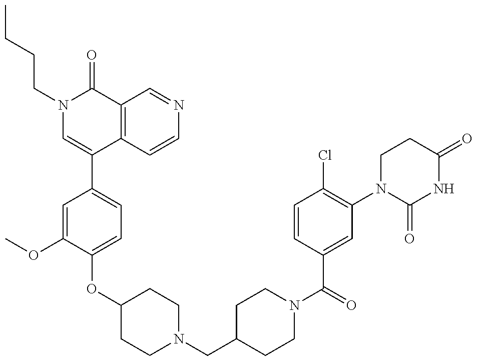 [SMILES: CCCCn1cc(-c2ccc(OC3CCN(CC4CCN(C(=O)c5ccc(Cl)c(N6CCC(=O)NC6=O)c5)CC4)CC3)c(OC)c2)c2ccncc2c1=O] |
| B7 | 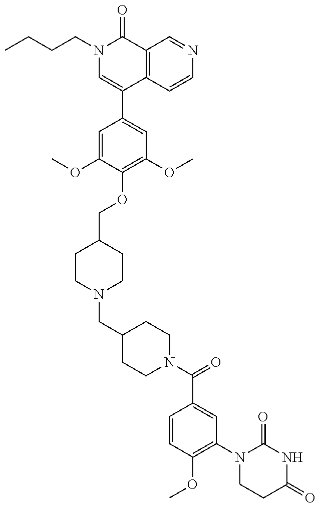 [SMILES: CCCCn1cc(-c2cc(OC)c(OCC3CCN(CC4CCN(C(=O)c5ccc(OC)c(N6CCC(=O)NC6=O)c5)CC4)CC3)c(OC)c2)c2ccncc2c1=O] |
| B8 | 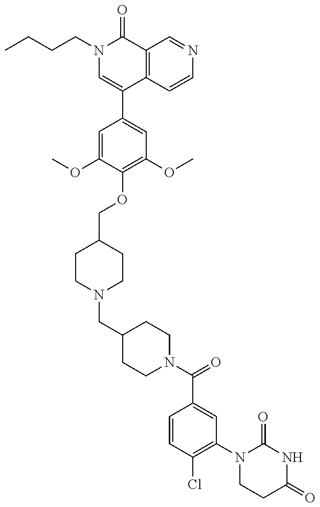 [SMILES: CCCCn1cc(-c2cc(OC)c(OCC3CCN(CC4CCN(C(=O)c5ccc(Cl)c(N6CCC(=O)NC6=O)c5)CC4)CC3)c(OC)c2)c2ccncc2c1=O] |
| B9 | 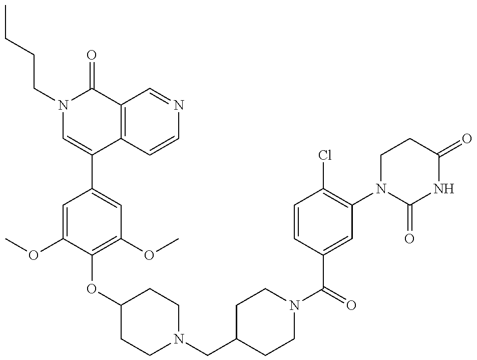 [SMILES: CCCCn1cc(-c2cc(OC)c(OC3CCN(CC4CCN(C(=O)c5ccc(Cl)c(N6CCC(=O)NC6=O)c5)CC4)CC3)c(OC)c2)c2ccncc2c1=O] |
| B10 | 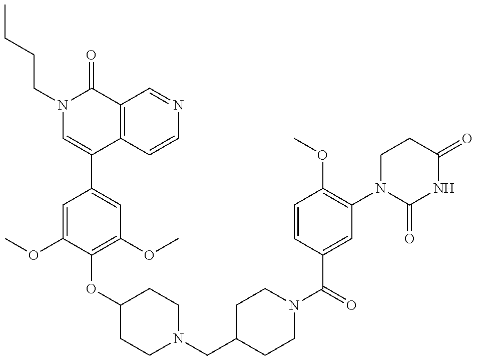 [SMILES: CCCCn1cc(-c2cc(OC)c(OC3CCN(CC4CCN(C(=O)c5ccc(OC)c(N6CCC(=O)NC6=O)c5)CC4)CC3)c(OC)c2)c2ccncc2c1=O] |
| E3 | 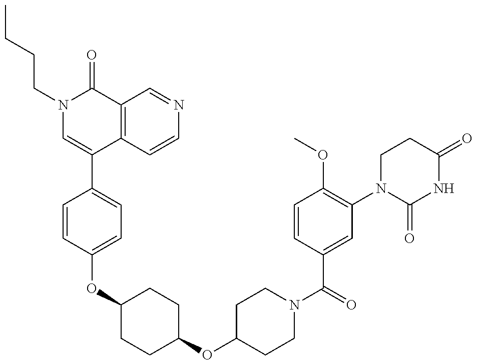 [SMILES: CCCCn1cc(-c2ccc(O[C@H]3CC[C@@H](OC4CCN(C(=O)c5ccc(OC)c(N6CCC(=O)NC6=O)c5)CC4)CC3)cc2)c2ccncc2c1=O] |
| E4 | 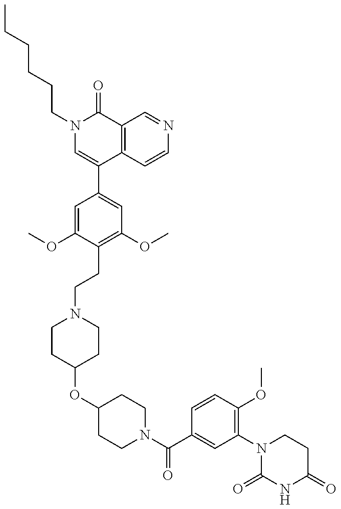 [SMILES: CCCCCCn1cc(-c2cc(OC)c(CCN3CCC(OC4CCN(C(=O)c5ccc(OC)c(N6CCC(=O)NC6=O)c5)CC4)CC3)c(OC)c2)c2ccncc2c1=O] |
| E5 | 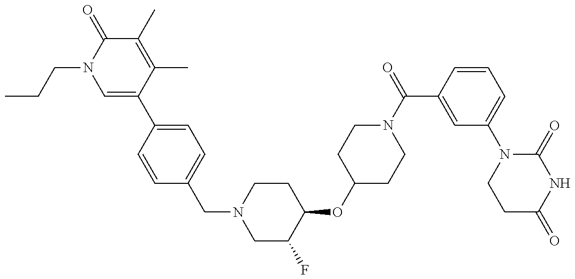 [SMILES: CCCn1cc(-c2ccc(CN3CC[C@@H](OC4CCN(C(=O)c5cccc(N6CCC(=O)NC6=O)c5)CC4)[C@H](F)C3)cc2)c(C)c(C)c1=O] |
| E6 | 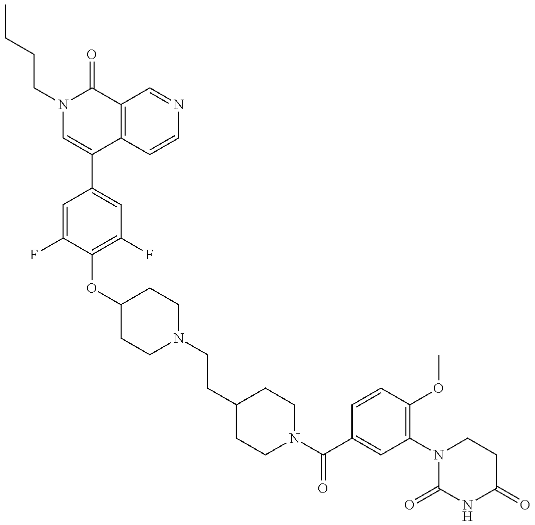 [SMILES: CCCCn1cc(-c2cc(F)c(OC3CCN(CCC4CCN(C(=O)c5ccc(OC)c(N6CCC(=O)NC6=O)c5)CC4)CC3)c(F)c2)c2ccncc2c1=O] |
| E12 | 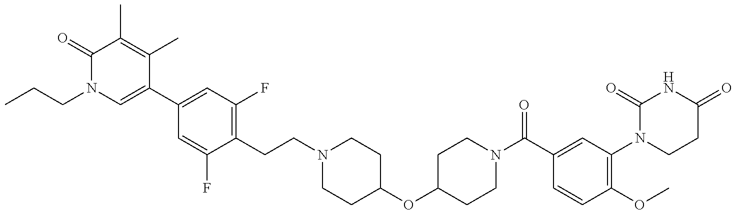 [SMILES: CCCn1cc(-c2cc(F)c(CCN3CCC(OC4CCN(C(=O)c5ccc(OC)c(N6CCC(=O)NC6=O)c5)CC4)CC3)c(F)c2)c(C)c(C)c1=O] |
| E13 | 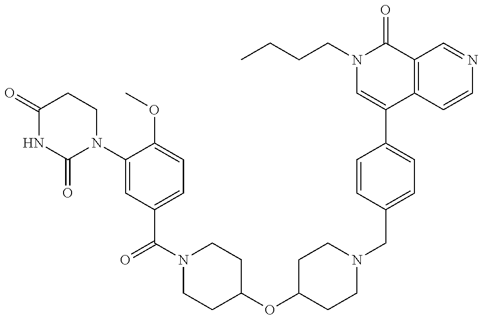 [SMILES: CCCCn1cc(-c2ccc(CN3CCC(OC4CCN(C(=O)c5ccc(OC)c(N6CCC(=O)NC6=O)c5)CC4)CC3)cc2)c2ccncc2c1=O] |
| E14 | 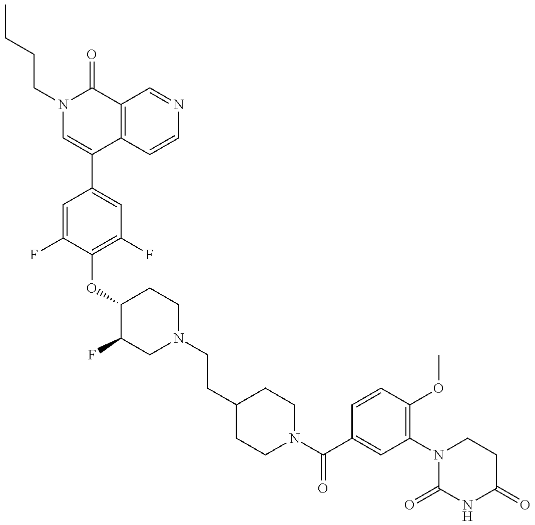 [SMILES: CCCCn1cc(-c2cc(F)c(O[C@@H]3CCN(CCC4CCN(C(=O)c5ccc(OC)c(N6CCC(=O)NC6=O)c5)CC4)C[C@H]3F)c(F)c2)c2ccncc2c1=O] |
| E15 | 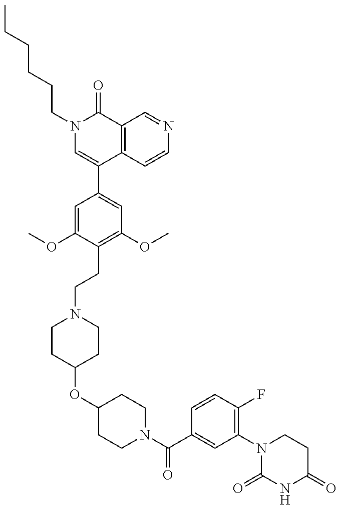 [SMILES: CCCCCCn1cc(-c2cc(OC)c(CCN3CCC(OC4CCN(C(=O)c5ccc(F)c(N6CCC(=O)NC6=O)c5)CC4)CC3)c(OC)c2)c2ccncc2c1=O] |
| E16 | 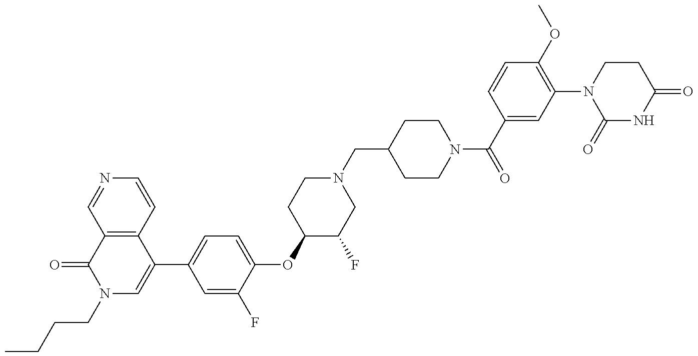 [SMILES: CCCCn1cc(-c2ccc(O[C@H]3CCN(CC4CCN(C(=O)c5ccc(OC)c(N6CCC(=O)NC6=O)c5)CC4)C[C@@H]3F)c(F)c2)c2ccncc2c1=O] |
| E22 | 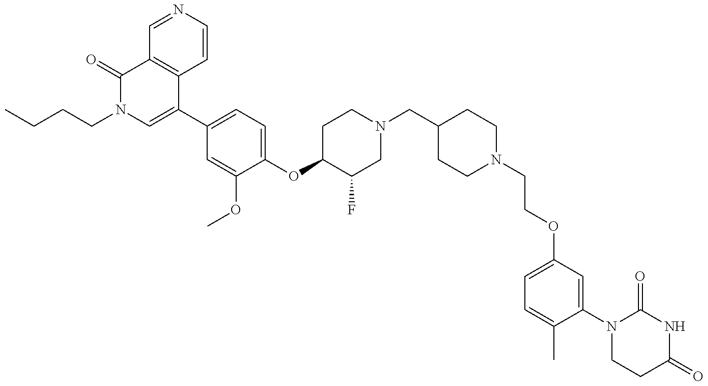 [SMILES: CCCCn1cc(-c2ccc(O[C@H]3CCN(CC4CCN(CCOc5ccc(C)c(N6CCC(=O)NC6=O)c5)CC4)C[C@@H]3F)c(OC)c2)c2ccncc2c1=O] |
| E25 | 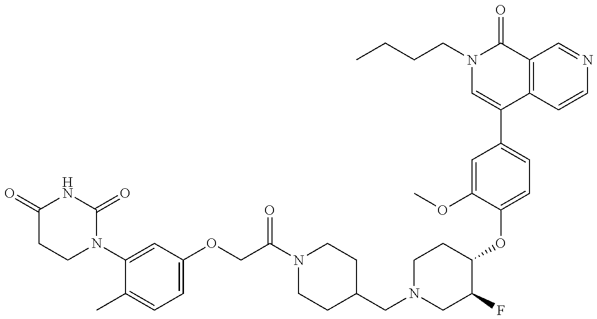 [SMILES: CCCCn1cc(-c2ccc(O[C@H]3CCN(CC4CCN(C(=O)COc5ccc(C)c(N6CCC(=O)NC6=O)c5)CC4)C[C@@H]3F)c(OC)c2)c2ccncc2c1=O] |
| E29 | 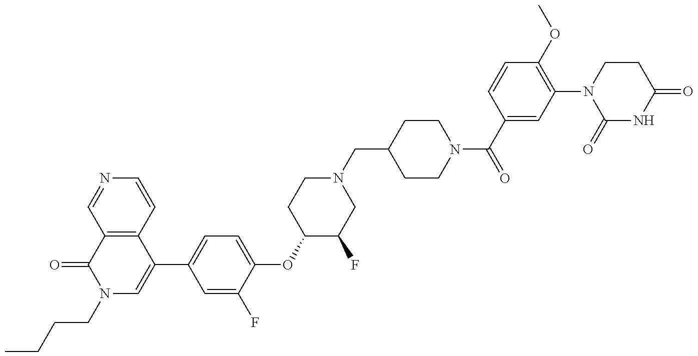 [SMILES: CCCCn1cc(-c2ccc(O[C@@H]3CCN(CC4CCN(C(=O)c5ccc(OC)c(N6CCC(=O)NC6=O)c5)CC4)C[C@H]3F)c(F)c2)c2ccncc2c1=O] |
| E31 | 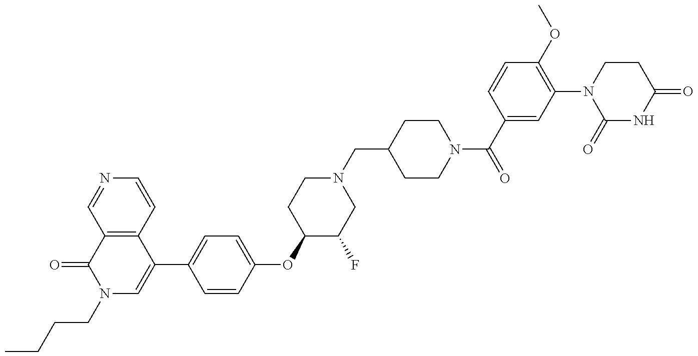 [SMILES: CCCCn1cc(-c2ccc(O[C@H]3CCN(CC4CCN(C(=O)c5ccc(OC)c(N6CCC(=O)NC6=O)c5)CC4)C[C@@H]3F)cc2)c2ccncc2c1=O] |
| E32 | 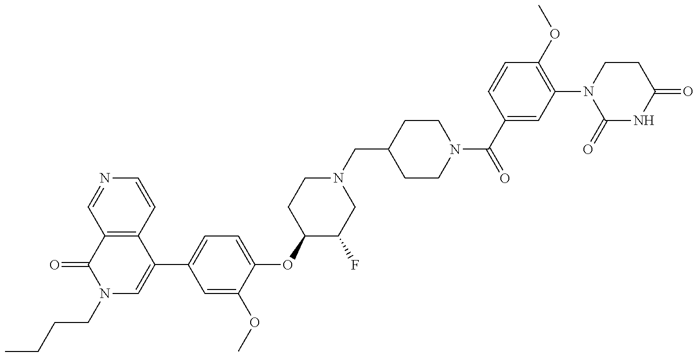 [SMILES: CCCCn1cc(-c2ccc(O[C@H]3CCN(CC4CCN(C(=O)c5ccc(OC)c(N6CCC(=O)NC6=O)c5)CC4)C[C@@H]3F)c(OC)c2)c2ccncc2c1=O] |
| E33 | 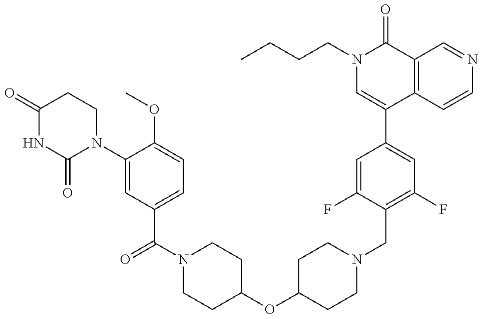 [SMILES: CCCCn1cc(-c2cc(F)c(CN3CCC(OC4CCN(C(=O)c5ccc(OC)c(N6CCC(=O)NC6=O)c5)CC4)CC3)c(F)c2)c2ccncc2c1=O] |
| E34 | 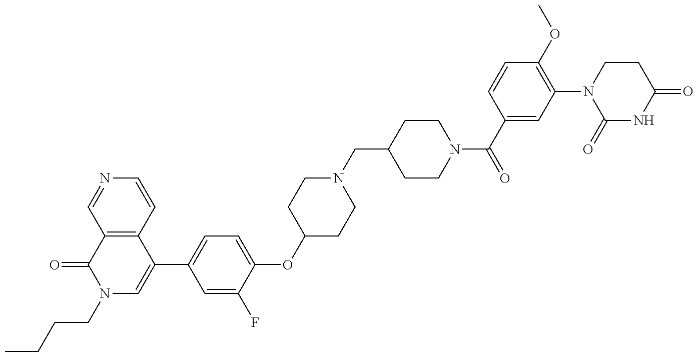 [SMILES: CCCCn1cc(-c2ccc(OC3CCN(CC4CCN(C(=O)c5ccc(OC)c(N6CCC(=O)NC6=O)c5)CC4)CC3)c(F)c2)c2ccncc2c1=O] |
| E36 | 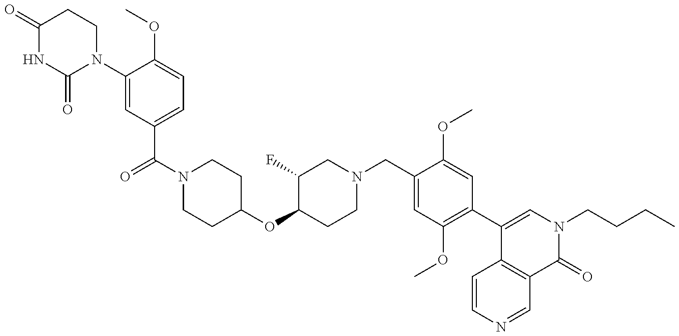 [SMILES: CCCCn1cc(-c2cc(OC)c(CN3CC[C@@H](OC4CCN(C(=O)c5ccc(OC)c(N6CCC(=O)NC6=O)c5)CC4)[C@H](F)C3)cc2OC)c2ccncc2c1=O] |
| E40 | 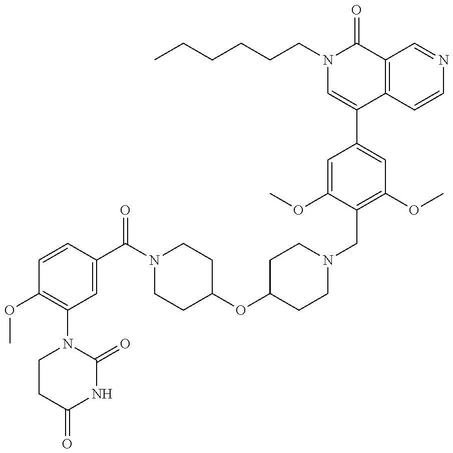 [SMILES: CCCCCCn1cc(-c2cc(OC)c(CN3CCC(OC4CCN(C(=O)c5ccc(OC)c(N6CCC(=O)NC6=O)c5)CC4)CC3)c(OC)c2)c2ccncc2c1=O] |
| E42 | 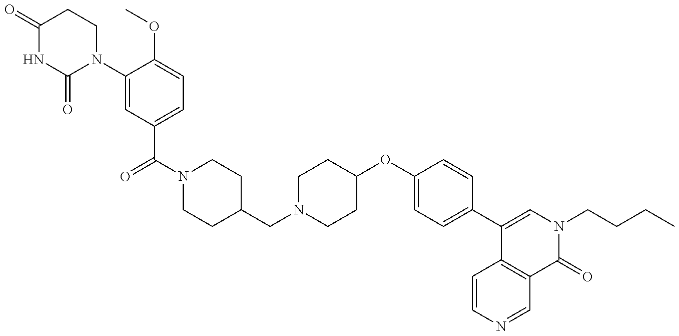 [SMILES: CCCCn1cc(-c2ccc(OC3CCN(CC4CCN(C(=O)c5ccc(OC)c(N6CCC(=O)NC6=O)c5)CC4)CC3)cc2)c2ccncc2c1=O] |
| E43 | 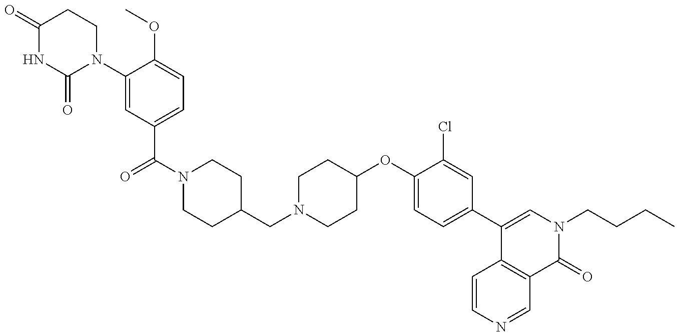 [SMILES: CCCCn1cc(-c2ccc(OC3CCN(CC4CCN(C(=O)c5ccc(OC)c(N6CCC(=O)NC6=O)c5)CC4)CC3)c(Cl)c2)c2ccncc2c1=O] |
| E45 | 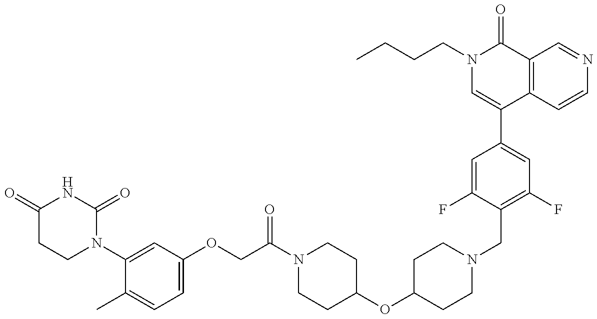 [SMILES: CCCCn1cc(-c2cc(F)c(CN3CCC(OC4CCN(C(=O)COc5ccc(C)c(N6CCC(=O)NC6=O)c5)CC4)CC3)c(F)c2)c2ccncc2c1=O] |
| E46 | 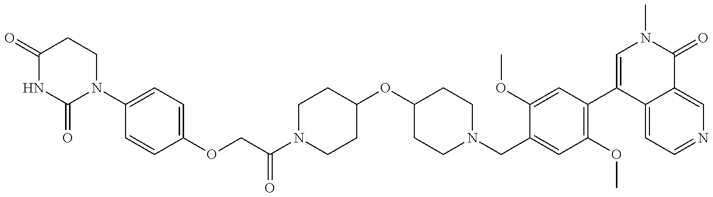 [SMILES: COc1cc(-c2cn(C)c(=O)c3cnccc23)c(OC)cc1CN1CCC(OC2CCN(C(=O)COc3ccc(N4CCC(=O)NC4=O)cc3)CC2)CC1] |
| E47 | 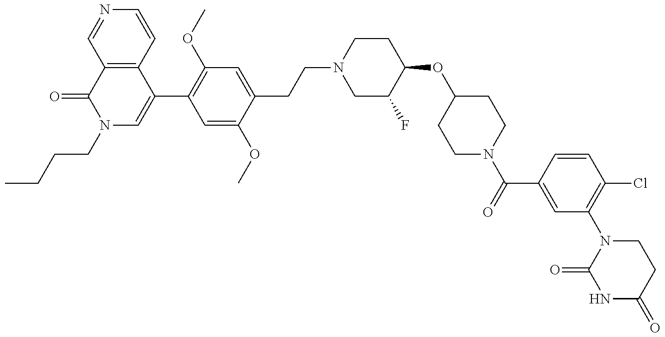 [SMILES: CCCCn1cc(-c2cc(OC)c(CCN3CC[C@@H](OC4CCN(C(=O)c5ccc(Cl)c(N6CCC(=O)NC6=O)c5)CC4)[C@H](F)C3)cc2OC)c2ccncc2c1=O] |
| E48 | 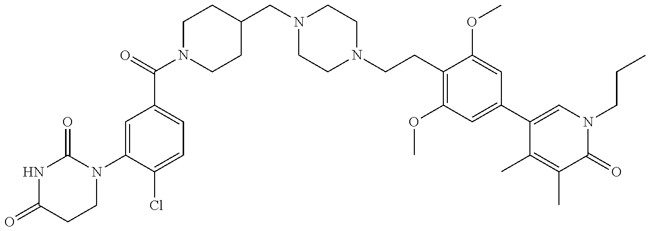 [SMILES: CCCn1cc(-c2cc(OC)c(CCN3CCN(CC4CCN(C(=O)c5ccc(Cl)c(N6CCC(=O)NC6=O)c5)CC4)CC3)c(OC)c2)c(C)c(C)c1=O] |
| E49 | 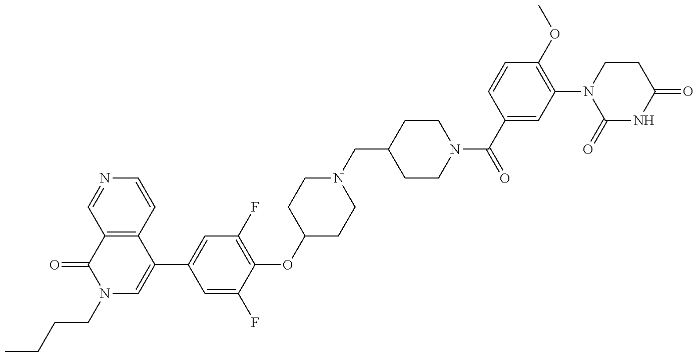 [SMILES: CCCCn1cc(-c2cc(F)c(OC3CCN(CC4CCN(C(=O)c5ccc(OC)c(N6CCC(=O)NC6=O)c5)CC4)CC3)c(F)c2)c2ccncc2c1=O] |
| E50 | 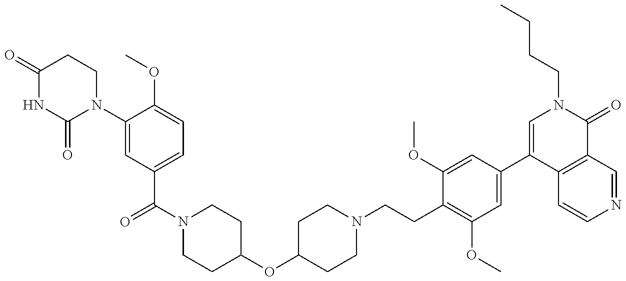 [SMILES: CCCCn1cc(-c2cc(OC)c(CCN3CCC(OC4CCN(C(=O)c5ccc(OC)c(N6CCC(=O)NC6=O)c5)CC4)CC3)c(OC)c2)c2ccncc2c1=O] |
| E51 | 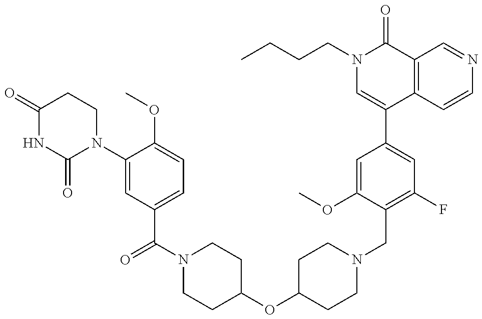 [SMILES: CCCCn1cc(-c2cc(F)c(CN3CCC(OC4CCN(C(=O)c5ccc(OC)c(N6CCC(=O)NC6=O)c5)CC4)CC3)c(OC)c2)c2ccncc2c1=O] |
| E52 | 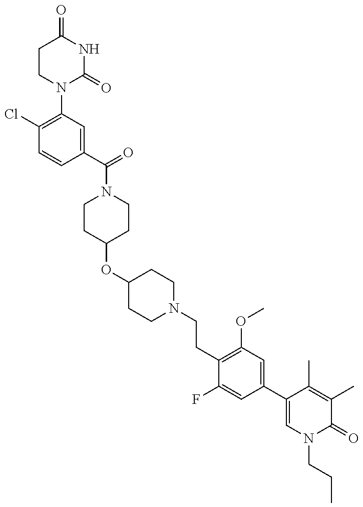 [SMILES: CCCn1cc(-c2cc(F)c(CCN3CCC(OC4CCN(C(=O)c5ccc(Cl)c(N6CCC(=O)NC6=O)c5)CC4)CC3)c(OC)c2)c(C)c(C)c1=O] |
| E53 | 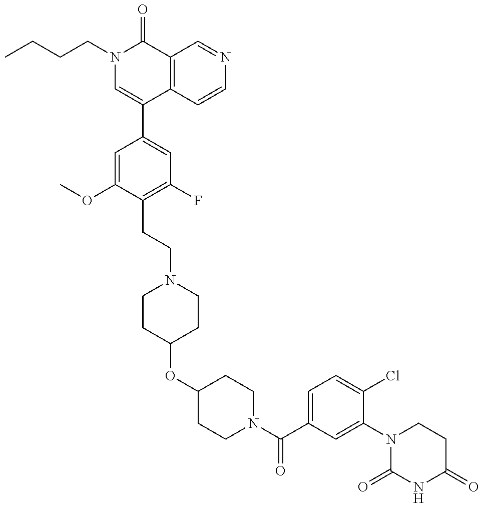 [SMILES: CCCCn1cc(-c2cc(F)c(CCN3CCC(OC4CCN(C(=O)c5ccc(Cl)c(N6CCC(=O)NC6=O)c5)CC4)CC3)c(OC)c2)c2ccncc2c1=O] |
| E54 | 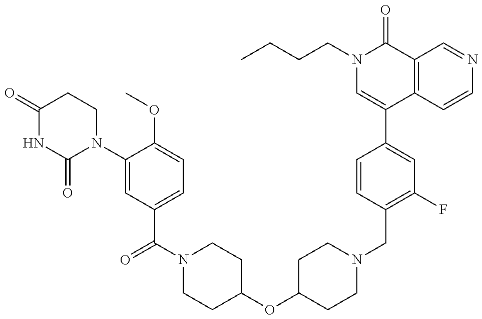 [SMILES: CCCCn1cc(-c2ccc(CN3CCC(OC4CCN(C(=O)c5ccc(OC)c(N6CCC(=O)NC6=O)c5)CC4)CC3)c(F)c2)c2ccncc2c1=O] |
| E55 | 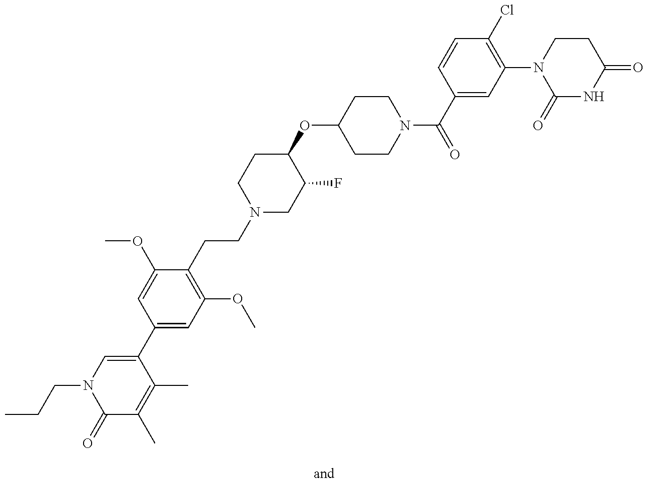 [SMILES: CCCn1cc(-c2cc(OC)c(CCN3CC[C@@H](OC4CCN(C(=O)c5ccc(Cl)c(N6CCC(=O)NC6=O)c5)CC4)[C@H](F)C3)c(OC)c2)c(C)c(C)c1=O] |
| E56 | 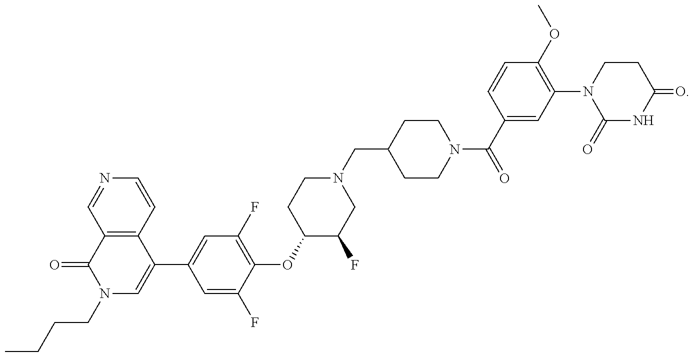 [SMILES: CCCCn1cc(-c2cc(F)c(O[C@@H]3CCN(CC4CCN(C(=O)c5ccc(OC)c(N6CCC(=O)NC6=O)c5)CC4)C[C@H]3F)c(F)c2)c2ccncc2c1=O] |

In an embodiment, the compound of a Formula above or a pharmaceutically acceptable salt, hydrate, solvate, prodrug, stereoisomer, or tautomer thereof, is selected from the group consisting of:

| Compound | |
|---|---|
| Number | Compound Structure |
| C1 | 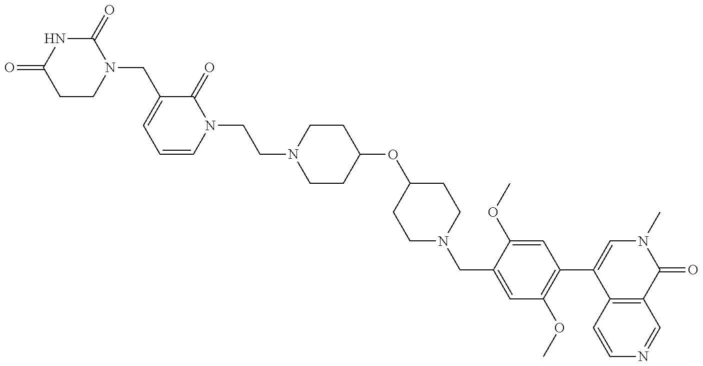 [SMILES: COc1cc(-c2cn(C)c(=O)c3cnccc23)c(OC)cc1CN1CCC(OC2CCN(CCn3cccc(CN4CCC(=O)NC4=O)c3=O)CC2)CC1] |
| C2 | 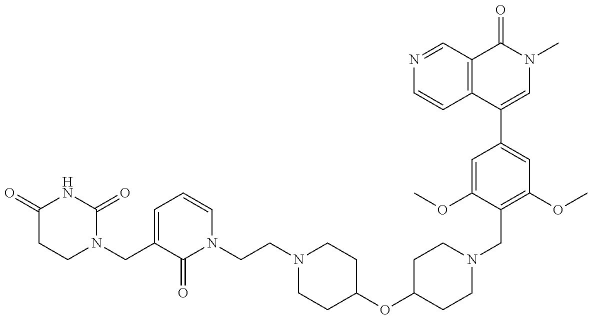 [SMILES: COc1cc(-c2cn(C)c(=O)c3cnccc23)cc(OC)c1CN1CCC(OC2CCN(CCn3cccc(CN4CCC(=O)NC4=O)c3=O)CC2)CC1] |
| C3 | 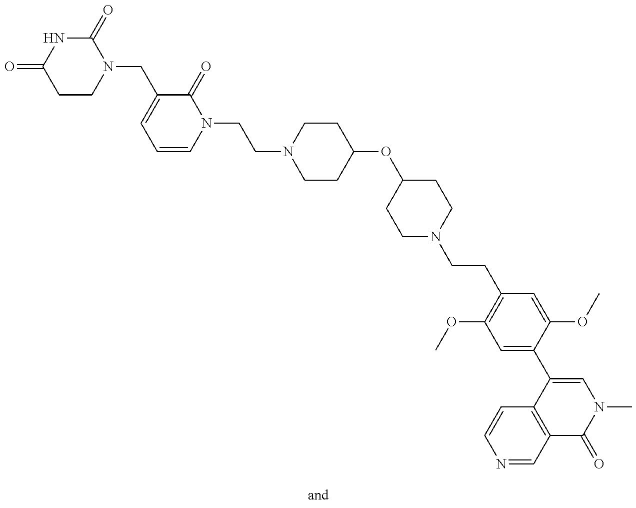 [SMILES: COc1cc(-c2cn(C)c(=O)c3cnccc23)c(OC)cc1CCN1CCC(OC2CCN(CCn3cccc(CN4CCC(=O)NC4=O)c3=O)CC2)CC1] |
| C4 | 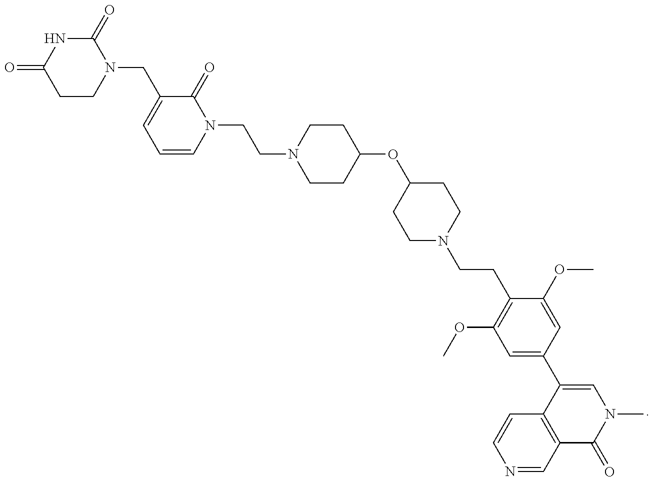 [SMILES: COc1cc(-c2cn(C)c(=O)c3cnccc23)cc(OC)c1CCN1CCC(OC2CCN(CCn3cccc(CN4CCC(=O)NC4=O)c3=O)CC2)CC1] |

In an embodiment, the compound of a Formula above or a pharmaceutically acceptable salt, hydrate, solvate, prodrug, stereoisomer, or tautomer thereof, is selected from the group consisting of:

| Compound | |
|---|---|
| Number | Compound Structure |
| D1 | 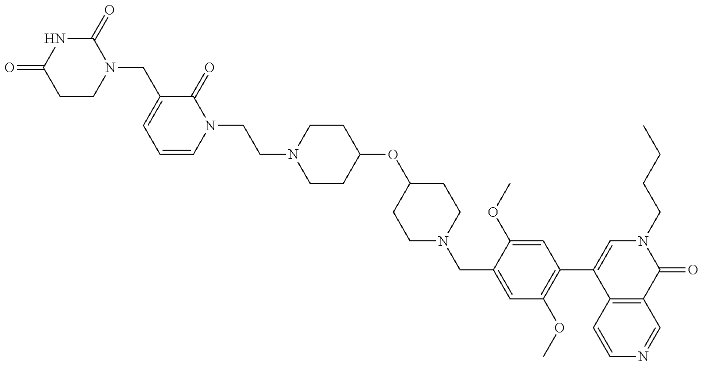 [SMILES: CCCCn1cc(-c2cc(OC)c(CN3CCC(OC4CCN(CCn5cccc(CN6CCC(=O)NC6=O)c5=O)CC4)CC3)cc2OC)c2ccncc2c1=O] |
| D2 | 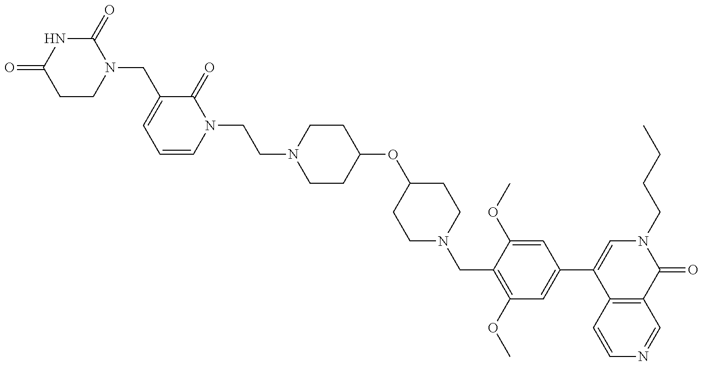 [SMILES: CCCCn1cc(-c2cc(OC)c(CN3CCC(OC4CCN(CCn5cccc(CN6CCC(=O)NC6=O)c5=O)CC4)CC3)c(OC)c2)c2ccncc2c1=O] |
| D3 | 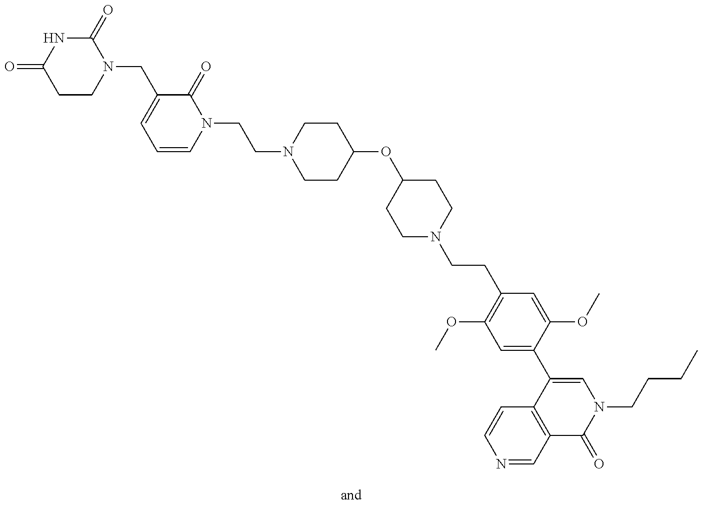 [SMILES: CCCCn1cc(-c2cc(OC)c(CCN3CCC(OC4CCN(CCn5cccc(CN6CCC(=O)NC6=O)c5=O)CC4)CC3)cc2OC)c2ccncc2c1=O] |
| D4 | 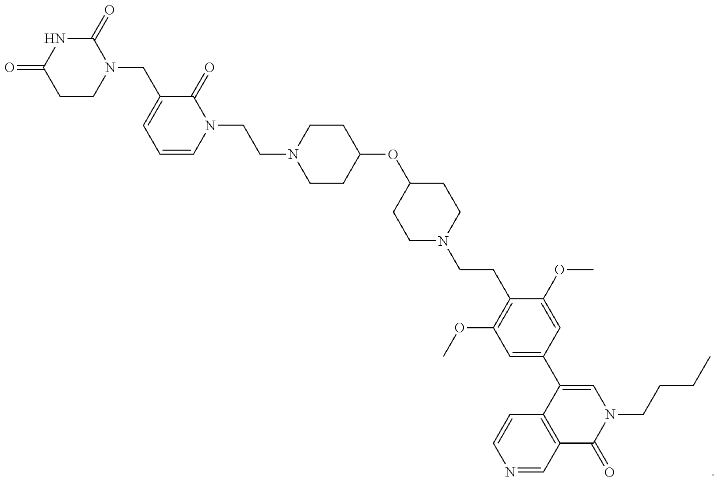 [SMILES: CCCCn1cc(-c2cc(OC)c(CCN3CCC(OC4CCN(CCn5cccc(CN6CCC(=O)NC6=O)c5=O)CC4)CC3)c(OC)c2)c2ccncc2c1=O] |

In an embodiment, the compound of a Formula above or a pharmaceutically acceptable salt, hydrate, solvate, prodrug, stereoisomer, or tautomer thereof, is selected from the group consisting of:

| Compound | |
|---|---|
| Number | Compound Structure |
| C1 | 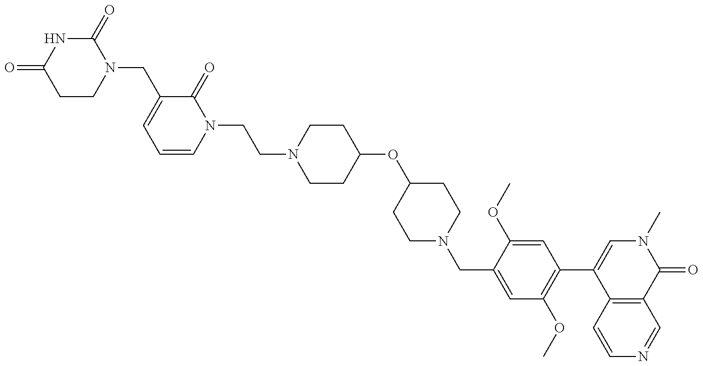 [SMILES: COc1cc(-c2cn(C)c(=O)c3cnccc23)c(OC)cc1CN1CCC(OC2CCN(CCn3cccc(CN4CCC(=O)NC4=O)c3=O)CC2)CC1] |
| C2 | 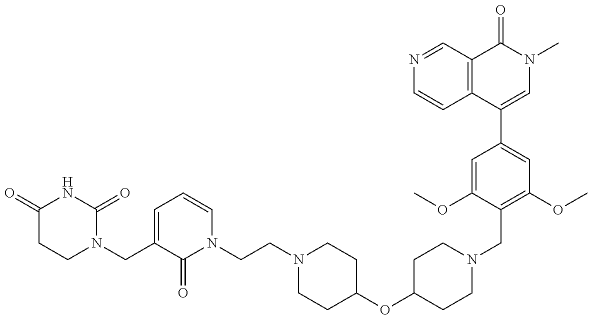 [SMILES: COc1cc(-c2cn(C)c(=O)c3cnccc23)cc(OC)c1CN1CCC(OC2CCN(CCn3cccc(CN4CCC(=O)NC4=O)c3=O)CC2)CC1] |
| C3 | 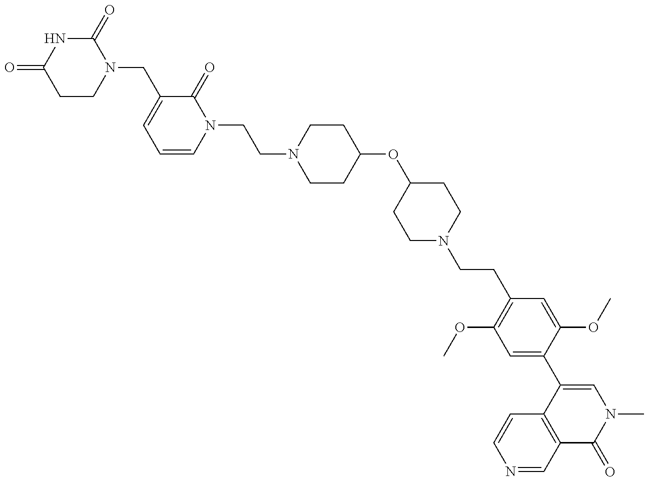 [SMILES: COc1cc(-c2cn(C)c(=O)c3cnccc23)c(OC)cc1CCN1CCC(OC2CCN(CCn3cccc(CN4CCC(=O)NC4=O)c3=O)CC2)CC1] |
| C4 | 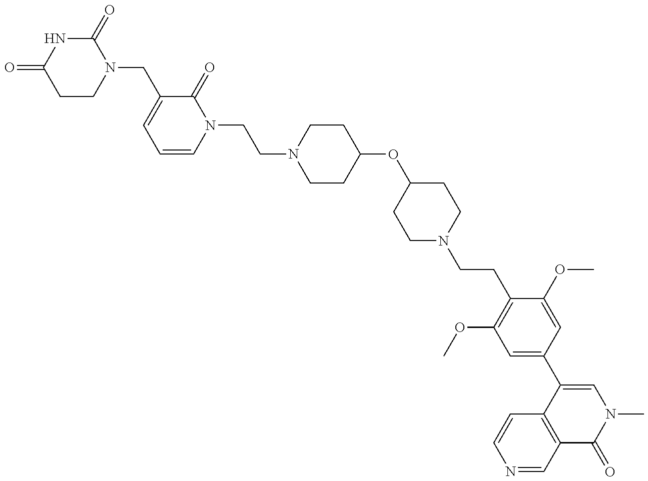 [SMILES: COc1cc(-c2cn(C)c(=O)c3cnccc23)cc(OC)c1CCN1CCC(OC2CCN(CCn3cccc(CN4CCC(=O)NC4=O)c3=O)CC2)CC1] |
| D1 | 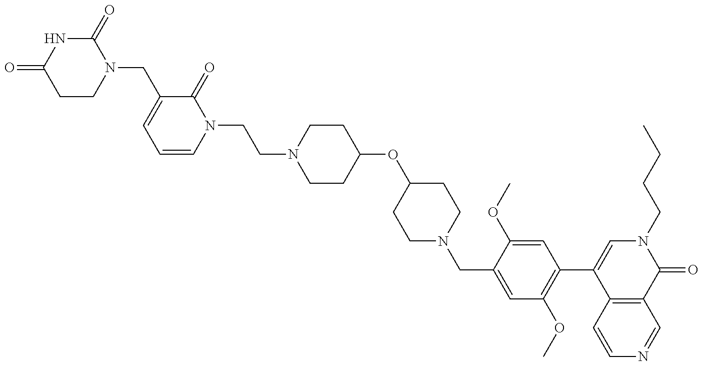 [SMILES: CCCCn1cc(-c2cc(OC)c(CN3CCC(OC4CCN(CCn5cccc(CN6CCC(=O)NC6=O)c5=O)CC4)CC3)cc2OC)c2ccncc2c1=O] |
| D2 | 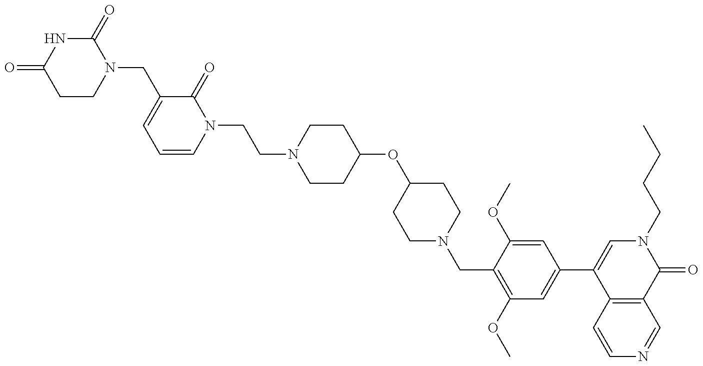 [SMILES: CCCCn1cc(-c2cc(OC)c(CN3CCC(OC4CCN(CCn5cccc(CN6CCC(=O)NC6=O)c5=O)CC4)CC3)c(OC)c2)c2ccncc2c1=O] |
| D3 | 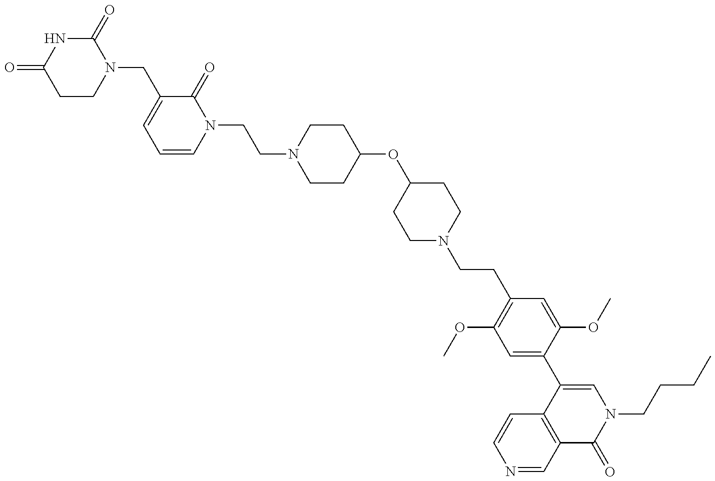 [SMILES: CCCCn1cc(-c2cc(OC)c(CCN3CCC(OC4CCN(CCn5cccc(CN6CCC(=O)NC6=O)c5=O)CC4)CC3)cc2OC)c2ccncc2c1=O] |
| D4 | 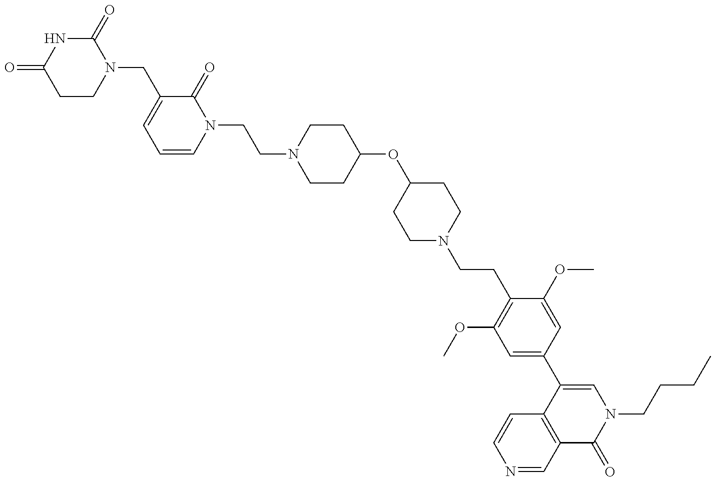 [SMILES: CCCCn1cc(-c2cc(OC)c(CCN3CCC(OC4CCN(CCn5cccc(CN6CCC(=O)NC6=O)c5=O)CC4)CC3)c(OC)c2)c2ccncc2c1=O] |
| E1 | 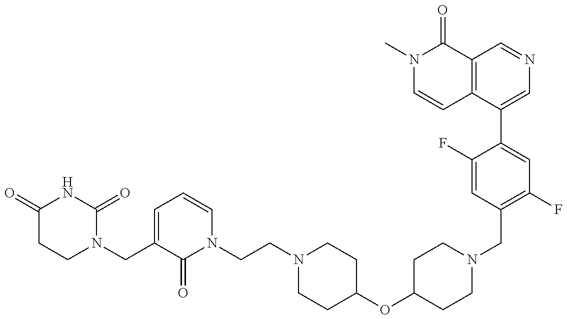 [SMILES: Cn1ccc2c(-c3cc(F)c(CN4CCC(OC5CCN(CCn6cccc(CN7CCC(=O)NC7=O)c6=O)CC5)CC4)cc3F)cncc2c1=O] |
| E2 | 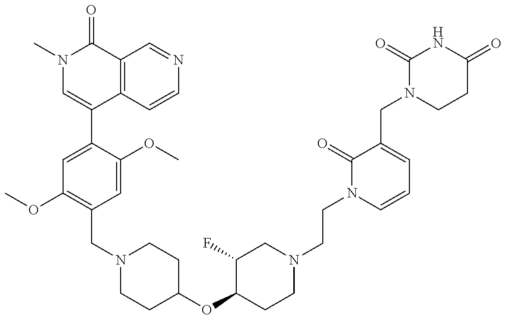 [SMILES: COc1cc(-c2cn(C)c(=O)c3cnccc23)c(OC)cc1CN1CCC(O[C@@H]2CCN(CCn3cccc(CN4CCC(=O)NC4=O)c3=O)C[C@H]2F)CC1] |
| E7 | 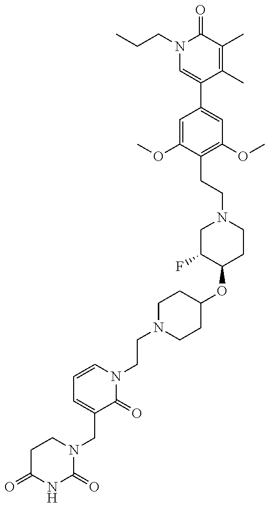 [SMILES: CCCn1cc(-c2cc(OC)c(CCN3CC[C@@H](OC4CCN(CCn5cccc(CN6CCC(=O)NC6=O)c5=O)CC4)[C@H](F)C3)c(OC)c2)c(C)c(C)c1=O] |
| E17 | 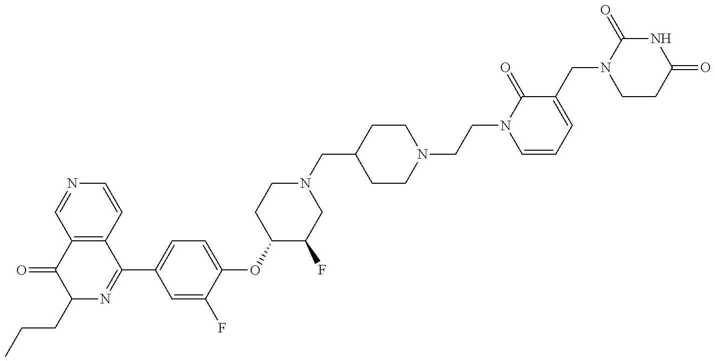 [SMILES: CCCC1N=C(c2ccc(O[C@@H]3CCN(CC4CCN(CCn5cccc(CN6CCC(=O)NC6=O)c5=O)CC4)C[C@H]3F)c(F)c2)c2ccncc2C1=O] |
| E18 | 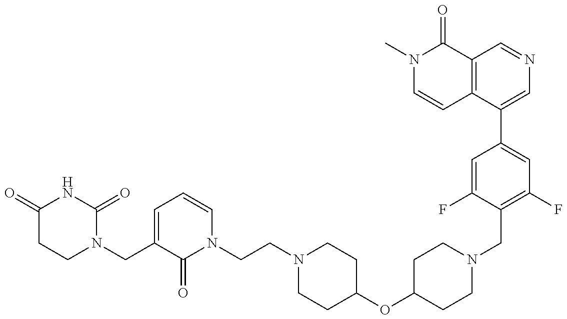 [SMILES: Cn1ccc2c(-c3cc(F)c(CN4CCC(OC5CCN(CCn6cccc(CN7CCC(=O)NC7=O)c6=O)CC5)CC4)c(F)c3)cncc2c1=O] |
| E23 | 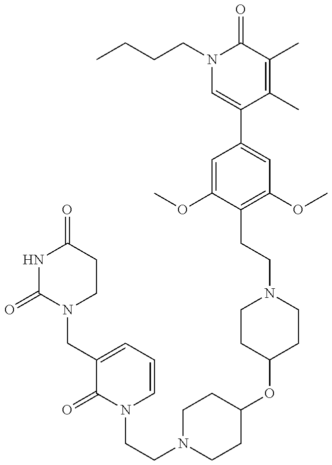 [SMILES: CCCCn1cc(-c2cc(OC)c(CCN3CCC(OC4CCN(CCn5cccc(CN6CCC(=O)NC6=O)c5=O)CC4)CC3)c(OC)c2)c(C)c(C)c1=O] |
| E24 | 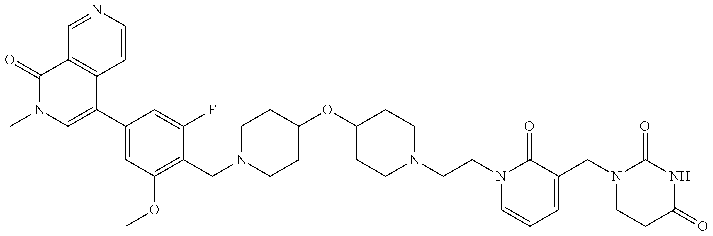 [SMILES: COc1cc(-c2cn(C)c(=O)c3cnccc23)cc(F)c1CN1CCC(OC2CCN(CCn3cccc(CN4CCC(=O)NC4=O)c3=O)CC2)CC1] |
| E27 | 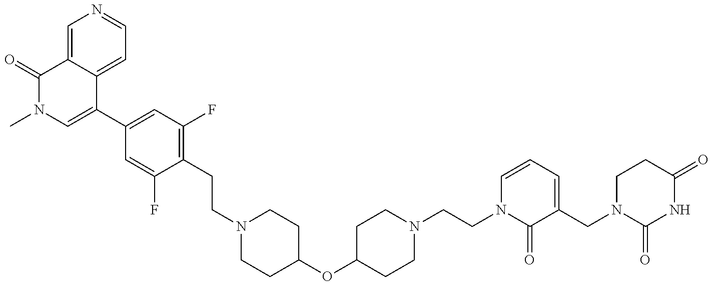 [SMILES: Cn1cc(-c2cc(F)c(CCN3CCC(OC4CCN(CCn5cccc(CN6CCC(=O)NC6=O)c5=O)CC4)CC3)c(F)c2)c2ccncc2c1=O] |
| E28 | 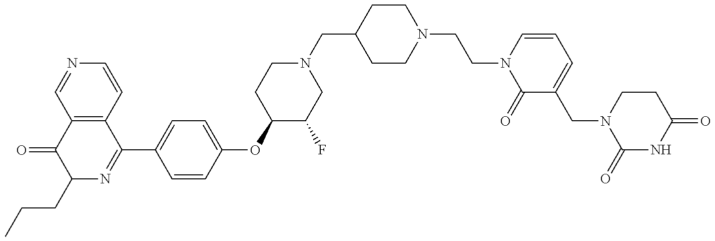 [SMILES: CCCC1N=C(c2ccc(O[C@H]3CCN(CC4CCN(CCn5cccc(CN6CCC(=O)NC6=O)c5=O)CC4)C[C@@H]3F)cc2)c2ccncc2C1=O] |
| E30 | 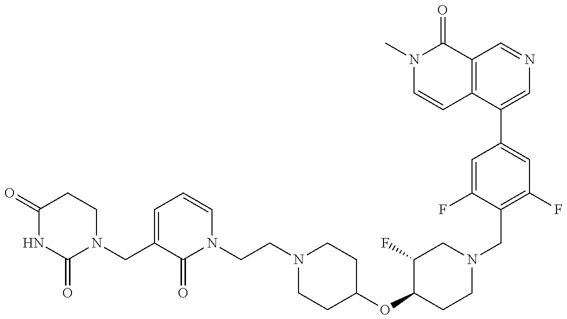 [SMILES: Cn1ccc2c(-c3cc(F)c(CN4CC[C@@H](OC5CCN(CCn6cccc(CN7CCC(=O)NC7=O)c6=O)CC5)[C@H](F)C4)c(F)c3)cncc2c1=O] |
| E35 | 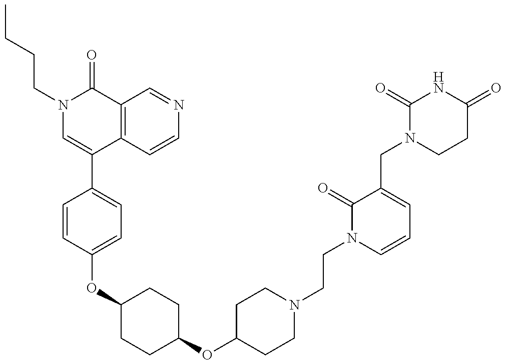 [SMILES: CCCCn1cc(-c2ccc(O[C@H]3CC[C@@H](OC4CCN(CCn5cccc(CN6CCC(=O)NC6=O)c5=O)CC4)CC3)cc2)c2ccncc2c1=O] |
| E37 | 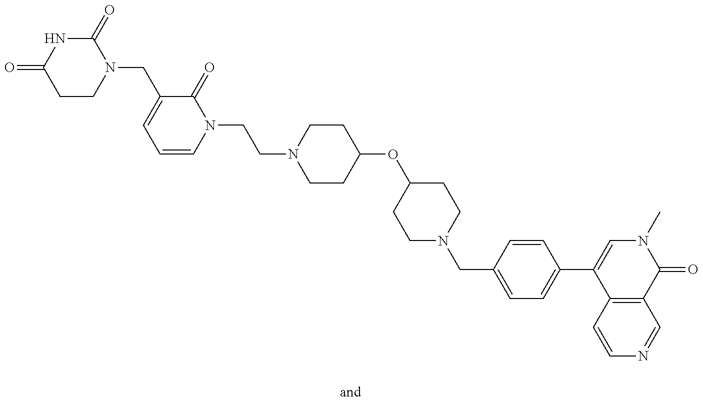 [SMILES: Cn1cc(-c2ccc(CN3CCC(OC4CCN(CCn5cccc(CN6CCC(=O)NC6=O)c5=O)CC4)CC3)cc2)c2ccncc2c1=O] |
| E39 | 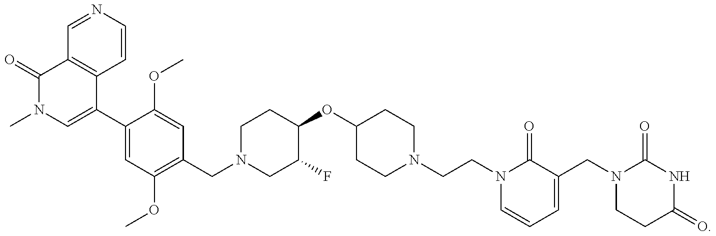 [SMILES: COc1cc(-c2cn(C)c(=O)c3cnccc23)c(OC)cc1CN1CC[C@@H](OC2CCN(CCn3cccc(CN4CCC(=O)NC4=O)c3=O)CC2)[C@H](F)C1] |

### Definitions

In an embodiment, a compound of any of the formulae described herein, e.g., a compound of Formula (A), (BF-I), (BF-II), (BF-II), (BF-IIIa), (BF-IIIb), (BF-IV), (BF-IVa), (BF-IVb), (BF-I′), (BF-II′), (BF-III′), (BF-IV′), or (BF-V′), or a pharmaceutically acceptable salt, hydrate, solvate, prodrug, stereoisomer, or tautomer thereof, that modulates, e.g., decreases the amount of a targeted protein, a bromodomain-containing protein, e.g., BRD9.

In an embodiment, a compound of any of the formulae described herein, e.g., a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), or (BF-V′), or a pharmaceutically acceptable salt, hydrate, solvate, prodrug, stereoisomer, or tautomer thereof, that degrades a targeted protein, e.g., a bromodomain-containing protein, e.g., BRD9, through the Ubiquitin-proteasome pathway (UPP).

The term “a therapeutically effective amount” of a compound of the disclosure refers to an amount of the compound of the disclosure that will elicit the biological or medical response of a subject, for example, reduction or inhibition of an enzyme or a protein activity, or ameliorate symptoms, alleviate conditions, slow or delay disease progression, or prevent a disease, etc. In an embodiment, the term “a therapeutically effective amount” refers to the amount of the compound of the disclosure that, when administered to a subject, is effective to (1) at least partially alleviate, prevent and/or ameliorate a condition, or a disorder or a disease (i) mediated by a bromodomain-containing protein, e.g., BRD9, or (ii) associated with activity of a bromodomain-containing protein, e.g., BRD9, or (iii) characterized by activity (normal or abnormal) of a bromodomain-containing protein, e.g., BRD9; or (2) reduce or inhibit the activity of a bromodomain-containing protein, e.g., BRD9; or (3) reduce or inhibit the expression of a bromodomain-containing protein, e.g., BRD9. These effects may be achieved for example by reducing the amount of a bromodomain-containing protein, e.g., BRD9, by degrading of the bromodomain-containing protein, e.g., BRD9. In an embodiment, the term “a therapeutically effective amount” refers to the amount of the compound of the disclosure that, when administered to a cell, or a tissue, or a non-cellular biological material, or a medium, is effective to at least partially reduce or inhibit the activity of a bromodomain-containing protein, e.g., BRD9; or at least partially reduce or inhibit the expression of a bromodomain-containing protein, e.g., BRD9, for example by degrading of a bromodomain-containing protein, e.g., BRD9.

As used herein, the terms “degrades”, “degrading”, or “degradation” refers to the partial or full breakdown of a target protein, e.g. a bromodomain-containing protein, e.g., BRD9, by the cellular proteasome system to an extent that reduces or eliminates the biological activity (especially aberrant activity) of a bromodomain-containing protein, e.g., BRD9. Degradation may be achieved through mediation of an E3 ligase, in particular, E3-ligase complexes comprising the protein Cereblon. As used herein, the term “modulation of BRD9 activity” or “modulating BRD9 activity” means the alteration of, especially reduction, suppression or elimination, of BRD9 activity. This may be achieved by degrading BRD9. The amount of BRD9 degraded can be measured by comparing the amount of BRD9 remaining after treatment with a compound of the disclosure as compared to the initial amount or level of BRD9 present as measured prior to treatment with a compound of the disclosure. In an embodiment, at least about 30% of BRD9 is degraded compared to initial levels. In an embodiment, at least about 40% of BRD9 is degraded compared to initial levels. In an embodiment, at least about 50% of BRD9 is degraded compared to initial levels. In an embodiment, at least about 60% of BRD9 is degraded compared to initial levels. In an embodiment, at least about 70% of BRD9 is degraded compared to initial levels. In an embodiment, at least about 80% of BRD9 is degraded compared to initial levels. In an embodiment, at least about 90% of BRD9 is degraded compared to initial levels. In an embodiment, at least about 95% of BRD9 is degraded compared to initial levels. In an embodiment, over 95% of BRD9 is degraded compared to initial levels. In an embodiment, at least about 99% of BRD9 is degraded compared to initial levels.

In an embodiment, the BRD9 is degraded in an amount of from about 30% to about 99% compared to initial levels. In an embodiment, the BRD9 is degraded in an amount of from about 40% to about 99% compared to initial levels. In an embodiment, the BRD9 is degraded in an amount of from about 50% to about 99% compared to initial levels. In an embodiment, the BRD9 is degraded in an amount of from about 60% to about 99% compared to initial levels. In an embodiment, the BRD9 is degraded in an amount of from about 70% to about 99% compared to initial levels. In an embodiment, the BRD9 is degraded in an amount of from about 80% to about 99% compared to initial levels. In an embodiment, the BRD9 is degraded in an amount of from about 90% to about 99% compared to initial levels. In an embodiment, the BRD9 is degraded in an amount of from about 95% to about 99% compared to initial levels. In an embodiment, the BRD9 is degraded in an amount of from about 90% to about 95% compared to initial levels.

As used herein, the term “selectivity for BRD9” means, for example, a compound of the disclosure degrades BRD9 in preference to, or to a greater extent than, another protein or proteins.

As used herein, the term “subject” refers to an animal. Typically, the animal is a mammal. A subject also refers to, for example, primates (e.g., humans, male or female), cows, sheep, goats, horses, dogs, cats, rabbits, rats, mice, fish, birds and the like. In an embodiment, the subject is a primate. In a preferred embodiment, the subject is a human.

As used herein, the terms “inhibit”, “inhibition”, or “inhibiting” refer to the reduction or suppression of a given condition, symptom, or disorder, or disease, or a significant decrease in the baseline activity of a biological activity or process.

As used herein, the terms “treat”, “treating”, or “treatment” of any disease or disorder refer in an embodiment, to ameliorating the disease or disorder (i.e., slowing or arresting or reducing the development of the disease or at least one of the clinical symptoms thereof). In another embodiment, “treat”, “treating”, or “treatment” refers to alleviating or ameliorating at least one physical parameter including those which may not be discernible by the patient.

As used herein, the term “preventing” refers to a reduction in the frequency of, or delay in the onset of, symptoms of the condition or disease.

As used herein, a subject is “in need of” a treatment if such subject would benefit biologically, medically, or in quality of life from such treatment.

As used herein, the term “a,” “an,” “the” and similar terms used in the context of the disclosure (especially in the context of the claims) are to be construed to cover both the singular and plural unless otherwise indicated herein or clearly contradicted by the context.

The term “alkyl” refers to a radical of a straight-chain or branched saturated hydrocarbon group having from 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C2), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tertiary amyl), and hexyl (C₆) (e.g., n-hexyl).

“Alkylene” refers to a divalent radical of an alkyl group, e.g., —CH₂—, —CH₂CH₂—, and —CH₂CH₂CH₂—.

“Alkenyl” refers to a radical of a straight-chain or branched hydrocarbon group having from 2 to 6 carbon atoms and one or more carbon-carbon double bonds (e.g., 1, 2, 3, or 4 double bonds). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₆ alkenyl. In certain embodiments, the alkenyl group is a substituted C₂₋₆ alkenyl. In an alkenyl group, a C═C double bond for which the stereochemistry is not specified (e.g., —CH═CHCH₃ or

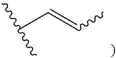

may be an (E)- or (Z)-double bond.

“Alkenylene” refers to a divalent radical of an alkenyl group, e.g., —CH═CH₂—, —CH═CH₂CH₂—, and —CH═CH₂CH₂CH₂—.

“Heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

“Heteroalkylene” refers to a divalent radical of a heteroalkyl group.

“Alkoxy” or “alkoxyl” refers to an —O-alkyl radical. In some embodiments, the alkoxy groups are methoxy, ethoxy, n-propoxy, isopropoxy, n-butoxy, tert-butoxy, sec-butoxy, n-pentoxy, n-hexoxy, and 1,2-dimethylbutoxy. In some embodiments, alkoxy groups are lower alkoxy, i.e., with between 1 and 6 carbon atoms. In some embodiments, alkoxy groups have between 1 and 4 carbon atoms.

As used herein, the term “aryl” refers to a stable, aromatic, mono- or bicyclic ring radical having the specified number of ring carbon atoms. Examples of aryl groups include, but are not limited to, phenyl, 1-naphthyl, 2-naphthyl, and the like. The related term “aryl ring” likewise refers to a stable, aromatic, mono- or bicyclic ring having the specified number of ring carbon atoms. In an embodiment, aryl is phenyl.

As used herein, the term “heteroaryl” refers to a stable, aromatic, mono- or bicyclic ring radical having the specified number of ring atoms and comprising one or more heteroatoms individually selected from nitrogen, oxygen and sulfur. The heteroaryl radical may be bonded via a carbon atom or heteroatom. Examples of heteroaryl groups include, but are not limited to, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl, pyridyl, quinolinyl, isoquinolinyl, indolyl, indazolyl, oxadiazolyl, benzothiazolyl, quinoxalinyl, and the like. The related term “heteroaryl ring” likewise refers to a stable, aromatic, mono- or bicyclic ring having the specified number of ring atoms and comprising one or more heteroatoms individually selected from nitrogen, oxygen and sulfur.

As used herein, the term “carbocyclyl” refers to a stable, saturated or unsaturated, non-aromatic, mono- or bicyclic (fused, bridged, or spiro) ring radical having the specified number of ring carbon atoms. Examples of carbocyclyl groups include, but are not limited to, the cycloalkyl groups identified above, cyclobutyl, cyclopentyl, cyclohexyl, and the like. The related term “carbocyclic ring” likewise refers to a stable, saturated or unsaturated, non-aromatic, mono- or bicyclic (fused, bridged, or spiro) ring having the specified number of ring carbon atoms.

As used herein, the term “heterocyclyl” refers to a stable, saturated or unsaturated, non-aromatic, mono- or bicyclic (fused, bridged, or spiro) ring radical having the specified number of ring atoms and comprising one or more heteroatoms individually selected from nitrogen, oxygen and sulfur. The heterocyclyl radical may be bonded via a carbon atom or heteroatom. Examples of heterocyclyl groups include, but are not limited to, azetidinyl, oxetanyl, pyrrolinyl, pyrrolidinyl, tetrahydrofuryl, tetrahydrothienyl, piperidyl, piperazinyl, tetrahydropyranyl, morpholinyl, perhydroazepinyl, tetrahydropyridinyl, tetrahydroazepinyl, octahydropyrrolopyrrolyl, and the like. The related term “heterocyclic ring” likewise refers to a stable, saturated or unsaturated, non-aromatic, mono- or bicyclic (fused, bridged, or spiro) ring having the specified number of ring atoms and comprising one or more heteroatoms individually selected from nitrogen, oxygen and sulfur.

“Spirocycloalkyl” or “spirocyclyl” means carbogenic bicyclic ring systems with both rings connected through a single atom. The rings can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. For example, a (C₃-C₁₂)spirocycloalkyl is a spirocycle containing between 3 and 12 carbon atoms.

“Spiroheterocycloalkyl” or “spiroheterocyclyl” means a spirocycle wherein at least one of the rings is a heterocycle wherein one or more of the carbon atoms can be substituted with a heteroatom (e.g., one or more of the carbon atoms can be substituted with a heteroatom in at least one of the rings). One or both of the rings in a spiroheterocycle can be fused to another ring carbocyclic, heterocyclic, aromatic, or heteroaromatic ring.

“Halo” or “halogen” refers to fluorine (fluoro, —F), chlorine (chloro, —Cl), bromine (bromo, —Br), or iodine (iodo, —I).

“Haloalkyl” means an alkyl group substituted with one or more halogens. Examples of haloalkyl groups include, but are not limited to, trifluoromethyl, difluoromethyl, pentafluoroethyl, and trichloromethyl.

“Substituted”, whether preceded by the term “optionally” or not, means that one or more hydrogens of the designated moiety are replaced with a suitable substituent.

As used herein, the definition of each expression, e.g., alkyl, m, n, etc., when it occurs more than once in any structure, is intended to be independent of its definition elsewhere in the same structure.

Various embodiments of the disclosure are described herein. It will be recognized that features specified in each embodiment may be combined with other specified features, including as indicated in the embodiments below, to provide further embodiments of the present disclosure.

It is understood that in the following embodiments, combinations of substituents or variables of the depicted formulae are permissible only if such combinations result in stable compounds.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Certain compounds of the disclosure may exist in particular geometric or stereoisomeric forms. If, for instance, a particular enantiomer of a compound of the disclosure is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Unless otherwise stated, structures depicted herein are also meant to include geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the disclosed compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the disclosed structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the disclosure.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer.

ee=(90−10)/100*100=80%.

Thus, a composition containing 90% of one enantiomer and 10% of the other enantiomer is said to have an enantiomeric excess of 80%. The compounds or compositions described herein may contain an enantiomeric excess of at least 50%, 75%, 90%, 95%, or 99% of one form of the compound, e.g., the S-enantiomer. In other words such compounds or compositions contain an enantiomeric excess of the S enantiomer over the R enantiomer.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, Ind. 1972). All methods described herein can be performed in any suitable order unless otherwise indicated herein or otherwise clearly contradicted by context. The use of any and all examples, or exemplary language (e.g. “such as”) provided herein is intended merely to better illuminate the disclosure and does not pose a limitation on the scope of the disclosure otherwise claimed.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the disclosure into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

### Pharmaceutically Acceptable Salts

Pharmaceutically acceptable salts of these compounds are also contemplated for the uses described herein. As used herein, the terms “salt” or “salts” refer to an acid addition or base addition salt of a compound of the disclosure. “Salts” include in particular “pharmaceutical acceptable salts.” The term “pharmaceutically acceptable salts” refers to salts that retain the biological effectiveness and properties of the compounds disclosed herein and, which typically are not biologically or otherwise undesirable. In many cases, the compounds disclosed herein are capable of forming acid and/or base salts by virtue of the presence of amino and/or carboxyl groups or groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium, and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, and tromethamine.

In another aspect, the disclosure provides a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), or (BF-V′) in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate, or xinafoate salt form.

### Pharmaceutical Compositions

In another aspect, the disclosure provides a pharmaceutical composition comprising one or more compounds of described herein or a pharmaceutically acceptable salt, hydrate, solvate, prodrug, stereoisomer, or tautomer thereof, and one or more pharmaceutically acceptable carrier. The term “pharmaceutically acceptable carrier” refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compositions of the disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrastemal, intrathecal, intrahepatic, intralesional and intracranial injection or infusion techniques. In some embodiments, the compositions of the disclosure are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tween®, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax, and polyethylene glycols.

The pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

The pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. The amount of the compounds of the present disclosure that may be combined with the carrier materials to produce a composition in a single dosage form will vary depending upon the host treated, the particular mode of administration. Preferably, the compositions should be formulated so that a dosage of between 0.01-100 mg/kg body weight/day of the compound can be administered to a patient receiving these compositions.

### Isotopically Labelled Compounds

A compound of described herein or a pharmaceutically acceptable salt, hydrate, solvate, prodrug, stereoisomer, or tautomer thereof, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, 35, ³⁶Cl, ¹²³I, ¹²⁴I, ¹²⁵I, respectively. The disclosure includes various isotopically labeled compounds as defined herein, for example, those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example 2H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the disclosure or a pharmaceutically acceptable salt, hydrate, solvate, prodrug, stereoisomer, or tautomer thereof, can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of a compound of the disclosure or a pharmaceutically acceptable salt, hydrate, solvate, prodrug, stereoisomer, or tautomer thereof. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Dosages

Toxicity and therapeutic efficacy of compounds of the disclosure, including pharmaceutically acceptable salts and deuterated variants, can be determined by standard pharmaceutical procedures in cell cultures or experimental animals. The LD₅₀ is the dose lethal to 50% of the population. The ED₅₀ is the dose therapeutically effective in 50% of the population. The dose ratio between toxic and therapeutic effects (LD₅₀/ED₅₀) is the therapeutic index. Compounds that exhibit large therapeutic indexes are preferred. While compounds that exhibit toxic side effects may be used, care should be taken to design a delivery system that targets such compounds to the site of affected tissue in order to minimize potential damage to uninfected cells and thereby reduce side effects.

Data obtained from the cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Methods of Use

In another aspect, the disclosure provides a method of treating or preventing a disorder in a subject in need thereof, the method comprising administering to the subject a therapeutically effective amount of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof.

In another aspect, the disclosure provides a method of treating or preventing a disorder mediated by a bromodomain protein, e.g., BRD9, in a subject in need thereof, the method comprising administering to the subject a therapeutically effective amount of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof.

In another aspect, the disclosure provides a method of modulating bromodomain-containing protein 9 (BRD9) in a subject in need thereof, the method comprising administering to the subject a therapeutically effective amount of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof.

In another aspect, the disclosure provides a method of inhibiting bromodomain-containing protein 9 (BRD9) in a subject in need thereof, the method comprising administering to the subject a therapeutically effective amount of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof.

In another aspect, the disclosure provides a method for inducing degradation of a bromodomain protein, e.g., BRD9, in a subject in need thereof, the method comprising administering to the subject a therapeutically effective amount of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof.

In another aspect, the disclosure provides a method of inhibiting, reducing, or eliminating the activity of a bromodomain protein, e.g., BRD9, the method comprising administering to the subject a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof.

In an embodiment, inhibiting, reducing, or eliminating the activity of a bromodomain protein, e.g., BRD9, comprises recruiting a ligase (e.g., Cereblon E3 Ubiquitin ligase) with the Targeting Ligase Binder, e.g., a Targeting Ligase Binder described herein, of the compound, e.g., a compound of Formula (A), (BF-I), (BF-II), (BF-III), (BF-IIIa), (BF-IIIb), (BF-IV), (BF-IVa), (BF-IVb), or (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, forming a ternary complex of the bromodomain protein, e.g., BRD9, the compound, and the ligase, to thereby inhibit, reduce or eliminate the activity of the bromodomain protein, e.g., BRD9 protein.

In another aspect, the disclosure provides a method of treating or preventing a cancer in a subject in need thereof, the method comprising administering to the subject a therapeutically effective amount of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof.

In another aspect, the disclosure provides a method of treating or preventing a cancer mediated by a bromodomain protein, e.g., BRD9, in a subject in need thereof, the method comprising administering to the subject a therapeutically effective amount of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof.

In another aspect, the disclosure provides a method of treating a disorder selected from an inflammatory, an autoimmune, a cardiovascular, a neurodegenerative, liver disorder, kidney disorder, viral or bacterial infection, and a bone disorder, in a subject in need thereof, the method comprising administering to the subject a therapeutically effective amount of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof.

In another aspect, the disclosure provides compounds of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof, for use in modulating bromodomain-containing protein 9 (BRD9) in a subject in need thereof.

In another aspect, the disclosure provides compounds of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof, for use in inhibiting bromodomain-containing protein 9 (BRD9) in a subject in need thereof.

In another aspect, the disclosure provides compounds of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof, for use in treating or preventing a cancer in a subject in need thereof.

In another aspect, the disclosure provides compounds of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof, for use in treating or preventing a cancer mediated by a bromodomain protein, e.g., BRD9, in a subject in need thereof.

In another aspect, the disclosure provides compounds of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof, for use in treating a disorder selected from an inflammatory, an autoimmune, a cardiovascular, a neurodegenerative, liver disorder, kidney disorder, viral or bacterial infection, and a bone disorder, in a subject in need thereof.

In another aspect, the disclosure provides a use of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof, in the manufacture of a medicament for modulating bromodomain-containing protein 9 (BRD9) in a subject in need thereof.

In another aspect, the disclosure provides a use of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof, in the manufacture of a medicament for inhibiting bromodomain-containing protein 9 (BRD9) in a subject in need thereof.

In another aspect, the disclosure provides a use of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof, in the manufacture of a medicament for treating or preventing a cancer mediated by a bromodomain protein, e.g., BRD9, in a subject in need thereof.

In another aspect, the disclosure provides a use of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof, in the manufacture of a medicament for treating or preventing a cancer in a subject in need thereof.

In another aspect, the disclosure provides a use of a compound of Formula (A), (BF-I), (BF-II), (BF-III), (BF-IIIa), (BF-IIIb), (BF-IV), (BF-IVa), (BF-IVb), (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof, in the manufacture of a medicament for treating a disorder selected from an inflammatory, an autoimmune, a cardiovascular, a neurodegenerative, liver disorder, kidney disorder, viral or bacterial infection, and a bone disorder, in a subject in need thereof.

### Diseases and Disorders

In an embodiment, the compounds described herein can be used to treat the following diseases and disorders.

Cancers

In an embodiment, the cancer is selected from lung cancer, colon cancer, colorectal cancer, breast cancer, prostate cancer, liver cancer, pancreatic cancer, brain cancer, kidney cancer, ovarian cancer, stomach cancer, cervical cancer, skin cancer, basal cell carcinoma, adenocarcinoma, gastrointestinal cancer, lip cancer, bone cancer, mouth cancer, esophageal cancer, small bowel cancer, gastric cancer, breast cancer, glioma, glioblastoma, hepatocellular carcinoma, renal cell carcinoma, papillary renal carcinoma, squamous cell and/or basal cell cancers, head and neck squamous cell carcinoma, leukemias, lymphomas, myelomas, or solid tumors.

In an embodiment, the cancer is sarcoma. In an embodiment, the cancer is sarcoma of the bones, muscles, tendons, cartilage, nerves, fat, or blood vessels. In an embodiment, the cancer is soft tissue sarcoma, bone sarcoma, or osteosarcoma. In an embodiment, the cancer is angiosarcoma, fibrosarcoma, liposarcoma, leiomyosarcoma, Karposi's sarcoma, osteosarcoma, gastrointestinal stromal tumor, Synovial sarcoma, Pleomorphic sarcoma, chondrosarcoma, Ewing's sarcoma, reticulum cell sarcoma, meningiosarcoma, botryoid sarcoma, rhabdomyosarcoma, or embryonal rhabdomyosarcoma.

In an embodiment, the cancer is multiple myeloma.

In an embodiment, the cancer is epithelial call-derived neoplasia (epithelial carcinoma).

In an embodiment, the cancer is a cancer that affects epithelial cells throughout the body, such as, chronic myelogenous leukemia (CML), acute myeloid leukemia (AML) and acute promyelocytic leukemia (APL).

BRD9 and BAF (SWI/SNF)-Mediated Cancers

In an embodiment, the cancer is mediated by BRD9. In an embodiment, the cancer is mediated by the BAF (SWI/SNF) complex. In an embodiment, the cancer is selected from colorectal cancer, ovarian cancer, pancreatic cancer, renal cell carcinoma, hepatocellular carcinoma, bladder cancer, gastric cancer, breast cancer, glioma, medulloblastoma, squamous cell carcinoma, melanoma, lung, acute myeloid leukemia, synovial sarcoma, chronic lymphocytic leukemia, diffuse large B-cell lymphoma, non-Hodgkin lymphoma, Burkitt lymphoma, multiple myeloma, T-lineage acute lymphoblastic leukemia, clear-cell ovarian cancer, adenoid cystic carcinoma and malignant rhabdoid tumor.

Inflammation and Autoimmune

In an embodiment, the disease or disorder is an inflammatory disease. In an embodiment, the disease or disorder is an autoimmune disorder. In an embodiment, the disease or disorder is an autoinflammatory disorder. In an embodiment, the disease or disorder is selected from arthritis, rheumatoid arthritis, spondyiarthropathies, gouty arthritis, osteoarthritis, juvenile arthritis, and other arthritic conditions, multiple sclerosis, systemic lupus erthematosus (SLE), skin-related conditions, psoriasis, eczema, burns, dermatitis, neuroinflammation, allergy, pain, neuropathic pain, fever, pulmonary disorders, lung inflammation, adult respiratory distress syndrome, pulmonary sarcoisosis, asthma, silicosis, chronic pulmonary inflammatory disease, chronic obstructive pulmonary disease (COPD), gastrointestinal conditions, inflammatory bowel disease, Crohn's disease, gastritis, irritable bowel syndrome, ulcerative colitis, ulcerative diseases, and gastric ulcers.

Cardiovascular

In an embodiment, the disease or disorder is a cardiovascular disease, arteriosclerosis, myocardial infarction (including post-myocardial infarction indications), thrombosis, congestive heart failure, cardiac reperfusion injury, as well as complications associated with hypertension and/or heart failure such as vascular organ damage, restenosis, cardiomyopathy, stroke including ischemic and hemorrhagic stroke, reperfusion injury, renal reperfusion injury, ischemia including stroke and brain ischemia, and ischemia resulting from cardiac/coronary bypass.

Neurodegenerative Disorders

In an embodiment, the disorder is a neurodegenerative disorder, e.g., Alzheimer disease, Parkinson disease, Huntington's disease, amyotrophic lateral sclerosis, spinal cord injury, peripheral neuropathy, Coffin-Siris syndrome, Nicolaides-Baraitser syndrome, Kleefstra's syndrome, or autism spectrum disorders.

Liver and Kidney Diseases

In an embodiment, the disorder is a liver or kidney disease, e.g., nephritis.

Viral and Bacterial Infections

In an embodiment, the disease or disorder is a viral or bacterial infection. In an embodiment, the disease or disorder is selected from sepsis, septic shock, gram negative sepsis, malaria, meningitis, HIV infection, opportunistic infections, cachexia secondary to infection or malignancy, cachexia secondary to acquired immune deficiency syndrome (AIDS), AIDS, ARC (AIDS related complex), pneumonia, herpes virus, myalgias due to infection, influenza, graft vs. host reaction, and allograft rejections.

In an embodiment, the disease or disorder is a viral infection with a virus of the Retroviridae family, Hepadnaviridae family, Flaviviridae family, Adenoviridae family, Herpesviridae family, Papillomaviridae family, Parvoviridae family, Polyomaviridae family, Paramyxoviridae family, or Togaviridae family.

Bone Disorders

In an embodiment, the disease or disorder is a bone resorption disease, and osteoporosis.

### Combination Therapy

In another aspect, the disclosure provides a pharmaceutical combination comprising a compound of Formula (A), (BF-I), (BF-II), (BF-III), (BF-IIIa), (BF-IIIb), (BF-IV), (BF-IVa), (BF-IVb) (BF-I′), (BF-II′), (BF-III′), (BF-IV′), (BF-V′), or Compounds A1 to A42, B1 to B10, C1 to C4, D1 to D4, E1 to E7, E12 to E18, E22 to E25, E27 to E37, E39, E40, E42, E43, E45 to E56, or a pharmaceutically acceptable salt, hydrate, solvate, prodrug, stereoisomer, or tautomer thereof, and one or more additional therapeutic agent(s) for simultaneous, separate or sequential use in therapy.

### Methods of Making

The compounds of the disclosure can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art. Preferred methods include but are not limited to those methods described below.

The disclosed compounds may be synthesized according to the general methods described in the following synthetic schemes 1, 1a, 2-12, 12a, 12b, 12c, 13-16, 16a, and 17-21 wherein R¹, R², R³, R⁴, R^{4′}, R⁵, X¹, X², L¹, L², L³, m and n are as described herein. Starting materials are either commercially available or made by known procedures in the reported literature or as illustrated.

Compounds of Formula (BF-III), wherein X¹ is a nitrogen-containing heterocyclyl, e.g., a piperidinyl or piperazinyl and R¹, R², R³, R⁴, R⁵, X², L¹, L², L³, m and n are as previously defined, may be made according to Scheme 1. Thus, reaction of a compound of Formula (II) and a compound of Formula (III), for example in a reductive amination reaction, using conditions such as ZnCl₂ and NaBH₃CN, in a solvent mixture such as THF/DMSO and MeOH, provides a compound of Formula (BF-III). Alternative conditions for the reductive amination reaction are NaOAc, HOAc, and NaBH(OAc)₃ in DCM. L^{1a} is defined as a linker that is shorter by a single methylene group than L¹, where the formula of L¹ allows (e.g. in an embodiment where L¹ is —CH₂CH₂—, then L^{1a} is —CH₂—). Suitable L¹ include C₁₋₆ alkylene and C₁₋₆ heteroalkylene.

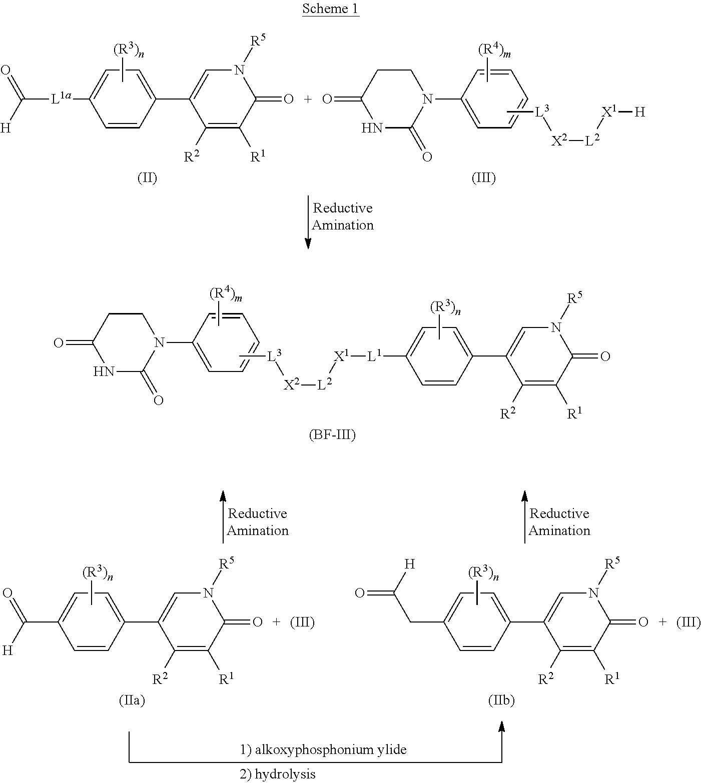

In an embodiment, specific examples of compounds of Formula (II) may include (IIa) and (IIb); both (IIa) and (IIb) can react similarly in a reductive amination reaction with compounds of Formula (III) to provide a compound of Formula (BF-III). In an embodiment, compounds of Formula (II) with longer chain extension can be made, for example, by conversion of (IIa) into (IIb). This can be achieved, for example, by reacting (IIa) with an alkoxyphosphonium ylide, such as that derived from (methoxymethyl)triphenylphosphonium halide and a base (e.g., potassium tert-butoxide (KO^{t}Bu), etc.), in a solvent (e.g., tetrahydrofuran (THF), diethyl ether (Et₂O), etc.) to produce an enol ether which is then hydrolyzed in a second step.

In an embodiment, compounds of Formula (BF-IIIc), wherein X¹ is piperidinyl, L¹ is C₁₋₆ alkylene, and R¹, R², R³, R⁴, R⁵, X², L², L³, m and n are as already defined herein above, may be made according to Scheme 1a. For example, reductive amination of a compound of Formula (II) and a compound of Formula (IIIa) using conditions such as ZnCl₂ and NaBH₃CN, in a solvent mixture, such as THF/DMSO and MeOH, provides a compound of Formula (BF-IIIc).

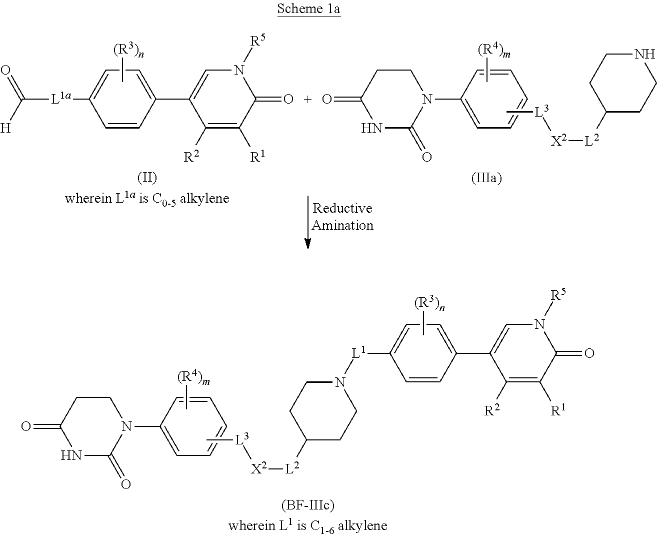

Alternatively, compounds of Formula (BF-III) may be synthesized from a compound of Formula (IV), wherein X² is a nitrogen-containing heterocycle, e.g. a piperidinyl or a piperizanyl, and a compound of Formula (V) according to Scheme 2, in an amide coupling reaction, using an amide coupling reagent such as HATU, in a solvent such as DMF, in the presence of a base such as DIPEA. L³a is defined as the subset of linkers L³, that contain a carbonyl group and so are able to provide for compounds (V) containing a carboxylic acid functional group. Particular embodiments of Formula (V) such as compounds of Formula (Va) and (Vb) are thus able to be coupled to molecules of Formula (IV) using an amide coupling reaction to give compounds of Formula (BF-III).

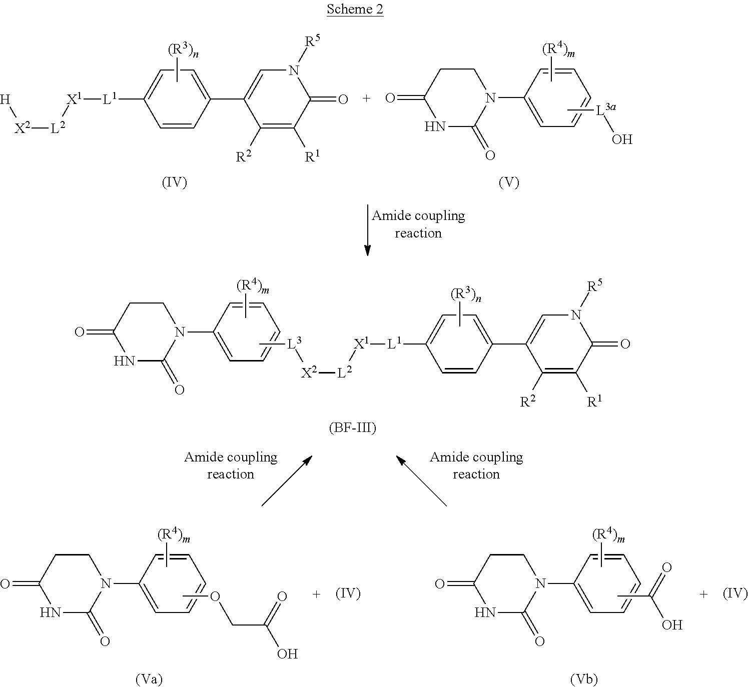

Compounds of Formula (BF-III) may also be synthesized according to Scheme 3. Compounds of Formula (V), such as (Va) and (Vb), may be transformed into a compound of Formula (VI) containing an activated ester where LG is defined as a leaving group; a specific example of (VI) is a pentafluorophenyl ester (VIa). Compounds of Formula (VI), including (VIa), may then be treated in a separate step with a compound of Formula (IV) in a solvent such as DMF and a base such as triethylamine to provide a compound of Formula (BF-III).

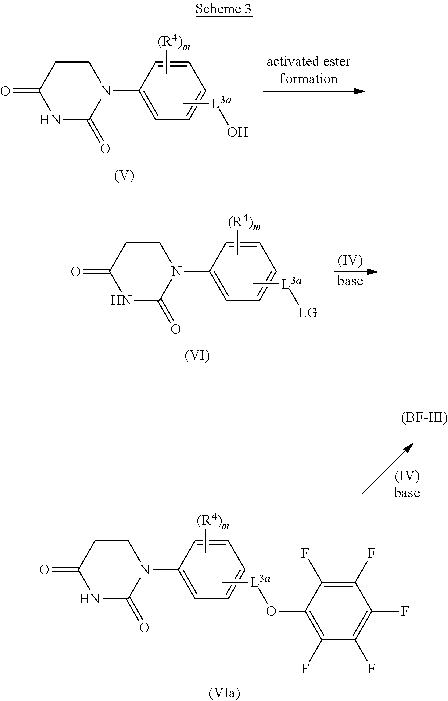

Compounds of Formula (III) may be synthesized in two steps from reaction between compounds of Formula (V) and Formula (VII) where PG represents a protecting group, such as t-butoxycarbonyl according to Scheme 4. Thus an amide coupling reaction using a reagent such as HATU, in a solvent such as DMF, in the presence of a base such as DIPEA, followed by a deprotection reaction using conditions such as TFA in DCM or HCl in 1,4-dioxane and methanol provides (III). Similarly, activated esters such as (VI), including compounds of Formula (VIa), may react with a compound of Formula (VII) in a solvent such as DMF and a base such as triethylamine to form an amide. Subsequent deprotection as described above provides a compound of Formula (III).

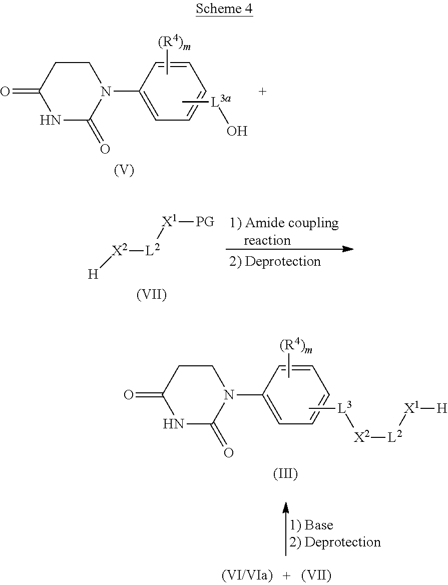

A two-step reductive amination then deprotection procedure may be applied to the synthesis of compounds of Formula (IV) starting from a compound of Formula (II) and a compound of Formula (VIIa), according to Scheme 5. In certain embodiments, where L² is symmetrical and X¹ and X² are the same, then (VII) and (VIIa) are equivalent.

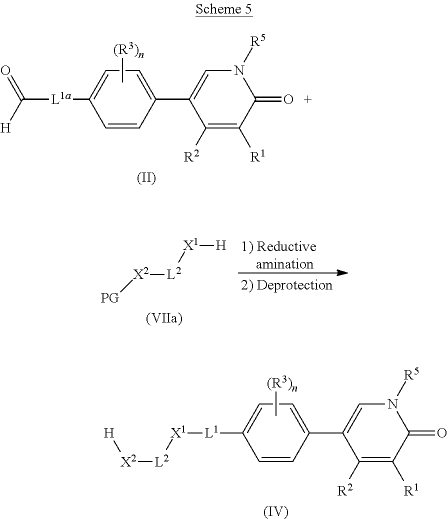

Compounds of Formula (BF-III) may be synthesized from a carboxylic acid of Formula (VIII) and a compound of Formula (III) according to Scheme 6, where L^{1b} is defined as the subset of linkers L¹, that contain a carbonyl group and so are able to provide for compounds (VIII) containing a carboxylic acid functional group. Suitable conditions include use of a reagent such as HATU, in a solvent such as DMF, in the presence of a base such as DIPEA. Furthermore, compounds of Formula (VIII) can be reacted with a compound of Formula (VIIa) under similar amide coupling conditions to provide after deprotection, using conditions such as TFA in DCM or HCl in 1,4-dioxane and methanol, a compound of Formula (IV).

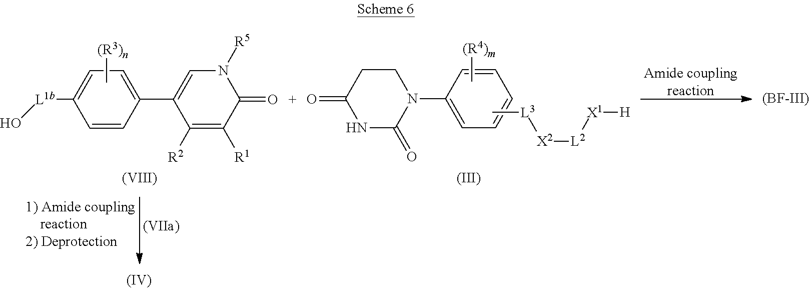

Compounds of Formula (IV) may be synthesized from a compound of Formula (IX) and a compound of Formula (X) according to Scheme 7. In this embodiment, L² is defined as a linker that is shorter by a single methylene group than L², where the Formula of L² allows (e.g. in an embodiment of (IV) where L² is —CH₂—, then L^{2a} is a bond). Thus, a more specific embodiment of compound (X) could be a compound of Formula (Xa). Compounds (X), including compounds such as (Xa) may react with (IX) using a reductive amination reaction. Conditions such as ZnCl₂ and NaBH₃CN, in a solvent mixture such as THF/DMSO and MeOH may be used. Subsequent deprotection reaction using conditions such as HCl in 1,4-dioxane and methanol or TFA in dichloromethane provides (IV).

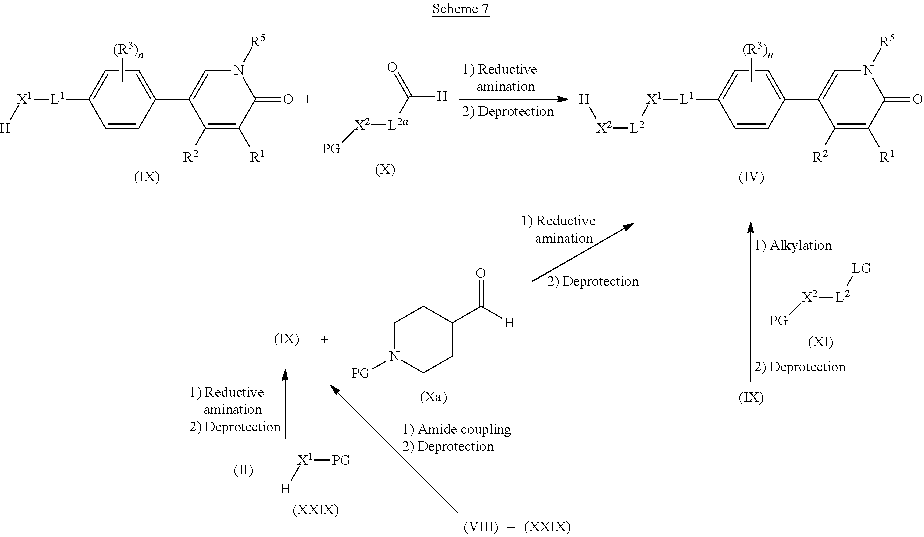

Compound (IX) may also react in an alkylation reaction with compounds of Formula (XI) and a base such a K₂CO₃ in a solvent such as acetonitrile, followed by a deprotection reaction as described above to furnish compounds of Formula (IV). Compounds of Formula (IX) can be synthesized by a reductive amination reaction of a compound of Formula (II) with a protected amine (XXIX) followed by deprotection, or alternatively using an amide coupling reaction between a compound of Formula (VIII) with a protected amine (XXIX) followed by deprotection. Conditions are analogous to those already described herein above for similar transformations.

Compounds of Formula (IX) may be formed from a palladium-catalyzed coupling reaction, such as a Suzuki reaction, between a compound of Formula (XII), where B(OR^{x})₂ defines either a boronic acid or ester (including cyclic boronic esters such as pinacol esters) and a compound of Formula (XIII), where Hal denotes a halogen, followed by a deprotection reaction (Scheme 8).

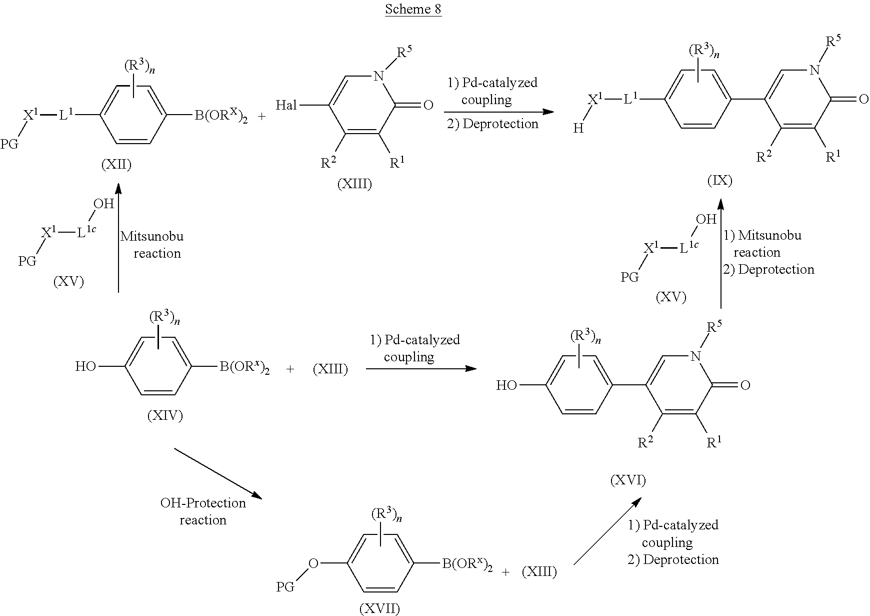

Conditions for the Suzuki reaction may include using a catalyst (e.g. PdCl₂(dppf)) and a base (e.g. Na₂CO₃) in a solvent mixture (e.g. dioxane/water). The subsequent deprotection reaction may use conditions such as HCl in 1,4-dioxane and dichloromethane or TFA in dichloromethane (Scheme 8).

Particular embodiments of (XII) where L¹ contains an oxygen atom may be synthesized from a compound of Formula (XIV) and a compound of Formula (XV) using a Mitsunobu reaction in the presence of a phosphine reagent such as triphenylphosphine and an azo carboxylate ester such as diethylazodicarboxylate. In this example, L^{1c} is defined as a linker that is shorter by a single oxygen atom than L¹, where the Formula of L¹ allows (e.g. in an embodiment of (XII) where L¹ is O, then L^{1c} is a bond; similarly where L¹ is —CH₂—O—, then L^{1c} is —CH₂—). A similar Mitsunobu procedure can be utilized to generate compounds of Formula (IX) directly from (XV) and a compound of Formula (XVI). Compound (XVI) can be generated from a palladium-catalyzed coupling of compounds of Formula (XIV) and (XIII) using conditions similar to those already described herein above. Optionally the hydroxyl of compound (XIV) can be protected with a protecting group such as a trialkylsilyl ether, using a trialkylsilyl halide or triflate reagent (such as TBDMSCl) and a base, such as imidazole in a solvent such as DCM, to produce a compound of Formula (XVII). These compounds (XVII) can then react with (XIII) using palladium-catalyzed coupling conditions such as those previously described, followed by a deprotection reaction using TBAF and TEA in a solvent such as THE to give (XVI) as depicted in Scheme 8.

Well known to those skilled in the art, reaction sequences can sometimes be performed in different orders, leading to similar compounds. For instance, Scheme 9 shows an alternative sequence for constructing compounds of Formula (BF-III) using similar procedures to those already described herein above. Thus, a compound of Formula (XII) can undergo a deprotection reaction to produce compound of Formula (XVIII).

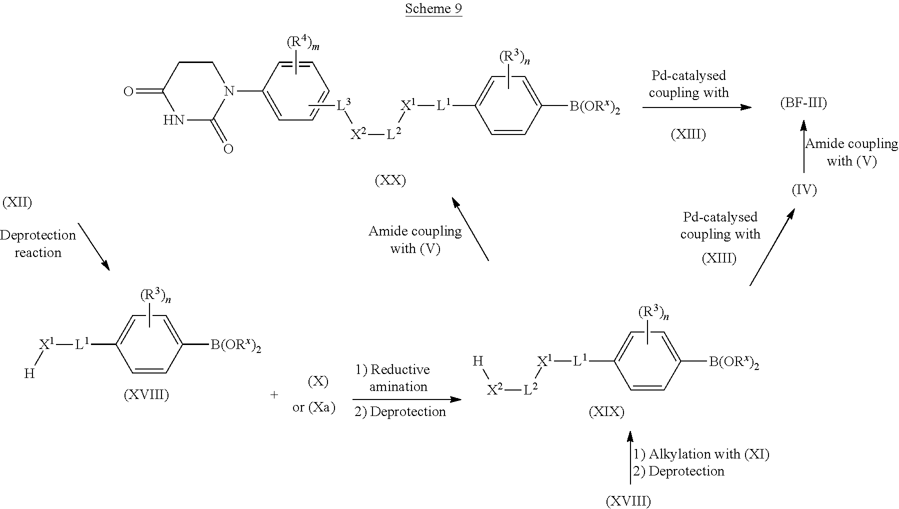

Conditions may include treatment with an acid such as TFA in a solvent such as dichloromethane. Compounds of Formula (XVIII) may then be converted to compounds of Formula (XIX) using a variety of routes shown in Scheme 9 (under similar conditions and by direct analogy to the synthesis of compounds (IV) from (IX) depicted in Scheme 7). Compounds (XIX) may then undergo an amide coupling reaction with a compound of Formula (V) using a reagent such as HATU, in a solvent such as DMF, in the presence of a base such as DIPEA to provide compounds of Formula (XX). These compounds (XX) can subsequently undergo a palladium-catalyzed coupling using a catalyst (e.g. PdCl₂(dppf)) and abase (e.g. Na₂CO₃) in a solvent mixture (e.g. dioxane/water) to provide compounds of Formula (BF-III). Compounds of Formula (XIX) may react directly in a palladium-catalyzed coupling under similar conditions to give a compound of Formula (IV). Formula (IV) can then react with (V) in an amide coupling as already described herein above.

Compounds of Formula (III) may also be synthesized according to Scheme 10. Thus, a compound of Formula (III) can be synthesized by reacting a compound of Formula (XXI) and a compound of Formula (XXII), where L^{2a} is as previously defined, in a reductive amination reaction, followed by a deprotection reaction using similar conditions to those already described herein above. Certain embodiments of (XXII) may be equivalent to certain embodiments of (X) if X¹ and X² are the same; an example being a compound of Formula (Xa), which can react in a similar sequence with (XXI) to give (III). Compounds of Formula (III) can also be accessed from compounds of Formula (XXI) in an alkylation with compounds (XXIII), followed by a subsequent deprotection reaction. It noteworthy that certain embodiments of (XXIII) may be equivalent to certain embodiments of (XI) if X¹ and X² are the same.

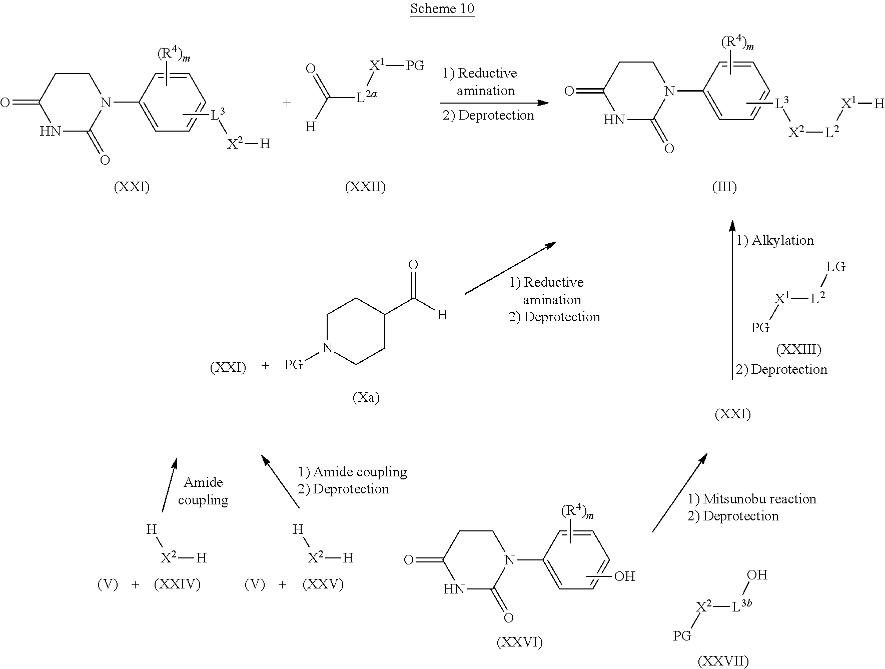

Compounds of Formula (XXI) may be made for example by reacting a compound of Formula (V) with a compound such as (XXIV) in an amide coupling reaction or with (XXV) in an amide coupling reaction followed by a deprotection reaction. An example of (XXIV) could be a symmetrical diamine, such as piperazine; an example of (XXV) could be a mono-protected diamine. For certain examples where L contains an oxygen atom, compounds of Formula (XXI) may be synthesized from a compound such as (XXVI) and a compound such as (XXVII), using a Mitsunobu reaction in the presence of a phosphine reagent such as triphenylphosphine and an azo carboxylate ester such as diethylazodicarboxylate, followed by a deprotection reaction, using conditions already described herein above. In this instance, L^{3b} is defined as a linker that is shorter by a single oxygen atom than L³, where the Formula of L³ allows (e.g. in an embodiment of (XXI) where L³ is O, then L^{3b} is a bond; similarly where L³ is —CH₂—O—, then L^{3b} is —CH₂—).

Compounds of Formula (III) can be synthesized according to Scheme 11. Thus, an intermediate of Formula (XXVIII) can undergo reductive amination with a compound of Formula (XXIX), then the product deprotected under conditions previously described to produce (III). Similarly, an alkylation, deprotection sequence starting from (XXX) and (XXIX) leads to compounds of Formula (III). Again, similar reaction conditions to those already described herein above can apply.

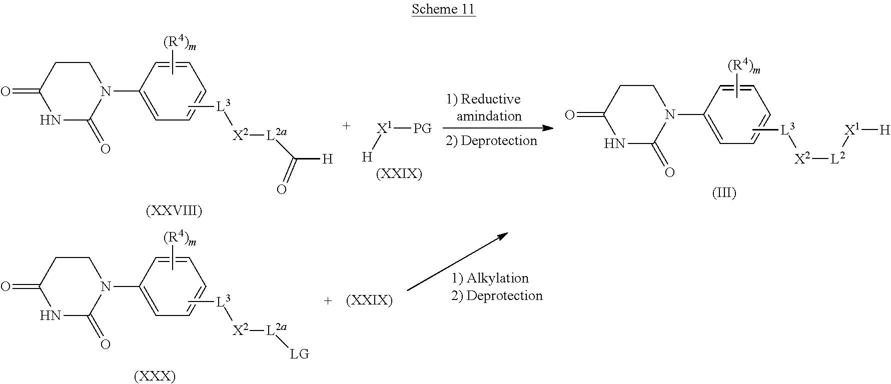

Compounds of Formula (BF-III) may be made by analogy, according to Scheme 12, using a compound of Formula (IX). A reductive amination of (IX) with (XXVIII) gives a compound of Formula (BF-III). An alkylation of (IX) with (XXX) can also directly provide a compound of Formula (BF-III).

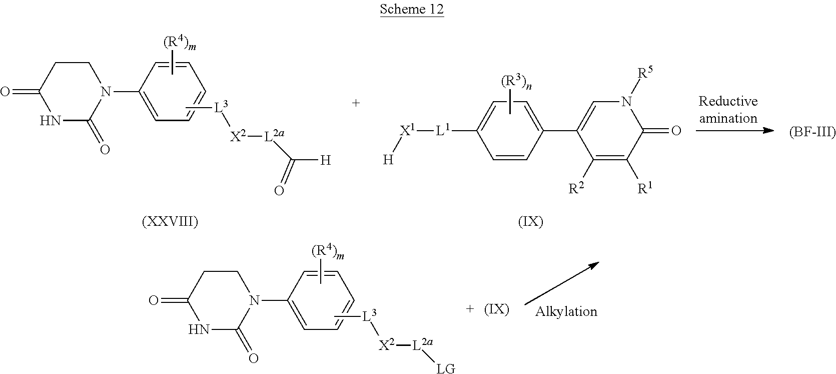

Compounds of Formula (BF-III), wherein X¹ contains an amine functionality such as a nitrogen-containing heterocyclyl, e.g., a piperidinyl or piperazinyl, may be made according to Scheme 12a. Reductive amination of a compound of formula (IV) and an aldehyde of formula (XXXXI) using conditions such as ZnCl₂ and NaBH₃CN, in a solvent mixture such as THE and MeOH, provides a compound of Formula (BF-III). L³ is defined as a linker that is shorter by a single methylene group than L³, where the formula of L³ allows (e.g. in an embodiment where L is —OCH₂CH₂—, then L^{3c} is —OCH₂—). Suitable L³ include C₁₋₆ alkylene and C₁₋₆ heteroalkylene.

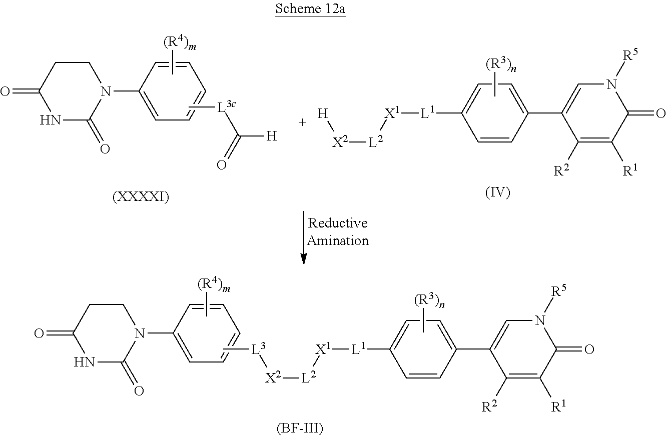

Aldehydes of formula (XXXXI) can also undergo a reductive amination reaction, using similar conditions to those described above, with a compound of formula (VII) followed by a deprotection reaction such as under acidic conditions to produce compounds of formula (III) as described in Scheme 12b. Compounds of formula (XXXXI) may be prepared by an oxidative cleavage reaction of a corresponding alkene (XXXXII), for example by ozonolysis in a solvent such as DCM or by oxidation of an alcohol of formula (XXXXIII) using an oxidant such as Dess-Martin periodinane.

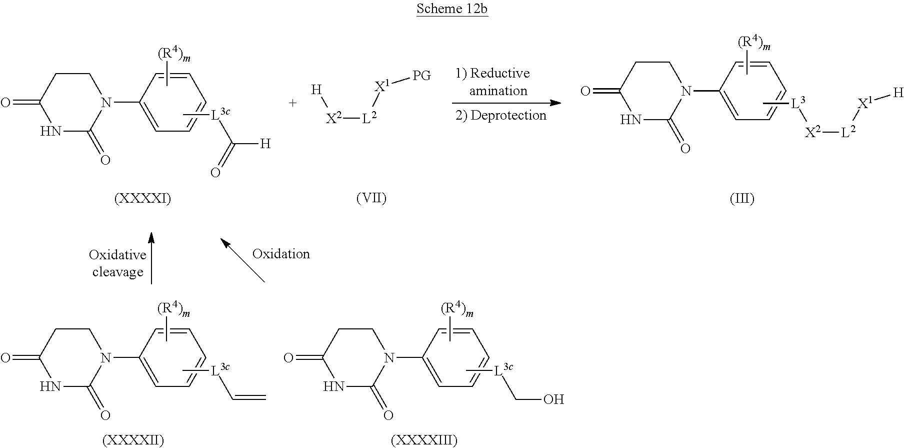

Alkylation of a compound of formula (III) with an alkyl halide of formula (XXXXIV), such as 1-bromo-4-bromomethyl benzene, in a solvent such as acetonitrile with a base such as DIPEA may provide compounds of formula (XXXXV). These molecules of formula (XXXXV) may react with a suitable boronic acid or boronic ester derivative of formula (XXXXVI) in a palladium catalyzed cross coupling reaction to provide compounds of formula (BF-III) according to Scheme 12c. Suitable conditions include using a catalyst such as PdCl₂(dppf)-CH₂Cl₂ and a base such as sodium carbonate in dioxane and water in a microwave reactor at ca. 100° C., for 65 minutes. Compounds of formula (XXXXV) may also be produced by reductive amination of (III) with aldehydes of formula (XXXII) under similar conditions to those described previously. The boronic acid or its derivative (XXXXVI) can be synthesized using a palladium-catalyzed halogen boron exchange reaction starting from the corresponding halogenated compound (XIII). Suitable conditions include reaction with BISPIN using a catalyst such as PdCl₂(dppf)-CH₂Cl₂ and a base such as potassium acetate in dioxane at ca. 90° C., for 3 h.

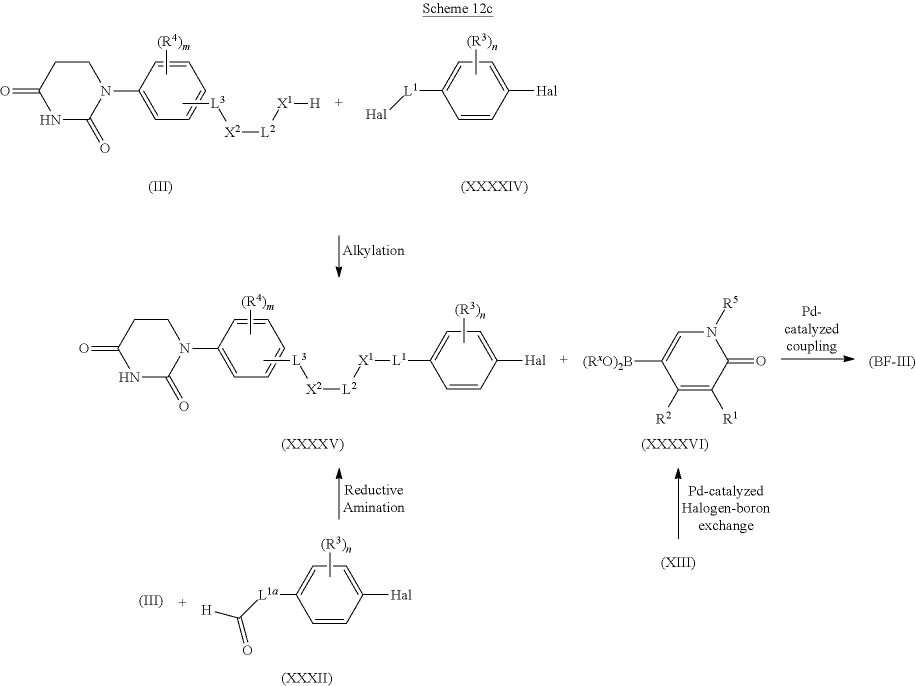

Compounds of Formula (II) can be synthesized according to Scheme 13 from a palladium-catalyzed coupling between compounds of Formula (XXXI) and (XIII). Hal is defined as a halogen atom, such as a bromine or iodine atom and LG refers to a leaving group such as a halogen group or mesylate. Compounds (XXXI), where B(OR^{x})₂ defines either a boronic acid or ester (including cyclic boronic esters such as pinacol esters), can be synthesized from the corresponding aryl halide (XXXII) by halogen boron exchange. Example conditions are to use a boronic ester dimer (eg bis(pinacolato)diboron), a Pd catalyst such as PdCl₂(dppf) and a base such as KOAc in a solvent such as 1,4-dioxane or DME. Compounds (XIII) can be formed by alkylation of a compound of Formula (XXXIII) using a base and an alkylating agent R⁵-LG such as an alkyl halide. In an embodiment, R⁵ is methyl. In an embodiment, R⁵ is C₂₋₆ alkyl. In an embodiment, R⁵ is butyl. Compounds of Formula (II) may also derive from the corresponding alcohol of Formula (XXXIV) by oxidation using a reagent such as 2-iodoxybenzoic acid (IBX) in DMSO.

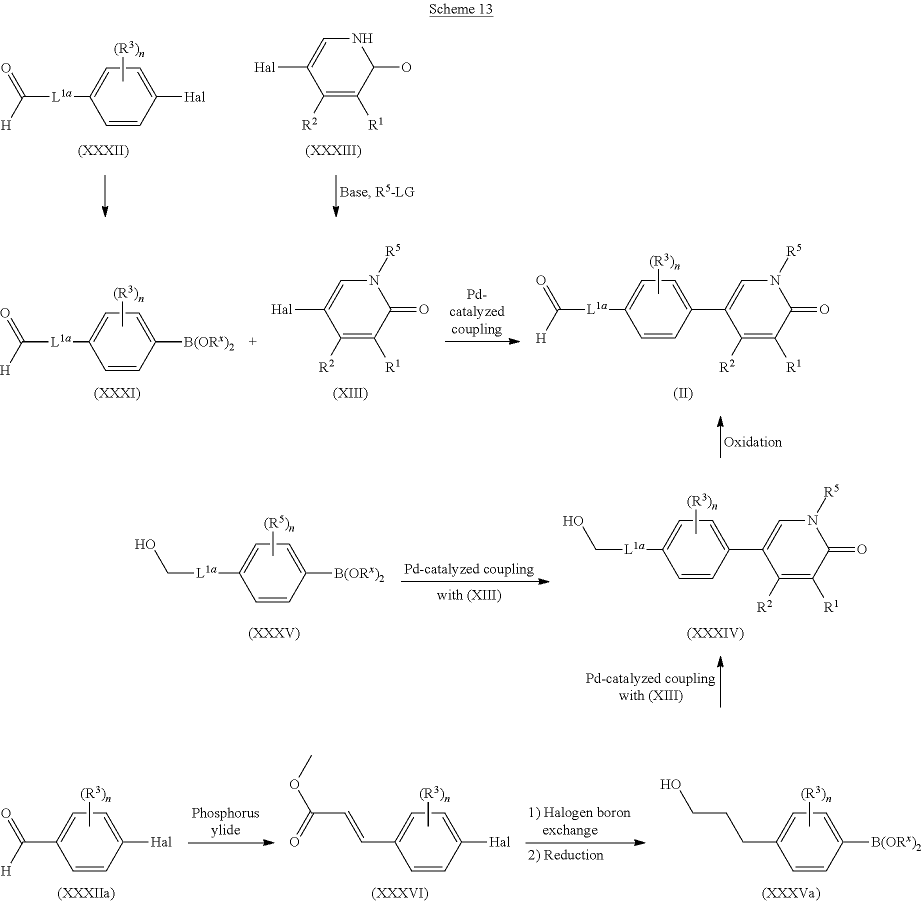

In turn, compounds of Formula (XXXIV) may be produced by a Pd-catalyzed reaction between (XIII) and (XXXV). A particular embodiment of Formula (XXXV), being (XXXVa), can be produced by treating aldehydes of Formula (XXXIIa) with a phosphorus ylide reagent produced in a reaction such as a Homer-Emmons reaction, using for example methyl 2-(dimethoxyphosphoryl)acetate and a base, such as NaH, in THF. This provides compounds of Formula (XXXVI) which can undergo halogen-boron exchange using conditions already described above, followed by reduction to give compounds of Formula (XXXVa). The reduction may take place sequentially, by reducing the double bond with hydrogen gas and a Pd/C catalyst in a solvent such as methanol, followed by reduction of the ester functionality with a reductant such as LiAlH₄ in a solvent such as THF.

According to Scheme 14, compounds of Formula (XXXII) and (VIIa) may provide a compound of Formula (XXXVII) under reductive amination conditions already described herein above. A halogen-boron exchange reaction can then lead to compounds of Formula (XXXVIII), which may then undergo a palladium-catalyzed coupling followed by a deprotection reaction, eg using an acid such as HCl in solvents such as DCM, MeOH and dioxane to provide (IV). Simple deprotection of (XXXVIII) provides compounds of Formula (XIX).

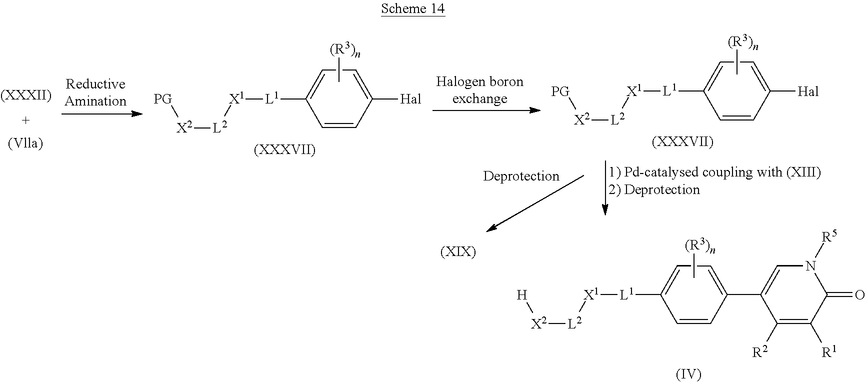

Compounds of Formula (V) may be synthesized according to Scheme 15 from the corresponding amines (XXXIX), through conjugate addition to acrylic acid usually by heating above 70° C. with a co-solvent such as water, followed by reaction with urea and acetic acid, also at elevated temperature such as 120° C., to form the dihydrouracil. This reaction works effectively for the preparation of subclasses of (V), such as (Va) and (Vb) from starting materials such as (XXXIXa) and (XXXIXb) respectively. In the case of (Va), the dihydrouracil formation may be carried out on the corresponding phenolic acetate ester (XXXIXa) and the ester can be hydrolysed using acidic conditions, such as HCl treatment in a final step. Compounds such as (XXXIXa) are available from aminophenols with a protected nitrogen (XXXX), for example a Boc-protected nitrogen, in two steps. First, alkylation of the phenol using a base such as Cs₂CO₃ and a 2-haloacetate ester, such as methyl bromoacetate, in a solvent such as acetone with an additive such as potassium iodide provides an intermediate that can undergo N-deprotection using for example an acid such as TFA in a solvent such as dioxane to provide compounds of Formula (XXXIXa).

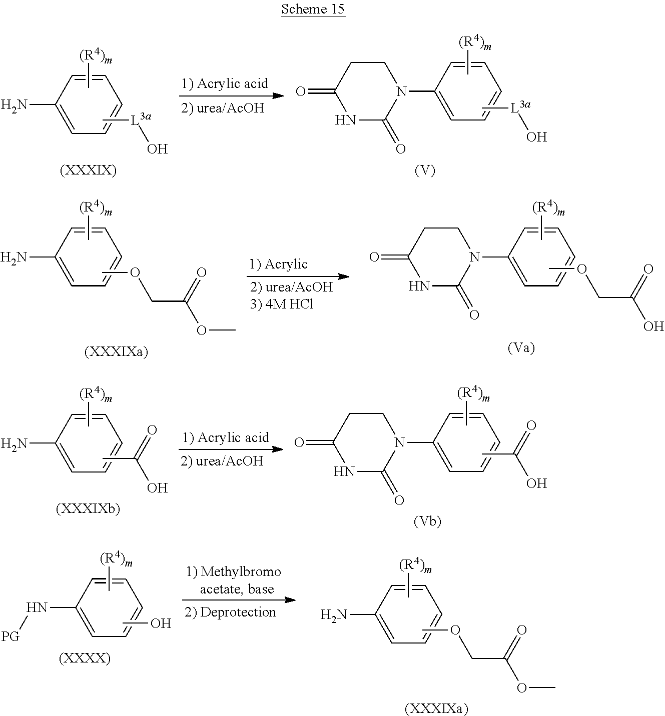

Compounds of Formula (BF-IV′), wherein X is a nitrogen-containing heterocyclyl, e.g., a piperidinyl or piperazinyl and R¹, R², R³, R^{4′}, X², L¹, L², L³ and n are as previously defined, may be made according to Scheme 16. Thus, reaction of a compound of Formula (II′) and a compound of Formula (III′), for example in a reductive amination reaction, using conditions such as ZnCl₂ and NaBH₃CN, in a solvent mixture such as THF/DMSO and MeOH, provides a compound of Formula (BF-IV′). L^{1a} is defined as a linker that is shorter by a single methylene group than L¹, where the formula of L¹ allows (e.g. in an embodiment where L¹ is —CH₂CH₂—, then L^{1a} is —CH₂—). Suitable L¹ include C₁₋₆ alkylene and C₁₋₆ heteroalkylene.

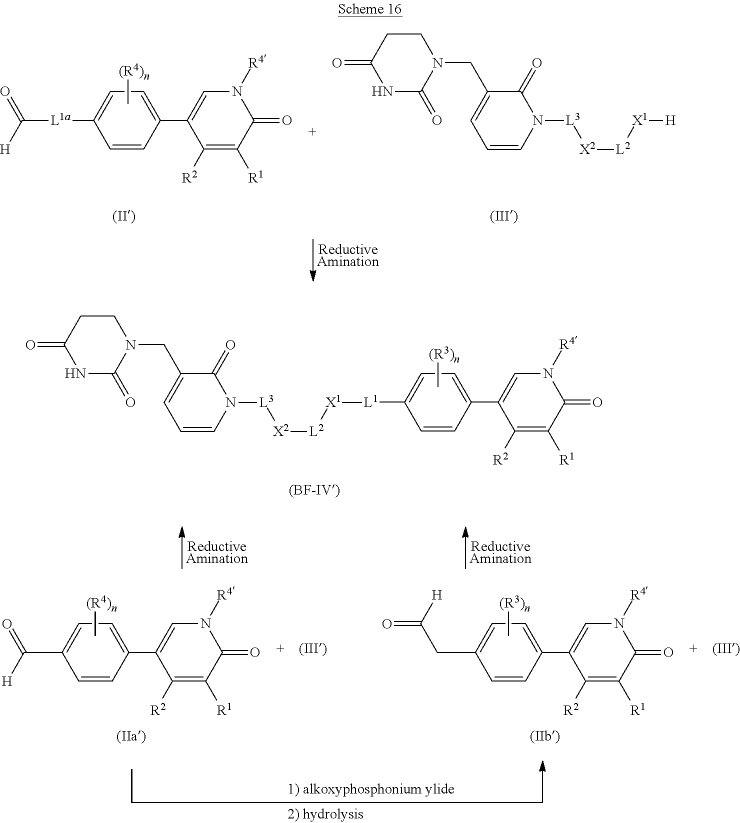

In an embodiment, specific examples of compounds of Formula (II′) may include (IIa′) and (IIb′); both (IIa′) and (IIb′) can react similarly with compounds of Formula (III′) (e.g., in a reductive amination reaction) to provide a compound of Formula (BF-IV′). In an embodiment, compounds of Formula (II′) with longer chain extension can be made, for example, by conversion of (IIa′) into (IIb′). This can be achieved, for example, by reacting (IIa′) with an alkoxyphosphonium ylide, such as that derived from (methoxymethyl)triphenylphosphonium halide and a base (e.g., potassium tert-butoxide (KO^{t}Bu), etc.), in a solvent (e.g., tetrahydrofuran (THF), diethyl ether (Et₂O), etc.) to produce an enol ether which is then hydrolyzed in a second step.

In an embodiment, compounds of Formula (BF-IVa′), wherein X¹ is piperidinyl, L¹ is C₁₋₆ alkylene, and R¹, R², R³, R^{4′}, X², L², L³, and n are as previously defined, may be made according to Scheme 16a. For example, reductive amination of a compound of Formula (II′) and a compound of Formula (IIIa′) using conditions such as ZnCl₂ and NaBH₃CN, in a solvent mixture, such as THF/DMSO and MeOH, provides a compound of Formula (BF-IVa′).

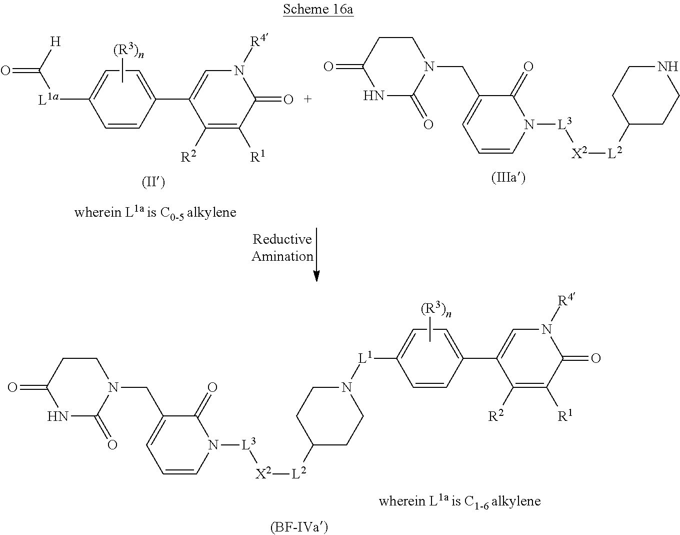

Alternatively, compounds of Formula (BF-IV′) may be synthesized from a compound of Formula (IV′), wherein X² is a nitrogen-containing heterocyclyl, e.g., a piperidinyl or a piperazinyl and a compound of Formula (V′) according to Scheme 17, via a reductive amination reaction, using conditions such as ZnCl₂ and NaBH₃CN, in a solvent mixture such as THF/DMSO and MeOH. L^{3c} is defined as a linker that is shorter by a single methylene group than L³, where the formula of L³ allows (e.g. in an embodiment where L³ is —CH₂CH₂—, then L^{3c} is —CH₂—). Suitable L³ include C₂₋₆ alkylene and C₂₋₆ heteroalkylene.

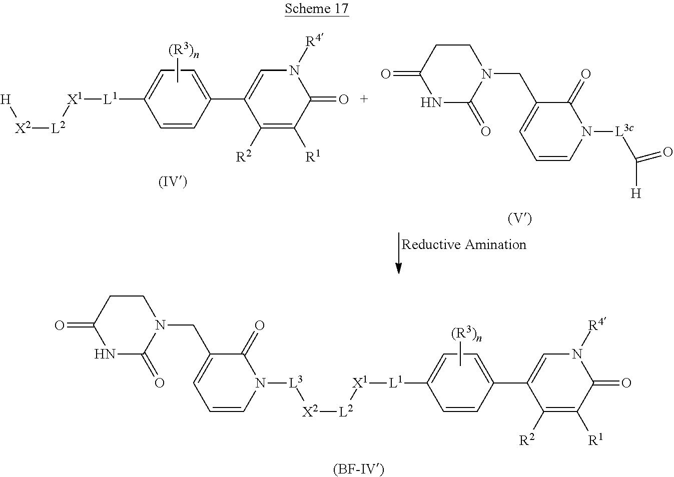

Compounds of Formula (III′) and Formula (IV′) can be synthesized as shown in Scheme 18. For example, compounds of Formula (III′) may be synthesized in two steps from compounds of Formula (V′) and Formula (VI′) where PG represents a protecting group, such as tert-butoxycarbonyl according to Scheme 18. Reductive amination under conditions such as ZnCl₂ treatment, followed by sodium cyanoborohydride in solvents such as THF, DMSO, and MeOH, followed by deprotection under acidic conditions, e.g., using HCl in 1,4-dioxane and dichloromethane, provides the compound of Formula (III′). Similarly, a two-step reductive amination then deprotection procedure may be applied to the synthesis of compounds of Formula (IV′) starting from a compound of Formula (II′) and a compound of Formula (VIa′). In certain embodiments, when L² is symmetrical and X¹ and X² are the same, then (VI′) and (VIa′) are equivalent.

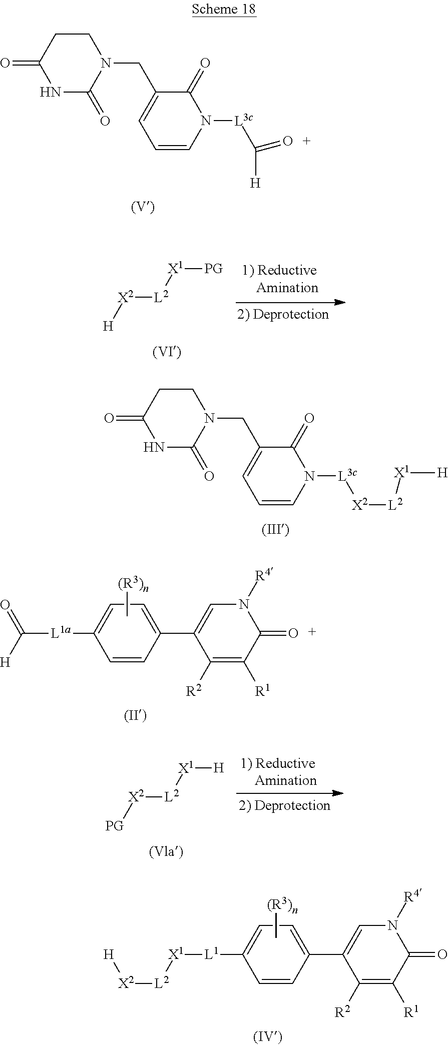

Compounds of Formula (II′) can be produced via a palladium-catalyzed coupling reaction, such as a Suzuki reaction, of a compound of Formula (VII′) and a compound of Formula (VIII′) using a suitable catalyst (e.g., PdCl₂(dppf), etc.) and a base (e.g., Na₂CO₃, etc.) in a solvent mixture (e.g., dioxane/water, etc.), according to Scheme 18.

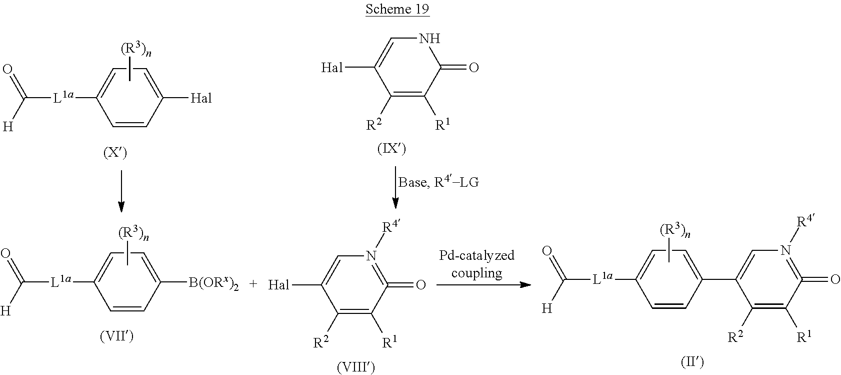

In Scheme 19, Hal is a halogen atom, such as a bromine or iodine atom, and LG is a leaving group, such as a halogen group or mesylate. Compounds of Formula (VII′), where B(OR)₂ defines either a boronic acid or ester (including cyclic boronic esters such as pinacol esters), can be synthesized from the corresponding aryl halide, compound (X′) by halogen boron exchange. Example conditions include, a boronic ester dimer (e.g., bis(pinacolato)diboron), a Pd catalyst such as PdCl₂(dppf) and a base such as KOAc in a solvent such as 1,4-dioxane. Compounds of Formula (VIII′) can be synthesized by alkylation of a compound of Formula (IX′) using a base and an alkylating agent R^{4′}-LG (e.g., an alkyl halide). In an embodiment, R^{4′} is methyl. In an embodiment, R^{4′} is C₂₋₆ alkyl. In an embodiment, R^{4′} is butyl.

As is well-known to those skilled in the art, reaction sequences can sometimes be performed in different orders, leading to similar compounds. For instance, Scheme 20 shows an alternative sequence for constructing compounds of Formula (IV′) using similar procedures to those already described herein above. Thus, compounds of Formula (X′) and (VIa′) may provide a compound of Formula (XI′) under reactive amination conditions as previously described herein above. A halogen-boron exchange reaction leads to compounds of Formula (XII′), which may then undergo a palladium-catalyzed coupling followed by a deprotection reaction, e.g., using an acid such as HCl in solvents such as DCM, MeOH, or dioxane to provide a compound of Formula (IV′).

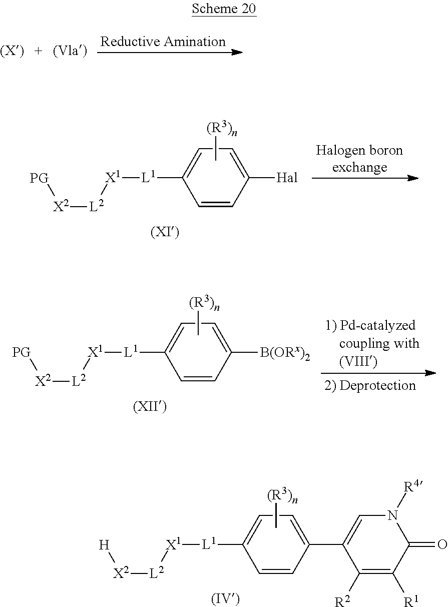

A compound of Formula (V′) may be derived from a compound of Formula (XIII′) using an oxidative cleavage reaction, such as an ozonolysis, as shown in Scheme 21. Compounds of Formula (XIII′) may be derived from the corresponding amine of Formula (XIV′) through conjugate addition of the amine to acrylic acid, followed by reaction with urea and acetic acid to form the dihydrouracil of Formula (XIII′). Amines of Formula (XIV′) may be derived from 3-cyanopyridin-2-one by first reducing, the nitrile using conditions such as hydrogenation in the presence of Raney-Nickel in methanol/ammonia solution, then protecting the nitrogen to provide the compound of Formula (XV′), for example, with a typical amine protecting group such as a tert-butoxycarbonyl group. Alkylation of Intermediate (XV′) with an alkylating agent such as allyl bromide and a base such as potassium carbonate in a solvent such as DMF followed deprotection using, for example, HCl in a solvent mixture of DCM and dioxane provides the compound of Formula (XIV′). Alternatively, compounds of Formula (V′) may be synthesized from a compound of Formula (XV′) through alkylation using an alkylating agent containing a protected alcohol followed by removal of the protecting group (PG) to provide a molecule with Formula (XVI′). Dihydrouracil formation using the method previously described provides compounds of Formula (XVII′). Alcohol deprotection followed by oxidation to the aldehyde using an oxidant such as Dess-Martin periodinane optionally in a solvent provides the compound of Formula (V′).

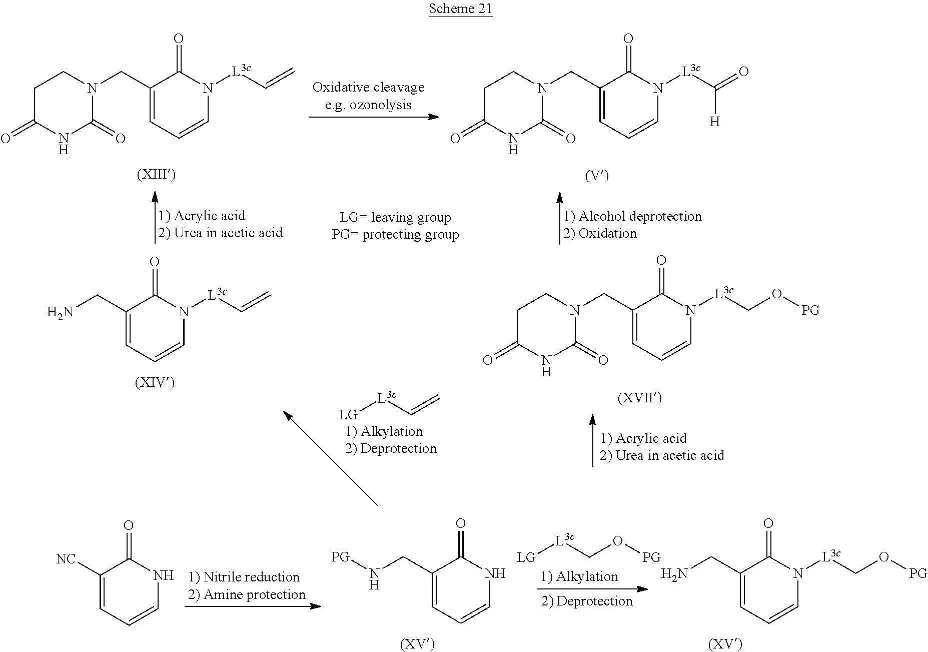

A mixture of enantiomers, diastereomers, and cis/trans isomers resulting from the process described above can be separated into their single components by chiral salt technique, chromatography using normal phase, reverse phase or chiral column, depending on the nature of the separation.

Any resulting racemates of compounds of the present disclosure or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present disclosure into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid, or camphor-10-sulfonic acid. Racemic compounds of the present disclosure or racemic intermediates can also be resolved by chiral chromatography, e.g., high pressure liquid chromatography (HPLC) using a chiral adsorbent.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

It should be understood that in the description and formula shown above, the various groups R¹, R², R³, R⁴, R^{4′}, R⁵, X¹, X², L¹, L², L³, m, n and other variables are as defined above, except where otherwise indicated. Furthermore, for synthetic purposes, the compounds of Schemes 1, 1a, 2-12, 12a, 12b, 12c, 13-16, 16a, and 17-21 are merely representative with elected radicals to illustrate the general synthetic methodology of the compounds disclosed herein. The preparation of specific intermediates and examples using the general methods described above is provided in detail in the experimental section.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof, which may suggest themselves to those, skilled in the art without departing from the spirit of the present disclosure and/or scope of the appended claims.

Compounds of the disclosure may be prepared by methods known in the art of organic synthesis. In all of the methods it is understood that protecting groups for sensitive or reactive groups may be employed where necessary in accordance with general principles of chemistry. Protecting groups are manipulated according to standard methods of organic synthesis (T.W. Green and P.G.M. Wuts (1999) Protective Groups in Organic Synthesis, 3rd edition, John Wiley & Sons). These groups are removed at a convenient stage of the compound synthesis using methods that are readily apparent to those skilled in the art.

Temperatures are given in degree Celsius. Abbreviations used are those conventional in the art and listed below.

All starting materials, building blocks, reagents, acids, bases, dehydrating agents, solvents, and catalysts utilized to synthesise the compounds of the present invention are either commercially available or can be produced by organic synthesis methods known to one of ordinary skill in the art. Further, the compounds of the present invention can be produced by organic synthesis methods known to one of ordinary skill in the art as shown in the following examples.

### Abbreviations

Å Angstrom
ACN acetonitrile
aq. aqueous
bar bar
BISPIN bis(pinacolato)diboron
Boc tert-butyloxycarbonyl
br broad
CHX cyclohexane
conc. concentrated
Cs₂CO₃ cesium carbonate
d doublet
dd doublet of doublets
DCM dichloromethane
DEAD diethyl azodicarboxylate
DIAD diisopropyl azodicarboxylate
DIEA diethylisopropylamine
DIPEA diisopropylethylamine
DME 1,2-dimethoxyethane
DMF N,N-dimethylformamide
DMSO dimethylsulfoxide
dppf 1,1′-bis(diphenylphosphino)ferrocene
Et₂O diethylether
EtOAc ethyl acetate
EtOH ethanol
h hour(s)
H₂ hydrogen
H₂SO₄ sulfuric acid
H₃PO₄ phosphoric acid
HATU 1-[bis(dimethylamino)methylene]-1,2,3-triazolo[4,5-b]pyridinuim 3-oxide hexafluorophosphate
HCl hydrogen
HCOOH formic acid
HEK human embryonic kidney
HOAc acetic acid
HPLC high performance liquid chromatography
IBX 2-iodoxybenzoic acid
iPrOH isopropanol
K₂CO₃ potassium carbonate
KI potassium iodide
KOAc potassium acetate
1/L liter
LC-MS liquid chromatography and mass spectrometry
LiBr lithium bromide
LiHMDS lithium bis(trimethylsilyl)amide
m multiplet
M molar
MeOH methanol
mg milligram
MgSO₄ magnesium sulfate
MHz megahertz
min minute(s)
ml/mL milliliter
mm millimeter
mM millimolar
μm micrometer
μM micromolar
μmol micromoles
μl microliter
mol moles
mmol millimole(s)
mM millimolar
MS mass spectrometry
MsCl methanesulfonyl chloride
MTBE methyl tert-butyl ether
MW molecular weight
NaBH₃CN sodium cyanoborohydride
NaBH(OAc)₃ sodium triacetoxyborohydride
Na₂CO₃ sodium carbonate
NaH sodium hydride
NaHCO₃ sodium bicarbonate
NaNO₂ sodium nitrite
NaOAc sodium acetate
NaOH sodium hydroxide
NH₄Cl ammonium chloride
NH₄HCO₃ ammonium hydrogen carbonate
NH₄OAc ammonium acetate
NH₄OH ammonium hydroxide
nm nanometer
NMR nuclear magnetic resonance
OsO₄ osmium tetroxide
Pd/C palladium on activated charcoal
PdCl₂(dppf) 1,1′-Bis(diphenylphosphino)ferrocene-palladium(II)dichloride
PdCl₂(dppf)-CH₂Cl₂ 1,1′-Bis(diphenylphosphino)ferrocene-palladium(II)dichloride
dichloromethane complex
PE petroleum ether
PPh₃ triphenylphosphine
ppm parts per million
PtO₂ platinum oxide
RM reaction mixture
Rt retention time
RT room temperature
s singlet
sat. saturated
SCX strong cation exchange
SFC supercritical fluid chromatography
t triplet
t-BuOK potassium tert-butoxide
TEA triethylamine
TFA trifluoroacetic acid
THE tetrahydrofuran
ZnCl₂ zinc chloride

Analytical Methods:

General Conditions:

NMR: NMR spectra were acquired on Bruker AVANCE 400 MHz, 500 MHz or 600 MHz NMR spectrometers using ICON-NMR, under TopSpin program control. Spectra were measured at 25° C., unless indicated otherwise, and were referenced relative to the solvent resonance.

LC-MS: Mass spectra were acquired on LC-MS systems using electrospray, chemical or electron impact ionization methods from a range of instruments of the following configurations:
Waters Acquity UPLC/SQD system, using a photodiode array detector and a single quadrupole mass detector
Agilent 1200 systems with G 6110 series mass detector
Agilent 1290 Infinity II with DAD (photodiode array detector) and single quadrupole mass detector with ESI and APCI ionization (multi-mode).
Waters AcQuity UPLC with PDA (photodiode array detector), ELSD (evaporative light scattering detector) and single quadrupole mass detector with ESI ionization
Waters AutoPurification System with PDA (photodiode array detector) and single quadrupole mass detector with ESI ionization.
   [M+H]⁺ refers to protonated molecular ion of the chemical species.
   [M−H]⁻ refers to molecular ion of the chemical species with loss of one proton.
   [M+Na]⁺ refers to molecular ion of the chemical species with addition of one sodium ion.
   [M-Boc+H]⁺ refers to protonated molecular ion of the chemical species without a Boc protecting group.

Method A or Method LCMS_A031:
Column: SUNFRE C18 (4.6×50 mm, 3.5 μm)
Column temperature: 50° C.
Eluents: A: aq. TFA (0.01%)
   B: ACN containing TFA (0.01%)
Flow rate: 2.0 ml/min
Gradient: from 5% to 95% B in 1.2 min

Method B:
Column: SunFire C18 (4.6×50 mm, 3.5 μm)
Column temperature: 50° C.
Eluents: A: aq. TFA (0.01%)
   B: ACN containing TFA (0.01%)
Flow rate: 2.0 ml/min
Gradient: from 5% to 95% B in 1.4 min

Method C:
Column: XBridge C18 (4.6×50 mm, 3.5 μm)
Column temperature: 50° C.
Eluents: A: aq. NH₄HCO₃ (10 mM)
   B: ACN
Flow rate: 1.8 ml/min
Gradient: from 5% to 95% B in 1.5 min

Method D or Method LCMS_PL1:
Column: ACQUITY UPLC® HSS T3 (2.1×50 mm, 1.8 m)
Column temperature: 60° C.
Eluents: A: aq. HCOOH (0.05%) containing NH₄OAc (3.75 mM)
   B: ACN containing HCOOH (0.04%)
Flow rate: 1.0 ml/min
Gradient: from 5% to 98% B in 1.4 min

Method E:
Column: SunFire C18 (4.6×50 mm, 3.5 μm)
Column temperature: 50° C.
Eluents: A: aq. TFA (0.01%)
   B: ACN containing TFA (0.01%)
Flow rate: 2.0 ml/min
Gradient: from 5% to 95% B in 1.2 min, followed by 95% B for 1.3 min

Method F or Method LCMSA010:
Column: XBridge C18 (4.6×50 mm, 3.5 μm)
Column temperature: 50° C.
Eluents: A: aq. NH₄HCO₃ (10 mM)
   B: ACN
Flow rate: 1.8 ml/min
Gradient: from 5% to 95% B in 1.5 min, followed by 95% B for 1.5 min

Method G or Method LCMSA039:
Column: XBridge C18 (4.6×50 mm, 3.5 μm)
Column temperature: 40° C.
Eluents: A: aq. NH₄HCO₃ (10 mM)
   B: ACN
Flow rate: 1.8 ml/min
Gradient: from 5% to 95% B in 1.4 min, followed by 95% B for 1.6 min

Method H or Method LCMSA043:
Column: XBridge C18 (4.6×50 mm, 3.5 μm)
Column temperature: 40° C.
Eluents: A: aq. NH₄HCO₃ (10 mM)
   B: ACN
Flow rate: 2.0 ml/min
Gradient: from 5% to 95% B in 1.5 min

Method I:
Column: SunFire C18 (3×30 mm, 2.5 μm)
Column temperature: 50° C.
Eluents: A: aq. TFA (0.01%)
   B: ACN containing TFA (0.01%)
Flow rate: 1.5 ml/min
Gradient: from 5% to 95% B in 1.5 min

Method J:
Column: Phenomenex C18 (3.0×30 mm, 5 μm)
Column temperature: 50° C.
Eluents: A: aq. NH₄HCO₃ (10 mM)
   B: ACN
Flow rate: 1.5 ml/min
Gradient: from 5% to 95% B in 1.5 min and 95% B for 0.7 min

Method K:
Column: ACQUITY UPLC® HSS T3 (2.1×50 mm, 1.8 m)
Column temperature: 60° C.
Eluents: A: aq. HCOOH (0.05%) containing NH₄OAc (3.75 mM)
   B: ACN containing HCOOH (0.04%)
Flow rate: 1.0 ml/min
Gradient: from 1% to 98% B in 1.4 min

Method L:
Column: ACQUITY UPLC® HSS T3 (2.1×100 mm, 1.8 m)
Column temperature: 60° C.
Eluents: A: aq. HCOOH (0.05%) containing NH₄OAc (3.75 mM)
   B: ACN containing HCOOH (0.04%)
Flow rate: 0.8 ml/min
Gradient: from 5% to 98% B in 9.4 min

Method M:
Column: ACQUITY UPLC® BEH C18 (2.1×50 mm, 1.7 m)
Column temperature: 80° C.
Eluents: A: aq. HCOOH (0.05%) containing NH₄OAc (3.75 mM)
   B: iPrOH containing HCOOH (0.05%)
Flow rate: 0.6 ml/min
Gradient: from 5% to 98% B in 1.7 min

Method N:
Column: ACQUITY UPLC® BEH C18 (2.1×100 mm, 1.7 m)
Column temperature: 80° C.
Eluents: A: aq. HCOOH (0.05%) containing NH₄OAc (3.75 mM)
   B: iPrOH containing HCOOH (0.05%)
Flow rate: 0.4 ml/min
Gradient: from 5% to 60% B in 8.4 min and from 60% to 98% B in 1 min

Method O:
Column: SunFire C18 (4.6×50 mm, 3.5 μm)
Column temperature: 50° C.
Eluents: A: aq. TFA (0.01%)
   B: ACN containing TFA (0.01%)
Flow rate: 2.0 mL/min
Gradient: 5% to 95% B in 1.3 min

Method P:
Column: HALO C18 (4.6×30 mm, 2.7 μm)
Column temperature: 50° C.
Eluents: A: aq. TFA (0.01%)
   B: ACN containing TFA (0.01%)
Flow rate: 2.2 mL/min
Gradient: 5% to 95% B in 1.0 min

Method LCMSA022:
Column: SunFire C18 (3×30 mm, 2.5 μm)
Column temperature: 50° C.
Eluents: A: aq. TFA (0.01%)
   B: ACN containing TFA (0.01%)
Flow rate: 1.5 ml/min
Gradient: 5% to 95% B in 1.5 min

Method LCMSA027:
Column: Chromolith fast gradient RP-18e (50×3 mm)
Column temperature: 35° C.
Eluents: A: aq. TFA (0.01%)
   B: ACN containing TFA (0.01%)
Flow rate: 1.5 ml/min
Gradient: 5% to 100% B in 0.8 min, followed by 100% B for 1.0 min

Method LCMSA042:
Column: Phenomenex C18 (3.0×30 mm, 5 μm)
Column temperature: 50° C.
Eluents: A: aq. NH₄HCO₃ (10 mM)
   B: ACN
Flow rate: 1.5 ml/min
Gradient: 5% to 95% B in 1.5 min, followed by 95% B for 0.7 min

Method LCMS-ACO-ODA #KAB0746—Basic:
Column: ACQUITY UPLC® BEH C18 (2.1×30 mm, 1.7 μm)
Column temperature: 50° C.
Eluents: A: Water+NH₄OH (5 mM)
   B: ACN+NH₄OH (5 mM)
Flow rate: 1.0 ml/min
Gradient: 2% to 98% B in 1.4 min

Method LCMS-ACO-ODA #KAB0746—Acidic:
Column: ACQUITY UPLC® BEH C18 (2.1×30 mm, 1.7 μm)
Column temperature: 50° C.
Eluents: A: Water+0.1% HCOOH
   B: ACN+0.1% HCOOH
Flow rate: 1.0 ml/min
Gradient: 2% to 98% B in 1.4 min

Method LCMS_IJ1:
Column: CORTECS™ C18 (2.1×50 mm, 2.7 μm)
Column temperature: 80° C.
Eluents: A: water+0.05% HCOOH+3.75 mM NH₄OAc
   B: ACN+0.04% HCOOH
Flow rate: 1.0 ml/min
Gradient: 5% to 98% B in 1.4 min

Method LCMS_JL1:
Column: XBRIDGE® BEH™ C18 (2.1×50 mm, 2.5 μm)
Column temperature: 80° C.
Eluents: A: water+5 mM NH₄OH
   B: ACN+5 mM NH₄OH
Flow rate: 1.0 ml/min
Gradient: 2% to 98% B in 9.4 min

Method LCMS_JL2:
Column: CORTECS™ C18 (2.1×50 mm, 2.7 μm)
Column temperature: 80° C.
Eluents: A: water+0.05% HCOOH+3.75 mM NH₄OAc
   B: iPrOH+0.05% HCOOH
Flow rate: 1.0 ml/min
Gradient: curved from 1% to 98% B in 1.7 min

Method LCMS_JL5:
Column: ASCENTIS® Express C18 (2.1×50 mm, 2.7 μm)
Column temperature: 80° C.
Eluents: A: water+4.76% iPrOH+0.05% HCOOH+3.75 mM NH₄OAc
   B: iPrOH+0.05% HCOOH
Flow rate: 0.6 ml/min
Gradient: 1% to 50% B in 1.4 min; 50% to 98% B in 0.3 min

Method LCMS_MLG1:
Column: ACQUITY UPLC® BEH C18 (2.1×50 mm, 1.7 μm)
Column temperature: 80° C.
Eluents: A: water+4.76% iPrOH+0.05% HCOOH+3.75 mM NH₄OAc
   B: iPrOH+0.05% HCOOH
Flow rate: 0.6 ml/min
Gradient: 1% to 98% B in 1.7 min

Method LCMS_MLG2:
Column: CORTECS™ C18 (2.1×50 mm, 2.7 μm)
Column temperature: 80° C.
Eluents: A: water+4.76% iPrOH+0.05% HCOOH+3.75 mM NH₄OAc
   B: iPrOH+0.05% HCOOH
Flow rate: 1.0 ml/min
Gradient: 1% to 50% B in 1.4 min; 50% to 98% in 0.3 min

Method LCMS_MLG3:
Column: XBRIDGE® BEH™ C18 (2.1×50 mm, 2.5 μm)
Column temperature: 80° C.
Eluents: A: water+5 mM NH₄OH
   B: ACN+5 mM NH₄OH
Flow rate: 1.0 ml/min
Gradient: 2% to 98% B in 1.4 min

Method LCMS_MLG4:
Column: ACQUITY UPLC® BE C18 (2.1×100 mm, 1.7 μm)
Column temperature: 80° C.
Eluents: A: water+4.76% iPrOH+0.05% HCOOH+3.75 mM NH₄OAc
   B: iPrOH+0.05% HCOOH
Flow rate: 0.4 ml/min
Gradient: 1% to 60% B in 8.4 min; 60% to 98% in 1.0 min

Method LCMS_MLG5:
Column: ACQUITY UPLC® BEH C18 (2.1×50 mm, 1.7 μm)
Column temperature: 80° C.
Eluents: A: water+0.05% HCOOH+3.75 mM NH₄OAc
   B: ACN+0.04% HCOOH
Flow rate: 1.0 ml/min
Gradient: 5% to 98% B in 1.4 min

Method LCMS_MLG7:
Column: CORTECS™ C18 (2.1×50 mm, 2.7 μm)
Column temperature: 80° C.
Eluents: A: water+4.76% iPrOH+0.05% HCOOH+3.75 mM NH₄OAc
   B: iPrOH+0.05% HCOOH
Flow rate: 1.0 ml/min
Gradient: 1% to 98% B in 0.5 min; 1.30 min 98% B

Method LCMS_MLG8:
Column: ASCENTIS® Express C18 (2.1×50 mm, 2.7 μm)
Column temperature: 80° C.
Eluents: A: water+4.76% iPrOH+0.05% HCOOH+3.75 mM NH₄OAc
   B: iPrOH+0.05% HCOOH
Flow rate: 1.0 ml/min
Gradient: 1% to 50% B in 1.4 min; 50% to 98% B in 0.3 min

Method LCMS_MLG9:
Column: ACQUITY UPLC® BEH C18 (2.1×50 mm, 1.7 μm)
Column temperature: 40° C.
Eluents: A: water+0.1% TFA
   B: ACN
Flow rate: 1.0 ml/min
Gradient: 5% to 98% B in 1.4 min

Method LCMS_MLG10:
Column: CORTECS™ C18 (2.1×50 mm, 2.7 μm)
Column temperature: 80° C.
Eluents: A: water+4.76% iPrOH+0.05% HCOOH+3.75 mM NH₄OAc
   B: iPrOH+0.05% HCOOH
Flow rate: 1.0 ml/min
Gradient: 0% to 50% B in 1.4 min; 50% to 98% B in 0.4 min; 0.1 min 98% B

Method LCMS_MLG-New-2:
Column: ACQUITY UPLC® BEH C18 (2.1×50 mm, 1.7 μm)
Column temperature: 80° C.
Eluents: A: water+4.76% iPrOH+0.05% HCOOH+3.75 mM NH₄OAc
   B: iPrOH+0.05% HCOOH
Flow rate: 1.0 ml/min
Gradient: 1% to 98% B in 1.7 min

Method LCMS_PL2:
Column: ACQUITY UPLC® HSS T3 (2.1×50 mm, 1.8 m)
Column temperature: 60° C.
Eluents: A: water+0.05% HCOOH+3.75 mM NH₄OAc
   B: ACN+0.04% HCOOH
Flow rate: 1.0 mL/min
Gradient: concave from 1% to 98% B in 1.4 min

Method LCMS WX2:
Column: Kinetex EVO C18 (2.1×30 mm, 5 m)
Column temperature: 50° C.
Eluents: A: water+0.0375% TFA
   B: ACN+0.01875% TFA
Flow rate: 1.5 ml/min
Gradient: 5% to 95% (B in 1.55 min)

Method LCMS WX3:
Column: Kinetex EVO C18 (2.1×30 mm, 5 m)
Column temperature: 50° C.
Eluents: A: water+0.0375% TFA
   B: ACN+0.01875% TFA
Flow rate: 1.5 ml/min
Gradient: 0% to 60% (B in 1.55 min)

Method LCMS WX4:
Column: Kinetex EVO C18 (2.1×30 mm, 5 μm)
Column temperature: 40° C.
Eluents: A: water+0.025% NH₃
   B: ACN
Flow rate: 1.5 ml/min
Gradient: 5% to 95% (B in 1.55 min)

Preparative Chromatography Methods:

Normal and reverse phase chromatography purifications were performed on a Biotage Isolera One system, or alternatively on a CombiFlash Rf200 or Rf+ system, or alternatively on an Interchim Puriflash 4250 system. Separations using SFC were performed using a Waters preparative SFC-100-MS system with either a Waters 2998 photodiode array detector or a Waters MS single quadrupole detection using MeOH as modifier. Generally, the back pressure was 120 bar, the flow 100 g C02/min and the column temperature 40° C. Reverse phase HPLC purifications were performed on a Waters HPLC preparative system with either a Waters 2998 photodiode 10 array detector or a Waters MS single quadrupole detection or alternatively, on a Gilson 281 (PHG012) system with dual UV wavelength detection system at 214 nm and 254 nm.

Achiral Preparative HPLC Methods:

Method with Basic Modifier:
Instrument: Gilson 281 (PHG012)
Column: Xtimate C18 (21.2×250 mm, 10 μm)
Column temperature: RT
Eluents: A: aq. NH₄HCO₃ (10 mM)
   B: ACN
Flow: 30 ml/min
Detection: MS and/or UV @ 254 nm, 214 nm

Method with acidic modifier:
Instrument: Gilson 281 (PHG012)
Column: Xtimate C18 (21.2×250 mm, 10 μm)
Column temperature: RT
Eluents: A: aq. TFA (0.1%)
   B: ACN
Flow: 30 ml/min
Detection: MS and/or UV @ 254 nm, 214 nm

Materials Used for Solid Phase Extraction:

The following solid phase extraction (SPE) cartridges were used according to manufacturers notice to generate the corresponding free base from different salts:

PL-HCO₃ MP SPE cartridges were purchased from Agilent StratosPhere—Ref: PL-HCO₃ MP-resin, 1.8 mmol/g, 100 A, 150-300 μm, 500 mg, 6 ml.

ISOLUTE® SCX SPE cartridges were purchased from BIOTAGE®—PART No. 530-0200-D, 2 g, 15 ml.

### Example 1. Synthesis of Targeting Ligase Binder, Linker, Linker Fragment and Targeting Ligand Intermediates

### Intermediate 1

### Tert-butyl 4-(piperidin-4-yloxy)piperidine-1-carboxylate

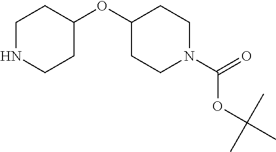 [SMILES: CC(C)(C)OC(=O)N1CCC(OC2CCNCC2)CC1]

### Step 1: tert-butyl 4-(pyridin-4-yloxy)piperidine-1-carboxylate

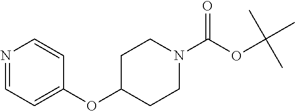 [SMILES: CC(C)(C)OC(=O)N1CCC(Oc2ccncc2)CC1]

To a 1 L round bottom flask were added pyridin-4-ol (20 g, 210 mmol), tert-butyl 4-hydroxypiperidine-1-carboxylate (57 g, 263 mmol), PPh₃ (72 g, 275 mmol) and THE (200 ml). The RM was stirred at 0° C. and DEAD (48 g, 275 mmol) was added dropwise over 60 min. The RM was allowed to reach RT and was then stirred at RT for 16 h. The mixture was concentrated, diluted with EtOAc (1 L) and stirred at 0° C. for 30 min. A solution of HCl (4 M) in 1,4-dioxane (100 ml) was added dropwise at 0° C. over 30 min and the mixture was stirred at 0° C. for 2 h. The mixture was filtered, the solids were washed with EtOAc (200 ml), the solids were redissolved in water (1 L), the aq. phase was extracted with EtOAc (3×200 mL) and the aq. phase was adjusted to pH=10 by the addition of an aq. solution of NaOH (1 M). The mixture was extracted with EtOAc (3×1 L) and the combined organic phases were dried over Na₂SO₄, yielding the title compound as a solid (32 g).

Method A: Rt=1.19 min; [M+H]⁺=279.

### Step 2: tert-butyl 4-(piperidin-4-yloxy)piperidine-1-carboxylate

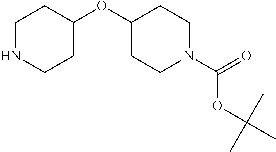 [SMILES: CC(C)(C)OC(=O)N1CCC(OC2CCNCC2)CC1]

To a 500 ml high pressure reactor were added tert-butyl 4-(pyridin-4-yloxy)piperidine-1-carboxylate (32 g, 115 mmol), Pd/C (10%, 9.6 g), EtOH (300 ml) and HOAc (30 ml). The mixture was stirred under H₂ atmosphere (60 bar) at 80° C. for 24 h. The mixture was cooled to RT and filtered. The filtrate was diluted with DCM (1 L) and the organic phase was washed with an aq. solution of NaOH (2 M, 2×200 mL), water (200 mL), brine (2×100 ml) and dried over Na₂SO₄, yielding the title compound as a solid (31 g).

Method B: Rt=1.96 min; [M+H]⁺=285.

### Intermediate 2

### Tert-butyl 4-(piperazin-1-ylmethyl)piperidine-1-carboxylate

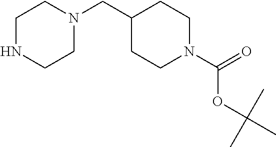 [SMILES: CC(C)(C)OC(=O)N1CCC(CN2CCNCC2)CC1]

### Step 1: tert-butyl 4-(((methylsulfonyl)oxy)methyl)piperidine-1-carboxylate

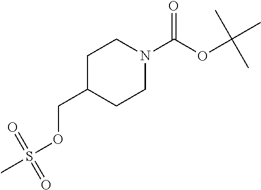 [SMILES: CC(C)(C)OC(=O)N1CCC(COS(C)(=O)=O)CC1]

To a 500 ml round bottom flask were added tert-butyl 4-(hydroxymethyl)piperidine-1-carboxylate (5 g, 23 mmol), DIEA (5.9 g, 46 mmol) and DCM (150 ml). The RM was stirred at RT for 10 min and MsCl (3 g, 27 mmol) was added dropwised over 5 min. The RM was stirred at RT for 1 h, added into DCM (300 ml), the organic phase was washed with water (3×100 ml) and dried over Na₂SO₄, yielding the title compound as a solid (7 g).

Method C: Rt=1.74 min; [M+Na]⁺=316.

### Step 2: tert-butyl 4-(piperazin-1-ylmethyl)piperidine-1-carboxylate

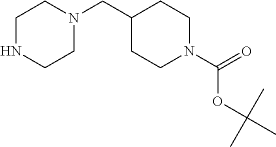 [SMILES: CC(C)(C)OC(=O)N1CCC(CN2CCNCC2)CC1]

To a 250 ml round bottom flask were added tert-butyl 4-(((methylsulfonyl)oxy)methyl)piperidine-1-carboxylate (2 g, 6.8 mmol), piperazine (860 mg, 10 mmol), KI (100 mg, 0.68 mmol), K₂CO₃ (2.8 g, 20 mmol) and ACN (120 ml). The RM was stirred at 80° C. for 16 h, filtered, the filtrate was concentrated and the residue was purified by reversed phase chromatography on a Biotage Agela C18 column (120 g, spherical 20-35 μm, 100 Å) eluting with ACN (from 5% to 40%) in an aq. solution of NH₄HCO₃ (0.1%) yielding the title compound as an oil (410 mg).

Method B: Rt=1.74 min; [M+H]⁺=284.

### Intermediate 3

### 5-Bromo-1,3,4-trimethylpyridin-2(1H)-one

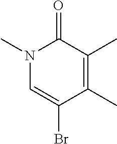

### Step 1: 5-bromo-3,4-dimethylpyridin-2(1H)-one

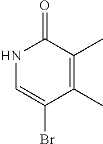

To a 250 ml round bottom flask were added 5-bromo-3,4-dimethylpyridin-2-amine (4.42 g, 21.32 mmol), H₂SO₄ (11.5 ml, 211 mmol) and water (40 ml). A solution of NaNO₂ (2.06 g, 29.9 mmol) in water (3 ml) was added dropwise at 0° C. and the RM was stirred at RT overnight. NaNO₂ (2.06 g, 29.9 mmol) was added portionwise and the RM was stirred at RT for 1 h. The RM was filtered, the solids were washed with water and dried, yielding the title compound as a solid (2.53 g).

Method D: Rt=0.66 min; [M+H]⁺=202, 204.

### Step 2: 5-bromo-1,3,4-trimethylpyridin-2(1H)-one

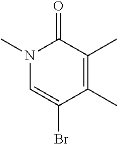

To a 50 ml round bottom flask were added 5-bromo-3,4-dimethylpyridin-2(1H)-one (2.53 mg, 12.25 mmol), K₂CO₃ (4274 mg, 30.6 mmol) and THE (25 ml). Methyl iodide (0.860 ml, 13.47 mmol) was added dropwise and the RM was stirred at RT overnight. The RM was filtered, the filtrate was concentrated and the residue purified by chromatography on silica gel eluting with EtOAc (from 0% to 100%) in CHX, yielding the title compound as a solid (2.47 g).

Method D: Rt=0.75 min; [M+H]⁺=216, 218.

### Intermediate 4

### 1,3-Dimethyl-5-(4,4,5,5-tetramethyl-1,3,2-dioxaborolan-2-yl)pyridin-2(1H)-one

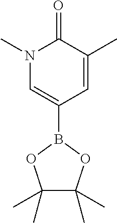

To a 100 ml round bottom flask were added under an argon atmosphere 5-bromo-1,3-dimethylpyridin-2(1H)-one (2.1 g, 10.39 mmol), BISPIN (3.7 g, 14.55 mmol), KOAc (2.04 g, 20.79 mmol) and 1,4-dioxane (50 ml). Solid PdCl₂(dppf)-CH₂Cl₂ (380 mg, 0.520 mmol) was added and the RM was stirred at 90° C. for 5 h. The RM was cooled to RT, filtered over CELITE®, concentrated and the residue was taken up in a mixture of DCM and water. The organic phase was dried over MgSO₄ and the residue was purified by chromatography on silica gel eluting with EtOAc (from 0% to 63%) in CHX, yielding the title compound as a solid (2.69 g).

Method D: Rt=0.93 min; [M+H]⁺=250.

### Intermediate 5

### 4-Bromo-2-methyl-2,7-naphthyridin-1(2H)-one

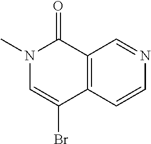

To a 1 L round bottom flask were added 4-bromo-2,7-naphthyridin-1(2H)-one (9 g, 40 mmol) and DMF (200 ml) and the mixture was stirred at 0° C. for 1 h. Solid NaH (60% dispersion in mineral oil, 3.2 g, 80 mmol) was added and the RM was stirred at 0° C. for 30 min. Iodomethane (17 g, 120 mmol) was added dropwise over 5 min, the RM was allowed to warm to RT and stirring was continued at RT for 16 h. The mixture was added into water (500 ml) and the aq. layer was extracted with EtOAc (4×300 mL), the combined organic phases were washed with brine (200 mL) and dried over Na₂SO₄, yielding the title compound as a solid (7 g).

Method A: Rt=1.43 min; [M+H]⁺=239, 241.

### Intermediate 6

### 4-Bromo-2,6-dimethoxyphenol

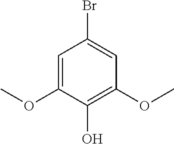

To a 100 ml round bottom flask were added 2,6-dimethoxyphenol (2 g, 12.97 mmol) and CHCl₃ (20 ml) under argon. EtOH (0.16 ml, 2.74 mmol) and NaH (60% in mineral oil, 10.4 mg, 0.26 mmol) were added and the mixture was cooled to −78° C. Solid N-bromosuccinimide (2.3 g, 12.92 mmol) was added portionswise and the RM was stirred at −78° C. for 1 h. The cooling bath was removed, the RM was stirred at RT for 30 minutes and then at 65° C. for 5 min. The RM was cooled to RT and concentrated. The residue was taken up in Et₂O and filtered. The filtrate was concentrated and recristallized in heptane at 85° C., yielding the title compound as a solid (1.14 g).

¹H NMR (400 MHz, DMSO-d6) δ 8.57 (s, 1H), 6.78 (s, 2H), 3.76 (s, 6H).

### Intermediate 7

### 2,6-Dimethoxy-4-(4,4,5,5-tetramethyl-1,3,2-dioxaborolan-2-yl)benzaldehyde

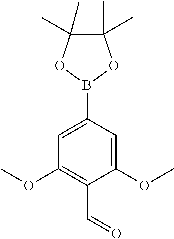

To a 500 ml round bottom flask were added under an argon atmosphere 4-bromo-2,6-dimethoxybenzaldehyde (8 g, 31.7 mmol), BISPIN (10 g, 39.4 mmol), dppf (527 mg, 0.950 mmol), KOAc (9.32 g, 95 mmol) and 1,4-dioxane (100 ml). Solid PdCl₂(dppf) (695 mg, 0.950 mmol) was added and the RM was stirred at 90° C. overnight. The RM was cooled to RT, filtered over CELITE® and the solids were washed with EtOAc. The combined filtrates were washed with an aq. solution of HCl (0.1 N) and brine, dried over MgSO₄ and the residue was purified by chromatography on silica gel eluting with EtOAc (from 5% to 100%) in CHX, yielding the title compound as a solid (7.42 g).

Method D: Rt=1.12 min; [M+H]⁺=293.

### Intermediate 8

### 4-(1,5-Dimethyl-6-oxo-1,6-dihydropyridin-3-yl)-2,5-dimethoxybenzaldehyde

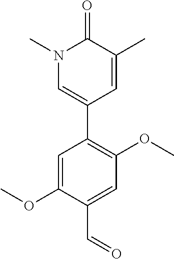

To a 100 ml round bottom flask were added under an argon atmosphere 4-bromo-2,5-dimethoxybenzaldehyde (1.054 g, 4.21 mmol), 1,3-dimethyl-5-(4,4,5,5-tetramethyl-1,3,2-dioxaborolan-2-yl) pyridin-2(1H)-one (intermediate 4, 1.0 g, 3.97 mmol), an aq. solution of NaOAc (2 M, 4.97 ml, 9.94 mmol) and DMF (25 ml). Solid PdCl2(dppf)-CH2Cl2(294 mg, 0.367 mmol) was added and the RM was stirred at 100° C. for 1 h. The mixture was cooled to RT, filtered through CELITE®, the filtrate was concentrated and the residue was triturated in a mixture of DCM, MTBE and water and filtered. The solids were washed with DCM, EtOAc and dried, yielding the title compound a solid (1.109 g).

Method D: Rt=0.84 min; [M+H]=288.

### Intermediate 9:

### 4-(1,5-Dimethyl-6-oxo-1,6-dihydropyridin-3-yl)-2.6-dimethoxybenzaldehyde

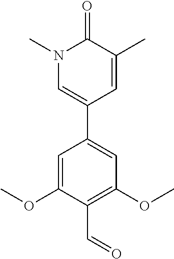

To a 100 ml round bottom flask were added under an argon atmosphere 4-bromo-2,6-dimethoxybenzaldehyde (1.31 g, 5.18 mmol), 1,3-dimethyl-5-(4,4,5,5-tetramethyl-1,3,2-dioxaborolan-2-yl)pyridin-2(1H)-one (intermediate 4, 1.5 g, 4.94 mmol), an aq. solution of NaOAc (2 M, 6.17 ml, 12.34 mmol) and DMF (15 ml). Solid PdCl₂(dppf)-CH₂Cl₂(365 mg, 0.494 mmol) was added and the RM was stirred at 100° C. for 1 h. The mixture was cooled to RT, filtered through CELITE®, the filtrate was concentrated, the residue was taken up in a mixture of EtOAc and water and filtered, yielding the title compound as a solid (599 mg).

Method D: Rt=0.73 min; [M+H]⁺=288.

### Intermediate 10

### 4-(1,5-Dimethyl-6-oxo-1,6-dihydropyridin-3-yl)-2-methoxybenzoic Acid

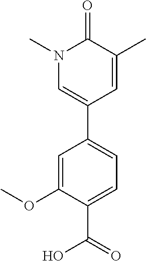

To a 50 ml round bottom flask were added under an argon atmosphere 4-bromo-2-methoxybenzoic acid (500 mg, 2.164 mmol), 1,3-dimethyl-5-(4,4,5,5-tetramethyl-1,3,2-dioxaborolan-2-yl)pyridin-2(1H)-one (intermediate 4, 516 mg, 2.051 mmol), an aq. solution of NaOAc (2 M, 2.56 ml, 5.13 mmol) and DMF (10 ml). Solid PdCl₂(dppf)-CH₂Cl₂(152 mg, 0.205 mmol) was added and the RM was stirred at 100° C. for 1 h. The mixture was cooled to RT, filtered through CELITE®, the filtrate was concentrated and the residue was taken up in a mixture of DCM and water. The organic phase was separated and filtered, the filtrate evaporated and the residue was purified by reversed phase chromatography on a REDISEP® Gold HP C18 column (15.5 g) eluting with ACN (from 2% to 100%) in an aq. solution of TFA (0.1%) yielding the title compound as a solid (293 mg).

Method D: Rt=0.65 min; [M+H]⁺=274.

### Intermediate 11

### 2,6-Dimethoxy-4-(2-methyl-1-oxo-1,2-dihydro-2,7-naphthyridin-4-yl)benzaldehyde

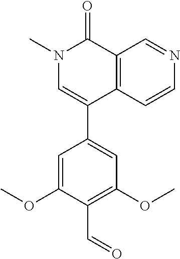 [SMILES: COc1cc(-c2cn(C)c(=O)c3cnccc23)cc(OC)c1C=O]

To a 250 ml round bottom flask were added under an argon atmosphere 4-bromo-2-methyl-2,7-naphthyridin-1(2H)-one (intermediate 5, 950 mg, 3.97 mmol), 2,6-dimethoxy-4-(4,4,5,5-tetramethyl-1,3,2-dioxaborolan-2-yl)benzaldehyde (intermediate 7, 1.4 g, 4.77 mmol), Na₂CO₃ (1.3 g, 11.91 mmol), 1,4-dioxane (10 ml), water (2.5 ml) and PdCl₂(dppf) (145 mg, 0.2 mmol). The RM was stirred at 100° C. for 16 h. The mixture was added into EtOAc (200 ml), the organic phase was washed with brine, concentrated and the residue was purified by chromatography on silica gel eluting with EA (from 0% to 50%) in PE, yielding the title compound as a solid (960 mg).

Method A: Rt=1.26 min; [M+H]⁺=325.

### Intermediate 12

### 2,5-Dimethoxy-4-(2-methyl-1-oxo-1,2-dihydro-2,7-naphthyridin-4-yl)benzaldehyde

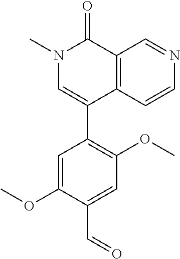 [SMILES: COc1cc(-c2cn(C)c(=O)c3cnccc23)c(OC)cc1C=O]

### Step 1: 2,5-dimethoxy-4-(4,4,5,5-tetramethyl-1,3,2-dioxaborolan-2-yl)benzaldehyde

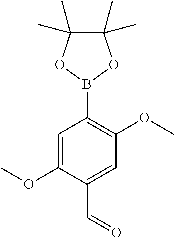

To a 250 ml round bottom flask were added under an argon atmosphere 4-bromo-2,5-dimethoxybenzaldehyde (5 g, 20.4 mmol), BISPIN (6.2 g, 24.5 mmol), KOAc (6.0 g, 61.2 mmol), 1,4-dioxane (50 ml) and PdCl₂(dppf) (732 mg, 1 mmol) and the RM was stirred at 90° C. for 16 h. The mixture was concentrated and the residue was purified by chromatography on silica gel eluting with MeOH (from 0% to 15%) in DCM, yielding the title compound as a yellow solid (4.7 g).

Method A: Rt=1.76 min; [M+H]⁺ 293.

### Step 2: 2,5-dimethoxy-4-(2-methyl-1-oxo-1,2-dihydro-2,7-naphthyridin-4-yl)benzaldehyde

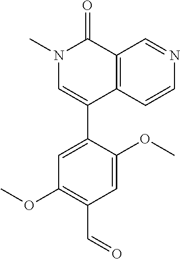

To a 500 ml round bottom flask were added under an argon atmosphere 4-bromo-2-methyl-2,7-naphthyridin-1(2H)-one (intermediate 5, 7.5 g, 31.4 mmol), 2,5-dimethoxy-4-(4,4,5,5-tetramethyl-1,3,2-dioxaborolan-2-yl)benzaldehyde (13.7 g, 47.1 mmol), Na₂CO₃ (10 g, 94.2 mmol), 1,4-dioxane (160 ml), water (40 ml) and PdCl₂(dppf) (1.15 g, 1.57 mmol). The RM was stirred at 100° C. for 2 h. The mixture was added into water (100 mL), extracted with DCM (3×200 mL), the combined organic phases were washed with brine (100 mL), dried over Na₂SO₄ and concentrated. The residue was titrated in cold MTBE, the mixture was filtered and the solids were dried, yielding the title compound as a solid (7.2 g).

Method F: Rt=1.62 min; [M+H]⁺=325.

### Intermediate 13

### 3-(2,6-Dimethoxy-4-(2-methyl-1-oxo-1,2-dihydro-2,7-naphthyridin-4-yl)phenyl)propanal

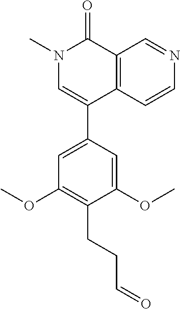

### Step 1: methyl (E)-3-(4-bromo-2,6-dimethoxyphenyl)acrylate

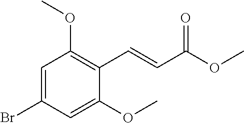 [SMILES: COC(=O)/C=C/c1c(OC)cc(Br)cc1OC]

To a 250 ml round bottom flask were added 4-bromo-2,6-dimethoxybenzaldehyde (3 g, 12.24 mmol), THE (100 ml) and NaH (60% dispersion in mineral oil, 2 g, 48.97 mmol). The RM was stirred at RT for 30 min and cooled to 0° C. A solution of methyl 2-(dimethoxyphosphoryl)acetate in THF (20 ml) was added and the RM was allowed to reach RT and stirring was continued for 16 h. The mixture was again cooled to 0° C. and an aq. sat. solution of NH₄Cl was added. The mixture was extracted with EtOAc (2×50 ml), the combined organic phases were concentrated and the residue was purified by chromatography on silica gel eluting with EtOAc (from 0% to 10%) in PE, yielding the title compound as a solid (3.3 g).

Method G: Rt=2.06 min; [M+H]⁺=301.

### Step 2: methyl (E)-3-(2,6-dimethoxy-4-(4,4,5,5-tetramethyl-1,3,2-dioxaborolan-2-yl)phenyl)acrylate

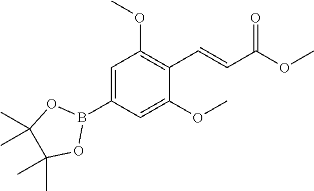

To a 100 ml round bottom flask were added under an argon atmosphere methyl (E)-3-(4-bromo-2,6-dimethoxyphenyl)acrylate (1.5 g, 4.98 mmol), BISPIN (1.52 g, 5.98 mmol), KOAc (1.47 g, 14.94 mmol), PdCl₂(dppf) (37 mg, 0.05 mmol) and 1,4-dioxane (40 ml). The RM was stirred at 90° C. for 16 h. The mixture was filtered, the solids were washed with EtOAc (50 ml) and the combined filtrates were concentrated and the residue was purified by chromatography on silica gel eluting with EtOAc (from 0% to 15%) in PE, yielding the title compound as a solid (1.38 g).

Method G: Rt=2.15 min; [M+H]⁺=349.

### Step 3: methyl 3-(2,6-dimethoxy-4-(4,4,5,5-tetramethyl-1,3,2-dioxaborolan-2-yl)phenyl)propanoate

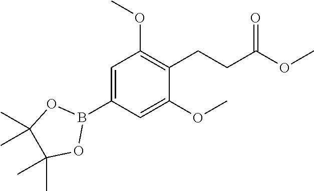

To a 50 ml round bottom flask were added methyl (E)-3-(2,6-dimethoxy-4-(4,4,5,5-tetramethyl-1,3,2-dioxaborolan-2-yl)phenyl)acrylate (1.3 g, 3.74 mmol), Pd/C (10%, 200 mg) and MeOH (30 ml). The RM was stirred under a H₂ atmosphere (1 bar) at 50° C. for 2 h, filtered and the filtrate was concentrated, yielding the title compound as a solid (1.2 g).

Method G: Rt=2.12 min; [M+H]⁺=351.

### Step 4: (4-(3-hydroxypropyl)-3,5-dimethoxyphenyl)boronic Acid

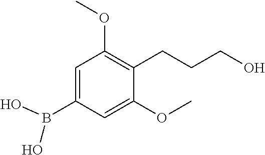 [SMILES: COc1cc(B(O)O)cc(OC)c1CCCO]

To a 50 ml round bottom flask were added methyl 3-(2,6-dimethoxy-4-(4,4,5,5-tetramethyl-1,3,2-dioxaborolan-2-yl)phenyl)propanoate (1.2 g, 3.43 mmol) and THF (30 ml). The RM was cooled to 0° C. and LiAlH₄ (390 mg, 10.28 mmol) was added portionswise. The RM was stirred at RT for 6 h, cooled to 0° C. and water was carefully added. The RM was extracted with EtOAc (2×50 ml) and the combined organic phases were concentrated, yielding the title compound as a solid (750 mg).

Method G: Rt=1.97 min; [M+H]⁺=241.

### Step 5: 4-(4-(3-hydroxypropyl)-3,5-dimethoxyphenyl)-2-methyl-2,7-naphthyridin-1(2H)-one

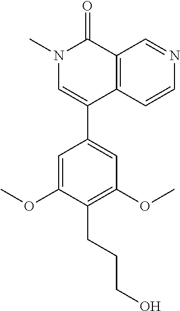

To a 50 ml round bottom flask were added under an argon atmosphere (4-(3-hydroxypropyl)-3,5-dimethoxyphenyl)boronic acid (650 mg, 2.71 mmol), 4-bromo-2-methyl-2,7-naphthyridin-1(2H)-one (intermediate 5, 647 mg, 2.71 mmol), Na₂CO₃ (720 mg, 6.77 mmol), PdCl₂(dppf) (99 mg, 0.14 mmol), 1,4-dioxane (15 ml) and water (3 ml). The RM was stirred at 80° C. for 16 h, filtered, the solids were washed with EtOAc (200 ml), the combined organic phases were dried over MgSO₄ and concentrated, yielding the title compound as a solid (650 mg), which was directly used for the next step without further purification.

Method G: Rt=1.64 min; [M+H]⁺=355.

### Step 6: 3-(2,6-dimethoxy-4-(2-methyl-1-oxo-1,2-dihydro-2,7-naphthyridin-4-yl)phenyl)propanal

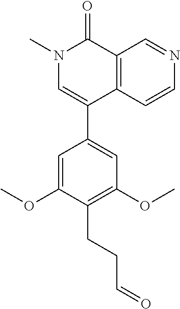

To a 25 ml round bottom flask were added 4-(4-(3-hydroxypropyl)-3,5-dimethoxyphenyl)-2-methyl-2,7-naphthyridin-1(2H)-one (300 mg, 0.85 mmol), IBX (476 mg, 1.7 mmol) and DMSO (5 ml). The RM was stirred at 50° C. for 4 h. An aq. sat. solution of NaCl (80 ml) was added and the aq. phase was extracted with EtOAc (3×50 ml), the combined organic phases were concentrated, yielding the title compound as a solid (270 mg), which was directly used for the next step without further purification.

Method G: Rt=1.73 min; [M+H]⁺=353.

### Intermediate 14

### 2-(2,6-Dimethoxy-4-(2-methyl-1-oxo-1,2-dihydro-2,7-naphthyridin-4-yl)phenyl)acetaldehyde

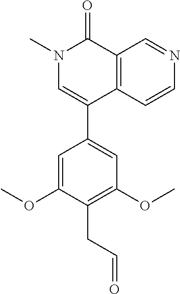

### Step 1: 4-(3,5-dimethoxy-4-(2-methoxyvinyl)phenyl)-2-methyl-2,7-naphthyridin-1(2H)-one

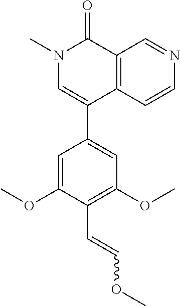

To a 250 ml round bottom flask were added (methoxymethyl)triphenylphosphonium chloride (3.08 g, 9 mmol), t-BuOK (1.34 g, 12 mmol) and TH (70 ml). The RM was stirred at 0° C. for 30 min, solid 2,6-dimethoxy-4-(2-methyl-1-oxo-1,2-dihydro-2,7-naphthyridin-4-yl)benzaldehyde (intermediate 11, 972 mg, 3 mmol) was added and the RM was stirred at 0° C. for 5 h, then concentrated and the residue was purified by chromatography on silica gel eluting with EtOAc (from 0% to 100%) in PE, yielding the title compounds as a solid (1 g).

Method E: Rt=1.47 min; [M+H]⁺=353.

### Step 2: 2-(2,6-dimethoxy-4-(2-methyl-1-oxo-1,2-dihydro-2,7-naphthyridin-4-yl)phenyl)acetaldehyde

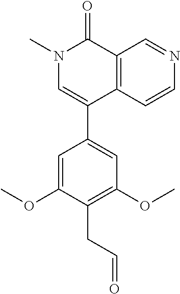

To a 50 ml round bottom flask were added 4-(3,5-dimethoxy-4-(2-methoxyvinyl)phenyl)-2-methyl-2,7-naphthyridin-1(2H)-one (500 mg, 1.42 mmol), an aq. solution of HCl (2 M, 3 ml, 6 mmol) and acetone (15 ml). The RM was stirred at 65° C. for 5 h, concentrated and the residue was purified by chromatography on a Biotage Agela C18 column (120 g, spherical 20-35 μm, 100 Å) eluting with ACN (from 5% to 95%) in an aq. solution of NH₄HCO₃ (0.1%), yielding the title compound as a solid (420 mg).

Method E: Rt=1.44 min; [M+H]⁺=339.

### Intermediate 15

### 2-(2,5-Dimethoxy-4-(2-methyl-1-oxo-1,2-dihydro-2,7-naphthyridin-4-yl)phenyl)acetaldehyde

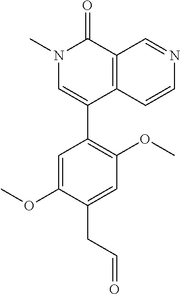

### Step 1: 4-(2,5-dimethoxy-4-(2-methoxyvinyl)phenyl)-2-methyl-2,7-naphthyridin-1(2H)-one

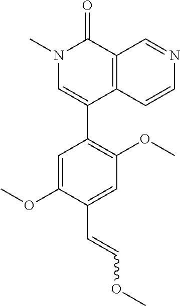

To a 100 ml round bottom flask were added (methoxymethyl)triphenylphosphonium chloride (648 mg, 2 mmol) and THE (15 ml), the mixture was cooled to 0° C. and solid t-BuOK (900 mg, 8 mmol) was added. The RM was stirred at 0° C. for 30 min, solid 2,5-dimethoxy-4-(2-methyl-1-oxo-1,2-dihydro-2,7-naphthyridin-4-yl)benzaldehyde (intermediate 12, 648 mg, 2 mmol) was added portionwise and the RM was stirred at 70° C. for 16 h. The solvent was removed, the residue was added into water (50 mL), the mixture was extracted with EtOAc (3×50 mL), the combined organic phases were dried over Na₂SO₄ and the residue was purified by chromatography on silica gel eluting with EtOAc (from 0% to 35%) in PE, yielding the title compound as a solid (1.3 g).

Method G. Rt=1.82 min; [M+H]⁺=353.

### Step 2: 2-(2,5-dimethoxy-4-(2-methyl-1-oxo-1,2-dihydro-2,7-naphthyridin-4-yl)phenyl)acetaldehyde

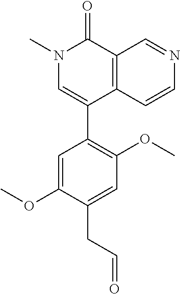

To a 100 ml round bottom flask was added 4-(2,5-dimethoxy-4-(2-methoxyvinyl)phenyl)-2-methyl-2,7-naphthyridin-1(2H)-one (1.05 g, 3 mmol) and acetone (30 ml). The mixture was stirred at RT for 5 min, an aq. solution of HCl (2 M, 4 ml) was added and the RM was stirred at 60° C. for 4 h. The solvents were removed and the residue was purified by preparative HPLC on an XBridge C18 column (250×21.2 mm, 10 μm) eluting with ACN (from 5% to 95%) in an aq. solution of NH₄HCO₃ (10 mM), yielding the title compound as a solid (520 mg).

Method B: Rt=1.36 min; [M+H]⁺=339.

### Intermediate 16

### Tert-butyl 4-(2-methoxy-4-(4,4,5,5-tetramethyl-1,3,2-dioxaborolan-2-yl)phenoxy)piperidine-1-carboxylate

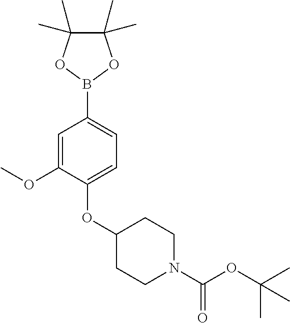 [SMILES: COc1cc(B2OC(C)(C)C(C)(C)O2)ccc1OC1CCN(C(=O)OC(C)(C)C)CC1]

To a 500 ml round bottom flask were added under an argon atmosphere 2-methoxy-4-(4,4,5,5-tetramethyl-1,3,2-dioxaborolan-2-yl)phenol (6 g, 23.99 mmol), tert-butyl 4-hydroxypiperidine-1-carboxylate (5.5 g, 26.80 mmol), PPh₃ (7 g, 26.70 mmol) and THF (120 ml). DIAD (5.6 ml, 28.80 mmol) was slowly added and the RM was stirred at RT overnight. The mixture was diluted with EtOAc and an aq. sat. solution of NaHCO₃ was added. The organic phase was washed with brine, dried over MgSO₄ and the residue was purified by chromatography on silica gel eluting with EtOAc (from 0% to 20%) in CHX, yielding the title compound as a solid (9.1 g).

Method D: Rt=1.41 min; [M+H]⁺=434.

### Intermediate 17

### Tert-butyl 4-(2,6-dimethoxy-4-(4,4,5,5-tetramethyl-1,3,2-dioxaborolan-2-yl)phenoxy)piperidine-1-carboxylate

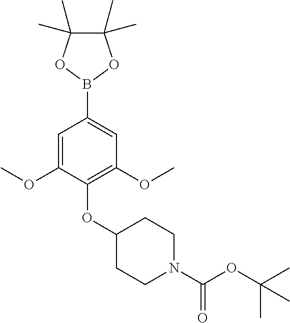 [SMILES: COc1cc(B2OC(C)(C)C(C)(C)O2)cc(OC)c1OC1CCN(C(=O)OC(C)(C)C)CC1]

### Step 1: tert-butyl 4-(4-bromo-2,6-dimethoxyphenoxy)piperidine-1-carboxylate

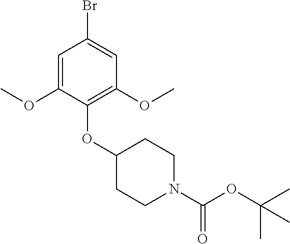 [SMILES: COc1cc(Br)cc(OC)c1OC1CCN(C(=O)OC(C)(C)C)CC1]

To a 100 ml round bottom flask were added under an argon atmosphere 4-bromo-2,6-dimethoxyphenol (intermediate 6, 406 mg, 1.481 mmol), tert-butyl 4-hydroxypiperidine-1-carboxylate (335 mg, 1.629 mmol), PPh₃ (466 mg, 1.777 mmol) and THE (8 ml). DIAD (0.345 ml, 1.777 mmol) was slowly added and the RM was stirred at RT overnight. The RM was diluted with EtOAc and added into an aq. sat. solution of NaHCO₃. The organic phase was washed with brine, dried over MgSO₄ and the residue was purified by chromatography on silica gel eluting with EtOAc (from 0% to 20%) in CHX, yielding the title compound as a solid (506 mg).

Method D: Rt=1.35 min; [M-tBu+H]⁺=360, 362.

### Step 2: tert-butyl 4-(2,6-dimethoxy-4-(4,4,5,5-tetramethyl-1,3,2-dioxaborolan-2-yl)phenoxy)piperidine-1-carboxylate

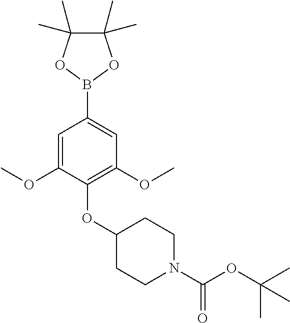 [SMILES: COc1cc(B2OC(C)(C)C(C)(C)O2)cc(OC)c1OC1CCN(C(=O)OC(C)(C)C)CC1]

To a 50 ml round bottom flask were added under an argon atmosphere tert-butyl 4-(4-bromo-2,6-dimethoxyphenoxy)piperidine-1-carboxylate (500 mg, 1.165 mmol), BISPIN (414 mg, 1.631 mmol), KOAc (229 mg, 2.330 mmol) and 1,4-dioxane (10 ml). Solid PdCl₂(dppf)-CH₂C2 (43 mg, 0.059 mmol) was added and the RM was stirred at 90° C. for 6 h. The mixture was filtered, the solvents of the filtrate were removed and the residue was purified by chromatography on silica gel eluting with EtOAc (from 0% to 25%) in CHX, yielding the title compound as a solid (407 mg).

Method D: Rt=1.43 min; [M+H]⁺=464.

### Intermediate 18

### Tert-butyl 4-((2,6-dimethoxy-4-(4,4,5,5-tetramethyl-1,3,2-dioxaborolan-2-yl)phenoxy)methyl)piperidine-1-carboxylate

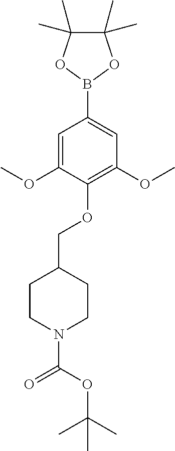

### Step 1: tert-butyl 4-((4-bromo-2,6-dimethoxyphenoxy)methyl)piperidine-1-carboxylate

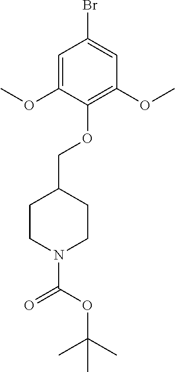

To a 100 ml round bottom flask were added under an argon atmosphere 4-bromo-2,6-dimethoxyphenol (intermediate 6, 293 mg, 1.157 mmol), tert-butyl 4-(hydroxymethyl)piperidine-1-carboxylate (280 mg, 1.272 mmol), PPh₃ (364 mg, 1.388 mmol) and THE (7 ml). DIAD (0.270 ml, 1.388 mmol) was slowly added and the RM was stirred at RT overnight. The RM was diluted with EtOAc and an aq. sat. solution of NaHCO₃ was added. The organic phase was washed with brine, dried over MgSO₄ and the residue was purified by chromatography on silica gel eluting with EtOAc (from 0% to 15%) in CHX, yielding the title compound as a solid (431 mg).

Method D: Rt=1.42 min; [M-tBu+H]⁺=374, 376.

### Step 2. tert-butyl 4-((2,6-dimethoxy-4-(4,4,5,5-tetramethyl-1,3,2-dioxaborolan-2-yl)phenoxy)methyl)piperidine-1-carboxylate

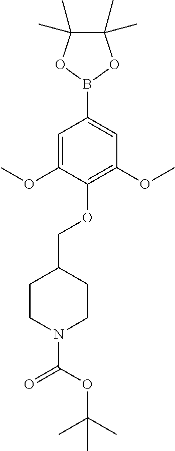

To a 50 ml round bottom flask were added under argon atmosphere tert-butyl 4-((4-bromo-2,6-dimethoxyphenoxy)methyl)piperidine-1-carboxylate (420 mg, 0.947 mmol), BISPIN (361 mg, 1.420 mmol), KOAc (279 mg, 2.84 mmol) and DME (8 ml). Solid PdCl₂(dppf)-CH₂Cl₂ (35 mg, 0.047 mmol) was added and the RM was stirred at 100° C. for 20 h. The RM was cooled to RT, filtered over CELITE® and the solids were washed with EtOAc. The combined filtrates were concentrated and the residue was purified by chromatography on silica gel eluting with EtOAc (from 0% to 35%) in CHX, yielding the title compound as a solid (382 mg).

Method D: Rt=1.49 min; [M+H]⁺=478.

### Intermediate 19

### 5-(3-Methoxy-4-((4-(piperidin-4-ylmethyl)piperazin-1-yl)methyl)phenyl)-1,3,4-trimethylpyridin-2(1H)-one

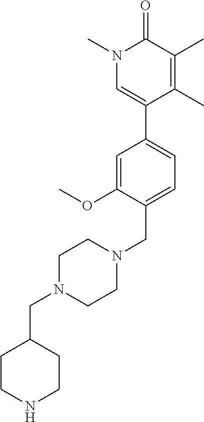 [SMILES: COc1cc(-c2cn(C)c(=O)c(C)c2C)ccc1CN1CCN(CC2CCNCC2)CC1]

### Step 1: 2-methoxy-4-(1,4,5-trimethyl-6-oxo-1,6-dihydropyridin-3-yl)benzaldehyde

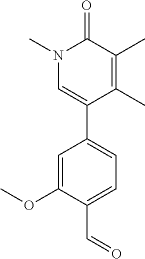

To a 25 ml round bottom flask were added under an argon atmosphere (4-formyl-3-methoxyphenyl)boronic acid (300 mg, 1.634 mmol), 5-bromo-1,3,4-trimethylpyridin-2(1H)-one (intermediate 3, 300 mg, 1.388 mmol), an aq. solution of NaOAc (2 M, 2.083 ml, 4.17 mmol) and DMF (7.5 ml). Solid PdCl₂(dppf)-CH₂Cl₂ (103 mg, 0.139 mmol) was added and the RM was stirred at 100° C. for 1 h. The mixture was cooled to RT, filtered through CELITE®, the filtrate was concentrated and the residue purified by reversed phase chromatography on a REDISEP® Gold HP C18 column (15.5 g) eluting with ACN (from 2% to 100%) in an aq. solution of TFA (0.1%), yielding the title compound as a solid (300 mg).

Method D: Rt=0.84 min; [M+H]⁺=272.

### Step 2: tert-butyl 4-(2-methoxy-4-(1,4,5-trimethyl-6-oxo-1,6-dihydropyridin-3-yl)benzyl)piperazine-1-carboxylate

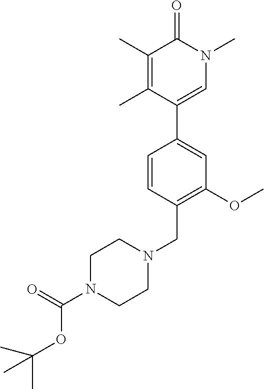 [SMILES: COc1cc(-c2cn(C)c(=O)c(C)c2C)ccc1CN1CCN(C(=O)OC(C)(C)C)CC1]

To a 10 ml round bottom flask were added tert-butyl piperazine-1-carboxylate (283 mg, 1.517 mmol), HOAc (0.059 ml, 1.029 mmol), NaOAc (124 mg, 1.517 mmol) and DCM (10 ml). The RM was stirred at 0° C. for 10 min, 2-methoxy-4-(1,4,5-trimethyl-6-oxo-1,6-dihydropyridin-3-yl)benzaldehyde (294 mg, 1.084 mmol) was added and the RM was stirred at RT for 30 min. Solid NaBH(OAc)₃ (111 mg, 0.552 mmol) was added and stirring of the RM was continued overnight. Solid NaBH(OAc)₃ (459 mg, 2.167 mmol) was added and stirring was continued at RT overnight. The mixture was concentrated, yielding a solid residue (498 mg) containing the title compound, which was directly used for next step without further purification.

Method D: Rt=0.72 min; [M+H]⁺=442.

### Step 3: 5-(3-methoxy-4-(piperazin-1-ylmethyl)phenyl)-1,3,4-trimethylpyridin-2(1H)-one

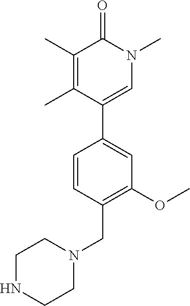 [SMILES: COc1cc(-c2cn(C)c(=O)c(C)c2C)ccc1CN1CCNCC1]

To a 25 ml round bottom flask were added tert-butyl 4-(2-methoxy-4-(1,4,5-trimethyl-6-oxo-1,6-dihydropyridin-3-yl)benzyl)piperazine-1-carboxylate (1.084 mmol), TFA (1 ml, 12.98 mmol) and DCM (4 ml). The RM was stirred at RT for 2 h, then concentrated and the residue was purified by reversed phase chromatography on a REDISEP® Gold HP C18 column (15.5 g) eluting with ACN (from 2% to 100%) in an aq. solution of TFA (0.1%), yielding the corresponding TFA salt of the title compound as a solid (620 mg).

Method D: Rt=0.49 min; [M+H]⁺=342.

### Step 4: tert-butyl 4-((4-(2-methoxy-4-(1,4,5-trimethyl-6-oxo-1,6-dihydropyridin-3-yl)benzyl)piperazin-1-yl)methyl)piperidine-1-carboxylate

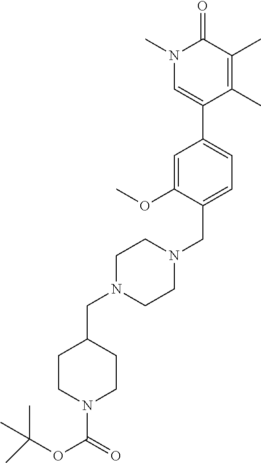 [SMILES: COc1cc(-c2cn(C)c(=O)c(C)c2C)ccc1CN1CCN(CC2CCN(C(=O)OC(C)(C)C)CC2)CC1]

To a 25 ml round bottom flask were added 5-(3-methoxy-4-(piperazin-1-ylmethyl)phenyl)-1,3,4-trimethylpyridin-2(1H)-one TFA salt (620 mg, 1.089 mmol), tert-butyl 4-formylpiperidine-1-carboxylate (279 mg, 1.306 mmol), TEA (0.500 ml, 3.59 mmol), a solution of ZnCl₂ (0.5 M) in THE (2.5 ml, 1.250 mmol) and MeOH (6 ml). The RM was stirred at RT for 7 h, solid NaBH₃CN (75 mg, 1.198 mmol) was added and the RM was stirred at RT overnight. The mixture was concentrated, yielding a solid residue (587 mg) containing the title compound, which was directly used for next step without further purification.

Method D: Rt=0.80 min; [M+H]⁺=539.

### Step 5: 5-(3-methoxy-4-((4-(piperidin-4-ylmethyl)piperazin-1-yl)methyl)phenyl)-1,3,4-trimethylpyridin-2(1H)-one

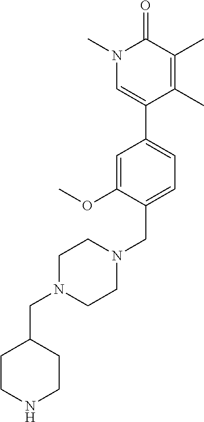 [SMILES: COc1cc(-c2cn(C)c(=O)c(C)c2C)ccc1CN1CCN(CC2CCNCC2)CC1]

To a 10 ml round bottom flask were added tert-butyl 4-((4-(2-methoxy-4-(1,4,5-trimethyl-6-oxo-1,6-dihydropyridin-3-yl)benzyl)piperazin-1-yl)methyl)piperidine-1-carboxylate (1.089 mmol), TFA (1 ml, 12.98 mmol) and DCM (6 ml). The RM was stirred at RT for 1 h, then concentrated and the residue was purified by reversed phase chromatography on a REDISEP® Gold HP C18 column (15.5 g) eluting with ACN (from 2% to 10%) in an aq. solution of TFA (0.1%), yielding the corresponding TFA salt of the title compound as a solid (867 mg).

Method D: Rt=0.42 min; [M+H]⁺=439.

### Intermediate 20

### 4-(3-Methoxy-4-((1-(piperidin-4-ylmethyl)piperidin-4-yl)oxy)phenyl)-2-methyl-2,7-naphthyridin-1(2H)-one

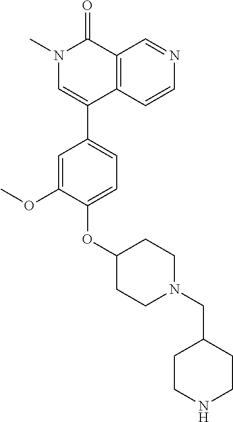 [SMILES: COc1cc(-c2cn(C)c(=O)c3cnccc23)ccc1OC1CCN(CC2CCNCC2)CC1]

### Step 1: tert-butyl 4-(2-methoxy-4-(2-methyl-1-oxo-1,2-dihydro-2,7-naphthyridin-4-yl)phenoxy_piperidine-1-carboxylate

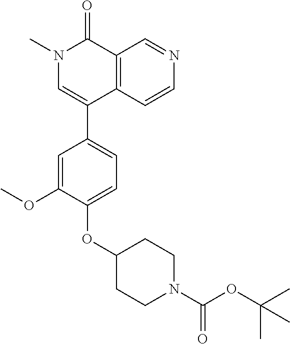 [SMILES: COc1cc(-c2cn(C)c(=O)c3cnccc23)ccc1OC1CCN(C(=O)OC(C)(C)C)CC1]

To a 250 ml round bottom flask were added under an argon atmosphere 4-bromo-2-methyl-2,7-naphthyridin-1(2H)-one (intermediate 5, 2.3 g, 7.02 mmol), tert-butyl 4-(2-methoxy-4-(4,4,5,5-tetramethyl-1,3,2-dioxaborolan-2-yl)phenoxy)piperidine-1-carboxylate (intermediate 16, 3.5 g, 8.08 mmol), K₂CO₃ (3.0 g, 21.71 mmol), ACN (40 ml) and water (10 ml). Solid PdCl₂(dppf) (500 mg, 0.683 mmol) was added and the RM was stirred at 100° C. for 2.5 h. The mixture was cooled to RT, filtered through CELITE®, the filtrate was diluted with Et₂O and the resulting mixture was filtered, yielding the title compound as a solid (3.4 g).

Method D: Rt=1.06 min; [M+H]⁺=466.

### Step 2: 4-(3-methoxy-4-(piperidin-4-yloxy)phenyl)-2-methyl-2,7-naphthyridin-1(2H)-one

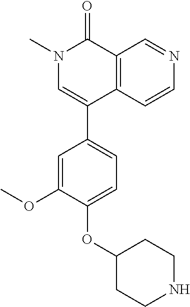 [SMILES: COc1cc(-c2cn(C)c(=O)c3cnccc23)ccc1OC1CCNCC1]

To a 25 ml round bottom flask were added tert-butyl 4-(2-methoxy-4-(2-methyl-1-oxo-1,2-dihydro-2,7-naphthyridin-4-yl)phenoxy)piperidine-1-carboxylate (1.0 g, 1.89 mmol), TFA (5 ml, 64.9 mmol) and DCM (5 ml). The RM was stirred at RT for 1 h, concentrated and the residue was purified by reversed phase chromatography on a REDISEP® Gold HP C18 column (50 g) eluting with ACN (from 2% to 100%) in an aq. solution of TFA (0.1%), yielding the corresponding TFA salt of the title compound as a solid (379 mg).

Method D: Rt=0.50 min; [M+H]⁺=366.

### Step 3: tert-butyl 4-((4-(2-methoxy-4-(2-methyl-1-oxo-1,2-dihydro-2,7-naphthyridin-4-yl)phenoxy)piperidin-1-yl)methyl)piperidine-1-carboxylate

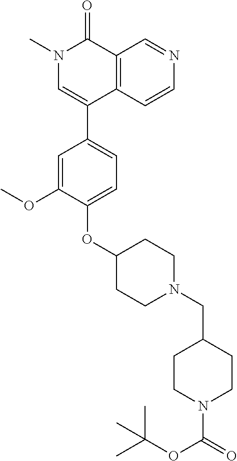 [SMILES: COc1cc(-c2cn(C)c(=O)c3cnccc23)ccc1OC1CCN(CC2CCN(C(=O)OC(C)(C)C)CC2)CC1]

To a 10 ml round bottom flask was added 4-(3-methoxy-4-(piperidin-4-yloxy)phenyl)-2-methyl-2,7-naphthyridin-1(2H)-one TFA salt (121 mg, 0.161 mmol), tert-butyl 4-formylpiperidine-1-carboxylate (42 mg, 0.197 mmol), TEA (0.100 ml, 0.717 mmol), a solution of ZnCl₂ (0.5 M) in THE (0.350 ml, 0.175 mmol) and MeOH (1.5 ml). The RM was stirred at RT for 7 h, then solid NaBH₃CN (11 mg, 0.175 mmol) was added. The RM was stirred at RT for 4 days, then concentrated and the residue was purified by reversed phase chromatography on a REDISEP® Gold HP C18 column (15.5 g) eluting with ACN (from 2% to 100%) in an aq. solution of TFA (0.1%), yielding the corresponding TFA salt of the title compound as a solid (130 mg).

Method D: Rt=0.77 min; [M+H]⁺=563.

### Step 4: 4-(3-methoxy-4-((1-(piperidin-4-ylmethyl)piperidin-4-yl)oxy)phenyl)-2-methyl-2,7-naphthyridin-1(2H)-one

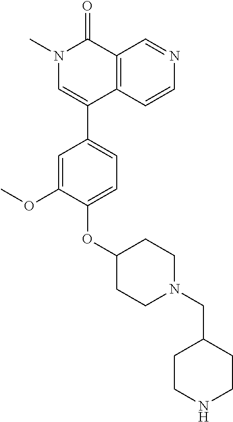 [SMILES: COc1cc(-c2cn(C)c(=O)c3cnccc23)ccc1OC1CCN(CC2CCNCC2)CC1]

To a 25 ml round bottom flask were added tert-butyl 4-((4-(2-methoxy-4-(2-methyl-1-oxo-1,2-dihydro-2,7-naphthyridin-4-yl)phenoxy)piperidin-1-yl)methyl)piperidine-1-carboxylate TFA salt (130 mg, 0.164 mmol), TFA (0.5 ml, 6.49 mmol) and DCM (2 ml). The RM was stirred at RT for 1 h, then concentrated and the residue was purified by reversed phase chromatography on a REDISEP® Gold HP C18 column (15.5 g) eluting with ACN (from 2% to 100%) in an aq. solution of TFA (0.1%), yielding the corresponding TFA salt of the title compound as a solid (64 mg).

Method D: Rt=0.50 min; [M+H]⁺=463.

### Intermediate 21

### 4-(3,5-Dimethoxy-4-((1-(piperidin-4-ylmethyl)piperidin-4-yl)oxy)phenyl)-2-methyl-2,7-naphthyridin-1(2H)-one

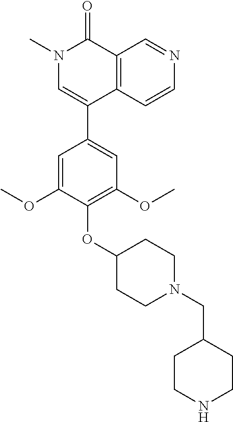 [SMILES: COc1cc(-c2cn(C)c(=O)c3cnccc23)cc(OC)c1OC1CCN(CC2CCNCC2)CC1]

### Step 1: tert-butyl 4-(2,6-dimethoxy-4-(2-methyl-1-oxo-1,2-dihydro-2,7-naphthyridin-4-yl)phenoxy)piperidine-1-carboxylate

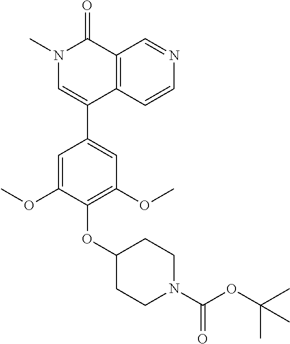 [SMILES: COc1cc(-c2cn(C)c(=O)c3cnccc23)cc(OC)c1OC1CCN(C(=O)OC(C)(C)C)CC1]

To a 25 ml round bottom flask were added under an argon atmosphere tert-butyl 4-(2,6-dimethoxy-4-(4,4,5,5-tetramethyl-1,3,2-dioxaborolan-2-yl)phenoxy)piperidine-1-carboxylate (intermediate 17, 398 mg, 0.773 mmol), 4-bromo-2-methyl-2,7-naphthyridin-1(2H)-one (intermediate 5, 253 mg, 0.773 mmol), K₂CO₃ (321 mg, 2.319 mmol), ACN (6 ml) and water (1.5 ml). Solid PdCl₂(dppf)(56 mg, 0.077 mmol) was added and the RM was stirred at 100° C. for 1.5 h. The mixture was cooled to RT, filtered through CELITE®, the filtrate was concentrated and the residue was titrated in Et₂O. The mixture was filtered and the solids were dried, yielding the title compound as a solid (400 mg), which was directly used for the next step without further purification.

Method D: Rt=1.08 min; [M+H]⁺=496.

### Step 2: 4-(3,5-dimethoxy-4-(piperidin-4-yloxy)phenyl)-2-methyl-2,7-naphthyridin-1(2H)-one

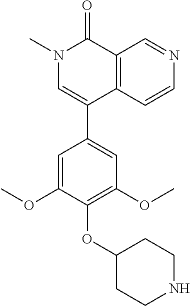

To a 25 ml round bottom flask were added tert-butyl 4-(2,6-dimethoxy-4-(2-methyl-1-oxo-1,2-dihydro-2,7-naphthyridin-4-yl)phenoxy)piperidine-1-carboxylate (400 mg, 0.767 mmol), TFA (2 ml, 26.0 mmol) and DCM (1 ml). The RM was stirred at RT for 1 h, then concentrated and the residue was purified by reversed phase chromatography on a REDISEP® Gold HP C18 column (50 g) eluting with ACN (from 2% to 100%) in an aq. solution of TFA (0.1%), yielding the corresponding TFA salt of the title compound as a solid (213 mg).

Method D: Rt=0.53 min; [M+H]⁺=396.

### Step 3: tert-butyl 4-((4-(2,6-dimethoxy-4-(2-methyl-1-oxo-1,2-dihydro-2,7-naphthyridin-4-yl)phenoxy)piperidin-1-yl)methyl)piperidine-1-carboxylate

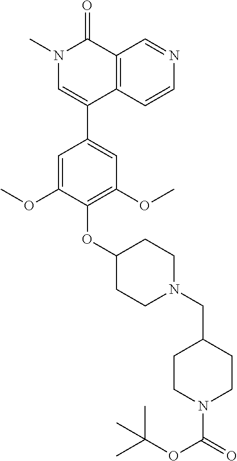 [SMILES: COc1cc(-c2cn(C)c(=O)c3cnccc23)cc(OC)c1OC1CCN(CC2CCN(C(=O)OC(C)(C)C)CC2)CC1]

To a 10 ml round bottom flask were added 4-(3,5-dimethoxy-4-(piperidin-4-yloxy)phenyl)-2-methyl-2,7-naphthyridin-1(2H)-one TFA salt (100 mg, 0.194 mmol), tert-butyl 4-formylpiperidine-1-carboxylate (50 mg, 0.234 mmol), TEA (0.100 ml, 0.717 mmol), a solution of ZnCl₂ (0.5 M) in THF (0.450 ml, 0.225 mmol) and MeOH (1.5 ml). The RM was stirred at RT for 7 h. Solid NaBH₃CN (14 mg, 0.223 mmol) was added and the RM was stirred at RT for 2 days. The mixture was concentrated and the residue purified by reversed phase chromatography on a REDISEP® Gold HP C18 column (15.5 g) eluting with ACN (from 2% to 100%) in an aq. solution of TFA (0.1%), yielding the corresponding TFA salt of the title compound as a solid (145 mg).

Method D: Rt=0.81 min; [M+H]⁺=577.

### Step 4: 4-(3,5-dimethoxy-4-((1-(piperidin-4-ylmethyl)piperidin-4-yl)oxy)phenyl)-2-methyl-2,7-naphthyridin-1(2H)-one

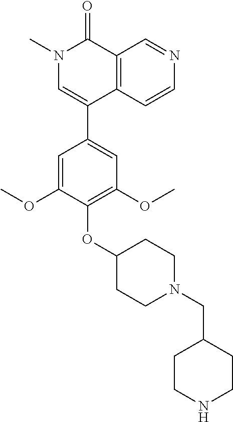 [SMILES: COc1cc(-c2cn(C)c(=O)c3cnccc23)cc(OC)c1OC1CCN(CC2CCNCC2)CC1]

To a 10 ml round bottom flask were added tert-butyl 4-((4-(2,6-dimethoxy-4-(2-methyl-1-oxo-1,2-dihydro-2,7-naphthyridin-4-yl)phenoxy)piperidin-1-yl)methyl)piperidine-1-carboxylate TFA salt (141 mg, 0.190 mmol), TFA (0.500 ml, 6.49 mmol) and DCM (1 ml). The RM was stirred at RT for 1 h, then the mixture was concentrated and the residue was dissolved in a mixture of water and ACN and freeze dried, yielding the corresponding TFA salt of the title compound as a solid (130 mg).

Method D: Rt=0.42 min; [M+H]⁺=493.

### Intermediate 22

### 3-(2,4-Dioxotetrahydropyrimidin-1(2H)-yl)benzoic Acid

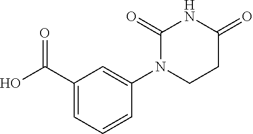 [SMILES: O=C1CCN(c2cccc(C(=O)O)c2)C(=O)N1]

### Step 1: 3-((2-carboxyethyl)amino)benzoic Acid

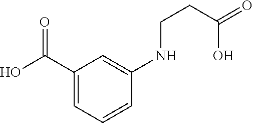 [SMILES: O=C(O)CCNc1cccc(C(=O)O)c1]

To a 500 ml round bottom flask were added 3-aminobenzoic acid (10 g, 72.92 mmol), acrylic acid (6.83 g, 94.79 mmol) and toluene (200 ml). The RM was stirred at 120° C. for 48 h, filtered, the solids were washed with toluene (2×2 ml) and dried to afford the title compound as a solid (14 g).

Method A: Rt=1.20 min; [M+H]⁺=210.

### Step 2: 3-(2,4-dioxotetrahydropyrimidin-1(2H)-yl)benzoic Acid

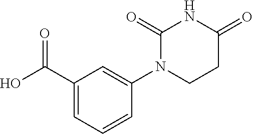 [SMILES: O=C1CCN(c2cccc(C(=O)O)c2)C(=O)N1]

To a 50 ml round bottom flask were added 3-((2-carboxyethyl)amino)benzoic acid (2 g, 9.56 mmol), urea (1.72 g, 28.68 mmol) and HOAc (25 ml). The RM was stirred at 120° C. for 16 h. The mixture was concentrated, water (20 ml) was added, cooled to 0° C., filtered and the solids were washed with cold water (2×5 ml) and dried. The solids were dispersed in DMF (10 ml), the mixture was stirred at RT for 2 h, filtered and the solids were washed with cold water (2×5 ml) and dried to afford the title compound as a solid (800 mg).

Method A: Rt=1.13 min; [M+H]⁺=235.

¹H NMR (500 MHz, DMSO-d₆) δ 13.12 (s, 1H), 10.46 (s, 1H), 7.89 (s, 1H), 7.80 (d, J=7.6 Hz, 1H), 7.59 (d, J=8.6 Hz, 1H), 7.52 (t, J=7.8 Hz, 1H), 3.84 (t, J=6.6 Hz, 2H), 2.73 (t, J=6.6 Hz, 2H).

### Intermediate 23

### 3-(2,4-Dioxotetrahydropyrimidin-1(2H)-yl)-4-methylbenzoic Acid

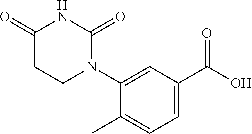 [SMILES: Cc1ccc(C(=O)O)cc1N1CCC(=O)NC1=O]

### Step 1: 3-((2-carboxyethyl)amino)-4-methylbenzoic Acid

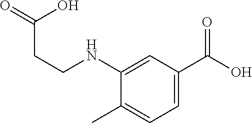 [SMILES: Cc1ccc(C(=O)O)cc1NCCC(=O)O]

To a 500 ml round bottom flask was added 3-amino-4-methylbenzoic acid (50 g, 331 mmol), acrylic acid (95 g, 1320 mmol) and toluene (100 ml). The RM was stirred at 100° C. for 2 h, concentrated and directly used for the next step without further purification.

Method E: Rt=1.36 min; [M+H]⁺=224.

### Step 2: 3-(2,4-dioxotetrahydropyrimidin-1(2H)-yl)-4-methylbenzoic Acid

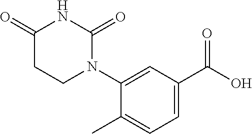 [SMILES: Cc1ccc(C(=O)O)cc1N1CCC(=O)NC1=O]

To a 1 L round bottom flask was added 3-((2-carboxyethyl)amino)-4-methylbenzoic acid (62 g, 278 mmol), urea (100 g, 1670 mmol) and acetic acid (500 ml). The RM was stirred at 120° C. for 18 h, cooled to RT and added onto crashed ice (800 g). An aq. solution of HCl (1 M, 400 ml) was added, the mixture was stirred at RT for 2 h, filtered and the solids were washed with cold ACN (500 ml), yielding the title as a solid (62 g).

Method E: Rt=1.24 min; [M+H]⁺=249.

### Intermediate 24

### 4-Chloro-3-(2,4-dioxotetrahydropyrimidin-1(2H)-yl)benzoic Acid

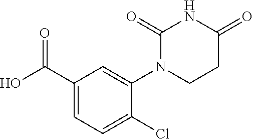 [SMILES: O=C1CCN(c2cc(C(=O)O)ccc2Cl)C(=O)N1]

### Step 1: 3-((2-chloro-5-(methoxycarbonyl)phenyl)amino)propanoic Acid

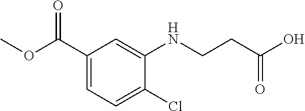 [SMILES: COC(=O)c1ccc(Cl)c(NCCC(=O)O)c1]

To a 250 ml round bottom flask were added methyl 3-amino-4-chlorobenzoate (20 g, 108 mmol) and acrylic acid (31 g, 432 mmol). The RM was stirred at 100° C. for 16 h, added into water (50 ml) and extracted with EtOAc (500 ml). The organic phase was washed with water (50 ml), brine (50 ml) and dried over Na₂SO₄, yielding the title compound as a solid (35 g).

Method H: Rt=1.21 min; [M+H]⁺=258.

### Step 2: methyl 4-chloro-3-(2,4-dioxotetrahydropyrimidin-1(2H)-yl)benzoate

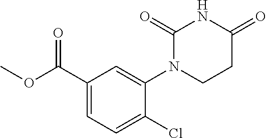 [SMILES: COC(=O)c1ccc(Cl)c(N2CCC(=O)NC2=O)c1]

To a 1 L round bottom flask were added 3-((2-chloro-5-(methoxycarbonyl)phenyl)amino)propanoic acid (35 g, 108 mmol), urea (54.4 g, 756 mmol) and acetic acid (300 ml). The RM was stirred at 110° C. for 18 h, added into water (200 ml) and extracted with EtOAc (2×200 ml). The combined organic phases were washed with water (100 ml), brine (100 ml) and dried over Na₂SO₄. MTBE (60 mL) was added to the residue, the mixture was filtered and the solids were washed with cold MTBE (50 mL), yielding the title compound as a solid (9.6 g).

Method H: Rt=1.46 min; [M+H]⁺=283.

### Step 3: 4-chloro-3-(2,4-dioxotetrahydropyrimidin-1(2H)-yl)benzoic Acid

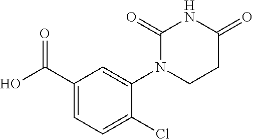 [SMILES: O=C1CCN(c2cc(C(=O)O)ccc2Cl)C(=O)N1]

To a 100 ml round bottom flask were added methyl 4-chloro-3-(2,4-dioxotetrahydropyrimidin-1(2H)-yl)benzoate (5.80 g, 18.35 mmol), an aq. solution of HCl (6 M, 30 ml) and 1,4-dioxane (20 ml). The RM was stirred at 90° C. for 66 h. The mixture was concentrated, diluted with MTBE (10 ml), filtered and the solids were washed with cold MTBE (5 ml), an aq. solution of HCl (0.01 M, 5 ml), cold ACN (5 ml) and dried to afford the title compound as a solid (4.90 g).

Method E: Rt=1.30 min; [M+H]⁺=269.

¹H NMR (500 MHz, DMSO-d₆) δ 13.34 (s, 1H), 10.52 (s, 1H), 8.04 (d, J=2.0 Hz, 1H), 7.91 (dd, J=8.4, 2.1 Hz, 1H), 7.71 (d, J=8.4 Hz, 1H), 3.82-3.73 (m, 1H), 3.66-3.58 (m, 1H), 2.84-2.67 (m, 2H).

### Intermediate 25

### 3-(2,4-Dioxotetrahydropyrimidin-1(2H)-yl)-4-methoxybenzoic Acid

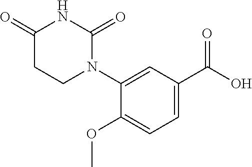 [SMILES: COc1ccc(C(=O)O)cc1N1CCC(=O)NC1=O]

### Step 1: 3-((2-carboxyethyl)amino)-4-methoxybenzoic Acid

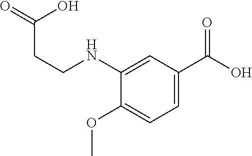 [SMILES: COc1ccc(C(=O)O)cc1NCCC(=O)O]

To a 250 ml round bottom flask were added 3-amino-4-methoxybenzoic acid (35 g, 209 mmol), acrylic acid (60 g, 836 mmol) and toluene (130 ml). The RM was stirred at 100° C. for 16 h. MTBE (50 ml) was added and the mixture was stirred at 0° C. for 30 min, filtered and the solids were washed with PE, yielding the title compound as a solid (46 g).

Method E: Rt=1.31 min; [M+H]⁺=240.

### Step 2: 3-(2,4-dioxotetrahydrophyrimidin-1(2H)-yl)-4-methoxybenzoic Acid

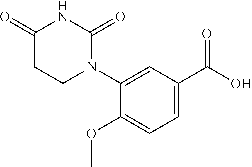 [SMILES: COc1ccc(C(=O)O)cc1N1CCC(=O)NC1=O]

To a 1 L round bottom flask were added 3-((2-carboxyethyl)amino)-4-methoxybenzoic acid (46 g, 192 mmol), urea (92 g, 1533 mmol) and acetic acid (230 ml). The RM was stirred at 120° C. for 18 h, cooled to RT, added into an aq. solution of HCl (0.5 M, 800 ml) and the mixture was stirred at RT for 1 h. The mixture was filtered and the solids were washed with cold water (500 ml), yielding the title compound as a solid (36.2 g).

Method E: Rt=1.23 min; [M+H]⁺=265.

### Intermediate 26

### Perfluorophenyl 3-(2,4-dioxotetrahydropyrimidin-1(2H)-yl)-4-methoxybenzoate

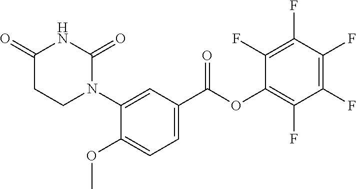 [SMILES: COc1ccc(C(=O)Oc2c(F)c(F)c(F)c(F)c2F)cc1N1CCC(=O)NC1=O]

To a 2 L round bottom flask were added 3-(2,4-dioxotetrahydropyrimidin-1(2H)-yl)-4-methoxybenzoic acid (intermediate 25, 50 g, 189 mmol), perfluorophenyl 2,2,2-trifluoroacetate (63.5 g, 227 mmol) and DMF (125 ml). The RM was stirred at 0° C. for 10 min, DIEA (80 ml, 450 mmol) was added dropwise over 30 min, the RM was allowed to reach RT and stirring was continued for additional 2 h. The mixture was added into EtOAc (1 L), the organic phase was washed with brine (5×125 ml) and the residue was purified by chromatography on silica gel eluting with EA (from 0% to 70%) in PE, yielding the title compound as a solid (68.78 g).

Method H: Rt=2.01 min; [M+H]⁺=431.

### Intermediate 27

### 1-(3-(4-(Piperidin-4-yloxy)piperidine-1-carbonyl)phenyl)dihydropyrimidine-2,4(1H,3H)-dione

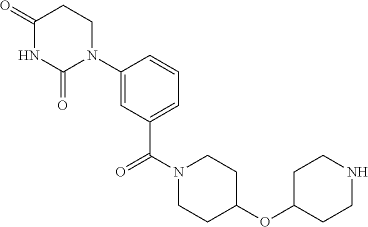 [SMILES: O=C1CCN(c2cccc(C(=O)N3CCC(OC4CCNCC4)CC3)c2)C(=O)N1]

### Step 1: tert-butyl 4-((1-(3-(2,4-dioxotetrahydropyrimidin-1(2H)-yl)benzoyl)piperidin-4-yl)oxy)piperidine-1-carboxylate

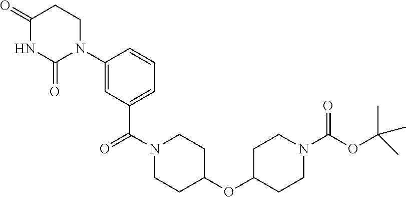 [SMILES: CC(C)(C)OC(=O)N1CCC(OC2CCN(C(=O)c3cccc(N4CCC(=O)NC4=O)c3)CC2)CC1]

To a 100 ml round bottom flask were added 3-(2,4-dioxotetrahydropyrimidin-1(2H)-yl)benzoic acid (intermediate 22, 247 mg, 1.05 mmol), tert-butyl 4-(piperidin-4-yloxy)piperidine-1-carboxylate (intermediate 1, 300 mg, 1.05 mmol), TEA (0.6 ml, 4.2 mmol), HATU (478 mg, 1.26 mmol) and DMF (10 ml). The RM was stirred at RT for 1 h, then EtOAc (60 ml) was added and the organic phase was washed with brine (3×20 ml), dried over Na₂SO₄ and the solid residue (0.5 g), containing the title compound, was directly used for the next step without further purification.

Method I: Rt=1.65 min; [M+Na]⁺=523.

### Step 2:1-(3-(4-(piperidin-4-yloxy)piperidine-1-carbonyl)phenyl)dihydropyrimidine-2,4(1H,3H)-dione

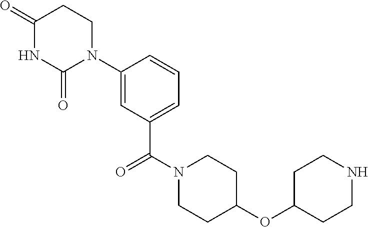 [SMILES: O=C1CCN(c2cccc(C(=O)N3CCC(OC4CCNCC4)CC3)c2)C(=O)N1]

To a 100 ml round bottom flask were added tert-butyl 4-((1-(3-(2,4-dioxotetrahydropyrimidin-1(2H)-yl)benzoyl)piperidin-4-yl)oxy)piperidine-1-carboxylate (500 mg, 0.89 mmol), a solution of HCl (4 M) in 1,4-dioxane (10 ml) and DCM (20 ml) and the RM was stirred at RT for 2 h. The mixture was concentrated to dryness, yielding the title compound as the corresponding hydrochloride salt (550 mg), which was directly used for the next step without further purification.

Method J: Rt=0.76 min; [M+H]⁺=401.

### Intermediate 28

### 1-(2-Chloro-5-(4-(piperidin-4-yloxy)piperidine-1-carbonyl)phenyl)dihydropyrimidine-2,4(1H,3H)-dione

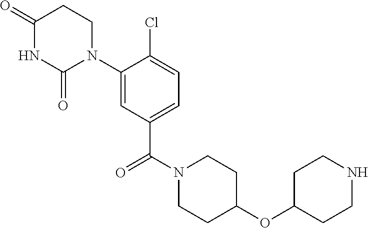 [SMILES: O=C1CCN(c2cc(C(=O)N3CCC(OC4CCNCC4)CC3)ccc2Cl)C(=O)N1]

### Step 1: tert-butyl 4-((1-(4-chloro-3-(2,4-dioxotetrahydropyrimidin-1(2H)-yl)benzoyl)piperidin-4-yl)oxy)piperidine-1-carboxylate

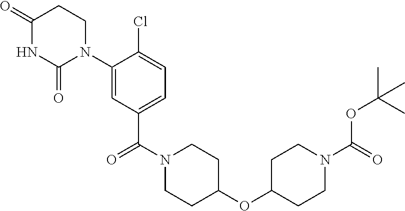 [SMILES: CC(C)(C)OC(=O)N1CCC(OC2CCN(C(=O)c3ccc(Cl)c(N4CCC(=O)NC4=O)c3)CC2)CC1]

To a 50 ml round bottom flask were added HATU (849 mg, 2.233 mmol), 4-chloro-3-(2,4-dioxotetrahydropyrimidin-1(2H)-yl)benzoic acid (intermediate 24, 500 mg, 1.861 mmol), DIPEA (1 ml, 5.73 mmol) and DMF (10 ml). The RM was stirred at RT for 30 min, solid tert-butyl 4-(piperidin-4-yloxy)piperidine-1-carboxylate (intermediate 1, 529 mg, 1.861 mmol) was added and the RM was stirred at RT for 1.5 h. The solvent was removed and the residue was purified by reversed phase chromatography on a REDISEP® Gold HP C18 column (50 g) eluting with ACN (from 2% to 100%) in an aq. solution of TFA (0.1%), yielding the title compound as a solid (1.07 g).

Method D: Rt=0.98 min; [M+H]⁺=535.

### Step 2: 1-(2-chloro-5-(4-(piperidin-4-yloxy)piperidine-1-carbonyl)phenyl)dihydropyrimidine-2,4(1H,3H)-dione

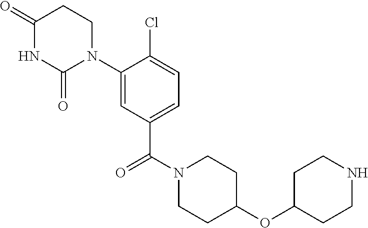 [SMILES: O=C1CCN(c2cc(C(=O)N3CCC(OC4CCNCC4)CC3)ccc2Cl)C(=O)N1]

To a 25 ml round bottom flask were added tert-butyl 4-((1-(4-chloro-3-(2,4-dioxotetrahydropyrimidin-1(2H)-yl)benzoyl)piperidin-4-yl)oxy)piperidine-1-carboxylate (1.07 g, 1.820 mmol), a solution of HCl (4 M) in 1,4-dioxane (9 ml) and 1,4-dioxane (9 ml). The RM was stirred at RT for 3 h, the solvents were removed, the residue was redissolved in a mixture of water and ACN and freeze dried, yielding the corresponding hydrochloride salt of the title compound as a solid (884 mg).

Method D: Rt=0.47 min; [M+H]⁺=435.

### Intermediate 29

### 1-(2-Methoxy-5-(4-(piperidin-4-yloxy)piperidine-1-carbonyl)phenyl)dihydropyrimidine-2,4(1H,3H)-dione

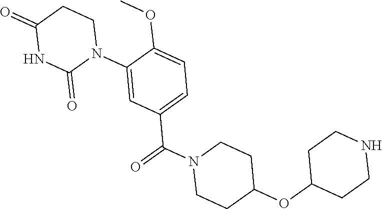 [SMILES: COc1ccc(C(=O)N2CCC(OC3CCNCC3)CC2)cc1N1CCC(=O)NC1=O]

### Step 1: tert-butyl 4-((1-(3-(2,4-dioxotetrahydropyrimidin-1(2H)-yl)-4-methoxybenzoyl)piperidin-4-yl)oxy)piperidine-1-carboxylate

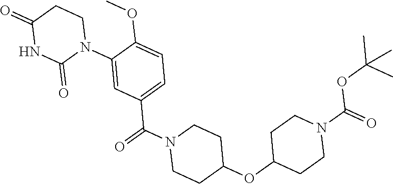 [SMILES: COc1ccc(C(=O)N2CCC(OC3CCN(C(=O)OC(C)(C)C)CC3)CC2)cc1N1CCC(=O)NC1=O]

To a 50 ml round bottom flask were added HATU (691 mg, 1.817 mmol), 3-(2,4-dioxotetrahydropyrimidin-1(2H)-yl)-4-methoxybenzoic acid (intermediate 25, 400 mg, 1.514 mmol), DIPEA (0.800 ml, 4.58 mmol) and DMF (10 ml). The RM was stirred at RT for 30 min, tert-butyl 4-(piperidin-4-yloxy)piperidine-1-carboxylate (intermediate 1, 431 mg, 1.514 mmol) was added and the RM was stirred at RT overnight. The mixture was directly purified by reversed phase chromatography on a REDISEP® Gold HP C18 column (50 g) eluting with ACN (from 0% to 100%) in an aq. solution of NH₄HCO₃ (0.1%), yielding the title compound as a solid (686 mg).

Method D: Rt=0.92 min; [M+H]⁺=531.

### Step 2: 1-(2-methoxy-5-(4-(piperidin-4-yloxy)piperidine-1-carbonyl)phenyl)dihydropyrimidine-2,4(1H,3H)-dione

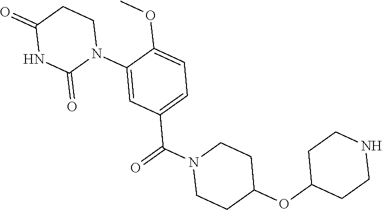 [SMILES: COc1ccc(C(=O)N2CCC(OC3CCNCC3)CC2)cc1N1CCC(=O)NC1=O]

To a 25 ml round bottom flask were added tert-butyl 4-((1-(3-(2,4-dioxotetrahydropyrimidin-1(2H)-yl)-4-methoxybenzoyl)piperidin-4-yl)oxy)piperidine-1-carboxylate (680 mg, 1.282 mmol), a solution of HCl (4 M) in 1,4-dioxane (8 ml) and 1,4-dioxane (8 ml). The RM was stirred at RT for 3 h, concentrated, the residue was diluted in water and ACN and freeze dried, yielding the corresponding hydrochloride salt of the title compound as a solid (655 mg).

Method K: Rt=0.80 min; [M+H]⁺=431.

### Intermediate 30

### 1-(4-(2-Oxo-2-(4-(piperidin-4-yloxy)piperidin-1-yl)ethoxy)phenyl)dihydropyrimidine-2,4(1H,3H)-dione

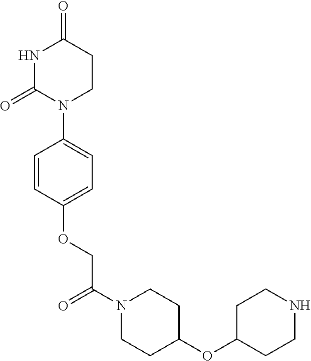 [SMILES: O=C1CCN(c2ccc(OCC(=O)N3CCC(OC4CCNCC4)CC3)cc2)C(=O)N1]

### Step 1: methyl 2-(4-((tert-butoxycarbonyl)amino)phenoxy)acetate

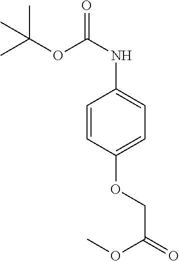 [SMILES: COC(=O)COc1ccc(NC(=O)OC(C)(C)C)cc1]

To a 250 ml round bottom flask were added tert-butyl (4-hydroxyphenyl)carbamate (7 g, 31.8 mmol), Cs₂CO₃ (11.4 g, 35.0 mmol), KI (5 mg, 0.301 mmol) and acetone (75 ml). Methyl bromoacetate was added and the RM was stirred at 70° C. for 4 h. The RM was cooled to RT, filtered and the filtrate was concentrated. The residue was diluted with EtOAc, washed with a sat. aq. solution of NaHCO₃, dried over MgSO₄ and evaporated. The residue was purified by chromatography on silica gel eluting with EtOAc (from 0% to 25%) in CHX, yielding the title compound as a solid (8.83 g).

Method D: Rt=0.97 min; [M+H]⁺=282.

### Step 2: methyl 2-(4-aminophenoxy)acetate

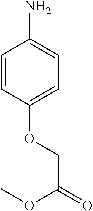

To a 100 ml round bottom flask were added methyl 2-(4-((tert-butoxycarbonyl)amino)phenoxy)acetate (8.83 g, 31.4 mmol), TFA (30 ml, 389 mmol) and 1,4-dioxane (30 ml). The RM was stirred at RT for 18 h and concentrated. The residue was diluted with DCM, the organic phase was washed with a sat. aq. solution of NaHCO₃ and dried over MgSO₄, yielding the title compound as an oil (5.35 g), which was directly used for next step without further purification.

Method D: Rt=0.37 min; [M+H]⁺=182.

### Step 3:3,3′-((4-(2-methoxy-2-oxoethoxy)phenyl)azanediyl)dipropionic Acid

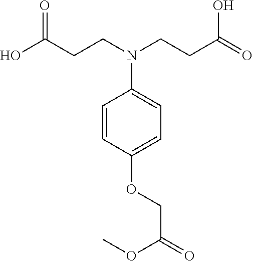 [SMILES: COC(=O)COc1ccc(N(CCC(=O)O)CCC(=O)O)cc1]

To a 100 ml round bottom flask were added methyl 2-(4-aminophenoxy)acetate (5.35 g, 25.7 mmol), acrylic acid (11 ml, 160 mmol) and water (5 ml). The RM was stirred at 70° C. for 1.5 h. The RM was cooled to RT, adsorbed on ISOLUTE® and purified by chromatography on silica gel eluting with a mixture (4:1) of DCM and iPrOH (from 0% to 50%) in DCM, yielding the title compound as a solid (8.24 g).

Method D: Rt=0.47 min; [M+H]⁺=326.

### Step 4: 2-(4-(2,4-dioxotetrahydropyrimidin-1(2H)-yl)phenoxy)acetic Acid

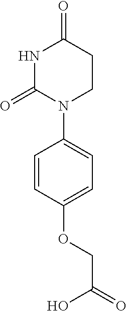 [SMILES: O=C(O)COc1ccc(N2CCC(=O)NC2=O)cc1]

To a 250 ml round bottom flask were added 3,3′-((4-(2-methoxy-2-oxoethoxy)phenyl)azanediyl)dipropionic acid (8.24 g, 25.09 mmol), urea (2.26 g, 37.6 mmol) and HOAc (60 ml). The RM was stirred at 120° C. overnight, an aq. solution of HCl (4 M, 80 ml) was added and the RM was stirred at 120° C. for 45 min. The RM was cooled to 0° C. and filtered, yielding the title compound as a solid (4.93 g).

Method D: Rt=0.75 min; [M+H]⁺=265.

### Step 5: tert-butyl 4-((1-(2-(4-(2,4-dioxotetrahydropyrimidin-1(2H)-yl)phenoxy)acetyl)piperidin-4-yl)oxy)piperidine-1-carboxylate

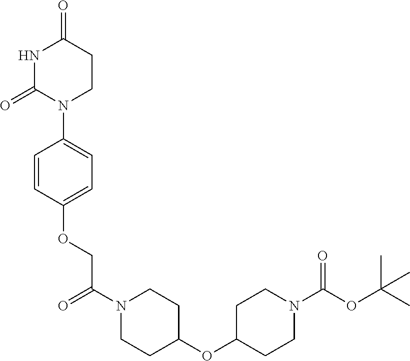 [SMILES: CC(C)(C)OC(=O)N1CCC(OC2CCN(C(=O)COc3ccc(N4CCC(=O)NC4=O)cc3)CC2)CC1]

To a 50 ml round bottom flask were added tert-butyl 4-(piperidin-4-yloxy)piperidine-1-carboxylate (intermediate 1, 538 mg, 1.892 mmol), 2-(4-(2,4-dioxotetrahydropyrimidin-1(2H)-yl)phenoxy)acetic acid (500 mg, 1.892 mmol), HATU (863 mg, 2.271 mmol), TEA (0.800 ml, 5.74 mmol) and DMF (8 ml). The RM was stirred at RT for 6 h. The mixture was diluted with EtOAc and water, the aqueous layer was extracted with EtOAc, the combined organic phases were washed with brine and dried over MgSO₄, yielding the title compound as a solid (795 mg), which was directly used for next step without further purification.

Method D: Rt=0.90 min; [M+H]⁺=531.

### Step 6: 1-(4-(2-oxo-2-(4-(piperidin-4-yloxy)piperidin-1-yl)ethoxy)phenyl)dihydropyrimidine-2,4(1H,3H)-dione

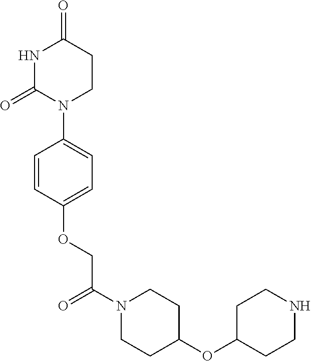 [SMILES: O=C1CCN(c2ccc(OCC(=O)N3CCC(OC4CCNCC4)CC3)cc2)C(=O)N1]

To a 25 ml round bottom flask were added tert-butyl 4-((1-(2-(4-(2,4-dioxotetrahydropyrimidin-1(2H)-yl)phenoxy)acetyl)piperidin-4-yl)oxy)piperidine-1-carboxylate (795 mg, 1.423 mmol), a solution of HCl (4 M) in 1,4-dioxane (10 ml, 40.0 mmol), MeOH (5 ml) and DCM (5 ml). The RM was stirred at RT for 1 h, concentrated, diluted with water and freeze dried, yielding the corresponding hydrochloride salt of the title compound as a solid (785 mg).

Method D: Rt=0.43 min; [M+H]⁺=431.

### Intermediate 31

### 5-bromo-3,4-dimethyl-1-propylpyridin-2(1H)-one

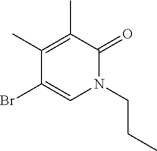

### Step 1: 5-bromo-3,4-dimethylpyridin-2(1H)-one

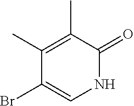

To a 500 ml round bottom flask were added 5-bromo-3,4-dimethylpyridin-2-amine (10 g, 49.7 mmol), water (322 ml) and conc. H₂SO₄ (27.7 ml). The RM was stirred at 0° C. for 5 min, solid NaNO₂ (4.12 g, 59.7 mmol) was added and the RM was allowed to warm to RT and stirring was continued at RT for 4 h. The mixture was filtered and the solids were washed with water (20 ml) and dried, yielding the title compound as a solid (9.0 g).

Method E: Rt=1.45 min; [M+H]⁺=202, 204.

### Step 2: 5-bromo-3,4-dimethyl-1-propylpyridin-2(1H)-one

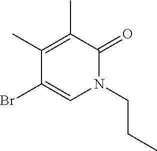

To a 100 ml round bottom flask was added 5-bromo-3,4-dimethylpyridin-2(1H)-one (2.5 g, 12.4 mmol) and DMF (15 ml). The mixture was cooled to 0° C., solid NaH (60% dispersion in mineral oil, 595 mg, 14.8 mmol) was added portionwise and stirring was continued for 5 min. Iodopropane (3.15 g, 22 mmol) was added dropwise over 5 min, the RM was stirred at RT for 16 h, then added into cold water (50 ml). The mixture was extracted with EtOAc (3×20 ml), the combined organic phases were washed with brine (20 ml), dried over Na₂SO₄ and the residue was purified by chromatography on silica gel eluting with EtOAc (from 5% to 10%) in PE, yielding the title compound as a solid (2.22 g).

Method E: Rt=1.76 min; [M+H]⁺=244, 246.

### Intermediate 32

### 4-bromo-2-butyl-2,7-naphthyridin-1(2H)-one

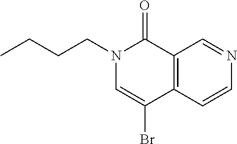 [SMILES: CCCCn1cc(Br)c2ccncc2c1=O]

To a 500 ml round bottom flask were added 4-bromo-2,7-naphthyridin-1(2H)-one (4 g, 17.24 mmol) and DMF (100 ml). The mixture was cooled to 0° C. and solid NaH (60% in mineral oil, 1 g, 25.00 mmol) was added portionwise and the mixture was stirred at 0° C. for 30 min. Iodobutane (3 ml, 33.80 mmol) was slowly added and the RM was stirred at RT overnight. Water was added and the mixture was extracted with EtOAc. The combined organic phases were washed with brine and dried over MgSO₄, yielding the title compound as a solid (5.5 g), which was directly used for the next step without further purification.

Method D: Rt=0.97 min; [M+H]⁺=281, 283.

### Intermediate 33

### 4-(2-butyl-1-oxo-1,2-dihydro-2,7-naphthyridin-4-yl)-2,5-dimethoxybenzaldehyde

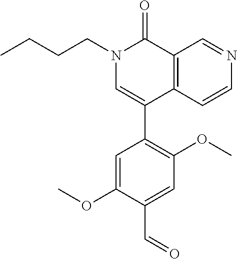 [SMILES: CCCCn1cc(-c2cc(OC)c(C=O)cc2OC)c2ccncc2c1=O]

### Step 1: 2,5-dimethoxy-4-(4,4,5,5-tetramethyl-1,3,2-dioxaborolan-2-yl)benzaldehyde

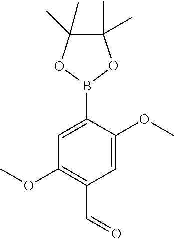

To a 100 ml round bottom flask were added under an argon atmosphere 4-bromo-2,5-dimethoxybenzaldehyde (5.00 g, 19.38 mmol), BISPIN (8.12 g, 32.00 mmol), KOAc (5.71 mg, 58.10 mmol) and 1,4-dioxane (50 ml). Solid PdCl₂(dppf)-CH₂Cl₂ (475 mg, 0.581 mmol) was added and the RM was stirred at 100° C. for 18 h. The RM was allowed to cool to RT, filtered over CELITE® and the solids were washed with EtOAc. The filtrate was washed with an aq. solution of HCl (0.1 M) and brine, the organic phase was dried over MgSO₄ and the residue was purified by chromatography on silica gel eluting with EtOAc (from 0% to 50%) in CHX, yielding the title compound as a solid (5.52 g).

Method M: Rt=0.74 min; [M+H]⁺=211.

### Step 2: 4-(2-butyl-1-oxo-1,2-dihydro-2,7-naphthyridin-4-yl)-2,5-dimethoxybenzaldehyde

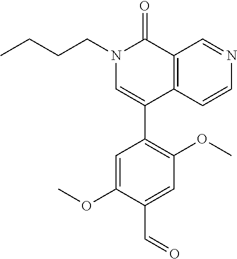 [SMILES: CCCCn1cc(-c2cc(OC)c(C=O)cc2OC)c2ccncc2c1=O]

To a 50 ml round bottom flask were added under an argon atmosphere 2,5-dimethoxy-4-(4,4,5,5-tetramethyl-1,3,2-dioxaborolan-2-yl)benzaldehyde (625 mg, 2.054 mmol), 4-bromo-2-butyl-2,7-naphthyridin-1(2H)-one (intermediate 32, 500 mg, 1.369 mmol), Na₂CO₃ (435 mg, 4.11 mmol), water (2 ml) and 1,4-dioxane (8 ml). Solid PdCl₂(dppf)-CH₂Cl₂ (51 mg, 0.069 mmol) was added and the RM was stirred at 100° C. for 1 h. The RM was allowed to cool to RT, filtered over CELITE®, the filtrate was diluted with water and the mixture was extracted with EtOAc. The combined organic phases were dried over MgSO₄ and the residue was purified by chromatography on silica gel eluting with EtOAc (from 0% to 100%) in CHX and then with MeOH (from 0% to 10%) in EtOAc, yielding the title compound as a solid (498 mg).

Method M: Rt=0.96 min; [M+H]⁺=367.

### Intermediate 34

### tert-butyl 4-((1-(2,6-dimethoxy-4-(4,4,5,5-tetramethyl-1,3,2-dioxaborolan-2-yl)phenethyl)piperidin-4-yl)oxy)piperidine-1-carboxylate

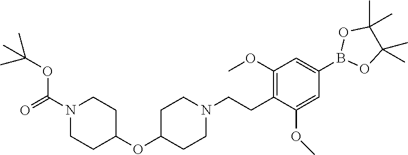 [SMILES: COc1cc(B2OC(C)(C)C(C)(C)O2)cc(OC)c1CCN1CCC(OC2CCN(C(=O)OC(C)(C)C)CC2)CC1]

### Step 1: 5-bromo-1,3-dimethoxy-2-(2-methoxyvinyl)benzene

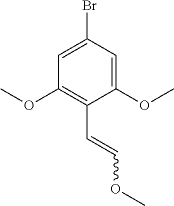

To a 100 ml round bottom flask were added (methoxymethyl)triphenylphosphonium chloride (21 g, 20.5 mmol), t-BuOK (9.2 g, 82 mmol) and THE (100 ml). The RM was stirred at 0° C. for 30 min and 4-bromo-2,6-dimethoxybenzaldehyde (5 g, 20.5 mmol) was added. The RM was stirred at 0° C. for 1 h, then at 70° C. for 16 h. The mixture was added into water (100 ml), extracted with EtOAc (2×100 ml), the combined organic phases were washed with brine (2×50 mL), dried over Na₂SO₄ and the residue was purified by chromatography on silica gel eluting with EtOAc (from 20% to 50%) in PE, yielding the title compound as a solid (2.3 g).

Method H: Rt=2.11 min; [M+H]⁺=273, 275.

### Step 2: 2-(4-bromo-2,6-dimethoxyphenyl)acetaldehyde

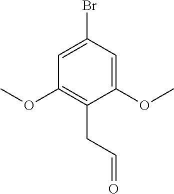

To a 250 ml round bottom flask were added 5-bromo-1,3-dimethoxy-2-(2-methoxyvinyl)benzene (2.3 g, 8.46 mmol), acetone (40 ml) and an aq. solution of HCl (2 M, 4 ml). The RM was stirred at 65° C. for 3 h then concentrated to afford the title compound as an oil (2.3 g), which was directly used for the next step without further purification.

Method H: Rt=2.08 min; [M+H]⁺=259, 261.

### Step 3: tert-butyl 4-((1-(4-bromo-2,6-dimethoxyphenethyl)piperidin-4-yl)oxy)piperidine-1-carboxylate

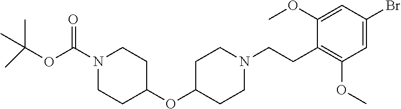 [SMILES: COc1cc(Br)cc(OC)c1CCN1CCC(OC2CCN(C(=O)OC(C)(C)C)CC2)CC1]

To a 250 ml round bottom flask were added 2-(4-bromo-2,6-dimethoxyphenyl)acetaldehyde (2.3 g, 8.88 mmol), tert-butyl 4-(piperidin-4-yloxy)piperidine-1-carboxylate (intermediate 1, 3.26 g, 10.65 mmol), a solution of ZnCl₂ (1 M) in THF (12 ml, 12 mmol) and DMSO (30 ml). The RM was stirred at RT for 1 h and solid NaBH₃CN (1.12 g, 17.76 mmol) was added. The RM was stirred at RT for 16 h, the solvent was removed and the residue was purified by reversed phase chromatography on a Biotage Agela C18 column (120 g, spherical 20-35 μm, 100 Å) eluting with ACN (from 5% to 95%) in aq. TFA (0.1%), yielding the title compound as a white solid (2.1 g).

Method A: Rt=1.39 min; [M+H]⁺=527, 529.

### Step 4: tert-butyl 4-((1-(2,6-dimethoxy-4-(4,4,5,5-tetramethyl-1,3,2-dioxaborolan-2-yl)phenethyl)piperidin-4-yl)oxy)piperidine-1-carboxylate

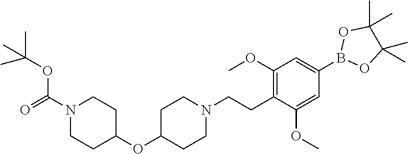 [SMILES: COc1cc(B2OC(C)(C)C(C)(C)O2)cc(OC)c1CCN1CCC(OC2CCN(C(=O)OC(C)(C)C)CC2)CC1]

To a 100 ml round bottom flask were added tert-butyl 4-((1-(4-bromo-2,6-dimethoxyphenethyl)piperidin-4-yl)oxy)piperidine-1-carboxylate (2.1 g, 4 mmol), BISPIN (1.32 g, 5.2 mmol), K₂CO₃ (1.38 g, 10 mmol), 1,4-dioxane (20 ml) and PdCl₂(dppf) (146 mg, 0.2 mmol). The RM was stirred at 100° C. for 16 h under N₂ atmosphere. Water (50 ml) was added, the mixture was extracted with EtOAc (2×75 ml), the combined organic phases were washed with brine (2×30 mL), dried over Na₂SO₄ and the residue was purified by reversed phase chromatography on a Biotage Agela C18 column (120 g, spherical 20-35 μm, 100 Å) eluting with ACN (from 5% to 95%) in aq. TFA (0.1%), yielding the title compound as a white solid (1.1 g).

Method A: Rt=1.16 min; [M+H]⁺=575.

### Intermediate 35

### 2-butyl-4-(3-methoxy-4-((1-(piperidin-4-ylmethyl)piperidin-4-yl)oxy)phenyl)-2,7-naphthyridin-1(2H)-one

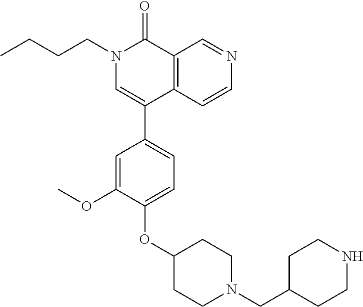 [SMILES: CCCCn1cc(-c2ccc(OC3CCN(CC4CCNCC4)CC3)c(OC)c2)c2ccncc2c1=O]

### Step 1: 4-(2-methoxy-4-(4,4,5,5-tetramethyl-1,3,2-dioxaborolan-2-yl)phenoxy)piperidine

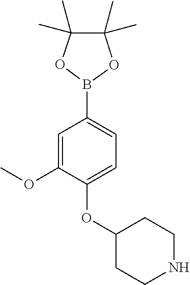 [SMILES: COc1cc(B2OC(C)(C)C(C)(C)O2)ccc1OC1CCNCC1]

To a 100 ml round bottom flask were added tert-butyl 4-(2-methoxy-4-(4,4,5,5-tetramethyl-1,3,2-dioxaborolan-2-yl)phenoxy)piperidine-1-carboxylate (intermediate 16, 5.48 g, 11.50 mmol), a solution of HCl (4 M) in 1,4-dioxane (15 ml) and MeOH (25 ml). The RM was stirred at RT for 2 h and concentrated. The residue was taken up in cold Et₂O and the mixture was filtered, yielding the corresponding hydrochloride salt of the title compound as a solid (4.33 g).

Method D: Rt=0.77 min; [M+H]⁺=334.

### Step 2: tert-butyl 4-((4-(2-methoxy-4-(4,4,5,5-tetramethyl-1,3,2-dioxaborolan-2-yl)phenoxy)piperidin-1-yl)methyl)piperidine-1-carboxylate

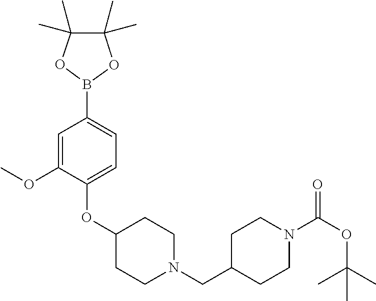 [SMILES: COc1cc(B2OC(C)(C)C(C)(C)O2)ccc1OC1CCN(CC2CCN(C(=O)OC(C)(C)C)CC2)CC1]

To a 100 ml round bottom flask were added 4-(2-methoxy-4-(4,4,5,5-tetramethyl-1,3,2-dioxaborolan-2-yl)phenoxy)piperidine hydrochloride salt (1.0 g, 2.46 mmol), tert-butyl 4-formylpiperidine-1-carboxylate (577 mg, 2.71 mmol), TEA (1 ml, 7.17 mmol), a solution of ZnCl₂ (0.5 M) in THE (5 ml, 2.5 mmol) and MeOH (15 ml). The RM was stirred at RT for 4 h. Solid NaBH₃CN (170 mg, 2.71 mmol) was added and the RM was stirred at RT for 20 h. The solvents were removed and the solid was directly used in the next step without further purification.

Method D: Rt=1.01 min; [M+H]⁺=531.

### Step 3: 4-(2-methoxy-4-(4,4,5,5-tetramethyl-1,3,2-dioxaborolan-2-yl)phenoxy)-1-(piperidin-4-ylmethyl)piperidine

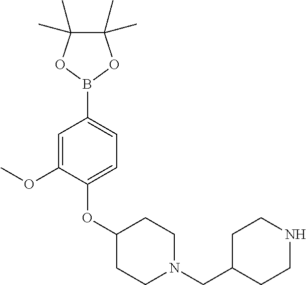 [SMILES: COc1cc(B2OC(C)(C)C(C)(C)O2)ccc1OC1CCN(CC2CCNCC2)CC1]

To a 100 ml round bottom flask were added tert-butyl 4-((4-(2-methoxy-4-(4,4,5,5-tetramethyl-1,3,2-dioxaborolan-2-yl)phenoxy)piperidin-1-yl)methyl)piperidine-1-carboxylate (2.46 mmol), a solution of HCl (4 M) in 1,4-dioxane (3 ml) and MeOH (15 ml). The RM was stirred at RT for 4 h, the solvents were removed and the residue was purified by reversed phase chromatography on a REDISEP® Gold HP C18 column (15.5 g) eluting with ACN (from 2% to 100%) in aq. TFA (0.1%), yielding the corresponding TFA salt of the title compound as a solid (695 mg).

Method D: Rt=0.65 min; [M+H]⁺=431.

### Step 4: 2-butyl-4-(3-methoxy-4-((1-(piperidin-4-ylmethyl)piperidin-4-yl)oxy)phenyl)-2,7-naphthyridin-1(2H)-one

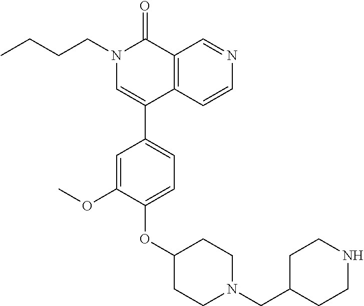 [SMILES: CCCCn1cc(-c2ccc(OC3CCN(CC4CCNCC4)CC3)c(OC)c2)c2ccncc2c1=O]

To a 25 ml round bottom flask were added under an argon atmosphere 4-(2-methoxy-4-(4,4,5,5-tetramethyl-1,3,2-dioxaborolan-2-yl)phenoxy)-1-(piperidin-4-ylmethyl)piperidine TFA salt (242 mg, 0.367 mmol), 4-bromo-2-butyl-2,7-naphthyridin-1(2H)-one (intermediate 32, 125 mg, 0.333 mmol), Na₂CO₃ (177 mg, 1.667 mmol), 1,4-dioxane (2 ml) and water (0.5 ml). Solid PdCl₂(dppf)—CH₂C2(25 mg, 0.034 mmol) was added and the RM was stirred at 100° C. for 2 h. The solvents were removed and the residue was purified by reversed phase chromatography on a REDISEP® Gold HP C18 column (15.5 g) eluting with ACN (from 2% to 100%) in aq. TFA (0.1%), yielding the corresponding TFA salt of the title compound as a solid (251 mg).

Method D: Rt=0.59 min; [M+H]⁺=505.

### Intermediate 36

### 2-butyl-4-(3,5-dimethoxy-4-((1-(piperidin-4-ylmethyl)piperidin-4-yl)methoxy)phenyl)-2,7-naphthyridin-1(2H)-one

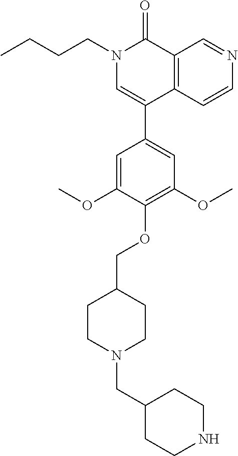 [SMILES: CCCCn1cc(-c2cc(OC)c(OCC3CCN(CC4CCNCC4)CC3)c(OC)c2)c2ccncc2c1=O]

### Step 1: tert-butyl 4-((4-(2-butyl-1-oxo-1,2-dihydro-2,7-naphthyridin-4-yl)-2,6-dimethoxyphenoxy)methyl)piperidine-1-carboxylate

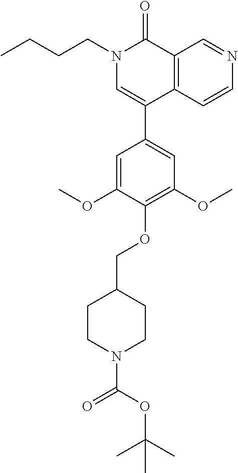 [SMILES: CCCCn1cc(-c2cc(OC)c(OCC3CCN(C(=O)OC(C)(C)C)CC3)c(OC)c2)c2ccncc2c1=O]

To a 25 ml round bottom flask were added under an argon atmosphere 4-bromo-2-butyl-2,7-naphthyridin-1(2H)-one (intermediate 32, 110 mg, 0.293 mmol), tert-butyl 4-((2,6-dimethoxy-4-(4,4,5,5-tetramethyl-1,3,2-dioxaborolan-2-yl)phenoxy)methyl)piperidine-1-carboxylate (intermediate 18,140 mg, 0.293 mmol), Na₂CO₃ (93 mg, 0.880 mmol), 1,4-dioxane (2 ml) and water (1 ml). Solid PdCl₂(dppf)-CH₂Cl₂ (22 mg, 0.030 mmol) was added and the RM was stirred at 100° C. for 2 h. The solvents were removed and the residue was purified by reversed phase chromatography on a REDISEP® Gold HP C18 column (15.5 g) eluting with ACN (from 2% to 100%) in aq. TFA (0.1%), yielding the title compound (123 mg) as a solid.

Method D: Rt=1.36 min; [M+H]⁺=552.

### Step 2: 2-butyl-4-(3,5-dimethoxy-4-(piperidin-4-ylmethoxy)phenyl)-2,7-naphthyridin-1(2H)-one

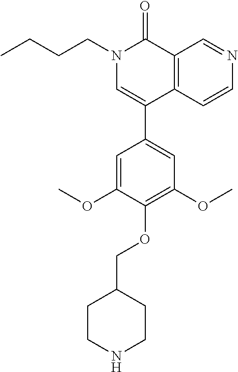 [SMILES: CCCCn1cc(-c2cc(OC)c(OCC3CCNCC3)c(OC)c2)c2ccncc2c1=O]

To a 25 ml round bottom flask were added tert-butyl 4-((4-(2-butyl-1-oxo-1,2-dihydro-2,7-naphthyridin-4-yl)-2,6-dimethoxyphenoxy)methyl)piperidine-1-carboxylate (118 mg, 0.214 mmol), TFA (0.5 ml, 6.49 mmol) and DCM (2 ml). The RM was stirred at RT for 1 h and the solvents were removed, yielding the corresponding TFA salt of the title compound as a solid (141 mg), which was used without further purification for the next step.

Method D: Rt=0.74 min; [M+H]⁺=452.

### Step 3: tert-butyl 4-((4-((4-(2-butyl-1-oxo-1,2-dihydro-2,7-naphthyridin-4-yl)-2,6-dimethoxyphenoxy)methyl)piperidin-1-yl)methyl)piperidine-1-carboxylate

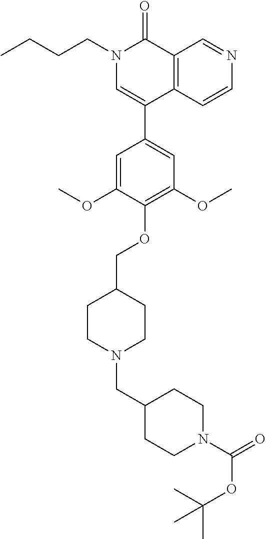 [SMILES: CCCCn1cc(-c2cc(OC)c(OCC3CCN(CC4CCN(C(=O)OC(C)(C)C)CC4)CC3)c(OC)c2)c2ccncc2c1=O]

To a 25 ml round bottom flask were added 2-butyl-4-(3,5-dimethoxy-4-(piperidin-4-ylmethoxy)phenyl)-2,7-naphthyridin-1(2H)-one TFA salt (121 mg, 0.214 mmol), tert-butyl 4-formylpiperidine-1-carboxylate (55 mg, 0.257 mmol), TEA (0.100 ml, 0.717 mmol), a solution of ZnCl₂ (0.5 M) in THF (0.450 ml, 0.225 mmol) and MeOH (2 ml). The RM was stirred at RT for 7 h and solid NaBH₃CN (14 mg, 0.223 mmol) was added. The RM was stirred at RT for 2 days, the solvents were removed and the residue was directly used for the next step without further purification.

Method D: Rt=0.96 min; [M+H]⁺=649.

### Step 4: 2-butyl-4-(3,5-dimethoxy-4-((1-(piperidin-4-ylmethyl)piperidin-4-yl)methoxy)phenyl)-2,7-naphthyridin-1(2H)-one

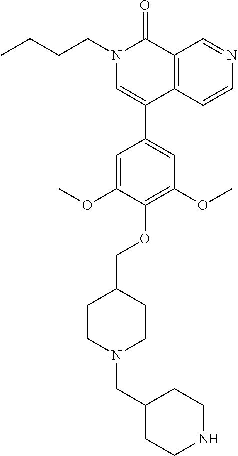 [SMILES: CCCCn1cc(-c2cc(OC)c(OCC3CCN(CC4CCNCC4)CC3)c(OC)c2)c2ccncc2c1=O]

To a 25 ml round bottom flask were added tert-butyl 4-((4-((4-(2-butyl-1-oxo-1,2-dihydro-2,7-naphthyridin-4-yl)-2,6-dimethoxyphenoxy)methyl)piperidin-1-yl)methyl)piperidine-1-carboxylate (0.214 mmol), TFA (0.5 ml, 6.49 mmol) and DCM (2 ml). The RM was stirred at RT for 1 h, the solvents were removed and the residue was purified by reversed phase chromatography on a REDISEP® Gold HP C18 column (15.5 g) eluting with ACN (from 2% to 100%) in aq. TFA (0.1%), yielding the corresponding TFA salt of the title compound as a solid (180 mg).

Method D: Rt=0.63 min; [M+H]⁺=549.

### Intermediate 37

### 2-butyl-4-(3,5-dimethoxy-4-((1-(piperidin-4-ylmethyl)piperidin-4-yl)oxy)phenyl)-2,7-naphthyridin-1(2H)-one

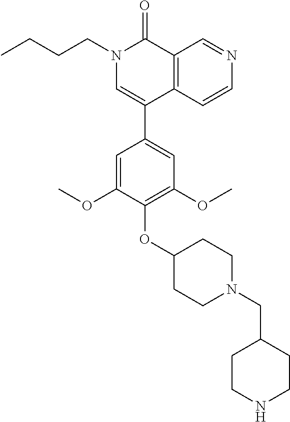 [SMILES: CCCCn1cc(-c2cc(OC)c(OC3CCN(CC4CCNCC4)CC3)c(OC)c2)c2ccncc2c1=O]

### Step 1: tert-butyl 4-(4-(2-butyl-1-oxo-1,2-dihydro-2,7-naphthyridin-4-yl)-2,6-dimethoxyphenoxy)piperidine-1-carboxylate

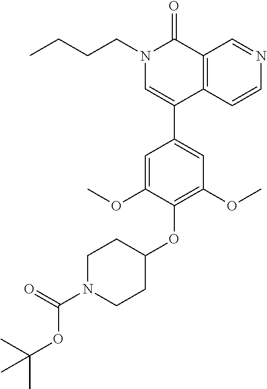 [SMILES: CCCCn1cc(-c2cc(OC)c(OC3CCN(C(=O)OC(C)(C)C)CC3)c(OC)c2)c2ccncc2c1=O]

To a 25 ml round bottom flask were added under an argon atmosphere 4-bromo-2-butyl-2,7-naphthyridin-1(2H)-one (intermediate 32, 150 mg, 0.400 mmol), tert-butyl 4-(2,6-dimethoxy-4-(4,4,5,5-tetramethyl-1,3,2-dioxaborolan-2-yl)phenoxy)piperidine-1-carboxylate (intermediate 17, 256 mg, 0.480 mmol), Na₂CO₃ (127 mg, 1.200 mmol), 1,4-dioxane (4 ml) and water (1 ml). Solid PdCl₂(dppf)-CH₂Cl₂ (30 mg, 0.041 mmol) was added and the RM was stirred at 100° C. for 2 h. The solvents were removed and the residue was purified by reversed phase chromatography on a REDISEP® Gold HP C18 column (15.5 g) eluting with ACN (from 2% to 100%) in aq. TFA (0.1%), yielding the title compound as a solid (215 mg).

Method D: Rt=1.31 min; [M+H]⁺=538.

### Step 2: 2-butyl-4-(3,5-dimethoxy-4-(piperidin-4-yloxy)phenyl)-2,7-naphthyridin-1(2H)-one

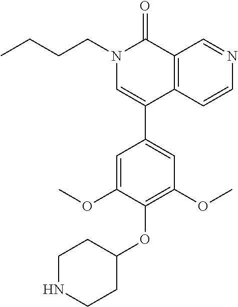 [SMILES: CCCCn1cc(-c2cc(OC)c(OC3CCNCC3)c(OC)c2)c2ccncc2c1=O]

To a 25 ml round bottom flask were added tert-butyl 4-(4-(2-butyl-1-oxo-1,2-dihydro-2,7-naphthyridin-4-yl)-2,6-dimethoxyphenoxy)piperidine-1-carboxylate (215 mg, 0.380 mmol), TFA (1 ml, 12.98 mmol) and DCM (2 ml). The RM was stirred at RT for 1 h and evaporated to dryness, yielding the corresponding TFA salt of the title compound as a solid (216 mg).

Method D: Rt=0.72 min; [M+H]⁺=438.

### Step 3: tert-butyl 4-((4-(4-(2-butyl-1-oxo-1,2-dihydro-2,7-naphthyridin-4-yl)-2,6-dimethoxyphenoxy)piperidin-1-yl)methyl)piperidine-1-carboxylate

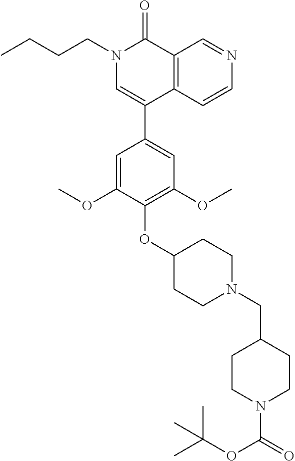 [SMILES: CCCCn1cc(-c2cc(OC)c(OC3CCN(CC4CCN(C(=O)OC(C)(C)C)CC4)CC3)c(OC)c2)c2ccncc2c1=O]

To a 25 ml round bottom flask were added 2-butyl-4-(3,5-dimethoxy-4-(piperidin-4-yloxy)phenyl)-2,7-naphthyridin-1(2H)-one TFA salt (210 mg, 0.380 mmol), tert-butyl 4-formylpiperidine-1-carboxylate (100 mg, 0.469 mmol), TEA (0.150 ml, 1.076 mmol), a solution of ZnCl₂ (0.5 M) in THE (0.800 ml, 0.400 mmol) and MeOH (4 ml). The RM was stirred at RT for 7 h, solid NaBH₃CN (25 mg, 0.398 mmol) was added and the RM was stirred at RT for 20 h. The solvents were removed and the residue, containing the title compound, was directly used for the next step without further purification.

Method D: Rt=0.96 min; [M+H]⁺=635.

### Step 4: 2-butyl-4-(3,5-dimethoxy-4-((1-(piperidin-4-ylmethyl)piperidin-4-yl)oxy)phenyl)-2,7-naphthyridin-1(2H)-one

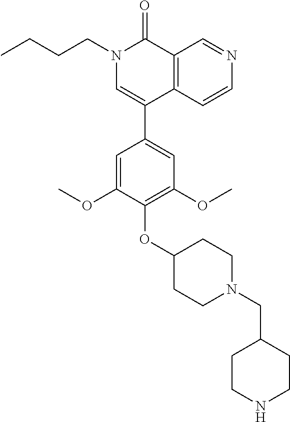 [SMILES: CCCCn1cc(-c2cc(OC)c(OC3CCN(CC4CCNCC4)CC3)c(OC)c2)c2ccncc2c1=O]

To a 25 ml round bottom flask were added tert-butyl 4-((4-(4-(2-butyl-1-oxo-1,2-dihydro-2,7-naphthyridin-4-yl)-2,6-dimethoxyphenoxy)piperidin-1-yl)methyl)piperidine-1-carboxylate (0.380 mmol), TFA (1 ml, 12.98 mmol) and DCM (2 ml). The RM was stirred at RT for 1 h, the solvents were removed and the residue was purified by reversed phase chromatography on a REDISEP® Gold HP C18 column (15.5 g) eluting with ACN (from 2% to 100%) in aq. TFA (0.1%), yielding the corresponding TFA salt of the title compound as a solid (259 mg).

Method D: Rt=0.61 min; [M+H]⁺=535.

### Intermediate 38

### Perfluorophenyl 4-chloro-3-(2,4-dioxotetrahydropyrimidin-1(2H)-yl)benzoate

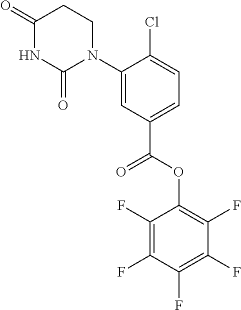 [SMILES: O=C1CCN(c2cc(C(=O)Oc3c(F)c(F)c(F)c(F)c3F)ccc2Cl)C(=O)N1]

To a 50 ml round bottom flask were added 4-chloro-3-(2,4-dioxotetrahydropyrimidin-1(2H)-yl)benzoic acid (intermediate 24, 500 mg, 1.89 mmol), perfluorophenyl 2,2,2-trifluoroacetate (1.32 g, 4.73 mmol) and DMF (5 ml). The RM was stirred at 0° C. for 10 min, DIEA (975 mg, 7.56 mmol) was added dropwise over 5 min and the RM was stirred at RT for 2 h. The mixture was added into EtOAc (100 ml), the organic phase was washed with water (2×50 ml) and brine (50 ml) and the residue was purified by chromatography on silica gel eluting with EtOAc (from 0% to 20%) in PE, yielding the title compound as a solid (750 mg).

Method H: Rt=2.04 min; [M+H]⁺=435.

### Intermediate 39: 2-(3-((2,4-dioxotetrahydropyrimidin-1(2H)-yl)methyl)-2-oxopyridin-1(2H)-yl)acetaldehyde

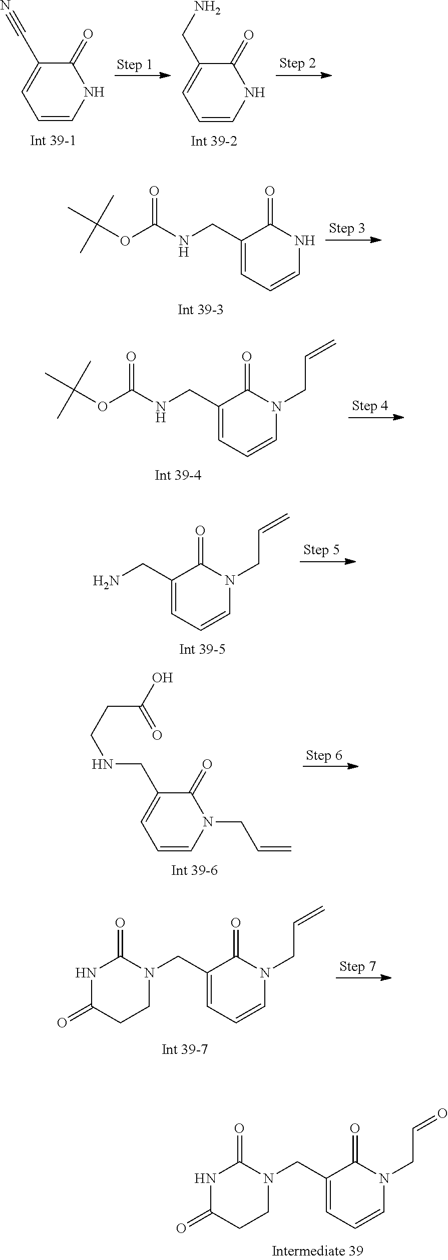

### Step 1: 3-(aminomethyl)pyridin-2(1H)-one (Int 39-2)

To a 1 L round bottom flask was added 2-oxo-1,2-dihydropyridine-3-carbonitrile (Int 39-1, 12 g, 100 mmol), Raney Ni (3 g), a solution of NH₃ (7 M) in MeOH (100 mL) and MeOH (150 mL). The RM was stirred under an atmosphere of H₂ (1 atm) at RT for 48 h, filtered, and the filtrate was concentrated to provide Int 39-2 as a yellow oil (13.5 g), which was used in the next step without further purification. LC-MS Method C: Rt=0.48 min; [M+H]⁺=125.

### Step 2: tert-butyl ((2-oxo-1,2-dihydropyridin-3-yl)methyl)carbamate (Int 39-3)

To a 1 L round bottom flask was added 3-(aminomethyl)pyridin-2(1H)-one (Int 39-2, 13.5 g, 100 mmol), DIEA (25.8 g, 200 mmol), MeOH (200 mL), DCM (300 mL), and di-tert-butyl dicarbonate (21.8 g, 100 mmol). The RM was stirred at RT for 16 h, concentrated and the residue was purified by chromatography on silica gel eluting with 0% to 8% MeOH in DCM to afford the title compound Int 39-3 as an oil (10.0 g). LC-MS Method B: Rt=1.61 min; [M+H]⁺=225.

### Step 3: tert-butyl ((1-allyl-2-oxo-1,2-dihydropyridin-3-yl)methyl)carbamate (Int 39-4)

To a 250 mL round bottom flask was added tert-butyl ((2-oxo-1,2-dihydropyridin-3-yl)methyl)carbamate (Int 39-3, 10.0 g, 45 mmol), K₂CO₃ (12.4 g, 90 mmol), DMF (80 mL), and 3-bromoprop-1-ene (8.1 g, 67 mmol). The RM was stirred at RT for 16 h, filtered, and the filtrate was added into water (500 mL). The mixture was extracted with EtOAc (4×300 mL) and the combined organic phases were dried over Na₂SO₄, filtered and concentrated to provide the title compound Int 39-4 as an oil (14.0 g). LC-MS Method B: Rt=1.78 min; [M+H]⁺=265.

### Step 4: 1-allyl-3-(aminomethyl)pyridin-2(1H)-one (Int 39-5)

To a 1 L round bottom flask was added tert-butyl ((1-allyl-2-oxo-1,2-dihydropyridin-3-yl)methyl)carbamate (Int 39-4, 14.0 g), DCM (300 mL) and a solution of HCl (4 M) in 1,4-dioxane (50 mL). The RM was stirred at RT for 16 h, the solvents were removed and the resulting residue was purified by reversed phase chromatography on a Biotage Agela C18 column (120 g, spherical 20-35 μm, 100 Å) eluting with 5% to 40% ACN in aq. ammonium hydrogen carbonate (0.1%) to provide the title compound Int 39-5 as an oil (7.2 g). LC-MS

Method B: Rt=1.14 min; [M+H]⁺=165.

### Step 5: 3-(((1-allyl-2-oxo-1,2-dihydropyridin-3-yl)methyl)amino)propanoic Acid (Int 39-6)

To a 250 mL round bottom flask was added 1-allyl-3-(aminomethyl)pyridin-2(1H)-one (Int 39-5, 3.28 g, 20 mmol), acrylic acid (4.32 g, 60 mmol), and toluene (100 mL). The RM was stirred at 100° C. for 18 h, concentrated, and the crude material Int 39-6 was used in the next step without further purification. LC-MS Method O: Rt=0.34 min; [M+H]⁺=237.

### Step 6: 1-((1-allyl-2-oxo-1,2-dihydropyridin-3-yl)methyl)dihydropyrimidine-2,4(1H,3H)-dione (Int 39-7)

To a 250 mL round bottom flask was added 3-(((1-allyl-2-oxo-1,2-dihydropyridin-3-yl)methyl)amino)propanoic acid (crude Int 39-6, 8 g), urea (3.6 g, 60 mmol), and acetic acid (40 mL). The RM was stirred at 120° C. for 18 h, concentrated, and the crude residue was purified by reversed phase chromatography on a Biotage Agela C18 column (120 g, spherical 20-35 μm, 100 Å) eluting with 5% to 50% ACN in aq. ammonium hydrogen carbonate (0.1%) to provide the title compound Int 39-7 as a solid (3.4 g). LC-MS Method B: Rt=1.40 min; [M+H]⁺=262.

### Step 7: 2-(3-((2,4-dioxotetrahydropyrimidin-1(2H)-yl)methyl)-2-oxopyridin-1(2H)-yl)acetaldehyde (Intermediate 39)

To a 250 mL round bottom flask was added 1-((1-allyl-2-oxo-1,2-dihydropyridin-3-yl)methyl)dihydropyrimidine-2,4(1H,3H)-dione (Int 39-7, 3.9 g, 15 mmol), THE (120 mL), and a solution of OsO₄ (4%) in water (8 mL). The RM was stirred under an atmosphere of nitrogen at RT for 45 min. Solid NaIO₄ (9.6 g, 45 mmol) was then added and the RM was stirred under an atmosphere of nitrogen at RT for 16 h. The mixture was filtered, the solvents were removed, and the resulting residue was purified by reversed phase chromatography on a Biotage Agela C18 column (120 g, spherical 20-35 μm, 100 Å) eluting with 0% to 30% ACN in aq. ammonium hydrogen carbonate (0.1%) to provide the title compound Intermediate 39 as a solid (3.6 g).

LC-MS Method P: Rt=0.42 min; [M+H]⁺=264.

### Intermediate 40: 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione

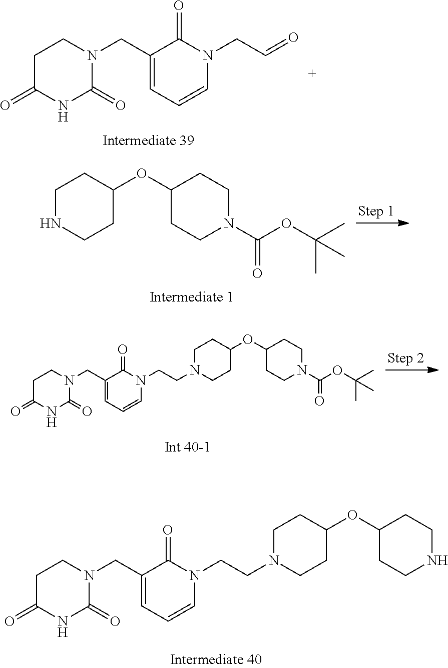 [SMILES: O=CCn1cccc(CN2CCC(=O)NC2=O)c1=O ; CC(C)(C)OC(=O)N1CCC(OC2CCNCC2)CC1 ; CC(C)(C)OC(=O)N1CCC(OC2CCN(CCn3cccc(CN4CCC(=O)NC4=O)c3=O)CC2)CC1 ; O=C1CCN(Cc2cccn(CCN3CCC(OC4CCNCC4)CC3)c2=O)C(=O)N1]

### Step 1: tert-butyl 4-(1-(2-(3-((2,4-dioxotetrahydropyrimidin-1(2H)-yl)methyl)-2-oxopyridin-1(2H)-yl)ethyl)piperidin-4-yloxy)piperidine-1-carboxylate (Int 40-1)

To a 250 mL round bottom flask was added 2-(3-((2,4-dioxotetrahydropyrimidin-1(2H)-yl)methyl)-2-oxopyridin-1(2H)-yl)acetaldehyde (Intermediate 39, 3.6 g, 13.6 mmol), tert-butyl 4-(piperidin-4-yloxy)piperidine-1-carboxylate (Intermediate 1, 3.86 g, 13.6 mmol), a solution of ZnCl₂ (1 M) in THE (20.4 mL, 20.4 mmol), and DMSO (40 mL). The RM was stirred at RT for 2 h and solid NaBH₃CN (2.57 g, 40.8 mmol) and MeOH (8 mL) were then added. The RM was stirred at RT for 16 h, concentrated, and purified by reversed phase chromatography on a Biotage Agela C18 column (120 g, spherical 20-35 μm, 100 Å) eluting with 5% to 60% ACN in aq. NH₄HCO₃ (0.1%) to provide the title compound Int 40-1 as a solid (2.8 g). LC-MS Method C: Rt=1.81 min; [M+H]⁺=532.

### Step 2: 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione (Intermediate 40)

To a 250 mL round bottom flask was added tert-butyl 4-(1-(2-(3-((2,4-dioxotetrahydropyrimidin-1(2H)-yl)methyl)-2-oxopyridin-1(2H)-yl)ethyl)piperidin-4-yloxy)piperidine-1-carboxylate (Int 40-1, 2.8 g, 5.2 mmol), DCM (30 mL), and a solution of HCl (4 M) in 1,4-dioxane (10 mL) and the resulting mixture was stirred at RT for 6 h. The RM was then concentrated, and the crude residue purified by reversed phase chromatography on a Biotage Agela C18 column (120 g, spherical 20-35 μm, 100 Å) eluting with 0% to 50% ACN in aq. NH₄HCO₃ (0.1%) to provide the title compound Intermediate 40 as a solid (1.8 g). LC-MS

Method B: Rt=1.36 min; [M+H]⁺=432.

### Intermediate 41: 2,6-dimethoxy-4-(2-butyl-1-oxo-1,2-dihydro-2,7-naphthyridin-4-yl)benzaldehyde

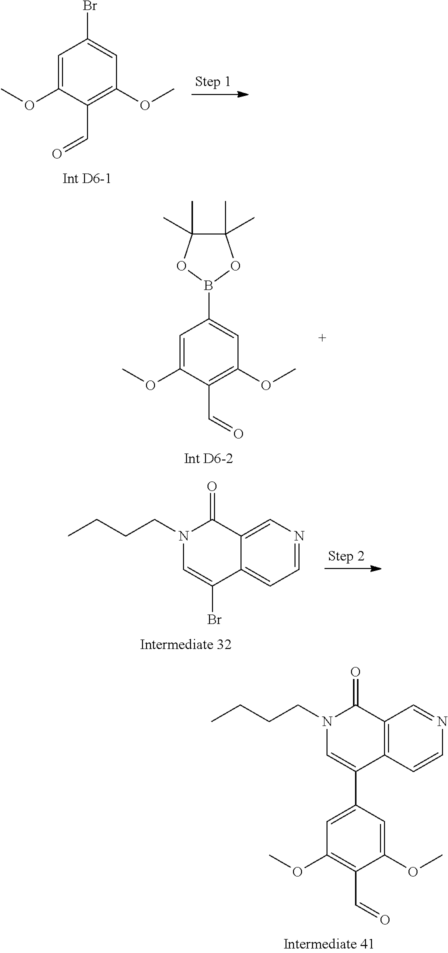

### Step 1: 2,6-dimethoxy-4-(4,4,5,5-tetramethyl-1,3,2-dioxaborolan-2-yl)benzaldehyde (Int D6-2)

To a 500 ml round bottom flask were added under an argon atmosphere 4-bromo-2,6-dimethoxybenzaldehyde (Int D6-1, 8 g, 31.7 mmol), BISPIN (10 g, 39.4 mmol), dppf (527 mg, 0.950 mmol), KOAc (9323 mg, 95 mmol) and 1,4-dioxane (100 ml). Solid PdCl₂(dppf) (695 mg, 0.950 mmol) was added and the RM was stirred at 90° C. overnight. The mixture was cooled to RT, filtered over CELITE®, the solids were washed with EtOAc and the combined filtrates were washed with an aq. solution of HCl (0.1N) and brine, dried over MgSO₄ and the residue was purified by chromatography on silica gel eluting with EtOAc (from 5% to 100%) in CHX, yielding the title compound Int D6-2 as a solid (7.42 g).

LC-MS Method D: Rt=1.12 min; [M+H]⁺=293.

### Step 2: 2,6-dimethoxy-4-(2-butyl-1-oxo-1,2-dihydro-2,7-naphthyridin-4-yl)benzaldehyde (Intermediate 41)

To a 100 ml round bottom flask were added under an argon atmosphere 4-bromo-2-butyl-2,7-naphthyridin-1(2H)-one (Intermediate 32, 2.19 g, 6.54 mmol), 2,6-dimethoxy-4-(4,4,5,5-tetramethyl-1,3,2-dioxaborolan-2-yl)benzaldehyde (Int D6-2, 2.17 g, 6.54 mmol), Na₂CO₃ (2.08 g, 19.63 mmol), 1,4-dioxane (40 ml) and water (10 ml). Solid PdCl₂(dppf)-CH₂Cl₂(484 mg, 0.654 mmol) was added and the RM was stirred at 100° C. for 2 h. The mixture was concentrated and the residue was purified by chromatography on silica gel eluting with EtOAc (from 0% to 95%) in DCM, yielding the title compound Intermediate 41 as a solid (1.45 g).

LC-MS Method D: Rt=0.95 min; [M+H]⁺=367.

### Intermediate 42: tert-butyl (3R,4S)-3-fluoro-4-(tosyloxy)piperidine-1-carboxylate

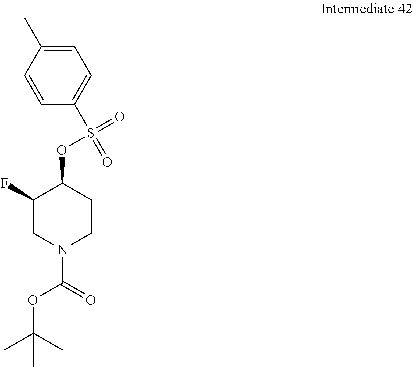 [SMILES: Cc1ccc(S(=O)(=O)O[C@H]2CCN(C(=O)OC(C)(C)C)C[C@H]2F)cc1]

Intermediate 42 was prepared according to the procedure described in WO 2015/022662 A1 on pages 126 to 127, described as isomer 4.3-2 and eluting under the described chiral HPLC separation conditions as peak-2 at 11.406 min.

### Intermediate 43: tert-butyl (3R,4R)-3-fluoro-4-(piperidin-4-yloxy)piperidine-1-carboxylate

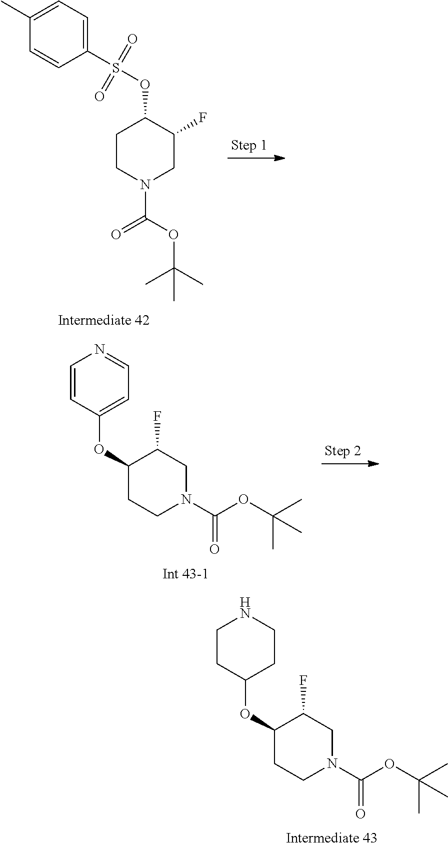 [SMILES: Cc1ccc(S(=O)(=O)O[C@H]2CCN(C(=O)OC(C)(C)C)C[C@H]2F)cc1 ; CC(C)(C)OC(=O)N1CC[C@@H](Oc2ccncc2)[C@H](F)C1 ; CC(C)(C)OC(=O)N1CC[C@@H](OC2CCNCC2)[C@H](F)C1]

### Step 1: tert-butyl (3R,4R)-3-fluoro-4-(pyridin-4-yloxy)piperidine-1-carboxylate (Int 43-1)

To a solution of pyridin-4-ol (7.64 g, 80 mmol) in DMF (250 ml) under a nitrogen atmosphere was added solid Cs₂CO₃ (39.3 g, 121 mmol), tert-butyl (3R,4S)-3-fluoro-4-(tosyloxy)piperidine-1-carboxylate (Intermediate 42, 30 g, 80 mmol) and DMF (6 ml). The RM was stirred at 100° C. for 7 h and allowed to cool to RT. The mixture was concentrated and the residue was dissolved in a mixture of EtOAc and water. The aq. phase was extracted with EtOAc, the combined organic phases were washed with a mixture of a sat. aq. solution of NaHCO₃ and water, an aq. solution of LiBr (0.1 M) and brine and dried over MgSO₄. The residue was purified by chromatography on silica gel eluting with MeOH (from 0% to 20%) in DCM, yielding the title compound tert-butyl (3R,4R)-3-fluoro-4-(pyridin-4-yloxy)piperidine-1-carboxylate, Int 43-1, as an oil (13.3 g).

Method LCMS_MLG8: Rt=0.47 min; [M+H]⁺=296.

### Step 2: tert-butyl (3R,4R)-3-fluoro-4-(piperidin-4-yloxy)piperidine-1-carboxylate (Intermediate 43)

To a solution of tert-butyl (3R,4R)-3-fluoro-4-(pyridin-4-yloxy)piperidine-1-carboxylate (13.25 g, 42.50 mmol) in EtOH (250 ml) was added 4-methylbenzenesulfonic acid hydrate (8.5 g, 44.70 mmol) and PtO₂ (1.5 g). The RM was stirred under an atmosphere of hydrogen for 24 h. PtO₂ (1 g) was added and the RM was stirred under an atmosphere of hydrogen overnight. The mixture was filtered on CELITE® and the solids were washed with EtOH. The combined filtrates were concentrated and the residue was purified by chromatography on silica gel eluting with a mixture of DCM, MeOH, an aq. solution of NH₄OH (25%) (80:20:1) (from 0% to 80%) in DCM, yielding the title compound tert-butyl (3R,4R)-3-fluoro-4-(piperidin-4-yloxy)piperidine-1-carboxylate, Intermediate 43, as a solid 4-methylbenzenesulfonate salt (12.7 g).

Method LCMS_MLG2: Rt=0.48 min; [M+H]⁺=303.

¹H NMR (400 MHz, DMSO-d₆) δ[ppm] 8.29 (s, 2H), 7.55-7.38 (m, 2H), 7.23-7.00 (m, 2H), 4.50-4.26 (m, 1H), 3.83-3.61 (m, 3H), 3.52-3.40 (m, 1H), 3.31-3.26 (m, 1H), 3.22-3.17 (m, 2H), 3.15-3.11 (m, 1H), 3.03-2.90 (m, 2H), 2.29 (s, 3H), 1.98-1.87 (m, 2H), 1.86-1.77 (m, 1H), 1.73-1.58 (m, 2H), 1.50-1.41 (m, 1H), 1.39 (s, 9H).

### Intermediate 45: 4-bromo-2,6-difluorobenzaldehyde

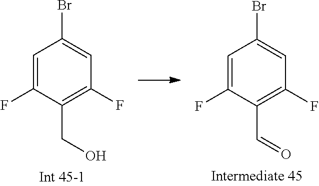 [SMILES: OCc1c(F)cc(Br)cc1F ; O=Cc1c(F)cc(Br)cc1F]

To a solution of (4-bromo-2,6-difluorophenyl)methanol (Int 45-1, 2 g, 8.8 mmol) in acetone (100 ml) was added manganese dioxide (15 g, 173.0 mmol) and the RM was stirred vigorously at RT for 20 h. The mixture was filtered, the filtrate was concentrated and the residue was purified by chromatography on silica gel eluting with EtOAc (20%) in CHX, yielding the title compound 4-bromo-2,6-difluorobenzaldehyde, Intermediate 45, as a solid (1.77 g).

Method LCMS_MLG2: Rt=0.86 min; [M+H]⁺=(no ionization)

¹H NMR (600 MHz, DMSO-d₆) δ[ppm] 10.15 (s, 1H), 7.80-7.55 (m, 2H).

### Intermediate 46: 2,6-difluoro-4-(4,4,5,5-tetramethyl-1,3,2-dioxaborolan-2-yl)benzaldehyde

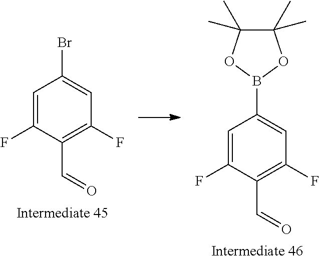 [SMILES: O=Cc1c(F)cc(Br)cc1F ; CC1(C)OB(c2cc(F)c(C=O)c(F)c2)OC1(C)C]

To a mixture of 4-bromo-2,6-difluorobenzaldehyde (Intermediate 45, 5.0 g, 22.62 mmol), BISPIN (6.89 g, 27.10 mmol), dppf (376 mg, 0.68 mmol) and KOAc (6.66 g, 67.90 mmol) in 1,4-dioxane (50 ml) under an argon atmosphere was added PdCl₂(dppf) (497 mg, 0.68 mmol) and the RM was heated at 90° C. for 24 h. The mixture was cooled to RT, filtered over CELITE®, the solids were washed with EtOAc, the filtrate was concentrated, adsorbed on ISOLUTE® and purified by chromatography on silica gel eluting with EtOAc (from 0% to 100%) in CHX, yielding the title compound 2,6-difluoro-4-(4,4,5,5-tetramethyl-1,3,2-dioxaborolan-2-yl)benzaldehyde, Intermediate 46, as a solid (5.62 g).

¹H NMR (400 MHz, DMSO-d₆) δ[ppm] 10.24 (s, 1H), 7.42-7.28 (m, 2H), 1.31 (s, 12H).

### Intermediate 47: (3-fluoro-4-formyl-5-methoxyphenyl)boronic Acid

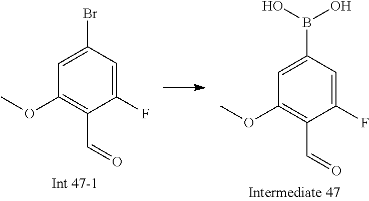

To a mixture of 4-bromo-2-fluoro-6-methoxybenzaldehyde (Int 47-1, 707 mg, 2.97 mmol), BISPIN (906 mg, 3.57 mmol), dppf (50 mg, 0.090 mmol) and KOAc (875 mg, 8.92 mmol) in 1,4-dioxane (15 ml) under an argon atmosphere was added PdCl₂(dppf) (66 mg, 0.090 mmol) and the RM was heated at 90° C. for 2 days. The RM was cooled to RT, PdCl₂(dppf) (66 mg, 0.090 mmol) was added under an argon atmosphere and the RM was heated at 100° C. for 4 h. The mixture was cooled to RT and filtered over CELITE®, the solids were washed with EtOAc, the filtrate was washed with an aq solution of HCl (0.1 M) and brine, dried over MgSO₄ and concentrated. The residue was purified by chromatography on silica gel eluting with EtOAc (from 0% to 80%) in CHX, yielding the title compound (3-fluoro-4-formyl-5-methoxyphenyl)boronic acid, Intermediate 47, as a solid (612 mg).

Method LCMS_MLG1: Rt=0.64 min; [M+H]⁺=199.

### Intermediate 48: 4-bromo-2-hexyl-2,7-naphthyridin-1(2H)-one

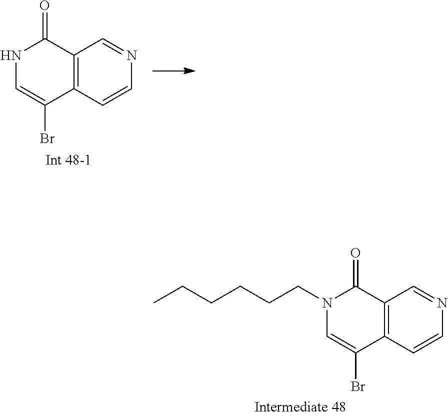 [SMILES: O=c1[nH]cc(Br)c2ccncc12 ; CCCCCCn1cc(Br)c2ccncc2c1=O]

### Step 1: 4-bromo-2-hexyl-2,7-naphthyridin-1(2H)-one (Intermediate 48)

To a mixture of 4-bromo-2,7-naphthyridin-1(2H)-one (Int 48-1, 500 mg, 2.177 mmol) and solid K₂CO₃ (903 mg, 6.53 mmol) in THF (8 ml) under an argon atmosphere was dropwise added 1-iodohexane (0.380 ml, 2.451 mmol) at RT and the RM was stirred at 70° C. for 20 h. 1-Iodohexane (0.120 ml, 0.797 mmol) was added and stirring was continued at 70° C. for 3 h. The mixture was added into water and the aq. phase was extracted with EtOAc. The organic phase was washed with water and brine, dried over MgSO₄ and concentrated. The residue was purified by chromatography on silica gel eluting with EtOAc (from 0% to 100%) in CHX, yielding the title compound 4-bromo-2-hexyl-2,7-naphthyridin-1(2H)-one, Intermediate 48, as a solid (438 mg).

Method LCMS_MLG1: [M+H]⁺=309 and 311.

### Intermediate 49: 3,4-dimethyl-1-propyl-5-(4,4,5,5-tetramethyl-1,3,2-dioxaborolan-2-yl)pyridin-2(1H)-one

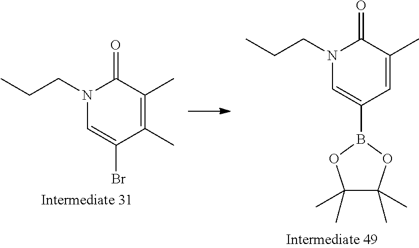 [SMILES: CCCn1cc(Br)c(C)c(C)c1=O ; CCCn1cc(B2OC(C)(C)C(C)(C)O2)cc(C)c1=O]

To a mixture of 5-bromo-3,4-dimethyl-1-propylpyridin-2(1H)-one (Intermediate 31, 1.57 g, 6.43 mmol), BISPIN (2.286 g, 9.00 mmol) and KOAc (1.262 g, 12.86 mmol) in 1,4-dioxane (32.2 ml) under an atmosphere of nitrogen was added PdCl₂(dppf)-CH₂Cl₂ (0.263 g, 0.322 mmol) and the RM was heated at 90° C. for 2 h. The mixture was cooled to RT and concentrated, the residue was taken up in DCM and the organic phase was washed with water and brine, dried over Na₂SO₄, filtered over CELITE® and the filtrate was concentrated. The residue was purified by column chromatography on silica gel, eluting with EtOAc (from 0% to 40%) in heptane, yielding the title compound 3,4-dimethyl-1-propyl-5-(4,4,5,5-tetramethyl-1,3,2-dioxaborolan-2-yl)pyridin-2(1H)-one, Intermediate 49, as a solid.

Method LCMS-ACQ-QDA #KAB0746—basic: Rt=1.11 min; [M+H]⁺=292.

### Intermediate 50: 2-methyl-4-(4,4,5,5-tetramethyl-1,3,2-dioxaborolan-2-yl)-2,7-naphthyridin-1(2H)-one

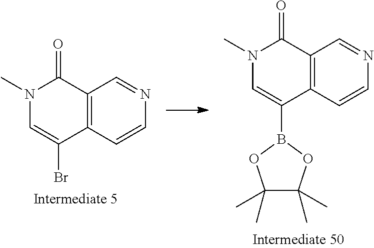 [SMILES: Cn1cc(Br)c2ccncc2c1=O ; Cn1cc(B2OC(C)(C)C(C)(C)O2)c2ccncc2c1=O]

To a mixture of 4-bromo-2-methyl-2,7-naphthyridin-1(2H)-one (Intermediate 5, 7.0 g, 29.3 mmol), BISPIN (14.9 g, 58.6 mmol) and KOAc (8.6 g, 87.8 mmol) in 1,4-dioxane (100 ml) under a nitrogen atmosphere was added Pd(dppf)Cl₂ (2.1 g, 2.93 mmol) and the RM was stirred at 100° C. for 16 h. The mixture was diluted with EtOAc (500 ml) and water (100 ml), the aq. phase was extracted with EtOAc (4×500 ml), the combined organic phases were washed with brine, dried over Na₂SO₄ and concentrated. The residue was purified by chromatography on silica gel eluting with EtOAc (from 0% to 50%) in DCM. Fractions containing the title compound were combined and concentrated. The residue was triturated with MTBE (20 ml), the mixture was filtered and the solids were dried, yielding the title compound 2-methyl-4-(4,4,5,5-tetramethyl-1,3,2-dioxaborolan-2-yl)-2,7-naphthyridin-1(2H)-one, Intermediate 50, as a solid (1.7 g).

¹H NMR (400 MHz, DMSO-d₆) δ[ppm] 9.36 (s, 1H), 8.74 (d, J=5.6 Hz, 1H), 8.11 (d, J=5.6 Hz, 1H), 8.04 (s, 1H), 3.58 (s, 3H), 1.34 (s, 12H).

### Intermediate 51: 2-butyl-4-(4,4,5,5-tetramethyl-1,3,2-dioxaborolan-2-yl)-2,7-naphthyridin-1(2H)-one

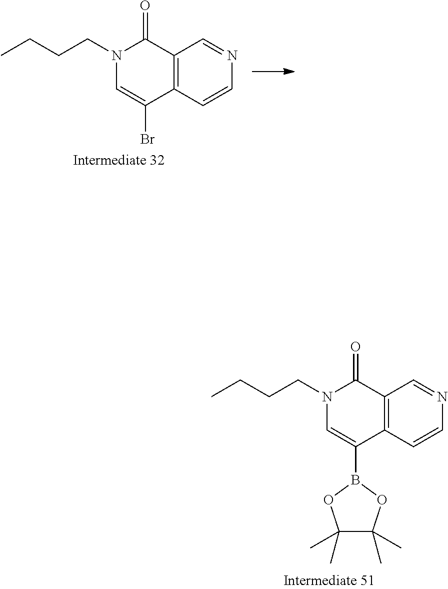 [SMILES: CCCCn1cc(Br)c2ccncc2c1=O ; CCCCn1cc(B2OC(C)(C)C(C)(C)O2)c2ccncc2c1=O]

To a mixture of 4-bromo-2-butyl-2,7-naphthyridin-1(2H)-one (Intermediate 32, 30 g, 0.1 mol), BISPIN (49.2 g, 0.15 mol) and KOAc (24.54 g, 0.25 mol) in 1,4-dioxane (630 ml) under a nitrogen atmosphere was added Pd(dppf)Cl₂ (7.32 g, 0.01 mol) and the RM was stirred at 100° C. for 16 h. The mixture was cooled to RT and water (100 ml) was added. The aq. phase was extracted with EtOAc (3×50 ml) and the combined organic phases were washed with brine (2×50 ml), dried over Na₂SO₄ and concentrated. The residue was purified by chromatography on silica gel eluting with EtOAc (from 0% to 20%) in PE, yielding the title compound 2-butyl-4-(4,4,5,5-tetramethyl-1,3,2-dioxaborolan-2-yl)-2,7-naphthyridin-1(2H)-one, Intermediate 51, as a solid (18.6 g).

¹H NMR (400 MHz, chloroform-d₃) δ [ppm] 9.60 (s, 1H), 8.71 (d, J=5.7 Hz, 1H), 8.20 (dd, J=5.7, 0.6 Hz, 1H), 7.81 (s, 1H), 4.15-3.95 (m, 2H), 1.92-1.65 (m, 2H), 1.58-1.32 (m, 14H), 0.98 (t, J=7.4 Hz, 3H).

### Intermediate 52: 2-(4-(4,5-dimethyl-6-oxo-1-propyl-1,6-dihydropyridin-3-yl)-2,6-dimethoxyphenyl)acetaldehyde

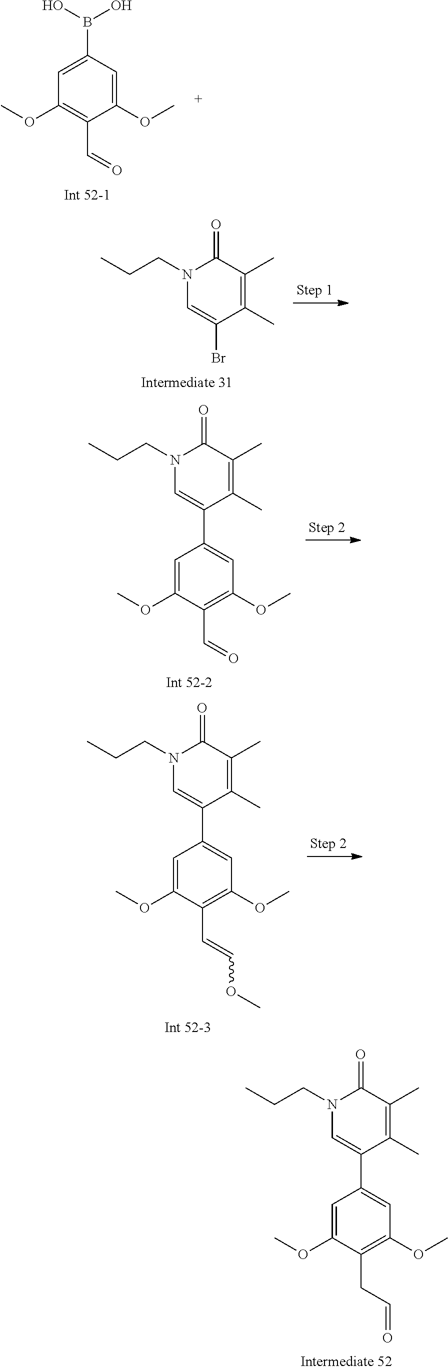

### Step 1: 4-(4,5-dimethyl-6-oxo-1-propyl-1,6-dihydropyridin-3-yl)-2,6-dimethoxybenzaldehyde (Int 52-2)

To a mixture of (4-formyl-3,5-dimethoxyphenyl)boronic acid (Int 52-1, 1.240 g, 5.73 mmol), 5-bromo-3,4-dimethyl-1-propylpyridin-2(1H)-one (Intermediate 31, 1.076 g, 4.41 mmol) and Na₂CO₃ (1.401 g, 13.22 mmol) in a mixture of 1,4-dioxane (30 ml) and water (47.5 ml) under an argon atmosphere was added PdCl₂(dppf)-CH₂Cl₂(169 mg, 0.229 mmol) and the RM was heated at 100° C. for 2.5 h. The mixture was cooled to RT and concentrated, the residue was dissolved in DCM and the mixture was filtered through CELITE®, the filtrate was concentrated and the residue purified by chromatography on silica gel eluting with EtOAc (from 0% to 100%) in heptane, yielding the title compound 4-(4,5-dimethyl-6-oxo-1-propyl-1,6-dihydropyridin-3-yl)-2,6-dimethoxybenzaldehyde, Int 52-2, as a solid (1.3 g).

Method LCMS_MLG7: Rt=0.79 min; [M+H]⁺=330.

### Step 2: (E,Z)-5-(3,5-dimethoxy-4-(2-methoxyvinyl)phenyl)-3,4-dimethyl-1-propylpyridin-2(1H)-one (Int 52-3)

To a solution of (methoxymethyl)triphenylphosphonium chloride (4.06 g, 11.84 mmol) in THE (10 ml) at 0° C. was added a solution of t-BuOK (1 M) in THE (15.47 ml, 14.47 mmol) and the RM was stirred at 0° C. for 30 min. A solution of 4-(4,5-dimethyl-6-oxo-1-propyl-1,6-dihydropyridin-3-yl)-2,6-dimethoxybenzaldehyde (Int 52-2, 1.3 g, 3.95 mmol) in THE (20 ml) was dropwise added and the RM was stirred at RT for 1 h, then at 70° C. for 80 min. The mixture was concentrated and the residue was dissolved in EtOAc. The mixture was filtered, the filtrate was washed with cold water and brine and concentrated. The residue was purified by reverse phase chromatography on a REDISEP® Gold HP C18 column (275 g) eluting with ACN (from 0% to 100%) in an aq. solution of HCOOH (0.1%), yielding the title compounds (E,Z)-5-(3,5-dimethoxy-4-(2-methoxyvinyl)phenyl)-3,4-dimethyl-1-propylpyridin-2(1H)-one, Int 52-3, as an oil (890 mg).

Method LC-MS_MLG2: Rt=0.98 and 1.14 min; [M+H]⁺=358.

### Step 3: 2-(4-(4,5-dimethyl-6-oxo-1-propyl-1,6-dihydropyridin-3-yl)-2,6-dimethoxyphenyl)acetaldehyde (Intermediate 52)

To a solution of (E,Z)-5-(3,5-dimethoxy-4-(2-methoxyvinyl)phenyl)-3,4-dimethyl-1-propylpyridin-2(1H)-one (Int 52-3, 890 mg, 2.365 mmol) in acetone (20 ml) was added an aq. solution of HCl (2 M, 9.46 ml, 18.92 mmol) and the RM was stirred at 65° C. for 0.5 h. The mixture was concentrated and the residue was purified by chromatography on silica gel eluting with EtOAc (from 0% to 100%) in heptane, yielding the title compound 2-(4-(4,5-dimethyl-6-oxo-1-propyl-1,6-dihydropyridin-3-yl)-2,6-dimethoxyphenyl)acetaldehyde, Intermediate 52, as a solid (426.5 mg).

Method LC-MS_MLG2: Rt=0.96 min; [M+H]⁺=344.

### Intermediate 53: 2,6-difluoro-4-(2-methyl-1-oxo-1,2-dihydro-2,7-naphthyridin-4-yl)benzaldehyde

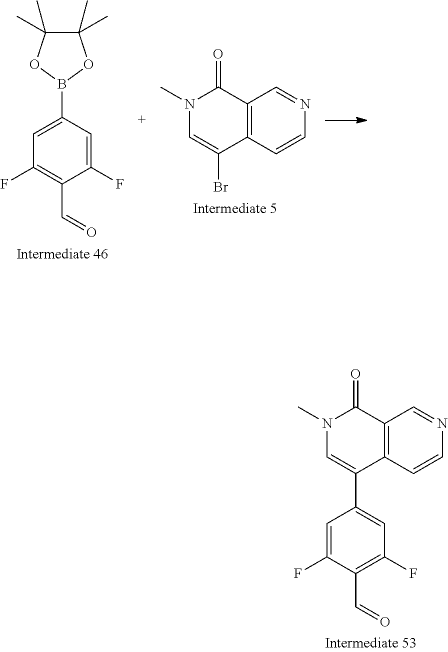 [SMILES: CC1(C)OB(c2cc(F)c(C=O)c(F)c2)OC1(C)C ; Cn1cc(Br)c2ccncc2c1=O ; Cn1cc(-c2cc(F)c(C=O)c(F)c2)c2ccncc2c1=O]

To a mixture of 4-bromo-2-methyl-2,7-naphthyridin-1(2H)-one (Intermediate 5, 250 mg, 0.993 mmol), 2,6-difluoro-4-(4,4,5,5-tetramethyl-1,3,2-dioxaborolan-2-yl)benzaldehyde (Intermediate 46, 336 mg, 1.192 mmol) and Na₂CO₃ (316 mg, 2.98 mmol) in a mixture of 1,4-dioxane (6.0 ml) and water (1.5 ml) under an argon atmosphere was added PdCl₂(dppf)-CH₂Cl₂ (73 mg, 0.099 mmol) and the RM was heated at 100° C. for 2 h. The mixture was cooled to RT and filtered through CELITE® and the solids were washed with EtOAc, MeOH and DCM. The combined filtrates were concentrated and the residue was purified by reverse phase chromatography on a REDISEP® C18 column eluting with ACN (from 2% to 100%) in an aq. solution of TFA (0.1%), yielding the title compound 2,6-difluoro-4-(2-methyl-1-oxo-1,2-dihydro-2,7-naphthyridin-4-yl)benzaldehyde, Intermediate 53, as a solid (92 mg).

Method L CMS_MLG2: Rt=0.53; [M+H]⁺=301.

### Intermediate 54: 4-(2-butyl-1-oxo-1,2-dihydro-2,7-naphthyridin-4-yl)benzaldehyde

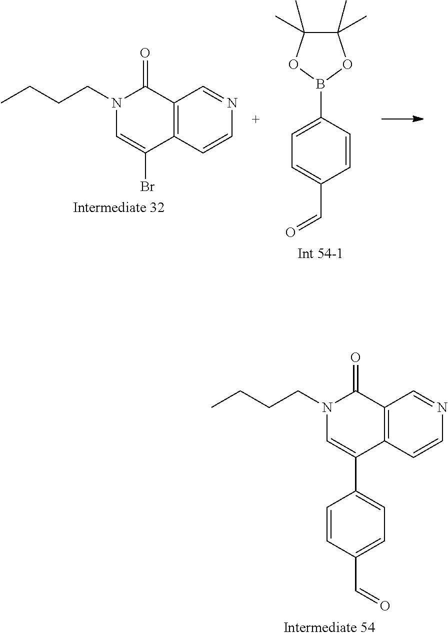

To a mixture of 4-bromo-2-butyl-2,7-naphthyridin-1(2H)-one (Intermediate 32, 300 mg, 1.067 mmol), 4-(4,4,5,5-tetramethyl-1,3,2-dioxaborolan-2-yl)benzaldehyde (Int 54-1, 300 mg, 1.293 mmol) and Na₂CO₃ (339 mg, 3.20 mmol) in a mixture of 1,4-dioxane (8 ml) and water (2 ml) under an argon atmosphere was added PdCl₂(dppf)-CH₂Cl₂(79 mg, 0.107 mmol) and the RM was heated at 100° C. for 2 h. The mixture was filtered through CELITE®, the solids were washed with EtOAc and the combined filtrates were concentrated. Et₂O was added to the residue, the mixture was filtered and the solids were concentrated, yielding the title compound 4-(2-butyl-1-oxo-1,2-dihydro-2,7-naphthyridin-4-yl)benzaldehyde, Intermediate 54, as a solid (269 mg). The filtrate was concentrated and the residue was purified by reverse phase chromatography on a REDISEP©C18 column (15.5 g) eluting with ACN (from 1% to 100%) in an aq. solution of TFA (0.1%), yielding the title compound 4-(2-butyl-1-oxo-1,2-dihydro-2,7-naphthyridin-4-yl)benzaldehyde, Intermediate 54, as a solid (66 mg).

Method LCMS_MLG8: Rt=0.87 min; [M+H]⁺=307.

### Intermediate 55: 4-(2-butyl-1-oxo-1,2-dihydro-2,7-naphthyridin-4-yl)-2,6-difluorobenzaldehyde

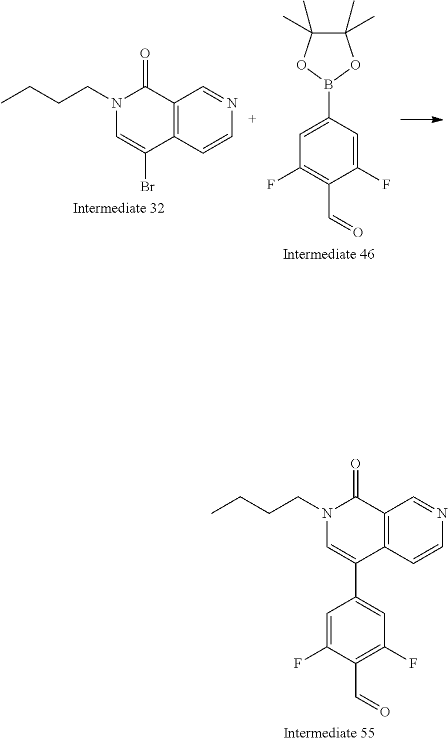 [SMILES: CCCCn1cc(Br)c2ccncc2c1=O ; CC1(C)OB(c2cc(F)c(C=O)c(F)c2)OC1(C)C ; CCCCn1cc(-c2cc(F)c(C=O)c(F)c2)c2ccncc2c1=O]

To a mixture of 4-bromo-2-butyl-2,7-naphthyridin-1(2H)-one (Intermediate 32, 300 mg, 1.067 mmol), 2,6-difluoro-4-(4,4,5,5-tetramethyl-1,3,2-dioxaborolan-2-yl)benzaldehyde (Intermediate 46, 350 mg, 1.240 mmol) and Na₂CO₃ (339 mg, 3.20 mmol) in a mixture of 1,4-dioxane (8 ml) and water (2 ml) under an argon atmosphere was added PdCl₂(dppf)-CH₂Cl₂ (79 mg, 0.107 mmol) and the RM was heated at 100° C. for 2 h. The mixture was filtered over CELITE® and the solids were washed with EtOAc. The filtrate was concentrated and the residue was purified by reverse phase chromatography on a REDISEP® C18 column (15.5 g) eluting with ACN (1% to 100%) in an aq. solution of TFA (0.1%), yielding the title compound 4-(2-butyl-1-oxo-1,2-dihydro-2,7-naphthyridin-4-yl)-2,6-difluorobenzaldehyde, Intermediate 55, as a solid (368 mg).

Method LCMS_MLG8: Rt=0.91 min; [M+H]⁺=343.

¹H NMR (400 MHz, DMSO-d₆) δ[ppm] 10.28 (s, 1H), 9.47 (d, J=0.8 Hz, 1H), 8.77 (d, J=5.8 Hz, 1H), 8.06 (s, 1H), 7.61 (dd, J=5.7, 0.9 Hz, 1H), 7.52-7.44 (m, 2H), 4.05 (t, J=7.4 Hz, 2H), 1.72 (m, 2H), 1.34 (m, 2H), 0.93 (t, J=7.4 Hz, 3H).

### Intermediate 56: 4-(2-butyl-1-oxo-1,2-dihydro-2,7-naphthyridin-4-yl)-2-fluoro-6-methoxybenzaldehyde

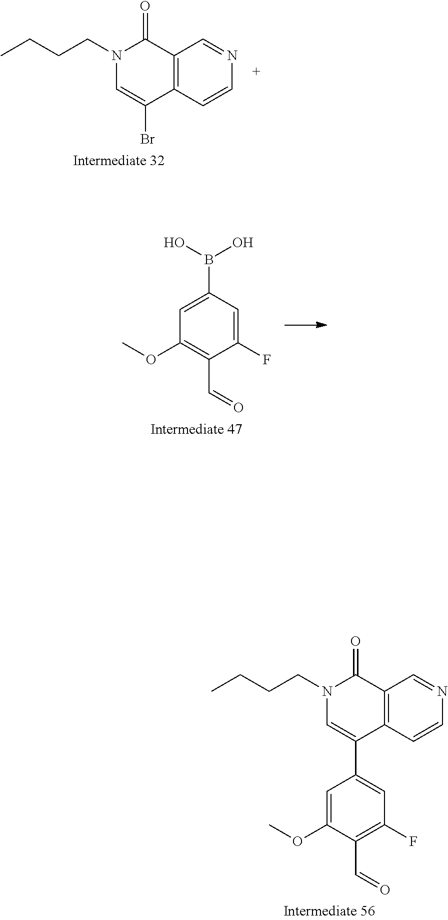 [SMILES: CCCCn1cc(Br)c2ccncc2c1=O ; COc1cc(B(O)O)cc(F)c1C=O ; CCCCn1cc(-c2cc(F)c(C=O)c(OC)c2)c2ccncc2c1=O]

To a mixture of 4-bromo-2-butyl-2,7-naphthyridin-1(2H)-one (Intermediate 32, 400 mg, 1.067 mmol), (3-fluoro-4-formyl-5-methoxyphenyl)boronic Acid (Intermediate 47, 277 mg, 1.174 mmol) and Na₂CO₃ (339 mg, 3.20 mmol) in a mixture of 1,4-dioxane (6 ml) and water (1.5 ml) under an argon atmosphere was added PdCl₂(dppf)-CH₂Cl₂ (39 mg, 0.053 mmol) and the RM was heated at 100° C. for 16 h. The mixture was cooled to RT, filtered over CELITE® and the solids were washed with 1,4-dioxane. The filtrate was concentrated and the residue was purified by reverse phase chromatography on a REDISEP® C18 column (15.5 g) eluting with ACN (1% to 100%) in an aq. solution of TFA (0.1%), yielding the title compound 4-(2-butyl-1-oxo-1,2-dihydro-2,7-naphthyridin-4-yl)-2-fluoro-6-methoxybenzaldehyde, Intermediate 56, as a solid (266 mg).

Method LCMS_MLG1: Rt=0.93 min; [M+H]⁺=355.

### Intermediate 57: 2-(4-(2-butyl-1-oxo-1,2-dihydro-2,7-naphthyridin-4-yl)-2,5-dimethoxyphenyl)acetaldehyde

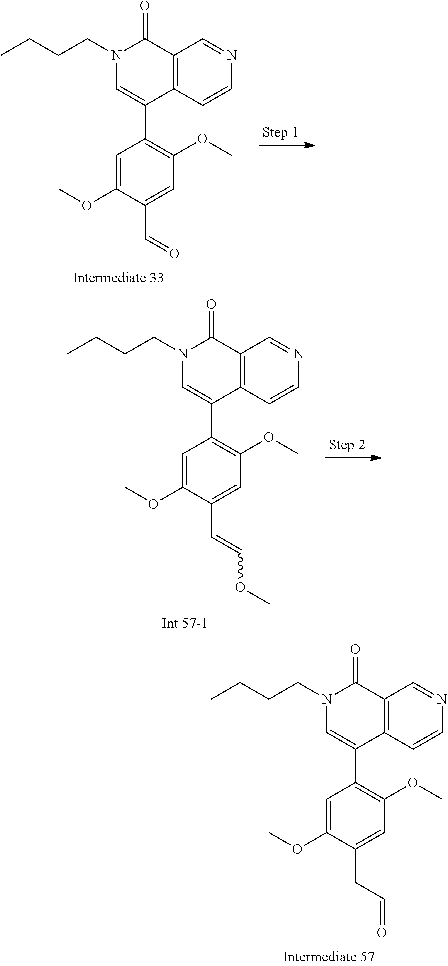 [SMILES: CCCCn1cc(-c2cc(OC)c(C=O)cc2OC)c2ccncc2c1=O ; CCCCn1cc(-c2cc(OC)c(C=COC)cc2OC)c2ccncc2c1=O ; CCCCn1cc(-c2cc(OC)c(CC=O)cc2OC)c2ccncc2c1=O]

### Step 1: (E,Z)-2-butyl-4-(2,5-dimethoxy-4-(2-methoxyvinyl)phenyl)-2,7-naphthyridin-1(2H)-one (Int 57-1)

To a solution of (methoxymethyl)triphenylphosphonium chloride (950 mg, 2.77 mmol) in THE (9 ml) at 0° C. was added a solution of t-BuOK (1 M) in THE (3.7 ml, 3.70 mmol) and the RM was stirred at 0° C. for 30 min. 4-(2-Butyl-1-oxo-1,2-dihydro-2,7-naphthyridin-4-yl)-2,5-dimethoxybenzaldehyde (Intermediate 33, 360 mg, 0.924 mmol) was added and the RM was stirred at RT for 1 h, then at 70° C. for 1 h. The mixture was cooled to RT, added into water and the aq. phase was extracted with EtOAc. The organic phase was washed with water and brine, dried over MgSO₄ and concentrated. The residue was purified by chromatography on silica gel eluting with EtOAc (from 0% to 100%) in CHX, yielding the title compounds (E,Z)-2-butyl-4-(2,5-dimethoxy-4-(2-methoxyvinyl)phenyl)-2,7-naphthyridin-1(2H)-one, Int 57-1, as a solid (153 mg).

Method LCMS_MLG8: Rt=1.06/1.09 min; [M+H]⁺=395.

### Step 2: 2-(4-(2-butyl-1-oxo-1,2-dihydro-2,7-naphthyridin-4-yl)-2,5-dimethoxyphenyl)acetaldehyde (Int 57)

To a solution of (E,Z)-2-butyl-4-(2,5-dimethoxy-4-(2-methoxyvinyl)phenyl)-2,7-naphthyridin-1(2H)-one (Int 57-1, 153 mg, 0.326 mmol) in acetone (3 ml) was added an aq. solution of HCl (2 M, 1.4 ml, 2.8 mmol) and the RM was stirred at 65° C. for 1 h. The mixture was concentrated and the residue was purified by reverse phase chromatography on a REDISEP® C18 column eluting with ACN (from 1% to 100%) in an aq. solution of TFA (0.1%), yielding the title compound 2-(4-(2-butyl-1-oxo-1,2-dihydro-2,7-naphthyridin-4-yl)-2,5-dimethoxyphenyl)acetaldehyde, Intermediate 57, as a solid (104 mg).

Method LCMS_MLG8: Rt=0.84 min; [M+H]⁺=381.

### Intermediate 58: 2-butyl-4-(3-fluoro-4-hydroxyphenyl)-2,7-naphthyridin-1(2H)-one

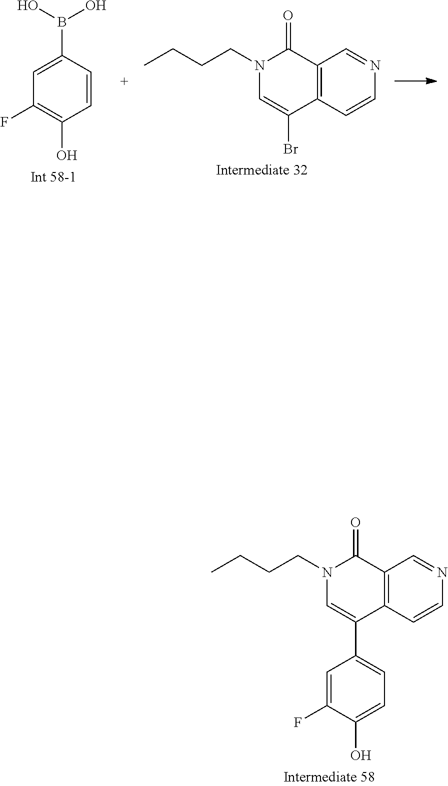 [SMILES: OB(O)c1ccc(O)c(F)c1 ; CCCCn1cc(Br)c2ccncc2c1=O ; CCCCn1cc(-c2ccc(O)c(F)c2)c2ccncc2c1=O]

To a mixture of 4-bromo-2-butyl-2,7-naphthyridin-1(2H)-one (Intermediate 32, 300 mg, 1.067 mmol), 3-fluoro-4-hydroxyphenylboronic Acid (Int 58-1, 200 mg, 1.280 mmol) and Na₂CO₃ (339 mg, 3.20 mmol) in a mixture of 1,4-dioxane (8 ml) and water (2 ml) under an argon atmosphere was added PdCl₂(dppf)-CH₂Cl₂ (79 mg, 0.107 mmol) and the RM was heated at 100° C. for 2 h. The mixture was filtered through CELITE®, the solids were washed with EtOAc and the combined filtrates were concentrated. The residue was purified by reverse phase chromatography on a REDISEP® Gold HP C18 column (15.5 g) eluting with ACN (from 1% to 100%) in an aq. solution of TFA (0.1%), yielding the title compound 2-butyl-4-(3-fluoro-4-hydroxyphenyl)-2,7-naphthyridin-1(2H)-one, Intermediate 58, as a solid (154 mg).

Method LCMS_MLG8: Rt=0.86 min; [M+H]⁺=313.

### Intermediate 59: 2-butyl-4-(3,5-difluoro-4-hydroxyphenyl)-2,7-naphthyridin-1(2H)-one

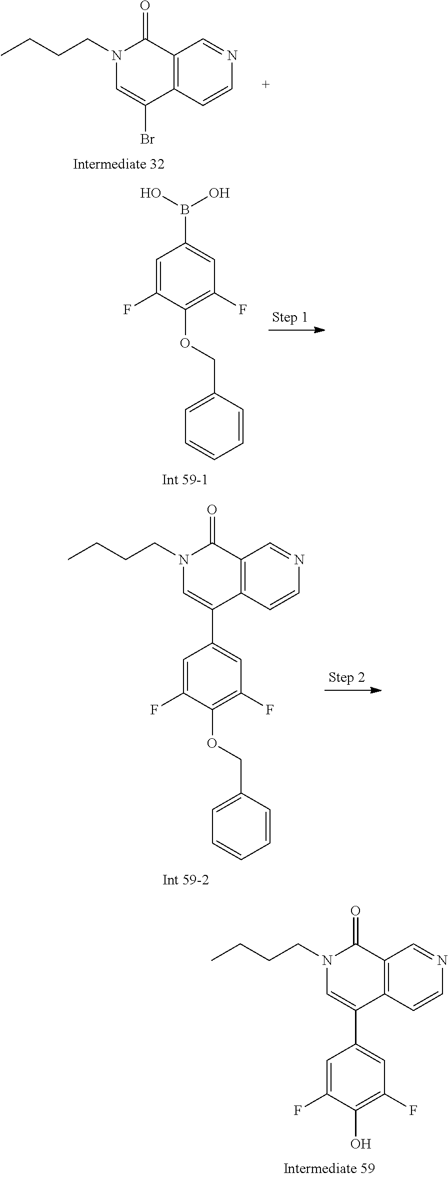 [SMILES: CCCCn1cc(Br)c2ccncc2c1=O ; OB(O)c1cc(F)c(OCc2ccccc2)c(F)c1 ; CCCCn1cc(-c2cc(F)c(OCc3ccccc3)c(F)c2)c2ccncc2c1=O ; CCCCn1cc(-c2cc(F)c(O)c(F)c2)c2ccncc2c1=O]

### Step 1: 4-(4-(benzyloxy)-3,5-difluorophenyl)-2-butyl-2,7-naphthyridin-1(2H)-one (Int 59-2)

To a mixture of 4-bromo-2-butyl-2,7-naphthyridin-1(2H)-one (Intermediate 32, 300 mg, 1.067 mmol), 4-benzyloxy-3,5-difluorophenylboronic acid (Int 59-1, 349 mg, 1.280 mmol) and Na₂CO₃ (339 mg, 3.20 mmol) in a mixture of 1,4-dioxane (8 ml) and water (2 ml) under an argon atmosphere was added PdCl₂(dppf)-CH₂Cl₂ (79 mg, 0.107 mmol) and the RM was heated in a microwave oven at 100° C. for 30 min. The mixture was filtered through CELITE®, the solids were washed with EtOAc and the filtrate was concentrated. The residue was dissolved in a mixture of DCM and water and the aq. phase was extracted with EtOAc. The combined organic phases were washed with brine, dried over MgSO₄ and concentrated. The residue was purified by chromatography on silica gel eluting with a mixture (4:1) of EtOAc and MeOH (from 0% to 100%) in DCM, yielding the title compound 4-(4-(benzyloxy)-3,5-difluorophenyl)-2-butyl-2,7-naphthyridin-1(2H)-one, Int 59-2, as a solid (485 mg).

Method LCMS_MLG9: Rt=1.05 min; [M+H]⁺=421.

### Step 2: 2-butyl-4-(3,5-difluoro-4-hydroxyphenyl)-2,7-naphthyridin-1(2H)-one (Intermediate 59)

To a solution of 4-(4-(benzyloxy)-3,5-difluorophenyl)-2-butyl-2,7-naphthyridin-1(2H)-one (Int 59-2, 480 mg, 1.016 mmol) in MeOH (10 ml) under an argon atmosphere was added Pd/C (10%, 100 mg, 0.094 mmol) and the RM was vigorously stirred under a hydrogen atmosphere at RT for 20 h. The mixture was filtered through CELITE®, the solids were washed with MeOH and the filtrate was concentrated. EtOAc was added to the residue and the mixture was sonicated for 5 min. The mixture was filtered and the solids were dried, yielding the title compound 2-butyl-4-(3,5-difluoro-4-hydroxyphenyl)-2,7-naphthyridin-1(2H)-one, Intermediate 59, as a solid (219 mg).

Method LCM_MLG9: Rt=0.69 min; [M+H]⁺=331.

### Intermediate 60: 4-(2,5-dimethoxy-4-((4-(piperidin-4-yloxy)piperidin-1-yl)methyl)phenyl)-2-methyl-2,7-naphthyridin-1(2H)-one

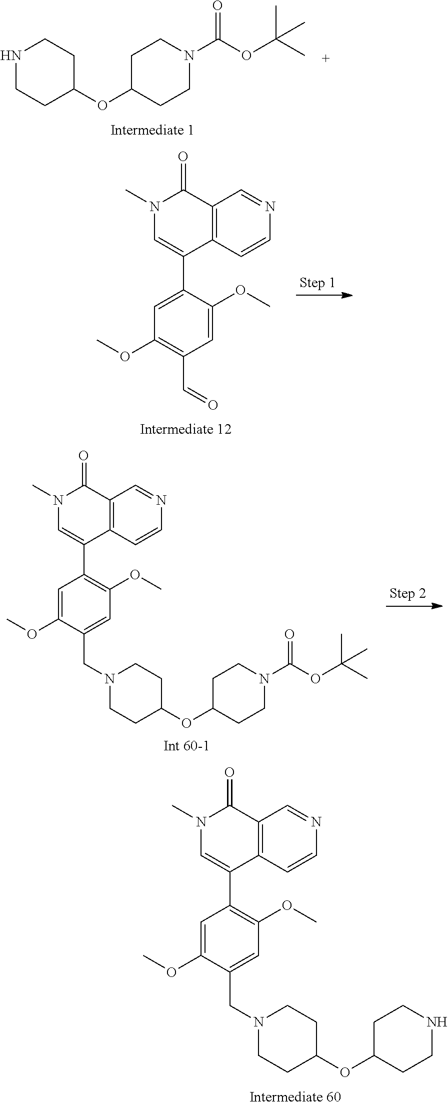

### Step 1: tert-butyl 4-((1-(2,5-dimethoxy-4-(2-methyl-1-oxo-1,2-dihydro-2,7-naphthyridin-4-yl)benzyl)piperidin-4-yl)oxy)piperidine-1-carboxylate (Int 60-1)

To a suspension of tert-butyl 4-(piperidin-4-yloxy)piperidine-1-carboxylate (Intermediate 1, 156 mg, 0.55 mmol) in DCM (0.5 ml) under an argon atmosphere were added NaOAc (18.4 mg, 0.22 mmol), HOAc (12 ul, 0.20 mmol) and a solution of 2,5-dimethoxy-4-(2-methyl-1-oxo-1,2-dihydro-2,7-naphthyridin-4-yl)benzaldehyde (Intermediate 12, 66 mg, 0.20 mmol) in DCM (1.5 ml). The RM was stirred at RT for 15 min, solid NaBH(OAc)₃ (86 mg, 0.407 mmol) was added and the RM was stirred at RT for 22.5 h. A sat. aq. solution of NaHCO₃ was added and the mixture was extracted with DCM. The combined organic phases were dried over MgSO₄, and the residue was purified by chromatography on silica gel eluting with MeOH (from 0% to 20%) in DCM, yielding the title compound tert-butyl 4-((1-(2,5-dimethoxy-4-(2-methyl-1-oxo-1,2-dihydro-2,7-naphthyridin-4-yl)benzyl)piperidin-4-yl)oxy)piperidine-1-carboxylate, Int I60-1, as a solid (107 mg).

Method LCMS_MLG1: Rt=0.78 min; [M+H]⁺=593.

### Step 2: 4-(2,5-dimethoxy-4-((4-(piperidin-4-yloxy)piperidin-1-yl)methyl)phenyl)-2-methyl-2,7-naphthyridin-1(2H)-one (Intermediate 60)

To a solution of tert-butyl 4-((1-(2,5-dimethoxy-4-(2-methyl-1-oxo-1,2-dihydro-2,7-naphthyridin-4-yl)benzyl)piperidin-4-yl)oxy)piperidine-1-carboxylate (Int 60-1, 107 mg, 0,181 mmol) in DCM (1 ml) was added TFA (1 ml). The RM was stirred at RT for 15 min, the mixture was concentrated, the residue was dissolved in MeOH and applied on Biotage® ISOLUTE® SCX SPE (2 g) cartridge. After eluting with MeOH and discarding the filtrate, a solution of ammonia (7 M) in MeOH was passed through the cartridge and the combined filtrates were concentrated, yielding the title compound 4-(2,5-dimethoxy-4-((4-(piperidin-4-yloxy)piperidin-1-yl)methyl)phenyl)-2-methyl-2,7-naphthyridin-1(2H)-one, Intermediate 60, as an oil (83 mg).

Method LCMS_MLG1: Rt=0.35 min; [M+H]⁺=493.

### Intermediate 61: 4-(4-((4-(((3R,4R)-3-fluoropiperidin-4-yl)oxy)piperidin-1-yl)methyl)-2,5-dimethoxyphenyl)-2-methyl-2,7-naphthyridin-1(2H)-one

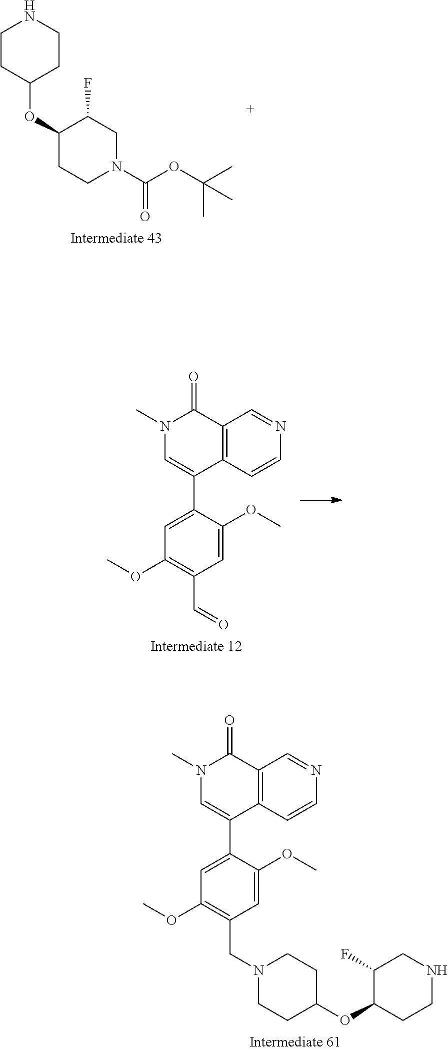 [SMILES: CC(C)(C)OC(=O)N1CC[C@@H](OC2CCNCC2)[C@H](F)C1 ; COc1cc(-c2cn(C)c(=O)c3cnccc23)c(OC)cc1C=O ; COc1cc(-c2cn(C)c(=O)c3cnccc23)c(OC)cc1CN1CCC(O[C@@H]2CCNC[C@H]2F)CC1]

To a mixture of 2,5-dimethoxy-4-(2-methyl-1-oxo-1,2-dihydro-2,7-naphthyridin-4-yl)benzaldehyde (Intermediate 12, 800 mg, 2.47 mmol) and tert-butyl (3R,4R)-3-fluoro-4-(piperidin-4-yloxy)piperidine-1-carboxylate 4-methylbenzenesulfonate salt (Intermediate 43, 1.288 g, 2.71 mmol) in a mixture of DCM (27 ml) and DMF (9 ml) under an argon atmosphere were added NaOAc (809 mg, 9.87 mmol), HOAc (635 μl, 11.10 mmol) and NaBH(OAc)₃ (1.046 g, 4.93 mmol) and the RM was stirred at RT for 4.5 h. A sat. aq. solution of NaHCO₃ was added, the aq. phase was extracted with DCM, the organic phase was washed with an aq. solution of LiBr (0.1 M) and brine, dried over MgSO₄ and concentrated. The residue was purified by chromatography on silica gel eluting with MeOH (from 0% to 20%) in DCM. Fractions containing the title compound were combined and concentrated. The residue was taken up in DCM (5 ml), TFA (2 ml) was added and the RM was stirred at RT for 20 min. The mixture was concentrated, the residue was dissolved in a mixture of ACN and water, adsorbed on ISOLUTE® HM-N and purified by reverse phase chromatography on a REDISEP® Gold C18 column eluting with ACN (from 2% to 100%) in an aq. solution of TFA (0.1%), yielding the title compound 4-(4-((4-(((3R,4R)-3-fluoropiperidin-4-yl)oxy)piperidin-1-yl)methyl)-2,5-dimethoxyphenyl)-2-methyl-2,7-naphthyridin-1(2H)-one, Intermediate 61, as a solid TFA salt (680 mg).

Method LCMS_JL2: Rt=0.53 min; [M+H]⁺=511.

### Intermediate 62: 4-(3,5-dimethoxy-4-(2-(4-(piperidin-4-yloxy)piperidin-1-yl)ethyl)phenyl)-2-hexyl-2,7-naphthyridin-1(2H)-one

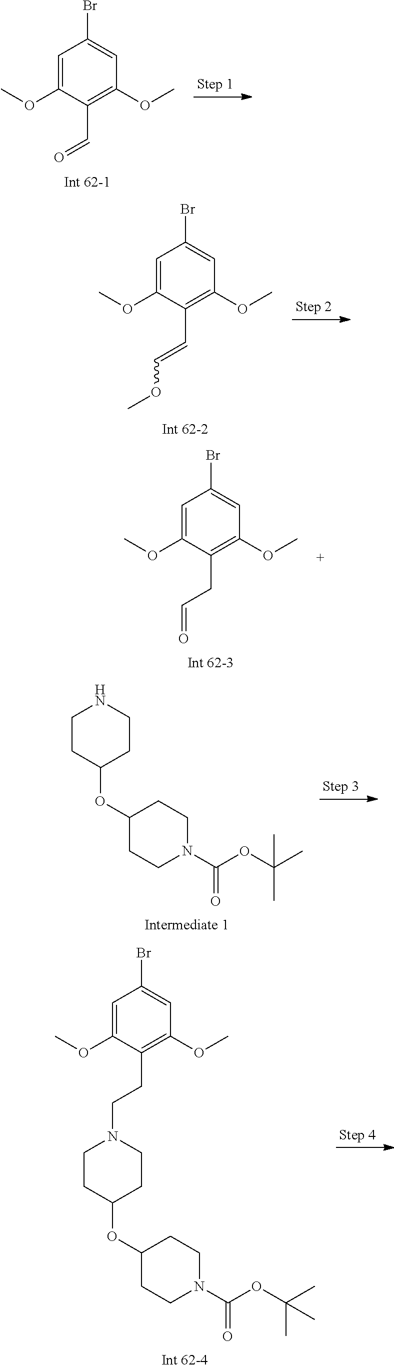

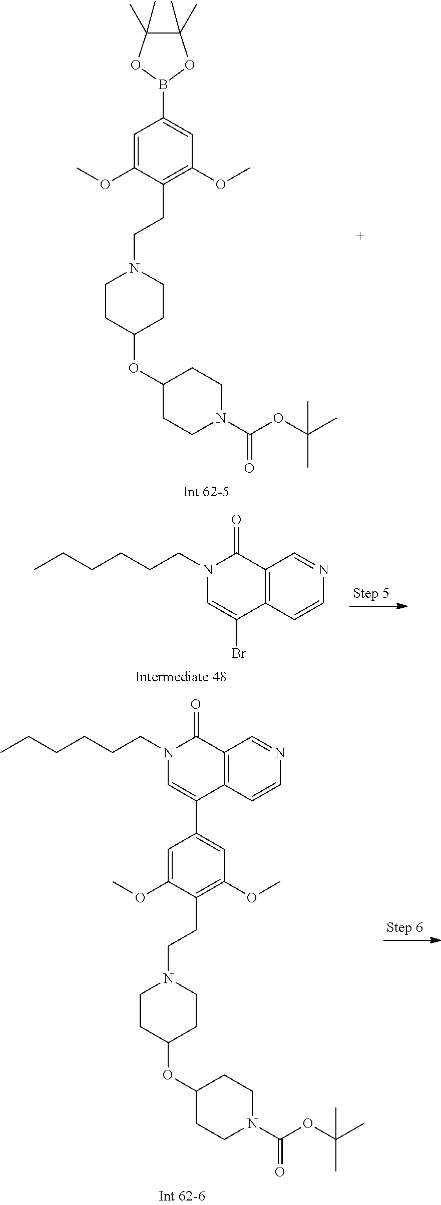

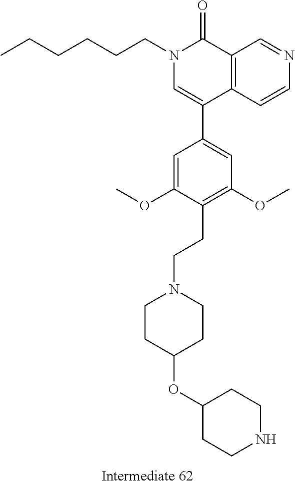 [SMILES: CCCCCCn1cc(-c2cc(OC)c(CCN3CCC(OC4CCNCC4)CC3)c(OC)c2)c2ccncc2c1=O]

### Step 1: (E,Z)-5-bromo-1,3-dimethoxy-2-(2-methoxyvinyl)benzene (Int 62-2)

To a solution of methoxymethyl-triphenylphosphonium chloride (6.29 g, 18.36 mmol) in THE (60 ml) was added a solution of t-BuOK (1 M) in TH (24 ml, 24.00 mmol) and the RM was stirred at 0° C. for 30 min. 4-Bromo-2,6-dimethoxybenzaldehyde (Int 62-1, 1.5 g, 6.12 mmol) was added and the RM was stirred at 0° C. for 2 h and at 70° C. for 18 h. The mixture was added into water and the aq. phase was extracted with EtOAc. The organic phase was washed with water and brine, dried over MgSO₄ and concentrated. The residue was purified by chromatography on silica gel eluting with EtOAc (from 0% to 50%) in CHX, yielding the title compounds (E,Z)-5-bromo-1,3-dimethoxy-2-(2-methoxyvinyl)benzene, Int 62-2, as a solid (1.57 g).

Method LCMS_MLG1: Rt=1.11 min; [M+H]⁺=273 and 275.

### Step 2: 2-(4-bromo-2,6-dimethoxyphenyl)acetaldehyde (Int 62-3)

To a solution of 5-bromo-1,3-dimethoxy-2-(2-methoxyvinyl)benzene (Int 62-2, 1.564 g, 5.55 mmol) in acetone (50 ml) was added an aq. solution of HCl (2 M, 11 ml, 22.0 mmol) and the RM was heated at 65° C. for 2 h. The mixture was concentrated and freeze-dried, yielding the title compound 2-(4-bromo-2,6-dimethoxyphenyl)acetaldehyde, Int 62-3, as a solid (1.377 g), which was used for the next step without further purification.

Method LCMS_MLG1: Rt=1.08 min; [M+H]⁺=259 and 261.

### Step 3: tert-butyl 4-((1-(4-bromo-2,6-dimethoxyphenethyl)piperidin-4-yl)oxy)piperidine-1-carboxylate (Int 62-4)

To a solution of 2-(4-bromo-2,6-dimethoxyphenyl)acetaldehyde (Int 62-3, 1.034 g, 3.47 mmol) and tert-butyl 4-(piperidin-4-yloxy)piperidine-1-carboxylate (Intermediate 1, 1.185 g, 4.17 mmol) in DMSO (15 ml) under an argon atmosphere was added a solution of ZnCl₂ (0.5 M) in THE (9 ml, 4.5 mmol) and the RM was stirred at RT for 5 h. Solid NaBH₃CN (436 mg, 6.94 mmol) was added and the RM was stirred at RT for 4 days. The mixture was diluted with water, the aq. phase was extracted with EtOAc, the organic phase was washed with brine, dried over MgSO₄ and concentrated. The residue was purified by reverse phase chromatography on a REDISEP® C18 column (15.5 g) eluting with ACN (0% to 100%) in an aq. solution of TFA (0.1%), yielding the title compound tert-butyl 4-((1-(4-bromo-2,6-dimethoxyphenethyl)piperidin-4-yl)oxy)piperidine-1-carboxylate, Int 62-4, as a solid TFA salt (515 mg).

Method LCMS_MLG8: Rt=0.92 min; [M+H]⁺=527 and 529.

### Step 4: tert-butyl 4-((1-(2,6-dimethoxy-4-(4,4,5,5-tetramethyl-1,3,2-dioxaborolan-2-yl)phenethyl)piperidin-4-yl)oxy)piperidine-1-carboxylate (Int 62-5)

To a mixture of tert-butyl 4-((1-(4-bromo-2,6-dimethoxyphenethyl)piperidin-4-yl)oxy)piperidine-1-carboxylate TFA salt (Int 62-4, 515 mg, 0.713 mmol), BISPIN (271 mg, 1.069 mmol), dppf (12 mg, 0.022 mmol) and KOAc (210 mg, 2.138 mmol) in 1,4-dioxane (7 ml) under an argon atmosphere was added PdCl₂(dppf) (16 mg, 0.022 mmol) and the RM was heated at 90° C. for 20 h. The mixture was filtered through CELITE®, the solids were washed with EtOAc. The filtrate was washed with an aq. solution of HCl (0.1 M) and brine, dried over MgSO₄ and concentrated. The residue was purified by reverse phase chromatography on a REDISEP® C18 column (15.5 g) eluting with ACN (from 1% to 100%) in an aq. solution of TFA (0.1%), yielding the title compound tert-butyl 4-((1-(2,6-dimethoxy-4-(4,4,5,5-tetramethyl-1,3,2-dioxaborolan-2-yl)phenethyl)piperidin-4-yl)oxy)piperidine-1-carboxylate, Int 62-5, as a solid TFA salt (213 mg).

Method LCMS_MLG8: Rt=1.03 min; [M+H]⁺=575.

### Step 5: tert-butyl 4-((1-(4-(2-hexyl-1-oxo-1,2-dihydro-2,7-naphthyridin-4-yl)-2,6-dimethoxyphenethyl)piperidin-4-yl)oxy)piperidine-1-carboxylate (Int 62-6)

To a mixture of 4-bromo-2-hexyl-2,7-naphthyridin-1(2H)-one (Intermediate 48, 100 mg, 0.307 mmol), solid K₂CO₃ (127 mg, 0.919 mmol) and tert-butyl 4-((1-(2,6-dimethoxy-4-(4,4,5,5-tetramethyl-1,3,2-dioxaborolan-2-yl)phenethyl)piperidin-4-yl)oxy)piperidine-1-carboxylate TFA salt (Int 62-5, 138 mg, 0.200 mmol) in a mixture of ACN (4 ml) and water (1 ml) under an argon atmosphere was added PdCl₂(dppf) (12 mg, 0.016 mmol) and the RM was heated at 100° C. for 1 h. The mixture was filtered through CELITE® and the solids were washed with ACN. The filtrate was concentrated and the residue was purified by reverse phase chromatography on a REDISEP® C18 column (15.5 g) eluting with ACN (from 1% to 100%) in an aq. solution of TFA (0.1%), yielding the title compound tert-butyl 4-((1-(4-(2-hexyl-1-oxo-1,2-dihydro-2,7-naphthyridin-4-yl)-2,6-dimethoxyphenethyl)piperidin-4-yl)oxy)piperidine-1-carboxylate, Int 62-6, as a solid TFA salt (176 mg).

Method LCMS_MLG8: Rt=1.02 min; [M+H]⁺=678.

### Step 6: 4-(3,5-dimethoxy-4-(2-(4-(piperidin-4-yloxy)piperidin-1-yl)ethyl)phenyl)-2-hexyl-2,7-naphthyridin-1(2H)-one (Intermediate 62)

To a solution of tert-butyl 4-((1-(4-(2-hexyl-1-oxo-1,2-dihydro-2,7-naphthyridin-4-yl)-2,6-dimethoxyphenethyl)piperidin-4-yl)oxy)piperidine-1-carboxylate TFA salt (Int 62-6, 176 mg, 0.205 mmol) in DCM (2 ml) was added TFA (0.450 ml, 5.84 mmol) and the RM was stirred at RT for 1 h. The mixture was concentrated and the residue was purified by reverse phase chromatography on a REDISEP® C18 column (15.5 g) eluting with ACN (from 1% to 100%) in an aq. solution of TFA (0.1%), yielding the title compound 4-(3,5-dimethoxy-4-(2-(4-(piperidin-4-yloxy)piperidin-1-yl)ethyl)phenyl)-2-hexyl-2,7-naphthyridin-1(2H)-one, Intermediate 62, as a solid TFA salt (97 mg).

Method LCMS_MLG8: Rt=0.64 min; [M+H]⁺=577.

### Intermediate 63: 2-butyl-4-(3,5-difluoro-4-(piperidin-4-yloxy)phenyl)-2,7-naphthyridin-1(2H)-one

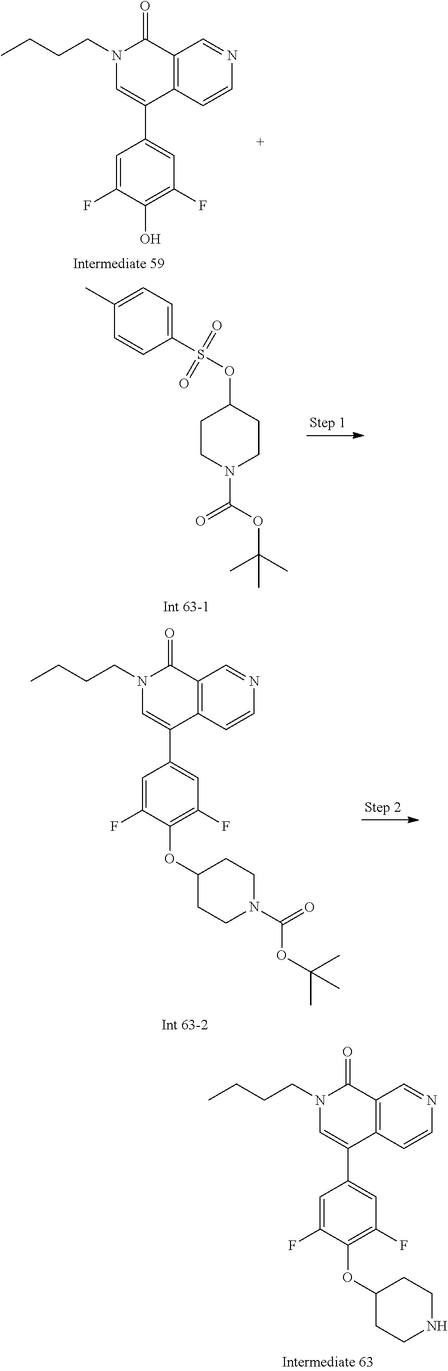

### Step 1: tert-butyl 4-(4-(2-butyl-1-oxo-1,2-dihydro-2,7-naphthyridin-4-yl)-2,6-difluorophenoxy)piperidine-1-carboxylate (Int 63-2)

To a mixture of 2-butyl-4-(3,5-difluoro-4-hydroxyphenyl)-2,7-naphthyridin-1(2H)-one (Intermediate 59, 110 mg, 0.333 mmol) and solid Cs₂CO₃ (219 mg, 0.666 mmol) in DMF (3 ml) was added tert-butyl 4-(tosyloxy)piperidine-1-carboxylate (Int 63-1, 237 mg, 0.666 mmol) and the RM was stirred at 100° C. for 2 h. The mixture was purified by reverse phase chromatography on a REDISEP® C18 column (15.5 g) eluting with ACN (from 1% to 100%) in an aq. solution of TFA (0.1%), yielding the title compound tert-butyl 4-(4-(2-butyl-1-oxo-1,2-dihydro-2,7-naphthyridin-4-yl)-2,6-difluorophenoxy)piperidine-1-carboxylate, Int 63-2, as a solid (189 mg).

Method LCMS_MLG9: Rt=1.12 min; [M+H]⁺=514.

### Step 2: 2-butyl-4-(3,5-difluoro-4-(piperidin-4-yloxy)phenyl)-2,7-naphthyridin-1(2H)-one (Intermediate 63)

To a solution of tert-butyl 4-(4-(2-butyl-1-oxo-1,2-dihydro-2,7-naphthyridin-4-yl)-2,6-difluorophenoxy)piperidine-1-carboxylate (Int 63-2, 185 mg, 0.324 mmol) in DCM (3 ml) was added TFA (0.800 ml, 10.38 mmol) and the RM was stirred at RT for 1 h. The mixture was concentrated, yielding the title compound 2-butyl-4-(3,5-difluoro-4-(piperidin-4-yloxy)phenyl)-2,7-naphthyridin-1(2H)-one, Intermediate 63, as a solid TFA salt (176 mg).

Method LCMS_MLG9: Rt=0.59 min; [M+H]⁺=414.

### Intermediate 64: 2-butyl-4-(3,5-difluoro-4-(((3R,4R)-3-fluoropiperidin-4-yl)oxy)phenyl)-2,7-naphthyridin-1(2H)-one

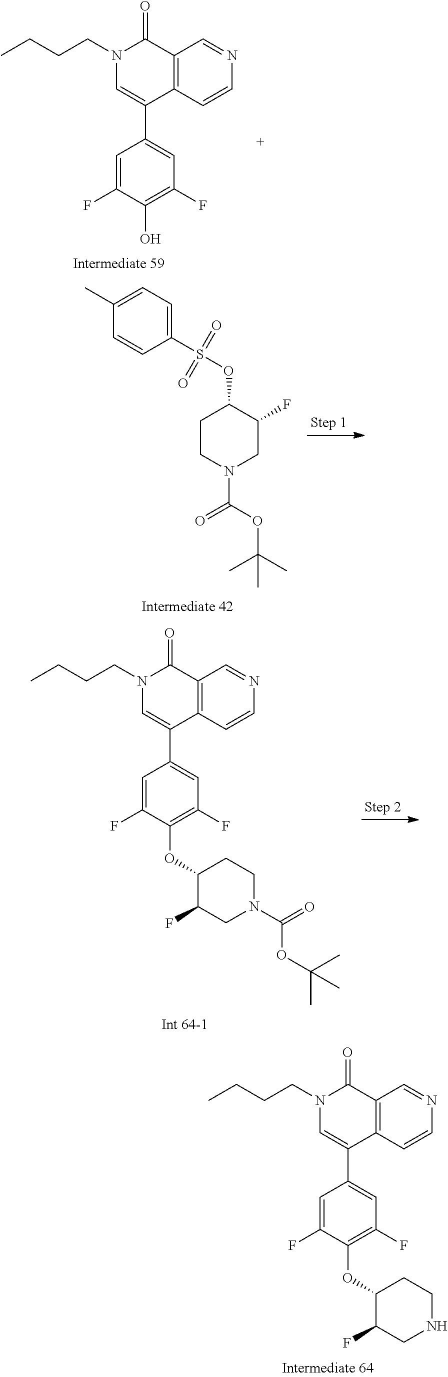

### Step 1: tert-butyl (3R,4R)-4-(4-(2-butyl-1-oxo-1,2-dihydro-2,7-naphthyridin-4-yl)-2,6-difluorophenoxy)-3-fluoropiperidine-1-carboxylate (Int 64-1)

To a mixture of 2-butyl-4-(3,5-difluoro-4-hydroxyphenyl)-2,7-naphthyridin-1(2H)-one (Intermediate 59, 110 mg, 0.333 mmol) and solid Cs₂CO₃ (219 mg, 0.666 mmol) in DMF (3 ml) was added tert-butyl (3R,4S)-3-fluoro-4-(tosyloxy)piperidine-1-carboxylate (Intermediate 42, 249 mg, 0.666 mmol) at RT and the RM was stirred at 100° C. for 2 h. The mixture was added into water and the aq. phase was extracted with EtOAc. The organic phase was washed with brine, dried over MgSO₄ and concentrated, yielding the title compound tert-butyl (3R,4R)-4-(4-(2-butyl-1-oxo-1,2-dihydro-2,7-naphthyridin-4-yl)-2,6-difluorophenoxy)-3-fluoropiperidine-1-carboxylate, Int 64-1, as an oil (239 mg), which was used for the next step without further purification.

Method LCMS_MLG9: Rt=1.06 min; [M+H]⁺=532.

### Step 2: 2-butyl-4-(3,5-difluoro-4-(((3R,4R)-3-fluoropiperidin-4-yl)oxy)phenyl)-2,7-naphthyridin-1(2H)-one (Intermediate 64)

To a solution of tert-butyl (3R,4R)-4-(4-(2-butyl-1-oxo-1,2-dihydro-2,7-naphthyridin-4-yl)-2,6-difluorophenoxy)-3-fluoropiperidine-1-carboxylate (Int 64-1, 177 mg, 0.333 mmol) in DCM (3 ml) was added TFA (0.80 ml, 10.38 mmol) and the RM was stirred at RT for 1 h. The mixture was concentrated and the residue was purified by reverse phase chromatography on a REDISEP® C18 column (15.5 g) eluting with ACN (from 1% to 100%) in an aq. solution of TFA (0.1%), yielding the title compound 2-butyl-4-(3,5-difluoro-4-(((3R,4R)-3-fluoropiperidin-4-yl)oxy)phenyl)-2,7-naphthyridin-1(2H)-one, Intermediate 64, as a solid TFA salt (436 mg).

Method LCMS_MLG9: Rt=0.59 min; [M+H]⁺=432.

### Intermediate 65: 2-butyl-4-(4-(((3S,4S)-3-fluoro-1-(piperidin-4-ylmethyl)piperidin-4-yl)oxy)phenyl)-2,7-naphthyridin-1(2H)-one

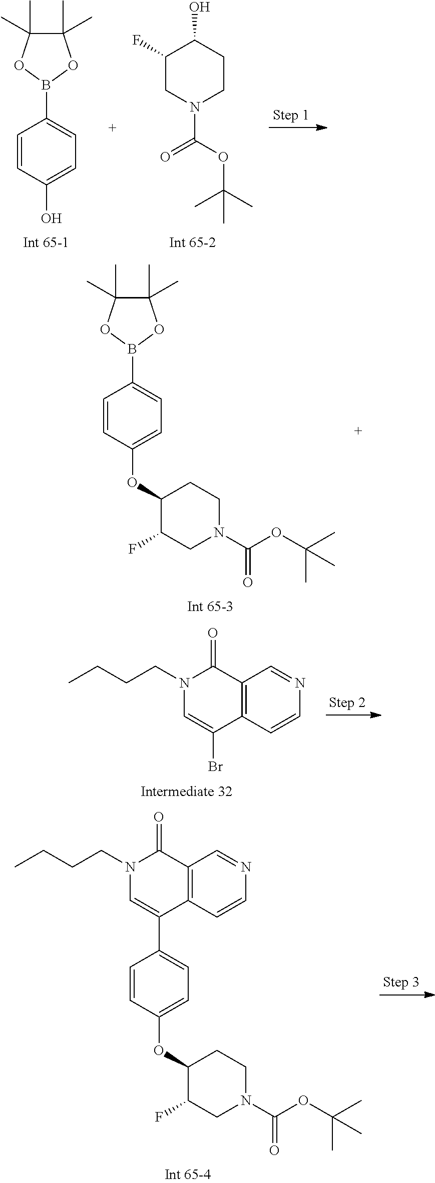

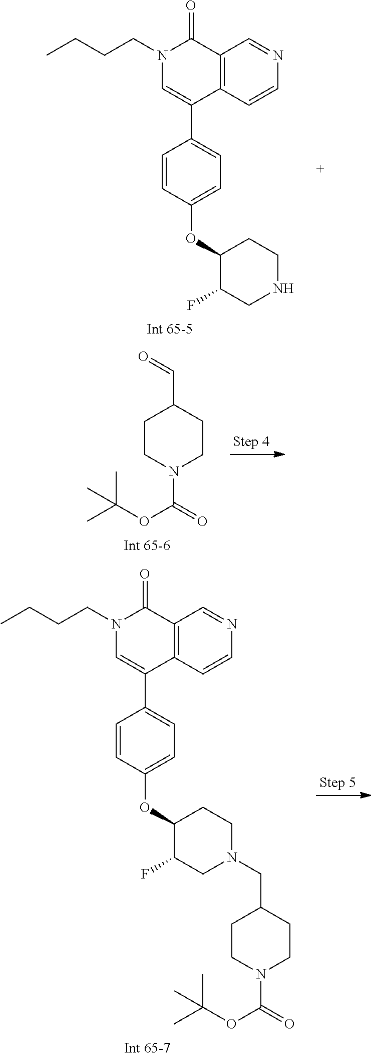 [SMILES: CCCCn1cc(-c2ccc(O[C@H]3CCNC[C@@H]3F)cc2)c2ccncc2c1=O ; CC(C)(C)OC(=O)N1CCC(C=O)CC1 ; CCCCn1cc(-c2ccc(O[C@H]3CCN(CC4CCN(C(=O)OC(C)(C)C)CC4)C[C@@H]3F)cc2)c2ccncc2c1=O]

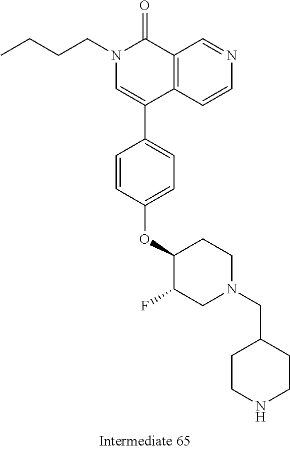 [SMILES: CCCCn1cc(-c2ccc(O[C@H]3CCN(CC4CCNCC4)C[C@@H]3F)cc2)c2ccncc2c1=O]

### Step 1: tert-butyl (3S,4S)-3-fluoro-4-(4-(4,4,5,5-tetramethyl-1,3,2-dioxaborolan-2-yl)phenoxy)piperidine-1-carboxylate (Int 65-3)

To a solution of 4-(4,4,5,5-tetramethyl-1,3,2-dioxaborolan-2-yl)phenol (Int 65-1, 160 mg, 0.727 mmol), tert-butyl (4R,3S)-3-fluoro-4-hydroxypiperidine-1-carboxylate (Int 65-2, 160 mg, 0.730 mmol) and PPh₃ (229 mg, 0.872 mmol) in THE (5 ml) under an argon atmosphere was dropwise added DIAD (0.170 ml, 0.872 mmol) at RT and the RM was stirred at RT for 20 h. The mixture was added to a mixture of EtOAc and an aq. sat. solution of NaHCO₃, the organic phase was washed with brine, dried over MgSO₄ and concentrated. The residue was purified by chromatography on silica gel eluting with EtOAc (from 0% to 20%) in CHX, yielding the title compound tert-butyl (3S,4S)-3-fluoro-4-(4-(4,4,5,5-tetramethyl-1,3,2-dioxaborolan-2-yl)phenoxy)piperidine-1-carboxylate, Int 65-3, as a solid (102 mg).

Method LCMS_MLG2: Rt=1.56 min; [M-Boc+H]⁺=322.

### Step 2: tert-butyl (3S,4S)-4-(4-(2-butyl-1-oxo-1,2-dihydro-2,7-naphthyridin-4-yl)phenoxy)-3-fluoropiperidine-1-carboxylate (Int 65-4)

To a mixture of 4-bromo-2-butyl-2,7-naphthyridin-1(2H)-one (Intermediate 32, 100 mg, 0.341 mmol), tert-butyl (3S,4S)-3-fluoro-4-(4-(4,4,5,5-tetramethyl-1,3,2-dioxaborolan-2-yl)phenoxy)piperidine-1-carboxylate (Int 65-3, 144 mg, 0.332 mmol) and Na₂CO₃ (105 mg, 0.995 mmol) in a mixture of 1,4-dioxane (4 ml) and water (1 ml) under an argon atmosphere was added PdCl₂(dppf)-CH₂Cl₂ (24 mg, 0.032 mmol) and the RM was heated at 100° C. for 2 h. The mixture was filtered through CELITE®, the solids were washed with EtOAc and the filtrate was concentrated. The residue was purified by reverse phase chromatography on a REDISEP® C18 column (15.5 g) eluting with ACN (from 0% to 100%) in an aq. solution of TFA (0.1%), yielding the title compound tert-butyl (3S,4S)-4-(4-(2-butyl-1-oxo-1,2-dihydro-2,7-naphthyridin-4-yl)phenoxy)-3-fluoropiperidine-1-carboxylate, Int 65-4, as a solid (164 mg).

Method LCMS_MLG2: Rt=1.33 min; [M+H]⁺=496.

### Step 3: 2-butyl-4-(4-(((3S,4S)-3-fluoropiperidin-4-yl)oxy)phenyl)-2,7-naphthyridin-1(2H)-one (Int 65-5)

To a solution of tert-butyl (3S,4S)-4-(4-(2-butyl-1-oxo-1,2-dihydro-2,7-naphthyridin-4-yl)phenoxy)-3-fluoropiperidine-1-carboxylate (Int 65-4, 178 mg, 0.330 mmol) in DCM (3 ml) was added TFA (0.750 ml, 9.73 mmol) and the RM was stirred at RT for 1 h. The solvents were removed and the residue was purified by reverse phase chromatography on a REDISEP® C18 column (15.5 g) eluting with ACN (from 0% to 100%) in an aq. solution of TFA (0.1%), yielding the title compound 2-butyl-4-(4-(((3S,4S)-3-fluoropiperidin-4-yl)oxy)phenyl)-2,7-naphthyridin-1(2H)-one, Int 65-5, as a solid TFA salt (169 mg).

Method LCMS_MLG2: Rt=0.54 min; [M+H]⁺=396.

### Step 4: tert-butyl 4-(((3S,4S)-4-(4-(2-butyl-1-oxo-1,2-dihydro-2,7-naphthyridin-4-yl)phenoxy)-3-fluoropiperidin-1-yl)methyl)piperidine-1-carboxylate (Int 65-7)

To a solution of 2-butyl-4-(4-(((3S,4S)-3-fluoropiperidin-4-yl)oxy)phenyl)-2,7-naphthyridin-1(2H)-one TFA salt (Int 65-5, 169 mg, 0.332 mmol), TEA (0.150 ml, 1.076 mmol) and tert-butyl 4-formylpiperidine-1-carboxylate (Int 65-6, 85 mg, 0.398 mmol) in MeOH (3 ml) under an argon atmosphere was added a solution of ZnCl₂ (0.7 M) in THE (0.6 ml, 0.420 mmol) and the RM was stirred at RT for 7 h. Solid NaBH₃CN (40 mg, 0.637 mmol) was added and the RM was stirred at RT overnight. The mixture was concentrated, yielding the title compound tert-butyl 4-(((3S,4S)-4-(4-(2-butyl-1-oxo-1,2-dihydro-2,7-naphthyridin-4-yl)phenoxy)-3-fluoropiperidin-1-yl)methyl)piperidine-1-carboxylate, Int 65-7, as a solid (197 mg), which was used for the next step without further purification.

Method LCMS_MLG2: Rt=1.09 min; [M+H]⁺=593.

### Step 5: 2-butyl-4-(4-(((3S,4S)-3-fluoro-1-(piperidin-4-ylmethyl)piperidin-4-yl)oxy)phenyl)-2,7-naphthyridin-1(2H)-one (Intermediate 65)

To a solution of tert-butyl 4-(((3S,4S)-4-(4-(2-butyl-1-oxo-1,2-dihydro-2,7-naphthyridin-4-yl)phenoxy)-3-fluoropiperidin-1-yl)methyl)piperidine-1-carboxylate (Int 65-7, 197 mg, 0.332 mmol) in DCM (3 ml) was added TFA (0.750 ml, 9.73 mmol) and the RM was stirred at RT for 1 h. The mixture was concentrated and the residue was purified by reverse phase chromatography on a REDISEP® C18 column (15.5 g) eluting with ACN (from 0% to 100%) in an aq. solution of TFA (0.1%), yielding the title compound 2-butyl-4-(4-(((3S,4S)-3-fluoro-1-(piperidin-4-ylmethyl)piperidin-4-yl)oxy)phenyl)-2,7-naphthyridin-1(2H)-one, Intermediate 65, as a solid TFA salt (245 mg).

Method LCMS_MLG2: Rt=0.52 min; [M+H]⁺=493.

### Intermediate 66: 2-butyl-4-(4-(((3S,4S)-3-fluoro-1-(piperidin-4-ylmethyl)piperidin-4-yl)oxy)-3-methoxyphenyl)-2,7-naphthyridin-1(2H)-one

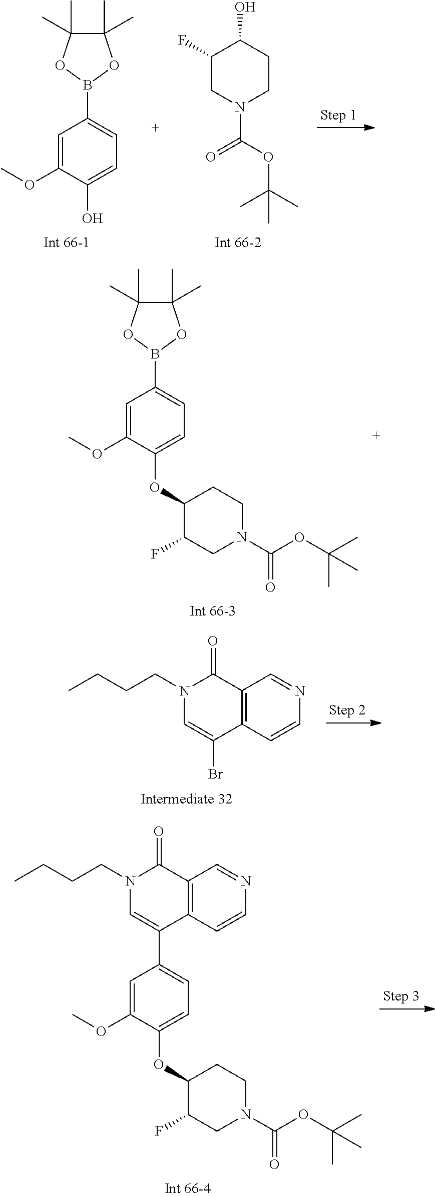

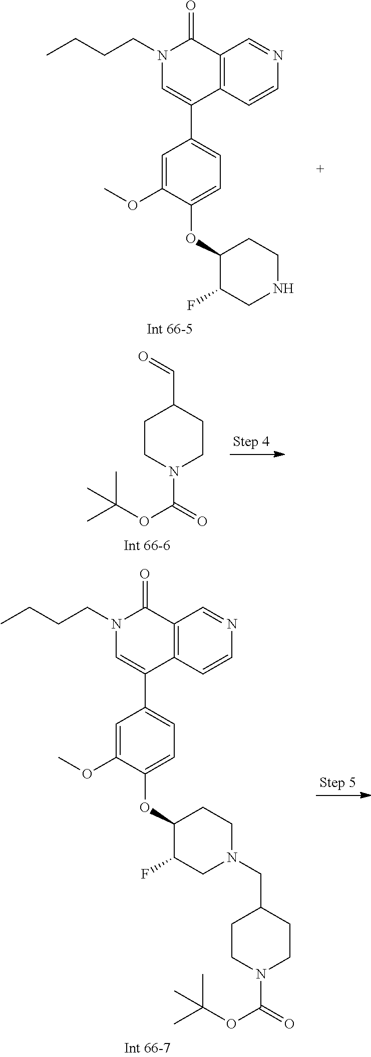 [SMILES: CCCCn1cc(-c2ccc(O[C@H]3CCNC[C@@H]3F)c(OC)c2)c2ccncc2c1=O ; CC(C)(C)OC(=O)N1CCC(C=O)CC1 ; CCCCn1cc(-c2ccc(O[C@H]3CCN(CC4CCN(C(=O)OC(C)(C)C)CC4)C[C@@H]3F)c(OC)c2)c2ccncc2c1=O]

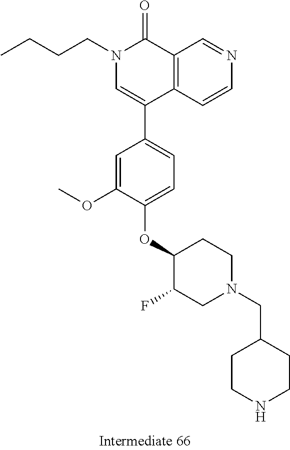 [SMILES: CCCCn1cc(-c2ccc(O[C@H]3CCN(CC4CCNCC4)C[C@@H]3F)c(OC)c2)c2ccncc2c1=O]

### Step 1: tert-butyl (3S,4S)-3-fluoro-4-(2-methoxy-4-(4,4,5,5-tetramethyl-1,3,2-dioxaborolan-2-yl)phenoxy)piperidine-1-carboxylate (Int 66-3)

To a solution of 4-hydroxy-3-methoxyphenylboronic acid pinacol ester (Int 66-1, 321 mg, 1.283 mmol), tert-butyl (4R,3S)-3-fluoro-4-hydroxypiperidine-1-carboxylate (Int 66-2, 300 mg, 1.368 mmol) and PPh₃ (404 mg, 1.540 mmol) in THE (4 ml) was dropwise added DIAD (0.299 ml, 1.540 mmol) under an argon atmosphere and the RM was stirred at RT for 24 h. The mixture was added to a mixture of EtOAc and a sat. solution of NaHCO₃, the organic phase was washed with brine, dried over MgSO₄ and concentrated. The residue was purified by chromatography on silica gel eluting with EtOAc (from 0% to 30%) in CHX, yielding the title compound tert-butyl (3S,4S)-3-fluoro-4-(2-methoxy-4-(4,4,5,5-tetramethyl-1,3,2-dioxaborolan-2-yl)phenoxy)piperidine-1-carboxylate, Int 66-3, as a solid (397 mg).

Method LCMS_MLG8: Rt=1.49 min; [M-Boc+H]⁺=352.

### Step 2: tert-butyl (3S,4S)-4-(4-(2-butyl-1-oxo-1,2-dihydro-2,7-naphthyridin-4-yl)-2-methoxyphenoxy)-3-fluoropiperidine-1-carboxylate (Int 66-4)

To a mixture of 4-bromo-2-butyl-2,7-naphthyridin-1(2H)-one (Intermediate 32, 152 mg, 0.542 mmol), tert-butyl (3S,4S)-3-fluoro-4-(2-methoxy-4-(4,4,5,5-tetramethyl-1,3,2-dioxaborolan-2-yl)phenoxy)piperidine-1-carboxylate (Int 66-3, 334 mg, 0.451 mmol) and Na₂CO₃ (144 mg, 1.354 mmol) in a mixture of 1,4-dioxane (4 ml) and water (1 ml) under an argon atmosphere was added PdCl₂(dppf)-CH₂Cl₂ (33 mg, 0.045 mmol). The RM was heated at 100° C. for 2 h. The mixture was filtered through CELITE®, the solids were washed with EtOAc, the filtrate was concentrated and the residue was purified by reverse phase chromatography on a REDISEP® Gold HP C18 column (15.5 g) eluting with ACN (from 1% to 100%) in an aq. solution of TFA (0.1%), yielding the title compound tert-butyl (3S,4S)-4-(4-(2-butyl-1-oxo-1,2-dihydro-2,7-naphthyridin-4-yl)-2-methoxyphenoxy)-3-fluoropiperidine-1-carboxylate, Int 66-4, as a solid (284 mg).

Method LCMS_MLG8: Rt=1.29 min; [M-Boc+H]⁺=526.

### Step 3: 2-butyl-4-(4-(((3S,4S)-3-fluoropiperidin-4-yl)oxy)-3-methoxyphenyl)-2,7-naphthyridin-1(2H)-one (Int 66-5)

To a solution of tert-butyl (3S,4S)-4-(4-(2-butyl-1-oxo-1,2-dihydro-2,7-naphthyridin-4-yl)-2-methoxyphenoxy)-3-fluoropiperidine-1-carboxylate (Int 66-4, 280 mg, 0.511 mmol) in DCM (5 ml) was added TFA (1 ml, 12.98 mmol) and the RM was stirred at RT for 1 h. The mixture was concentrated and the residue was purified by reverse phase chromatography on a REDISEP® Gold HP C18 column (15.5 g) eluting with ACN (from 1% to 100%) in an aq. solution of TFA (0.1%), yielding the title compound 2-butyl-4-(4-(((3S,4S)-3-fluoropiperidin-4-yl)oxy)-3-methoxyphenyl)-2,7-naphthyridin-1(2H)-one, Int 66-5, as a solid TFA salt (285 mg).

Method LCMS_MLG8: Rt=0.54 min; [M+H]⁺=426.

### Step 4: tert-butyl 4-(((3S,4S)-4-(4-(2-butyl-1-oxo-1,2-dihydro-2,7-naphthyridin-4-yl)-2-methoxyphenoxy)-3-fluoropiperidin-1-yl)methyl)piperidine-1-carboxylate (Int 66-7)

To a solution of 2-butyl-4-(4-(((3S,4S)-3-fluoropiperidin-4-yl)oxy)-3-methoxyphenyl)-2,7-naphthyridin-1(2H)-one TFA salt (Int 66-5, 285 mg, 0.528 mmol), TEA (0.250 ml, 1.794 mmol) and tert-butyl 4-formylpiperidine-1-carboxylate (Int 66-6, 135 mg, 0.634 mmol) in MeOH (5 ml) was added a solution of ZnCl₂ (0.5 M) in TH (1.2 ml, 0.600 mmol) and the RM was stirred under an argon atmosphere at RT for 7 h. Solid NaBH₃CN (37 mg, 0.589 mmol) was added and the RM was stirred at RT for 24 h. The mixture was concentrated, yielding the title compound tert-butyl 4-(((3S,4S)-4-(4-(2-butyl-1-oxo-1,2-dihydro-2,7-naphthyridin-4-yl)-2-methoxyphenoxy)-3-fluoropiperidin-1-yl)methyl)piperidine-1-carboxylate, Int 66-7, as a solid (329 mg), which was used for the next step without further purification.

Method LCMS_MLG8: Rt=0.99 min; [M+H]⁺=623.

### Step 5: 2-butyl-4-(4-(((3S,4S)-3-fluoro-1-(piperidin-4-ylmethyl)piperidin-4-yl)oxy)-3-methoxyphenyl)-2,7-naphthyridin-1(2H)-one (Intermediate 66)

To a solution of tert-butyl 4-(((3S,4S)-4-(4-(2-butyl-1-oxo-1,2-dihydro-2,7-naphthyridin-4-yl)-2-methoxyphenoxy)-3-fluoropiperidin-1-yl)methyl)piperidine-1-carboxylate (Int 66-7, 0.53 mmol) in DCM (5 ml) was added TFA (1 ml, 12.98 mmol) and the RM stirred at RT for 1 h. The mixture was concentrated and the residue was purified by reverse phase chromatography on a REDISEP® Gold HP C18 column (15.5 g) eluting with ACN (from 1% to 100%) in an aq. solution of TFA (0.1%), yielding the title compound 2-butyl-4-(4-(((3S,4S)-3-fluoro-1-(piperidin-4-ylmethyl)piperidin-4-yl)oxy)-3-methoxyphenyl)-2,7-naphthyridin-1(2H)-one, Intermediate 66, as a solid TFA salt (305 mg).

Method LCMS_MLG8: Rt=0.47 min; [M+H]⁺=523.

### Intermediate 67: 2-butyl-4-(4-(((3S,4S)-3-fluoro-1-(piperidin-4-ylmethyl)piperidin-4-yl)oxy)-3-methoxyphenyl)-2,7-naphthyridin-1(2H)-one

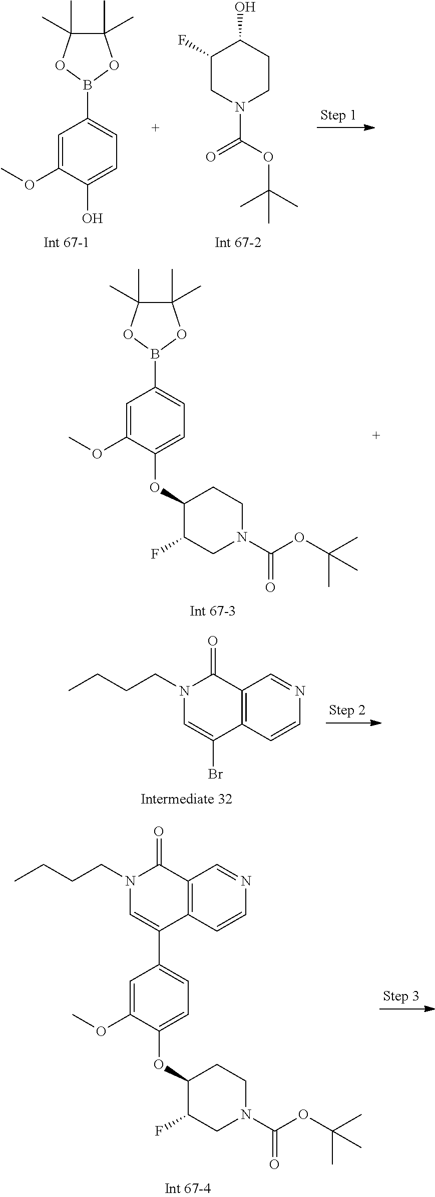

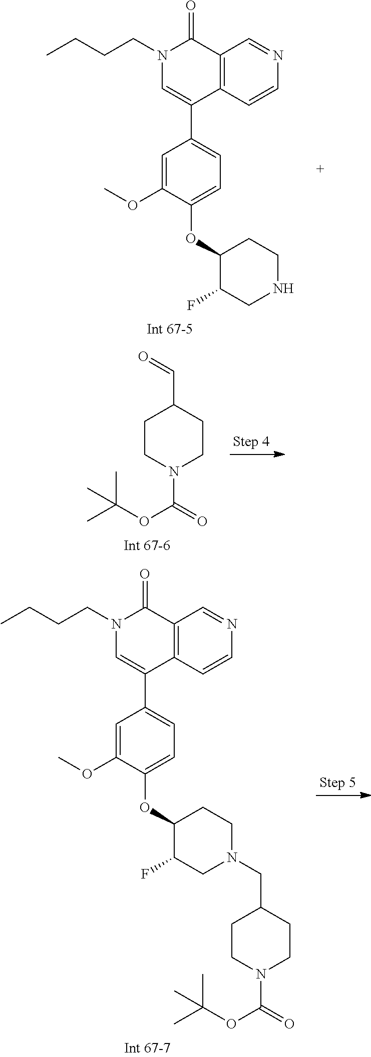 [SMILES: CCCCn1cc(-c2ccc(O[C@H]3CCNC[C@@H]3F)c(OC)c2)c2ccncc2c1=O ; CC(C)(C)OC(=O)N1CCC(C=O)CC1 ; CCCCn1cc(-c2ccc(O[C@H]3CCN(CC4CCN(C(=O)OC(C)(C)C)CC4)C[C@@H]3F)c(OC)c2)c2ccncc2c1=O]

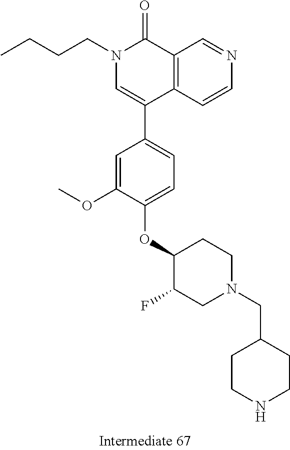 [SMILES: CCCCn1cc(-c2ccc(O[C@H]3CCN(CC4CCNCC4)C[C@@H]3F)c(OC)c2)c2ccncc2c1=O]

### Step 1: tert-butyl (3S,4S)-3-fluoro-4-(2-methoxy-4-(4,4,5,5-tetramethyl-1,3,2-dioxaborolan-2-yl)phenoxy)piperidine-1-carboxylate (Int 67-3)

To a solution of 4-hydroxy-3-methoxyphenylboronic acid pinacol ester (Int 67-1, 963 mg, 3.85 mmol), tert-butyl (4R,3S)-3-fluoro-4-hydroxypiperidine-1-carboxylate (Int 67-2, 900 mg, 4.10 mmol) and PPh₃ (1212 mg, 4.62 mmol) in THE (24 ml) under an argon atmosphere was added DIAD (0.900 ml, 4.63 mmol) and the RM was stirred at RT for 2 days. The mixture was added into a mixture of EtOAc and an aq. sat. solution of NaHCO₃, the organic phase was washed with brine, dried over MgSO₄ and concentrated. The residue was purified by chromatography on silica gel eluting with EtOAc (from 0% to 20%) in CHX, yielding the title compound tert-butyl (3S,4S)-3-fluoro-4-(2-methoxy-4-(4,4,5,5-tetramethyl-1,3,2-dioxaborolan-2-yl)phenoxy)piperidine-1-carboxylate, Int 67-3, as a solid (1.02 g).

Method LCMS_MLG2: Rt=1.47 min; [M-Boc+H]⁺=352.

### Step 2: tert-butyl (3S,4S)-4-(4-(2-butyl-1-oxo-1,2-dihydro-2,7-naphthyridin-4-yl)-2-methoxyphenoxy)-3-fluoropiperidine-1-carboxylate (Int 67-4)

To a mixture of 4-bromo-2-butyl-2,7-naphthyridin-1(2H)-one (Intermediate 32, 172 mg, 0.612 mmol), tert-butyl (3S,4S)-3-fluoro-4-(2-methoxy-4-(4,4,5,5-tetramethyl-1,3,2-dioxaborolan-2-yl)phenoxy)piperidine-1-carboxylate (Int 67-3, 325 mg, 0.612 mmol) and Na₂CO₃ (195 mg, 1.836 mmol) in a mixture of 1,4-dioxane (4 ml) and water (1 ml) under an argon atmosphere was added PdCl₂(dppf)-CH₂Cl₂ (45 mg, 0.061 mmol) and the RM was heated at 100° C. for 2 h. The mixture was filtered through CELITE®, the solids were washed with EtOAc and the filtrate was concentrated, yielding the title compound tert-butyl (3S,4S)-4-(4-(2-butyl-1-oxo-1,2-dihydro-2,7-naphthyridin-4-yl)-2-methoxyphenoxy)-3-fluoropiperidine-1-carboxylate, Int 67-4, as a solid (322 mg), which was used for the next step without further purification.

Method LCMS_MLG5: Rt=1.12 min; [M+H]⁺=526.

### Step 3: 2-butyl-4-(4-(((3S,4S)-3-fluoropiperidin-4-yl)oxy)-3-methoxyphenyl)-2,7-naphthyridin-1(2H)-one (Int 67-5)

To a solution of tert-butyl (3S,4S)-4-(4-(2-butyl-1-oxo-1,2-dihydro-2,7-naphthyridin-4-yl)-2-methoxyphenoxy)-3-fluoropiperidine-1-carboxylate (Int 67-4, 280 mg, 0.511 mmol) in DCM (5 ml) was added TFA (1 ml, 12.98 mmol) and the RM was stirred at RT for 1 h. The mixture was concentrated and the residue was purified by reverse phase chromatography on a REDISEP® C18 column (15.5 g) eluting with ACN (2% to 100%) in an aq. solution of TFA (0.1%), yielding the title compound 2-butyl-4-(4-(((3S,4S)-3-fluoropiperidin-4-yl)oxy)-3-methoxyphenyl)-2,7-naphthyridin-1(2H)-one, Int 67-5, as a solid TFA salt (339 mg).

Method LCMS_IJ1: Rt=0.55 min; [M+H]⁺=426.

### Step 4: tert-butyl 4-(((3S,4S)-4-(4-(2-butyl-1-oxo-1,2-dihydro-2,7-naphthyridin-4-yl)-2-methoxyphenoxy)-3-fluoropiperidin-1-yl)methyl)piperidine-1-carboxylate (Int 67-7)

To a solution of 2-butyl-4-(4-(((3S,4S)-3-fluoropiperidin-4-yl)oxy)-3-methoxyphenyl)-2,7-naphthyridin-1(2H)-one TFA salt (Int 67-5, 339 mg, 0.503 mmol), TEA (0.210 ml, 1.508 mmol), tert-butyl 4-formylpiperidine-1-carboxylate (Int 67-6, 129 mg, 0.603 mmol) in MeOH (5 ml) under an argon atmosphere was added a solution of ZnCl₂ (0.7 M) in THE (0.9 ml, 0.630 mmol) at RT and the RM was stirred at RT for 7 h. Solid NaBH₃CN (47 mg, 0.748 mmol) was added and the RM was stirred overnight at RT. The mixture was concentrated, yielding the title compound tert-butyl 4-(((3S,4S)-4-(4-(2-butyl-1-oxo-1,2-dihydro-2,7-naphthyridin-4-yl)-2-methoxyphenoxy)-3-fluoropiperidin-1-yl)methyl)piperidine-1-carboxylate, Int 67-7, as a solid (313 mg), which was used for the next step without further purification.

Method LCMS_IJ1: Rt=0.97 min; [M+H]⁺=623.

### Step 5: 2-butyl-4-(4-(((3S,4S)-3-fluoro-1-(piperidin-4-ylmethyl)piperidin-4-yl)oxy)-3-methoxyphenyl)-2,7-naphthyridin-1(2H)-one (Intermediate 67)

To a solution of tert-butyl 4-(((3S,4S)-4-(4-(2-butyl-1-oxo-1,2-dihydro-2,7-naphthyridin-4-yl)-2-methoxyphenoxy)-3-fluoropiperidin-1-yl)methyl)piperidine-1-carboxylate (Int 67-7, 313 mg, 0.503 mmol) in DCM (5 ml) was added TFA (1 ml, 12.98 mmol) and the RM was stirred at RT for 1 h. The mixture was concentrated and the residue was purified by reverse phase chromatography on a REDISEP® C18 column (15.5 g) eluting with ACN (from 2% to 100%) in an aq. solution of TFA (0.1%), yielding the title compound 2-butyl-4-(4-(((3S,4S)-3-fluoro-1-(piperidin-4-ylmethyl)piperidin-4-yl)oxy)-3-methoxyphenyl)-2,7-naphthyridin-1(2H)-one, Intermediate 67, as a solid TFA salt (381 mg).

Method LCMS_IJ1: Rt=0.55 min; [M+H]⁺=523.

### Intermediate 68: 2-butyl-4-(3-fluoro-4-(((3R,4R)-3-fluoro-1-(piperidin-4-ylmethyl)piperidin-4-yl)oxy)phenyl)-2,7-naphthyridin-1(2H)-one

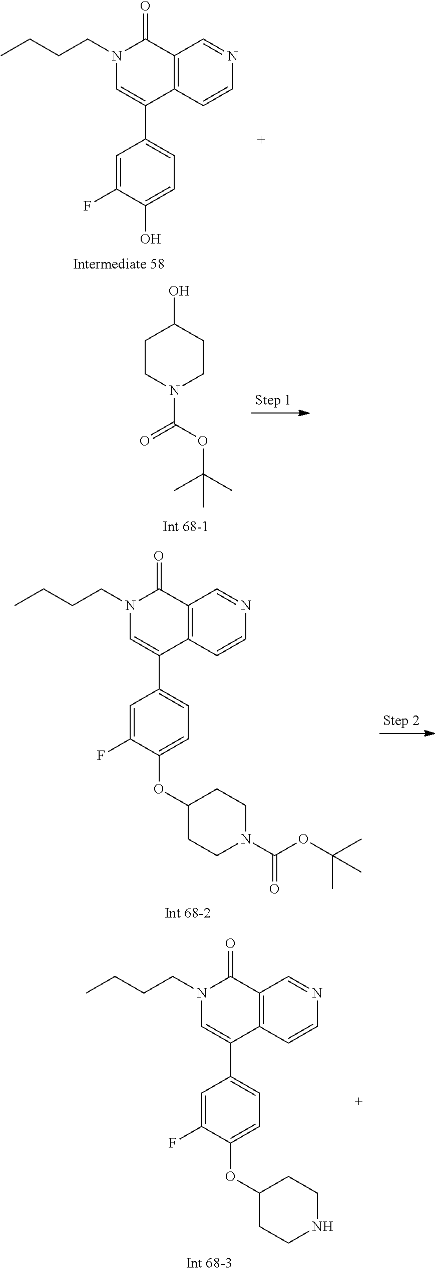

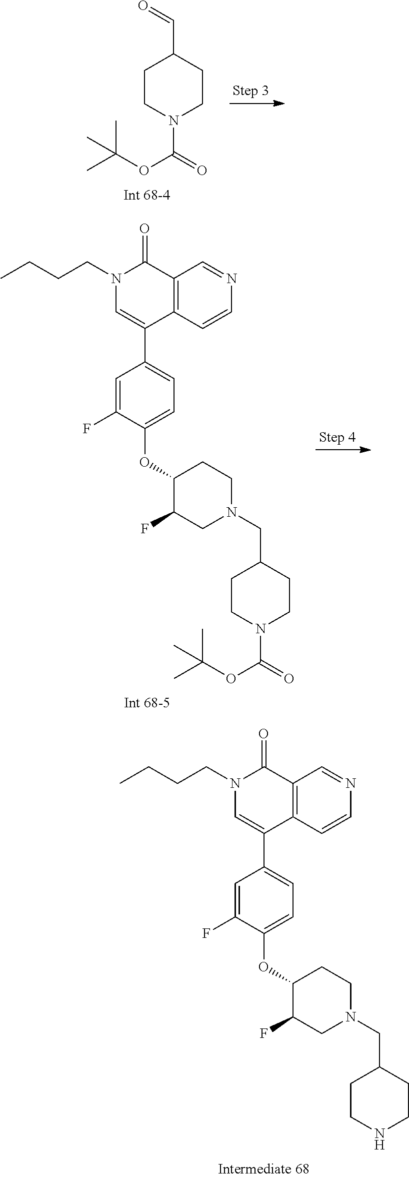 [SMILES: CC(C)(C)OC(=O)N1CCC(C=O)CC1 ; CCCCn1cc(-c2ccc(O[C@@H]3CCN(CC4CCN(C(=O)OC(C)(C)C)CC4)C[C@H]3F)c(F)c2)c2ccncc2c1=O ; CCCCn1cc(-c2ccc(O[C@@H]3CCN(CC4CCNCC4)C[C@H]3F)c(F)c2)c2ccncc2c1=O]

### Step 1: tert-butyl 4-(4-(2-butyl-1-oxo-1,2-dihydro-2,7-naphthyridin-4-yl)-2-fluorophenoxy)piperidine-1-carboxylate (Int 68-2)

To a solution of 2-butyl-4-(3-fluoro-4-hydroxyphenyl)-2,7-naphthyridin-1(2H)-one (Intermediate 58, 150 mg, 0.480 mmol), tert-butyl 4-hydroxypiperidine-1l-carboxylate (Int 68-1, 108 mg, 0.526 mmol) and PPh₃ (139 mg, 0.530 mmol) in THE (4 ml) under an argon atmosphere was dropwise added DIAD (0.110 ml, 0.566 mmol) at RT and the RMV was stirred at RT for 20 h. The mixture was concentrated and the residue was dissolved in toluene (4 ml). Tert-butyl 4-hydroxypiperidine-1-carboxylate (Int 68-1, 108 mg, 0.526 mmol) and PPh₃ (139 mg, 0.530 mmol) were added under an argon atmosphere, followed by DEAD (0.090 ml, 0.568 mmol) and the RM was stirred at RT for 20 h. The mixture was added into a mixture of EtOAc and a sat. solution of NaHCO₃, the organic phase was washed with brine, dried over MgSO₄ and concentrated. The residue was purified by chromatography on silica gel eluting with EtOAc (from 0% to 100%) in CHX, yielding the title compound tert-butyl 4-(4-(2-butyl-1-oxo-1,2-dihydro-2,7-naphthyridin-4-yl)-2-fluorophenoxy)piperidine-1-carboxylate, Int 68-2, as a solid (659 mg).

Method LCMS_MLG8: Rt=1.32 min; [M+H]⁺=496.

### Step 2: 2-butyl-4-(3-fluoro-4-(piperidin-4-yloxy)phenyl)-2,7-naphthyridin-1(2H)-one (Int 68-3)

To a solution of tert-butyl 4-(4-(2-butyl-1-oxo-1,2-dihydro-2,7-naphthyridin-4-yl)-2-fluorophenoxy)piperidine-1-carboxylate (Int 68-2, 0.093 mmol) in DCM (2 ml) was added TFA (0.200 ml, 2.600 mmol) and the RM was stirred at RT for 1 h. The mixture was concentrated and the residue was purified by reverse phase chromatography on a REDISEP® Gold HP C18 column (15.5 g) eluting with ACN (from 1% to 100%) in an aq. solution of TFA (0.1%), yielding the title compound 2-butyl-4-(3-fluoro-4-(piperidin-4-yloxy)phenyl)-2,7-naphthyridin-1(2H)-one, Int 68-3, as a solid TFA salt (28 mg).

Method LCMS_MLG8: Rt=0.61 min; [M+H]⁺=396.

### Step 3: tert-butyl 4-(((3R,4R)-4-(4-(2-butyl-1-oxo-1,2-dihydro-2,7-naphthyridin-4-yl)-2-fluorophenoxy)-3-fluoropiperidin-1-yl)methyl)piperidine-1-carboxylate (Int 68-5)

To a solution of 2-butyl-4-(3-fluoro-4-(piperidin-4-yloxy)phenyl)-2,7-naphthyridin-1(2H)-one TFA salt (Int 68-3, 170 mg, 0.322 mmol), TEA (0.150 ml, 1.076 mmol) and tert-butyl 4-formylpiperidine-1-carboxylate (Int 68-4, 89 mg, 0.419 mmol) in MeOH (4 ml) at RT was added a solution of ZnCl₂ (0.7 M) in THF (0.500 ml, 0.350 mmol) and the RM was stirred under an argon atmosphere at RT for 7 h. Solid NaBH₃CN (35 mg, 0.557 mmol) was added and the RM was stirred at RT for 18 h. The mixture was concentrated, yielding the title compound tert-butyl 4-(((3R,4R)-4-(4-(2-butyl-1-oxo-1,2-dihydro-2,7-naphthyridin-4-yl)-2-fluorophenoxy)-3-fluoropiperidin-1-yl)methyl)piperidine-1-carboxylate, Int 68-5, as a solid (249 mg), which was used for the next step without further purification.

Method LCMS_MLG9: Rt=0.78 min; [M+H]⁺=611.

### Step 4: 2-butyl-4-(3-fluoro-4-(((3R,4R)-3-fluoro-1-(piperidin-4-ylmethyl)piperidin-4-yl)oxy)phenyl)-2,7-naphthyridin-1(2H)-one (Intermediate 68)

To a solution of tert-butyl 4-(((3R,4R)-4-(4-(2-butyl-1-oxo-1,2-dihydro-2,7-naphthyridin-4-yl)-2-fluorophenoxy)-3-fluoropiperidin-1-yl)methyl)piperidine-1-carboxylate (Int 68-5, 0.303 mmol) in DCM (3 ml) was added TFA (0.700 ml, 9.09 mmol) and the RM was stirred at RT for 1 h. The mixture was concentrated and purified by reverse phase chromatography on a REDISEP® Gold HP C18 column (15.5 g) eluting with ACN (from 1% to 100%) in an aq. solution of TFA (0.1%), yielding the title compound 2-butyl-4-(3-fluoro-4-(((3R,4R)-3-fluoro-1-(piperidin-4-ylmethyl)piperidin-4-yl)oxy)phenyl)-2,7-naphthyridin-1(2H)-one, Intermediate 68, as a solid TFA salt (243 mg).

Method LCMS_MLG9: Rt=0.53 min; [M+H]⁺=511.

### Intermediate 69: 2-butyl-4-(4-(((cis)-4-(piperidin-4-yloxy)cyclohexyl)oxy)phenyl)-2,7-naphthyridin-1(2H)-one

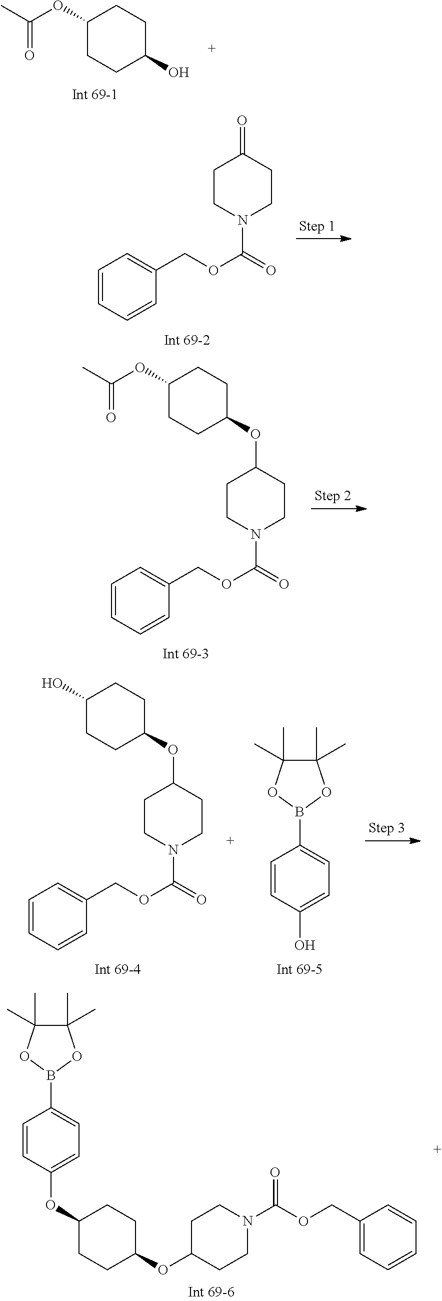

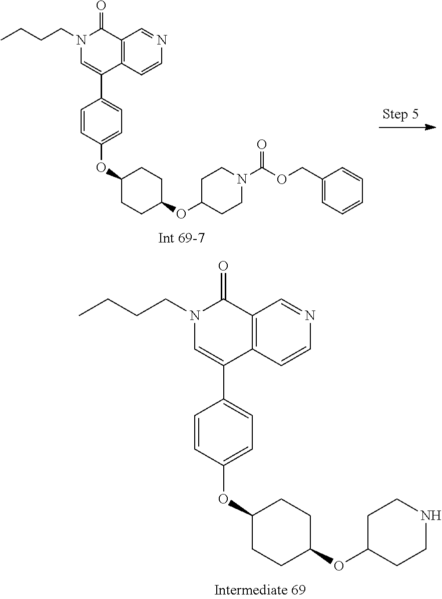 [SMILES: CCCCn1cc(-c2ccc(O[C@H]3CC[C@@H](OC4CCN(C(=O)OCc5ccccc5)CC4)CC3)cc2)c2ccncc2c1=O ; CCCCn1cc(-c2ccc(O[C@H]3CC[C@@H](OC4CCNCC4)CC3)cc2)c2ccncc2c1=O]

### Step 1: Benzyl 4-(((trans)-4-acetoxycyclohexyl)oxy)piperidine-1-carboxylate (Int 69-3)

To a solution of (trans)-4-hydroxycyclohexyl acetate (Int 69-1, 1.538 g, 9.24 mmol) in THF (25 ml) was added TEA (1.416 ml, 10.16 mmol) at 0° C., followed by the dropwise addition of trimethylsilyl chloride (1.239 ml, 9.70 mmol). The RM was stirred at 0° C. for 1 h and diluted in hexane. The mixture was filtered, the solids were washed with hexane, the filtrate was concentrated and the residue was dissolved in DCM (25 ml) and cooled to −60° C. To the stirred RM were added benzyl 4-oxopiperidine-1-carboxylate (Int 69-2, 2154 mg, 9.24 mmol), triethylsilane (1.623 ml, 10.16 mmol) and trimethylsilyl trifluoromethanesulfonate (0.834 ml, 4.62 mmol). The RM was allowed to warm to 0° C. and the RM was stirred at RT for 1 h. The mixture was diluted with EtOAc and an aq. solution of H₃PO₄ (1 M) was added. The organic phase was washed with brine, dried over MgSO₄ and concentrated. The residue was purified by chromatography on silica gel eluting with EtOAc (from 0% to 100%) in CHX, yielding the title compound benzyl 4-((trans-4-acetoxycyclohexyl)oxy)piperidine-1-carboxylate, Int 69-3, as a solid (2.270 g).

Method LCMS_MLG2: Rt=1.25 min; [M+H]⁺=376.

### Step 2: Benzyl 4-(((trans)-4-hydroxycyclohexyl)oxy)piperidine-1-carboxylate (Int 69-4)

To a solution of benzyl 4-(((trans)-4-acetoxycyclohexyl)oxy)piperidine-1-carboxylate (Int 69-3, 2.270 g, 5.74 mmol) in MeOH (55 ml) was added sodium methoxide (155 mg, 2.87 mmol) and the RM was stirred at RT for 20 h. The mixture was diluted with EtOAc and water, the aq. phase was extracted with EtOAc, the combined organic phases were washed with brine, dried over MgSO₄ and concentrated, yielding benzyl 4-(((trans)-4-hydroxycyclohexyl)oxy)piperidine-1-carboxylate, Int 69-4, as an oil (1.862 g), which was used for the next step without further purification.

Method LCMS_MLG2: Rt=0.94 min; [M+H]⁺=334.

### Step 3: Benzyl 4-(((cis)-4-(4-(4,4,5,5-tetramethyl-1,3,2-dioxaborolan-2-yl)phenoxy)cyclohexyl)oxy)piperidine-1-carboxylate (Int 69-6)

To a solution of benzyl 4-(((trans)-4-hydroxycyclohexyl)oxy)piperidine-1-carboxylate (Int 69-4, 86 mg, 0.219 mmol), 4-hydroxyphenyl boronic acid pinacol ester (Int 69-5, 59 mg, 0.263 mmol), PPh₃ (62 mg, 0.236 mmol) in THE (3 ml) under an argon atmosphere was dropwise added DIAD (0.051 ml, 0.263 mmol) and the RM was stirred at RT for 20 h. The mixture was purified by reverse phase chromatography on a REDISEP® Gold HP C18 column (15.5 g) eluting with ACN (from 1% to 100%) in an aq. solution of TFA (0.1%), yielding the title compound benzyl 4-(((cis)-4-(4-(4,4,5,5-tetramethyl-1,3,2-dioxaborolan-2-yl)phenoxy)cyclohexyl)oxy)piperidine-1-carboxylate, Int 69-6, as a solid (91 mg).

¹H NMR (400 MHz, DMSO-d₆) δ[ppm] 7.58 (d, J=8.3 Hz, 2H), 7.45-7.26 (m, 5H), 6.92 (d, J=8.4 Hz, 2H), 5.06 (s, 2H), 4.48 (m, 1H), 3.74-3.51 (m, 4H), 3.14 (m, 2H), 1.85-1.55 (m, 10H), 1.37 (m, 2H), 1.27 (s, 12H).

### Step 4: Benzyl 4-(((cis)-4-(4-(2-butyl-1-oxo-1,2-dihydro-2,7-naphthyridin-4-yl)phenoxy)cyclohexyl)oxy)piperidine-1-carboxylate (Int 69-7)

To a mixture of 4-(((cis)-4-(4-(4,4,5,5-tetramethyl-1,3,2-dioxaborolan-2-yl)phenoxy)cyclohexyl)oxy)piperidine-1-carboxylate (Int 69-6, 89 mg, 0.166 mmol), 4-bromo-2-butyl-2,7-naphthyridin-1(2H)-one (Intermediate 32, 45 mg, 0.160 mmol) and Na₂CO₃ (51 mg, 0.481 mmol) in a mixture of 1,4-dioxane (4 ml) and water (1 ml) under an argon atmosphere was added PdCl₂(dppf)-CH₂Cl₂(12 mg, 0.016 mmol). The RM was heated at 100° C. for 2 h. The mixture was filtered through CELITE® and the solids were washed with EtOAc. The filtrate was concentrated and the residue was purified by chromatography on silica gel eluting with EtOAc (from 0% to 100%) in CHX, yielding the title compound benzyl 4-(((cis)-4-(4-(2-butyl-1-oxo-1,2-dihydro-2,7-naphthyridin-4-yl)phenoxy)cyclohexyl)oxy)piperidine-1-carboxylate, Int 69-7, as a solid (72 mg).

Method LCMS_MLG2: Rt=1.57; [M+H]⁺=610.

### Step 5: 2-butyl-4-(4-(((cis)-4-(piperidin-4-yloxy)cyclohexyl)oxy)phenyl)-2,7-naphthyridin-1(2H)-one (Intermediate 69)

To a solution of benzyl 4-(((cis)-4-(4-(2-butyl-1-oxo-1,2-dihydro-2,7-naphthyridin-4-yl)phenoxy)cyclohexyl)oxy)piperidine-1-carboxylate (Int 69-7, 72 mg, 0.118 mmol) in MeOH (1.5 ml) at RT under an argon atmosphere was added and Pd/C (10%, 10 mg, 0.009 mmol). The argon atmosphere was replaced by hydrogen and the RM was stirred at RT for 20 h. The mixture was filtered through CELITE®, the solids were washed with MeOH and the filtrate was concentrated, yielding the title compound benzyl 4-(((cis)-4-(4-(2-butyl-1-oxo-1,2-dihydro-2,7-naphthyridin-4-yl)phenoxy)cyclohexyl)oxy)piperidine-1-carboxylate, Intermediate 69, as a solid (73 mg).

Method LCMS_MLG2: Rt=0.81; [M+H]⁺=476.

### Intermediate 72:3-(2,4-dioxotetrahydropyrimidin-1(2H)-yl)-4-fluorobenzoic Acid

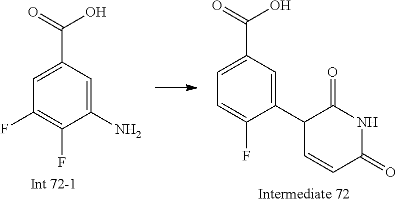 [SMILES: Nc1cc(C(=O)O)cc(F)c1F ; O=C1C=CC(c2cc(C(=O)O)ccc2F)C(=O)N1]

To a solution of 3-amino-4-fluorobenzoic acid (Int 72-1, 9.3 g, 60 mmol) in HOAc (40 ml) at 25° C. were added acrylic acid (13 g, 180 mmol) and 18 drops of conc. H2SO₄ and the RM was stirred at 100° C. for 5.5 h. HOAc (60 ml) and urea (18.0 g, 300.0 mmol) were added and the RM was stirred at 120° C. for 26 h. The mixture was concentrated, ice and water were added, followed by a conc. aq. solution of HCl (37%). The mixture was filtered, the filtrate was saturated with solid NaCl and cooled to 15° C. for 20 h. The solid was collected and dried, yielding a first crop (5 g). The filtrate was concentrated, the residue was diluted with water and the resulting mixture was cooled to 15° C. for 20 h. The solid was filtered and dried yielding a second crop (4 g). To the combined crops (9 g) were added HOAc and the mixture was sonicated for 10 min. MTBE was added, the upper phase was decanted and this process was repeated one more time. The residue was concentrated, HOAc and MTBE were added and the mixture was sonicated. Additional MTBE was added, the upper phase was decanted and this process was repeated one more time. MTBE was added, the mixture was cooled to 15° C. for 20 h and the MTBE phase was decanted. The mixture was concentrated, an aq. solution of HCl (0.001 M) was added, the mixture was filtered and the solids were washed with an aq. solution of HCl (0.001 M), ACN and dried, yielding the title compound 3-(2,4-dioxotetrahydropyrimidin-1(2H)-yl)-4-fluorobenzoic acid, Intermediate 72, as a solid (4.0 g).

Method LCMS_A031: Rt=1.64 min; [M+H]⁺=253.

¹H NMR (500 MHz, DMSO-d₆) δ[ppm] 13.16 (brs, 1H), 10.54 (s, 1H), 8.02 (dd, J=7.4, 2.0 Hz, 1H), 7.93 (m, 1H), 7.44 (dd, J=9.8, 8.9 Hz, 1H), 3.77 (t, J=6.6 Hz, 2H), 2.74 (t, J=6.6 Hz, 2H).

### Intermediate 73: 2-(4-(2,4-dioxotetrahydropyrimidin-1(2H)-yl)phenoxy)acetic Acid

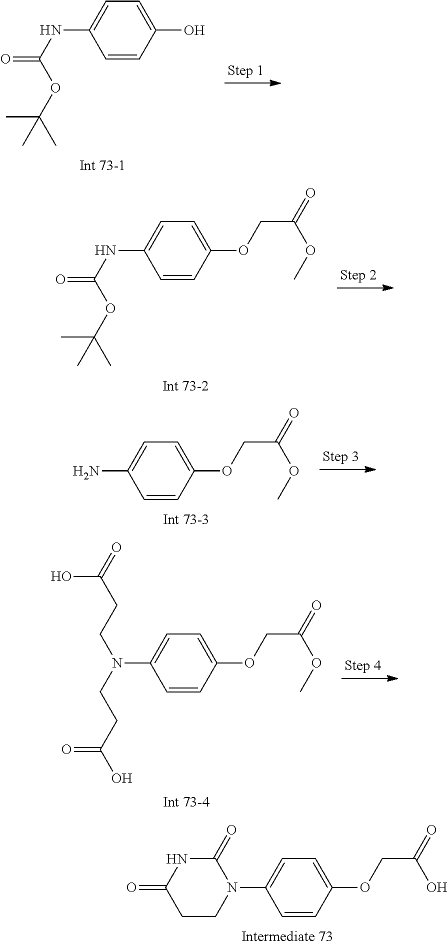 [SMILES: CC(C)(C)OC(=O)Nc1ccc(O)cc1 ; COC(=O)COc1ccc(NC(=O)OC(C)(C)C)cc1 ; COC(=O)COc1ccc(N)cc1 ; COC(=O)COc1ccc(N(CCC(=O)O)CCC(=O)O)cc1 ; O=C(O)COc1ccc(N2CCC(=O)NC2=O)cc1]

### Step 1: methyl 2-(4-((tert-butoxycarbonyl)amino)phenoxy)acetate (Int 73-2)

To a solution of tert-butyl (4-hydroxyphenyl)carbamate (Int 73-1, 7 g, 31.8 mmol) in acetone (75 ml) were added solid Cs₂CO₃ (11.4 g, 35 mmol) and KI (50 mg, 0.301 mmol). Methyl bromoacetate (3 ml, 32.6 mmol) was added and the RM was stirred at reflux for 4 h. The mixture was cooled to RT, filtered and the filtrate was concentrated. The residue was dissolved in EtOAc and the organic phase was washed with a sat. aq. solution of NaHCO₃, dried over MgSO₄ and concentrated. The residue was purified by chromatography on silica gel eluting with EtOAc (from 10% to 25%) in CHX, yielding the title compound methyl 2-(4-((tert-butoxycarbonyl)amino)phenoxy)acetate, Int 73-2, as a solid (8.83 g).

Method LCMS_PL1: Rt=0.97 min; [M+H]⁺=282.

### Step 2: methyl 2-(4-aminophenoxy)acetate (Int 73-3)

To a solution of methyl 2-(4-((tert-butoxycarbonyl)amino)phenoxy)acetate (Int 73-2, 8.83 g, 31.4 mmol) in 1,4-dioxane (30 ml) was added TFA (30 ml) and the RM was stirred at RT overnight. The mixture was concentrated, and the residue was dissolved in DCM. The organic phase was washed with a sat. aq. solution of NaHCO₃, dried over MgSO₄ and concentrated, yielding the title compound methyl 2-(4-aminophenoxy)acetate, Int 73-3, as an oil (5.35 g).

Method LCMS_PL1: Rt=0.37 min; [M+H]⁺=182.

### Step 3: 3,3′-((4-(2-methoxy-2-oxoethoxy)phenyl)azanediyl)dipropanoic Acid (Int 73-4)

To a solution of methyl 2-(4-aminophenoxy)acetate (Int 73-3, 5.347 g, 25.7 mmol) in water (5 ml) at RT was added acrylic acid (11 ml, 160 mmol) and the RM was stirred at 70° C. for 90 min. The mixture was cooled to RT, adsorbed on silica gel and purified by chromatography on silica gel eluting with iPrOH (from 0% to 10%) in DCM, yielding the title compound 3,3′-((4-(2-methoxy-2-oxoethoxy)phenyl)azanediyl)dipropanoic acid, Int 73-4, as a solid (8.24 g).

Method LCMS_PL1: Rt=0.47 min; [M+H]⁺=326.

### Step 4: 2-(4-(2,4-Dioxotetrahydropyrimidin-1(2H)-yl)phenoxy)acetic Acid (Intermediate 73)

A suspension of 3,3′-((4-(2-methoxy-2-oxoethoxy)phenyl)azanediyl)dipropanoic acid (Int 73-4, 8.243 g, 25.09 mmol) and urea (2.260 g, 37.6 mmol) in HOAc (60 ml) was stirred at 120° C. overnight. The mixture was cooled to 0° C. and filtered, the solids were washed with cold water, yielding the title compound 2-(4-(2,4-dioxotetrahydropyrimidin-1(2H)-yl)phenoxy)acetic acid, Intermediate 73, as a solid (4.93 g).

Method LCMS_PL2: Rt=0.75 min; [M+H]⁺=265.

### Intermediate 74: 2-(3-(2,4-dioxotetrahydropyrimidin-1(2H)-yl)-4-methylphenoxy)acetic Acid

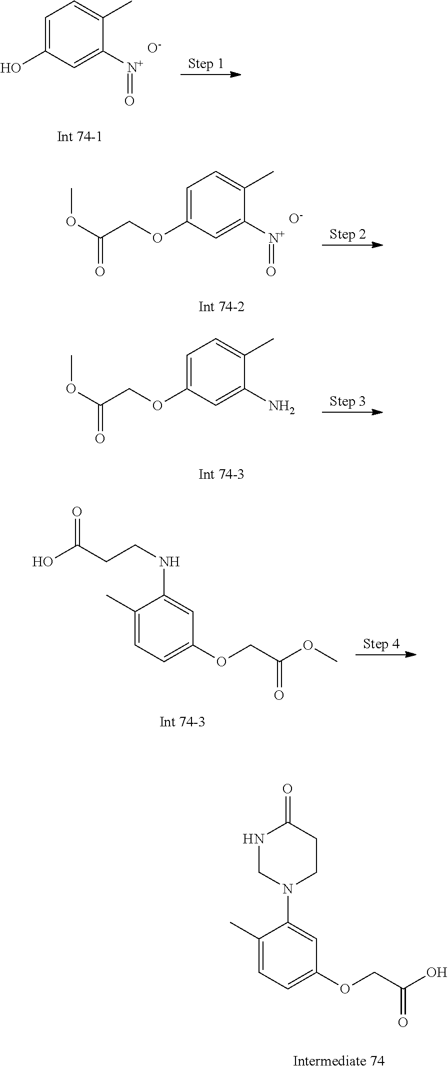

### Step 1: methyl 2-(4-methyl-3-nitrophenoxy)acetate (Int 74-2)

To a solution of 4-methyl-3-nitrophenol (Int 74-1, 6.34 g, 41.4 mmol) in acetone (140 ml) was added solid Cs₂CO₃ (20.23 g, 62.2 mmol). Methyl bromoacetate (5.10 ml, 53.8 mmol) was added and the RM was stirred at 50° C. for 1 h. The mixture was cooled to RT, diluted with water and the aq. phase was extracted with Et₂O. The combined organic phases were washed with brine, dried over MgSO₄ and concentrated. The residue was purified by chromatography on silica gel eluting with MeOH (from 0% to 12.5%) in DCM, yielding the title compound methyl 2-(4-methyl-3-nitrophenoxy)acetate, Int 74-2, as a solid (1.183 g).

¹H NMR (400 MHz, DMSO-d₆) δ 7.51 (d, J=2.8 Hz, 1H), 7.40 (d, J=8.5 Hz, 1H), 7.24 (dd, J=8.5, 2.9 Hz, 1H), 4.90 (s, 2H), 3.69 (s, 3H), 2.41 (s, 3H).

### Step 2: methyl 2-(3-amino-4-methylphenoxy)acetate (Int 74-3)

To a solution of methyl 2-(4-methyl-3-nitrophenoxy)acetate (Int 74-2, 9.120 g, 39.6 mmol) in MeOH (100 ml) under an argon atmosphere was added Pd/C (10%, 421 mg, 0.396 mmol) and the RM was stirred under a hydrogen atmosphere at RT for 18 h. The mixture was filtered through CELITE©, the solids were washed with MeOH and the filtrate was concentrated, yielding the title compound methyl 2-(3-amino-4-methylphenoxy)acetate, Int 74-3, as a solid (7.411 g), which was used for the next step without further purification.

Method LCMS_PL1: Rt=0.70 min; [M+H]⁺=196.

### Step 3: 3-((5-(2-methoxy-2-oxoethoxy)-2-methylphenyl)amino)propanoic Acid (Int 74-4)

To a mixture of methyl 2-(3-amino-4-methylphenoxy)acetate (Int 74-3, 7.350 g, 35.0 mmol) in water (10 ml) was added acrylic acid (15 ml, 219 mmol) at RT and the RM was stirred at 70° C. overnight. The mixture was concentrated, the residue was adsorbed on ISOLUTE© and purified by chromatography on silica gel eluting with MeOH (from 0% to 25%) in DCM, yielding the title compound 3-((5-(2-methoxy-2-oxoethoxy)-2-methylphenyl)amino)propanoic acid, Int 74-4, as an oil (23.7 g).

Method LCMS_PL1: Rt=0.76 min; [M+H]⁺=268.

### Step 4: 2-(3-(2,4-dioxotetrahydropyrimidin-1(2H)-yl)-4-methylphenoxy)acetic acid (Intermediate 74)

A mixture of 3-((5-(2-methoxy-2-oxoethoxy)-2-methylphenyl)amino)propanoic acid (Int 74-4, 23.7 g, 35.00 mmol) and urea (3.15 g, 52.5 mmol) in HOAc (60 ml) was stirred under an argon atmosphere at 120° C. overnight. An aq. solution of HCl (4 M, 50 ml) was added and the RM was refluxed for 45 min. The mixture was cooled to 0° C. and filtered. The solids were washed with MTBE, yielding the title compound 2-(3-(2,4-dioxotetrahydropyrimidin-1(2H)-yl)-4-methylphenoxy)acetic acid, Intermediate 74, as a solid (4.31 g).

Method LCMS_PL1: Rt=0.49 min; [M+H]⁺=279.

### Intermediate 75: 1-((1-(2-(4-(((3R,4R)-3-fluoropiperidin-4-yl)oxy)piperidin-1-yl)ethyl)-2-oxo-1,2-dihydropyridin-3-yl)methyl)dihydropyrimidine-2,4(1H,3H)-dione

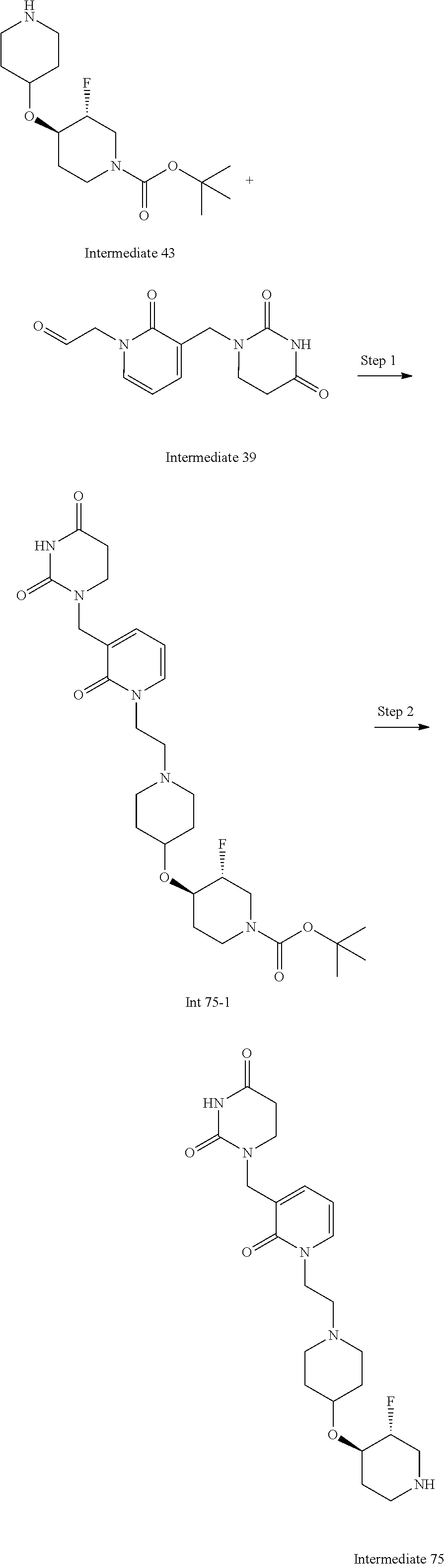 [SMILES: CC(C)(C)OC(=O)N1CC[C@@H](OC2CCNCC2)[C@H](F)C1 ; O=CCn1cccc(CN2CCC(=O)NC2=O)c1=O ; CC(C)(C)OC(=O)N1CC[C@@H](OC2CCN(CCn3cccc(CN4CCC(=O)NC4=O)c3=O)CC2)[C@H](F)C1 ; O=C1CCN(Cc2cccn(CCN3CCC(O[C@@H]4CCNC[C@H]4F)CC3)c2=O)C(=O)N1]

### Step 1: tert-butyl (3R,4R)-4-((1-(2-(3-((2,4-dioxotetrahydropyrimidin-1(2H)-yl)methyl)-2-oxopyridin-1(2H)-yl)ethyl)piperidin-4-yl)oxy)-3-fluoropiperidine-1-carboxylate (Int 75-1)

To a solution of tert-butyl (3R,4R)-3-fluoro-4-(piperidin-4-yloxy)piperidine-1-carboxylate 4-methylbenzenesulfonate salt (Intermediate 43, 169 mg, 0.34 mmol), TEA (150 μl, 1.076 mmol), 2-(3-((2,4-dioxotetrahydropyrimidin-1(2H)-yl)methyl)-2-oxopyridin-1(2H)-yl)acetaldehyde (Intermediate 39, 109 mg, 0.372 mmol) in a mixture of MeOH (1.5 ml) and DCM (1.5 ml) were added MgSO₄ (407 mg, 3.38 mmol) and a solution of ZnCl₂ (0.7 M) in THE (0.725 ml, 0.51 mmol) and the RM was stirred at RT for 24 h. Solid NaBH₃CN (40 mg, 0.64 mmol) was added and the RM was stirred at RT for 24 h. The mixture was filtered through CELITE® and the solids were washed with DCM. The filtrate was concentrated and the residue was purified by reverse phase chromatography on a REDISEP® Gold HP C18 column (15.5 g) eluting with ACN (from 2% to 100%) in an aq. solution of TFA (0.1%), yielding the title compound tert-butyl (3R,4R)-4-((1-(2-(3-((2,4-dioxotetrahydropyrimidin-1(2H)-yl)methyl)-2-oxopyridin-1(2H)-yl)ethyl)piperidin-4-yl)oxy)-3-fluoropiperidine-1-carboxylate, Int 75-1, as a solid TFA salt (248 mg).

Method LCMS_MLG9: Rt=0.67 min; [M+H]⁺=550.

### Step 2: 1-((1-(2-(4-(((3R,4R)-3-fluoropiperidin-4-yl)oxy)piperidin-1-yl)ethyl)-2-oxo-1,2-dihydropyridin-3-yl)methyl)dihydropyrimidine-2,4(1H,3H)-dione (Intermediate 75)

To a solution of tert-butyl (3R,4R)-4-((1-(2-(3-((2,4-dioxotetrahydropyrimidin-1(2H)-yl)methyl)-2-oxopyridin-1(2H)-yl)ethyl)piperidin-4-yl)oxy)-3-fluoropiperidine-1-carboxylate TFA salt (Int 75-1, 248 mg, 0.280 mmol) in DCM (2.5 ml) was added TFA (650 ul, 8.44 mmol) and the RM was stirred at RT for 1 h. The mixture was concentrated and the residue was purified by reverse phase chromatography on a REDISEP® Gold HP C18 column (15.5 g) eluting with ACN (from 2% to 100%) in an aq. solution of TFA (0.1%), yielding the title compound 1-((1-(2-(4-(((3R,4R)-3-fluoropiperidin-4-yl)oxy)piperidin-1-yl)ethyl)-2-oxo-1,2-dihydropyridin-3-yl)methyl)dihydropyrimidine-2,4(1H,3H)-dione, Intermediate 75, as a solid TFA salt (199 mg).

Method LCMS_MLG9: Rt=0.31 min; [M+H]⁺=450.

### Example 2. Synthesis of Final Compounds

### Compound A1: 1-(5-(4-((1-(4-(1,5-Dimethyl-6-oxo-1,6-dihydropyridin-3-yl)-2,6-dimethoxybenzyl)piperidin-4-yl)oxy)piperidine-1-carbonyl)-2-methoxyphenyldihydropyrimidine-2,4(1H,3H)-dione

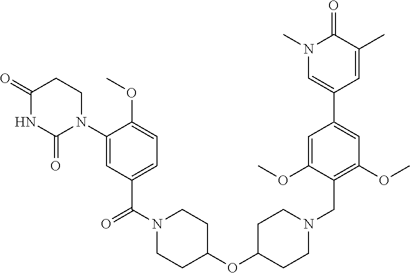 [SMILES: COc1ccc(C(=O)N2CCC(OC3CCN(Cc4c(OC)cc(-c5cc(C)c(=O)n(C)c5)cc4OC)CC3)CC2)cc1N1CCC(=O)NC1=O]

To a 10 ml round bottom flask were added 1-(2-methoxy-5-(4-(piperidin-4-yloxy)piperidine-1-carbonyl)phenyl)dihydropyrimidine-2,4(1H,3H)-dione hydrochloride (intermediate 29,141 mg, 0.274 mmol), HOAc (0.014 ml, 0.248 mmol), NaOAc (64 mg, 0.780 mmol) and DCM (2 ml). The mixture was stirred at 0° C. for 10 min, 4-(1,5-dimethyl-6-oxo-1,6-dihydropyridin-3-yl)-2,6-dimethoxybenzaldehyde (intermediate 9, 75 mg, 0.261 mmol) was added and the mixture was stirred at RT for 30 min. Solid NaBH(OAc)₃ (111 mg, 0.552 mmol) was added and the RM was stirred at RT overnight. Further solid NaBH(OAc)₃ (55 mg, 0.260 mmol) was added and the RM was stirred at RT for 2 h. The RM was filtered over CELITE®, the solids were washed with MeOH and the combined filtrates were evaporated. The residue was purified by reversed phase chromatography on a REDISEP® Gold HP C18 column (15.5 g) eluting with ACN (from 2% to 100%) in aq. NH₄HCO₃ (0.1%). The obtained material was repurified by chromatography on silica gel eluting with a mixture (4:1) of DCM and iPrOH containing 1% of NH₄OH (from 0% to 100%) in DCM. The obtained material was repurified by SFC on a Reprospher PEI column (250×30 mm, 100 Å, 5 m) eluting with MeOH (from 21% to 29%) in CO₂, yielding the title compound as a solid (37 mg).

Method L: Rt=2.97 min; [M+H]⁺=702.

¹H NMR (400 MHz, DMSO-d₆) δ 10.32 (s, 1H), 8.03 (d, J=2.6 Hz, 1H), 7.80 (s, 1H), 7.37 (dd, J=8.5, 2.1 Hz, 1H), 7.33 (d, J=2.0 Hz, 1H), 7.14 (d, J=8.5 Hz, 1H), 6.79 (s, 2H), 3.83 (m, 9H), 3.68-3.41 (m, 11H), 3.19 (m, 2H), 2.67 (m, 4H), 2.09 (m, 5H), 1.75 (m, 4H), 1.38 (m, 4H).

### Compound A2: 1-(5-(4-((1-(4-(1,5-Dimethyl-6-oxo-1,6-dihydropyridin-3-yl)-2-methoxybenzoyl)piperidin-4-yl)oxy)piperidine-1-carbonyl)-2-methoxyphenyl)dihydropyrimidine-2,4(1H,3H)-dione

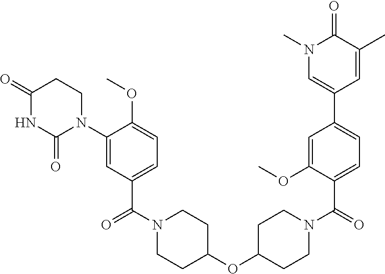 [SMILES: COc1cc(-c2cc(C)c(=O)n(C)c2)ccc1C(=O)N1CCC(OC2CCN(C(=O)c3ccc(OC)c(N4CCC(=O)NC4=O)c3)CC2)CC1]

To a 50 ml round bottom flask were added HATU (72 mg, 0.189 mmol), 4-(1,5-dimethyl-6-oxo-1,6-dihydropyridin-3-yl)-2-methoxybenzoic acid (intermediate 10, 40 mg, 0.146 mmol), DIPEA (0.100 ml, 0.573 mmol) and DMF (1 ml). The RM was stirred at RT for 30 min, a solution of 1-(2-methoxy-5-(4-(piperidin-4-yloxy)piperidine-1-carbonyl)phenyl)dihydropyrimidine-2,4(1H,3H)-dione hydrochloride (intermediate 29, 90 mg, 0.176 mmol) was added and the RM was stirred at RT overnight. The mixture was directly purified by reversed phase chromatography on a REDISEP® Gold HP C18 column (15.5 g) eluting with ACN (from 2% to 100%) in an aq. solution of TFA (0.1%), followed by a purification using SFC on a Reprospher PEI column (250×30 mm, 100 Å, 5 m) eluting with MeOH (from 20% to 30%) in CO₂, yielding the title compound as a solid (37 mg).

Method L: Rt=3.37 min; [M+H]⁺=686.

¹H NMR (400 MHz, DMSO-d₆) δ 10.32 (s, 1H), 8.06 (s, 1H), 7.80 (s, 1H), 7.45-7.31 (m, 2H), 7.18 (m, 4H), 3.99 (m, 2H), 3.89-3.81 (m, 6H), 3.71 (m, 2H), 3.59 (t, J=6.7 Hz, 2H), 3.53 (m, 3H), 3.12 (m, 6H), 2.68 (t, J=6.8 Hz, 2H), 2.09 (s, 3H), 1.77 (m, 4H), 1.36 (m, 4H).

### Compound A3: 1-(5-(4-((4-(4-(1,5-Dimethyl-6-oxo-1,6-dihydropyridin-3-yl)-2-methoxybenzoyl)piperazin-1-yl)methyl)piperidine-1-carbonyl)-2-methoxyphenyl)dihydropyrimidine-24(1H,3H)-dione

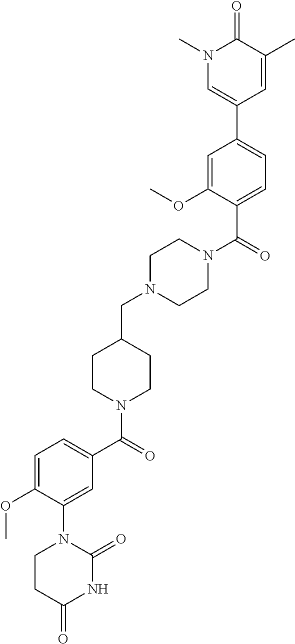 [SMILES: COc1cc(-c2cc(C)c(=O)n(C)c2)ccc1C(=O)N1CCN(CC2CCN(C(=O)c3ccc(OC)c(N4CCC(=O)NC4=O)c3)CC2)CC1]

### Step 1. tert-butyl 4-(4-(1,5-dimethyl-6-oxo-1,6-dihydropyridin-3-yl)-2-methoxybenzoyl)piperazine-1-carboxylate

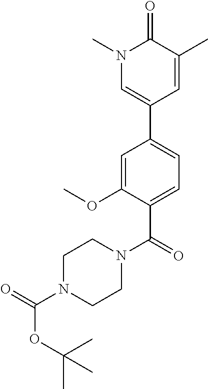 [SMILES: COc1cc(-c2cc(C)c(=O)n(C)c2)ccc1C(=O)N1CCN(C(=O)OC(C)(C)C)CC1]

To a 50 ml round bottom flask were added HATU (265 mg, 0.698 mmol), 4-(1,5-dimethyl-6-oxo-1,6-dihydropyridin-3-yl)-2-methoxybenzoic acid (intermediate 10, 159 mg, 0.582 mmol), DIPEA (0.250 ml, 1.431 mmol) and DMF (5 ml). The RM was stirred at RT for 30 min, tert-butyl piperazine-1-carboxylate (130 mg, 0.698 mmol) and DIPEA (0.250 ml, 1.431 mmol) were added and the RM was stirred at RT overnight. The mixture was directly purified by reversed phase chromatography on a REDISEP® Gold HP C18 column (50 g) eluting with ACN (from 2% to 100%) in an aq. solution of NH₄HCO₃ (0.1%), yielding the title compound as a solid (281 mg).

Method D: Rt=0.92 min; [M+H]⁺=442.

### Step 2: 5-(3-methoxy-4-(piperazine-1-carbonyl)phenyl)-1,3-dimethylpyridin-2(1H)-one

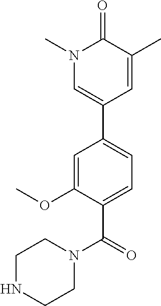

To a 25 ml round bottom flask were added tert-butyl 4-(4-(1,5-dimethyl-6-oxo-1,6-dihydropyridin-3-yl)-2-methoxybenzoyl)piperazine-1-carboxylate (281 mg, 0.579 mmol), TFA (2 ml, 26.0 mmol) and DCM (5 ml). The RM was stirred at RT for 1 h and concentrated, the residue was diluted in a mixture of water and ACN and freeze dried, yielding the corresponding TFA salt of the title compound as a solid (266 mg).

Method D: Rt=0.41 min; [M+H]⁺=342.

### Step 3: tert-butyl 4-((4-(4-(1,5-dimethyl-6-oxo-1,6-dihydropyridin-3-yl)-2-methoxybenzoyl)piperazin-1-yl)methyl)piperidine-1-carboxylate

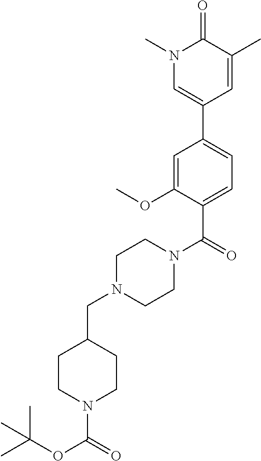 [SMILES: COc1cc(-c2cc(C)c(=O)n(C)c2)ccc1C(=O)N1CCN(CC2CCN(C(=O)OC(C)(C)C)CC2)CC1]

To a 10 ml round bottom flask were added 5-(3-methoxy-4-(piperazine-1-carbonyl)phenyl)-1,3-dimethylpyridin-2(1H)-one TFA salt (262 mg, 0.570 mmol), tert-butyl 4-formylpiperidine-1-carboxylate (146 mg, 0.684 mmol), TEA (0.300 ml, 2.152 mmol), a solution of ZnCl₂ (0.5 M) in THE (1.2 ml, 0.600 mmol) and MeOH (4 ml). The RM was stirred at RT for 7 h, solid NaBH₃CN (40 mg, 0.637 mmol) was added and the RM was stirred at RT for 2 days.

The mixture was concentrated and the residue was purified by chromatography on silica gel eluting with MeOH (from 0% to 7%) in DCM, yielding the title compound as a solid (341 mg).

Method D: Rt=0.71 min; [M-tBu+H]⁺=483.

### Step 4: 5-(3-methoxy-4-(4-(piperidin-4-ylmethyl)piperazine-1-carbonyl)phenyl)-1,3-dimethylpyridin-2(1H)-one

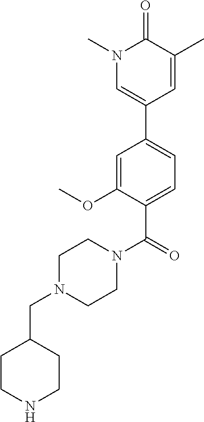 [SMILES: COc1cc(-c2cc(C)c(=O)n(C)c2)ccc1C(=O)N1CCN(CC2CCNCC2)CC1]

To a 25 ml round bottom flask were added tert-butyl 4-((4-(4-(1,5-dimethyl-6-oxo-1,6-dihydropyridin-3-yl)-2-methoxybenzoyl)piperazin-1-yl)methyl)piperidine-1-carboxylate (341 mg, 0.570 mmol), TFA (2 ml, 26.0 mmol) and DCM (5 ml). The RM was stirred at RT for 1 h, concentrated and the residue was purified by reversed phase chromatography on a REDISEP® Gold HP C18 column (50 g) eluting with ACN (from 2% to 100%) in an aq. solution of TFA (0.1%), yielding the corresponding TFA salt of the title compound as a solid (372 mg).

Method D: Rt=0.38 min; [M+H]⁺=439.

### Step 5: 1-(5-(4-((4-(4-(1,5-dimethyl-6-oxo-1,6-dihydropyridin-3-yl)-2-methoxybenzoyl)piperazin-1-yl)methyl)piperidine-1-carbonyl)-2-methoxyphenyl)dihydropyrimidine-2,4(1H,3H)-dione

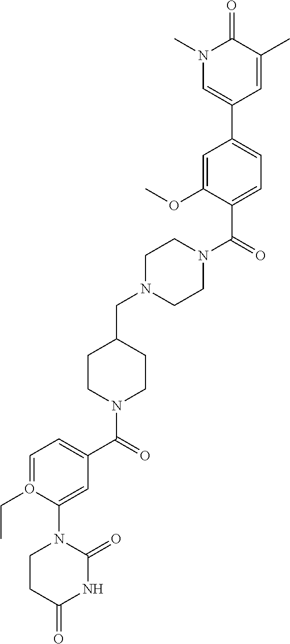

To a 50 ml round bottom flask were added HATU (121 mg, 0.318 mmol), 3-(2,4-dioxotetrahydropyrimidin-1(2H)-yl)-4-methoxybenzoic acid (intermediate 25, 77 mg, 0.291 mmol), DIPEA (0.150 ml, 0.859 mmol) and DMF (1.5 ml). The RM was stirred at RT for 30 min, a solution of 5-(3-methoxy-4-(4-(piperidin-4-ylmethyl)piperazine-1-carbonyl)phenyl)-1,3-dimethylpyridin-2(1H)-one TFA salt (180 mg, 0.265 mmol) and DIPEA (0.150 ml, 0.859 mmol) in DMF (0.5 ml) was added and the RM was stirred at RT for 4 days. The mixture was directly purified by reversed phase chromatography on a REDISEP® Gold HP C18 column (15.5 g) eluting with ACN (from 2% to 100%) in an aq. solution of TFA (0.1%), the compound containing fractions were pooled, filtered over a PL-HCO₃ MP SPE cartridge and the filtrate was freeze dried, yielding the title compound as a solid (147 mg).

Method L: Rt=2.19 min; [M+H]⁺=685.

¹H NMR (400 MHz, DMSO-d₆) δ 10.32 (s, 1H), 8.06 (d, J=2.6 Hz, 1H), 7.80 (d, J=2.5 Hz, 1H), 7.35 (dd, J=8.5, 2.1 Hz, 1H), 7.31 (d, J=2.1 Hz, 1H), 7.22-7.11 (m, 4H), 3.85 (m, 6H), 3.55 (m, 9H), 3.15 (m, 4H), 2.69 (m, 2H), 2.40-2.07 (m, 9H), 1.75 (m, 3H), 1.15-0.99 (m, 2H).

### Compound A4: 1-(2-Methoxy-5-(4-((4-(2-methoxy-4-(2-methyl-1-oxo-1,2-dihydro-2,7-naphthyridin-4-yl)phenoxy)piperidin-1-yl)methyl)piperidine-1-carbonyl)phenyl)dihydropyrimidine-2,4(1H,3H)-dione

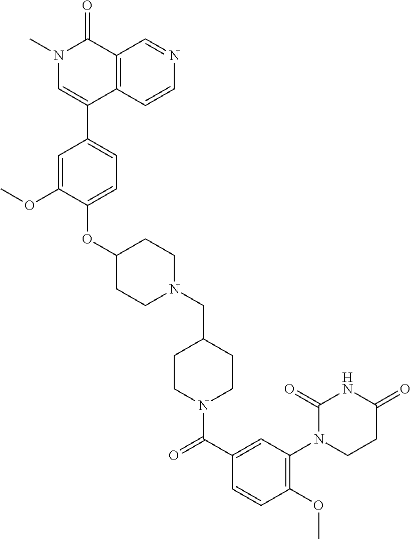 [SMILES: COc1cc(-c2cn(C)c(=O)c3cnccc23)ccc1OC1CCN(CC2CCN(C(=O)c3ccc(OC)c(N4CCC(=O)NC4=O)c3)CC2)CC1]

To a 50 ml round bottom flask were added HATU (56 mg, 0.147 mmol), 3-(2,4-dioxotetrahydropyrimidin-1(2H)-yl)-4-methoxybenzoic acid (intermediate 25, 39 mg, 0.148 mmol), DIPEA (0.100 ml, 0.573 mmol) and DMF (0.5 ml). The RM was stirred at RT for 30 min, a solution of 4-(3-methoxy-4-((1-(piperidin-4-ylmethyl)piperidin-4-yl)oxy)phenyl)-2-methyl-2,7-naphthyridin-1(2H)-one TFA salt (intermediate 20, 100 mg, 0.122 mmol), DIPEA (0.100 ml, 0.573 mmol) and DMF (0.5 ml) was added and the RM was stirred at RT overnight. The mixture was directly purified by reversed phase chromatography on a REDISEP® Gold HP C18 column (15.5 g) eluting with ACN (from 2% to 90%) in an aq. solution of TFA (0.1%), the fractions containing the compound were pooled and filtered through a PL-HCO₃ MP SPE cartridge. The resulting material was then purified by SFC on a Princeton PPU column (250×30 mm, 5 m, 100 Å) eluting with MeOH (from 28% to 38%) in CO₂, yielding the title compound as a solid (84.2 mg).

Method L: Rt=2.47 min; [M+H]⁺=709.

¹H NMR (400 MHz, DMSO-d₆) δ 10.33 (s, 1H), 9.43 (s, 1H), 8.71 (d, J=5.7 Hz, 1H), 7.78 (s, 1H), 7.48 (d, J=5.7 Hz, 1H), 7.37 (dd, J=8.4, 2.1 Hz, 1H), 7.32 (d, J=2.1 Hz, 1H), 7.20-7.09 (m, 2H), 7.05 (s, 1H), 6.95 (d, J=8.1 Hz, 1H), 4.35 (m, 1H), 3.85 (s, 3H), 3.81 (s, 3H), 3.61 (m, 5H), 2.84 (m, 4H), 2.68 (m, 2H), 2.50 (m, 2H), 2.17 (m, 4H), 1.97 (m, 2H), 1.71 (m, 5H), 1.09 (m, 2H).

### Compound A5: 1-(2-Methoxy-5-(4-((4-((2-methoxy-4-(2-methyl-1-oxo-1,2-dihydro-2,7-naphthyridin-4-yl)phenoxy)methyl)piperidin-1-yl)methyl)piperidine-1-carbonyl)phenyl)dihydropyrimidine-2,4(1H,3H)-dione

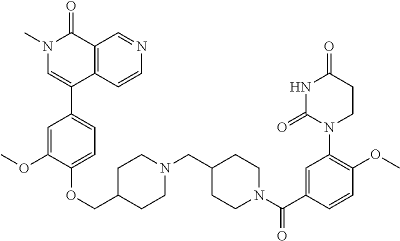

### Step 1: tert-butyl 4-((2-methoxy-4-(4,4,5,5-tetramethyl-1,3,2-dioxaborolan-2-yl)phenoxy)methyl)piperidine-1-carboxylate

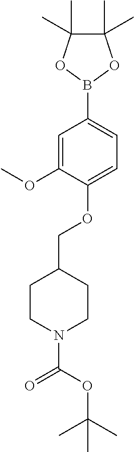

To a 100 ml two-necked flask were added under an argon atmosphere 2-methoxy-4-(4,4,5,5-tetramethyl-1,3,2-dioxaborolan-2-yl)phenol (1.48 g, 5.92 mmol), tert-butyl 4-(hydroxymethyl)piperidine-1-carboxylate (1.43 g, 6.51 mmol), PPh₃ (1.86 g, 7.09 mmol) and THE (30 ml). DIAD (1.4 ml, 7.20 mmol) was added dropwise and the RM was stirred at RT overnight. The RM was added into a mixture of EtOAc and an aq. sat. solution of NaHCO₃, the aq. phase was extracted with EtOAc, the combined organic phases were washed with brine, dried over MgSO₄ and the residue was purified by chromatography on silica gel eluting with EtOAc (from 0% to 20%) in CHX, yielding the title compound as a solid (2.23 g).

Method D: Rt=1.45 min; [M+H]⁺=448.

### Step 2: tert-butyl 4-((2-methoxy-4-(2-methyl-1-oxo-1,2-dihydro-2,7-naphthyridin-4-yl)phenoxy)methyl)piperidine-1-carboxylate

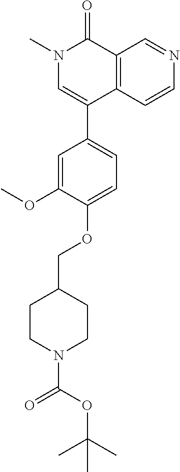 [SMILES: COc1cc(-c2cn(C)c(=O)c3cnccc23)ccc1OCC1CCN(C(=O)OC(C)(C)C)CC1]

To a 50 ml round bottom flask were added under an argon atmosphere 4-bromo-2-methyl-2,7-naphthyridin-1(2H)-one (intermediate 5, 190 mg, 2.05 mmol), tert-butyl 4-((2-methoxy-4-(4,4,5,5-tetramethyl-1,3,2-dioxaborolan-2-yl)phenoxy)methyl)piperidine-1-carboxylate (1.0 g, 2.235 mmol), K₂CO₃ (850 mg, 6.15 mmol), ACN (16 ml) and water (4 ml). Solid PdCl₂(dppf)-CH₂Cl₂ (124 mg, 0.167 mmol) was added and the RM was stirred at 100° C. for 1 h. The mixture was cooled to RT and filtered through CELITE®, the filtrate was concentrated and diluted with Et₂O. The resulting mixture was filtered and the solids were dried, yielding the title compound as a solid (577 mg).

Method D: Rt=1.14 min; [M+H]⁺=480.

### Step 3: 4-(3-methoxy-4-(piperidin-4-ylmethoxy)phenyl)-2-methyl-2,7-naphthyridin-1(2H)-one

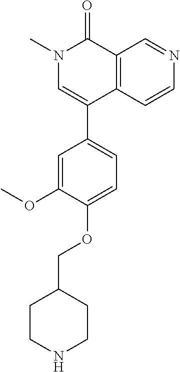 [SMILES: COc1cc(-c2cn(C)c(=O)c3cnccc23)ccc1OCC1CCNCC1]

To a 25 ml round bottom flask were added tert-butyl 4-((2-methoxy-4-(2-methyl-1-oxo-1,2-dihydro-2,7-naphthyridin-4-yl)phenoxy)methyl)piperidine-1-carboxylate (220 mg, 0.408 mmol), TFA (1 ml, 12.98 mmol) and DCM (1 ml). The RM was stirred at RT for 1 h, concentrated and the residue was purified by reversed phase chromatography on a REDISEP® Gold HP C18 column (50 g) eluting with ACN (from 2% to 100%) in an aq. solution of TFA (0.1%), yielding the corresponding TFA salt of the title compound as a solid (137 mg).

Method D: Rt=0.54 min; [M+H]⁺=380.

### Step 4. tert-butyl 4-((4-((2-methoxy-4-(2-methyl-1-oxo-1,2-dihydro-2,7-naphthyridin-4-yl)phenoxy)methyl)piperidin-1-yl)methyl)piperidine-1-carboxylate

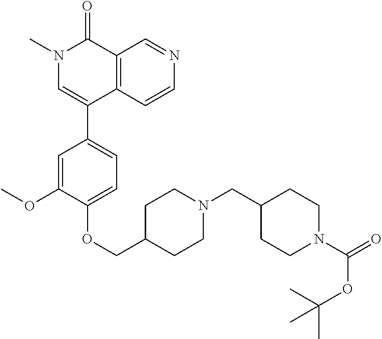 [SMILES: COc1cc(-c2cn(C)c(=O)c3cnccc23)ccc1OCC1CCN(CC2CCN(C(=O)OC(C)(C)C)CC2)CC1]

To a 10 ml round bottom flask were added 4-(3-methoxy-4-(piperidin-4-ylmethoxy)phenyl)-2-methyl-2,7-naphthyridin-1(2H)-one TFA salt (134 mg, 0.250 mmol), tert-butyl 4-formylpiperidine-1-carboxylate (60 mg, 0.281 mmol), TEA (0.100 ml, 0.717 mmol), a solution of ZnCl₂ (0.5 M) in THF (0.550 ml, 0.275 mmol) and MeOH (1.5 ml). The RM was stirred at RT for 7 h, solid NaBH₃CN (17 mg, 0.271 mmol) was added and the RM was stirred at RT for 2 days. The mixture was concentrated and the residue was purified by reversed phase chromatography on a REDISEP® Gold HP C18 column (15.5 g) eluting with ACN (from 2% to 100%) in an aq. solution of TFA (0.1%), yielding the corresponding TFA salt of the title compound as a solid (173 mg).

Method D: Rt=0.78 min; [M+H]⁺=577.

### Step 5:4-(3-methoxy-4-((1-(piperidin-4-ylmethyl)piperidin-4-yl)methoxy)phenyl)-2-methyl-2,7-naphthyridin-1(2H)-one

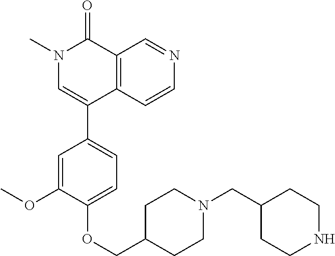 [SMILES: COc1cc(-c2cn(C)c(=O)c3cnccc23)ccc1OCC1CCN(CC2CCNCC2)CC1]

To a 10 ml round bottom flask were added tert-butyl 4-((4-((2-methoxy-4-(2-methyl-1-oxo-1,2-dihydro-2,7-naphthyridin-4-yl)phenoxy)methyl)piperidin-1-yl)methyl)piperidine-1-carboxylate TFA salt (162 mg, 0.232 mmol), TFA (0.5 ml, 6.49 mmol) and DCM (1 ml). The RM was stirred at RT for 2 h and concentrated, the residue was diluted in a mixture of ACN and water and freeze dried, yielding the corresponding TFA salt of the title compound as a solid (169 mg).

Method D: Rt=0.43 min; [M+H]⁺=477.

### Step 6:1-(2-methoxy-5-(4-((4-((2-methoxy-4-(2-methyl-1-oxo-1,2-dihydro-2,7-naphthyridin-4-yl)phenoxy)methyl)piperidin-1-yl)methyl)piperidine-1-carbonyl)phenyl)dihydropyrimidine-2,4(1H,3H)-dione

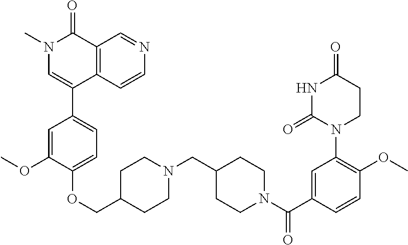

To a 50 ml round bottom flask were added HATU (96 mg, 0.253 mmol), 3-(2,4-dioxotetrahydropyrimidin-1(2H)-yl)-4-methoxybenzoic acid (intermediate 25, 67 mg, 0.257 mmol), DIPEA (0.100 ml, 0.573 mmol) and DMF (1.5 ml). The RM was stirred at RT for 30 min, a solution of 4-(3-methoxy-4-((1-(piperidin-4-ylmethyl)piperidin-4-yl)methoxy)phenyl)-2-methyl-2,7-naphthyridin-1(2H)-one TFA salt (167 mg, 0.230 mmol), DIPEA (0.100 ml, 0.573 mmol) and DMF (0.5 ml) was added and the RM was stirred at RT overnight. The mixture was directly purified by reversed phase chromatography on a REDISEP® Gold HP C18 column (15.5 g) eluting with ACN (from 2% to 100%) in an aq. solution of NH₄HCO₃ (0.1%), followed by a purification using SFC on a Reprospher PEI column (250×30 m, 100 Å, 5 m) eluting with MeOH (from 24% to 32%) in CO₂, yielding the title compound as a solid (43 mg).

Method L: Rt=2.62 min; [M+H]⁺=723.

¹H NMR (400 MHz, DMSO-d₆) δ 10.33 (s, 1H), 9.43 (s, 1H), 8.70 (d, J=5.6 Hz, 1H), 7.76 (s, 1H), 7.47 (d, J=5.7 Hz, 1H), 7.36 (dd, J=8.5, 2.2 Hz, 1H), 7.32 (d, J=2.1 Hz, 1H), 7.15 (d, J=8.5 Hz, 1H), 7.07 (d, J=8.3 Hz, 1H), 7.02 (d, J=2.0 Hz, 1H), 6.95 (dd, J=8.2, 2.1 Hz, 1H), 4.38 (m, 2H), 3.92-3.73 (m, 8H), 3.59 (m, 5H), 2.87 (m, 4H), 2.68 (t, J=6.5 Hz, 2H), 2.15 (d, J=6.9 Hz, 2H), 1.79 (m, 8H), 1.38-1.20 (m, 2H), 1.06 (m, 2H).

### Compound A6: 1-(5-(4-((4-(2,6-Dimethoxy-4-(2-methyl-1-oxo-1,2-dihydro-2,7-naphthyridin-4-yl)phenoxy)piperidin-1-yl)methyl)piperidine-1-carbonyl)-2-methoxyphenyl)dihydropyrimidine-2,4(1H,3H)-dione

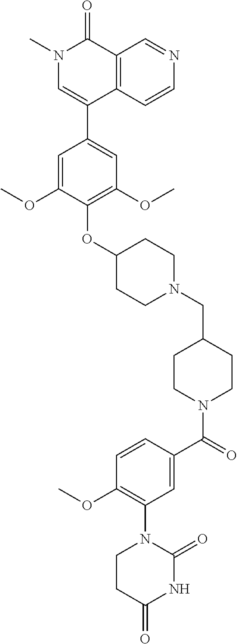 [SMILES: COc1ccc(C(=O)N2CCC(CN3CCC(Oc4c(OC)cc(-c5cn(C)c(=O)c6cnccc56)cc4OC)CC3)CC2)cc1N1CCC(=O)NC1=O]

To a 50 ml round bottom flask were added HATU (71 mg, 0.187 mmol), 3-(2,4-dioxotetrahydropyrimidin-1(2H)-yl)-4-methoxybenzoic acid (intermediate 25, 49 mg, 0.185 mmol), DIPEA (0.100 ml, 0.573 mmol) and DMF (1 ml). The RM was stirred at RT for 30 min, a solution of 4-(3,5-dimethoxy-4-((1-(piperidin-4-ylmethyl)piperidin-4-yl)oxy)phenyl)-2-methyl-2,7-naphthyridin-1(2H)-one TFA salt (intermediate 21, 117 mg, 0.156 mmol), DIPEA (0.100 ml, 0.573 mmol) and DMF (0.5 ml) was added and the RM was stirred at RT overnight. The mixture was directly purified by reversed phase chromatography on a REDISEP® Gold HP C18 column (15.5 g) eluting with ACN (from 2% to 100%) in an aq. solution of NH₄HCO₃ (0.1%), followed by a purification using SFC on a Reprospher PEI column (250×30 mm, 100 Å, 5 m) eluting with MeOH (from 24% to 32%) in CO₂, yielding the title compound as a solid (43 mg).

Method L. Rt=2.64 min; [M+H]⁺=739.

¹H NMR (400 MHz, DMSO-d₆) δ 10.33 (s, 1H), 9.44 (s, 1H), 8.72 (d, J=5.6 Hz, 1H), 7.84 (s, 1H), 7.55 (d, J=5.6 Hz, 1H), 7.41-7.27 (m, 2H), 7.16 (d, J=8.5 Hz, 1H), 6.74 (s, 2H), 4.38 (s, 1H), 4.03 (m, 1H), 3.82 (m, 9H), 3.60 (m, 5H), 3.05-2.62 (m, 7H), 2.20-1.99 (m, 4H), 1.72 (m, 7H), 1.06 (m, 2H).

### Compound A7: 1-(4-(2-(4-((1-(4-(1,5-Dimethyl-6-oxo-1,6-dihydropyridin-3-yl)-2,5-dimethoxybenzyl)piperidin-4-yl)oxy)piperidin-1-yl)-2-oxoethoxy)phenyl)dihydropyrimidine-2,4(1H,3H)-dione

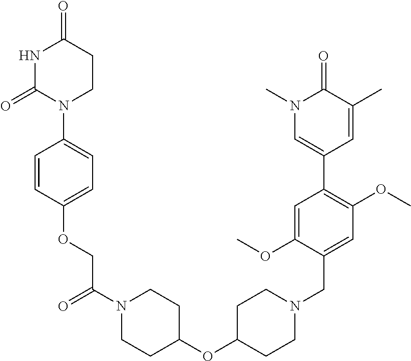 [SMILES: COc1cc(-c2cc(C)c(=O)n(C)c2)c(OC)cc1CN1CCC(OC2CCN(C(=O)COc3ccc(N4CCC(=O)NC4=O)cc3)CC2)CC1]

To a 10 ml round bottom flask were added 1-(4-(2-oxo-2-(4-(piperidin-4-yloxy)piperidin-1-yl)ethoxy)phenyl)dihydropyrimidine-2,4(1H,3H)-dione hydrochloride (intermediate 30, 186 mg, 0.339 mmol), 4-(1,5-dimethyl-6-oxo-1,6-dihydropyridin-3-yl)-2,5-dimethoxybenzaldehyde (intermediate 8, 117 mg, 0.339 mmol), TEA (0.150 ml, 1.076 mmol), a solution of ZnCl₂ (0.5 M) in THE (0.7 ml, 0.350 mmol) and MeOH (2 ml). The RM was stirred at RT for 7 h, solid NaBH₃CN (22 mg, 0.350 mmol) was added and the RM was stirred at RT overnight. The mixture was concentrated and the residue purified by reversed phase chromatography on a REDISEP® Gold HP C18 column (15.5 g) eluting with ACN (from 0% to 100%) in an aq. solution of NH₄HCO₃ (0.1%), followed by a purification using SFC on a Reprospher PEI column (250×30 mm, 100 Å, 5 m) eluting with MeOH (from 24% to 32%) in CO₂, yielding the title compound as a solid (56 mg).

Method L: Rt=0.62 min; [M+H]⁺=702.

¹H NMR (400 MHz, DMSO-d₆) δ 10.29 (s, 1H), 7.72 (d, J=2.4 Hz, 1H), 7.52 (d, J=2.4 Hz, 1H), 7.25-7.18 (m, 2H), 7.04 (s, 1H), 6.91 (d, J=8.7 Hz, 2H), 6.88 (s, 1H), 4.80 (s, 2H), 3.84 (m, 1H), 3.76 (s, 3H), 3.71 (m, 7H), 3.49 (s, 3H), 3.45 (m, 3H), 3.15 (m, 2H), 2.70 (m, 4H), 2.12 (m, 2H), 2.04 (s, 3H), 1.80 (m, 4H), 1.45 (m, 3H), 1.36-1.24 (m, 1H).

### Compound A8: 1-(4-(2-(4-((1-(4-(1,5-Dimethyl-6-oxo-1,6-dihydropyridin-3-yl)-2,6-dimethoxybenzyl)piperidin-4-yl)oxy)piperidin-1-yl)-2-oxoethoxy)phenyl)dihydropyrimidine-2,4(1H,3H)-dione

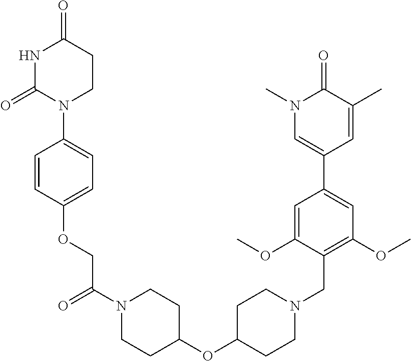 [SMILES: COc1cc(-c2cc(C)c(=O)n(C)c2)cc(OC)c1CN1CCC(OC2CCN(C(=O)COc3ccc(N4CCC(=O)NC4=O)cc3)CC2)CC1]

To a 10 ml round bottom flask were added 1-(4-(2-oxo-2-(4-(piperidin-4-yloxy)piperidin-1-yl)ethoxy)phenyl)dihydropyrimidine-2,4(1H,3H)-dione hydrochloride (intermediate 30, 186 mg, 0.339 mmol), TEA (0.150 ml, 1.076 mmol), 4-(1,5-dimethyl-6-oxo-1,6-dihydropyridin-3-yl)-2,6-dimethoxybenzaldehyde (intermediate 9, 97 mg, 0.339 mmol), a solution of ZnCl₂ (0.5 M) in THE (0.700 ml, 0.350 mmol) and MeOH (1.5 ml). The RM was stirred at RT for 7 h, solid NaBH₃CN (22 mg, 0.350 mmol) was added and the RM was stirred at RT overnight. The mixture was concentrated and the residue purified by reversed phase chromatography on a REDISEP® Gold HP C18 column (15.5 g) eluting with ACN (from 2% to 90%) in an aq. solution of NH₄HCO₃ (0.1%), followed by a purification using SFC on a Reprospher PEI column (250×30 mm, 100 Å, 5 m) eluting with MeOH (from 30% to 40%) in CO₂, yielding the title compound as a solid (80 mg).

Method L: Rt=2.89 min; [M+H]⁺=702.

¹H NMR (400 MHz, DMSO-d₆) δ 10.31 (s, 1H), 8.04 (s, 1H), 7.81 (s, 1H), 7.23 (m, 2H), 6.88 (m, 4H), 4.81 (s, 2H), 4.08-2.91 (m, 19H), 2.70 (m, 4H), 2.11 (m, 5H), 1.93-1.65 (m, 4H), 1.35 (m, 4H).

### Compound A9: 1-(2-Chloro-5-(4-((4-(2,6-dimethoxy-4-(2-methyl-1-oxo-1,2-dihydro-2,7-naphthyridin-4-yl)phenoxy)piperidin-1-yl)methyl)piperidine-1-carbonyl)phenyl)dihydropyrimidine-2,4(1H,3H)-dione

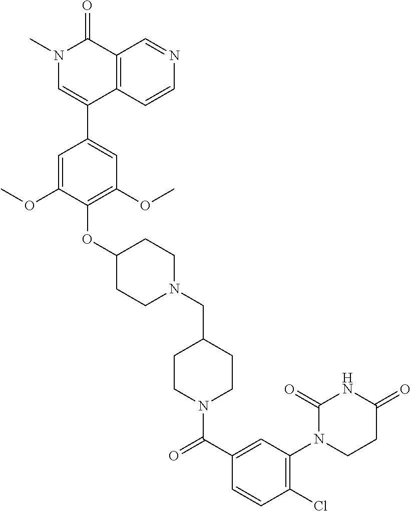 [SMILES: COc1cc(-c2cn(C)c(=O)c3cnccc23)cc(OC)c1OC1CCN(CC2CCN(C(=O)c3ccc(Cl)c(N4CCC(=O)NC4=O)c3)CC2)CC1]

To a 5 ml round bottom flask were added HATU (42 mg, 0.110 mmol), 4-chloro-3-(2,4-dioxotetrahydropyrimidin-1(2H)-yl)benzoic acid (intermediate 24, 27 mg, 0.101 mmol), DIPEA (0.050 ml, 0.286 mmol) and DMF (1 ml). The RM was stirred at RT for 30 min, a solution of 4-(3,5-dimethoxy-4-((1-(piperidin-4-ylmethyl)piperidin-4-yl)oxy)phenyl)-2-methyl-2,7-naphthyridin-1(2H)-one TFA salt (intermediate 21, 68 mg, 0.092 mmol), DIPEA (0.050 ml, 0.286 mmol) and DMF (0.5 ml) was added and the RM was stirred at RT for 2 days. Solid HATU (42 mg, 0.110 mmol) and 4-chloro-3-(2,4-dioxotetrahydropyrimidin-1(2H)-yl)benzoic acid (intermediate 24, 27 mg, 0.101 mmol) were added and the RM was stirred at RT for 3 h. The mixture was directly purified by reversed phase chromatography on a REDISEP® Gold HP C18 column (15.5 g) eluting with ACN (from 2% to 100%) in an aq. solution of NH₄HCO₃ (0.1%), yielding the title compound as a solid (38 mg).

Method L: Rt=2.90 min; [M+H]⁺=744.

¹H NMR (400 MHz, DMSO-d₆) δ 10.51 (s, 1H), 9.45-9.41 (m, 1H), 8.72 (d, J=5.7 Hz, 1H), 7.83 (s, 1H), 7.64 (d, J=8.1 Hz, 1H), 7.56-7.51 (m, 2H), 7.39 (dd, J=8.2, 2.0 Hz, 1H), 6.75 (m, 2H), 4.45 (s, 1H), 4.20-3.96 (m, 1H), 3.69 (m, 13H), 3.06 (m, 2H), 2.91-2.67 (m, 4H), 1.82 (m, 10H), 1.11 (m, 2H).

### Compound A10: 1-(2-Chloro-5-(4-((4-(2-methoxy-4-(2-methyl-1-oxo-1,2-dihydro-2,7-naphthyridin-4-yl)phenoxy)piperidin-1-yl)methyl)piperidine-1-carbonyl)phenyl)dihydropyrimidine-2,4(1H,3H)-dione

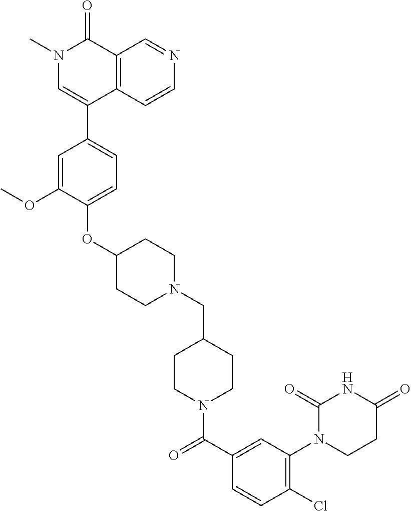 [SMILES: COc1cc(-c2cn(C)c(=O)c3cnccc23)ccc1OC1CCN(CC2CCN(C(=O)c3ccc(Cl)c(N4CCC(=O)NC4=O)c3)CC2)CC1]

To a 5 ml round bottom flask were added HATU (21 mg, 0.055 mmol), 4-chloro-3-(2,4-dioxotetrahydropyrimidin-1(2H)-yl)benzoic acid (intermediate 24, 14 mg, 0.052 mmol), DIPEA (0.050 ml, 0.286 mmol) and DMF (0.5 ml). The RM was stirred at RT for 30 min, a solution of 4-(3-methoxy-4-((1-(piperidin-4-ylmethyl)piperidin-4-yl)oxy)phenyl)-2-methyl-2,7-naphthyridin-1(2H)-one TFA salt (intermediate 20, 32 mg, 0.046 mmol), DIPEA (0.050 ml, 0.286 mmol) and DMF (0.5 ml) was added and the RM was stirred at RT for 2 days. The mixture was directly purified by reversed phase chromatography on a REDISEP® Gold HP C18 column (15.5 g) eluting with ACN (from 2% to 100%) in an aq. solution of NH₄HCO₃ (0.1%), yielding the title compound as a solid (22 mg).

Method L: Rt=2.60 min; [M+H]⁺=713.

¹H NMR (400 MHz, DMSO-d₆) δ 10.51 (s, 1H), 9.46 (s, 1H), 8.70 (d, J=5.7 Hz, 1H), 7.78 (s, 1H), 7.64 (d, J=8.2 Hz, 1H), 7.55 (d, J=2.0 Hz, 1H), 7.48 (d, J=5.7 Hz, 1H), 7.39 (dd, J=8.2, 2.0 Hz, 1H), 7.12 (d, J=8.3 Hz, 1H), 7.05 (d, J=2.0 Hz, 1H), 6.94 (dd, J=8.2, 2.0 Hz, 1H), 4.39 (m, 2H), 3.80 (s, 3H), 3.78-3.60 (m, 3H), 3.58 (s, 3H), 3.14-2.77 (m, 2H), 2.70 (m, 4H), 2.27-1.56 (m, 11H), 1.09 (m, 2H).

### Compound A11: 1-(3-(4-((4-(2-Methoxy-4-(2-methyl-1-oxo-1,2-dihydro-2,7-naphthyridin-4-yl)phenoxy)piperidin-1-yl)methyl)piperidine-1-carbonyl)phenyl)dihydropyrimidine-2,4(1H,3H)-dione

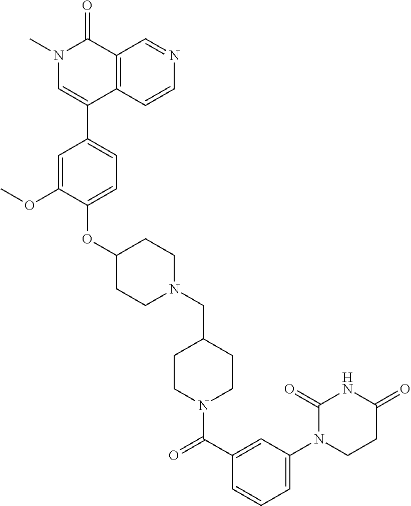 [SMILES: COc1cc(-c2cn(C)c(=O)c3cnccc23)ccc1OC1CCN(CC2CCN(C(=O)c3cccc(N4CCC(=O)NC4=O)c3)CC2)CC1]

To a 5 ml round bottom flask were added HATU (21 mg, 0.055 mmol), 3-(2,4-dioxotetrahydropyrimidin-1(2H)-yl)benzoic acid (intermediate 22, 12 mg, 0.051 mmol), DIPEA (0.050 ml, 0.286 mmol) and DMF (0.5 ml). The mixture was stirred at RT for 30 min, a solution of 4-(3-methoxy-4-((1-(piperidin-4-ylmethyl)piperidin-4-yl)oxy)phenyl)-2-methyl-2,7-naphthyridin-1(2H)-one TFA salt (intermediate 20, 32 mg, 0.046 mmol), DIPEA (0.050 ml, 0.286 mmol) and DMF (0.5 ml) was added and the RM was stirred at RT for 2 days. The mixture was directly purified by reversed phase chromatography on a REDISEP® Gold HP C18 column (15.5 g) eluting with ACN (from 2% to 100%) in an aq. solution of NH₄HCO₃ (0.1%), followed by a purification using SFC on a Reprospher PEI column (250×30 mm, 100 Å, 5 m) eluting with MeOH (from 24% to 34%) in CO₂, yielding the title compound as a solid (23.1 mg).

Method D: Rt=0.57 min; [M+H]⁺=679.

¹H NMR (600 MHz, DMSO-d₆) δ 10.42 (s, 1H), 9.43 (s, 1H), 8.70 (d, J=5.6 Hz, 1H), 7.78 (s, 1H), 7.48 (d, J=5.7 Hz, 1H), 7.45 (t, J=7.7 Hz, 1H), 7.39 (d, J=7.7 Hz, 1H), 7.35 (t, J=1.9 Hz, 1H), 7.21 (dt, J=7.7 Hz, 1H), 7.11 (d, J=8.2 Hz, 1H), 7.04 (d, J=2.0 Hz, 1H), 6.94 (dd, J=8.2, 2.0 Hz, 1H), 4.41 to 4.52 (s, 1H), 4.33 (m, 1H), 3.82 (m, 2H), 3.80 (s, 3H), 3.58 to 3.66 (s, 1H), 3.58 (s, 3H), 2.99 to 3.1 (m, 1H), 2.76 (m, 1H), 2.72 (m, 4H), 2.11 to 2.23 (m, 4H), 1.94 (m, 2H), 1.74 to 1.86 (m, 2H), 1.60 to 1.72 (m, 3H), 0.97 to 1.14 (m, 2H).

### Compound A12: 1-(2-Methoxy-5-(4-((4-(4-(2-methyl-1-oxo-1,2-dihydro-2,7-naphthyridin-4-yl)phenoxy)piperidin-1-yl)methyl)piperidine-1-carbonyl)phenyl)dihydropyrimidine-2,4(1H,3H)-dione

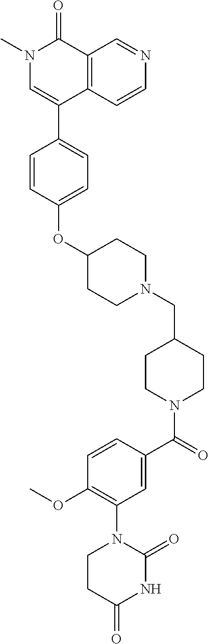 [SMILES: COc1ccc(C(=O)N2CCC(CN3CCC(Oc4ccc(-c5cn(C)c(=O)c6cnccc56)cc4)CC3)CC2)cc1N1CCC(=O)NC1=O]

### Step 1: tert-butyl 4-(4-(4,4,5,5-tetramethyl-1,3,2-dioxaborolan-2-yl)phenoxy)piperidine-1-carboxylate

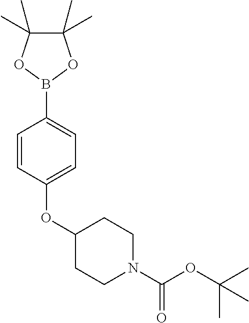 [SMILES: CC(C)(C)OC(=O)N1CCC(Oc2ccc(B3OC(C)(C)C(C)(C)O3)cc2)CC1]

To a 100 ml round bottom flask were added under an argon atmosphere 4-hydroxyphenylboronic acid pinacol ester (257 mg, 1.168 mmol), tert-butyl 4-hydroxypiperidine-1-carboxylate (264 mg, 1.285 mmol), PPh₃ (337 mg, 1.285 mmol) and THE (5 ml). DIAD (0.250 ml, 1.285 mmol) was added dropwise and the RM was stirred at RT overnight. The RM was poured into a mixture of EtOAc and an aq. sat. solution of Na₂CO₃, the aq. phase was extracted with EtOAc, the combined organic phases were washed with brine, dried over MgSO₄ and the residue was purified by chromatography on silica gel eluting with EtOAc (from 0% to 20%) in CHX, yielding the title compound as a solid (264 mg).

Method D: Rt=1.49 min; [M-tBu+H]⁺=348.

### Step 2: tert-butyl 4-(4-(2-methyl-1-oxo-1,2-dihydro-2,7-naphthyridin-4-yl)phenoxy)piperidine-1-carboxylate

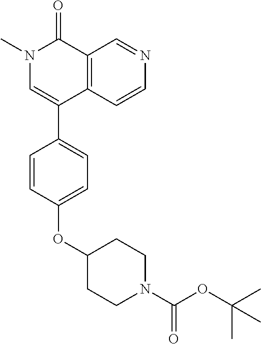 [SMILES: Cn1cc(-c2ccc(OC3CCN(C(=O)OC(C)(C)C)CC3)cc2)c2ccncc2c1=O]

To a 25 ml vial were added under an argon atmosphere tert-butyl 4-(4-(4,4,5,5-tetramethyl-1,3,2-dioxaborolan-2-yl)phenoxy)piperidine-1-carboxylate (264 mg, 0.511 mmol), 4-bromo-2-methyl-2,7-naphthyridin-1(2H)-one (intermediate 5, 123 mg, 0.511 mmol), K₂CO₃ (212 mg, 1.532 mmol), ACN (4 ml) and water (1 ml). Solid PdCl₂(dppf) (37 mg, 0.051 mmol) was added and the RM was stirred at 100° C. for 1.5 h. The mixture was cooled to RT, filtered through CELITE®, the filtrate was concentrated and the residue purified by reversed phase chromatography on a REDISEP® Gold HP C18 column (15.5 g) eluting with ACN (from 2% to 100%) in an aq. solution of TFA (0.1%), yielding the title compound as a solid (112 mg).

Method D: Rt=0.49 min; [M+H]⁺=336.

### Step 3: 2-methyl-4-(4-(piperidin-4-yloxy)phenyl)-2,7-naphthyridin-1(2H)-one

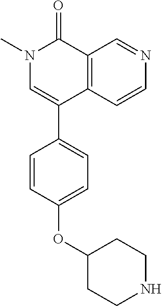 [SMILES: Cn1cc(-c2ccc(OC3CCNCC3)cc2)c2ccncc2c1=O]

To a 10 ml round bottom flask were added tert-butyl 4-(4-(2-methyl-1-oxo-1,2-dihydro-2,7-naphthyridin-4-yl) phenoxy)piperidine-1-carboxylate (144 mg, 0.331 mmol), TEA (0.750 ml, 9.73 mmol) and DCM (4 ml). The RM was stirred at RT for 1 h, concentrated and the residue was purified by reversed phase chromatography on a REDISEP® Gold HP C18 column (15.5 g) eluting with ACN (from 2% to 100%) in an aq. solution of TFA (0.1%), yielding the corresponding TFA salt of the title compound as a solid (144 mg).

Method D: Rt=1.16 min; [M+H]=436.

### Step 4: tert-butvl 4-((4-(4-(2-methyl-1-oxo-1,2-dihydro-2,7-naphthyridin-4-yl) phenoxy)piperidin-1-yl)methyl)piperidine-1-carboxylate

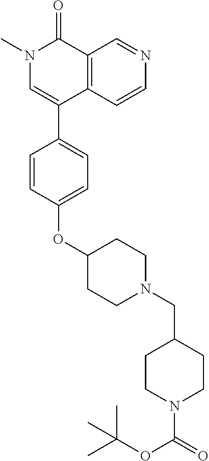 [SMILES: Cn1cc(-c2ccc(OC3CCN(CC4CCN(C(=O)OC(C)(C)C)CC4)CC3)cc2)c2ccncc2c1=O]

To a 10 ml round bottom flask were added 2-methyl-4-(4-(piperidin-4-yloxy)phenyl)-2,7-naphthyridin-1(2H)-one TFA salt (112 mg, 0.247 mmol), tert-butyl 4-formylpiperidine-1-carboxylate (63 mg, 0.295 mmol), TEA (0.100 ml, 0.717 mmol), a solution of ZnCl₂ (0.5 M) in THF (0.550 ml, 0.275 mmol) and MeOH (2 ml). The RM was stirred at RT for 7 h, solid NaBH₃CN (17 mg, 0.271 mmol) was added and the RM was stirred at RT for 20 h. The mixture was concentrated and the residue was directly used for the next step without further purification.

Method D: Rt=0.76 min; [M+H]⁺=533.

### Step 5: 2-methyl-4-(4-((1-(piperidin-4-ylmethyl)piperidin-4-yl)oxy)phenyl)-2,7-naphthyridin-1(2H)-one

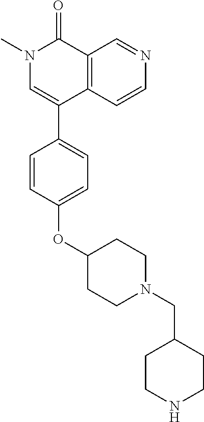 [SMILES: Cn1cc(-c2ccc(OC3CCN(CC4CCNCC4)CC3)cc2)c2ccncc2c1=O]

To a 25 ml round bottom flask were added tert-butyl 4-((4-(4-(2-methyl-1-oxo-1,2-dihydro-2,7-naphthyridin-4-yl)phenoxy)piperidin-1-yl)methyl)piperidine-1-carboxylate (0.247 mmol), TFA (0.250 ml, 3.24 mmol) and DCM (2 ml). The RM was stirred at RT for 1 h, concentrated and the residue was purified by reversed phase chromatography on a REDISEP® Gold HP C18 column (15.5 g) eluting with ACN (from 2% to 100%) in an aq. solution of TFA (0.1%), yielding the corresponding TFA salt of the title compound as a solid (172 mg).

Method D: Rt=0.43 min; [M+H]⁺=433.

### Step 6: 1-(2-methoxy-5-(4-((4-(4-(2-methyl-1-oxo-1,2-dihydro-2,7-naphthyridin-4-yl)phenoxy)piperidin-1-yl)methyl)piperidine-1-carbonyl)phenyl)dihydropyrimidine-2,4(1H,3H)-dione

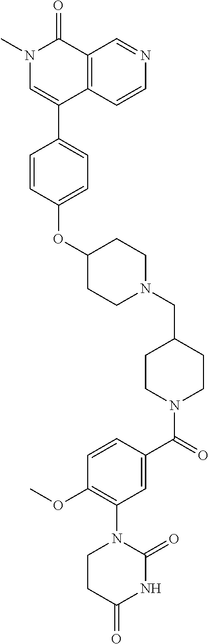 [SMILES: COc1ccc(C(=O)N2CCC(CN3CCC(Oc4ccc(-c5cn(C)c(=O)c6cnccc56)cc4)CC3)CC2)cc1N1CCC(=O)NC1=O]

To a 50 ml round bottom flask were added HATU (60 mg, 0.158 mmol), 3-(2,4-dioxotetrahydropyrimidin-1(2H)-yl)-4-methoxybenzoic acid (intermediate 25, 38 mg, 0.144 mmol), DIPEA (0.100 ml, 0.573 mmol) and DMF (1.5 ml). The mixture was stirred at RT for 30 min, a solution of 2-methyl-4-(4-((1-(piperidin-4-ylmethyl)piperidin-4-yl)oxy)phenyl)-2,7-naphthyridin-1(2H)-one TFA salt (90 mg, 0.129 mmol), DIPEA (0.100 ml, 0.573 mmol) and DMF (0.5 ml) was added and the RM was stirred at RT for 20 h. The mixture was directly purified by reversed phase chromatography on a REDISEP® Gold HP C18 column (15.5 g) eluting with ACN (from 2% to 100%) in an aq. solution of NH₄HCO₃ (0.1%), yielding the title compound as a solid (70 mg).

Method L: Rt=2.48 min; [M−H]⁺=677.

¹H NMR (400 MHz, DMSO-d₆) δ 10.33 (s, 1H), 9.43 (s, 1H), 8.70 (d, J=5.7 Hz, 1H), 7.75 (s, 1H), 7.44-7.27 (m, 5H), 7.15 (d, J=8.5 Hz, 1H), 7.08 (m, 2H), 4.42 (m, 2H), 3.84 (s, 3H), 3.58 (m, 5H), 2.84 (m, 2H), 2.75-2.65 (m, 4H), 2.19 (m, 4H), 2.02-0.98 (m, 10H).

### Compound A13: 1-(2-Methoxy-5-(4-((4-(2-methoxy-4-(1,4,5-trimethyl-6-oxo-1,6-dihydropyridin-3-yl)benzyl)piperazin-1-yl)methyl)piperidine-1-carbonyl)phenyl)dihydropyrimidine-2,4(1H,3H)-dione

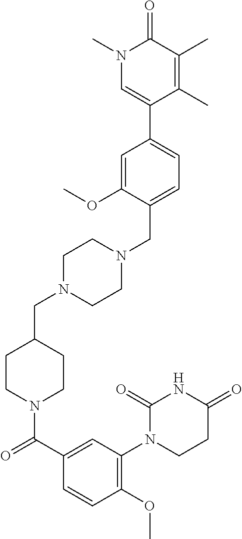 [SMILES: COc1cc(-c2cn(C)c(=O)c(C)c2C)ccc1CN1CCN(CC2CCN(C(=O)c3ccc(OC)c(N4CCC(=O)NC4=O)c3)CC2)CC1]

To a 50 ml round bottom flask were added HATU (58 mg, 0.153 mmol), 3-(2,4-dioxotetrahydropyrimidin-1(2H)-yl)-4-methoxybenzoic acid (intermediate 25, 37 mg, 0.140 mmol), DIPEA (0.100 ml, 0.573 mmol) and DMF (1 ml). The RM was stirred at RT for 30 min, a solution of 5-(3-methoxy-4-((4-(piperidin-4-ylmethyl)piperazin-1-yl)methyl)phenyl)-1,3,4-trimethylpyridin-2(1H)-one TFA salt (intermediate 19, 100 mg, 0.126 mmol), DIPEA (0.100 ml, 0.573 mmol) and DMF (0.5 ml) was added and the RM was stirred at RT overnight. The mixture was directly purified by reversed phase chromatography on a REDISEP® Gold HP C18 column (15.5 g) eluting with ACN (from 2% to 100%) in an aq. solution of NH₄HCO₃ (0.1%) yielding the title compound as a solid (72 mg).

Method L: Rt=2.62 min; [M+H]⁺=685.

¹H NMR (400 MHz, DMSO-d₆) δ 10.33 (s, 1H), 7.48 (s, 1H), 7.35 (dd, J=8.4, 2.1 Hz, 1H), 7.31 (m, 2H), 7.15 (d, J=8.5 Hz, 1H), 6.85 (d, J=1.6 Hz, 1H), 6.82 (dd, J=7.6, 1.6 Hz, 1H), 4.35 (m, 2H), 3.84 (s, 3H), 3.78 (s, 3H), 3.59 (t, J=6.7 Hz, 2H), 3.45 (m, 5H), 3.10-2.72 (m, 2H), 2.68 (t, J=6.7 Hz, 2H), 2.46-2.25 (m, 8H), 2.13 (d, J=7.0 Hz, 2H), 2.04 (m, 6H), 1.72 (m, 3H), 1.05 (m, 2H).

### Compound A14: 1-(2-Chloro-5-(4-((4-(2-methoxy-4-(1,4,5-trimethyl-6-oxo-1,6-dihydropyridin-3-yl)benzyl)piperazin-1-yl)methyl)piperidine-1-carbonyl)phenyl)dihydropyrimidine-2,4(1H,3H)-dione

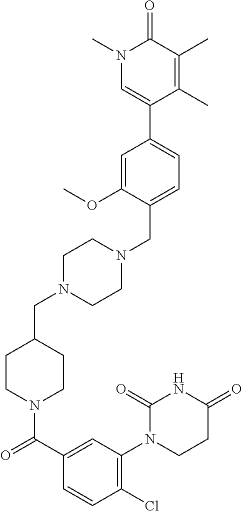 [SMILES: COc1cc(-c2cn(C)c(=O)c(C)c2C)ccc1CN1CCN(CC2CCN(C(=O)c3ccc(Cl)c(N4CCC(=O)NC4=O)c3)CC2)CC1]

To a 50 ml round bottom flask were added HATU (58 mg, 0.153 mmol), 4-chloro-3-(2,4-dioxotetrahydropyrimidin-1(2H)-yl)benzoic acid (intermediate 24, 38 mg, 0.141 mmol), DIPEA (0.100 ml, 0.573 mmol) and DMF (1 ml). The RM was stirred at RT for 30 min, a solution of 5-(3-methoxy-4-((4-(piperidin-4-ylmethyl)piperazin-1-yl)methyl)phenyl)-1,3,4-trimethylpyridin-2(1H)-one TFA salt (intermediate 19, 135 mg, 0.175 mmol), DIPEA (0.100 ml, 0.573 mmol) and DMF (0.5 ml) was added and the RM was stirred at RT overnight. The mixture was directly purified by reversed phase chromatography on a REDISEP® Gold HP C18 column (15.5 g) eluting with ACN (from 2% to 100%) in an aq. solution of NH₄HCO₃ (0.1%), yielding the title compound as a solid (61 mg).

Method L: Rt=2.79 min; [M+H]⁺=689.

¹H NMR (400 MHz, DMSO-d₆) δ 10.50 (s, 1H), 7.63 (d, J=8.2 Hz, 1H), 7.57-7.51 (m, 1H), 7.47 (s, 1H), 7.42-7.34 (m, 1H), 7.30 (d, J=7.6 Hz, 1H), 6.85 (s, 1H), 6.82 (d, J=7.9 Hz, 1H), 4.77 (m, 1H), 3.78 (s, 3H), 3.73 (m, 7H), 3.10-2.66 (m, 5H), 2.41 (m, 8H), 2.14 (m, 2H), 2.03 (m, 6H), 1.71 (m, 3H), 1.06 (m, 2H).

### Compound A15: 1-(3-(4-((4-(2-Methoxy-4-(1,4,5-trimethyl-6-oxo-1,6-dihydropyridin-3-yl)benzyl)piperazin-1-yl)methyl)piperidine-1-carbonyl)phenyl)dihydropyrimidine-2,4(1H,3H)-dione

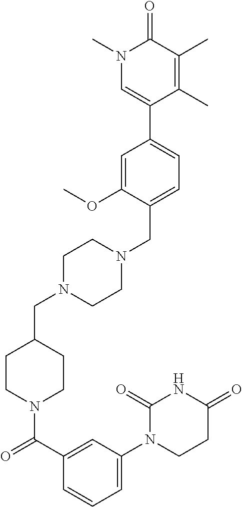 [SMILES: COc1cc(-c2cn(C)c(=O)c(C)c2C)ccc1CN1CCN(CC2CCN(C(=O)c3cccc(N4CCC(=O)NC4=O)c3)CC2)CC1]

To a 50 ml round bottom flask were added HATU (58 mg, 0.153 mmol), 3-(2,4-dioxotetrahydropyrimidin-1(2H)-yl)benzoic acid (intermediate 22, 33 mg, 0.141 mmol), DIPEA (0.100 ml, 0.573 mmol) and DMF (1 ml). The RM was stirred at RT for 30 min, a solution of 5-(3-methoxy-4-((4-(piperidin-4-ylmethyl)piperazin-1-yl)methyl)phenyl)-1,3,4-trimethylpyridin-2(1H)-one TFA salt (intermediate 19, 100 mg, 0.126 mmol), DIPEA (0.100 ml, 0.573 mmol) and DMF (0.5 ml) was added and the RM was stirred at RT overnight. The mixture was directly purified by reversed phase chromatography on a REDISEP® Gold HP C18 column (15.5 g) eluting with ACN (from 2% to 100%) in an aq. solution of NH₄HCO₃ (0.1%), yielding the title compound as a solid (57 mg).

Method L: Rt=2.60 min; [M+H]⁺=655.

¹H NMR (400 MHz, DMSO-d₆) δ 10.40 (s, 1H), 7.50-7.36 (m, 3H), 7.36-7.28 (m, 2H), 7.21 (d, J=7.4 Hz, 1H), 6.88-6.79 (m, 2H), 4.44 (m, 1H), 3.80 (m, 5H), 3.69-3.53 (m, 1H), 3.45 (m, 5H), 3.01 (m, 2H), 2.71 (t, J=6.6 Hz, 2H), 2.40 (m, 8H), 2.14 (m, 2H), 2.04 (m, 6H), 1.73 (m, 3H), 1.06 (m, 2H).

### Compound A16: 1-(3-(4-((1-(4-(1,5-Dimethyl-6-oxo-1,6-dihydropyridin-3-yl)-2,5-dimethoxybenzyl)piperidin-4-yl)oxy)piperidine-1-carbonyl)phenyl)dihydropyrimidine-2,4(1H,3H)-dione

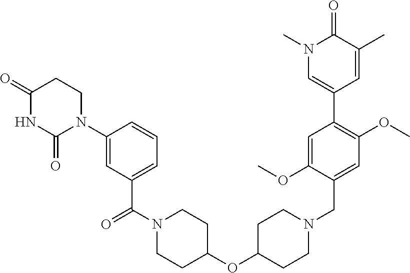 [SMILES: COc1cc(-c2cc(C)c(=O)n(C)c2)c(OC)cc1CN1CCC(OC2CCN(C(=O)c3cccc(N4CCC(=O)NC4=O)c3)CC2)CC1]

### Step 1: tert-butyl 4-((1-(3-(2,4-dioxotetrahydropyrimidin-1(2H)-yl)benzoyl)piperidin-4-yl)oxy)piperidine-1-carboxylate

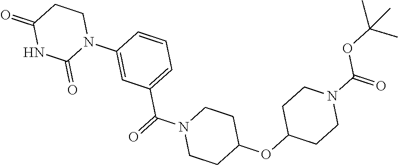 [SMILES: CC(C)(C)OC(=O)N1CCC(OC2CCN(C(=O)c3cccc(N4CCC(=O)NC4=O)c3)CC2)CC1]

To a 25 ml round bottom flask were added HATU (401 mg, 1.055 mmol), 3-(2,4-dioxotetrahydropyrimidin-1(2H)-yl)benzoic acid (intermediate 22, 206 mg, 0.879 mmol), DIPEA (0.500 ml, 2.86 mmol) and DMF (5 ml). The RM was stirred at RT for 30 min, solid tert-butyl 4-(piperidin-4-yloxy)piperidine-1-carboxylate (intermediate 1, 250 mg, 0.879 mmol) was added and the RM was stirred at RT overnight. The mixture was directly purified by reversed phase chromatography on a REDISEP® Gold HP C18 column (50 g) eluting with ACN (from 2% to 100%) in an aq. solution of TFA (0.1%), yielding the title compound as a solid (361 mg).

Method D: Rt=0.91 min; [M+H]⁺=501.

### Step 2:1-(3-(4-(piperidin-4-yloxy)piperidine-1-carbonyl)phenyl)dihydropyrimidine-2,4(1H,3H)-dione

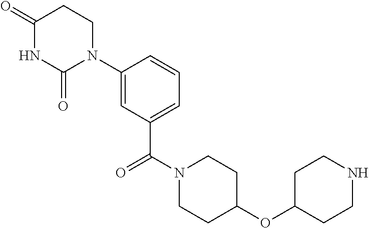 [SMILES: O=C1CCN(c2cccc(C(=O)N3CCC(OC4CCNCC4)CC3)c2)C(=O)N1]

To a 25 ml round bottom flask were added tert-butyl 4-((1-(3-(2,4-dioxotetrahydropyrimidin-1(2H)-yl)benzoyl)piperidin-4-yl)oxy)piperidine-1-carboxylate (361 mg, 0.721 mmol), a solution of HCl (4 M) in 1,4-dioxane (4 ml), MeOH (2 ml) and 1,4-dioxane (4 ml). The RM was stirred at RT for 3 h, concentrated, the residue was dissolved in a mixture of water and ACN and freeze dried, yielding the corresponding hydrochloride salt of the title compound as a solid (340 mg).

Method D: Rt=0.39 min; [M+H]⁺=401.

### Step 3:1-(3-(4-((1-(4-(1,5-dimethyl-6-oxo-1,6-dihydropyridin-3-yl)-2,5-dimethoxybenzyl)piperidin-4-yl)oxy)piperidine-1-carbonyl)phenyl)dihydropyrimidine-2,4(1H,3H)-dione

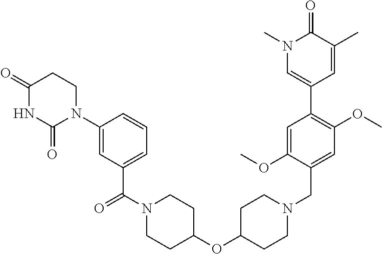 [SMILES: COc1cc(-c2cc(C)c(=O)n(C)c2)c(OC)cc1CN1CCC(OC2CCN(C(=O)c3cccc(N4CCC(=O)NC4=O)c3)CC2)CC1]

To a 10 ml round bottom flask were added 1-(3-(4-(piperidin-4-yloxy)piperidine-1-carbonyl)phenyl)dihydropyrimidine-2,4(1H,3H)-dione hydrochloride (122 mg, 0.260 mmol), HOAc (0.013 ml, 0.225 mmol), NaOAc (27.6 mg, 0.337 mmol), MeOH (1 ml) and DCM (1 ml). The RM was stirred at 0° C. for 10 min, solid 4-(1,5-dimethyl-6-oxo-1,6-dihydropyridin-3-yl)-2,5-dimethoxybenzaldehyde (intermediate 8, 70 mg, 0.236 mmol) was added and the RM was stirred at RT for 30 min. Solid NaBH(OAc)₃ (100 mg, 0.473 mmol) was added and the RM was stirred at RT overnight. MeOH (2 ml) was added, the RM was stirred at RT for 2 h, the RM was concentrated, DIPEA (0.150 ml, 0.859 mmol), a solution of ZnCl₂ (0.5 M) in THE (0.500 ml, 0.250 mmol) and MeOH (2 ml) were added and the RM was stirred at RT overnight. Solid NaBH₃CN (15 mg, 0.239 mmol) was added and the RM was stirred at RT overnight. The RM was filtered over CELITE®, the solids were washed with MeOH and the combined filtrates evaporated. The residue was purified by reversed phase chromatography on a REDISEP® Gold HP C18 column (15.5 g) eluting with ACN (from 2% to 100%) in an aq. solution of NH₄HCO₃ (0.1%), followed by a purification using SFC on a Reprospher PEI column (250×30 mm, 100 Å, 5 m) eluting with MeOH (from 25% to 35%) in CO₂, yielding the title compound as a solid (56 mg).

Method L: Rt=2.61 min; [M+H]⁺=672.

¹H NMR (400 MHz, DMSO-d₆) δ 10.40 (s, 1H), 7.71 (d, J=2.5 Hz, 1H), 7.52 (dd, J=2.6, 1.3 Hz, 1H), 7.47-7.38 (m, 2H), 7.36 (d, J=1.8 Hz, 1H), 7.23 (dt, J=7.4, 1.5 Hz, 1H), 7.04 (s, 1H), 6.88 (s, 1H), 4.05 (m, 4H), 3.82 (t, J=6.6 Hz, 2H), 3.75 (s, 3H), 3.71 (m, 4H), 3.47 (m, 6H), 2.71 (m, 4H), 2.12 (m, 2H), 2.04 (s, 3H), 1.90-1.30 (m, 8H).

### Compound A17: 1-(5-(4-((4-(2-Chloro-4-(2-methyl-1-oxo-1,2-dihydro-2,7-naphthyridin-4-yl)phenoxy)piperidin-1-yl)methyl)piperidine-1-carbonyl)-2-methoxyphenyl)dihydropyrimidine-2,4(1H,3H)-dione

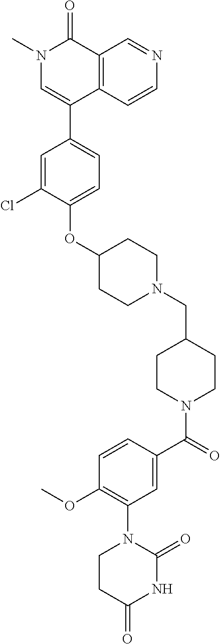 [SMILES: COc1ccc(C(=O)N2CCC(CN3CCC(Oc4ccc(-c5cn(C)c(=O)c6cnccc56)cc4Cl)CC3)CC2)cc1N1CCC(=O)NC1=O]

### Step 1: 4-(3-chloro-4-hydroxyphenyl)-2-methyl-2,7-naphthyridin-1(2H)-one

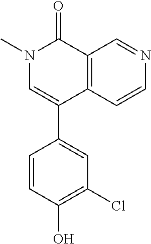 [SMILES: Cn1cc(-c2ccc(O)c(Cl)c2)c2ccncc2c1=O]

To a 50 ml round bottom flask were added under an argon atmosphere 4-bromo-2-methyl-2,7-naphthyridin-1(2H)-one (intermediate 5, 400 mg, 1.673 mmol), (3-chloro-4-hydroxyphenyl)boronic acid (332 mg, 1.924 mmol), an aq. solution of NaOAc (2 M, 2.510 ml, 5.02 mmol) and DMF (10 ml). Solid PdCl₂(dppf)-CH₂Cl₂ (124 mg, 0.167 mmol) was added and the RM was stirred at 100° C. for 1 h. The mixture was cooled to RT, filtered through CELITE®, the combined filtrates were concentrated and purified by reversed phase chromatography on a REDISEP® Gold HP C18 column (15.5 g) eluting with ACN (from 2% to 100%) in an aq. solution of TFA (0.1%), yielding the title compound as a solid (316 mg).

Method D: Rt=0.70 min; [M+H]⁺=287.

### Step 2: tert-butyl 4-(2-chloro-4-(2-methyl-1-oxo-1,2-dihydro-2,7-naphthyridin-4-yl)phenoxy)piperidine-1-carboxylate

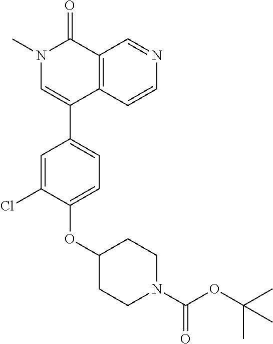 [SMILES: Cn1cc(-c2ccc(OC3CCN(C(=O)OC(C)(C)C)CC3)c(Cl)c2)c2ccncc2c1=O]

To a 100 ml round bottom flask were added under an argon atmosphere 4-(3-chloro-4-hydroxyphenyl)-2-methyl-2,7-naphthyridin-1(2H)-one (316 mg, 1.102 mmol), tert-butyl 4-hydroxypiperidine-1-carboxylate (212 mg, 1.032 mmol), PPh₃ (270 mg, 1.029 mmol) and toluene (7 ml). DIAD (0.200 ml, 1.029 mmol) was added dropwise and the RM was stirred at RT overnight. Tert-butyl 4-hydroxypiperidine-1-carboxylate (212 mg, 1.032 mmol), PPh₃ (270 mg, 1.029 mmol) and DIAD (0.200 ml, 1.029 mmol) were added and the RM was stirred at RT for 2 h. The RM was poured into a mixture of EtOAc and an aq. sat. solution of NaHCO₃, the aq. phase was extracted with EtOAc, the combined organic phases were washed with brine, dried over MgSO₄, concentrated and the residue was purified by chromatography on silica gel eluting with EtOAc (from 0% to 100%) in CHX, yielding the title compound as a solid (286 mg).

Method D: Rt=1.18 min; [M+H]⁺=470.

### Step 3: 4-(3-chloro-4-(piperidin-4-yloxy)phenyl)-2-methyl-2,7-naphthyridin-1(2H)-one

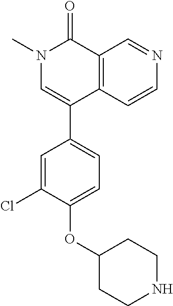 [SMILES: Cn1cc(-c2ccc(OC3CCNCC3)c(Cl)c2)c2ccncc2c1=O]

To a 25 ml round bottom flask were added tert-butyl 4-(2-chloro-4-(2-methyl-1-oxo-1,2-dihydro-2,7-naphthyridin-4-yl)phenoxy)piperidine-1-carboxylate (286 mg, 0.609 mmol), TFA (1 ml, 12.98 mmol) and DCM (4 ml). The RM was stirred at RT for 1 h and concentrated to dryness, yielding the corresponding TFA salt of the title compound as a solid (291 mg).

Method L: Rt=2.12 min; [M+H]⁺=370.

### Step 4: tert-butyl 4-((4-(2-chloro-4-(2-methyl-1-oxo-1,2-dihydro-2,7-naphthyridin-4-yl)phenoxy)piperidin-1-yl)methyl)piperidine-1-carboxylate

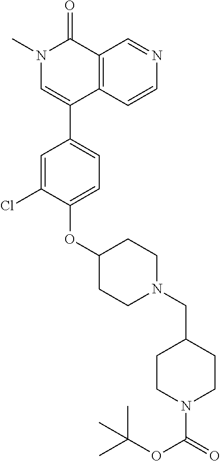 [SMILES: Cn1cc(-c2ccc(OC3CCN(CC4CCN(C(=O)OC(C)(C)C)CC4)CC3)c(Cl)c2)c2ccncc2c1=O]

To a 10 ml round bottom flask were added 4-(3-chloro-4-(piperidin-4-yloxy)phenyl)-2-methyl-2,7-naphthyridin-1(2H)-one TFA salt (280 mg, 0.579 mmol), tert-butyl 4-formylpiperidine-1-carboxylate (148 mg, 0.694 mmol), TEA (0.250 ml, 1.794 mmol), a solution of ZnCl₂ (0.5 M) in THE (1.250 ml, 0.625 mmol) and MeOH (4 ml). The RM was stirred at RT for 7 h, solid NaBH₃CN (39 mg, 0.621 mmol) was added and the RM was stirred at RT for 20 h. The mixture was concentrated and the residue, containing the title compound, was directly used for the next step without further purification.

Method D: Rt=0.83 min; [M+H]⁺=567.

### Step 5: 4-(3-chloro-4-((1-(piperidin-4-ylmethyl)piperidin-4-yl)oxy)phenyl)-2-methyl-2,7-naphthyridin-1(2H)-one

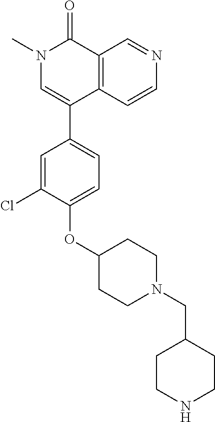 [SMILES: Cn1cc(-c2ccc(OC3CCN(CC4CCNCC4)CC3)c(Cl)c2)c2ccncc2c1=O]

To a 10 ml round bottom flask were added tert-butyl 4-((4-(2-chloro-4-(2-methyl-1-oxo-1,2-dihydro-2,7-naphthyridin-4-yl)phenoxy)piperidin-1-yl)methyl)piperidine-1-carboxylate (0.567 mmol), TFA (1 ml, 12.98 mmol) and DCM (4 ml). The RM was stirred at RT for 2 h, concentrated and the residue was purified by reversed phase chromatography on a REDISEP® Gold HP C18 column (15.5 g) eluting with ACN (from 2% to 100%) in an aq. solution of TFA (0.1%), yielding the corresponding TFA salt of the title compound as a solid (226 mg).

Method D: Rt=0.53 min; [M+H]⁺=467.

### Step 6: 1-(5-(4-((4-(2-chloro-4-(2-methyl-1-oxo-1,2-dihydro-2,7-naphthyridin-4-yl)phenoxy)piperidin-1-yl)methyl)piperidine-1-carbonyl)-2-methoxyphenyl)dihydropyrimidine-2,4(1H,3H)-dione

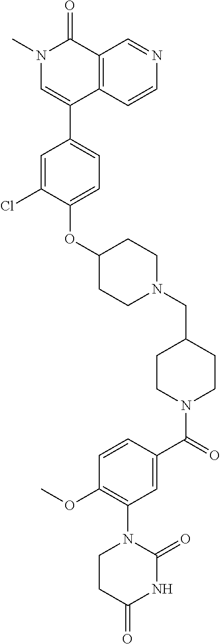 [SMILES: COc1ccc(C(=O)N2CCC(CN3CCC(Oc4ccc(-c5cn(C)c(=O)c6cnccc56)cc4Cl)CC3)CC2)cc1N1CCC(=O)NC1=O]

To a 50 ml round bottom flask were added HATU (63 mg, 0.166 mmol), 3-(2,4-dioxotetrahydropyrimidin-1(2H)-yl)-4-methoxybenzoic acid (intermediate 25, 40 mg, 0.151 mmol), DIPEA (0.100 ml, 0.573 mmol) and DMF (1 ml). The RM was stirred at RT for 30 min, a solution of 4-(3-chloro-4-((1-(piperidin-4-ylmethyl)piperidin-4-yl)oxy)phenyl)-2-methyl-2,7-naphthyridin-1(2H)-one TFA salt (96 mg, 0.138 mmol), DIPEA (0.100 ml, 0.573 mmol) and DMF (0.5 ml) was added and the RM was stirred at RT overnight. The mixture was directly purified by reversed phase chromatography on a REDISEP® Gold HP C18 column (15.5 g) eluting with ACN (from 2% to 100%) in an aq. solution of NH₄HCO₃ (0.1%), yielding the title compound as a solid (51 mg).

Method L: Rt=2.84 min; [M+H]⁺=713.

¹H NMR (400 MHz, DMSO-d₆) δ 10.35 (s, 1H), 9.45 (s, 1H), 8.74 (s, 1H), 7.84 (s, 1H), 7.62-7.13 (m, 7H), 4.51 (m, 3H), 3.68 (m, 10H), 2.71 (m, 4H), 2.37-0.83 (m, 13H).

### Compound A18:1-(2-Chloro-5-(4-((1-(2,5-dimethoxy-4-(1,4,5-trimethyl-6-oxo-1,6-dihydropyridin-3-yl)benzyl)piperidin-4-yl)oxy)piperidine-1-carbonyl)phenyl)dihydropyrimidine-2,4(1H,3H)-dione

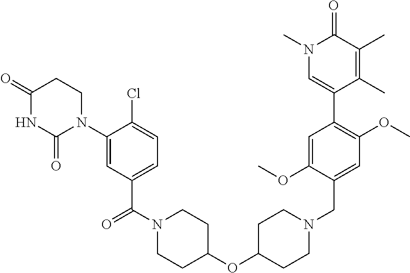 [SMILES: COc1cc(-c2cn(C)c(=O)c(C)c2C)c(OC)cc1CN1CCC(OC2CCN(C(=O)c3ccc(Cl)c(N4CCC(=O)NC4=O)c3)CC2)CC1]

### Step 1: 2,5-dimethoxy-4-(4,4,5,5-tetramethyl-1,3,2-dioxaborolan-2-yl)benzaldehyde

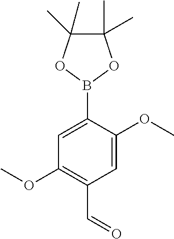

To a 100 ml round bottom flask were added under an argon atmosphere 4-bromo-2,5-dimethoxybenzaldehyde (2.0 g, 8.16 mmol), BISPIN (2.5 g, 7.27 mmol), KOAc (2403 mg, 24.48 mmol) and 1,4-dioxane (30 ml). Solid PdCl₂(dppf)-CH₂Cl₂ (333 mg, 0.408 mmol) was added and the RM was stirred at 90° C. overnight. BISPIN (1000 mg, 3.94 mmol) and PdCl₂(dppf)-CH₂Cl₂ (333 mg, 0.408 mmol) were added and the RM was stirred at 100° C. for 4 h. The RM was cooled to RT, filtered over CELITE®, the solids were washed with EtOAc and the combined filtrates were washed with an aq. solution of HCl (0.1 M) and brine. The organic phase was dried over MgSO₄ and the residue was purified by chromatography on silica gel eluting with EtOAc (from 0% to 40%) in CHX, yielding the title compound as a solid (1.40 g).

Method D: Rt=1.12 min; [M+H]⁺=293.

### Step 2: 2,5-dimethoxy-4-(1,4,5-trimethyl-6-oxo-1,6-dihydropyridin-3-yl)benzaldehyde

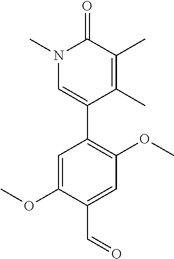

To a 100 ml round bottom flask were added under an argon atmosphere 2,5-dimethoxy-4-(4,4,5,5-tetramethyl-1,3,2-dioxaborolan-2-yl)benzaldehyde (500 mg, 1.626 mmol), 5-bromo-1,3,4-trimethylpyridin-2(1H)-one (intermediate 3, 300 mg, 1.388 mmol), an aq. solution of NaOAc (2 M, 2.083 ml, 4.17 mmol) and DMF (10 ml). Solid PdCl₂(dppf)-CH₂Cl₂ (103 mg, 0.139 mmol) was added and the RM was stirred at 100° C. for 1 h. The mixture was cooled to RT, filtered through CELITE®, the solids were washed with EtOAc, the combined filtrates were concentrated and the residue was purified by reversed phase chromatography on a REDISEP® Gold HP C18 column (15.5 g) eluting with ACN (from 1% to 100%) in an aq. solution of TFA (0.1%), yielding the title compound as a solid (440 mg).

Method D: Rt=0.87 min; [M+H]⁺=302.

### Step 3: 1-(2-chloro-5-(4-((1-(2,5-dimethoxy-4-(1,4,5-trimethyl-6-oxo-1,6-dihydropyridin-3-yl)benzyl)piperidin-4-yl)oxy)piperidine-1-carbonyl)phenyl)dihydropyrimidine-2,4(1H,3H)-dione

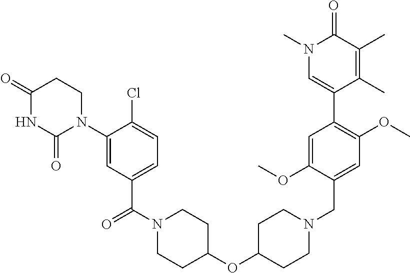 [SMILES: COc1cc(-c2cn(C)c(=O)c(C)c2C)c(OC)cc1CN1CCC(OC2CCN(C(=O)c3ccc(Cl)c(N4CCC(=O)NC4=O)c3)CC2)CC1]

To a 10 ml round bottom flask were added 1-(2-chloro-5-(4-(piperidin-4-yloxy)piperidine-1-carbonyl)phenyl)dihydropyrimidine-2,4(1H,3H)-dione hydrochloride (intermediate 28, 148 mg, 0.292 mmol), 2,5-dimethoxy-4-(1,4,5-trimethyl-6-oxo-1,6-dihydropyridin-3-yl)benzaldehyde (90 mg, 0.266 mmol), TEA (0.100 ml, 0.717 mmol), a solution of ZnCl₂ (0.5 M) in THF (0.6 ml, 0.300 mmol) and MeOH (2.5 ml). The RM was stirred at RT for 7 h, solid NaBH₃CN (20 mg, 0.318 mmol) was added and the RM was stirred at RT overnight. The mixture was directly purified by reversed phase chromatography on a REDISEP® Gold HP C18 column (15.5 g) eluting with ACN (from 0% to 100%) in an aq. solution of NH₄HCO₃ (0.1%), followed by a purification using SFC on a Reprospher PEI column (250×30 mm, 100 Å, 5 m) eluting with MeOH (from 20% to 28%) in CO₂, yielding the title compound as a solid (113 mg).

Method L: Rt=2.95 min; [M+H]⁺=720.

¹H NMR (400 MHz, DMSO-d₆) δ 10.52 (s, 1H), 7.66 (d, J=8.2 Hz, 1H), 7.59 (d, J=2.0 Hz, 1H), 7.45-7.36 (m, 2H), 7.05 (s, 1H), 6.76 (s, 1H), 4.08-3.41 (m, 18H), 2.81-2.69 (m, 4H), 2.16 (m, 2H), 2.04 (s, 3H), 1.85 (m, 8H), 1.49 (m, 4H).

### Compound A19: 1-(2-Chloro-5-(4-((4-((2,6-dimethoxy-4-(1,4,5-trimethyl-6-oxo-1,6-dihydropyridin-3-yl)phenoxy)methyl)piperidin-1-yl)methyl)piperidine-1-carbonyl)phenyl)dihydropyrimidine-2,4(1H,3H)-dione

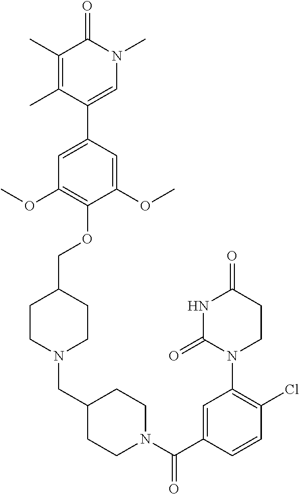 [SMILES: COc1cc(-c2cn(C)c(=O)c(C)c2C)cc(OC)c1OCC1CCN(CC2CCN(C(=O)c3ccc(Cl)c(N4CCC(=O)NC4=O)c3)CC2)CC1]

### Step 1:4-((2,6-dimethoxy-4-(4,4,5,5-tetramethyl-1,3,2-dioxaborolan-2-yl)phenoxy)methyl)piperidine

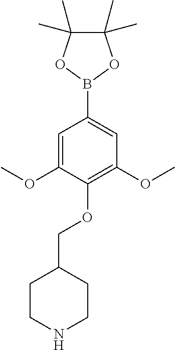

To a 25 ml round bottom flask were added tert-butyl tert-butyl 4-((2,6-dimethoxy-4-(4,4,5,5-tetramethyl-1,3,2-dioxaborolan-2-yl)phenoxy)methyl)piperidine-1-carboxylate (intermediate 18, 232 mg, 0.462 mmol), TFA (0.5 ml, 5.49 mmol) and DCM (4 ml). The RM was stirred at RT for 1 h and concentrated to dryness, yielding the corresponding TFA salt of the title compound as a solid (255 mg).

Method D: Rt=0.85 min; [M+H]⁺=378.

### Step 2: tert-butyl 4-((4-((2,6-dimethoxy-4-(4,4,5,5-tetramethyl-1,3,2-dioxaborolan-2-yl)phenoxy)methyl)piperidin-1-yl)methyl)piperidine-1-carboxylate

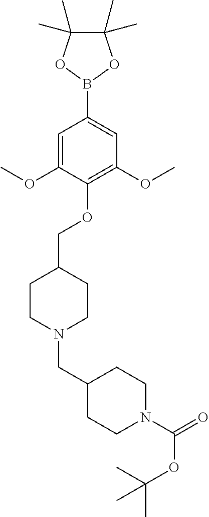 [SMILES: COc1cc(B2OC(C)(C)C(C)(C)O2)cc(OC)c1OCC1CCN(CC2CCN(C(=O)OC(C)(C)C)CC2)CC1]

To a 10 ml round bottom flask were added 4-((2,6-dimethoxy-4-(4,4,5,5-tetramethyl-1,3,2-dioxaborolan-2-yl)phenoxy)methyl)piperidine TFA salt (255 mg, 0.462 mmol), tert-butyl 4-formylpiperidine-1-carboxylate (118 mg, 0.554 mmol), TEA (0.200 ml, 1.435 mmol), a solution of ZnCl₂ (0.5 M) in THE (1 ml, 0.500 mmol) and MeOH (4 ml). The RM was stirred at RT for 7 h, solid NaBH₃CN (30 mg, 0.477 mmol) was added and the RM was stirred at RT for 2 days. The RM was concentrated and the residue, containing the title compound, was directly used for the next step without further purification.

Method D: Rt=1.04 min; [M+H]⁺=575.

### Step 3: 4-((2,6-dimethoxy-4-(4,4,5,5-tetramethyl-1,3,2-dioxaborolan-2-yl)phenoxy)methyl)-1-(piperidin-4-ylmethyl)piperidine

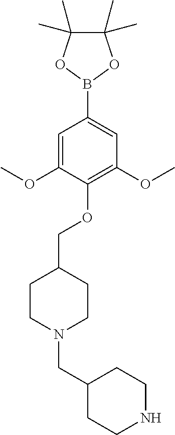

To a 25 ml round bottom flask were added tert-butyl tert-butyl 4-((4-((2,6-dimethoxy-4-(4,4,5,5-tetramethyl-1,3,2-dioxaborolan-2-yl)phenoxy)methyl)piperidin-1-yl)methyl)piperidine-1-carboxylate (0.462 mmol), TFA (1 ml, 12.98 mmol) and DCM (4 ml). The RM was stirred at RT for 1 h, concentrated and the residue was purified by reversed phase chromatography on a REDISEP® Gold HP C18 column (15.5 g) eluting with ACN (from 2% to 100%) in an aq. solution of TFA (0.1%), yielding the corresponding TFA salt of the title compound as a solid (284 mg).

Method D: Rt=0.70 min; [M+H]⁺=475.

### Step 4: (4-((1-((1-(4-chloro-3-(2,4-dioxotetrahydropyrimidin-1(2H)-yl)benzoyl)piperidin-4-yl)methyl)piperidin-4-yl)methoxy)-3,5-dimethoxyphenyl)boronic Acid

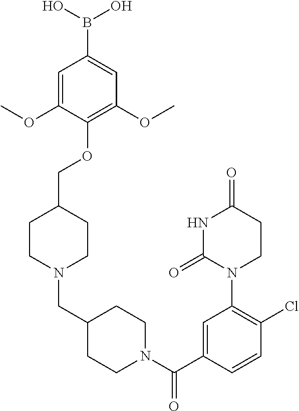 [SMILES: COc1cc(B(O)O)cc(OC)c1OCC1CCN(CC2CCN(C(=O)c3ccc(Cl)c(N4CCC(=O)NC4=O)c3)CC2)CC1]

To a 50 ml round bottom flask were added HATU (101 mg, 0.266 mmol), 4-chloro-3-(2,4-dioxotetrahydropyrimidin-1(2H)-yl)benzoic acid (intermediate 24, 66 mg, 0.246 mmol), DIPEA (0.100 ml, 0.573 mmol) and DMF (1 ml). The RM was stirred at RT for 30 min, a solution of 4-((2,6-dimethoxy-4-(4,4,5,5-tetramethyl-1,3,2-dioxaborolan-2-yl)phenoxy)methyl)-1-(piperidin-4-ylmethyl)piperidine TFA salt (157 mg, 0.221 mmol), DIPEA (0.100 ml, 0.573 mmol) and DMF (1 ml) was added and the RM was stirred at RT for 2 h. The mixture was directly purified by reversed phase chromatography on a REDISEP® Gold HP C18 column (15.5 g) eluting with ACN (from 2% to 100%) in an aq. solution of TFA (0.1%), yielding the corresponding TFA salt of the title compound as a solid (100 mg).

Method D: Rt=0.57 min; [M+H]⁺=643.

### Step 5: 1-(2-chloro-5-(4-((4-((2,6-dimethoxy-4-(1,4,5-trimethyl-6-oxo-1,6-dihydropyridin-3-yl)phenoxy)methyl)piperidin-1-yl)methyl)piperidine-1-carbonyl)phenyl)dihydropyrimidine-2,4(1H,3H)-dione

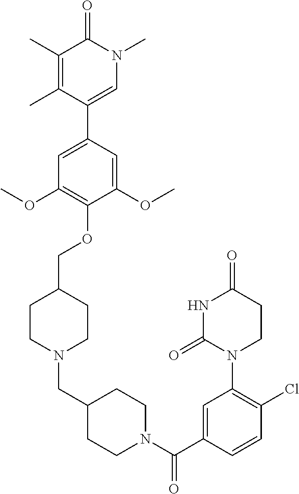 [SMILES: COc1cc(-c2cn(C)c(=O)c(C)c2C)cc(OC)c1OCC1CCN(CC2CCN(C(=O)c3ccc(Cl)c(N4CCC(=O)NC4=O)c3)CC2)CC1]

To a 10 ml round bottom flask were added under an argon atmosphere 5-bromo-1,3,4-trimethylpyridin-2(1H)-one (intermediate 3, 35 mg, 0.159 mmol), (4-((1-((1-(4-chloro-3-(2,4-dioxotetrahydropyrimidin-1(2H)-yl)benzoyl)piperidin-4-yl)methyl)piperidin-4-yl)methoxy)-3,5-dimethoxyphenyl)boronic acid TFA salt (100 mg, 0.137 mmol), Na₂CO₃ (43.5 mg, 0.411 mmol), 1,4-dioxane (2 ml) and water (0.5 ml). Solid PdCl₂(dppf)-CH₂Cl₂ (10 mg, 0.014 mmol) was added and the RM was stirred at 85° C. for 1 h. The mixture was filtered over CELITE®, the solids were washed with EtOAc, the combined filtrates were concentrated and the residue was purified by reversed phase chromatography on a REDISEP® Gold HP C18 column (15.5 g) eluting with ACN (from 1% to 100%) in an aq. solution of NH₄HCO₃ (0.1%), yielding the title compound as a solid (33 mg).

Method L: Rt=2.40 min; [M+H]⁺=734.

¹H NMR (400 MHz, DMSO-d₆) δ 10.51 (m, 1H), 7.65 (d, J=8.2 Hz, 1H), 7.56 (d, J=2.1 Hz, 1H), 7.49 (s, 1H), 7.39 (dd, J=8.3, 2.2 Hz, 1H), 6.55 (s, 2H), 4.45 (m, 1H), 3.78-3.61 (m, 10H), 3.46 (s, 3H), 3.22-2.71 (m, 7H), 2.15 (m, 2H), 2.06 (m, 6H), 1.95-1.56 (m, 8H), 1.34-0.97 (m, 4H).

### Compound A20: 1-(5-(4-((4-(2,5-Dimethoxy-4-(2-methyl-1-oxo-1,2-dihydro-2,7-naphthyridin-4-yl)phenoxy)piperidin-1-yl)methyl)piperidine-1-carbonyl)-2-methoxyphenyl)dihydropyrimidine-2,4(1H,3H)-dione

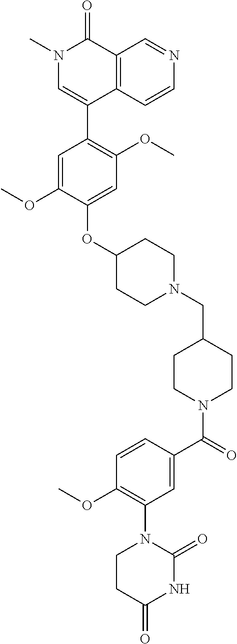 [SMILES: COc1cc(-c2cn(C)c(=O)c3cnccc23)c(OC)cc1OC1CCN(CC2CCN(C(=O)c3ccc(OC)c(N4CCC(=O)NC4=O)c3)CC2)CC1]

### Step 1: (4-bromo-2,5-dimethoxyphenoxy)(tert-butyl)dimethylsilane

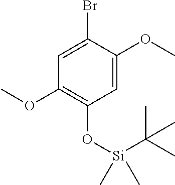 [SMILES: COc1cc(O[Si](C)(C)C(C)(C)C)c(OC)cc1Br]

To a 100 ml round bottom flask were added 4-bromo-2,5-dimethoxybenzaldehyde (3.75 g, 15.30 mmol), meta-chloroperbenzoic acid (3.96 g, 22.95 mmol) and DCM (50 ml). The RM was stirred at RT for 3 h. The mixture was poured into a mixture of EtOAc and an aq. sat. solution of NaHCO₃, the aq. phase was extracted with EtOAc, the combined organic phases were washed with brine, dried over MgSO₄ and concentrated. The residue was diluted with MeOH (50 ml), an aq. solution of NaOH (1 M, 15.30 ml, 15.30 mmol) was added and the RM was stirred at RT for 3 h. The mixture was diluted with EtOAc, the organic phase was washed with an aq. sat. solution of NH₄Cl and brine, dried over MgSO₄ and concentrated. The residue was diluted with DCM (50 ml), imidazole (1.56 g, 22.95 mmol) was added and the mixture was stirred at RT for 15 minutes. Tert-butyldimethylsilyl chloride (2.54 g, 16.83 mmol) was added and the suspension was stirred at RT for 1 h. The mixture was diluted with EtOAc, the organic phase was washed with an aq. sat. solution of NH₄Cl and brine, dried over MgSO₄ and concentrated. The residue was purified by chromatography on silica gel eluting with EtOAc (from 0% to 5%) in CHX, yielding the title compound as a solid (4.49 g).

Method M: Rt=1.46 min; [M+H]⁺=347, 349.

### Step 2: tert-butyl(2,5-dimethoxy-4-(4,4,5,5-tetramethyl-1,3,2-dioxaborolan-2-yl)phenoxy)dimethylsilane

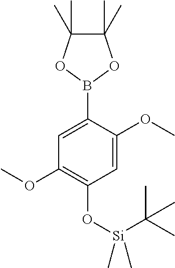 [SMILES: COc1cc(B2OC(C)(C)C(C)(C)O2)c(OC)cc1O[Si](C)(C)C(C)(C)C]

To a 100 ml round bottom flask were added (4-bromo-2,5-dimethoxyphenoxy)(tert-butyl)dimethylsilane (1.59 g, 4.34 mmol), palladium(II) acetate (49 mg, 0.218 mmol), (oxybis(2,1-phenylene))bis(diphenylphosphane) (233 mg, 0.434 mmol), TEA (2.42 ml, 17.36 mmol), BISPIN (1.9 ml, 13.09 mmol) and 1,4-dioxane (30 ml) and the RM was stirred at 80° C. overnight. The mixture was diluted with an aq. sat. solution of NH₄Cl and extracted with EtOAc. The combined organic phases were washed with water and brine, dried over MgSO₄ and the residue was purified by chromatography on silica gel eluting with EtOAc (from 0% to 30%) in CHX, yielding the title compound as a solid (403 mg).

Method M: Rt=1.43 min; [M+H]⁺=395.

### Step 3: 4-(4-((tert-butyldimethylsilyl)oxy)-2,5-dimethoxyphenyl)-2-methyl-2,7-naphthyridin-1(2H)-one

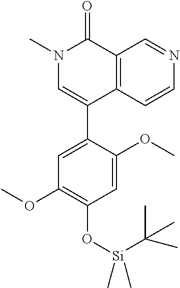 [SMILES: COc1cc(-c2cn(C)c(=O)c3cnccc23)c(OC)cc1O[Si](C)(C)C(C)(C)C]

To a 25 ml round bottom flask were added under an argon atmosphere 4-bromo-2-methyl-2,7-naphthyridin-1(2H)-one (intermediate 5, 700 mg, 0.908 mmol), tert-butyl(2,5-dimethoxy-4-(4,4,5,5-tetramethyl-1,3,2-dioxaborolan-2-yl)phenoxy)dimethylsilane (403 mg, 1.022 mmol), Na₂CO₃ (289 mg, 2.72 mmol), 1,4-dioxane (6 ml) and water (1.5 ml). Solid PdCl₂(dppf) (34 mg, 0.046 mmol) was added and the RM was stirred at 100° C. for 16 h. The mixture was cooled to RT, filtered through CELITE®, the solids were washed with EtOAc, the combined filtrates were washed with water and brine, dried over MgSO₄ and the residue was purified by chromatography on silica gel eluting with MeOH (from 0% to 10%) in DCM, yielding the title compound as a solid (365 mg).

Method M: Rt=1.23 min; [M+H]⁺=427.

### Step 4: 4-(4-hydroxy-2,5-dimethoxyphenyl)-2-methyl-2,7-naphthyridin-1(2H)-one

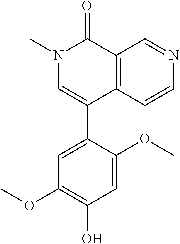

To a 25 ml round bottom flask were added 4-(4-((tert-butyldimethylsilyl)oxy)-2,5-dimethoxyphenyl)-2-methyl-2,7-naphthyridin-1(2H)-one (365 mg, 0.565 mmol), TEA (0.236 ml, 1.694 mmol) and THE (4 ml). A solution of TBAF (1 M) in THF (1.129 ml, 1.129 mmol) was added and the RM was stirred at RT for 30 min. The mixture was concentrated and the residue was purified by chromatography on silica gel eluting with MeOH (from 0% to 10%) in DCM, yielding the title compound as a solid (258 mg).

Method M: Rt=0.65 min; [M+H]⁺=313.

### Step 5:4-(2,5-dimethoxy-4-(piperidin-4-yloxy)phenyl)-2-methyl-2,7-naphthyridin-1(2H)-one

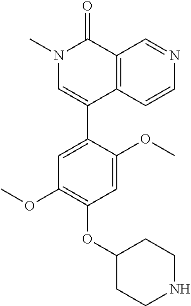 [SMILES: COc1cc(-c2cn(C)c(=O)c3cnccc23)c(OC)cc1OC1CCNCC1]

To a 50 ml round bottom flask were added under an argon atmosphere 4-(4-hydroxy-2,5-dimethoxyphenyl)-2-methyl-2,7-naphthyridin-1(2H)-one (62 mg, 0.199 mmol), tert-butyl 4-hydroxypiperidine-1-carboxylate (48 mg, 0.234 mmol), PPh₃ (62 mg, 0.236 mmol) and THE (2 ml). DIAD (0.046 ml, 0.238 mmol) was added dropwise and the RM was stirred at 60° C. for 3 hours. The mixture was concentrated, the residue was diluted in DCM (2 ml), TFA (0.400 ml, 5.19 mmol) was added and the RM was stirred at RT for 1 h, concentrated and the residue was purified by reversed phase chromatography on a REDISEP® Gold HP C18 column (15.5 g) eluting with ACN (from 2% to 100%) in an aq. solution of TFA (0.1%), yielding the corresponding TFA salt of the title compound as a solid (53 mg).

Method M: Rt=0.48 min; [M+H]⁺=396.

### Step 6: 4-(2,5-dimethoxy-4-((1-(piperidin-4-ylmethyl)piperidin-4-yl)oxy)phenyl)-2-methyl-2,7-naphthyridin-1(2H)-one

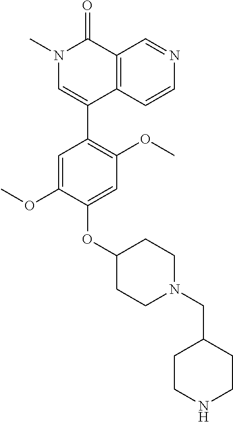 [SMILES: COc1cc(-c2cn(C)c(=O)c3cnccc23)c(OC)cc1OC1CCN(CC2CCNCC2)CC1]

To a 10 ml round bottom flask were added 4-(2,5-dimethoxy-4-(piperidin-4-yloxy)phenyl)-2-methyl-2,7-naphthyridin-1(2H)-one TFA salt (51 mg, 0.094 mmol), tert-butyl 4-formylpiperidine-1-carboxylate (23 mg, 0.108 mmol), TEA (0.050 ml, 0.359 mmol), a solution of ZnCl₂ (0.5 M) in THE (0.200 ml, 0.100 mmol) and MeOH (1.5 ml). The RM was stirred at RT for 7 h, solid NaBH₃CN (6 mg, 0.095 mmol) was added and the RM was stirred at RT for 20 h. The mixture was concentrated, TFA (0.200 ml, 2.60 mmol) and DCM (1.5 ml) were added and the RM was stirred at RT for 1 h, concentrated and the residue was purified by reversed phase chromatography on a REDISEP® Gold HP C18 column (15.5 g) eluting with ACN (from 1% to 100%) in an aq. solution of TFA (0.1%), yielding the corresponding TFA salt of the title compound as a solid (50 mg).

Method M: Rt=0.26 min; [M+H]⁺=493.

### Step 7:1-(5-(4-((4-(2,5-dimethoxy-4-(2-methyl-1-oxo-1,2-dihydro-2,7-naphthyridin-4-yl)phenoxy)piperidin-1-yl)methyl)piperidine-1-carbonyl)-2-methoxyphenyl)dihydropyrimidine-2,4(1H,3H)-dione

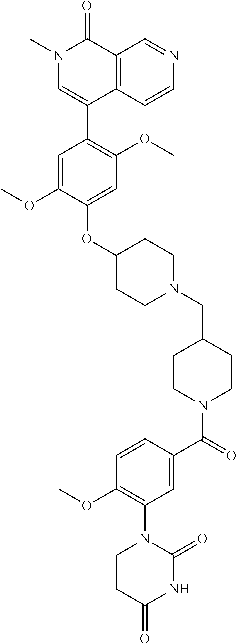 [SMILES: COc1cc(-c2cn(C)c(=O)c3cnccc23)c(OC)cc1OC1CCN(CC2CCN(C(=O)c3ccc(OC)c(N4CCC(=O)NC4=O)c3)CC2)CC1]

To a 50 m round bottom as were added HATU (30 mg, 0.079 mmol), 3-(2,4-dioxotetrahydropyrimidin-1(2H)-yl)-4-methoxybenzoic acid (intermediate 25, 20 mg, 0.076 mmol), DIPEA (0.050 ml, 0.286 mmol) and DMF (0.5 ml). The RM was stirred at RT for 30 min, a solution of 4-(2,5-dimethoxy-4-((1-(piperidin-4-ylmethyl)piperidin-4-yl)oxy)phenyl)-2-methyl-2,7-naphthyridin-1(2H)-one TFA salt (50 mg, 0.069 mmol), DIPEA (0.050 ml, 0.286 mmol) and DMF (1 ml) was added and the RM was stirred at RT for 30 minutes. The mixture was directly purified by reversed phase chromatography on a REDISEP® Gold HP C18 column (15.5 g) eluting with ACN (from 1% to 100%) in an aq. solution of NH₄HCO₃ (0.1%), followed by a purification using SFC on a Waters Viridis 2-EP column (250×30 mm, 130 Å, 5 m) eluting with MeOH (from 19% to 27%) in CO₂, yielding the title compound as a solid (26.7 mg).

Method N: Rt=0.69 min; [M+H]⁺=739.

¹H NMR (400 MHz, DMSO-d₆) δ 10.35 (s, 1H), 9.45-9.38 (m, 1H), 8.65 (d, J=5.6 Hz, 1H), 7.70 (s, 1H), 7.39 (d, J=8.3 Hz, 1H), 7.34 (d, J=2.1 Hz, 1H), 7.22-7.11 (m, 2H), 6.99 (m, 2H), 4.46 (m, 1H), 3.86 (s, 3H), 3.75 (s, 3H), 3.65 (s, 3H), 3.61 (t, J=6.6 Hz, 2H), 3.58 (s, 3H), 3.32-3.29 (m, 6H), 3.11 (m, 2H), 2.70 (t, J=6.7 Hz, 2H), 2.42-0.94 (m, 11H).

### Compound A21: 1-(2-Chloro-5-(4-((1-(2,6-dimethoxy-4-(1,4,5-trimethyl-6-oxo-1,6-dihydropyridin-3-yl)phenethyl)piperidin-4-yl)oxy)piperidine-1-carbonyl)phenyl)dihydropyrimidine-2,4(1H,3H)-dione

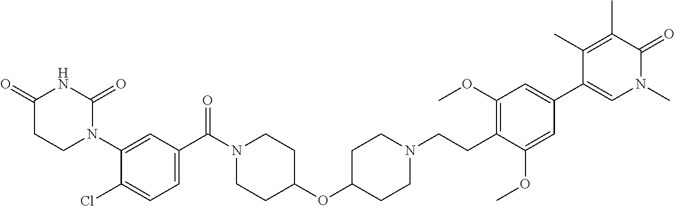 [SMILES: COc1cc(-c2cn(C)c(=O)c(C)c2C)cc(OC)c1CCN1CCC(OC2CCN(C(=O)c3ccc(Cl)c(N4CCC(=O)NC4=O)c3)CC2)CC1]

### Step 1: 5-bromo-1,3-dimethoxy-2-(2-methoxyvinyl)benzene

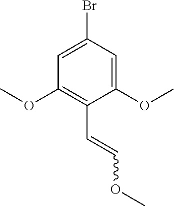

To a 100 ml round bottom flask were added (methoxymethyl)triphenylphosphonium chloride (21 g, 20.5 mmol), t-BuOK (9.2 g, 82 mmol) and TH (100 ml). The RM was stirred at 0° C. for 30 min and 4-bromo-2,6-dimethoxybenzaldehyde (5 g, 20.5 mmol) was added. The RM was stirred at 0° C. for 1 h, then at 70° C. for 16 h. The mixture was added into water (100 ml), extracted with EtOAc (2×100 ml), the combined organic phases were washed with brine (2×50 mL), dried over Na₂SO₄ and the residue was purified by chromatography on silica gel eluting with EtOAc (from 20% to 50%) in PE, yielding the title compound as a solid (2.3 g).

Method H: Rt=2.11 min; [M+H]⁺=273, 275.

### Step 2: 2-(4-bromo-2,6-dimethoxyphenyl)acetaldehyde

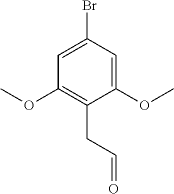

To a 250 ml round bottom flask were added 5-bromo-1,3-dimethoxy-2-(2-methoxyvinyl)benzene (2.3 g, 8.46 mmol), acetone (40 ml) and an aq. solution of HCl (2 M, 4 ml). The RM was stirred at 65° C. for 3 h and concentrated to give the title compound as an oil (2.3 g), which was directly used for the next step without further purification.

Method H: Rt=2.08 min; [M+H]⁺=259, 261.

### Step 3: tert-butyl 4-((1-(4-bromo-2,6-dimethoxyphenethyl)piperidin-4-yl)oxy)piperidine-1-carboxylate

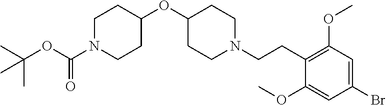 [SMILES: COc1cc(Br)cc(OC)c1CCN1CCC(OC2CCN(C(=O)OC(C)(C)C)CC2)CC1]

To a 250 ml round bottom flask were added 2-(4-bromo-2,6-dimethoxyphenyl)acetaldehyde (2.3 g, 8.88 mmol), tert-butyl 4-(piperidin-4-yloxy)piperidine-1-carboxylate (intermediate 1, 3.26 g, 10.65 mmol), a solution of ZnCl₂ (1 M) in THE (12 ml, 12 mmol) and DMSO (30 ml). The RM was stirred at RT for 1 h and solid NaBH₃CN (1.12 g, 17.76 mmol) was added. The RM was stirred at RT for 16 h, the solvent was removed and the residue was purified by reversed phase chromatography on a Biotage Agela C18 column (120 g, spherical 20-35 μm, 100 Å) eluting with ACN (from 5% to 95%) in an aq. solution of TFA (0.1%), yielding the title compound as a solid (2.1 g).

Method A: Rt=1.39 min; [M+H]⁺=527, 529.

### Step 4: tert-butyl 4-((1-(2,6-dimethoxy-4-(4,4,5,5-tetramethyl-1,3,2-dioxaborolan-2-yl)phenethyl)piperidin-4-yl)oxy)piperidine-1-carboxylate

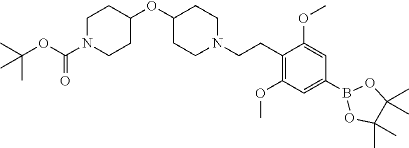 [SMILES: COc1cc(B2OC(C)(C)C(C)(C)O2)cc(OC)c1CCN1CCC(OC2CCN(C(=O)OC(C)(C)C)CC2)CC1]

To a 100 ml round bottom flask were added under an argon atmosphere tert-butyl 4-((1-(4-bromo-2,6-dimethoxyphenethyl)piperidin-4-yl)oxy)piperidine-1-carboxylate (2.1 g, 4 mmol), BISPIN (1.32 g, 5.2 mmol), K₂CO₃ (1.38 g, 10 mmol), 1,4-dioxane (20 ml) and PdCl₂(dppf) (146 mg, 0.2 mmol) and the RM was stirred at 100° C. for 16 h. Water (50 ml) was added, the mixture was extracted with EtOAc (2×75 ml), the combined organic phases were washed with brine (2×30 mL) and dried over Na₂SO₄. The residue was purified by reversed phase chromatography on a Biotage Agela C18 column (120 g, spherical 20-35 μm, 100 Å) eluting with ACN (from 5% to 95%) in an aq. solution of TFA (0.1%), yielding the title compound as a solid (1.1 g).

Method A: Rt=1.16 min; [M+H]⁺=575.

### Step 5: tert-butyl 4-((1-(2,6-dimethoxy-4-(1,4,5-trimethyl-6-oxo-1,6-dihydropyridin-3-yl)phenethyl)piperidin-4-yl)oxy)piperidine-1-carboxylate

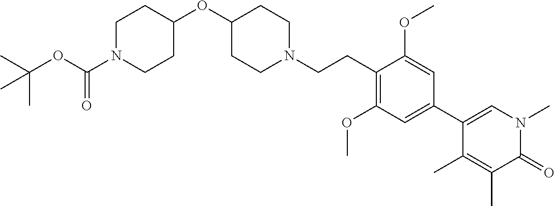 [SMILES: COc1cc(-c2cn(C)c(=O)c(C)c2C)cc(OC)c1CCN1CCC(OC2CCN(C(=O)OC(C)(C)C)CC2)CC1]

To a 250 ml round bottom flask were added under an argon atmosphere tert-butyl 4-((1-(2,6-dimethoxy-4-(4,4,5,5-tetramethyl-1,3,2-dioxaborolan-2-yl)phenethyl)piperidin-4-yl)oxy)piperidine-1-carboxylate (1.1 g, 1.9 mmol), 5-bromo-1,3,4-trimethylpyridin-2(1H)-one (intermediate 3, 414 mg, 1.9 mmol), K₂CO₃ (661 mg, 4.8 mmol), 1,4-dioxane (20 ml), water (4 ml) and PdCl₂(dppf) (70 mg, 0.1 mmol) and the RM was stirred at 90° C. for 16 h. Water (50 ml) was added, the mixture was extracted with EtOAc (2×75 ml), the combined organic phases were washed with brine (2×30 mL), dried over Na₂SO₄ and the residue was purified by reversed phase chromatography on a Biotage Agela C18 column (120 g, spherical 20-35 μm, 100 Å) eluting with ACN (from 5% to 95%) in an aq. solution of TFA (0.1%), yielding the title compound as a solid (1.1 g).

Method A: Rt=1.33 min; [M+H]⁺=584.

### Step 6: 5-(3,5-dimethoxy-4-(2-(4-(piperidin-4-yloxy)piperidin-1-yl)ethyl)phenyl)-1,3,4-trimethylpyridin-2(1H)-one

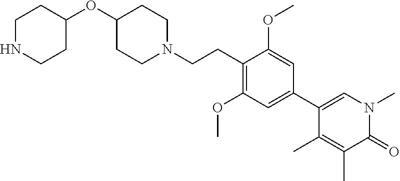 [SMILES: COc1cc(-c2cn(C)c(=O)c(C)c2C)cc(OC)c1CCN1CCC(OC2CCNCC2)CC1]

To a 250 mL round bottom flask were added tert-butyl 4-((1-(2,6-dimethoxy-4-(1,4,5-trimethyl-6-oxo-1,6-dihydropyridin-3-yl)phenethyl)piperidin-4-yl)oxy)piperidine-1-carboxylate (1.1 g, 1.89 mmol), methanol (15 ml) and a solution of HCl (4 M) in 1,4-dioxane (6 ml). The RM was stirred at RT for 3 h and the solvents were removed, yielding the hydrochloride salt of the title compound as an oil, which was used without further purification for the next step.

Method A: Rt=1.06 min; [M+H]⁺=483.

### Step 7:1-(2-chloro-5-(4-((1-(2,6-dimethoxy-4-(1,4,5-trimethyl-6-oxo-1,6-dihydropyridin-3-yl)phenethyl)piperidin-4-yl)oxy)piperidine-1-carbonyl)phenyl)dihydropyrimidine-2,4(1H,3H)-dione

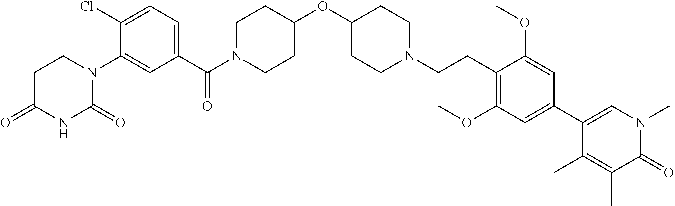 [SMILES: COc1cc(-c2cn(C)c(=O)c(C)c2C)cc(OC)c1CCN1CCC(OC2CCN(C(=O)c3ccc(Cl)c(N4CCC(=O)NC4=O)c3)CC2)CC1]

To a 250 ml round bottom flask were added 5-(3,5-dimethoxy-4-(2-(4-(piperidin-4-yloxy)piperidin-1-yl)ethyl)phenyl)-1,3,4-trimethylpyridin-2(1H)-one hydrochloride (600 mg, 1.24 mmol), 4-chloro-3-(2,4-dioxotetrahydropyrimidin-1(2H)-yl)benzoic acid (intermediate 24, 333 mg 1.24 mmol), DIPEA (640 mg, 4.96 mmol) and DMF (10 ml) and the RM was stirred at rt for 15 min. Solid HATU (708 mg, 1.86 mmol) was added and the RM was stirred at RT for 16 h. The solvent was removed and the residue was purified by preparative HPLC on an XBridge C18 column (21.2×250 mm, 10 μm) eluting with ACN (from 5% to 95%) in an aq. solution of NH₄HCO₃ (10 mM), yielding the title compound as a solid (150 mg).

Method H: Rt=1.76 min; [M+H]⁺=736.

¹H NMR (500 MHz, DMSO-d₆) δ 10.52 (s, 1H), 7.64 (d, J=8.2 Hz, 1H), 7.57 (s, 1H), 7.49 (s, 1H), 7.40 (dd, J=8.2, 2.0 Hz, 1H), 6.50 (s, 2H), 3.98 (s, 1H), 3.77 (s, 6H), 3.64 (dd, J=26.5, 20.3 Hz, 3H), 3.44 (s, 5H), 3.31 (s, 1H), 3.17 (s, 1H), 2.74 (dd, J=13.8, 7.0 Hz, 6H), 2.34 (d, J=27.6 Hz, 2H), 2.04 (s, 8H), 1.80 (s, 4H), 1.44 (s, 4H).

### Compound A22: 1-(3-(4-((1-(2,6-Dimethoxy-4-(1,4,5-trimethyl-6-oxo-1,6-dihydropyridin-3-yl)phenethyl)piperidin-4-yl)oxy)piperidine-1-carbonyl)phenyl)dihydropyrimidine-2,4(1H,3H)-dione

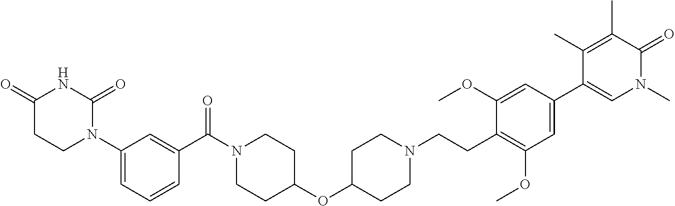 [SMILES: COc1cc(-c2cn(C)c(=O)c(C)c2C)cc(OC)c1CCN1CCC(OC2CCN(C(=O)c3cccc(N4CCC(=O)NC4=O)c3)CC2)CC1]

### Step 1: 2,6-dimethoxy-4-(1,4,5-trimethyl-6-oxo-1,6-dihydropyridin-3-yl)benzaldehyde

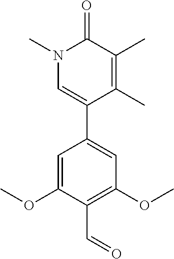

To a 50 ml round bottom flask were added under an argon atmosphere 5-bromo-1,3,4-trimethylpyridin-2(1H)-one (intermediate 3, 400 mg, 1.85 mmol), 2,6-dimethoxy-4-(4,4,5,5-tetramethyl-1,3,2-dioxaborolan-2-yl)benzaldehyde (intermediate 7, 865 mg, 2.96 mmol), Na₂CO₃ (390 mg, 3.67 mmol), PdCl₂(dppf) (134 mg, 0.18 mmol), 1,4-dioxane (10 ml) and water (2 ml) and the RM was stirred at 100° C. for 16 h. The mixture was added into water (20 ml), extracted with EtOAc (3×20 ml), the combined organic phases were washed with brine (20 ml), dried over Na₂SO₄ and the residue was purified by chromatography on silica gel eluting with MeOH (from 0 to 10%) in EtOAc, yielding the title compound as a solid (400 mg).

Method A: Rt=1.40 min; [M+H]⁺=302.

### Step 2: 5-(3,5-dimethoxy-4-(2-methoxyvinyl)phenyl)-1,3,4-trimethylpyridin-2(1H)-one

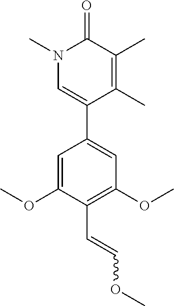

To a 50 ml round bottom flask were added (methoxymethyl)triphenylphosphonium chloride (683 mg, 1.99 mmol), t-BuOK (298 mg, 2.66 mmol) and TH (10 ml). The RM was stirred at 0° C. for 30 min, solid 2,6-dimethoxy-4-(1,4,5-trimethyl-6-oxo-1,6-dihydropyridin-3-yl)benzaldehyde (200 mg, 0.66 mmol) was added and the RM was stirred at 0° C. for 1 h and at 70° C. for 16 h. The mixture was added into water (20 ml), extracted with EtOAc (3×20 ml), the combined organic phases were washed with brine (2×10 ml), dried over Na₂SO₄ and the residue was purified by reversed phase chromatography on a Biotage Agela C18 column (120 g, spherical 20-35 μm, 100 Å) eluting with ACN (from 5% to 80%) in an aq. solution of TFA (0.1%), yielding the title compound as a solid (150 mg).

Method H: Rt=1.79 min; [M+H]⁺=330.

### Step 3: 2-(2,6-dimethoxy-4-(1,4,5-trimethyl-6-oxo-1,6-dihydropyridin-3-yl)phenyl)acetaldehyde

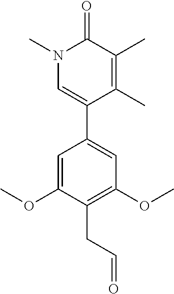

To a 100 ml round bottom flask were added 5-(3,5-dimethoxy-4-(2-methoxyvinyl)phenyl)-1,3,4-trimethylpyridin-2(1H)-one (150 mg, 0.45 mmol), an aq. solution of H₂SO₄ (2 M, 2 ml) and acetone (4 ml). The RM was stirred at 65° C. for 2 h and added into water (20 ml). The mixture was extracted with EtOAc (2×20 ml), the combined organic phases were washed with brine (2×10 ml), dried over Na₂SO₄ and the residue was purified by reversed phase chromatography on a Biotage Agela C18 column (120 g, spherical 20-35 μm, 100 Å) eluting with ACN (from 0% to 80%) in an aq. solution of NH₄HCO₃ (0.1%), yielding the title compound as a solid (80 mg).

Method A: Rt=1.73 min; [M+H]⁺=316.

### Step 4: 1-(3-(4-((1-(2,6-dimethoxy-4-(1,4,5-trimethyl-6-oxo-1,6-dihydropyridin-3-yl)phenethyl)piperidin-4-yl)oxy)piperidine-1-carbonyl)phenyl)dihydropyrimidine-2,4(1H,3H)-dione

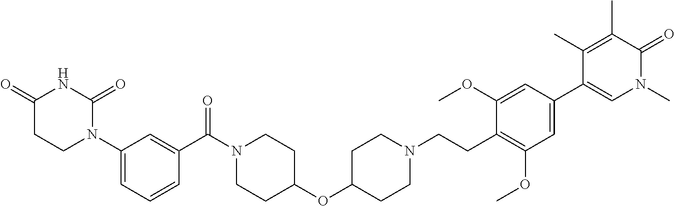 [SMILES: COc1cc(-c2cn(C)c(=O)c(C)c2C)cc(OC)c1CCN1CCC(OC2CCN(C(=O)c3cccc(N4CCC(=O)NC4=O)c3)CC2)CC1]

To a 50 ml round bottom flask were added 2-(2,6-dimethoxy-4-(1,4,5-trimethyl-6-oxo-1,6-dihydropyridin-3-yl)phenyl)acetaldehyde (80 mg, 0.25 mmol), 1-(3-(4-(piperidin-4-yloxy)piperidine-1-carbonyl)phenyl)dihydropyrimidine-2,4(1H,3H)-dione hydrochloride (intermediate 27,122 mg, 0.30 mmol), K₂CO₃ (35 mg, 0.25 mmol), DMSO (6 ml) and a solution of ZnCl₂ (1.0 M) in THE (0.33 ml, 0.33 mmol). The RM was stirred at RT for 1 h, solid NaBH₃CN (24 mg, 0.38 mmol) was added and the RM was stirred at RT for 16 h. The mixture was concentrated and the residue was purified by preparative HPLC on an Xtimate C18 column (250×21.2 mm, 10 μm) eluting with ACN (from 5% to 80%) in an aq. solution of NH₄HCO₃ (10 mM), yielding the title compound as a solid (45 mg).

Method H: Rt=1.67 min; [M+H]⁺=700.

¹H NMR (500 MHz, DMSO-d₆) δ 7.54-7.18 (m, 4H), 6.49 (s, 2H), 3.95 (d, J=42.2 Hz, 1H), 3.94-3.76 (m, 5H), 3.73-3.61 (m, 1H), 3.58-3.39 (m, 4H), 3.33 (s, 3H), 3.20 (d, J=34.7 Hz, 2H), 2.83-2.64 (m, 5H), 2.54-2.43 (m, 6H), 2.34-2.25 (m, 2H), 2.12-1.93 (m, 7H), 2.00-1.67 (m, 4H), 1.42 (d, J=9.1 Hz, 3H).

### Compound A23: 1-(5-(4-((4-(2,5-Dimethoxy-4-(2-methyl-1-oxo-1,2-dihydro-2,7-naphthyridin-4-yl)benzyl)piperazin-1-yl)methyl)piperidine-1-carbonyl)-2-methoxyphenyl)dihydropyrimidine-2,4(1H,3H)-dione

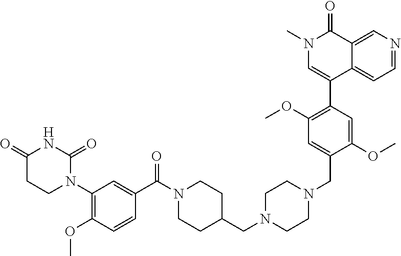 [SMILES: COc1cc(-c2cn(C)c(=O)c3cnccc23)c(OC)cc1CN1CCN(CC2CCN(C(=O)c3ccc(OC)c(N4CCC(=O)NC4=O)c3)CC2)CC1]

### Step 1: tert-buty 4-((4-(2,5-dimethoxy-4-(2-methyl-1-oxo-1,2-dihydro-2,7-naphthyridin-4-yl)benzyl)piperazin-1-yl)methyl)piperidine-1-carboxylate

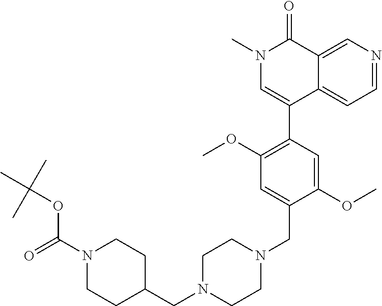 [SMILES: COc1cc(-c2cn(C)c(=O)c3cnccc23)c(OC)cc1CN1CCN(CC2CCN(C(=O)OC(C)(C)C)CC2)CC1]

To a 25 ml round bottom flask was added tert-butyl 4-(piperazin-1-ylmethyl)piperidine-1-carboxylate (intermediate 2, 200 mg, 0.7 mmol), 2,5-dimethoxy-4-(2-methyl-1-oxo-1,2-dihydro-2,7-naphthyridin-4-yl)benzaldehyde (intermediate 12, 227 mg, 0.7 mmol), a solution of ZnCl₂ (1 M) in THE (1 ml, 1 mmol) and DMSO (5 ml). The RM was stirred at RT for 1 h, solid NaBH₃CN (176 mg, 2.8 mmol) and MeOH (2 ml) were added and the RM was stirred at RT for 16 h. The mixture was added into water (50 ml), the mixture was filtered and the solids were washed with water (10 ml) and dried, yielding the title compound as a solid (330 mg).

Method H: Rt=2.10 min; [M+H]⁺=592.

### Step 2: 4-(2,5-dimethoxy-4-((4-(piperidin-4-ylmethyl)piperazin-1-yl)methyl)phenyl)-2-methyl-2,7-naphthyridin-1(2H)-one

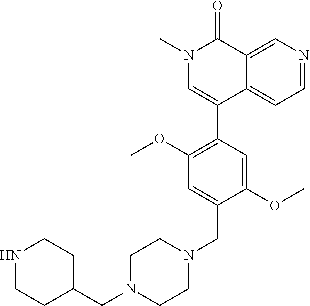 [SMILES: COc1cc(-c2cn(C)c(=O)c3cnccc23)c(OC)cc1CN1CCN(CC2CCNCC2)CC1]

To a 25 ml round bottom as were added tert-butyl 4-((4-(2,5-dimethoxy-4-(2-methyl-1-oxo-1,2-dihydro-2,7-naphthyridin-4-yl)benzyl)piperazin-1-yl)methyl)piperidine-1-carboxylate (330 mg, 0.56 mmol), DCM (20 ml) and a solution of HCl (4 M) in 1,4-dioxane (2 ml). The RM was stirred at RT for 1 h and evaporated to dryness, yielding the corresponding hydrochloride salt of the title compound as a solid (350 mg), which was used for the next step without further purification.

Method B: Rt=1.75 min; [M+H]⁺=492.

### Step 3: 1-(5-(4-((4-(2,5-dimethoxy-4-(2-methyl-1-oxo-1,2-dihydro-2,7-naphthyridin-4-yl)benzyl)piperazin-1-yl)methyl)piperidine-1-carbonyl)-2-methoxyphenyl)dihydropyrimidine-2,4(1H,3H)-dione

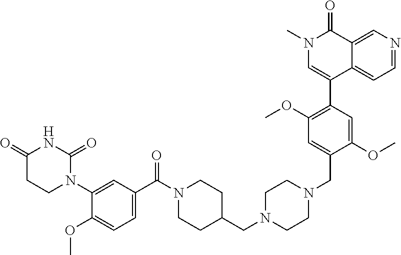 [SMILES: COc1cc(-c2cn(C)c(=O)c3cnccc23)c(OC)cc1CN1CCN(CC2CCN(C(=O)c3ccc(OC)c(N4CCC(=O)NC4=O)c3)CC2)CC1]

To a 25 ml round bottom flask were added 4-(2,5-dimethoxy-4-((4-(piperidin-4-ylmethyl)piperazin-1-yl)methyl)phenyl)-2-methyl-2,7-naphthyridin-1(2H)-one hydrochloride (350 mg, 0.55 mmol), perfluorophenyl 3-(2,4-dioxotetrahydropyrimidin-1(2H)-yl)-4-methoxybenzoate (intermediate 26, 236 mg, 0.55 mmol), DIEA (284 mg, 2.2 mmol) and DMF (4 ml). The RM was stirred at RT for 1 h, the solvent was removed and the residue was purified by preparative HPLC on an XBridge C18 column (250×21.2 mm, 10 μm) eluting with ACN (from 5% to 80%) in an aq. solution of NH₄HCO₃ (10 mM), yielding the title compound as a solid (87 mg).

Method C: Rt=1.56 min; [M+H]⁺=738.

¹H NMR (500 MHz, DMSO-d₆) δ 10.34 (s, 1H), 9.40 (s, 1H), 8.64 (d, J=5.6 Hz, 1H), 7.74 (s, 1H), 7.36 (dd, J=8.4, 2.0 Hz, 1H), 7.32 (d, J=2.1 Hz, 1H), 7.15 (d, J=8.6 Hz, 1H), 7.12 (s, 1H), 7.03 (d, J=5.7 Hz, 1H), 6.92 (s, 1H), 3.84 (s, 3H), 3.74 (s, 3H), 3.67-3.56 (m, 8H), 3.53 (d, J=4.3 Hz, 2H), 2.68 (t, J=6.5 Hz, 2H), 2.50-2.34 (m, 12H), 2.16 (d, J=7.1 Hz, 2H), 1.80-1.66 (m, 3H), 1.14-1.00 (m, 2H).

### Compound A24: 1-(5-(4-((4-(2,6-Dimethoxy-4-(2-methyl-1-oxo-1,2-dihydro-2,7-naphthyridin-4-yl)benzyl)piperazin-1-yl)methyl)piperidine-1-carbonyl)-2-methoxyphenyl)dihydropyrimidine-2,4(1H,3H)-dione

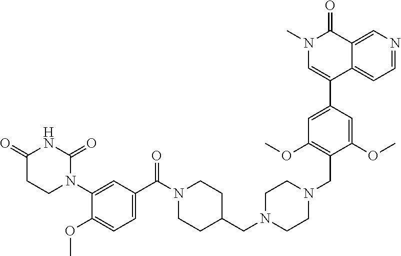 [SMILES: COc1ccc(C(=O)N2CCC(CN3CCN(Cc4c(OC)cc(-c5cn(C)c(=O)c6cnccc56)cc4OC)CC3)CC2)cc1N1CCC(=O)NC1=O]

### Step 1: tert-butyl 4-((4-(2,6-dimethoxy-4-(2-methyl-1-oxo-1,2-dihydro-2,7-naphthyridin-4-yl)benzyl)piperazin-1-yl)methyl)piperidine-1-carboxylate

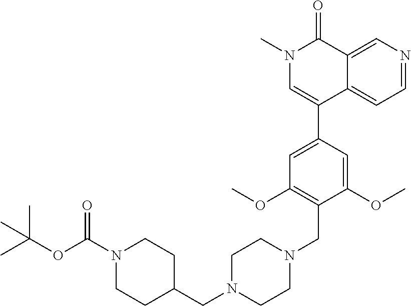 [SMILES: COc1cc(-c2cn(C)c(=O)c3cnccc23)cc(OC)c1CN1CCN(CC2CCN(C(=O)OC(C)(C)C)CC2)CC1]

To a 25 ml round bottom flask were added tert-butyl 4-(piperazin-1-ylmethyl)piperidine-1-carboxylate (intermediate 2, 200 mg, 0.7 mmol), 2,6-dimethoxy-4-(2-methyl-1-oxo-1,2-dihydro-2,7-naphthyridin-4-yl)benzaldehyde (intermediate 11, 227 mg, 0.7 mmol), a solution of ZnCl₂ (1 M) in THE (1 ml, 1 mmol) and DMSO (5 ml). The RM was stirred at RT for 1 h, solid NaBH₃CN (176 mg, 2.8 mmol) and MeOH (2 ml) were added and the RM was stirred at RT for 0.5 h. The mixture was concentrated and the residue was purified by reversed phase chromatography on a Biotage Agela C18 column (120 g, spherical 20-35 μm, 100 Å) eluting with ACN (from 5% to 95%) in an aq. solution of NH₄HCO₃ (0.1%), yielding the title compound as a solid (330 mg).

Method H: Rt=2.10 min; [M+H]⁺=592.

### Step 2: 4-(3,5-dimethoxy-4-((4-(piperidin-4-ylmethyl)piperazin-1-yl)methyl)phenyl)-2-methyl-2,7-naphthyridin-1(2H)-one

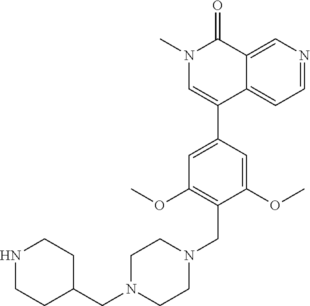 [SMILES: COc1cc(-c2cn(C)c(=O)c3cnccc23)cc(OC)c1CN1CCN(CC2CCNCC2)CC1]

To a 25 ml round bottom flask were added tert-butyl 4-((4-(2,6-dimethoxy-4-(2-methyl-1-oxo-1,2-dihydro-2,7-naphthyridin-4-yl)benzyl)piperazin-1-yl)methyl)piperidine-1-carboxylate (330 mg, 0.56 mmol), DCM (20 ml) and a solution of HCl (4 M) in 1,4-dioxane (2 ml). The RM was stirred at RT for 1 h and evaporated to dryness, yielding the corresponding hydrochloride salt of the title compound as a solid (350 mg), which was used for the next step without further purification.

Method A: Rt=1.08 min; [M+H]⁺=492.

### Step 3:1-(5-(4-((4-(2,6-dimethoxy-4-(2-methyl-1-oxo-1,2-dihydro-2,7-naphthyridin-4-yl)benzyl)piperazin-1-yl)methyl)piperidine-1-carbonyl)-2-methoxyphenyl)dihydropyrimidine-2,4(1H,3H)-dione

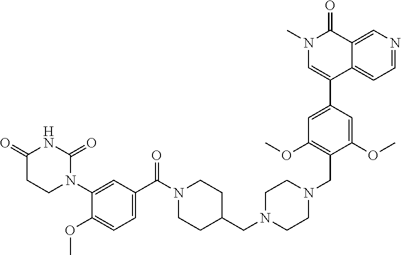 [SMILES: COc1ccc(C(=O)N2CCC(CN3CCN(Cc4c(OC)cc(-c5cn(C)c(=O)c6cnccc56)cc4OC)CC3)CC2)cc1N1CCC(=O)NC1=O]

To a 25 ml round bottom flask were added 4-(3,5-dimethoxy-4-((4-(piperidin-4-ylmethyl)piperazin-1-yl)methyl)phenyl)-2-methyl-2,7-naphthyridin-1(2H)-one hydrochloride (350 mg, 0.55 mmol), perfluorophenyl 3-(2,4-dioxotetrahydropyrimidin-1(2H)-yl)-4-methoxybenzoate (intermediate 26, 236 mg, 0.55 mmol), DIEA (284 mg, 2.2 mmol) and DMF (4 ml). The RM was stirred at RT for 1 h, the solvent was removed and the residue was purified by preparative HPLC on an XBridge C18 column (250×21.2 mm, 10 μm) eluting with ACN (from 5% to 80%) in an aq. solution of NH₄HCO₃ (10 mM), yielding the title compound as a solid (110 mg).

Method H: Rt=1.58 min; [M+H]⁺=738.

¹H NMR (500 MHz, DMSO-d₆) δ 10.34 (s, 1H), 9.44 (s, 1H), 8.72 (d, J=5.7 Hz, 1H), 7.88 (s, 1H), 7.58 (d, J=5.7 Hz, 1H), 7.35 (dd, J=8.4, 1.9 Hz, 1H), 7.31 (d, J=2.0 Hz, 1H), 7.15 (d, J=8.7 Hz, 1H), 6.72 (s, 2H), 3.84 (s, 3H), 3.80 (s, 6H), 3.60-3.58 (s, 5H), 3.55 (s, 2H), 2.68 (t, J=6.1 Hz, 2H), 2.49-2.18 (m, 12H), 2.10 (d, J=7.6 Hz, 2H), 1.76-1.60 (m, 3H), 1.07-0.99 (m, 2H).

### Compound A25: 1-(4-(2-(4-((1-(2,6-Dimethoxy-4-(2-methyl-1-oxo-1,2-dihydro-2,7-naphthyridin-4-yl)phenethyl)piperidin-4-yl)oxy)piperidin-1-yl)-2-oxoethoxy)phenyl)dihydropyrimidine-2,4(1H,3H)-dione

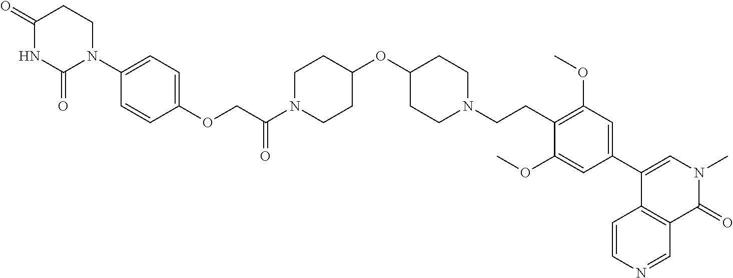 [SMILES: COc1cc(-c2cn(C)c(=O)c3cnccc23)cc(OC)c1CCN1CCC(OC2CCN(C(=O)COc3ccc(N4CCC(=O)NC4=O)cc3)CC2)CC1]

To a 50 mL round bottom flask were added 1-(4-(2-oxo-2-(4-(piperidin-4-yloxy)piperidin-1-yl)ethoxy)phenyl)dihydropyrimidine-2,4(1H,3H)-dione hydrochloride (intermediate 30, 100 mg, 0.2 mmol), 2-(2,6-dimethoxy-4-(2-methyl-1-oxo-1,2-dihydro-2,7-naphthyridin-4-yl)phenyl)acetaldehyde (intermediate 14, 81 mg, 0.24 mmol), K₂CO₃ (56 mg, 0.4 mmol) and DMSO (2 ml) and the RM was stirred at RT for 30 min. Solid NaBH₃CN (51 mg, 0.8 mmol) and MeOH (0.5 ml) were added and the RM was stirred at RT overnight. The mixture was concentrated and the residue was purified by chromatography on a Biotage Agela C18 column (40 g, spherical 20-35 μm, 100 Å) eluting with ACN (from 10% to 90%) in an aq solution of NH₄HCO₃ (0.1%), yielding the title compound as a solid (25 mg).

Method G: Rt=1.62 min; [M+H]⁺=753.

¹H NMR (500 MHz, DMSO-d₆) δ 10.31 (s, 1H), 9.44 (s, 1H), 8.71 (d, J=5.6 Hz, 1H), 7.84 (s, 1H), 7.55 (d, J=5.6 Hz, 1H), 7.22 (d, J=8.9 Hz, 2H), 6.70 (s, 2H), 4.82 (s, 2H), 3.88-3.78 (m, 7H), 3.74-3.64 (m, 4H), 3.59 (s, 3H), 3.49-3.40 (m, 2H), 3.26-3.17 (m, 1H), 3.15-3.06 (m, 1H), 2.94-2.65 (m, 6H), 2.42-2.25 (m, 3H), 1.94-1.74 (m, 4H), 1.51-1.30 (m, 6H).

### Compound A26: 3-(5-(4-((1-(2,5-Dimethoxy-4-(2-methyl-1-oxo-1,2-dihydro-2,7-naphthyridin-4-yl)benzyl)piperidin-4-yl)oxy)piperidine-1-carbonyl)-2-methoxyphenyl)piperidine-2,6-dione

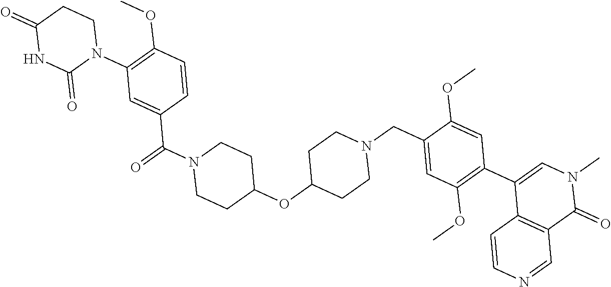 [SMILES: COc1cc(-c2cn(C)c(=O)c3cnccc23)c(OC)cc1CN1CCC(OC2CCN(C(=O)c3ccc(OC)c(N4CCC(=O)NC4=O)c3)CC2)CC1]

### Step 1: tert-butyl 4-((1-(2,5-dimethoxy-4-(2-methyl-1-oxo-1,2-dihydro-2,7-naphthyridin-4-yl)benzyl)piperidin-4-yl)oxy)piperidine-1-carboxylate

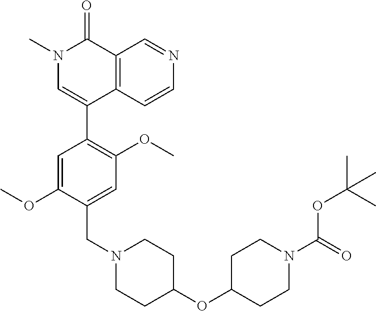 [SMILES: COc1cc(-c2cn(C)c(=O)c3cnccc23)c(OC)cc1CN1CCC(OC2CCN(C(=O)OC(C)(C)C)CC2)CC1]

To a 50 ml round bottom flask were added 2,5-dimethoxy-4-(2-methyl-1-oxo-1,2-dihydro-2,7-naphthyridin-4-yl)benzaldehyde (intermediate 12, 500 mg, 1.54 mmol), tert-butyl 4-(piperidin-4-yloxy)piperidine-1-carboxylate (intermediate 1, 438 mg, 1.54 mmol), a solution of ZnCl₂ (1 M) in THE (2.31 ml, 2.31 mmol) and DMSO (5 ml). The RM was stirred at RT for 3 h, solid NaBH₃CN (145 mg, 2.3 mmol) and MeOH (1 ml) were added and the RM was stirred at RT for 16 h. The mixture was concentrated and the residue was purified by reversed phase chromatography on a Biotage Agela C18 column (120 g, spherical 20-35 μm, 100 Å) eluting with ACN (from 5% to 95%) in an aq. solution of NH₄HCO₃ (0.1%), yielding the title compound as a solid (600 mg).

Method A: Rt=1.18 min; [M+H]⁺ 592.

### Step 2: 4-(2,5-dimethoxy-4-((4-(piperidin-4-yloxy)piperidin-1-yl)methyl)phenyl)-2-methyl-2,7-naphthyridin-1(2H)-one

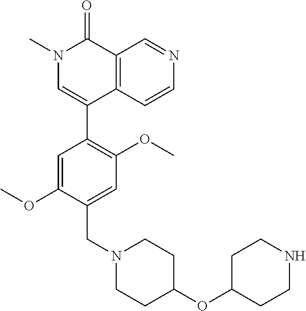 [SMILES: COc1cc(-c2cn(C)c(=O)c3cnccc23)c(OC)cc1CN1CCC(OC2CCNCC2)CC1]

To a 250 ml round bottom flask were added tert-butyl 4-((1-(2,5-dimethoxy-4-(2-methyl-1-oxo-1,2-dihydro-2,7-naphthyridin-4-yl)benzyl)piperidin-4-yl)oxy)piperidine-1-carboxylate (600 mg, 1.01 mmol), a solution of HCl (4 M) in 1,4-dioxane (10 ml), MeOH (5 ml) and 1,4-dioxane (15 ml). The RM was stirred at RT for 6 h and concentrated, yielding the tile compound as a solid, which was directly used for the next step without further purification.

Method A: Rt=0.32 min; [M+H]⁺ 429.

### Step 3: 3-(5-(4-((1-(2,5-dimethoxy-4-(2-methyl-1-oxo-1,2-dihydro-2,7-naphthyridin-4-yl)benzyl)piperidin-4-yl)oxy)piperidine-1-carbonyl)-2-methoxyphenyl)piperidine-2,6-dione

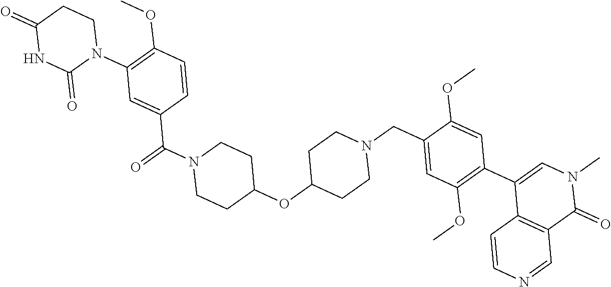 [SMILES: COc1cc(-c2cn(C)c(=O)c3cnccc23)c(OC)cc1CN1CCC(OC2CCN(C(=O)c3ccc(OC)c(N4CCC(=O)NC4=O)c3)CC2)CC1]

To a 50 ml round bottom flask was added 4-(2,5-dimethoxy-4-((4-(piperidin-4-yloxy)piperidin-1-yl)methyl)phenyl)-2-methyl-2,7-naphthyridin-1(2H)-one (1.01 mmol), perfluorophenyl 3-(2,4-dioxotetrahydropyrimidin-1(2H)-yl)-4-methoxybenzoate (intermediate 26, 912 mg, 2.12 mmol), TEA (1070 mg, 10.6 mmol), and DMF (10 ml). The RM was stirred at RT for 3 h, the mixture was concentrated and the residue was purified by preparative HPLC on an XBridge C18 column (250×21.2 mm, 10 μm) eluting with ACN (from 5% to 95%) in an aq. solution of NH₄HCO₃ (10 mM), yielding the title compound as a solid (800 mg).

Method H: Rt=1.57 min; [M+H]⁺ 738.

¹H NMR (500 MHz, DMSO-d₆) δ 10.34 (s, 1H), 9.41 (s, 1H), 8.64 (d, J=5.6 Hz, 1H), 7.74 (s, 1H), 7.41-7.31 (m, 2H), 7.15 (d, J=8.6 Hz, 2H), 7.03 (d, J=5.6 Hz, 1H), 6.94 (s, 1H), 3.84 (s, 4H), 3.75 (s, 3H), 3.71 (s, 2H), 3.64 (s, 3H), 3.62-3.43 (m, 8H), 3.34-3.21 (m, 3H), 2.80 (s, 2H), 2.68 (t, J=6.2 Hz, 2H), 2.42-2.06 (m, 2H), 1.86 (s, 4H), 1.48 (d, J=45.4 Hz, 4H).

Compound A27: (E)-1-(5-(4-((1-(3-(2,5-dimethoxy-4-(2-methyl-1-oxo-1,2-dihydro-2,7-naphthyridin-4-yl)phenyl)acryloyl)piperidin-4-yl)oxy)piperidine-1-carbonyl)-2-methoxyphenyl)dihydropyrimidine-2,4(1H,3H)-dione

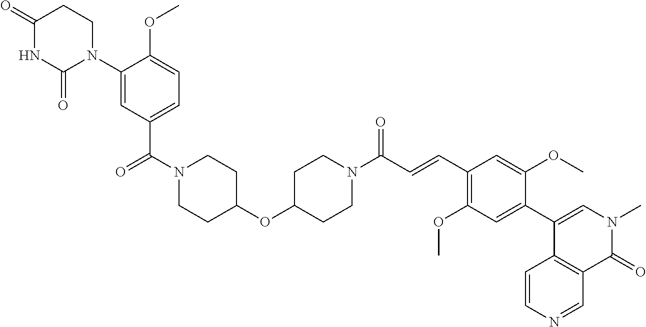 [SMILES: COc1cc(-c2cn(C)c(=O)c3cnccc23)c(OC)cc1/C=C/C(=O)N1CCC(OC2CCN(C(=O)c3ccc(OC)c(N4CCC(=O)NC4=O)c3)CC2)CC1]

### Step 1: methyl(E)-3-(2,5-dimethoxy-4-(4,4,5,5-tetramethyl-1,3,2-dioxaborolan-2-yl)phenyl)acrylate

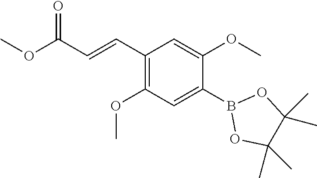

To a 100 ml round bottom flask were added 2,5-dimethoxy-4-(4,4,5,5-tetramethyl-1,3,2-dioxaborolan-2-yl)benzaldehyde (1.5 g, 5.13 mmol) and THE (10 ml). Solid NaH (60% dispersion in mineral oil, 820 mg, 20.54 mmol) was added portionwise at 0° C. and the RM was stirred at RT for 30 min, cooled again to 0° C. and a solution of methyl 2-(dimethoxyphosphoryl)acetate (2.8 g, 15.4 mmol) in THE (10 mL) was added dropwise. The RM was stirred at RT for 16 h, cooled to 0° C. and quenched with an aq. sat. solution of NH₄Cl (20 ml). The mixture was extracted with EA (2×50 ml), the combined organic phases were washed with brine (20 ml), dried over Na₂SO₄, and the residue was purified by chromatography on silica gel eluting with EtOAc (from 0% to 10%) in PE, yielding the title compound as a solid (1.5 g).

Method E: Rt=2.112 min; [M+H]⁺=349.

### Step 2: methyl (E)-3-(2,5-dimethoxy-4-(2-methyl-1-oxo-1,2-dihydro-2,7-naphthyridin-4-yl)phenyl)acrylate

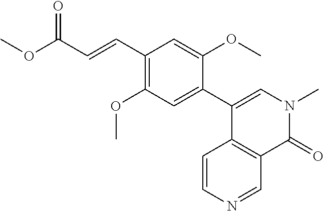 [SMILES: COC(=O)/C=C/c1cc(OC)c(-c2cn(C)c(=O)c3cnccc23)cc1OC]

To a 100 ml round bottom flask were added under an argon atmosphere methyl (E)-3-(2,5-dimethoxy-4-(4,4,5,5-tetramethyl-1,3,2-dioxaborolan-2-yl)phenyl)acrylate (200 mg, 0.57 mmol), 4-bromo-2-methyl-2,7-naphthyridin-1(2H)-one (intermediate 5, 137 mg, 0.57 mmol), Na₂CO₃ (183 mg, 1.72 mmol), 1,4-dioxane (15 ml), water (3 ml) and PdCl₂(dppf) (21 mg, 0.03 mmol). The RM was stirred at 80° C. for 16 h, added into EtOAc (200 ml), the organic phase was washed with brine (2×20 ml), filtered, the filtrate was concentrated and the residue was purified by chromatography on silica gel eluting with EtOAc (from 30% to 100%) in PE, yielding the title compound as a solid (200 mg).

Method G: Rt=1.742 min; [M+H]⁺=381.

### Step 3: (E)-3-(2,5-dimethoxy-4-(2-methyl-1-oxo-1,2-dihydro-2,7-naphthyridin-4-yl)phenyl)acrylic Acid

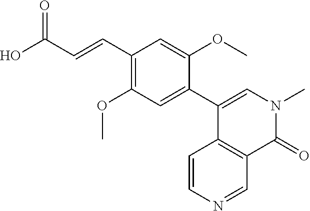 [SMILES: COc1cc(-c2cn(C)c(=O)c3cnccc23)c(OC)cc1/C=C/C(=O)O]

To a 100 ml round bottom flask were added methyl (E)-3-(2,5-dimethoxy-4-(2-methyl-1-oxo-1,2-dihydro-2,7-naphthyridin-4-yl)phenyl)acrylate (100 mg, 0.26 mmol), LiOH*H₂O (33 mg, 0.78 mmol), water (1 ml), THE (3 ml) and MeOH (1 ml) and the RM was stirred at 60° C. for 1 h. The mixture was concentrated, water (2 ml) was added and the pH of the mixture was adjusted to 4 by the addition of an aq. solution of HCl (1 M, 1.0 ml). The mixture was filtered, the solids were washed with water (3 ml) and dried, yielding the title compound as a solid (95 mg).

Method G: Rt=1.258 min; [M+H]⁺=367.

### Step 4: (E)-1-(5-(4-((1-(3-(2,5-dimethoxy-4-(2-methyl-1-oxo-1,2-dihydro-2,7-naphthyridin-4-yl)phenyl)acryloyl)piperidin-4-yl)oxy)piperidine-1-carbonyl)-2-methoxyphenyl)dihydropyrimidine-2,4(1H,3H)-dione

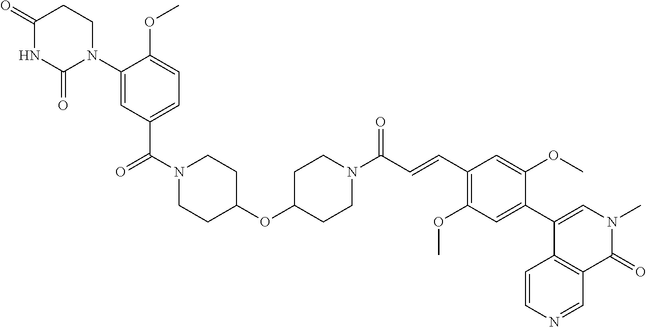 [SMILES: COc1cc(-c2cn(C)c(=O)c3cnccc23)c(OC)cc1/C=C/C(=O)N1CCC(OC2CCN(C(=O)c3ccc(OC)c(N4CCC(=O)NC4=O)c3)CC2)CC1]

To a 50 ml round bottom flask were added (E)-3-(2,5-dimethoxy-4-(2-methyl-1-oxo-1,2-dihydro-2,7-naphthyridin-4-yl)phenyl)acrylic acid (50 mg, 0.14 mmol), 1-(2-methoxy-5-(4-(piperidin-4-yloxy)piperidine-1-carbonyl)phenyl)dihydropyrimidine-2,4(1H,3H)-dione hydrochloride (intermediate 29, 64 mg, 0.14 mmol), DIPEA (55 mg, 0.42 mmol) and DMF (3 ml) at RT. The RM was stirred at RT for 10 min, solid HATU (65 mg, 0.17 mmol) was added and stirring was continued at RT for 2 h. The mixture was concentrated and the residue was purified by preparative HPLC on an XBridge C18 column (21.2×250 mm, 10 μm) eluting with ACN (from 10% to 90%) in an aq. solution of NH₄HCO₃ (10 mM), yielding the title compound as a solid (60 mg).

Method G: Rt=1.63 min; [M+H]⁺=779.

¹H NMR (500 MHz, DMSO-d₆) δ 10.35 (s, 1H), 9.41 (s, 1H), 8.65 (d, J=5.6 Hz, 1H), 7.85 (d, J=5.6 Hz, 1H), 7.78 (s, 1H), 7.53 (s, 1H), 7.41-7.31 (m, 3H), 7.16 (d, J=8.6 Hz, 1H), 7.08-7.00 (m, 2H), 4.00 (s, 3H), 3.84 (d, J=6.0 Hz, 6H), 3.78-3.73 (m, 2H), 3.72 (s, 3H), 3.61-3.57 (m, 5H), 3.44 (s, 2H), 3.26-3.18 (m, 3H), 2.72-2.62 (m, 2H), 1.85 (s, 4H), 1.45 (s, 4H).

### Compound A28: 1-(2-Chloro-5-(4-((1-(3-(2,6-dimethoxy-4-(2-methyl-1-oxo-1,2-dihydro-2,7-naphthyridin-4-yl)phenyl)propyl)piperidin-4-yl)oxy)piperidine-1-carbonyl)phenyl)dihydropyrimidine-2,4(1H,3H)-dione

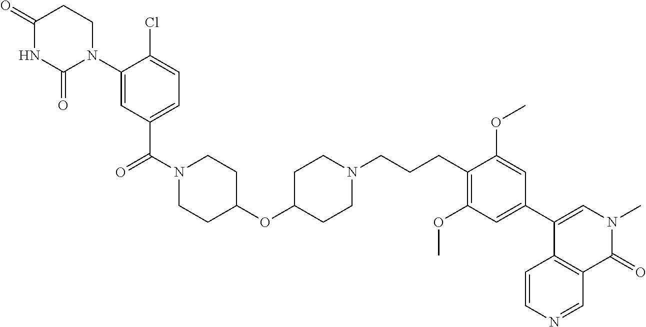 [SMILES: COc1cc(-c2cn(C)c(=O)c3cnccc23)cc(OC)c1CCCN1CCC(OC2CCN(C(=O)c3ccc(Cl)c(N4CCC(=O)NC4=O)c3)CC2)CC1]

To a 50 ml round bottom flask were added 3-(2,6-dimethoxy-4-(2-methyl-1-oxo-1,2-dihydro-2,7-naphthyridin-4-yl)phenyl)propanal (intermediate 13, 180 mg, 0.51 mmol), 1-(2-chloro-5-(4-(piperidin-4-yloxy)piperidine-1-carbonyl)phenyl)dihydropyrimidine-2,4(1H,3H)-dione (intermediate 28, 243 mg, 0.56 mmol), K₂CO₃ (140 mg, 2.04 mmol) and DMSO (2 ml). The RM was stirred at RT for 10 min, a solution of ZnCl₂ (1 M) in THE (0.66 ml, 0.66 mmol) was added and the RM was stirred at RT for 30 min. Solid NaBH₃CN (129 mg, 2.04 mmol) and MeOH (0.5 ml) were added and the RM was stirred at RT for 16 h. The mixture was filtered, the filtrate concentrated and the residue purified by preparative HPLC on an Xtimate C18 column (250×21.2 mm, 10 μm) eluting with ACN (from 5% to 80%) in an aq. solution of NH₄HCO₃ (10 mM), yielding the title compound as a solid (116 mg).

Method G: Rt=1.73 min; [M+H]⁺=771.

¹H NMR (500 MHz, DMSO-d₆) δ 10.51 (s, 1H), 9.44 (s, 1H), 8.71 (d, J=5.6 Hz, 1H), 7.84 (s, 1H), 7.63 (d, J=8.2 Hz, 1H), 7.60-7.52 (m, 2H), 7.40 (dd, J=8.2, 1.9 Hz, 1H), 6.69 (s, 2H), 4.05-3.89 (m, 1H), 3.80 (s, 6H), 3.77-3.66 (m, 2H), 3.65-3.56 (m, 4H), 3.54-3.37 (m, 2H), 3.29-3.09 (m, 2H), 2.82-2.63 (m, 4H), 2.62-2.55 (m, 2H), 2.28 (t, J=7.3 Hz, 2H), 2.07-1.91 (m, 2H), 1.90-1.67 (m, 4H), 1.64-1.51 (m, 2H), 1.51-1.32 (m, 4H).

### Compound A29: 1-(5-(4-((1-(2,5-Dimethoxy-4-(2-methyl-1-oxo-1,2-dihydro-2,7-naphthyridin-4-yl)benzyl)piperidin-4-yl)oxy)piperidine-1-carbonyl)-2-methylphenyl)dihydropyrimidine-2,4(1H,3H)-dione

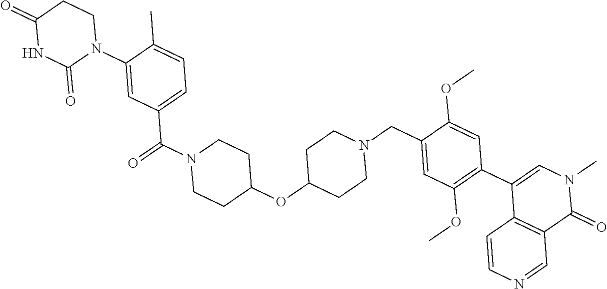 [SMILES: COc1cc(-c2cn(C)c(=O)c3cnccc23)c(OC)cc1CN1CCC(OC2CCN(C(=O)c3ccc(C)c(N4CCC(=O)NC4=O)c3)CC2)CC1]

### Step 1: tert-butyl 4-((1-(2,5-dimethoxy-4-(2-methyl-1-oxo-1,2-dihydro-2,7-naphthyridin-4-yl)benzyl)piperidin-4-yl)oxy)piperidine-1-carboxylate

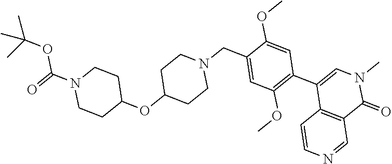 [SMILES: COc1cc(-c2cn(C)c(=O)c3cnccc23)c(OC)cc1CN1CCC(OC2CCN(C(=O)OC(C)(C)C)CC2)CC1]

To a 50 ml round bottom flask were added 2,5-dimethoxy-4-(2-methyl-1-oxo-1,2,3,4-tetrahydro-2,7-naphthyridin-4-yl)benzaldehyde (intermediate 12, 1 g, 3.09 mmol), tert-butyl 4-(piperidin-4-yloxy)piperidine-1-carboxylate (intermediate 1, 877 mg, 3.09 mmol), a solution of ZnCl₂ (1 M) in THE (4 ml, 4 mmol) and DMSO (6 ml). The RM was stirred at RT for 30 min, solid NaBH₃CN (799 mg, 12.36 mmol) and MeOH (1 ml) were added and the RM was stirred at RT for 16 h. The mixture was filtered, the filtrate was added into water (15 ml), the aq. phase was extracted with EtOAc (4×20 ml) and the combined organic phases were dried over Na₂SO₄ and the residue was purified by chromatography on silica gel eluting with MeOH (from 0% to 10%) in DCM, yielding the title compound as a solid (780 mg).

Method G: Rt=2.02 min; [M+H]⁺=593.

### Step 2: 4-(2,5-dimethoxy-4-((4-(piperidin-4-yloxy)piperidin-1-yl)methyl)phenyl)-2-methyl-2,7-naphthyridin-1(2H)-one

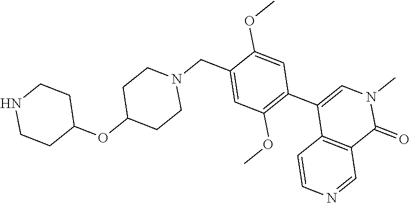 [SMILES: COc1cc(-c2cn(C)c(=O)c3cnccc23)c(OC)cc1CN1CCC(OC2CCNCC2)CC1]

To a 50 ml round bottom flask were added tert-butyl 4-((1-(2,5-dimethoxy-4-(2-methyl-1-oxo-1,2-dihydro-2,7-naphthyridin-4-yl)benzyl)piperidin-4-yl)oxy)piperidine-1-carboxylate (150 mg, 0.25 mmol), DCM (4 ml) and a solution of HCl (4 M) in 1,4-dioxane (2 ml). The RM was stirred at RT for 1 h and the mixture was concentrated, yielding the corresponding hydrochloride salt of the title compound as a solid (160 mg), which was used for the next step without further purification.

Method G: Rt=1.68 min; [M+H]⁺=493.

### Step 3:1-(5-(4-((1-(2,5-dimethoxy-4-(2-methyl-1-oxo-1,2-dihydro-2,7-naphthyridin-4-yl)benzyl)piperidin-4-yl)oxy)piperidine-1-carbonyl)-2-methylphenyl)dihydropyrimidine-2,4(1H,3H)-dione

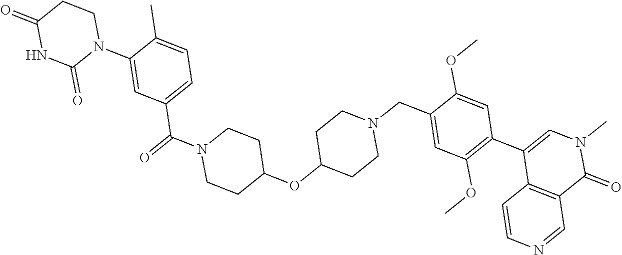 [SMILES: COc1cc(-c2cn(C)c(=O)c3cnccc23)c(OC)cc1CN1CCC(OC2CCN(C(=O)c3ccc(C)c(N4CCC(=O)NC4=O)c3)CC2)CC1]

To a 50 ml round bottom flask were added 4-(2,5-dimethoxy-4-((4-(piperidin-4-yloxy)piperidin-1-yl)methyl)phenyl)-2-methyl-2,7-naphthyridin-1(2H)-one hydrochloride (160 mg, 0.25 mmol), 3-(2,4-dioxotetrahydropyrimidin-1(2H)-yl)-4-methylbenzoic acid (intermediate 23, 62 mg, 0.25 mmol), DMF (2 ml), DIEA (161 mg, 1.25 mmol) and HATU (114 mg, 0.3 mmol). The RM was stirred at RT for 1 h, the mixture was concentrated and the residue was purified by preparative HPLC on an Xtimate C18 column (250×21.2 mm, 10 μm) eluting with ACN (from 5% to 67%) in an aq. solution of NH₄HCO₃ (10 mM), yielding the title compound as a solid (121 mg).

Method G: Rt=1.63 min; [M+H]⁺=723.

¹H NMR (500 MHz, DMSO-d₆) δ 10.38 (s, 1H), 9.40 (s, 1H), 8.64 (d, J=5.6 Hz, 1H), 7.74 (s, 1H), 7.38-7.31 (m, 2H), 7.26 (d, J=7.7 Hz, 1H), 7.14 (s, 1H), 7.03 (d, J=5.6 Hz, 1H), 6.91 (s, 1H), 4.09-3.87 (m, 1H), 3.86-3.77 (m, 1H), 3.76-3.67 (m, 4H), 3.63 (s, 3H), 3.61-3.42 (m, 8H), 3.29-3.11 (m, 2H), 2.89-2.63 (m, 4H), 2.30-2.06 (m, 5H), 1.96-1.66 (m, 4H), 1.57-1.35 (m, 4H).

### Compound A30: 1-(2-Chloro-5-(4-((1-(2,5-dimethoxy-4-(2-methyl-1-oxo-1,2-dihydro-2,7-naphthyridin-4-yl)benzyl)piperidin-4-yl)oxy)piperidine-1-carbonyl)phenyl)dihydropyrimidine-2,4(1H,3H)-dione

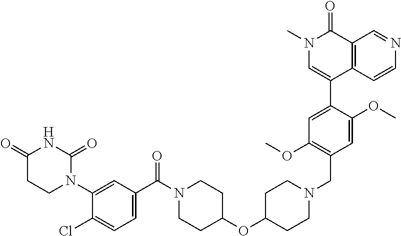 [SMILES: COc1cc(-c2cn(C)c(=O)c3cnccc23)c(OC)cc1CN1CCC(OC2CCN(C(=O)c3ccc(Cl)c(N4CCC(=O)NC4=O)c3)CC2)CC1]

To a 50 ml round bottom flask were added 1-(2-chloro-5-(4-(piperidin-4-yloxy)piperidine-1-carbonyl)phenyl)dihydropyrimidine-2,4(1H,3H)-dione hydrochloride (intermediate 28 170 mg, 0.36 mmol), 2,5-dimethoxy-4-(2-methyl-1-oxo-1,2-dihydro-2,7-naphthyridin-4-yl)benzaldehyde (intermediate 12, 126 mg, 0.39 mmol), K₂CO₃ (108 mg, 0.78 mmol), a solution of ZnCl₂ (1 M) in THE (0.51 ml, 0.51 mmol) and DMSO (2 ml). The RM was stirred at RT for 0.5 h, solid NaBH₃CN (98 mg, 1.56 mmol) was added and the RM was stirred at RT for 16 h. The mixture was added into water (20 ml) and filtered. The solids were washed with water (2×10 ml), dried and purified by chromatography on silica gel eluting with MeOH (from 5% to 15%) in DCM containing 0.1% TEA, yielding the title compound as a solid (45 mg).

Method G: Rt=1.65 min; [M+H]⁺ 372.

¹H NMR (500 MHz, DMSO-d₆) δ 10.52 (s, 1H), 9.40 (s, 1H), 8.64 (d, J=5.6 Hz, 1H), 7.74 (s, 1H), 7.64 (d, J=8.2 Hz, 1H), 7.58 (s, 1H), 7.40 (dd, J=8.1, 1.7 Hz, 1H), 7.14 (s, 1H), 7.03 (d, J=5.6 Hz, 1H), 6.92 (s, 1H), 4.08-3.90 (m, 1H), 3.80-3.69 (m, 5H), 3.68-3.61 (m, 4H), 3.57 (s, 3H), 3.53-3.44 (m, 4H), 3.28-3.10 (m, 2H), 2.83-2.67 (m, 4H), 2.17 (t, J=10.7 Hz, 2H), 1.99-1.68 (m, 4H), 1.58-1.36 (m, 4H).

### Compound A31: (E)-1-(5-(4-((1-(3-(2,6-dimethoxy-4-(2-methyl-1-oxo-1,2-dihydro-2,7-naphthyridin-4-yl)phenyl)acryloyl)piperidin-4-yl)oxy)piperidine-1-carbonyl)-2-methoxyphenyl)dihydropyrimidine-2,4(1H,3H)-dione

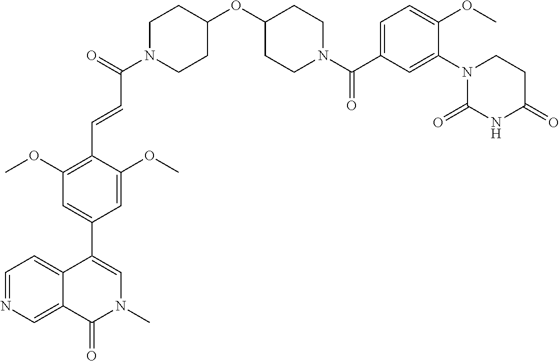 [SMILES: COc1ccc(C(=O)N2CCC(OC3CCN(C(=O)/C=C/c4c(OC)cc(-c5cn(C)c(=O)c6cnccc56)cc4OC)CC3)CC2)cc1N1CCC(=O)NC1=O]

### Step 1: methyl (E)-3-(2,6-dimethoxy-4-(2-methyl-1-oxo-1,2-dihydro-2,7-naphthyridin-4-yl)phenyl)acrylate

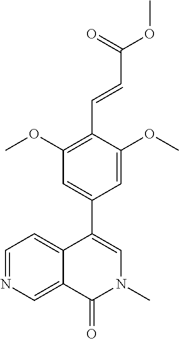 [SMILES: COC(=O)/C=C/c1c(OC)cc(-c2cn(C)c(=O)c3cnccc23)cc1OC]

To a 100 ml round bottom flask were added methyl 2-(dimethoxyphosphoryl)acetate (168 mg, 0.93 mmol), NaH (60% dispersion in mineral oil, 50 mg, 1.23 mmol) and THE (20 ml). The RM was stirred at RT for 10 min, solid 2,6-dimethoxy-4-(2-methyl-1-oxo-1,2-dihydro-2,7-naphthyridin-4-yl)benzaldehyde (intermediate 11, 200 mg, 0.617 mmol) was added and the RM was stirred at RT for 16 h. The mixture was filtered, the filtrate was added into water (10 ml), the mixture was extracted with EtOAc (4×50 ml) and the combined organic phases were dried over Na₂SO₄, concentrated and the residue was purified by chromatography on silica gel eluting with MeOH (from 10% to 40%) in DCM, yielding the title compound as a solid (201 mg).

Method J: Rt=1.30 min; [M+H]⁺=381.

### Step 2: (E)-3-(2,6-dimethoxy-4-(2-methyl-1-oxo-1,2-dihydro-2,7-naphthyridin-4-yl)phenyl)acrylic Acid

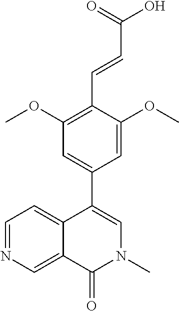 [SMILES: COc1cc(-c2cn(C)c(=O)c3cnccc23)cc(OC)c1/C=C/C(=O)O]

To a 100 ml round bottom flask were added methyl (E)-3-(2,6-dimethoxy-4-(2-methyl-1-oxo-1,2-dihydro-2,7-naphthyridin-4-yl)phenyl)acrylate (190 mg, 0.5 mmol), MeOH (10 ml) and THE (10 ml). A solution of NaOH (120 mg, 3 mmol) in water (10 ml) was added dropwised at 25° C. over 5 min and the RM was stirred at 25° C. for 2 h. Cold water (100 ml) was added and the pH of the mixture was adjusted to 4-5 by the addition of an aq. solution of HCl (3 M). The mixture was filtered and the solids were washed with cold water, yielding the title compound as a solid (145 mg).

Method J: Rt=0.82 min; [M+H]⁺=366.

### Step 3: (E)-1-(5-(4-((1-(3-(2,6-dimethoxy-4-(2-methyl-1-oxo-1,2-dihydro-2,7-naphthyridin-4-yl)phenyl)acryloyl)piperidin-4-yl)oxy)piperidine-1-carbonyl)-2-methoxyphenyl)dihydropyrimidine-2,4(1H,3H)-dione

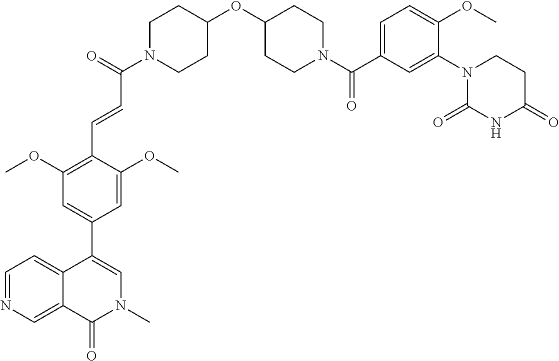 [SMILES: COc1ccc(C(=O)N2CCC(OC3CCN(C(=O)/C=C/c4c(OC)cc(-c5cn(C)c(=O)c6cnccc56)cc4OC)CC3)CC2)cc1N1CCC(=O)NC1=O]

To a 50 ml round bottom flask were added (E)-3-(2,6-dimethoxy-4-(2-methyl-1-oxo-1,2-dihydro-2,7-naphthyridin-4-yl)phenyl)acrylic acid (120 mg, 0.328 mmol), 1-(2-methoxy-5-(4-(piperidin-4-yloxy)piperidine-1-carbonyl)phenyl)dihydropyrimidine-2,4(1H,3H)-dione hydrochloride (intermediate 29, 153 mg, 0.328 mmol), DMF (4 ml), DIEA (211 mg, 1.64 mmol) and HATU (137 mg, 0.361 mmol). The RM was stirred at at 25° C. for 3 h, the mixture was concentrated and the residue was purified by preparative HPLC on an Xtimate C18 column (250×21.2 mm, 10 μm) eluting with ACN (from 5% to 60%) in an aq. solution of NH₄HCO₃ (10 mM), yielding the title compound as a solid (78 mg).

Method H: Rt=1.6 min; [M+H]⁺=779.

¹H NMR (500 MHz, DMSO-d₆) δ 10.35 (s, 1H), 9.45 (s, 1H), 8.73 (d, J=5.7 Hz, 1H), 7.93 (s, 1H), 7.85 (d, J=15.7, 1.8 Hz, 1H), 7.62 (d, J=5.7, 1H), 7.44-7.39 (m, 1H), 7.38 (d, J=3.8, 1H), 7.35 (d, J=2.1 Hz, 1H), 7.16 (d, J=8.6 Hz, 1H), 6.80 (s, 2H), 3.91 (s, 8H), 3.84 (s, 4H), 3.74 (s, 3H), 3.63-3.58 (m, 5H), 3.34 (s, 1H), 3.23 (s, 3H), 2.69 (d, J=5.9 Hz, 2H), 1.83 (s, 4H), 1.44 (s, 4H).

### Compound A32: 1-(5-(4-((1-(2,6-Dimethoxy-4-(2-methyl-1-oxo-1,2-dihydro-2,7-naphthyridin-4-yl)phenethyl)piperidin-4-yl)oxy)piperidine-1-carbonyl)-2-methoxyphenyl)dihydropyrimidine-2,4(1H,3H)-dione

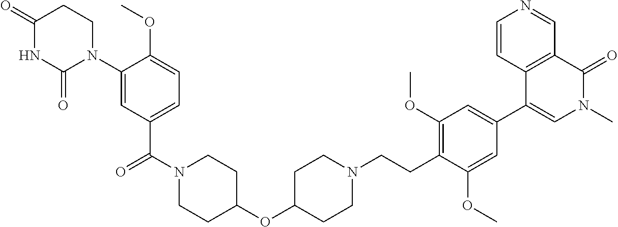 [SMILES: COc1ccc(C(=O)N2CCC(OC3CCN(CCc4c(OC)cc(-c5cn(C)c(=O)c6cnccc56)cc4OC)CC3)CC2)cc1N1CCC(=O)NC1=O]

To a 250 ml round bottom flask were added 2-(2,6-dimethoxy-4-(2-methyl-1-oxo-1,2-dihydro-2,7-naphthyridin-4-yl)phenyl)acetaldehyde (intermediate 14, 50 mg, 0.15 mmol), 1-(2-methoxy-5-(4-(piperidin-4-yloxy)piperidine-1-carbonyl)phenyl)dihydropyrimidine-2,4(1H,3H)-dione hydrochloride (intermediate 29, 100 mg, 0.16 mmol), K₂CO₃ (40 mg, 0.29 mmol), a solution of ZnCl₂ (1 M) in THE (0.19 ml, 0.19 mmol) and DMSO (2 ml). The RM was stirred at RT for 3 h, solid NaBH₃CN (75 mg, 1.2 mmol) and MeOH (1 ml) were added and the RM was stirred at RT for 16 h. The mixture was concentrated and the residue was purified by preparative HPLC on an XBridge C18 column (250×21.2 mm, 10 μm) eluting with ACN (from 5% to 80%) in an aq. solution of NH₄HCO₃ (10 mM), yielding the title compound as a solid (28 mg).

Method H: Rt=1.64 min; [M+H]⁺=753.

¹H NMR (500 MHz, DMSO-d₆) δ 10.33 (s, 1H), 9.44 (s, 1H), 8.71 (d, J=5.7 Hz, 1H), 7.84 (s, 1H), 7.55 (d, J=5.5 Hz, 1H), 7.38 (dd, J=8.5, 2.1 Hz, 1H), 7.34 (d, J=2.1 Hz, 1H), 7.15 (d, J=8.6, 0.6 Hz, 1H), 6.69 (s, 2H), 3.84 (s, 3H), 3.80 (s, 6H), 3.74-3.63 (m, 2H), 3.62-3.56 (m, 5H), 3.48-3.40 (m, 1H), 3.25-3.17 (m, 2H), 2.83-2.63 (m, 6H), 2.74-2.28 (m, 3H), 2.11 (t, J=8.1 Hz, 2H), 1.87-1.71 (m, 4H), 1.49-1.36 (m, 4H).

### Compound A33: 1-(5-(4-((1-(2,6-Dimethoxy-4-(2-methyl-1-oxo-1,2-dihydro-2,7-naphthyridin-4-yl)benzyl)piperidin-4-yl)oxy)piperidine-1-carbonyl)-2-methylphenyl)dihydropyrimidine-2,4(1H,3H)-dione

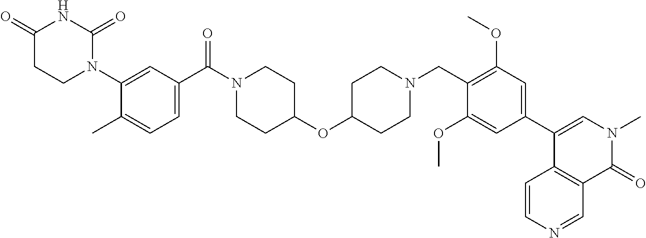 [SMILES: COc1cc(-c2cn(C)c(=O)c3cnccc23)cc(OC)c1CN1CCC(OC2CCN(C(=O)c3ccc(C)c(N4CCC(=O)NC4=O)c3)CC2)CC1]

### Step 1: tert-butyl 4-((1-(3-(2,4-dioxotetrahydropyrimidin-1(2H)-yl)-4-methylbenzoyl)piperidin-4-yl)oxy)piperidine-1-carboxylate

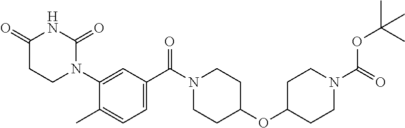 [SMILES: Cc1ccc(C(=O)N2CCC(OC3CCN(C(=O)OC(C)(C)C)CC3)CC2)cc1N1CCC(=O)NC1=O]

To a 50 ml round bottom flask was added tert-butyl 4-(piperidin-4-yloxy)piperidine-1-carboxylate (intermediate 1, 572 mg, 2 mmol), 3-(2,4-dioxotetrahydropyrimidin-1(2H)-yl)-4-methylbenzoic acid (intermediate 23, 500 mg, 2 mmol), HATU (836 mg, 2.2 mmol), DIEA (774 mg, 6 mmol) and DMF (10 ml). The RM was stirred at RT for 3 h, the mixture was concentrated and the residue was purified by preparative HPLC on an XBridge C18 column (250×21.2 mm, 10 μm) eluting with ACN (from 5% to 95%) in an aq. solution of NH₄HCO₃ (10 mM), yielding the title compound as a solid (920 mg).

Method J: Rt=1.25 min; [M+H]⁺=537.

### Step 2: 1-(2-methyl-5-(4-(piperidin-4-yloxy)piperidine-1-carbonyl)phenyl)dihydropyrimidine-2,4(1H,3H)-dione

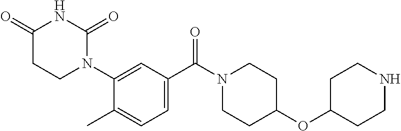 [SMILES: Cc1ccc(C(=O)N2CCC(OC3CCNCC3)CC2)cc1N1CCC(=O)NC1=O]

To a 50 ml round bottom flask were added tert-butyl 4-((1-(3-(2,4-dioxotetrahydropyrimidin-1(2H)-yl)-4-methylbenzoyl)piperidin-4-yl)oxy)piperidine-1-carboxylate (900 mg, 1.75 mmol), a solution of HCl (4 M) in 1,4-dioxane (10 ml) and MeOH (20 ml). The RM was stirred at RT for 4 h and evaporated to dryness, yielding the corresponding hydrochloride salt of the title compound as a solid (751 mg), which was used for the next step without further purification.

Method E: Rt=1.13 min; [M+H]⁺=415.

### Step 3: 1-(5-(4-((1-(2,6-dimethoxy-4-(2-methyl-1-oxo-1,2-dihydro-2,7-naphthyridin-4-yl)benzyl)piperidin-4-yl)oxy)piperidine-1-carbonyl)-2-methylphenyl)dihydropyrimidine-2,4(1H,3H)-dione

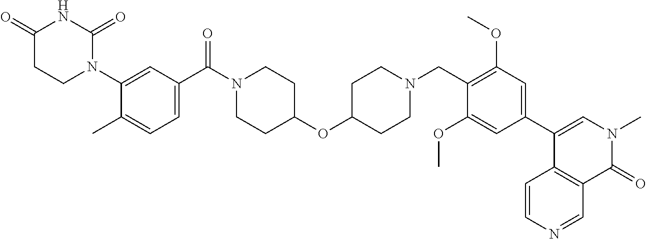 [SMILES: COc1cc(-c2cn(C)c(=O)c3cnccc23)cc(OC)c1CN1CCC(OC2CCN(C(=O)c3ccc(C)c(N4CCC(=O)NC4=O)c3)CC2)CC1]

To a 50 ml round bottom flask were added 1-(2-methyl-5-(4-(piperidin-4-yloxy)piperidine-1-carbonyl)phenyl)dihydropyrimidine-2,4(1H,3H)-dione hydrochloride (140 mg, 0.31 mmol), 2,6-dimethoxy-4-(2-methyl-1-oxo-1,2-dihydro-2,7-naphthyridin-4-yl)benzaldehyde (intermediate 11, 100 mg, 0.31 mmol), a solution of ZnCl₂ (1 M) in THF (0.47 ml, 0.47 mmol) and DMSO (3 ml). The RM was stirred at RT for 1 h, then solid NaBH₃CN (78 mg, 1.24 mmol) was added and stirring was continued at RT for 16 h. The mixture was concentrated and the residue was purified by reversed phase chromatography on a Biotage Agela C18 column (120 g, spherical 20-35 μm, 100 Å) eluting with ACN (from 0% to 40%) in an aq. solution of NH₄HCO₃ (0.1%), yielding the title compound as a solid (54 mg).

Method G: Rt=1.62 min; [M+H]⁺=723.

¹H NMR (500 MHz, DMSO-d₆) δ 10.38 (s, 1H), 9.44 (s, 1H), 8.72 (d, J=5.7 Hz, 1H), 7.88 (s, 1H), 7.58 (d, J=5.6 Hz, 1H), 7.34 (d, J=8.5 Hz, 2H), 7.25 (dd, J=7.7, 1.6 Hz, 1H), 6.72 (s, 2H), 3.96 (d, J=22.2 Hz, 1H), 3.80 (s, 7H), 3.66 (d, J=8.3 Hz, 1H), 3.60 (s, 3H), 3.57-3.45 (m, 4H), 3.38 (s, 1H), 3.18 (s, 2H), 2.84-2.64 (m, 4H), 2.21 (s, 3H), 2.14 (t, J=9.7 Hz, 2H), 1.74 (s, 4H), 1.37 (d, J=9.6 Hz, 4H).

### Compound A34: 1-(5-(4-(2-(4-(2,6-Dimethoxy-4-(2-methyl-1-oxo-1,2-dihydro-2,7-naphthyridin-4-yl)benzyl)piperazin-1-yl)ethoxy)piperidine-1-carbonyl)-2-methoxyphenyl)dihydropyrimidine-2,4(1H,3H)-dione

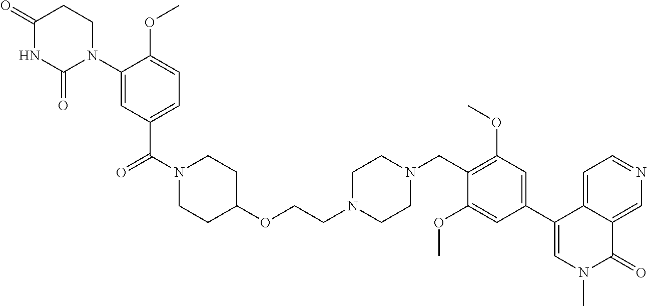 [SMILES: COc1ccc(C(=O)N2CCC(OCCN3CCN(Cc4c(OC)cc(-c5cn(C)c(=O)c6cnccc56)cc4OC)CC3)CC2)cc1N1CCC(=O)NC1=O]

### Step 1: 1-benzyl-4-(2,2-diethoxyethoxy)piperidine

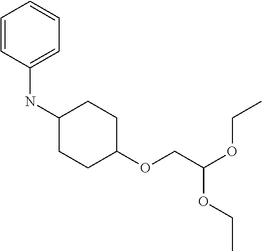 [SMILES: CCOC(COC1CCC(Nc2ccccc2)CC1)OCC]

To a 500 ml round bottom flask were added 1-benzylpiperidin-4-ol (5 g, 26.2 mmol) and THE (150 ml) and the solution was cooled to 0° C. Solid NaH (60% dispersion in mineral oil, 1.6 g, 39.3 mmol) was added portionwise, the RM was stirred at RT for 30 min and again cooled down to 0° C. 2-Bromo-1,1-diethoxyethane (6.6 g, 34 mmol) was added and the RM was stirred at RT for 16 h. Water (300 ml) was added and the mixture was extracted with EtOAc (3×150 ml), the combined organic phases were concentrated and the residue was purified by chromatography on silica gel eluting with MeOH (from 0% to 10%) in DCM, yielding the title compound as a oil (1.2 g).

Method J: Rt=1.43 min; [M+H]⁺=308.

### Step 2: 2-((1-benzylpiperidin-4-yl)oxy)acetaldehyde

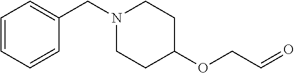 [SMILES: O=CCOC1CCN(Cc2ccccc2)CC1]

To a 100 ml round bottom flask were added 1-benzyl-4-(2,2-diethoxyethoxy)piperidine (900 mg, 2.93 mmol), THF (20 ml) and an aq. solution of HCI (3 M) in water (10 ml, 30 mmol). The RM was stirred at 25° C. for 4 h, extracted with EtOAc (3×30 ml) and the combined organic phases were dried over Na2SO4 and the residue was purified by chromatography on silica gel eluting with MeOH (from 5% to 1 %) in DCM, yielding the title compound as a oil (580 mg).

Method J: Rt32 1.02 min; [M+H]⁺252.

### Step 3: tert-butyl 4-(2-((1-benzylpipcridin-4-yl)oxy)ethyl)piperazinc-1-carboxylate

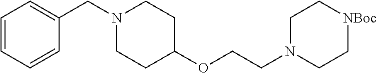 [SMILES: CC(C)(C)OC(=O)N1CCN(CCOC2CCN(Cc3ccccc3)CC2)CC1]

To a 50 mL round bottom flask were added 2-((1-benzylpiperidin-4-yl)oxy)acetaldehyde (550 mg, 2.2 mmol), tert-butyl piperazine-1-carboxylate (614 mg, 3.3 mmol), DMSO (10 ml) and a solution of ZnCl₂ (1 M) in THE (3.3 ml, 3.3 mmol). The RM was stirred at RT for 30 min, solid NaBH₃CN (1.10 g, 17.6 mmol) and MeOH (2 ml) were added. The RM was stirred at RT for 16 h, concentrated and the residue was purified by reversed phase chromatography on a Biotage Agela C18 column (40 g, spherical 20-35 μm, 100 Å) eluting with ACN (from 10% to 70%) in an aq. solution of NH₄HCO₃ (0.1%), yielding the title compound as an oil (180 mg).

Method J: Rt=1.48 min; [M+H]⁺=404.

### Step 4: tert-butyl 4-(2-(piperidin-4-yloxy)ethyl)piperazine-1-carboxylate

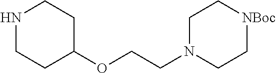 [SMILES: CC(C)(C)OC(=O)N1CCN(CCOC2CCNCC2)CC1]

To a 100 ml round bottom flask were added tert-butyl 4-(2-((1-benzylpiperidin-4-yl)oxy)ethyl)piperazine-1-carboxylate (180 mg, 0.45 mmol), Pd/C (10%, 60 mg) and methanol (20 ml). The RM was stirred under a H₂ atmosphere (1 bar) at RT for 2 h, the mixture was filtered and the filtrate was evaporated to dryness, yielding the title compound as a solid (110 mg).

Method F: Rt=1.61 min; [M+H]⁺=314.

### Step 5: tert-butyl 4-(2-((1-(3-(2,4-dioxotetrahydropyrimidin-1(2H)-yl)-4-methoxybenzoyl)piperidin-4-yl)oxy)ethyl)piperazine-1-carboxylate

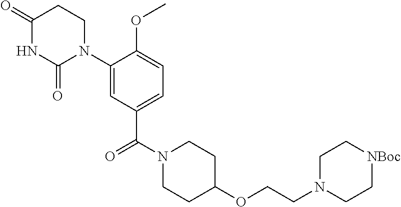 [SMILES: COc1ccc(C(=O)N2CCC(OCCN3CCN(C(=O)OC(C)(C)C)CC3)CC2)cc1N1CCC(=O)NC1=O]

To a 100 ml round bottom flask were added tert-butyl 4-(2-(piperidin-4-yloxy)ethyl)piperazine-1-carboxylate (110 mg, 0.35 mmol), perfluorophenyl 3-(2,4-dioxotetrahydropyrimidin-1(2H)-yl)-4-methoxybenzoate (intermediate 26, 151 mg 0.35 mmol), DIPEA (135 mg, 1.05 mmol) and DMF (4 ml) and the RM was stirred at RT for 2 h. The mixture was directly purified by preparative HPLC on an XBridge C18 column (21.2×250 mm, 10 μm) eluting with ACN (from 5% to 50%) in an aq. solution of NH₄HCO₃ (10 mM), yielding the title compound as a solid (135 mg).

Method G: Rt=1.64 min; [M+H]⁺=560.

### Step 6: 1-(2-methoxy-5-(4-(2-(piperazin-1-yl)ethoxy)piperidine-1-carbonyl)phenyl)dihydropyrimidine-2,4(1H,3H)-dione

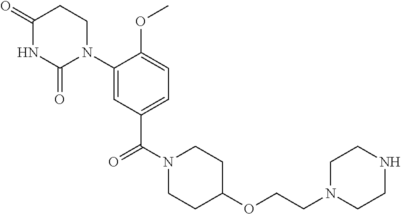 [SMILES: COc1ccc(C(=O)N2CCC(OCCN3CCNCC3)CC2)cc1N1CCC(=O)NC1=O]

To a 100 mL round bottom flask were added tert-butyl 4-(2-((1-(3-(2,4-dioxotetrahydropyrimidin-1(2H)-yl)-4-methoxybenzoyl)piperidin-4-yl)oxy)ethyl)piperazine-1-carboxylate (135 mg, 0.24 mmol), methanol (10 ml) and a solution of HCl (4 M) in 1,4-dioxane (5 ml). The RM was stirred at RT for 3 h and evaporated to dryness, yielding the corresponding hydrogen chloride salt of the title compound as a solid (110 mg), which was used without further purification for the next step.

Method E: Rt=1.08 min; [M+H]⁺=460.

### Step 7: 1-(5-(4-(2-(4-(2,6-dimethoxy-4-(2-methyl-1-oxo-1,2-dihydro-2,7-naphthyridin-4-yl)benzyl)piperazin-1-yl)ethoxy)piperidine-1-carbonyl)-2-methoxyphenyl)dihydropyrimidine-2,4(1H,3H)-dione

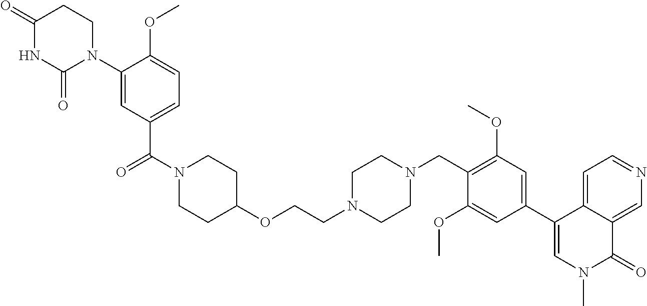 [SMILES: COc1ccc(C(=O)N2CCC(OCCN3CCN(Cc4c(OC)cc(-c5cn(C)c(=O)c6cnccc56)cc4OC)CC3)CC2)cc1N1CCC(=O)NC1=O]

To a 50 ml round bottom flask were added 1-(2-methoxy-5-(4-(2-(piperazin-1-yl)ethoxy)piperidine-1-carbonyl)phenyl)dihydropyrimidine-2,4(1H,3H)-dione hydrochloride (110 mg, 0.22 mmol), 2,6-dimethoxy-4-(2-methyl-1-oxo-1,2-dihydro-2,7-naphthyridin-4-yl)benzaldehyde (intermediate 11, 72 mg, 0.22 mmol), K₂CO₃ (61 mg, 0.44 mmol), a solution of ZnCl₂ (1 M) in THE (0.29 ml, 0.29 mmol) and DMSO (3 ml). The RM was stirred at RT for 30 min, solid NaBH₃CN (111 mg, 1.76 mmol) and methanol (1 ml) was added and the RM was stirred at RT for 16 h. The mixture was concentrated and the residue was purified by preparative HPLC on an XBridge C18 column (21.2×250 mm, 10 μm) eluting with ACN (from 10% to 90%) in an aq. solution of NH₄HCO₃ (10 mM), yielding of the title compound as a solid (34 mg).

Method G: Rt=1.59 min; [M+H]⁺=768.

¹H NMR (500 MHz, DMSO-d₆) δ 10.33 (s, 1H), 9.44 (s, 1H), 8.72 (d, J=5.7, 1H), 7.87 (s, 1H), 7.57 (d, J=5.7 Hz, 1H), 7.38 (dd, J=8.5, 2.1 Hz, 1H), 7.34 (d, J=2.1 Hz, 1H), 7.15 (d, J=8.6 Hz, 1H), 6.72 (s, 2H), 3.82 (d, J=20.5 Hz, 9H), 3.59 (d, J=8.8 Hz, 5H), 3.53 (d, J=16.3 Hz, 5H), 3.25 (d, J=39.7 Hz, 3H), 2.66 (d, J=20.2 Hz, 2H), 2.40 (d, J=32.5 Hz, 11H), 1.83 (s, 2H), 1.42 (s, 2H).

### Compound A35: 1-(4-(2-(4-((1-(2,6-Dimethoxy-4-(2-methyl-1-oxo-1,2-dihydro-2,7-naphthyridin-4-yl)benzyl)piperidin-4-yl)oxy)piperidin-1-yl)-2-oxoethoxy)phenyl)dihydropyrimidine-2,4(1H,3H)-dione

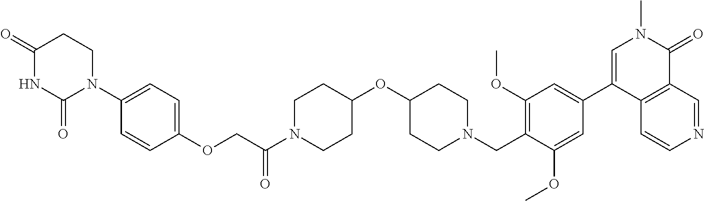 [SMILES: COc1cc(-c2cn(C)c(=O)c3cnccc23)cc(OC)c1CN1CCC(OC2CCN(C(=O)COc3ccc(N4CCC(=O)NC4=O)cc3)CC2)CC1]

To a 50 mL round bottom flask were added 1-(4-(2-oxo-2-(4-(piperidin-4-yloxy)piperidin-1-yl)ethoxy)phenyl)dihydropyrimidine-2,4(1H,3H)-dione hydrochloride (intermediate 30, 350 mg, 0.75 mmol), 2,6-dimethoxy-4-(2-methyl-1-oxo-1,2-dihydro-2,7-naphthyridin-4-yl)benzaldehyde (intermediate 11, 269 mg, 0.83 mmol), K₂CO₃ (311 mg, 2.25 mmol), DMSO (5 ml) and the RM was stirred at RT for 30 min. Solid NaBH₃CN (142 mg, 2.25 mmol) and MeOH (0.5 ml) were added and the RM was stirred at RT for 16 h. The mixture was concentrated and the residue was purified by reversed phase chromatography on a Biotage Agela C18 column (40 g, spherical 20-35 μm, 100 Å) eluting with ACN (from 10% to 90%) in an aq. solution of NH₄HCO₃ (0.1%) yielding the title compound as a solid (25 mg).

Method H: Rt=1.541 min; [M+H]⁺=739.

¹H NMR (500 MHz, DMSO-d₆) δ 10.31 (s, 1H), 9.44 (s, 1H), 8.72 (d, J=5.6 Hz, 1H), 7.84 (s, 1H), 7.58 (d, J=5.6 Hz, 1H), 7.22 (d, J=8.9 Hz, 2H), 6.91 (d, J=8.9 Hz, 2H), 6.72 (s, 2H), 4.81 (s, 2H), 3.89-3.77 (m, 7H), 3.71-3.62 (m, 4H), 3.59 (s, 3H), 3.52 (s, 2H), 3.41-3.36 (m, 1H), 3.20 (t, J=10.1 Hz, 1H), 3.07 (t, J=6.7 Hz, 1H), 2.76-2.65 (m, 4H), 2.14 (t, J=10.2 Hz, 2H), 1.86-1.70 (m, 4H), 1.46-1.22 (m, 4H).

### Compound A36: 1-(3-(4-((1-(2,6-Dimethoxy-4-(2-methyl-1-oxo-1,2-dihydro-2,7-naphthyridin-4-yl)benzyl)piperidin-4-yl)oxy)piperidine-1-carbonyl)phenyl)dihydropyrimidine-2,4(1H,3H)-dione

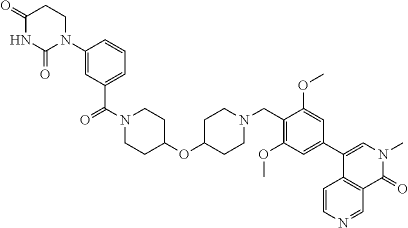 [SMILES: COc1cc(-c2cn(C)c(=O)c3cnccc23)cc(OC)c1CN1CCC(OC2CCN(C(=O)c3cccc(N4CCC(=O)NC4=O)c3)CC2)CC1]

### Step 1: tert-butyl 4-((1-(3-(2,4-dioxotetrahydropyrimidin-1(2H)-yl)benzoyl)piperidin-4-yl)oxy)piperidine-1-carboxylate

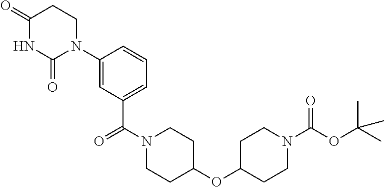 [SMILES: CC(C)(C)OC(=O)N1CCC(OC2CCN(C(=O)c3cccc(N4CCC(=O)NC4=O)c3)CC2)CC1]

To a 100 ml round bottom flask were added 3-(2,4-dioxotetrahydropyrimidin-1(2H)-yl)benzoic acid (intermediate 22, 247 mg, 1.05 mmol), tert-butyl 4-(piperidin-4-yloxy)piperidine-1-carboxylate (intermediate 1, 300 mg, 1.05 mmol), TEA (0.6 ml, 4.2 mmol), HATU (478 mg, 1.26 mmol) and DMF (10 ml). The RM was stirred at RT for 1 h, then EtOAc (60 ml) was added and the organic phase was washed with brine (3×20 ml), dried over Na₂SO₄ and the solid residue (0.5 g), containing the title compound, was directly used for the next step without further purification.

Method I: Rt=1.65 min; [M+Na]⁺=523.

### Step 2: 1-(3-(4-(piperidin-4-yloxy)piperidine-1-carbonyl)phenyl)dihydropyrimidine-2,4(1H,3H)-dione

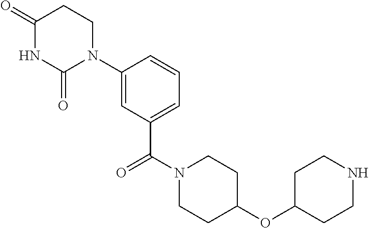 [SMILES: O=C1CCN(c2cccc(C(=O)N3CCC(OC4CCNCC4)CC3)c2)C(=O)N1]

To a 100 ml round bottom flask were added tert-butyl 4-((1-(3-(2,4-dioxotetrahydropyrimidin-1(2H)-yl)benzoyl)piperidin-4-yl)oxy)piperidine-1-carboxylate (500 mg, 0.89 mmol), a solution of HCl (4 M) in 1,4-dioxane (10 ml) and DCM (20 ml) and the RM was stirred at RT for 2 h. The mixture was concentrated to dryness, yielding the title compound as the corresponding hydrochloride salt (550 mg), which was directly used for the next step without further purification.

Method J: Rt=0.76 min; [M+H]⁺=401.

### Step 3: 1-(3-(4-((1-(2,6-dimethoxy-4-(2-methyl-1-oxo-1,2-dihydro-2,7-naphthyridin-4-yl)benzyl)piperidin-4-yl)oxy)piperidine-1-carbonyl)phenyl)dihydropyrimidine-2,4(1H,3H)-dione

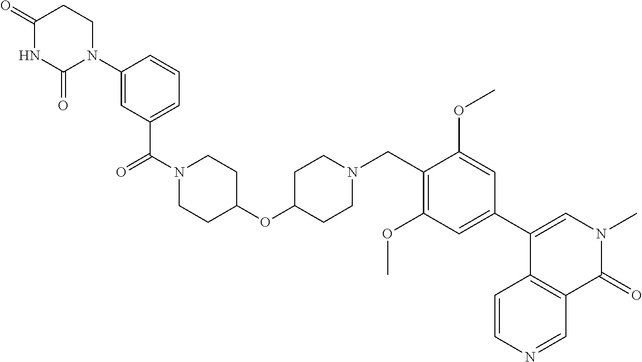 [SMILES: COc1cc(-c2cn(C)c(=O)c3cnccc23)cc(OC)c1CN1CCC(OC2CCN(C(=O)c3cccc(N4CCC(=O)NC4=O)c3)CC2)CC1]

To a 50 ml round bottom flask were added 2,6-dimethoxy-4-(2-methyl-1-oxo-1,2-dihydro-2,7-naphthyridin-4-yl)benzaldehyde (intermediate 11, 150 mg, 0.46 mmol), 1-(3-(4-(piperidin-4-yloxy)piperidine-1-carbonyl)phenyl)dihydropyrimidine-2,4(1H,3H)-dione hydrochloride (200 mg, 0.46 mmol), TEA (0.07 ml, 0.5 mmol), a solution of ZnCl₂ (1 M) in THE (0.567 ml, 0.567 mmol) and DMSO (3 ml). The RM was stirred at room temperature for 3 h, solid NaBH₃CN (145 mg, 2.3 mmol) and MeOH (2 ml) were added and the RM was stirred at RT for 16 h. The mixture was concentrated and the residue was purified by preparative HPLC on an XBridge C18 column (21.2×250 mm, 10 μm) eluting with ACN (from 5 to 95%) in an aq. solution of NH₄HCO₃ (10 mM), yielding the title compound as a solid (60 mg).

Method G: Rt=1.57 min; [M+H]⁺=709.

¹H NMR (500 MHz, DMSO-d₆) δ 10.43 (s, 1H), 9.44 (s, 1H), 8.72 (d, J=6 Hz, 1H), 7.87 (s, 1H), 7.58 (d, J=5.5 Hz, 1H), 7.4-7.35 (m, 3H), 7.23 (d, J=7.5 Hz, 1H), 6.71 (s, 2H), 3.98 (br, 1H), 3.83-3.79 (m, 8H), 3.67 (br, 1H), 3.59 (br, 3H), 3.51 (br, 3H), 3.38 (br, 1H), 3.21-3.15 (m, 2H), 2.72 (m, 4H), 2.13 (m, 2H), 1.84-1.74 (s, 4H), 1.37 (br, 4H).

### Compound A37: 1-(5-(4-((1-(3-(2,6-Dimethoxy-4-(2-methyl-1-oxo-1,2-dihydro-2,7-naphthyridin-4-yl)phenyl)propyl)piperidin-4-yl)oxy)piperidine-1-carbonyl)-2-methoxyphenyl)dihydropyrimidine-2,4(1H,3H)-dione

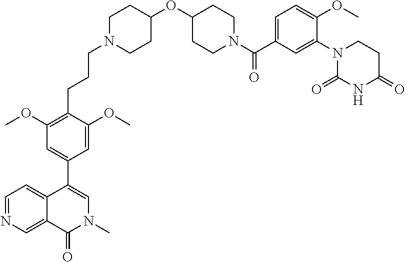 [SMILES: COc1ccc(C(=O)N2CCC(OC3CCN(CCCc4c(OC)cc(-c5cn(C)c(=O)c6cnccc56)cc4OC)CC3)CC2)cc1N1CCC(=O)NC1=O]

### Step 1: methyl (E)-3-(4-bromo-2,6-dimethoxyphenyl)acrylate

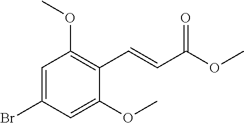 [SMILES: COC(=O)/C=C/c1c(OC)cc(Br)cc1OC]

To a 250 ml round bottom flask were added 4-bromo-2,6-dimethoxybenzaldehyde (3 g, 12.24 mmol), THE (100 ml) and NaH (60% dispersion in mineral oil, 2 g, 48.97 mmol). The RM was stirred at RT for 30 min and cooled to 0° C. A solution of methyl 2-(dimethoxyphosphoryl)acetate in THF (20 ml) was added and the RM was allowed to reach RT and stirring was continued for 16 h. The mixture was again cooled to 0° C. and an aq. sat. solution of NH₄Cl was added. The mixture was extracted with EtOAc (2×50 ml), the combined organic phases were concentrated and the residue was purified by chromatography on silica gel eluting with EtOAc (from 0% to 10%) in PE, yielding the title compound as a solid (3.3 g).

Method G: Rt=2.06 min; [M+H]⁺=301.

### Step 2: methyl (E)-3-(2,6-dimethoxy-4-(4,4,5,5-tetramethyl-1,3,2-dioxaborolan-2-yl)phenyl)acrylate

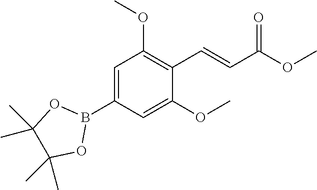

To a 100 ml round bottom flask were added under an argon atmosphere methyl (E)-3-(4-bromo-2,6-dimethoxyphenyl)acrylate (1.5 g, 4.98 mmol), BISPIN (1.52 g, 5.98 mmol), KOAc (1.47 g, 14.94 mmol), PdCl₂(dppf) (37 mg, 0.05 mmol) and 1,4-dioxane (40 ml). The RM was stirred at 90° C. for 16 h. The mixture was filtered, the solids were washed with EtOAc (50 ml) and the combined filtrates were concentrated and the residue was purified by chromatography on silica gel eluting with EtOAc (from 0% to 15%) in PE, yielding the title compound as a solid (1.38 g).

Method G: Rt=2.15 min; [M+H]⁺=349.

### Step 3: methyl 3-(2,6-dimethoxy-4-(4,4,5,5-tetramethyl-1,3,2-dioxaborolan-2-yl)phenyl)propanoate

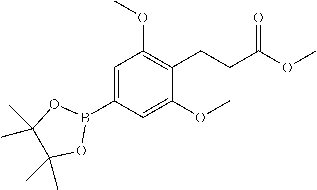

To a 50 ml round bottom flask were added methyl (E)-3-(2,6-dimethoxy-4-(4,4,5,5-tetramethyl-1,3,2-dioxaborolan-2-yl)phenyl)acrylate (1.3 g, 3.74 mmol), Pd/C (10%, 200 mg) and MeOH (30 ml). The RM was stirred under a H₂ atmosphere (1 bar) at 50° C. for 2 h, filtered and the filtrate was concentrated, yielding the title compound as a solid (1.2 g).

Method G: Rt=2.12 min; [M+H]⁺=351.

### Step 4: (4-(3-hydroxypropyl)-3,5-dimethoxyphenyl)boronic Acid

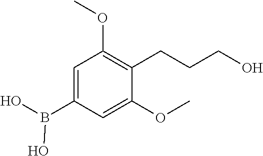 [SMILES: COc1cc(B(O)O)cc(OC)c1CCCO]

To a 50 ml round bottom flask were added methyl 3-(2,6-dimethoxy-4-(4,4,5,5-tetramethyl-1,3,2-dioxaborolan-2-yl)phenyl)propanoate (1.2 g, 3.43 mmol) and THF (30 ml). The RM was cooled to 0° C. and LiAlH₄ (390 mg, 10.28 mmol) was added portionswise. The RM was stirred at RT for 6 h, cooled to 0° C. and water was carefully added. The RM was extracted with EtOAc (2×50 ml) and the combined organic phases were concentrated, yielding the title compound as a solid (750 mg).

Method G: Rt=1.97 min; [M+H]⁺=241.

### Step 5: 4-(4-(3-hydroxypropyl)-3,5-dimethoxyphenyl)-2-methyl-2,7-naphthyridin-1(2H)-one

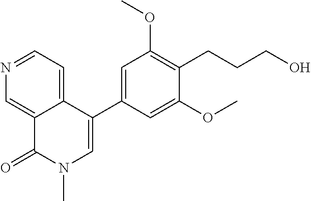 [SMILES: COc1cc(-c2cn(C)c(=O)c3cnccc23)cc(OC)c1CCCO]

To a 50 ml round bottom flask were added under an argon atmosphere (4-(3-hydroxypropyl)-3,5-dimethoxyphenyl)boronic acid (650 mg, 2.71 mmol), 4-bromo-2-methyl-2,7-naphthyridin-1(2H)-one (intermediate 5, 647 mg, 2.71 mmol), Na₂CO₃ (720 mg, 6.77 mmol), PdCl₂(dppf) (99 mg, 0.14 mmol), 1,4-dioxane (15 ml) and water (3 ml). The RM was stirred at 80° C. for 16 h, filtered, the solids were washed with EtOAc (200 ml), the combined organic phases were dried over MgSO₄ and concentrated, yielding the title compound as a solid (650 mg).

Method G: Rt=1.64 min; [M+H]⁺=355.

### Step 6: 3-(2,6-dimethoxy-4-(2-methyl-1-oxo-1,2-dihydro-2,7-naphthyridin-4-yl)phenyl)propanal

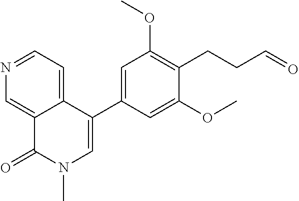 [SMILES: COc1cc(-c2cn(C)c(=O)c3cnccc23)cc(OC)c1CCC=O]

To a 25 ml round bottom flask were added 4-(4-(3-hydroxypropyl)-3,5-dimethoxyphenyl)-2-methyl-2,7-naphthyridin-1(2H)-one (300 mg, 0.85 mmol), IBX (476 mg, 1.7 mmol) and DMSO (5 ml). The RM was stirred at 50° C. for 4 h. An aq. sat. solution of NaCl (80 ml) was added and the aq. phase was extracted with EtOAc (3×50 ml), the combined organic phases were concentrated, yielding the title compound as a solid (270 mg), which was directly used for the next step without further purification.

Method G: Rt=1.73 min; [M+H]⁺=353.

### Step 7: 1-(5-(4-((1-(3-(2,6-dimethoxy-4-(2-methyl-1-oxo-1,2-dihydro-2,7-naphthyridin-4-yl)phenyl)propyl)piperidin-4-yl)oxy)piperidine-1-carbonyl)-2-methoxyphenyl)dihydropyrimidine-2,4(1H,3H)-dione

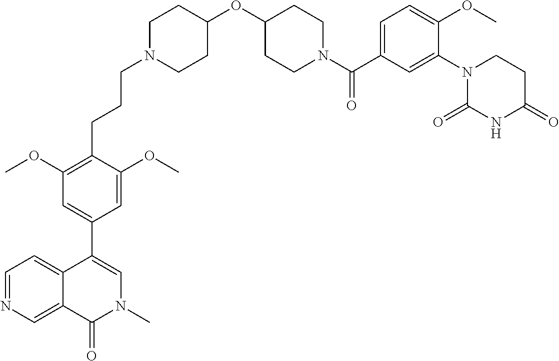 [SMILES: COc1ccc(C(=O)N2CCC(OC3CCN(CCCc4c(OC)cc(-c5cn(C)c(=O)c6cnccc56)cc4OC)CC3)CC2)cc1N1CCC(=O)NC1=O]

To a 25 ml round bottom flask were added 1-(2-methoxy-5-(4-(piperidin-4-yloxy)piperidine-1-carbonyl)phenyl)dihydropyrimidine-2,4(1H,3H)-dione hydrochloride (intermediate 29,240 mg, 0.51 mmol), K₂CO₃ (85 mg, 0.62 mmol) and DMSO (4 ml). The RM was stirred at RT for 10 min, 3-(2,6-dimethoxy-4-(2-methyl-1-oxo-1,2-dihydro-2,7-naphthyridin-4-yl)phenyl)propanal (270 mg, 0.77 mmol) and a solution of ZnCl₂ (1.3 ml) in THE (1 M) were added and the RM was stirred at RT for 30 min. Solid NaBH₃CN (263 mg, 4.11 mmol) was added and the RM was stirred at RT for 16 h. The mixture was filtered, the solids were washed with EtOAc, the combined filtrates were concentrated and the residue was purified by preparative HPLC on an Xtimate C18 column (250×21.2 mm, 10 μm) eluting with ACN (from 5% to 80%) in an aq. solution of NH₄HCO₃ (10 mM), yielding the title compound as a solid (70 mg).

Method G: Rt=1.70 min; [M+H]⁺=767.

¹H NMR (500 MHz, DMSO-d₆) δ 10.33 (s, 1H), 9.44 (s, 1H), 8.71 (d, J=5.7 Hz, 1H), 7.84 (s, 1H), 7.56 (d, J=5.6 Hz, 1H), 7.41-7.31 (m, 2H), 7.15 (d, J=8.6 Hz, 1H), 6.69 (s, 2H), 3.84 (s, 3H), 3.80 (s, 6H), 3.73-3.63 (m, 2H), 3.62-3.57 (m, 5H), 3.42 (s, 2H), 3.20 (t, J=9.8 Hz, 2H), 2.74-2.64 (m, 4H), 2.62-2.56 (m, 2H), 2.28 (t, J=7.2 Hz, 2H), 2.05-1.94 (m, 2H), 1.78 (s, 4H), 1.63-1.53 (m, 2H), 1.41 (d, J=9.5 Hz, 4H).

### Compound A38: 1-(2-Chloro-5-(4-((1-(2,6-dimethoxy-4-(2-methyl-1-oxo-1,2-dihydro-2,7-naphthyridin-4-yl)phenethyl)piperidin-4-yl)oxy)piperidine-1-carbonyl)phenyl)dihydropyrimidine-2,4(1H,3H)-dione

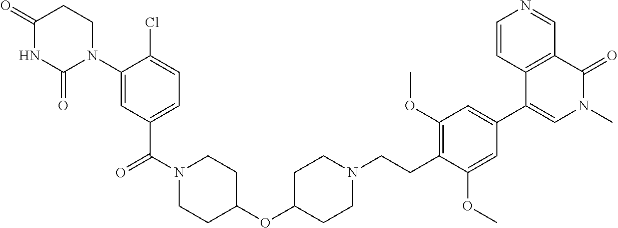 [SMILES: COc1cc(-c2cn(C)c(=O)c3cnccc23)cc(OC)c1CCN1CCC(OC2CCN(C(=O)c3ccc(Cl)c(N4CCC(=O)NC4=O)c3)CC2)CC1]

To a 50 ml round bottom flask were added 2-(2,6-dimethoxy-4-(2-methyl-1-oxo-1,2-dihydro-2,7-naphthyridin-4-yl)phenyl)acetaldehyde (intermediate 14, 100 mg, 0.29 mmol), 1-(2-chloro-5-(4-(piperidin-4-yloxy)piperidine-1-carbonyl)phenyl)dihydropyrimidine-2,4(1H,3H)-dione hydrochloride (intermediate 28, 120 mg, 0.27 mmol), a solution of ZnCl₂ (1 M) in THE (0.38 ml, 0.38 mmol) and DMSO (2 ml). The RM was stirred at RT for 30 min, solid NaBH₃CN (73 mg, 1.16 mmol) and MeOH (1 ml) were added and the RM was stirred at RT for 16 h, concentrated and the residue was purified by preparative HPLC on an Xtimate C18 column (250×21.2 mm, 10 μm) eluting with ACN (from 5% to 80%) in an aq. solution of NH₄HCO₃ (10 mM), yielding the title compound as a solid (30 mg).

Method G: Rt=1.67 min; [M+H]⁺=757.

¹H NMR (500 MHz, DMSO-d₆) δ 10.51 (s, 1H), 9.44 (s, 1H), 8.71 (d, J=5.7 Hz, 1H), 7.84 (s, 1H), 7.64 (d, J=8.2 Hz, 1H), 7.57 (s, 1H), 7.54 (d, J=5.7 Hz, 1H), 7.40 (dd, J=8.2, 2.0 Hz 1H), 6.69 (s, 2H), 4.10-3.90 (m, 1H), 3.80 (s, 8H), 3.77-3.67 (m, 4H), 3.55-3.40 (m, 2H), 3.28-3.10 (m, 3H), 2.83-2.69 (m, 5H), 2.38-2.29 (m, 2H), 2.17-1.99 (m, 2H), 1.90-1.72 (m, 4H), 1.51-1.36 (m, 4H).

### Compound A39: 1-(2-Chloro-5-(4-((1-(2,5-dimethoxy-4-(2-methyl-1-oxo-1,2-dihydro-2,7-naphthyridin-4-yl)phenethyl)piperidin-4-yl)oxy)piperidine-1-carbonyl)phenyl)dihydropyrimidine-2,4(1H,3H)-dione

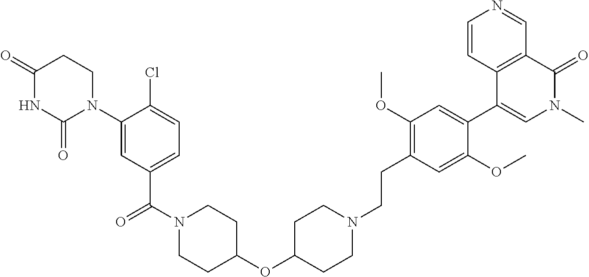 [SMILES: COc1cc(-c2cn(C)c(=O)c3cnccc23)c(OC)cc1CCN1CCC(OC2CCN(C(=O)c3ccc(Cl)c(N4CCC(=O)NC4=O)c3)CC2)CC1]

To a 100 ml round bottom flask were added 1-(2-chloro-5-(4-(piperidin-4-yloxy)piperidine-1-carbonyl)phenyl)dihydropyrimidine-2,4(1H,3H)-dione (intermediate 28, 200 mg, 0.42 mmol), TEA (0.065 ml, 0.47 mmol) and DMSO (5 ml). The RM was stirred at RT for 10 min, 2-(2,5-dimethoxy-4-(2-methyl-1-oxo-1,2-dihydro-2,7-naphthyridin-4-yl)phenyl)acetaldehyde (intermediate 15, 144 mg, 0.42 mmol) and a solution of ZnCl₂ (1 M) in THF (0.636 ml, 0.636 mmol) was added and the RM was stirred at RT for 2 h. Solid NaBH₃CN (135 mg, 2.14 mmol) was added and the RM was stirred at RT for 16 h. The mixture was directly purified by preparative HPLC on an Xtimate C18 column (250×21.2 mm, 10 μm) eluting with ACN (from 5% to 80%) in an aq. solution of NH₄HCO₃ (10 mM), yielding the title compound as a solid (34 mg).

Method G: Rt=1.64 min; [M+H]⁺=757.

¹H NMR (400 MHz, DMSO-d₆) δ 10.24 (s, 1H), 9.39 (s, 1H), 8.63 (d, J=4.4 Hz, 1H), 7.71 (s, 1H), 7.64 (d, J=6.4 Hz, 1H), 7.57 (br, 1H), 7.41 (d, J=6.4 Hz, 1H), 7.01 (m, 2H), 6.87 (s, 1H), 3.97 (br, 1H), 3.74-3.66 (m, 6H), 3.64-3.56 (m, 8H), 3.47-3.45 (br, 2H), 3.27-3.15 (br, 2H), 2.78-2.72 (br, 6H), 2.14 (br, 2H), 1.83-1.73 (br, 4H), 1.45-1.43 (br, 4H).

### Compound A40: 1-(3-(4-((1-(2,6-Dimethoxy-4-(2-methyl-1-oxo-1,2-dihydro-2,7-naphthyridin-4-yl)phenethyl)piperidin-4-yl)oxy)piperidine-1-carbonyl)phenyl)dihydropyrimidine-2,4(1H,3H)-dione

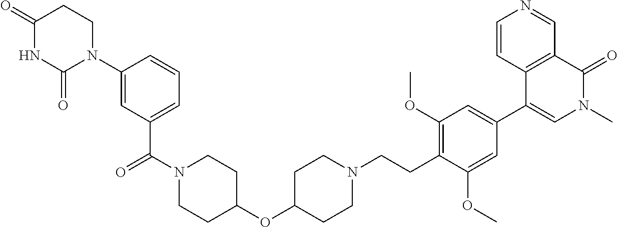 [SMILES: COc1cc(-c2cn(C)c(=O)c3cnccc23)cc(OC)c1CCN1CCC(OC2CCN(C(=O)c3cccc(N4CCC(=O)NC4=O)c3)CC2)CC1]

### Step 1: tert-butyl 4-((1-(2,6-dimethoxy-4-(2-methyl-1-oxo-1,2-dihydro-2,7-naphthyridin-4-yl)phenethyl)piperidin-4-yl)oxy)piperidine-1-carboxylate

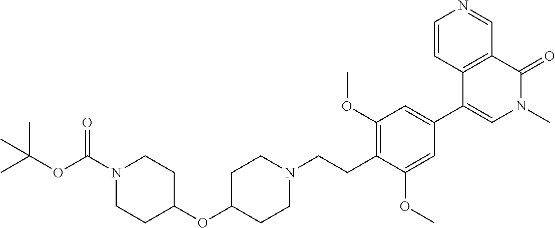 [SMILES: COc1cc(-c2cn(C)c(=O)c3cnccc23)cc(OC)c1CCN1CCC(OC2CCN(C(=O)OC(C)(C)C)CC2)CC1]

To a 250 ml round bottom flask were added 2-(2,6-dimethoxy-4-(2-methyl-1-oxo-1,2-dihydro-2,7-naphthyridin-4-yl)phenyl)acetaldehyde (intermediate 14, 170 mg, 0.5 mmol), tert-butyl 4-(piperidin-4-yloxy)piperidine-1-carboxylate (intermediate 1, 143 mg, 0.5 mmol), K₂CO₃ (138 mg, 1 mmol), a solution of ZnCl₂ (1 M) in THE (0.65 ml, 0.65 mmol) and DMSO (3 ml). The RM was stirred at RT for 30 min, solid NaBH₃CN (126 mg, 2 mmol) was added and the RM was stirred at RT for 16 h. The mixture was concentrated and the residue was purified by preparative HPLC on an XBridge C18 column (250×21.2 mm, 10 μm) eluting with ACN (from 5% to 95%) in an aq. solution of NH₄HCO₃ (10 mM), yielding the title compound as a solid (100 mg).

Method G: Rt=2.10 min; [M+H]⁺=607.

### Step 2: 4-(3,5-dimethoxy-4-(2-(4-(piperidin-4-yloxy)piperidin-1-yl)ethyl)phenyl)-2-methyl-2,7-naphthyridin-1(2H)-one

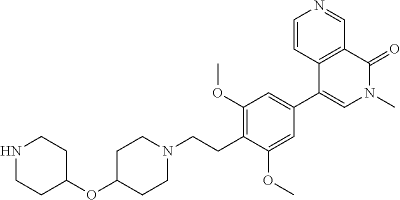 [SMILES: COc1cc(-c2cn(C)c(=O)c3cnccc23)cc(OC)c1CCN1CCC(OC2CCNCC2)CC1]

To a 50 ml round bottom flask were added tert-butyl 4-((1-(2,6-dimethoxy-4-(2-methyl-1-oxo-1,2-dihydro-2,7-naphthyridin-4-yl)phenethyl)piperidin-4-yl)oxy)piperidine-1-carboxylate (100 mg, 0.165 mmol), DCM (8 ml) and a solution of HCl (4 M) in 1,4-dioxane (4 ml). The RM was stirred at RT for 1 h and evaporated to dryness, yielding the corresponding hydrochloride salt of the title compound as a solid (100 mg), which was directly used for the next step without further purification.

Method E: Rt=1.95 min; [M+H]⁺=507.

### Step 3:1-(3-(4-((1-(2,6-dimethoxy-4-(2-methyl-1-oxo-1,2-dihydro-2,7-naphthyridin-4-yl)phenethyl)piperidin-4-yl)oxy)piperidine-1-carbonyl)phenyl)dihydropyrimidine-2,4(1H,3H)-dione

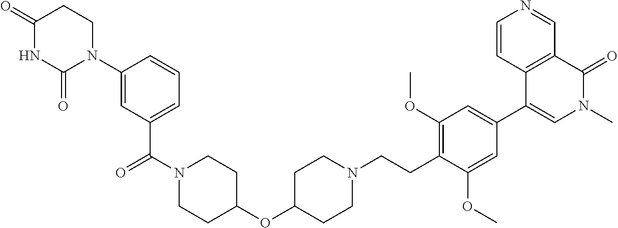 [SMILES: COc1cc(-c2cn(C)c(=O)c3cnccc23)cc(OC)c1CCN1CCC(OC2CCN(C(=O)c3cccc(N4CCC(=O)NC4=O)c3)CC2)CC1]

To a 50 ml round bottom flask were added 4-(3,5-dimethoxy-4-(2-(4-(piperidin-4-yloxy)piperidin-1-yl)ethyl)phenyl)-2-methyl-2,7-naphthyridin-1(2H)-one hydrochloride (100 mg, 0.16 mmol), 3-(2,4-dioxotetrahydropyrimidin-1(2H)-yl)benzoic acid (intermediate 22, 37 mg, 0.16 mmol), DIEA (103 mg, 0.8 mmol) and DMF (2 ml). The RM was stirred at RT for 5 min, solid HATU (72 mg, 0.19 mmol) was added and the RM was stirred at RT for 2 h. The mixture was concentrated and the residue was purified by preparative HPLC on an XBridge C18 column (250×21.2 mm, 10 μm) eluting with ACN (from 5% to 80%) in an aq. solution of NH₄HCO₃ (10 mM), yielding the title compound as a solid (38 mg).

Method G: Rt=1.62 min; [M+H]⁺=723.

¹H NMR (500 MHz, DMSO-d₆) δ 10.42 (s, 1H), 9.44 (s, 1H), 8.71 (d, J=5.7 Hz, 1H), 7.84 (s, 1H), 7.55 (d, J=5.7 Hz, 1H), 7.48-7.34 (m, 3H), 7.23 (d, J=7.4 Hz, 1H), 6.69 (s, 1H), 4.08-3.90 (m, 1H), 3.87-3.76 (m, 8H), 3.74-3.64 (m, 1H), 3.59 (s, 3H), 3.55-3.38 (m, 3H), 3.27-3.10 (m, 2H), 2.86-2.68 (m, 6H), 2.40-2.28 (m, 2H), 2.11 (t, J=10.3 Hz, 2H), 1.90-1.69 (m, 4H), 1.50-1.33 (m, 4H).

### Compound A41: 1-(5-(4-((1-(2,5-Dimethoxy-4-(2-methyl-1-oxo-1,2-dihydro-2,7-naphthyridin-4-yl)phenethyl)piperidin-4-yl)oxy)piperidine-1-carbonyl)-2-methoxyphenyl)dihydropyrimidine-2,4(1H,3H)-dione

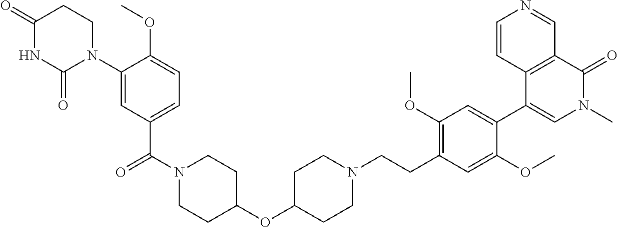 [SMILES: COc1cc(-c2cn(C)c(=O)c3cnccc23)c(OC)cc1CCN1CCC(OC2CCN(C(=O)c3ccc(OC)c(N4CCC(=O)NC4=O)c3)CC2)CC1]

To a 100 ml round bottom flask were added 1-(2-methoxy-5-(4-(piperidin-4-yloxy)piperidine-1-carbonyl)phenyl)dihydropyrimidine-2,4(1H,3H)-dione hydrochloride (intermediate 29, 100 mg, 0.17 mmol), DMSO (4 ml), MeOH (1 ml), 2-(2,5-dimethoxy-4-(2-methyl-1-oxo-1,2-dihydro-2,7-naphthyridin-4-yl)phenyl)acetaldehyde (intermediate 15, 56 mg, 0.17 mmol) and a solution of ZnCl₂ (1 M) in TH (0.26 ml, 0.26 mmol). The RM was stirred at RT for 2 h, solid NaBH₃CN (55 mg, 0.88 mmol) was added and the RM was stirred at RT for 16 h. The mixture was directly purified by preparative HPLC on an Xtimate C18 column (250×21.2 mm, 10 μm) eluting with ACN (from 5% to 80%) in an aq. solution of NH₄HCO₃ (10 mM), yielding the title compound as a solid (15 mg).

Method A: Rt=1.13 min; [M+H]⁺=753.

¹H NMR (400 MHz, DMSO-d₆) δ 10.33 (s, 1H), 9.39 (s, 1H), 8.63 (d, J=4.4 Hz, 1H), 7.71 (s, 1H), 7.39 (d, J=1.6 Hz, 1H), 7.34 (br, 1H), 7.16 (d, J=6.4 Hz, 1H), 7.02-7.01 (m, 2H), 6.87 (s, 1H), 3.84 (br, 4H), 3.74-3.68 (m, 5H), 3.62-3.56 (m, 10H), 3.46-3.44 (br, 1H), 3.3 (br, 1H), 3.23-3.19 (m, 2H), 2.80-2.76 (br, 4H), 2.15-2.11 (m, 2H), 1.83-1.81 (br, 4H), 1.45-1.41 (br, 4H).

### Compound A42: 1-(5-(4-((1-(2,6-Dimethoxy-4-(2-methyl-1-oxo-1,2-dihydro-2,7-naphthyridin-4-yl)benzyl)piperidin-4-yl)oxy)piperidine-1-carbonyl)-2-methoxyphenyl)dihydropyrimidine-2,4(1H,3H)-dione

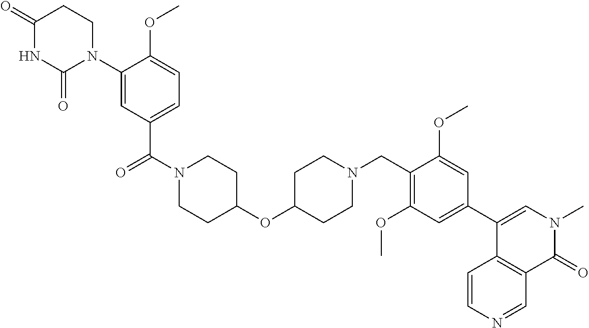 [SMILES: COc1ccc(C(=O)N2CCC(OC3CCN(Cc4c(OC)cc(-c5cn(C)c(=O)c6cnccc56)cc4OC)CC3)CC2)cc1N1CCC(=O)NC1=O]

To a 50 ml round bottom flask were added 2,6-dimethoxy-4-(2-methyl-1-oxo-1,2-dihydro-2,7-naphthyridin-4-yl)benzaldehyde (intermediate 11, 150 mg, 0.46 mmol), 1-(2-methoxy-5-(4-(piperidin-4-yloxy)piperidine-1-carbonyl)phenyl)dihydropyrimidine-2,4(1H,3H)-dione hydrochloride (intermediate 29, 237 mg, 0.51 mmol), K₂CO₃ (191 mg, 1.38 mmol), a solution of ZnCl₂ (1 M) in THE (0.51 ml, 0.51 mmol) and DMSO (3 ml). The RM was stirred at RT for 3 h, solid NaBH₃CN (58 mg, 0.92 mmol) and HOAc (28 mg, 0.46 mmol) were added and the RM was stirred at 50° C. for 16 h. The mixture was concentrated and the residue was purified by preparative HPLC on an XBridge C18 column (21.2×250 mm, 10 μm) eluting with ACN (from 5% to 95%) in an aq. solution of NH₄HCO₃ (10 mM), yielding the title compound as a solid (80 mg).

Method A: Rt=1.16 min; [M+H]⁺=739.

¹H NMR (500 MHz, DMSO-d₆) δ 10.34 (s, 1H), 9.44 (s, 1H), 8.72 (d, J=5.7 Hz, 1H), 7.88 (s, 1H), 7.57 (d, J=5.7 Hz, 1H), 7.38 (dd, J=8.5, 2.1 Hz, 1H), 7.34 (d, J=2.1 Hz, 1H), 7.15 (d, J=8.6 Hz, 1H), 6.72 (s, 2H), 3.82 (d, J=8.6 Hz, 10H), 3.71-3.49 (m, 9H), 3.40 (s, 1H), 3.19 (t, J=9.9 Hz, 2H), 2.84-2.62 (m, 4H), 2.15 (s, 2H), 1.77 (s, 4H), 1.39 (s, 4H).

### Compound B1: 1-(2-Chloro-5-(4-((1-(2,6-dimethoxy-4-(5-methyl-6-oxo-1-propyl-1,6-dihydropyridin-3-yl)phenethyl)piperidin-4-yl)oxy)piperidine-1-carbonyl)phenyl)dihydropyrimidine-2,4(1H,3H)-dione

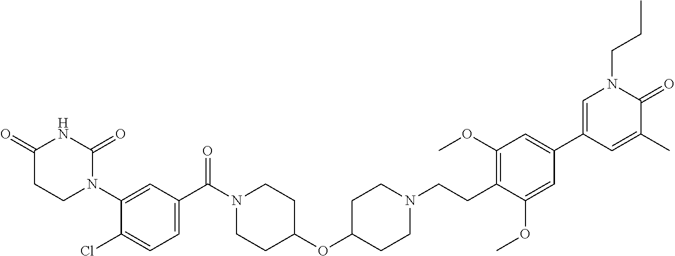 [SMILES: CCCn1cc(-c2cc(OC)c(CCN3CCC(OC4CCN(C(=O)c5ccc(Cl)c(N6CCC(=O)NC6=O)c5)CC4)CC3)c(OC)c2)cc(C)c1=O]

### Step 1:5-bromo-3-methyl-1-propylpyridin-2(1H)-one

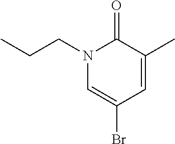

To a 50 ml round bottom flask were added 5-bromo-3-methylpyridin-2(1H)-one (500 mg, 2.67 mmol) and DMF (15 ml) and the mixture was cooled to 0° C. Solid NaH (60% dispersion in mineral oil, 160 mg, 6.67 mmol) was added in small portions and stirring was continued at 0° C. for 1 h. Iodopropane (591 mg, 3.48 mmol) was added dropwise and the RM was stirred at RT for 3 h, added into EtOAc (250 ml), the organic phase was washed with water (2×250 ml) and brine (250 mL), dried over Na₂SO₄ and the residue was purified by chromatography on silica gel eluting with EtOAc (from 0% to 100%) in PE, yielding the title compound as a solid (450 mg).

Method H: Rt=1.75 min; [M+H]⁺=230, 232.

### Step 2:2,6-dimethoxy-4-(5-methyl-6-oxo-1-propyl-1,6-dihydropyridin-3-yl)benzaldehyde

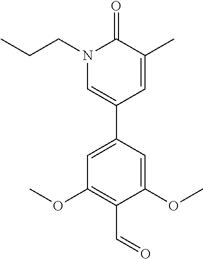 [SMILES: CCCn1cc(-c2cc(OC)c(C=O)c(OC)c2)cc(C)c1=O]

To a 100 ml round bottom flask were added 5-bromo-3-methyl-1-propylpyridin-2(1H)-one (450 mg, 2.4 mmol), 2,6-dimethoxy-4-(4,4,5,5-tetramethyl-1,3,2-dioxaborolan-2-yl)benzaldehyde (intermediate 7, 1.06 g, 3.61 mmol), K₂CO₃ (828 mg, 6 mmol), 1,4-dioxane (10 ml) and PdCl₂(dppf) (175 mg, 0.24 mmol). The RM was stirred under N₂ atmosphere at 100° C. for 16 h, added into water (50 ml) and extracted with EtOAc (150 ml). The organic phase was washed with brine (50 mL), dried over Na₂SO₄ and the residue was purified by chromatography on silica gel eluting with EtOAc (from 0% to 100%) in PE, yielding the title compound as a solid (600 mg).

Method H: Rt=1.69 min; [M+H]⁺=316.

### Step 3: 5-(3,5-dimethoxy-4-(2-methoxyvinyl)phenyl)-3-methyl-1-propylpyridin-2(1H)-one

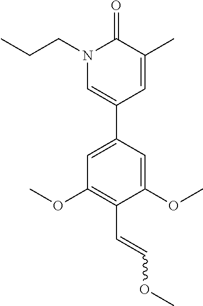 [SMILES: CCCn1cc(-c2cc(OC)c(C=COC)c(OC)c2)cc(C)c1=O]

To a 50 ml round bottom flask were added (methoxymethyl)triphenylphosphonium chloride (1.95 g, 5.7 mmol), t-BuOK (853 mg, 7.6 mmol) and THE (15 ml). The RM was stirred at 0° C. for 30 min and 2,6-dimethoxy-4-(5-methyl-6-oxo-1-propyl-1,6-dihydropyridin-3-yl)benzaldehyde (600 mg, 1.9 mmol) was added. The RM was stirred at 0° C. for 1 h, then at 70° C. for 16 h. The mixture was added into water (100 ml), extracted with EtOAc (2×100 ml), the combined organic phases were washed with brine (100 mL), dried over Na₂SO₄ and the residue was purified by chromatography on silica gel eluting with EtOAc (from 0% to 100%) in PE, yielding the title compound as a solid (1.3 g).

Method H: Rt=1.93 min; [M+H]⁺=344.

### Step 4: 2-(2,6-dimethoxy-4-(5-methyl-6-oxo-1-propyl-1,6-dihydropyridin-3-yl)phenyl)acetaldehyde

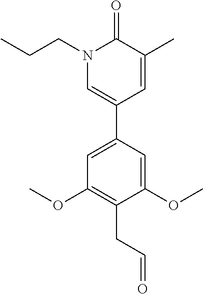 [SMILES: CCCn1cc(-c2cc(OC)c(CC=O)c(OC)c2)cc(C)c1=O]

To a 50 ml round bottom flask were added 5-(3,5-dimethoxy-4-(2-methoxyvinyl)phenyl)-3-methyl-1-propylpyridin-2(1H)-one (1.3 g, 3.79 mmol), acetone (10 ml) and an aq. solution of H₂SO₄ (2 M, 6 ml). The RM was stirred at 65° C. for 3 h, added into water (50 ml), extracted with EtOAc (150 ml), the organic phase was washed with brine (50 mL), dried over Na₂SO₄ and the residue was purified by chromatography on silica gel eluting with EtOAc (from 0% to 100%) in PE, yielding the title compound as a solid (900 mg).

Method A: Rt=1.63 min; [M+H]⁺=330.

### Step 5: tert-butyl 4-((1-(2,6-dimethoxy-4-(5-methyl-6-oxo-1-propyl-1,6-dihydropyridin-3-yl)phenethyl)piperidin-4-yl)oxy)piperidine-1-carboxylate

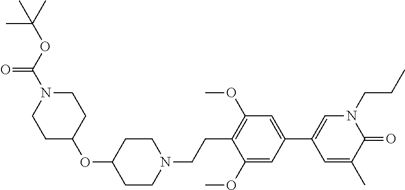 [SMILES: CCCn1cc(-c2cc(OC)c(CCN3CCC(OC4CCN(C(=O)OC(C)(C)C)CC4)CC3)c(OC)c2)cc(C)c1=O]

To a 25 ml round bottom flask were added 2-(2,6-dimethoxy-4-(5-methyl-6-oxo-1-propyl-1,6-dihydropyridin-3-yl)phenyl)acetaldehyde (250 mg, 0.76 mmol), tert-butyl 4-(piperidin-4-yloxy)piperidine-1-carboxylate (intermediate 1, 260 mg, 0.92 mmol), a solution of ZnCl₂ (1 M) in THE (0.99 ml, 0.99 mmol) and DMSO (5 ml). The RM was stirred at RT for 1 h, solid NaBH₃CN (96 mg, 1.52 mmol) was added and the RM was stirred at RT for 16 h. The mixture was added into water (50 ml), extracted with EtOAc (150 ml), the organic phase was washed with brine (50 mL), dried over Na₂SO₄ and the residue was purified by reversed phase chromatography on a Biotage Agela C18 column (120 g, spherical 20-35 μm, 100 Å) eluting with ACN (from 5% to 95%) in aq. TFA (0.1%), yielding the title compound as a solid (170 mg).

Method A: Rt=1.35 min; [M+H]⁺=597.

### Step 6: 5-(3,5-dimethoxy-4-(2-(4-(piperidin-4-yloxy)piperidin-1-yl)ethyl)phenyl)-3-methyl-1-propylpyridin-2(1H)-one

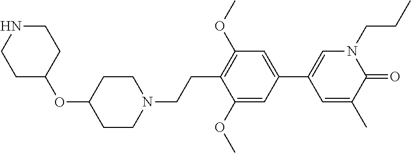 [SMILES: CCCn1cc(-c2cc(OC)c(CCN3CCC(OC4CCNCC4)CC3)c(OC)c2)cc(C)c1=O]

To a 25 mL round bottom flask were added tert-butyl 4-((1-(2,6-dimethoxy-4-(5-methyl-6-oxo-1-propyl-1,6-dihydropyridin-3-yl)phenethyl)piperidin-4-yl)oxy)piperidine-1-carboxylate (50 mg, 0.08 mmol), a solution of HCl (4 M) in 1,4-dioxane (1 ml), DCM (2 ml) and MeOH (0.5 ml). The RM was stirred at RT for 5 h and evaporated to dryness, yielding the corresponding hydrochloride salt of the title compound as a solid (50 mg), which was used for the next step without further purification.

Method A: Rt=1.11 min; [M+H]⁺=497.

### Step 7: 1-(2-chloro-5-(4-((1-(2,6-dimethoxy-4-(5-methyl-6-oxo-1-propyl-1,6-dihydropyridin-3-yl)phenethyl)piperidin-4-yl)oxy)piperidine-1-carbonyl)phenyl)dihydropyrimidine-2,4(1H,3H)-dione

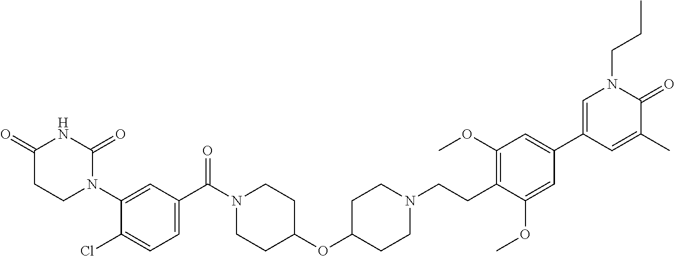 [SMILES: CCCn1cc(-c2cc(OC)c(CCN3CCC(OC4CCN(C(=O)c5ccc(Cl)c(N6CCC(=O)NC6=O)c5)CC4)CC3)c(OC)c2)cc(C)c1=O]

To a 25 ml round bottom flask were added 5-(3,5-dimethoxy-4-(2-(4-(piperidin-4-yloxy)piperidin-1-yl)ethyl)phenyl)-3-methyl-1-propylpyridin-2(1H)-one hydrochloride (50 mg, 0.12 mmol), perfluorophenyl 4-chloro-3-(2,4-dioxotetrahydropyrimidin-1(2H)-yl)benzoate (intermediate 38, 52 mg, 0.12 mmol), DIEA (52 mg, 0.40 mmol) and DMF (3 ml). The RM was stirred at RT for 6 h, the solvent was removed and the residue was purified by preparative HPLC on an XBridge C18 column (250×21.2 mm, 10 μm) eluting with ACN (from 5% to 95%) in aq. NH₄HCO₃ (0.01 M), yielding the title compound as a solid (30 mg).

Method H: Rt=1.90 min; [M+H]⁺=748.

¹H NMR (500 MHz, DMSO) δ 10.52 (s, 1H), 7.96 (d, J=2.3 Hz, 1H), 7.77 (s, 1H), 7.64 (d, J=8.2 Hz, 1H), 7.57 (s, 1H), 7.40 (dd, J=8.2, 2.0 Hz, 1H), 6.77 (s, 2H), 3.94-3.90 (m, 2H), 3.84 (s, 6H), 3.67 (dd, J=49.9, 16.1 Hz, 4H), 3.46 (s, 2H), 3.27-3.11 (m, 2H), 2.74 (dd, J=14.1, 7.0 Hz, 6H), 2.36 (s, 4H), 2.09 (s, 3H), 1.85-1.68 (m, 6H), 1.43 (s, 4H), 0.89 (t, J=7.4 Hz, 3H).

### Compound B2: 1-(2-Chloro-5-(4-(1-(4-(4,5-dimethyl-6-oxo-1-propyl-1,6-dihydropyridin-3-yl)-2,6-dimethoxyphenethyl)piperidin-4-yloxy)piperidine-1-carbonyl)phenyl)dihydropyrimidine-2,4(1H,3H)-dione

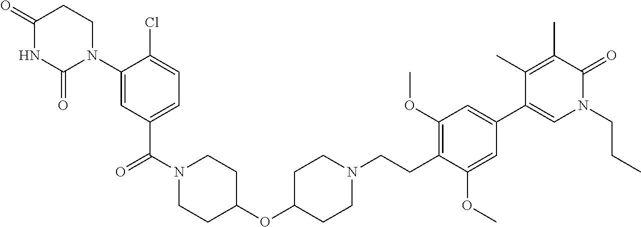 [SMILES: CCCn1cc(-c2cc(OC)c(CCN3CCC(OC4CCN(C(=O)c5ccc(Cl)c(N6CCC(=O)NC6=O)c5)CC4)CC3)c(OC)c2)c(C)c(C)c1=O]

### Step 1: tert-butyl 4-(1-(4-(4,5-dimethyl-6-oxo-1-propyl-1,6-dihydropyridin-3-yl)-2,6-dimethoxyphenethyl)piperidin-4-yloxy)piperidine-1-carboxylate

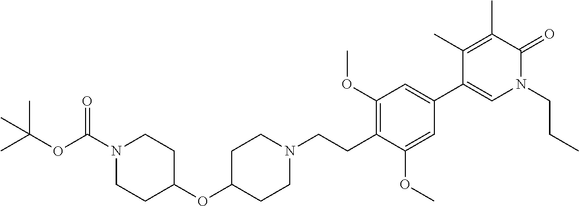 [SMILES: CCCn1cc(-c2cc(OC)c(CCN3CCC(OC4CCN(C(=O)OC(C)(C)C)CC4)CC3)c(OC)c2)c(C)c(C)c1=O]

To a 50 ml round bottom flask were added under a argon atmosphere 5-bromo-3,4-dimethyl-1-propylpyridin-2(1H)-one (intermediate 31, 204 mg, 0.36 mmol), tert-butyl 4-((1-(2,6-dimethoxy-4-(4,4,5,5-tetramethyl-1,3,2-dioxaborolan-2-yl)phenethyl)piperidin-4-yl)oxy)piperidine-1-carboxylate (intermediate 34, 67 mg, 0.27 mmol), PdCl₂(dppf)(6 mg, 0.0068 mmol), K₂CO₃ (75 mg, 0.55 mmol), 1,4-dioxane (16 ml) and water (4 ml). The RM was stirred at 100° C. for 18 h. The mixture was cooled to RT, concentrated and added into water (60 ml). The mixture was extracted with EtOAc (3×20 ml), the combined organic phases were concentrated and the residue was purified by silica gel chromatography eluting with EtOAc (from 0% to 80%) in PE, yielding the title compound as an oil (62 mg).

Method E: Rt=1.64 min; [M+H]⁺=612.

### Step 2:5-(3,5-dimethoxy-4-(2-(4-(piperidin-4-yloxy)piperidin-1-yl)ethyl)phenyl)-3,4-dimethyl-1-propylpyridin-2(1H)-one

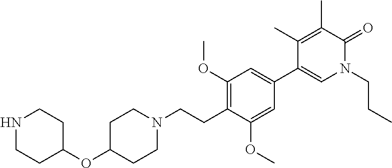 [SMILES: CCCn1cc(-c2cc(OC)c(CCN3CCC(OC4CCNCC4)CC3)c(OC)c2)c(C)c(C)c1=O]

To a 50 ml round bottom flask were added tert-butyl 4-(1-(4-(4,5-dimethyl-6-oxo-1-propyl-1,6-dihydropyridin-3-yl)-2,6-dimethoxyphenethyl)piperidin-4-yloxy)piperidine-1-carboxylate (62 mg, 0.10 mmol), DCM (2 ml), MeOH (0.5 ml) and a solution of HCl (4 M) in 1,4-dioxane (2 ml). The RM was stirred at RT for 6 h and concentrated to dryness, yielding the corresponding hydrochloride salt of the title compound as a solid (60 mg), which was used for the next step without further purification.

Method E: Rt=1.43 min; [M+H]⁺=512.

### Step 3:1-(2-chloro-5-(4-(1-(4-(4,5-dimethyl-6-oxo-1-propyl-1,6-dihydropyridin-3-yl)-2,6-dimethoxyphenethyl)piperidin-4-yloxy)piperidine-1-carbonyl)phenyl)-dihydropyrimidine-2,4(1H,3H)-dione

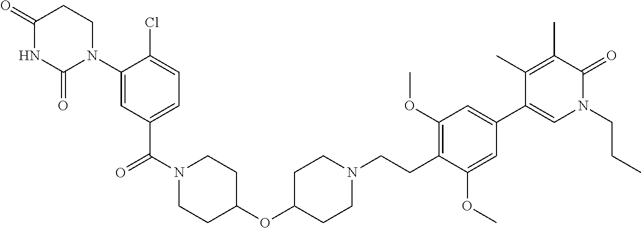 [SMILES: CCCn1cc(-c2cc(OC)c(CCN3CCC(OC4CCN(C(=O)c5ccc(Cl)c(N6CCC(=O)NC6=O)c5)CC4)CC3)c(OC)c2)c(C)c(C)c1=O]

To a 25 ml round bottom flask were added 4-chloro-3-(2,4-dioxotetrahydropyrimidin-1(2H)-yl)benzoic acid (intermediate 24, 33 mg, 0.12 mmol), HATU (46 mg, 0.12 mmol) and DMF (4 ml). The RM was stirred at RT for 10 min, DIEA (134 μL, 0.12 mmol) and 5-(3,5-dimethoxy-4-(2-(4-(piperidin-4-yloxy)piperidin-1-yl)ethyl)phenyl)-3,4-dimethyl-1-propylpyridin-2(1H)-one hydrochloride salt (60 mg, 0.10 mmol) were added. The RM was stirred at RT for 1 h, the solvent removed and the residue purified by preparative HPLC on an Xtimate C18 column (250×21.2 mm, 10 μm) eluting with ACN (from 5% to 95%) in aq. NH₄HCO₃ (0.01 M), yielding the title compound as a solid (16 mg).

Method E: Rt=1.48 min; [M+H]⁺=763.

¹H NMR (500 MHz, DMSO) δ 10.53 (s, 1H), 7.64 (d, J=8.5 Hz, 1H), 7.58 (brs, 1H), 7.45 (s, 1H), 7.40 (dd, J=8.5, 2.0 Hz, 1H), 6.50 (s, 2H), 3.97 (m, 1H), 3.85 (t, J=7.5 Hz, 2H), 3.77 (m, 1H), 3.77 (s, 6H), 3.70 (m, 1H), 3.62 (m, 1H), 3.48-3.43 (m, 2H), 3.27-3.15 (m, 2H), 2.76-2.64 (m, 6H), 2.29 (m, 2H), 2.08 (m, 2H), 2.04 (s, 6H), 1.79 (s, 4H), 1.65 (m, 2H), 1.43 (m, 4H), 0.86 (t, J=7.5 Hz, 3H).

Compound B3: 1-(5-(44(1-(4-(2-butyl-1-oxo-1,2-dihydro-2,7-naphthyridin-4-yl)-2,5-dimethoxybenzyl)piperidin-4-yl)oxy)piperidine-1-carbonyl)-2-methoxyphenyl)dihydropyrimidine-2,4(1H,3H)-dione

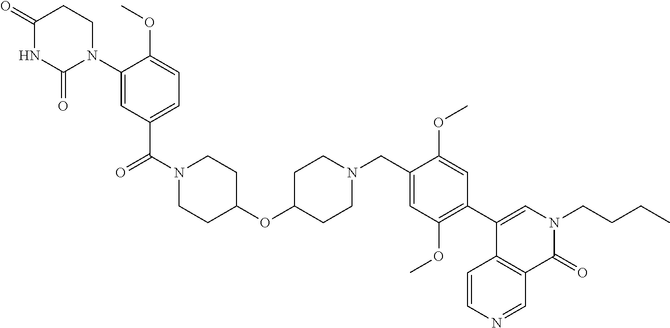 [SMILES: CCCCn1cc(-c2cc(OC)c(CN3CCC(OC4CCN(C(=O)c5ccc(OC)c(N6CCC(=O)NC6=O)c5)CC4)CC3)cc2OC)c2ccncc2c1=O]

### Step 1: tert-butyl 4-((1-(4-(2-butyl-1-oxo-1,2-dihydro-2,7-naphthyridin-4-yl)-2,5-dimethoxybenzyl)piperidin-4-yl)oxy)piperidine-1-carboxylate

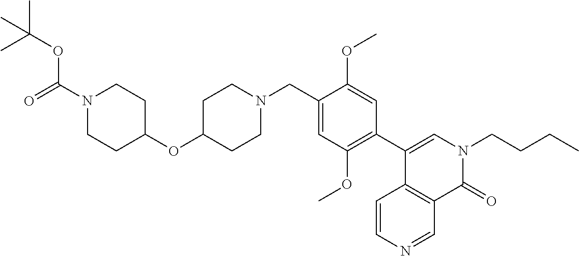 [SMILES: CCCCn1cc(-c2cc(OC)c(CN3CCC(OC4CCN(C(=O)OC(C)(C)C)CC4)CC3)cc2OC)c2ccncc2c1=O]

To a 50 ml round bottom flask were added 4-(2-butyl-1-oxo-1,2-dihydro-2,7-naphthyridin-4-yl)-2,5-dimethoxybenzaldehyde (intermediate 33, 329 mg, 0.9 mmol), tert-butyl 4-(piperidin-4-yloxy)piperidine-1-carboxylate (intermediate 1, 384 mg, 1.35 mmol), a solution of ZnCl₂ (1 M) in THE (1.5 ml, 1.5 mmol) and DMSO (5 ml). The RM was stirred at RT for 1 h, solid NaBH₃CN (227 mg, 3.6 mmol) and MeOH (0.5 ml) were added and the RM was stirred at RT for 16 h. The mixture was concentrated and the residue was purified by reversed phase chromatography on a Biotage Agela C18 column (120 g, spherical 20-35 μm, 100 Å) eluting with ACN (from 5% to 95%) in aq. NH₄HCO₃ (0.1%), yielding the title compound as a solid (154 mg).

Method H: Rt=2.45 min; [M+H]⁺=635.

### Step 2: 2-butyl-4-(2,5-dimethoxy-4-((4-(piperidin-4-yloxy)piperidin-1-yl)methyl)phenyl)-2,7-naphthyridin-1(2H)-one

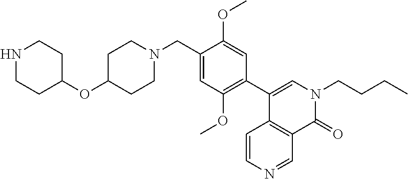 [SMILES: CCCCn1cc(-c2cc(OC)c(CN3CCC(OC4CCNCC4)CC3)cc2OC)c2ccncc2c1=O]

To a 50 ml round bottom flask were added tert-butyl 4-((1-(4-(2-butyl-1-oxo-1,2-dihydro-2,7-naphthyridin-4-yl)-2,5-dimethoxybenzyl)piperidin-4-yl)oxy)piperidine-1-carboxylate (154 mg, 0.24 mmol), DCM (5 ml) and a solution of HCl (4 M) in 1,4-dioxane (2 ml). The RM was stirred at RT for 1 h and evaporated to dryness, yielding the corresponding hydrochloride salt of the title compound as a solid (144 mg), which was used for the next step without further purification.

Method H: Rt=1.27 min; [M+H]⁺=535.

### Step 3: 1-(5-(4-((1-(4-(2-butyl-1-oxo-1,2-dihydro-2,7-naphthyridin-4-yl)-2,5-dimethoxybenzyl)piperidin-4-yl)oxy)piperidine-1-carbonyl)-2-methoxyphenyl)dihydropyrimidine-2,4(1H,3H)-dione

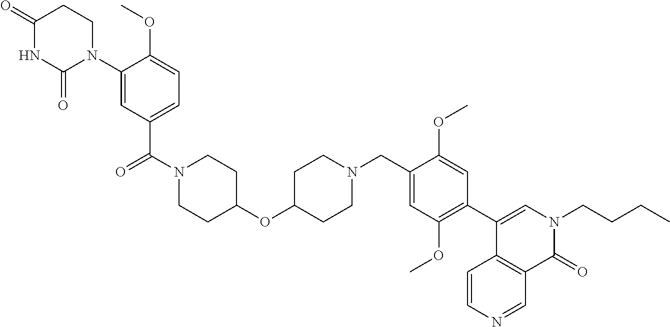 [SMILES: CCCCn1cc(-c2cc(OC)c(CN3CCC(OC4CCN(C(=O)c5ccc(OC)c(N6CCC(=O)NC6=O)c5)CC4)CC3)cc2OC)c2ccncc2c1=O]

To a 50 ml round bottom flask were added 2-butyl-4-(2,5-dimethoxy-4-((4-(piperidin-4-yloxy)piperidin-1-yl)methyl)phenyl)-2,7-naphthyridin-1(2H)-one hydrochloride (144 mg, 0.27 mmol), perfluorophenyl 3-(2,4-dioxotetrahydropyrimidin-1(2H)-yl)-4-methoxybenzoate (intermediate 26,116 mg, 0.27 mmol), DMF (4 ml) and DIPEA (139 mg, 1.1 mmol). The RM was stirred at RT for 1 h, the solvent was removed and the residue was purified by preparative HPLC on an Xtimate C18 column (250×21.2 mm, 10 μm) eluting with ACN (from 5% to 95%) in aq. NH₄HCO₃ (0.01 M), yielding the title compound as a solid (49 mg).

Method H: Rt=1.83 min; [M+H]⁺=781.

¹H NMR (500 MHz, DMSO) δ 10.34 (s, 1H), 9.41 (s, 1H), 8.85 (d, J=5.6 Hz, 1H), 7.77-7.68 (M, 1H), 7.38 (dd, J=8.4, 2.0 Hz, 1H), 7.34 (d, J=2.0 Hz, 1H), 7.15 (d, J=8.6 Hz, 2H), 7.05 (d, J=5.5 Hz, 1H), 6.92 (s, 1H), 4.09-3.96 (m, 2H), 3.95-3.35 (m, 17H), 3.32-3.11 (m, 4H), 2.89-2.59 (m, 3H), 2.20-2.12 (m, 1H), 1.96-1.74 (m, 4H), 1.73-1.68 (m, 2H), 1.60-1.23 (m, 6H), 0.93 (t, J=7.4 Hz, 3H).

### Compound B4: 1-(5-(4-((1-(4-(2-butyl-1-oxo-1,2-dihydro-2,7-naphthyridin-4-yl)-2,6-dimethoxyphenethyl)piperidin-4-yl)oxy)piperidine-1-carbonyl)-2-chlorophenyl)dihydropyrimidine-2,4(1H,3H)-dione

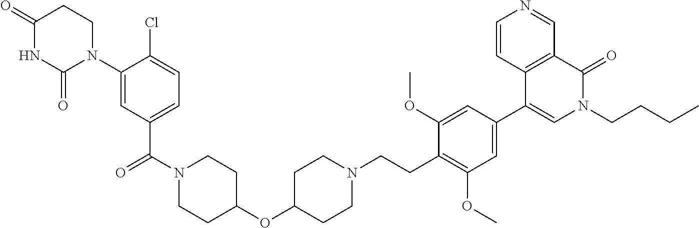 [SMILES: CCCCn1cc(-c2cc(OC)c(CCN3CCC(OC4CCN(C(=O)c5ccc(Cl)c(N6CCC(=O)NC6=O)c5)CC4)CC3)c(OC)c2)c2ccncc2c1=O]

### Step 1: 2-(3,5-dimethoxy-4-(2-methoxyvinyl)phenyl)-4,4,5,5-tetramethyl-1,3,2-dioxaborolane

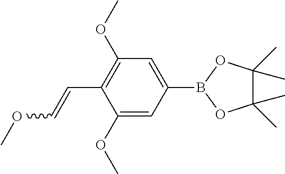

To a 250 ml round bottom flask were added (methoxymethyl)triphenylphosphonium chloride (2.06 g, 6 mmol), t-BuOK (897 mg, 8 mmol) and THE (50 ml). The RM was stirred at 0° C. for 30 min, solid 2,6-dimethoxy-4-(4,4,5,5-tetramethyl-1,3,2-dioxaborolan-2-yl)benzaldehyde (intermediate 7, 584 mg, 2 mmol) was added and the RM was stirred at 0° C. for 5 h. The mixture was concentrated and the residue was purified by chromatography on silica gel eluting with EtOAc (from 0% to 100%) in PE, yielding the title compound as a solid (310 mg).

Method H: Rt=2.179 min; [M+H]⁺=321.

### Step 2: 2-butyl-4-(3,5-dimethoxy-4-(2-methoxyvinyl)phenyl)-2,7-naphthyridin-1(2H)-one

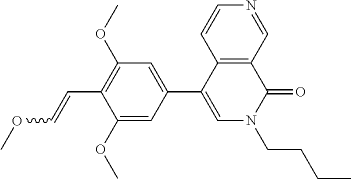 [SMILES: CCCCn1cc(-c2cc(OC)c(C=COC)c(OC)c2)c2ccncc2c1=O]

To a 50 ml round bottom flask were added under an argon atmosphere 2-(3,5-dimethoxy-4-(2-methoxyvinyl)phenyl)-4,4,5,5-tetramethyl-1,3,2-dioxaborolane (310 mg, 0.97 mmol), Na₂CO₃ (256 mg, 2.46 mmol), 1,4-dioxane (4 ml), water (1 ml), 4-bromo-2-butyl-2,7-naphthyridin-1(2H)-one (intermediate 32, 226 mg, 0.81 mmol) and PdCl₂(dppf) (29 mg, 0.04 mmol). The RM was stirred at 100° C. for 2 h. The solvents were removed and the residue was purified by chromatography on silica gel eluting with EtOAc (from 0% to 100%) in PE, yielding the title compound as a solid (270 mg).

Method H: Rt=2.005 min; [M+H]⁺=395.

### Step 3: 2-(4-(2-butyl-1-oxo-1,2-dihydro-2,7-naphthyridin-4-yl)-2,6-dimethoxyphenyl)acetaldehyde

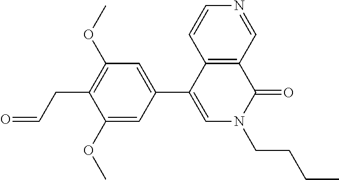 [SMILES: CCCCn1cc(-c2cc(OC)c(CC=O)c(OC)c2)c2ccncc2c1=O]

To a 50 ml round bottom flask were added 2-butyl-4-(3,5-dimethoxy-4-(2-methoxyvinyl)phenyl)-2,7-naphthyridin-1(2H)-one (270 mg, 0.68 mmol), acetone (10 ml) and an aq. solution of HCl (2 M, 2 ml). The RM was stirred at 65° C. for 5 h and concentrated, yielding the corresponding hydrochloride salt of the title compound as an oil (200 mg), which was used for the next step without further purification.

Method H: Rt=1.981 min; [M+H]⁺=381.

### Step 4: 1-(5-(4-((1-(4-(2-butyl-1-oxo-1,2-dihydro-2,7-naphthyridin-4-yl)-2,6-dimethoxyphenethyl)piperidin-4-yl)oxy)piperidine-1-carbonyl)-2-chlorophenyl)dihydropyrimidine-2,4(1H,3H)-dione

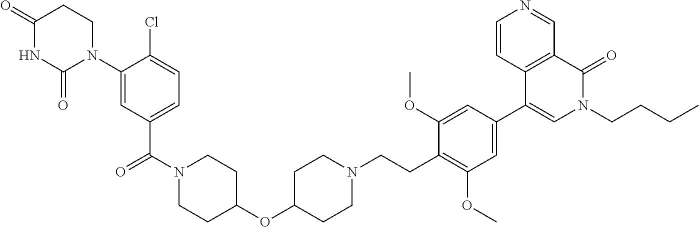 [SMILES: CCCCn1cc(-c2cc(OC)c(CCN3CCC(OC4CCN(C(=O)c5ccc(Cl)c(N6CCC(=O)NC6=O)c5)CC4)CC3)c(OC)c2)c2ccncc2c1=O]

To a 50 ml round bottom flask were added 1-(2-chloro-5-(4-(piperidin-4-yloxy)piperidine-1-carbonyl)phenyl)dihydropyrimidine-2,4(1H,3H)-dione hydrochloride (intermediate 28, 180 mg, 0.38 mmol), DMSO (5 ml) and DIPEA (148 mg, 1.14 mmol). The RM was stirred at RT for 12 min, solid 2-(4-(2-butyl-1-oxo-1,2-dihydro-2,7-naphthyridin-4-yl)-2,6-dimethoxyphenyl)acetaldehyde (200 mg, 0.56 mmol) and a solution of ZnCl₂ (1 M) in THE (0.57 ml, 0.57 mmol) were added and the RM was stirred at RT for 30 min. Solid NaBH₃CN (96 mg, 1.52 mmol) was added and stirring was continued at RT for 30 min. MeOH (5 ml) was added and the RM was stirred at RT for 2 h. The mixture was filtered, the filtrate was concentrated and the residue was purified by preparative HPLC on an XBridge C18 column (250×21.2 mm, 10 μm) eluting with ACN (from 5% to 95%) in aq. NH₄HCO₃ (0.01 M), yielding the title compound as a solid (45 mg).

Method H: Rt=2.131 min; [M+H]⁺=799.

¹H NMR (500 MHz, DMSO) δ 10.517 (s, 1H), 9.440 (s, 1H), 8.712 (d, J=5 Hz, 1H), 7.819 (s, 1H), 7.641 (d, J=8 Hz, 1H), 7.579-7.552 (m, 2H), 7.407 (d, J=8 Hz, 1H), 6.696 (s, 2H), 4.053-3.953 (m, 3H), 3.810-3.436 (m, 11H), 3.262-3.183 (m, 2H), 2.759-2.697 (m, 6H), 2.361-2.332 (m, 2H), 2.160-2.067 (m, 2H), 1.826-1.687 (m, 6H), 1.452-1.310 (m, 6H), 0.929 (t, J=7.3 Hz, 3H).

### Compound B5: 1-(5-(4-((4-(4-(2-butyl-1-oxo-1,2-dihydro-2,7-naphthyridin-4-yl)-2-methoxyphenoxy)piperidin-1-yl)methyl)piperidine-1-carbonyl)-2-methoxyphenyl)dihydropyrimidine-2,4(1H,3H)-dione

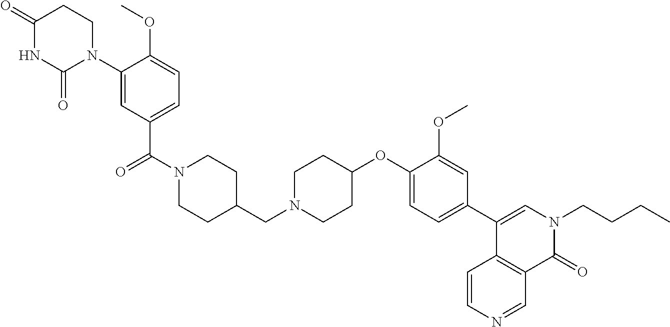 [SMILES: CCCCn1cc(-c2ccc(OC3CCN(CC4CCN(C(=O)c5ccc(OC)c(N6CCC(=O)NC6=O)c5)CC4)CC3)c(OC)c2)c2ccncc2c1=O]

### Step 1: tert-butyl 4-(4-(2-butyl-1-oxo-1,2-dihydro-2,7-naphthyridin-4-yl)-2-methoxyphenoxy)piperidine-1-carboxylate

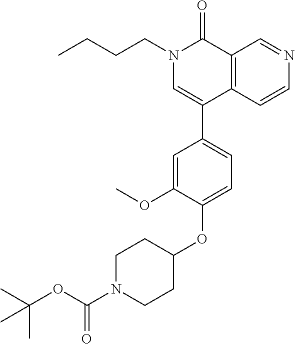 [SMILES: CCCCn1cc(-c2ccc(OC3CCN(C(=O)OC(C)(C)C)CC3)c(OC)c2)c2ccncc2c1=O]

To a 50 ml round bottom flask were added under an argin atmosphere tert-butyl 4-(2-methoxy-4-(4,4,5,5-tetramethyl-1,3,2-dioxaborolan-2-yl)phenoxy)piperidine-1-carboxylate (intermediate 16, 390 mg, 0.9 mmol), 4-bromo-2-butyl-2,7-naphthyridin-1(2H)-one (intermediate 32, 252 mg, 0.9 mmol), Na₂CO₃ (238 mg, 2.25 mmol), ACN (12 ml), water (3 ml) and PdCl₂(dppf) (66 mg, 0.09 mmol). The RM was stirred at 100° C. for 2 h, the solvents were removed and the residue was purified by chromatography on silica gel eluting with EtOAc (from 0% to 80%) in PE, yielding the title compound as a solid (470 mg).

Method E: Rt=1.86 min; [M+H]⁺=508.

### Step 2: 2-butyl-4-(3-methoxy-4-(piperidin-4-yloxy)phenyl)-2,7-naphthyridin-1(2H)-one

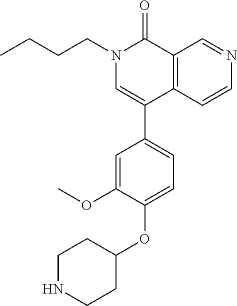 [SMILES: CCCCn1cc(-c2ccc(OC3CCNCC3)c(OC)c2)c2ccncc2c1=O]

To a 50 ml round bottom flask were added tert-butyl 4-(4-(2-butyl-1-oxo-1,2-dihydro-2,7-naphthyridin-4-yl)-2-methoxyphenoxy)piperidine-1-carboxylate (470 mg, 0.927 mmol), DCM (6 ml), MeOH (1 ml) and a solution of HCl (4 M) in 1,4-dioxane (3 ml). The RM was stirred at RT for 1 h and evaporated to dryness, yielding the corresponding hydrochloride salt of the title compound as a solid (450 mg), which was used for the next step without further purification.

Method E: Rt=1.31 min; [M+H]⁺=408.

### Step 3: tert-butyl 4-((4-(4-(2-butyl-1-oxo-1,2-dihydro-2,7-naphthyridin-4-yl)-2-methoxyphenoxy)piperidin-1-yl)methyl)piperidine-1-carboxylate

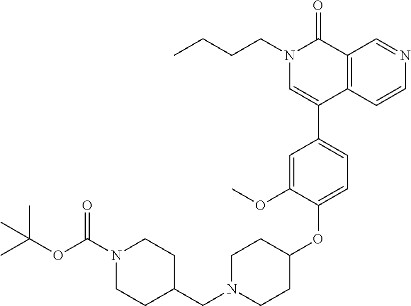 [SMILES: CCCCn1cc(-c2ccc(OC3CCN(CC4CCN(C(=O)OC(C)(C)C)CC4)CC3)c(OC)c2)c2ccncc2c1=O]

To a 50 ml round bottom flask were added 2-butyl-4-(3-methoxy-4-(piperidin-4-yloxy)phenyl)-2,7-naphthyridin-1(2H)-one hydrochloride (400 mg, 0.824 mmol), tert-butyl 4-formylpiperidine-1-carboxylate (193 mg, 0.906 mmol), K₂CO₃ (277 mg, 1.648 mmol), a solution of ZnCl₂ (1 M) in THE (1.07 ml, 1.07 mmol) and DMSO (4 ml). The RM was stirred at RT for 30 min, solid NaBH₃CN (207 mg, 3.29 mmol) and MeOH (1 ml) were added and the RM was stirred at RT for 2 h. The mixture was added into water (10 ml), extracted with EtOAc (3×20 ml), the combined organic phases were washed with brine, dried over Na₂SO₄ and the residue was purified by chromatography on silica gel eluting with EtOAc (from 0% to 100%) in PE, yielding the title compound as a solid (275 mg).

Method G: Rt=2.28 min; [M+H]⁺=605.

### Step 4: 2-butyl-4-(3-methoxy-4-((1-(piperidin-4-ylmethyl)piperidin-4-yl)oxy)phenyl)-2,7-naphthyridin-1(2H)-one

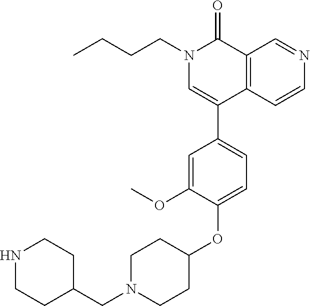 [SMILES: CCCCn1cc(-c2ccc(OC3CCN(CC4CCNCC4)CC3)c(OC)c2)c2ccncc2c1=O]

To a 50 ml round bottom flask were added tert-butyl 4-((4-(4-(2-butyl-1-oxo-1,2-dihydro-2,7-naphthyridin-4-yl)-2-methoxyphenoxy)piperidin-1-yl)methyl)piperidine-1-carboxylate (275 mg, 0.455 mmol), DCM (4 ml), MeOH (1 ml) and a solution of HCl (4 M) in 1,4-dioxane (2 ml). The RM was stirred at RT for 1 h and evaporated to dryness, yielding the corresponding hydrochloride salt of the title compound as a solid (300 mg), which was used for the next step without further purification.

Method E: Rt=1.29 min; [M+H]⁺=505.

### Step 5: 1-(5-(4-((4-(4-(2-butyl-1-oxo-1,2-dihydro-2,7-naphthyridin-4-yl)-2-methoxyphenoxy)piperidin-1-yl)methyl)piperidine-1-carbonyl)-2-methoxyphenyl)dihydropyrimidine-2,4(1H,3H)-dione

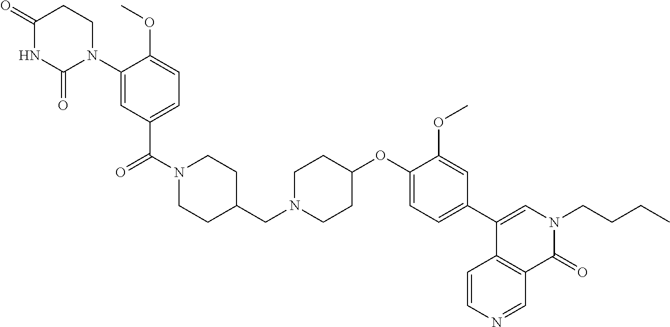 [SMILES: CCCCn1cc(-c2ccc(OC3CCN(CC4CCN(C(=O)c5ccc(OC)c(N6CCC(=O)NC6=O)c5)CC4)CC3)c(OC)c2)c2ccncc2c1=O]

To a 50 ml round bottom flask were added 2-butyl-4-(3-methoxy-4-((1-(piperidin-4-ylmethyl)piperidin-4-yl)oxy)phenyl)-2,7-naphthyridin-1(2H)-one hydrochloride (174 mg, 0.26 mmol), perfluorophenyl 3-(2,4-dioxotetrahydropyrimidin-1(2H)-yl)-4-methoxybenzoate (intermediate 26, 112 mg, 0.26 mmol), DIEA (168 mg, 1.3 mmol) and DMF (3 ml). The RM was stirred at RT for 2 h, the solvent was removed and the residue was purified by preparative HPLC on an XBridge C18 column (250×21.2 mm, 10 μm) eluting with ACN (from 5% to 80%) in aq. NH₄HCO₃ (0.01 M), yielding the title compound as a solid (122 mg).

Method G: Rt=1.82 min; [M+H]⁺=751.

¹H NMR (500 MHz, DMSO) δ 10.34 (s, 1H), 9.43 (s, 1H), 8.71 (d, J=5.6 Hz, 1H), 7.77 (s, 1H), 7.49 (d, J=5.7 Hz, 1H), 7.37 (dd, J=8.4, 1.9 Hz 1H), 7.32 (d, J=1.9 Hz, 1H), 7.19-7.08 (m, 2H), 7.05 (d, J=1.7 Hz, 1H), 6.94 (dd, J=8.2, 1.7 Hz 1H), 4.39-4.28 (m, 2H), 4.03 (t, J=7.2 Hz, 2H), 3.87-3.77 (m, 6H), 3.60 (t, J=7.2 Hz, 2H), 3.30-3.35 (m, 2H), 2.95-3.15 (m, 1H), 2.70-2.67 (m, 4H), 2.19-2.13 (m, 4H), 1.98-1.91 (m, 2H), 1.85-1.60 (m, 7H), 1.40-1.29 (m, 2H), 1.13-1.00 (m, 2H), 0.92 (t, J=7.4 Hz, 3H).

### Compound B6: 1-(5-(4-((4-(4-(2-butyl-1-oxo-1,2-dihydro-2,7-naphthyridin-4-yl)-2-methoxyphenoxy)piperidin-1-yl)methyl)piperidine-1-carbonyl)-2-chlorophenyl)dihydropyrimidine-2,4(1H,3H)-dione

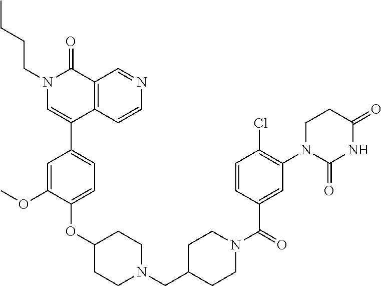 [SMILES: CCCCn1cc(-c2ccc(OC3CCN(CC4CCN(C(=O)c5ccc(Cl)c(N6CCC(=O)NC6=O)c5)CC4)CC3)c(OC)c2)c2ccncc2c1=O]

To a 50 ml round bottom flask were added HATU (80 mg, 0.210 mmol), 4-chloro-3-(2,4-dioxotetrahydropyrimidin-1(2H)-yl)benzoic acid (intermediate 24, 52 mg, 0.194 mmol), DIPEA (0.100 ml, 0.573 mmol) and DMF (1 ml). The mixture was stirred at RT for 30 min. A solution of 2-butyl-4-(3-methoxy-4-((1-(piperidin-4-ylmethyl)piperidin-4-yl)oxy)phenyl)-2,7-naphthyridin-1(2H)-one TFA salt (intermediate 35, 135 mg, 0.175 mmol) and DIPEA (0.100 ml, 0.573 mmol) in DMF (1.5 ml) was added and the RM was stirred at RT for 2 h. The solvent was removed and the residue was purified by reversed phase chromatography on a REDISEP® Gold HP C18 column (15.5 g) eluting with ACN (from 2% to 100%) in aq. NH₄HCO₃ (0.1) yielding the title compound (88 mg) as a solid.

Method L: Rt=3.32 min; [M+H]⁺=755.

¹H NMR (600 MHz, DMSO-d₆) δ 10.54 (s, 1H), 9.42 (s, 1H), 8.69 (m, 1H), 7.87-7.28 (m, 5H), 7.18-6.80 (m, 3H), 4.65-3.47 (m, 11H), 3.15-2.60 (m, 6H), 2.31-1.52 (m, 12H), 1.33 (m, 2H), 1.16-1.00 (m, 2H), 0.91 (m, 3H).

### Compound B7: 1-(5-(4-((4-((4-(2-butyl-1-oxo-1,2-dihydro-2,7-naphthyridin-4-yl)-2,6-dimethoxyphenoxy)methyl)piperidin-1-yl)methyl)piperidine-1-carbonyl)-2-methoxyphenyl)dihydropyrimidine-2,4(1H,3H)-dione

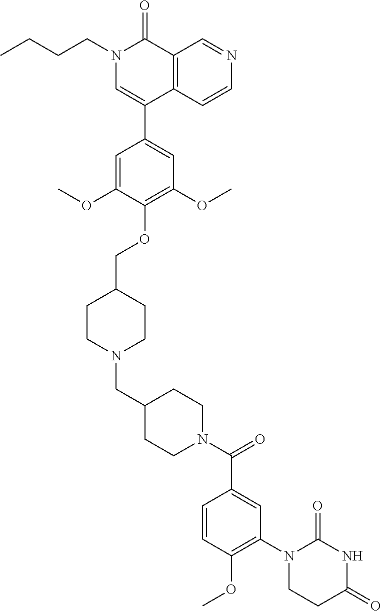 [SMILES: CCCCn1cc(-c2cc(OC)c(OCC3CCN(CC4CCN(C(=O)c5ccc(OC)c(N6CCC(=O)NC6=O)c5)CC4)CC3)c(OC)c2)c2ccncc2c1=O]

To a 50 ml round bottom flask were added HATU (47 mg, 0.124 mmol), 3-(2,4-dioxotetrahydropyrimidin-1(2H)-yl)-4-methoxybenzoic acid (intermediate 25, 30 mg, 0.113 mmol), DIPEA (0.100 ml, 0.573 mmol) and DMF (0.5 ml). The mixture was stirred at RT for 30 min. A solution of 2-butyl-4-(3,5-dimethoxy-4-((1-(piperidin-4-ylmethyl)piperidin-4-yl)methoxy)phenyl)-2,7-naphthyridin-1(2H)-one TFA salt (intermediate 36, 87 mg, 0.103 mmol) and DIPEA (0.100 ml, 0.573 mmol) in DMF (1 ml) was added and the RM was stirred at RT for 2 h. The solvent was removed and the residue was purified by reversed phase chromatography on a REDISEP® Gold HP C18 column (15.5 g) eluting with ACN (from 2% to 100%) in aq. NH₄HCO₃ (0.1%), yielding the title compound (52 mg) as a solid.

Method L: Rt=3.65 min; [M+H]⁺=795.

¹H NMR (400 MHz, DMSO-d₆) δ 10.35 (s, 1H), 9.45 (s, 1H), 8.73 (d, J=5.7 Hz, 1H), 7.82 (s, 1H), 7.56 (d, J=5.7 Hz, 1H), 7.38 (dd, J=8.4, 2.2 Hz, 1H), 7.34 (d, J=2.1 Hz, 1H), 7.17 (d, J=8.6 Hz, 1H), 6.76 (s, 2H), 4.05 (t, J=7.4 Hz, 2H), 3.87 (s, 3H), 3.82 (s, 6H), 3.77 (d, J=6.4 Hz, 2H), 3.62 (t, J=6.7 Hz, 2H), 2.87 (m, 4H), 2.70 (t, J=6.6 Hz, 2H), 2.17 (d, J=6.9 Hz, 2H), 1.81 (m, 11H), 1.46-1.02 (m, 7H), 0.94 (t, J=7.3 Hz, 3H).

### Compound B8: 1-(5-(4-((4-((4-(2-butyl-1-oxo-1,2-dihydro-2,7-naphthyridin-4-yl)-2,6-dimethoxyphenoxy)methyl)piperidin-1-yl)methyl)piperidine-1-carbonyl)-2-chlorophenyl)dihydropyrimidine-2,4(1H,3H)-dione

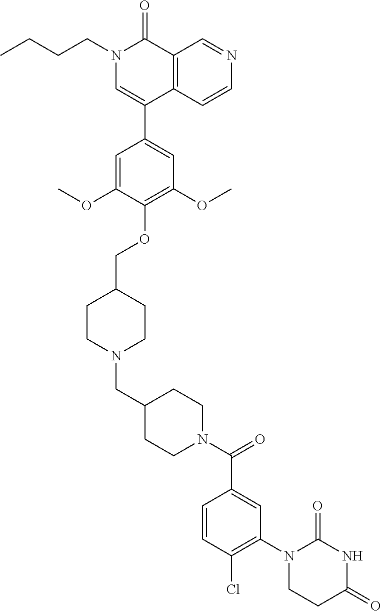 [SMILES: CCCCn1cc(-c2cc(OC)c(OCC3CCN(CC4CCN(C(=O)c5ccc(Cl)c(N6CCC(=O)NC6=O)c5)CC4)CC3)c(OC)c2)c2ccncc2c1=O]

To a 50 ml round bottom flask were added HATU (47 mg, 0.124 mmol), 4-chloro-3-(2,4-dioxotetrahydropyrimidin-1(2H)-yl)benzoic acid (intermediate 24, 31 mg, 0.115 mmol), DIPEA (0.054 ml, 0.309 mmol) and DMF (1 ml). The mixture was stirred at RT for 30 min, a solution of 2-butyl-4-(3,5-dimethoxy-4-((1-(piperidin-4-ylmethyl)piperidin-4-yl)methoxy)phenyl)-2,7-naphthyridin-1(2H)-one TFA salt (intermediate 36, 87 mg, 0.103 mmol) and DIPEA (0.054 ml, 0.309 mmol) in DMF (1 ml) was added and the RM was stirred at RT for 2 h. The solvent was removed and the residue was purified by reversed phase chromatography on a REDISEP® Gold HP C18 column (15.5 g) eluting with ACN (from 2% to 100%) in aq. NH₄HCO₃ (0.1%) yielding the title compound (58 mg) as a solid.

Method L: Rt=3.77 min; [M+H]⁺=799.

¹H NMR (400 MHz, DMSO-d₆) δ 10.53 (s, 1H), 9.46 (s, 1H), 8.74 (d, J=5.6 Hz, 1H), 7.83 (s, 1H), 7.66 (d, J=8.2 Hz, 1H), 7.57 (m, 2H), 7.41 (m, 1H), 6.76 (s, 2H), 4.47 (m, 1H), 4.05 (t, J=7.4 Hz, 2H), 3.82 (s, 6H), 3.77 (m, 3H), 3.72-3.54 (m, 2H), 3.07 (m, 1H), 2.87 (m, 2H), 2.82-2.72 (m, 3H), 2.16 (m, 2H), 1.96-1.60 (m, 8H), 1.43-1.05 (m, 7H), 0.95 (t, J=7.3 Hz, 3H).

### Compound B9: 1-(5-(4-((4-(4-(2-butyl-1-oxo-1,2-dihydro-2,7-naphthyridin-4-yl)-2,6-dimethoxyphenoxy)piperidin-1-yl)methyl)piperidine-1-carbonyl)-2-chlorophenyl)dihydropyrimidine-2,4(1H,3H)-dione

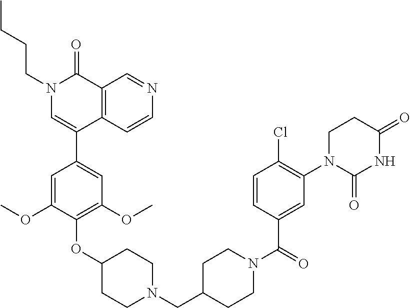 [SMILES: CCCCn1cc(-c2cc(OC)c(OC3CCN(CC4CCN(C(=O)c5ccc(Cl)c(N6CCC(=O)NC6=O)c5)CC4)CC3)c(OC)c2)c2ccncc2c1=O]

To a 50 ml round bottom flask were added HATU (60 mg, 0.158 mmol), 4-chloro-3-(2,4-dioxotetrahydropyrimidin-1(2H)-yl)benzoic acid (intermediate 24, 40 mg, 0.149 mmol), DIPEA (0.100 ml, 0.573 mmol) and DMF (1 ml). The mixture was stirred at RT for 30 min, a solution of 2-butyl-4-(3,5-dimethoxy-4-((1-(piperidin-4-ylmethyl)piperidin-4-yl)oxy)phenyl)-2,7-naphthyridin-1(2H)-one TFA salt (intermediate 37, 122 mg, 0.128 mmol) and DIPEA (0.100 ml, 0.573 mmol) in DMF (1.5 ml) was added and the RM was stirred at RT for 2 h. The solvent was removed and the residue was purified by reversed phase chromatography on a REDISEP® Gold HP C18 column (15.5 g) eluting with ACN (from 2% to 10%) in aq. NH₄HCO₃ (0.1) yielding the title compound (66 mg) as a solid.

Method L: Rt=2.89 min; [M+H]⁺=786.

¹H NMR (400 MHz, DMSO-d₆) δ 10.54 (s, 1H), 9.46 (s, 1H), 8.74 (d, J=5.6 Hz, 1H), 7.84 (s, 1H), 7.67 (d, J=8.2 Hz, 1H), 7.57 (m, 2H), 7.41 (d, J=8.2 Hz, 1H), 6.77 (s, 2H), 4.47 (m, 1H), 4.06 (m, 3H), 3.83 (m, 7H), 3.72-3.52 (m, 3H), 3.18-2.96 (m, 1H), 2.90-2.66 (m, 4H), 2.13 (m, 4H), 1.95-1.59 (m, 9H), 1.36 (m, 2H), 1.20-1.03 (m, 2H), 0.95 (t, J=7.4 Hz, 3H).

### Compound B10: 1-(5-(4-((4-(4-(2-butyl-1-oxo-1,2-dihydro-2,7-naphthyridin-4-yl)-2,6-dimethoxyphenoxy)piperidin-1-yl)methyl)piperidine-1-carbonyl)-2-methoxyphenyl)dihydropyrimidine-2,4(1H,3H)-dione

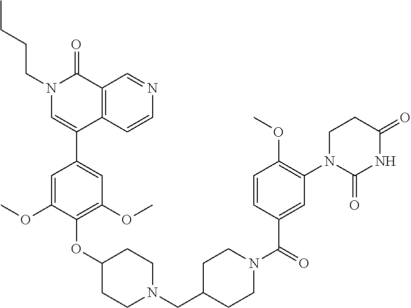 [SMILES: CCCCn1cc(-c2cc(OC)c(OC3CCN(CC4CCN(C(=O)c5ccc(OC)c(N6CCC(=O)NC6=O)c5)CC4)CC3)c(OC)c2)c2ccncc2c1=O]

To a 50 ml round bottom flask were added HATU (68 mg, 0.179 mmol), 3-(2,4-dioxotetrahydropyrimidin-1(2H)-yl)-4-methoxybenzoic acid (intermediate 25, 45 mg, 0.170 mmol), DIPEA (0.100 ml, 0.573 mmol) and DMF (1.5 ml). The mixture was stirred at RT for 30 min, a solution of 2-butyl-4-(3,5-dimethoxy-4-((1-(piperidin-4-ylmethyl)piperidin-4-yl)oxy)phenyl)-2,7-naphthyridin-1(2H)-one TFA salt (intermediate 37, 122 mg, 0.147 mmol) and DIPEA (0.100 ml, 0.573 mmol) in DMF (1.5 ml) was added and the RM was stirred at RT for 2 h. The solvent was removed and the residue was purified by reversed phase chromatography on a REDISEP® Gold HP C18 column (15.5 g) eluting with ACN (from 2% to 100%) in aq. NH₄HCO₃ (0.1%), yielding the title compound (88 mg) as a solid.

Method L: Rt=2.78 min; [M−H]⁺=779.

¹H NMR (400 MHz, DMSO-d₆) δ 10.36 (s, 1H), 9.46 (s, 1H), 8.74 (d, J=5.6 Hz, 1H), 7.84 (s, 1H), 7.57 (d, J=5.7 Hz, 1H), 7.39 (d, J=8.6 Hz, 1H), 7.34 (s, 1H), 7.18 (d, J=8.4 Hz, 1H), 6.76 (s, 2H), 4.41 (m, 1H), 4.05 (m, 4H), 3.85 (m, 8H), 3.62 (t, J=6.7 Hz, 2H), 2.73 (m, 6H), 2.13 (m, 5H), 1.95-1.58 (m, 9H), 1.37 (m, 2H), 1.08 (m, 2H), 0.95 (t, J=7.2 Hz, 3H).

### Synthesis of Compound C1: (1-((1-(2-(4-(1-(2,5-Dimethoxy-4-(2-methyl-1-oxo-1,2-dihydro-2,7-naphthyridin-4-yl)benzyl)piperidin-4-yloxy)piperidin-1-yl)ethyl)-2-oxo-1,2-dihydropyridin-3-yl)methyl)dihydropyrimidine-2,4(1H,3H)-dione

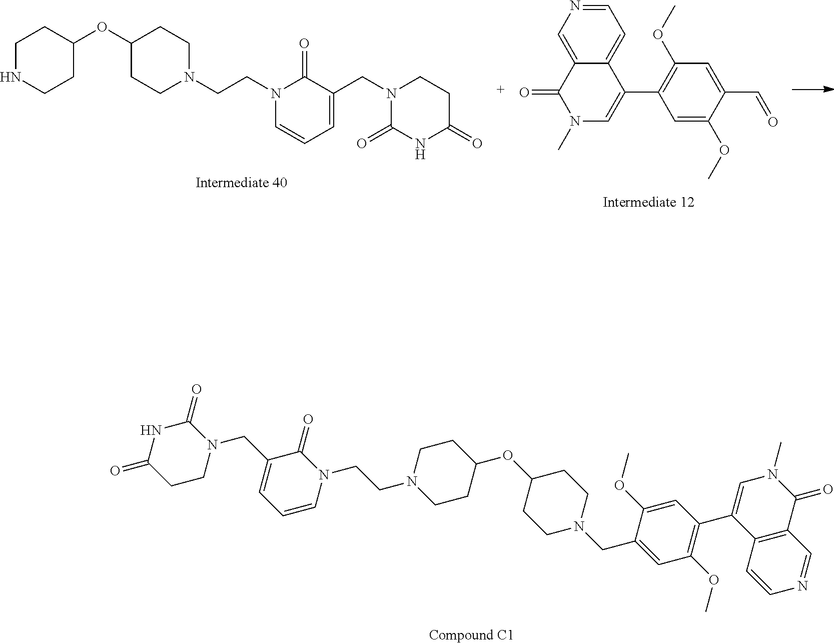 [SMILES: O=C1CCN(Cc2cccn(CCN3CCC(OC4CCNCC4)CC3)c2=O)C(=O)N1 ; COc1cc(-c2cn(C)c(=O)c3cnccc23)c(OC)cc1C=O ; COc1cc(-c2cn(C)c(=O)c3cnccc23)c(OC)cc1CN1CCC(OC2CCN(CCn3cccc(CN4CCC(=O)NC4=O)c3=O)CC2)CC1]

To a 250 mL round bottom flask was added 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione (Intermediate 40, 1.6, 3.71 mmol), 2,5-dimethoxy-4-(2-methyl-1-oxo-1,2-dihydro-2,7-naphthyridin-4-yl)benzaldehyde (Intermediate 12, 1.2 g, 3.71 mmol), a solution of ZnCl₂ (1 M) in THE (5.6 mL, 5.6 mmol), and DMSO (25 mL) and the resulting mixture was stirred at RT for 2 h. Solid NaBH₃CN (303 mg, 4.82 mmol) and MeOH (5 mL) were added and stirring was continued at RT for 48 h. MeOH (200 mL) was added and the RM was filtered, the filtrate was concentrated, and the residue purified by preparative HPLC on an XBridge C18 column (250×21.2 mm, 10 m) eluting with 500 to 80% ACN in aq. NH₄HCO₃ (0.01 M) to provide the title compound as a solid (802 mg).

LC-MS Method C: Rt=1.73 min; [M+H]⁺=740.

¹H NMR (500 MHz, DMSO-d₆) δ 10.15 (s, 1H), 9.40 (d, J=0.6 Hz, 1H), 8.64 (d, J=5.6 Hz, 1H), 7.74 (s, 1H), 7.58 (dd, J=6.7, 1.8 Hz, 1H), 7.27 (dd, J=6.8, 1.6 Hz, 1H), 7.14 (s, 1H), 7.03 (dd, J=5.6, 0.6 Hz, 1H), 6.91 (s, 1H), 6.20 (t, J=6.8 Hz, 1H), 4.26 (s, 2H), 3.99 (t, J=6.4 Hz, 2H), 3.74 (s, 3H), 3.63 (s, 3H), 3.57 (s, 3H), 3.50 (d, J=1.4 Hz, 2H), 3.41 (t, J=6.8 Hz, 4H), 2.74-2.72 (m, 4H), 2.57 (t, J=6.8 Hz, 2H), 2.54-2.51 (m, 2H), 2.16-2.09 (m, 4H), 1.82-1.74 (m, 4H), 1.51-1.31 (m, 4H).

### Synthesis of Compound C2: (1-((1-(2-(4-((1-(2,6-dimethoxy-4-(2-methyl-1-oxo-1,2-dihydro-2,7-naphthyridin-4-yl)benzyl)piperidin-4-yl)oxy)piperidin-1-yl)ethyl)-2-oxo-1,2-dihydropyridin-3-yl)methyl)dihydropyrimidine-2,4(1H,3H)-dione)

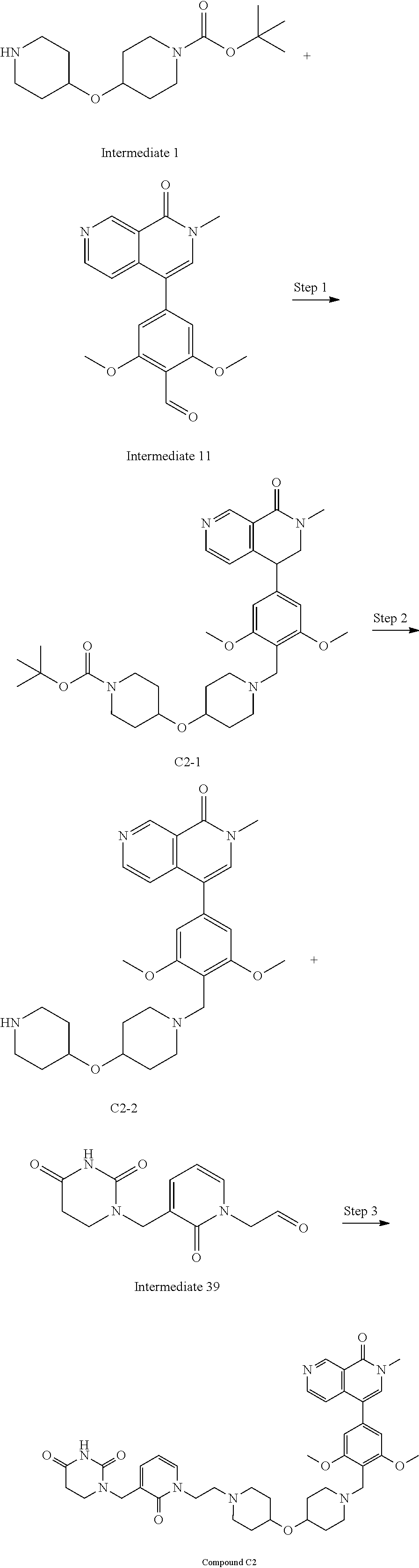

### Step 1: tert-butyl 4-((1-(2,6-dimethoxy-4-(2-methyl-1-oxo-1,2-dihydro-2,7-naphthyridin-4-yl)benzyl)piperidin-4-yl)oxy)piperidine-1-carboxylate (C2-1)

To a 25 mL round bottom flask was added tert-butyl 4-(piperidin-4-yloxy)piperidine-1-carboxylate (Intermediate 1, 250 mg, 0.88 mmol), 2,6-dimethoxy-4-(2-methyl-1-oxo-1,2-dihydro-2,7-naphthyridin-4-yl)benzaldehyde (Intermediate 11, 285 mg, 0.88 mmol), DMSO (5 mL), and a solution of ZnCl₂(1 M) in THE (1.76 mL, 1.76 mmol) and the resulting mixture was stirred at RT for 1 h. Solid NaBH₃CN (450 mg, 7 mmol) was added, and stirring was continued at RT for 16 h. The RM was added into brine (50 mL) and extracted with EtOAc (3×50 mL). The combined organic phases were dried over Na₂SO₄, filtered, and concentrated. The crude residue was purified by chromatography on silica gel eluting with 0% to 10% MeOH in DCM to provide the title compound 2-1 as a yellow solid (300 mg).

LC-MS Method E: Rt=1.471 min; [M+H]⁺=593.

### Step 2: 4-(3,5-dimethoxy-4-((4-(piperidin-4-yloxy)piperidin-1-yl)methyl)phenyl)-2-methyl-2,7-naphthyridin-1(2H)-one (C2-2)

To a 25 mL round bottom flask was added tert-butyl 4-((1-(2,6-dimethoxy-4-(2-methyl-1-oxo-1,2-dihydro-2,7-naphthyridin-4-yl)benzyl)piperidin-4-yl)oxy)piperidine-1-carboxylate (C2-1, 150 mg, 0.25 mmol), DCM (3 mL), MeOH (1 mL), and a solution of HCl (4 M) in 1,4-dioxane (10 mL) and the resulting mixture was stirred at RT for 3 h. The RM was evaporated to dryness to provide the hydrochloride salt of the title compound as a yellow solid (133 mg), which was used in the next step without further purification.

LC-MS Method E: Rt=1.152 min; [M+H]⁺=493.

### Step 3: 1-((1-(2-(4-((1-(2,6-dimethoxy-4-(2-methyl-1-oxo-1,2-dihydro-2,7-naphthyridin-4-yl)benzyl)piperidin-4-yl)oxy)piperidin-1-yl)ethyl)-2-oxo-1,2-dihydropyridin-3-yl)methyl)dihydropyrimidine-2,4(1H,3H)-dione (Compound C2)

To a 25 mL round bottom flask was added 2-(3-((2,4-dioxotetrahydropyrimidin-1(2H)-yl)methyl)-2-oxopyridin-1(2H)-yl)acetaldehyde (Intermediate 39, 71 mg, 0.25 mmol), 4-(3,5-dimethoxy-4-((4-(piperidin-4-yloxy)piperidin-1-yl)methyl)phenyl)-2-methyl-2,7-naphthyridin-1(2H)-one hydrochloride (C2-2, 133 mg, 0.25 mmol), DMSO (3 mL), and K₂CO₃ (42 mg, 0.3 mmol) and the RM was stirred at RT for 10 min. A solution of ZnCl₂(1.0 M) in THF (0.63 mL) was then added and stirring was continued at RT for 30 min. Solid NaBH₃CN (125 mg, 2 mmol) was added and the RM was stirred at RT for 16 h and concentrated. The crude residue was purified by reversed phase chromatography on a Biotage Agela C18 column (120 g, spherical 20-35 μm, 100 Å) eluting with 5% to 95% ACN in aq. NH₄HCO₃ (0.1%) to provide the title compound as a white solid (Compound C2, 38 mg).

LC-MS Method G: Rt=1.562 min; [M+H]⁺=740.

¹H NMR (500 MHz, DMSO-d₆) δ 10.16 (s, 1H), 9.44 (s 1H), 8.72 (d, J=5.7 Hz, 1H), 7.88 (s, 1H), 7.58 (d, J=5.8 Hz, 2H), 7.27 (d, J=5.8 Hz, 1H), 6.72 (s, 2H), 6.20 (t, J=6.8 Hz, 1H), 4.26 (s, 2H), 3.98 (t, J=6 Hz, 4 Hz, 2 Hz), 3.80 (s, 6H), 3.60 (s, 3H), 3.51 (s, 2H), 3.41-3.33 (m, 4H), 2.72 (s, 4H), 2.58-2.50 (m, 4H), 2.11 (m, 4H), 1.72 (s, 4H), 1.37-1.30 (m, 4H).

### Synthesis of Compound C3: (1-((1-(2-(4-((1-(2,5-dimethoxy-4-(2-methyl-1-oxo-1,2-dihydro-2,7-naphthyridin-4-yl)phenethyl)piperidin-4-yl)oxy)piperidin-1-yl)ethyl)-2-oxo-1,2-dihydropyridin-3-yl)methyl)dihydropyrimidine-2,4(1H,3H)-dione)

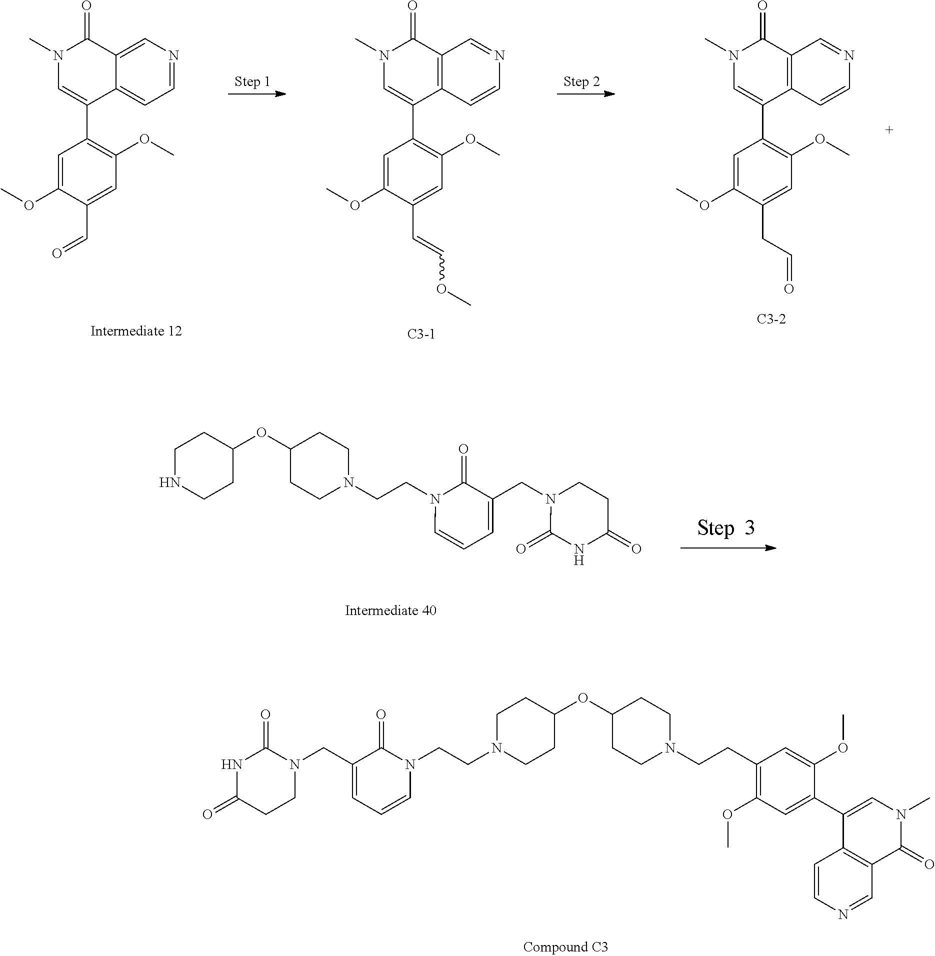

### Step 1: 4-(2,5-dimethoxy-4-(2-methoxyvinyl)phenyl)-2-methyl-2,7-naphthyridin-1(2H)-one (C3-1)

To a 100 mL round bottom flask cooled to 0° C. was containing (methoxymethyl)triphenylphosphonium chloride (648 mg, 2 mmol) and THE (15 mL) and was added solid t-BuOK (900 mg, 8 mmol) and the resulting mixture was stirred at 0° C. for 30 min. Solid 2,5-dimethoxy-4-(2-methyl-1-oxo-1,2-dihydro-2,7-naphthyridin-4-yl)benzaldehyde (Intermediate 12, 648 mg, 2 mmol) was added portionwise and stirring was continued at 70° C. for 16 h. The mixture was concentrated and then added into water (50 mL). The aqueous phase was extracted with EtOAc (3×50 mL) and the combined organic phases were dried over Na₂SO₄, filtered, and concentrated. The crude residue was purified by chromatography on silica gel eluting with 0% to 35% EtOAc in PE to provide the title compound C3-1 as a solid (1.3 g).

LC-MS Method G: Rt=1.82 min; [M+H]⁺=353.

### Step 2: 2-(2,5-dimethoxy-4-(2-methyl-1-oxo-1,2-dihydro-2,7-naphthyridin-4-yl)phenyl)acetaldehyde (C3-2)

To a 50 mL round bottom flask was added 4-(2,5-dimethoxy-4-(2-methoxyvinyl)phenyl)-2-methyl-2,7-naphthyridin-1(2H)-one (C3-1, 1.05 g, 3 mmol) and acetone (30 mL) and the resulting mixture was stirred at RT for 5 min. An aq. solution of HCl (2 M, 4 mL) was added and stirring was continued at 60° C. for 4 h. The solvents were removed and the crude residue was purified by preparative HPLC on an XBridge C18 column (250×21.2 mm, 10 μm) eluting with 5% to 95% ACN in aq. NH₄HCO₃ (0.01 M) to provide the title compound C3-2 as a solid (520 mg).

LC-MS Method B: Rt=1.36 min; [M+H]⁺=339.

### Step 3: 1-((1-(2-(4-((1-(2,5-dimethoxy-4-(2-methyl-1-oxo-1,2-dihydro-2,7-naphthyridin-4-yl)phenethyl)piperidin-4-yl)oxy)piperidin-1-yl)ethyl)-2-oxo-1,2-dihydropyridin-3-yl)methyl)dihydropyrimidine-2,4(1H,3H)-dione (Compound C3)

To a 25 mL round bottom flask was added 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione (Intermediate 40, 43 mg, 0.1 mmol), 2-(2,5-dimethoxy-4-(2-methyl-1-oxo-1,2-dihydro-2,7-naphthyridin-4-yl)phenyl)acetaldehyde (C3-2, 53 mg, 0.15 mmol), a solution of ZnCl₂ (1 M) in THE (0.15 mL, 0.15 mmol), and DMSO (1 mL) and the RM was stirred at RT for 2 h. Solid NaBH₃CN (13 mg, 0.2 mmol) and MeOH (0.3 mL) were added and the RM was stirred at 30° C. for 16 h. The solvents were removed and the crude residue was purified by preparative HPLC on an XBridge C18 column (250×21.2 mm, 10 μm) eluting with 5% to 80% ACN in aq. NH₄HCO₃ (0.01 M) to provide the title compound Compound C3 as a solid (27 mg).

LC-MS Method C: Rt=1.67 min; [M/2+H]⁺=377.8.

¹H NMR (500 MHz, DMSO-d₆) δ 10.16 (s, 1H), 9.40 (s, 1H), 8.63 (d, J=5.6 Hz, 1H), 7.72 (s, 1H), 7.58 (dd, J=6.7, 1.6 Hz, 1H), 7.27 (d, J=5.5 Hz, 1H), 7.03 (s, 1H), 7.01 (d, J=5.7 Hz, 1H), 6.88 (s, 1H), 6.20 (t, J=6.8 Hz, 1H), 4.26 (s, 2H), 3.99 (t, J=6.3 Hz, 2H), 3.75 (s, 3H), 3.62 (s, 3H), 3.57 (s, 3H), 3.46-3.36 (m, 4H), 3.33-3.30 (m, 2H), 2.92-2.67 (m, 6H), 2.57 (t, J 6.8 Hz, 2H), 2.53-2.50 (m, 2H), 2.12 (t, J=9.4 Hz, 4H), 1.87-1.71 (m, 4H), 1.43-1.34 (m, 4H).

### Synthesis of Compound C4: (1-((1-(2-(4-((1-(2,6-dimethoxy-4-(2-methyl-1-oxo-1,2-dihydro-2,7-naphthyridin-4-yl)phenethyl)piperidin-4-yl)oxy)piperidin-1-yl)ethyl)-2-oxo-1,2-dihydropyridin-3-yl)methyl)dihydropyrimidine-2,4(1H,3H)-dione)

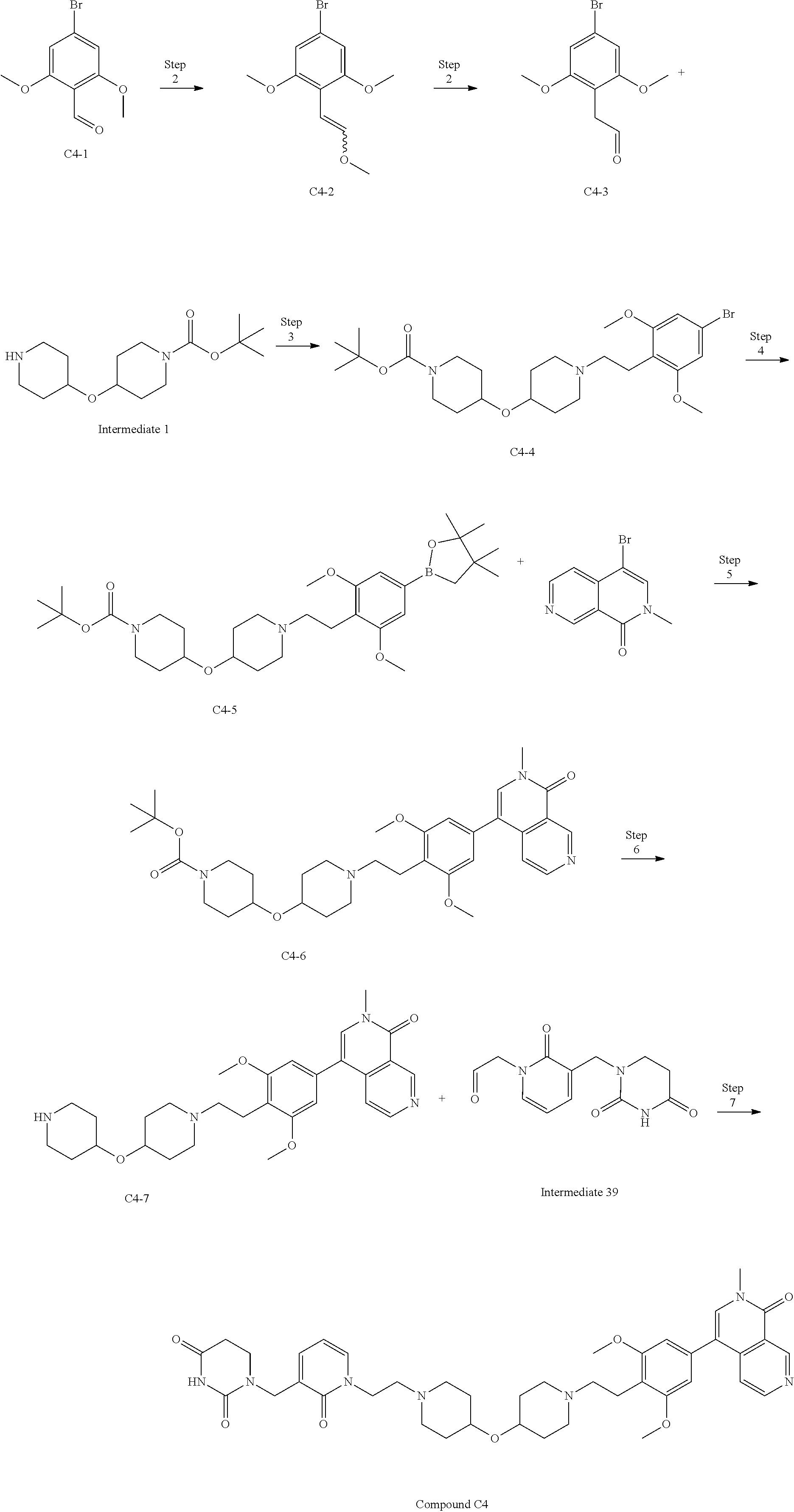

### Step 1: 5-bromo-1,3-dimethoxy-2-(2-methoxyvinyl)benzene (C4-2)

To a 250 mL round bottom flask was added (methoxymethyl)triphenylphosphonium chloride (21 g, 61.5 mmol), t-BuOK (9.2 g, 82 mmol), and THF (100 mL) and the resulting mixture was stirred at 0° C. for 30 min. 4-Bromo-2,6-dimethoxybenzaldehyde (C4-1, 5 g, 20.5 mmol) was added and stirring was continued at 0° C. for 1 h and then at 70° C. for 16 h. The RM was added into water (100 mL) and extracted with EtOAc (200 mL). The organic phase was washed with brine (100 mL), dried over Na₂SO₄, filtered, and concentrated. The crude residue was purified by chromatography on silica gel eluting with 0% to 100% EtOAc in PE to provide the title compound C4-2 as a solid (3 g).

LC-MS Method H: Rt=2.11 min; [M+H]⁺=273.

### Step 2: 2-(4-bromo-2,6-dimethoxyphenyl)acetaldehyde (C4-3)

To a 250 mL round bottom flask was added 5-bromo-1,3-dimethoxy-2-(2-methoxyvinyl)benzene (C4-2, 3 g, 11.03 mmol), acetone (40 mL), and an aq. solution of H₂SO₄ (2 M, 24 mL) and the resulting mixture was stirred at 65° C. for 3 h. The RM was added into water (50 mL) and extracted with EtOAc (150 mL). The organic phase was washed with brine (50 mL), dried over Na₂SO₄, filtered, and concentrated. The resulting yellow oil (3.3 g), containing the title compound C4-3, was directly used in the next step without further purification.

LC-MS Method H: Rt=2.05 min; [M+H]⁺=261.

### Step 3: tert-butyl 4-((1-(4-bromo-2,6-dimethoxyphenethyl)piperidin-4-yl)oxy)piperidine-1-carboxylate (C4-4)

To a 250 mL round bottom flask was added 2-(4-bromo-2,6-dimethoxyphenyl)acetaldehyde (C4-3, 3.3 g, 12.7 mmol), tert-butyl 4-(piperidin-4-yloxy)piperidine-1-carboxylate (Intermediate 1, 4.33 g, 15.2 mmol), a solution of ZnCl₂ (1 M) in THE (16.5 mL, 16.5 mmol), and DMSO (40 mL) and the resulting mixture was stirred at RT for 1 h. Solid NaBH₃CN (1.6 g, 25.4 mmol) was added and stirring was continued at RT for 16 h. The RM was added into water (50 mL) and extracted with EtOAc (150 mL). The organic phase was washed with brine (50 mL), dried over Na₂SO₄, filtered, and concentrated. The crude residue was purified by reversed phase chromatography on a Biotage Agela C18 column (120 g, spherical 20-35 μm, 100 Å) eluting with 5% to 95% ACN in aq. TFA (0.1%) to provide the title compound as a solid (2.3 g).

LC-MS Method A: Rt=1.39 min; [M+H]⁺=527.

### Step 4: tert-butyl 4-((1-(2,6-dimethoxy-4-(4,4,5,5-tetramethyl-1,3,2-dioxaborolan-2-yl)phenethyl)piperidin-4-yl)oxy)piperidine-1-carboxylate (C4-5)

To a 100 mL round bottom flask was added tert-butyl 4-((1-(4-bromo-2,6-dimethoxyphenethyl)piperidin-4-yl)oxy)piperidine-1-carboxylate (C4-4, 2.3 g, 4 mmol), 4,4,4′,4′,5,5,5′,5′-octamethyl-2,2′-bi(1,3,2-dioxaborolane) (BISPIN) (1.32 g, 5.2 mmol), K₂CO₃ (1.38 g, 10 mmol), 1,4-dioxane (20 mL) and PdCl₂(dppf) (146 mg, 0.2 mmol) and the resulting mixture was stirred under an atmosphere of N₂ at 100° C. for 16 h. The RM was added into water (50 mL) and extracted with EtOAc (150 mL). The organic phase was washed with brine (50 mL), dried over Na₂SO₄, filtered, and concentrated. The crude residue was purified by reversed phase chromatography on a Biotage Agela C18 column (120 g, spherical 20-35 μm, 100 Å) eluting with 5% to 95% ACN in aq. TFA (0.1%) to provide the title compound C4-5 as a solid (1.5 g).

LC-MS Method A: Rt=1.46 min; [M+H]⁺ 575.

### Step 5: tert-butyl 4-((1-(2,6-dimethoxy-4-(2-methyl-1-oxo-1,2-dihydro-2,7-naphthyridin-4-yl)phenethyl)piperidin-4-yl)oxy)piperidine-1-carboxylate (C4-6)

To a 250 mL round bottom flask was added tert-butyl 4-((1-(2,6-dimethoxy-4-(4,4,5,5-tetramethyl-1,3,2-dioxaborolan-2-yl)phenethyl)piperidin-4-yl)oxy)piperidine-1-carboxylate (C4-5, 100 mg, 0.42 mmol), 4-bromo-2-methyl-2,7-naphthyridin-1(2H)-one (Intermediate 5, 288 mg, 0.5 mmol), Na₂CO₃ (134 mg, 1.26 mmol), 1,4-dioxane (5 mL), water (1 mL) and PdCl₂(dppf) (30 mg, 0.04 mmol) and the resulting mixture was stirred under an atmosphere of N₂ at 100° C. for 4 h. Water (50 mL) was added, the RM was extracted with EtOAc (150 mL). The organic phase was washed with brine (50 mL), dried over Na₂SO₄, filtered, and concentrated. The crude residue was purified by reversed phase chromatography on a Biotage Agela C18 column (120 g, spherical 20-35 μm, 100 Å) eluting with 5% to 95% ACN in aq. TFA (0.1%) to provide the title compound C4-6 as a solid (120 mg).

LC-MS Method A: Rt=1.25 min; [M+H]⁺=607.

### Step 6: 4-(3,5-dimethoxy-4-(2-(4-(piperidin-4-yloxy)piperidin-1-yl)ethyl)phenyl)-2-methyl-2,7-naphthyridin-1(2H)-one (C4-7)

To a 25 mL round bottom flask was added tert-butyl 4-((1-(2,6-dimethoxy-4-(2-methyl-1-oxo-1,2-dihydro-2,7-naphthyridin-4-yl)phenethyl)piperidin-4-yl)oxy)piperidine-1-carboxylate (C4-6, 120 mg, 0.20 mmol) ), a solution of HCl (4 M) in 1,4-dioxane (2 mL), DCM (4 mL), and MeOH (1 mL) and the resulting mixture was stirred at RT for 3 h. The solvents were removed and the obtained solid (120 mg), containing the hydrochloride salt of the title compound C4-7, was directly used in the next step without further purification.

LC-MS Method H: Rt=0.99 min; [M+H]⁺=507.

### Step 7: 1-((1-(2-(4-((1-(2,6-dimethoxy-4-(2-methyl-1-oxo-1,2-dihydro-2,7-naphthyridin-4-yl)phenethyl)piperidin-4-yl)oxy)piperidin-1-yl)ethyl)-2-oxo-1,2-dihydropyridin-3-yl)methyl)dihydropyrimidine-2,4(1H,3H)-dione (Compound C4)

To a 250 mL round bottom flask was added 4-(3,5-dimethoxy-4-(2-(4-(piperidin-4-yloxy)piperidin-1-yl)ethyl)phenyl)-2-methyl-2,7-naphthyridin-1(2H)-one hydrochloride (C4-7, 120 mg, 0.24 mmol), 2-(3-((2,4-dioxotetrahydropyrimidin-1(2H)-yl)methyl)-2-oxopyridin-1(2H)-yl)acetaldehyde (Intermediate 39, 76 mg 0.29 mmol), a solution of ZnCl₂ (1 M) in THE (0.48 mL, 0.48 mmol), and DMSO (3 mL) and the resulting RM was stirred at RT for 1 h. Solid NaBH₃CN (30 mg, 0.48 mmol) was added and the RM was stirred at RT for 16 h. The solvents were removed and the crude residue was purified by preparative HPLC on an XBridge C18 column (250×21.2 mm, 10 μm) eluting with 5% to 95% ACN in aq. NH₄HCO₃ (0.01 M) to provide the title compound Compound C4 as a solid (11 mg).

LC-MS Method H: Rt=1.59 min; [M+H]⁺=754.

¹H NMR (500 MHz, DMSO-d₆) δ 10.17 (s, 1H), 9.43 (s, 1H), 8.71 (d, J=5.7 Hz, 1H), 7.85 (s, 1H), 7.63-7.48 (m, 2H), 7.27 (d, J=6.4 Hz, 1H), 6.71 (d, J=20.3 Hz, 2H), 6.20 (t, J=6.8 Hz, 1H), 4.26 (s, 2H), 3.99 (t, J=6.4 Hz, 2H), 3.80 (s, 6H), 3.59 (s, 3H), 3.43-3.37 (m, 4H), 2.80-2.69 (m, 6H), 2.65-2.51 (m, 4H), 2.36-2.29 (m, 2H), 2.16-2.04 (m, 4H), 1.77 (t, J=13.2 Hz, 4H), 1.44-1.32 (m, 4H).

### Compound D1: 1-((1-(2-(4-((1-(4-(2-butyl-1-oxo-1,2-dihydro-2,7-naphthyridin-4-yl)-2,5-dimethoxybenzyl)piperidin-4-yl)oxy)piperidin-1-yl)ethyl)-2-oxo-1,2-dihydropyridin-3-yl)methyl)dihydropyrimidine-2,4(1H,3H)-dione

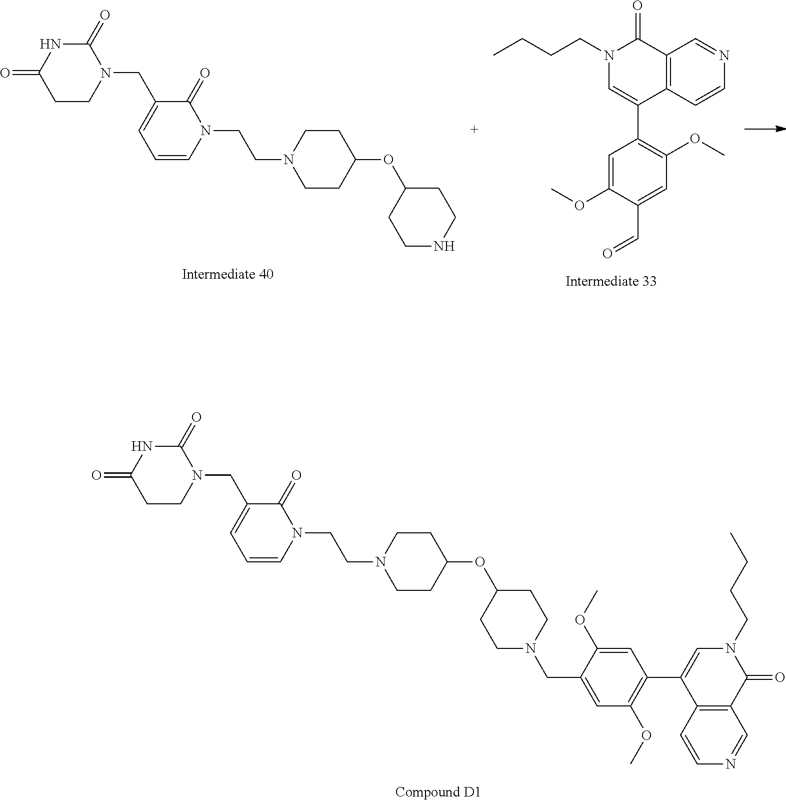 [SMILES: O=C1CCN(Cc2cccn(CCN3CCC(OC4CCNCC4)CC3)c2=O)C(=O)N1 ; CCCCn1cc(-c2cc(OC)c(C=O)cc2OC)c2ccncc2c1=O ; CCCCn1cc(-c2cc(OC)c(CN3CCC(OC4CCN(CCn5cccc(CN6CCC(=O)NC6=O)c5=O)CC4)CC3)cc2OC)c2ccncc2c1=O]

To a solution of 4-(2-butyl-1-oxo-1,2-dihydro-2,7-naphthyridin-4-yl)-2,5-dimethoxybenzaldehyde (Intermediate 33, 100 mg, 0.272 mmol) and 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione hydrochloride (Intermediate 40, 127.6 mg, 0.272 mmol) in DMSO (1 ml) was added DIEA (38.80 mg, 0.30 mmol) and a solution of ZnCl₂ (1 M) in THE (0.545 ml, 0.545 mmol) and the RM was stirred at 25° C. for 2 h. Solid NaBH₃CN (51.45 mg, 0.818 mmol) was added and the RM was stirred at 25° C. for 12 h. The mixture was added into MeOH (10 ml) and filtered, the filtrate was concentrated and an aq. sat. solution of NH₄Cl (20 mL) was added. The aq. phase was extracted with EtOAc (2×20 ml), the combined organic phases were washed with brine (10 ml), dried over Na₂SO₄ and concentrated. The residue was purified by reverse phase chromatography on a Phenomenex Synergi C18 column (25×150 mm, 10 μm), eluting with ACN (from 10% to 40%) in an aq. solution of HCOOH (0.225%), yielding the title compound 1-((1-(2-(4-((1-(4-(2-butyl-1-oxo-1,2-dihydro-2,7-naphthyridin-4-yl)-2,5-dimethoxybenzyl)piperidin-4-yl)oxy)piperidin-1-yl)ethyl)-2-oxo-1,2-dihydropyridin-3-yl)methyl)dihydropyrimidine-2,4(1H,3H)-dione, Compound D1, as a solid (26.91 mg).

Method LCMS WX4: Rt=0.953 min; [M+H]⁺=782.6.

¹H NMR (400 MHz, DMSO-d₆) δ[ppm] 10.15 (s, 1H), 9.41 (s, 1H), 8.65 (d, J=5.6 Hz, 1H), 8.19 (s, 2H), 7.73 (s, 1H), 7.58 (dd, J=1.8, 6.8 Hz, 1H), 7.32-7.25 (m, 1H), 7.15 (s, 1H), 7.06 (d, J=5.6 Hz, 1H), 6.92 (s, 1H), 6.21 (t, J=6.8 Hz, 1H), 4.27 (s, 2H), 4.00 (t, J=6.8 Hz, 3H), 3.75 (s, 3H), 3.64 (s, 3H), 3.52 (s, 3H), 2.75 (d, J=3.6 Hz, 3H), 2.69-2.67 (m, 1H), 2.35-2.33 (m, 1H), 2.15 (dt, J=2.4, 6.8 Hz, 6H), 1.85-1.71 (m, 7H), 1.52-1.34 (m, 7H), 0.93 (t, J=7.2 Hz, 4H).

### Compound D2: 1-((1-(2-(4-((1-(4-(2-butyl-1-oxo-1,2-dihydro-2,7-naphthyridin-4-yl)-2,6-dimethoxybenzyl)piperidin-4-yl)oxy)piperidin-1-yl)ethyl)-2-oxo-1,2-dihydropyridin-3-yl)methyl)dihydropyrimidine-2,4(1H,3H)-dione

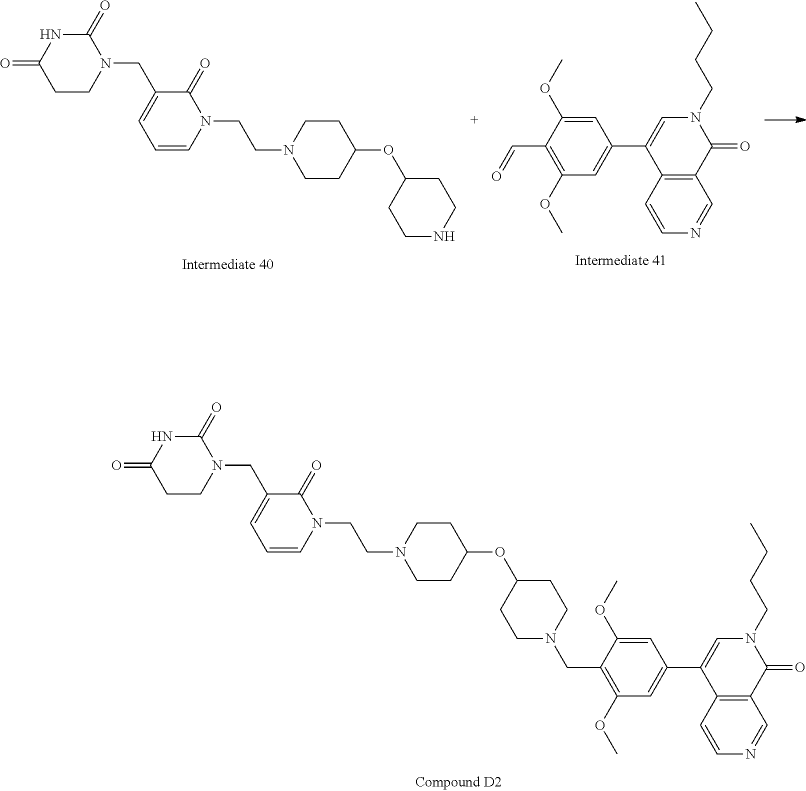

### Step 1: 1-((1-(2-(4-((1-(4-(2-butyl-1-oxo-1,2-dihydro-2,7-naphthyridin-4-yl)-2,6-dimethoxybenzyl)piperidin-4-yl)oxy)piperidin-1-yl)ethyl)-2-oxo-1,2-dihydropyridin-3-yl)methyl)dihydropyrimidine-2,4(1H,3H)-dione (Compound D2)

To a 25 ml round bottom flask was added 4-(2-butyl-1-oxo-1,2-dihydro-2,7-naphthyridin-4-yl)-2,6-dimethoxybenzaldehyde (Intermediate 41, 100 mg, 0.272 mmol), 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione hydrochloride (Intermediate 40, 127.6 mg, 0.272 mmol), DMSO (1 mL), DIEA (38.80 mg, 0.053 mL, 0.30 mmol) and a solution of ZnCl₂ (1 M) in THE (0.54 mL, 0.54 mmol) and the RM was stirred at 25° C. for 2 h. Solid NaBH₃CN (51.45 mg, 0.818 mmol) was added and stirring was continued for 12 h. The mixture was concentrated and the residue was purified by preparative HPLC on a Phenomenex Gemini-NX C18 column (75×30 mm×3 μm) eluting with ACN (from 18% to 28%) in an aq. solution of TFA (0.1%). Fractions containing the title compound were combined, concentrated and the residue was purified by preparative HPLC on a Waters XBridge column (150×25 mm, 5 μm) eluting with ACN (from 12% to 42%) in an aq solution of NaHCO₃(10 mM), yielding the title compound 1-((1-(2-(4-((1-(4-(2-butyl-1-oxo-1,2-dihydro-2,7-naphthyridin-4-yl)-2,6-dimethoxybenzyl)piperidin-4-yl)oxy)piperidin-1-yl)ethyl)-2-oxo-1,2-dihydropyridin-3-yl)methyl)dihydropyrimidine-2,4(1H,3H)-dione, Compound D2, as a solid (15 mg).

Method LCMS WX2: Rt=0.709 min; [M+H]⁺=782.4.

¹H NMR (400 MHz, chloroform-d) δ [ppm] 9.67-9.59 (m, 1H), 8.63 (d, J=5.6 Hz, 1H), 7.43-7.35 (m, 2H), 7.23 (dd, J=2.0, 6.8 Hz, 1H), 7.20 (s, 1H), 6.48 (s, 2H), 6.08 (t, J=6.8 Hz, 1H), 4.37 (s, 2H), 4.01-3.94 (m, 4H), 3.77 (s, 7H), 3.70-3.49 (m, 5H), 3.42-3.27 (m, 2H), 2.95-2.81 (m, 2H), 2.78-2.67 (m, 2H), 2.64-2.50 (m, 5H), 2.13 (t, J=9.6 Hz, 3H), 1.79-1.70 (m, 6H), 1.42-1.32 (m, 3H), 1.21-1.15 (m, 2H), 0.92 (t, J=7.3 Hz, 3H).

### Compound D3: 1-((1-(2-(4-((1-(4-(2-butyl-1-oxo-1,2-dihydro-2,7-naphthyridin-4-yl)-2,5-dimethoxyphenethyl)piperidin-4-yl)oxy)piperidin-1-yl)ethyl)-2-oxo-1,2-dihydropyridin-3-yl)methyl)dihydropyrimidine-2,4(1H,3H)-dione

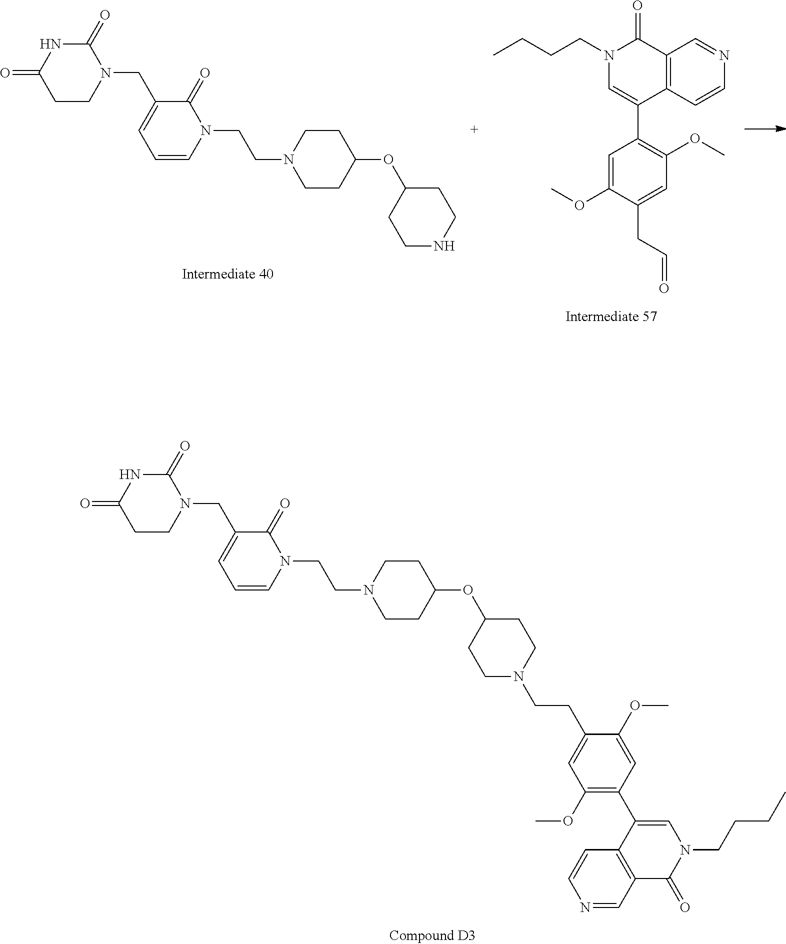 [SMILES: O=C1CCN(Cc2cccn(CCN3CCC(OC4CCNCC4)CC3)c2=O)C(=O)N1 ; CCCCn1cc(-c2cc(OC)c(CC=O)cc2OC)c2ccncc2c1=O ; CCCCn1cc(-c2cc(OC)c(CCN3CCC(OC4CCN(CCn5cccc(CN6CCC(=O)NC6=O)c5=O)CC4)CC3)cc2OC)c2ccncc2c1=O]

To a solution of 2-(4-(2-butyl-1-oxo-1,2-dihydro-2,7-naphthyridin-4-yl)-2,5-dimethoxyphenyl)acetaldehyde (Intermediate 57, 200 mg, 0.526 mmol) and 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione hydrochloride (Intermediate 40, 246 mg, 0.526 mmol) in DMSO (5 ml) was added DIEA (74 mg, 0.579 mmol) and a solution of ZnCl₂ (1 M) in THE (1.1 ml, 1.1 mmol) and the RM was stirred at 25° C. for 2 h. Solid NaBH₃CN (99 mg, 1.578 mmol) was added and the RM was stirred at 25° C. for 14 h. The mixture was added into water (20 ml), the aq. phase was extracted with DCM (3×30 ml), the combined organic phases were concentrated and the residue was purified by preparative HPLC on an XBridge C18 column (250×21.2 mm, 10 μm), eluting with ACN (from 10% to 50%) in an aq. solution of NH₄HCO₃ (0.01 M), yielding the title compound 1-((1-(2-(4-((1-(4-(2-butyl-1-oxo-1,2-dihydro-2,7-naphthyridin-4-yl)-2,5-dimethoxyphenethyl)piperidin-4-yl)oxy)piperidin-1-yl)ethyl)-2-oxo-1,2-dihydropyridin-3-yl)methyl)dihydropyrimidine-2,4(1H,3H)-dione, Compound D3, as a solid (22.01 mg).

Method LCMS WX4: Rt=0.950 min; [M+H]⁺=796.8.

¹H NMR (400 MHz, DMSO-d₆) δ[ppm] 10.14 (s, 1H), 9.44 (s, 1H), 8.70 (d, J=5.6 Hz, 1H), 7.80 (s, 1H), 7.56 (t, J=6.4 Hz, 2H), 7.27 (d, J=6.0 Hz, 1H), 6.69 (s, 2H), 6.20 (t, J=6.4 Hz, 1H), 4.27 (s, 2H), 4.01 (td, J=6.4, 18.8 Hz, 4H), 3.81 (s, 6H), 3.41 (t, J=6.6 Hz, 3H), 3.29-3.26 (m, 3H), 3.17 (d, J=5.0 Hz, 1H), 2.88-2.65 (m, 6H), 2.45-2.30 (m, 2H), 2.22-2.00 (m, 5H), 1.85-1.66 (m, 6H), 1.52-1.26 (m, 6H), 0.99-0.87 (m, 3H).

### Compound D4: 1-((1-(2-(4-((1-(4-(2-butyl-1-oxo-1,2-dihydro-2,7-naphthyridin-4-yl)-2,6-dimethoxyphenethyl)piperidin-4-yl)oxy)piperidin-1-yl)ethyl)-2-oxo-1,2-dihydropyridin-3-yl)methyl)dihydropyrimidine-2,4(1H,3H)-dione

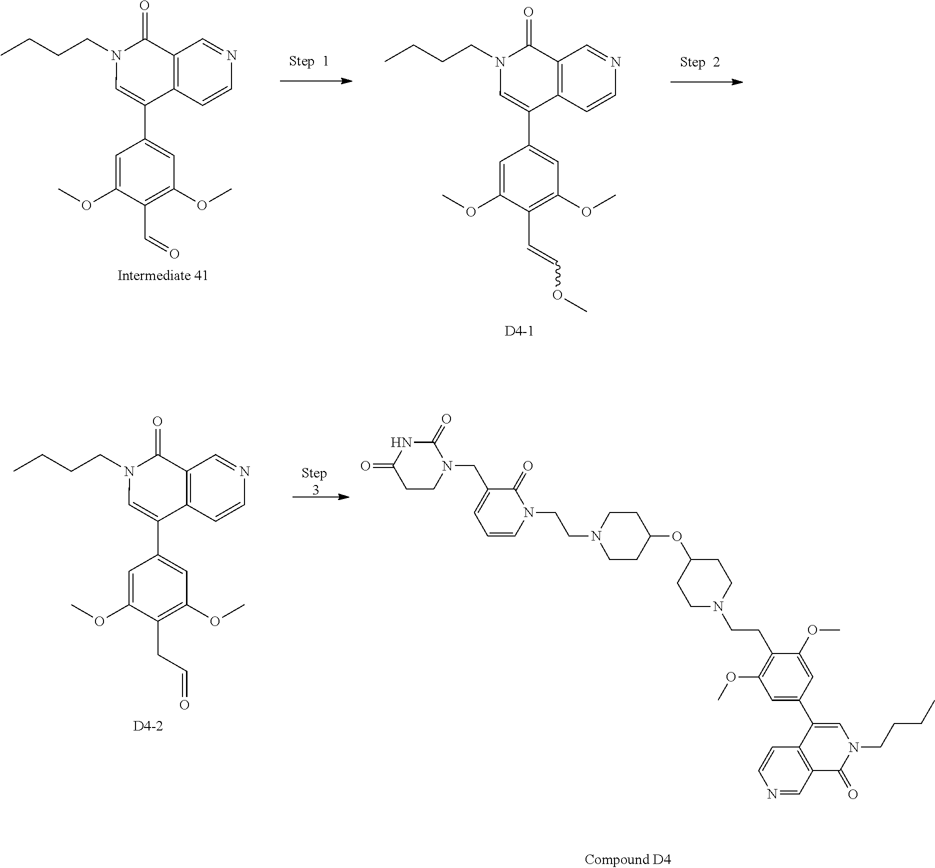 [SMILES: CCCCn1cc(-c2cc(OC)c(C=O)c(OC)c2)c2ccncc2c1=O ; CCCCn1cc(-c2cc(OC)c(C=COC)c(OC)c2)c2ccncc2c1=O ; "D4-1" ; CCCCn1cc(-c2cc(OC)c(CC=O)c(OC)c2)c2ccncc2c1=O ; "D4-2" ; CCCCn1cc(-c2cc(OC)c(CCN3CCC(OC4CCN(CCn5cccc(CN6CCC(=O)NC6=O)c5=O)CC4)CC3)c(OC)c2)c2ccncc2c1=O]

### Step 1: 2-butyl-4-(3,5-dimethoxy-4-(2-methoxyvinyl)phenyl)-2,7-naphthyridin-1(2H)-one (D4-1)

To a solution of chloro(methoxymethyl)triphenylphosphorane (375 mg, 1.09 mmol) in THF (4 ml) at 0° C. was added a solution of t-BuOK (146 mg, 1.31 mmol) in TH (4 ml). The RM was stirred 0.5 h at 0° C. and a solution of 4-(2-butyl-1-oxo-1,2-dihydro-2,7-naphthyridin-4-yl)-2,6-dimethoxybenzaldehyde (Intermediate 41, 400 mg, 1.09 mmol) in THE (4 ml) was added at 0° C. The cooling bath was removed and the RM was stirred at 70° C. for 15.5 h. The mixture was added into water (20 ml), the aq. phase was extracted with DCM (3×20 ml), the combined organic phases were washed with brine (20 ml), dried over Na₂SO₄ and concentrated. The residue was purified by chromatography on silica gel on a SepaFlash® Silica Flash column (80 g), eluting with EtOAc (from 0% to 100%) in PE, yielding the title compound 2-butyl-4-(3,5-dimethoxy-4-(2-methoxyvinyl)phenyl)-2,7-naphthyridin-1(2H)-one, D4-1, as an oil (500 mg).

Method LCMS WX2: Rt=0.916 min; [M+H]⁺=395.2.

### Step 2: 2-(4-(2-butyl-1-oxo-1,2-dihydro-2,7-naphthyridin-4-yl)-2,6-dimethoxyphenyl)acetaldehyde (D4-2)

To a solution of 2-butyl-4-(3,5-dimethoxy-4-(2-methoxyvinyl)phenyl)-2,7-naphthyridin-1(2H)-one (D4-1, 500 mg, 0.76 mmol) in THF (3.5 ml) was added an aq. solution of HCl (2 M, 0.5 ml, 1 mmol) and the RM was stirred at 25° C. for 2 h. The mixture was added into water (10 ml), the aq. phase was extracted with EtOAc (3×20 ml), the combined organic phases were concentrated, yielding the title compound 2-(4-(2-butyl-1-oxo-1,2-dihydro-2,7-naphthyridin-4-yl)-2,6-dimethoxyphenyl)acetaldehyde, D4-2, as a solid HCl salt (300 mg), which was used for the next step without further purification.

Method LCMS WX4: Rt=0.835 min; [M+H]⁺=381.2.

### Step 3: 1-((1-(2-(4-((1-(4-(2-butyl-1-oxo-1,2-dihydro-2,7-naphthyridin-4-yl)-2,6-dimethoxyphenethyl)piperidin-4-yl)oxy)piperidin-1-yl)ethyl)-2-oxo-1,2-dihydropyridin-3-yl)methyl)dihydropyrimidine-2,4(1H,3H)-dione (Compound D4)

To a 25 ml round bottom flask were added 2-(4-(2-butyl-1-oxo-1,2-dihydro-2,7-naphthyridin-4-yl)-2,6-dimethoxyphenyl)acetaldehyde HCl salt (D4-2, 150 mg, 0.394 mmol), 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione hydrochloride (Intermediate 40, 170 mg, 0.394 mmol), DMSO (5 ml), DIEA (56 mg, 0.433 mmol) and a solution of ZnCl₂ (1 M) in THE (0.8 ml, 0.8 mmol) and the RM was stirred at 25° C. for 2 h. Solid NaBH₃CN (74 mg, 1.182 mmol) was added and the RM was stirred at 25° C. for 14 h. The mixture was added into water (20 ml), the aq. phase was extracted with DCM (3×30 ml), the combined organic phases were concentrated and the residue was purified by preparative HPLC on an XBridge C18 column (250×21.2 mm, 10 μm) eluting with ACN (from 10% to 50%) in an aq. solution of NH₄HCO₃ (0.01 M), yielding the title compound 1-((1-(2-(4-((1-(4-(2-butyl-1-oxo-1,2-dihydro-2,7-naphthyridin-4-yl)-2,6-dimethoxyphenethyl)piperidin-4-yl)oxy)piperidin-1-yl)ethyl)-2-oxo-1,2-dihydropyridin-3-yl)methyl)dihydropyrimidine-2,4(1H,3H)-dione, Compound D4, as a solid (30.04 mg).

Method LCMS WX2: Rt=0.731 min; [M+H]⁺=796.8.

¹H NMR (400 MHz, DMSO-d₆) δ[ppm] 10.14 (s, 1H), 9.44 (s, 1H), 8.70 (d, J=5.6 Hz, 1H), 7.80 (s, 1H), 7.56 (t, J=6.6 Hz, 2H), 7.27 (d, J=6.0 Hz, 1H), 6.69 (s, 2H), 6.20 (t, J=6.6 Hz, 1H), 4.27 (s, 2H), 4.01 (td, J=6.6, 18.6 Hz, 4H), 3.81 (s, 6H), 3.41 (t, J=6.6 Hz, 2H), 3.29-3.26 (m, 3H), 3.17 (d, J=5.0 Hz, 1H), 2.88-2.65 (m, 6H), 2.45-2.30 (m, 2H), 2.22-2.00 (m, 6H), 1.85-1.66 (m, 6H), 1.52-1.26 (m, 6H), 0.99-0.87 (m, 3H).

### Compound E1: 1-((1-(2-(4-((1-(2,5-difluoro-4-(2-methyl-1-oxo-1,2-dihydro-2,7-naphthyridin-4-yl)benzyl)piperidin-4-yl)oxy)piperidin-1-yl)ethyl)-2-oxo-1,2-dihydropyridin-3-yl)methyl)dihydropyrimidine-2,4(1H,3H)-dione

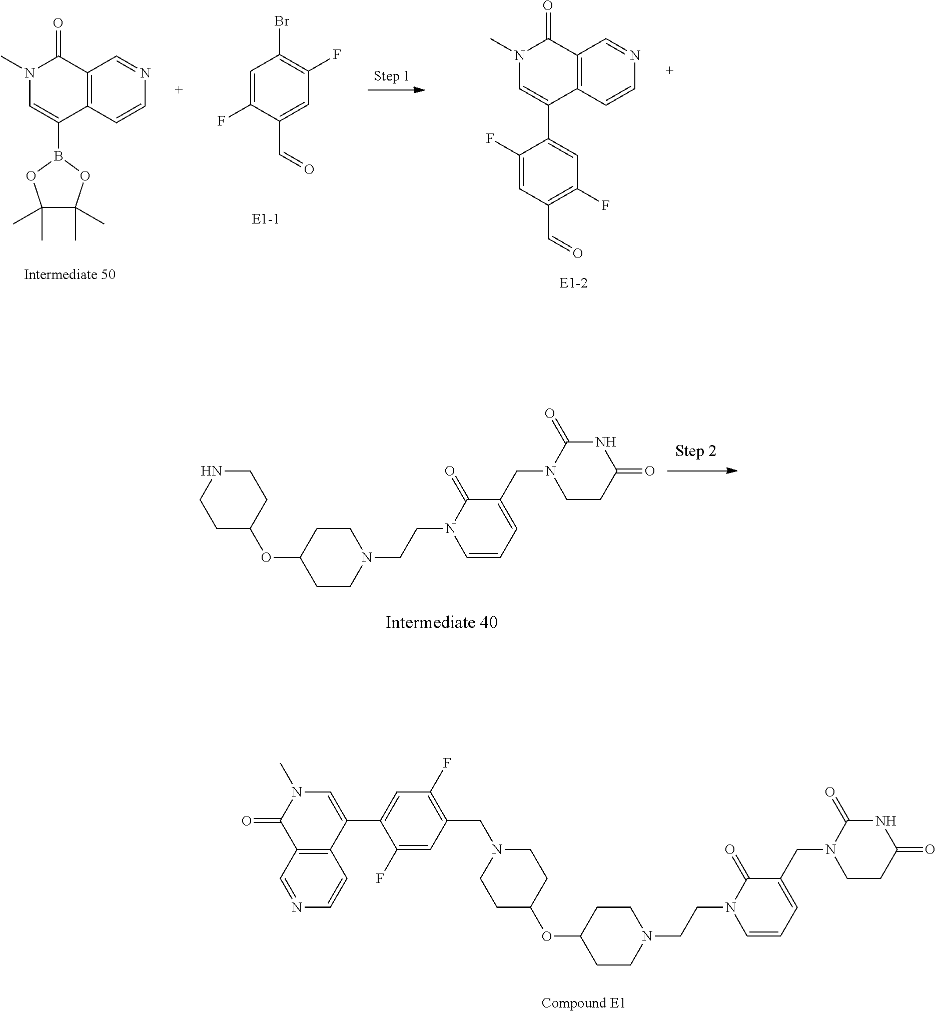 [SMILES: Cn1cc(B2OC(C)(C)C(C)(C)O2)c2ccncc2c1=O ; O=Cc1cc(F)c(Br)cc1F ; Cn1cc(-c2cc(F)c(C=O)cc2F)c2ccncc2c1=O ; O=C1CCN(Cc2cccn(CCN3CCC(OC4CCNCC4)CC3)c2=O)C(=O)N1 ; Cn1cc(-c2cc(F)c(CN3CCC(OC4CCN(CCn5cccc(CN6CCC(=O)NC6=O)c5=O)CC4)CC3)cc2F)c2ccncc2c1=O]

### Step 1: 2,5-difluoro-4-(2-methyl-1-oxo-1,2-dihydro-2,7-naphthyridin-4-yl)benzaldehyde (E1-2)

To a mixture of 2-methyl-4-(4,4,5,5-tetramethyl-1,3,2-dioxaborolan-2-yl)-2,7-naphthyridin-1(2H)-one (Intermediate 50, 250 mg, 0.577 mmol), 4-bromo-2,5-difluorobenzaldehyde (E1-1, 153 mg, 0.692 mmol) and Na₂CO₃ (183 mg, 1.730 mmol) in a mixture of 1,4-dioxane (4 ml) and water (1 ml) under an argon atmosphere was added PdCl₂(dppf) (21 mg, 0.029 mmol) and the RM was heated at 100° C. for 2 h. The mixture was cooled to RT and filtered over CELITE®. The filtrate was partitioned between EtOAc and water and the aq. phase was extracted with EtOAc. The organic phase was washed with brine, dried over MgSO₄ and concentrated. The residue was triturated in MTBE. The mixture was filtered and the solids were dried, yielding the title compound 2,5-difluoro-4-(2-methyl-1-oxo-1,2-dihydro-2,7-naphthyridin-4-yl)benzaldehyde, E1-2, as a solid (206 mg), which was directly used for the next step without further purification.

Method LCMS_PL1: Rt=0.74 min; [M+H]⁺=301.

### Step 2: 1-((1-(2-(4-((1-(2,5-difluoro-4-(2-methyl-1-oxo-1,2-dihydro-2,7-naphthyridin-4-yl)benzyl)piperidin-4-yl)oxy)piperidin-1-yl)ethyl)-2-oxo-1,2-dihydropyridin-3-yl)methyl)dihydropyrimidine-2,4(1H,3H)-dione (Compound E1)

A mixture of NaOAc (27 mg, 0.317 mmol) and 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione TFA salt (Intermediate 40, 73 mg, 0.111 mmol) was in DCM (2 ml) was treated with ultrasound and stirred at RT for 2 min. DMF (1 ml) was added and the RM was stirred at RT for 2 min. HOAc (0.020 ml, 0.343 mmol) was added and the RM was stirred at RT for 10 min. 2,5-Difluoro-4-(2-methyl-1-oxo-1,2-dihydro-2,7-naphthyridin-4-yl)benzaldehyde (E1-2, 46 mg, 0.146 mmol) was added and the RM was stirred at RT for 1 h. Solid NaBH(OAc)₃ (46 mg, 0.217 mmol) was added and the RM was stirred at RT for three days. The mixture was concentrated and the residue was purified by reverse phase chromatography on a REDISEP® C18 column (15.5 g) eluting with ACN (1% to 100%) in an aq. solution of TFA (0.1%), followed by a purification using SFC on a Reprospher PEI column (100×50 mm, 100 Å, 3 m) eluting with MeOH (from 44% to 52%) in supercritical CO₂. Fractions containing the title compound were combined, concentrated and the residue was purified by reverse phase chromatography on a REDISEP® C18 column (15.5 g) eluting with ACN (1% to 100%) in an aq. solution of NH₄HCO₃ (0.1%), yielding the title compound 1-((1-(2-(4-((1-(2,5-difluoro-4-(2-methyl-1-oxo-1,2-dihydro-2,7-naphthyridin-4-yl)benzyl)piperidin-4-yl)oxy)piperidin-1-yl)ethyl)-2-oxo-1,2-dihydropyridin-3-yl)methyl)dihydropyrimidine-2,4(1H,3H)-dione, Compound E1, as a solid (4 mg).

Method LCMS_MLG9: Rt=0.42 min; [M+H]⁺=716.

¹H NMR (600 MHz, DMSO-d₆) δ[ppm] 10.15 (s, 1H), 9.44 (s, 1H), 8.72 (d, J=5.6 Hz, 1H), 7.94 (s, 1H), 7.59 (m, 1H), 7.33 (m, 3H), 7.19 (m, 1H), 6.21 (m, 1H), 4.27 (s, 2H), 3.99 (m, 1H), 3.58 (m, 4H), 3.42 (m, 6H), 2.73 (m, 4H), 2.58 (m, 4H), 2.27-2.01 (m, 4H), 1.77 (m, 4H), 1.43 (m, 4H).

### Compound E2: 1-((1-(2-((3R,4R)-4-((1-(2,5-dimethoxy-4-(2-methyl-1-oxo-1,2-dihydro-2,7-naphthyridin-4-yl)benzyl)piperidin-4-yl)oxy)-3-fluoropiperidin-1-yl)ethyl)-2-oxo-1,2-dihydropyridin-3-yl)methyl)dihydropyrimidine-2,4(1H,3H)-dione

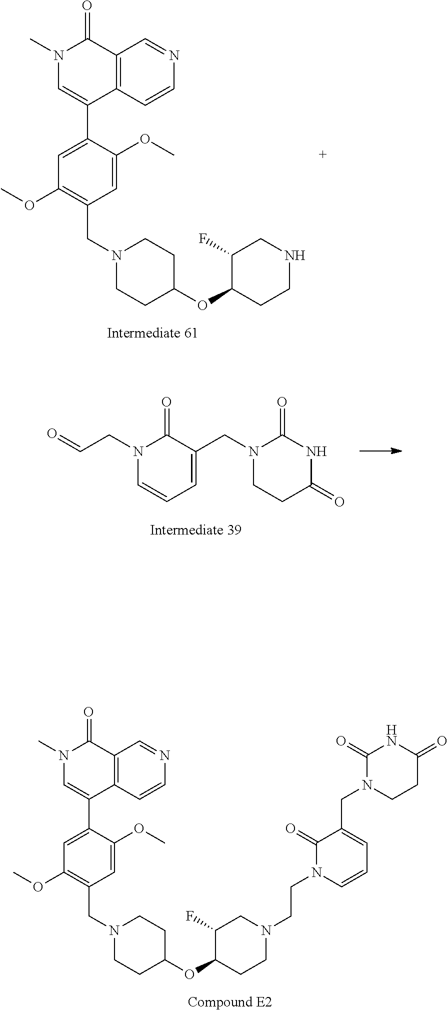 [SMILES: COc1cc(-c2cn(C)c(=O)c3cnccc23)c(OC)cc1CN1CCC(O[C@@H]2CCNC[C@H]2F)CC1 ; O=CCn1cccc(CN2CCC(=O)NC2=O)c1=O ; COc1cc(-c2cn(C)c(=O)c3cnccc23)c(OC)cc1CN1CCC(O[C@@H]2CCN(CCn3cccc(CN4CCC(=O)NC4=O)c3=O)C[C@H]2F)CC1]

To a solution of 2-(3-((2,4-dioxotetrahydropyrimidin-1(2H)-yl)methyl)-2-oxopyridin-1(2H)-yl)acetaldehyde (Intermediate 39, 100 mg, 0.190 mmol) in MeOH (2 ml) under an argon atmosphere were added 4-(4-((4-(((3R,4R)-3-fluoropiperidin-4-yl)oxy)piperidin-1-yl)methyl)-2,5-dimethoxyphenyl)-2-methyl-2,7-naphthyridin-1(2H)-one TFA salt (Intermediate 61, 100 mg, 0.135 mmol), DCM (1.5 ml), TEA (94 μl, 0.677 mmol) and a solution of ZnCl₂ (0.5 M) in THE (325 μl, 0.162 mmol) and the RM was stirred at RT for 3 h. A solution of NaBH₃CN (1 M) in THE (176 μl, 0.176 mmol) was added and the RM was stirred at RT for 20.5 h. The mixture was quenched with water, ACN and a few drops of TFA, adsorbed on ISOLUTE® HM-N and purified by reverse phase chromatography on a REDISEP® Gold HP C18 column (15.5 g) eluting with ACN (from 4.8% to 100%) in an aq. solution of TFA (0.1%). Fractions containing the title compound were filtered over PL-HCO₃ MP SPE cartridges and the combined filtrates were concentrated. The residue was taken up in MeOH and purified by preparative HPLC on a Waters™ X-Bridge™ C18 OBD™ column (100×30 mm, 5 μm) eluting with ACN (from 15% to 85%) in an aq. solution of NH₄OH (7.3 mM), yielding the title compound 1-((1-(2-((3R,4R)-4-((1-(2,5-dimethoxy-4-(2-methyl-1-oxo-1,2-dihydro-2,7-naphthyridin-4-yl)benzyl)piperidin-4-yl)oxy)-3-fluoropiperidin-1-yl)ethyl)-2-oxo-1,2-dihydropyridin-3-yl)methyl)dihydropyrimidine-2,4(1H,3H)-dione, Compound E2, as a solid (16 mg).

Method LCMS_MLG3: Rt=0.82 min; [M+H]⁺=758.

¹H NMR (600 MHz, DMSO) δ [ppm] 10.15 (s, 1H), 9.40 (d, J=0.9 Hz, 1H), 8.64 (d, J=5.6 Hz, 1H), 7.74 (s, 1H), 7.59 (dd, J=6.8, 2.0 Hz, 1H), 7.32-7.24 (m, 1H), 7.14 (s, 1H), 7.03 (dd, J=5.6, 0.9 Hz, 1H), 6.91 (s, 1H), 6.20 (t, J=6.8 Hz, 1H), 4.36-4.22 (m, 3H), 4.04-3.96 (m, 2H), 3.74 (s, 3H), 3.63 (s, 3H), 3.57 (s, 3H), 3.54-3.48 (m, 3H), 3.48-3.43 (m, 1H), 3.41 (t, J=6.8 Hz, 2H), 3.12-3.05 (m, 1H), 2.80-2.68 (m, 3H), 2.65-2.58 (m, 2H), 2.56 (t, J=6.8 Hz, 2H), 2.23-2.07 (m, 4H), 1.93-1.79 (m, 3H), 1.55-1.43 (m, 2H), 1.38-1.28 (m, 1H).

### Compound E3: 1-(5-(4-(((cis)-4-(4-(2-butyl-1-oxo-1,2-dihydro-2,7-naphthyridin-4 yl)phenoxy)cyclohexyl)oxy)piperidine-1-carbonyl)-2-methoxyphenyl)dihydropyrimidine-2,4(1H,3H)-dione

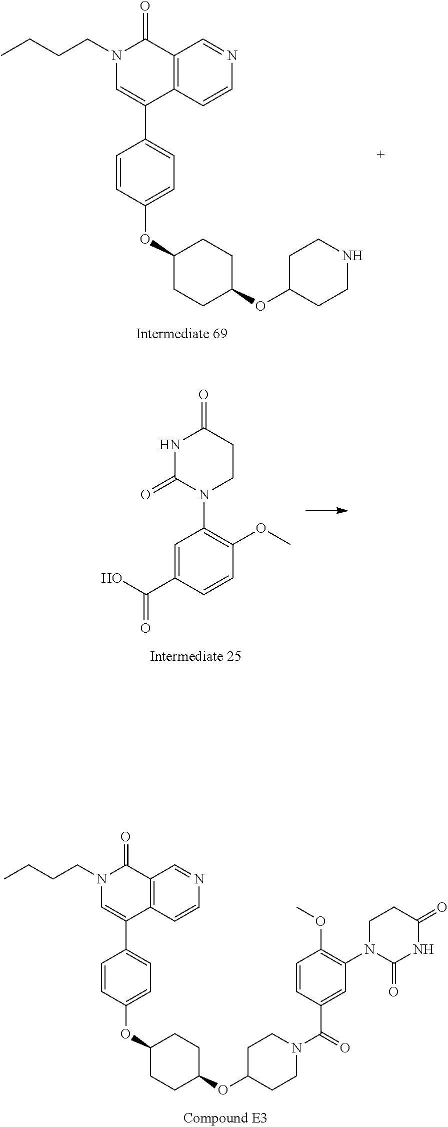 [SMILES: CCCCn1cc(-c2ccc(O[C@H]3CC[C@@H](OC4CCNCC4)CC3)cc2)c2ccncc2c1=O ; COc1ccc(C(=O)O)cc1N1CCC(=O)NC1=O ; CCCCn1cc(-c2ccc(O[C@H]3CC[C@@H](OC4CCN(C(=O)c5ccc(OC)c(N6CCC(=O)NC6=O)c5)CC4)CC3)cc2)c2ccncc2c1=O]

To a mixture of 3-(2,4-dioxotetrahydropyrimidin-1(2H)-yl)-4-methoxybenzoic acid (Intermediate 25, 34 mg, 0.129 mmol) in DMF (0.5 ml) were added DIEA (0.050 ml, 0.286 mmol) and HATU (54 mg, 0.142 mmol), and the RM was stirred at RT for 30 min. A solution of 2-butyl-4-(4-(((cis)-4-(piperidin-4-yloxy)cyclohexyl)oxy)phenyl)-2,7-naphthyridin-1(2H)-one (Intermediate 69, 73 mg, 0.118 mmol) and DIEA (0.050 ml, 0.286 mmol) in DMF (0.5 ml) was added and the RM was stirred at RT for 2 h. The mixture was purified by reverse phase chromatography on a REDISEP® Gold HP C18 column (15.5 g) eluting with ACN (from 1% to 100%) in an aq. solution of NH₄HCO₃ (0.1%), yielding the title compound 1-(5-(4-(((cis)-4-(4-(2-butyl-1-oxo-1,2-dihydro-2,7-naphthyridin-4 yl)phenoxy)cyclohexyl)oxy)piperidine-1-carbonyl)-2-methoxyphenyl)dihydropyrimidine-2,4(1H,3H)-dione, Compound E3, as a solid (22 mg).

Method LCMS_MLG4: Rt=4.80 min; [M+H]⁺=722.

¹H NMR (600 MHz, DMSO-d₆) δ[ppm] 10.33 (s, 1H), 9.43 (d, J=0.8 Hz, 1H), 8.71 (d, J=5.6 Hz, 1H), 7.74 (s, 1H), 7.43-7.34 (m, 5H), 7.16 (d, J=8.6 Hz, 1H), 7.11-7.07 (m, 2H), 4.51 (m, 1H), 4.03 (t, J=7.3 Hz, 2H), 3.84 (s, 3H), 3.74-3.57 (m, 5H), 3.27-3.21 (m, 2H), 2.68 (m, 2H), 1.83 (m, 4H), 1.75-1.61 (m, 9H), 1.45 (m, 2H), 1.33 (m, 2H), 0.92 (t, J=7.4 Hz, 3H).

### Compound E5: 1-(3-(4-(((3R,4R)-1-(4-(4,5-dimethyl-6-oxo-1-propyl-1,6-dihydropyridin-3-yl)benzyl)-3-fluoropiperidin-4-yl)oxy)piperidine-1-carbonyl)phenyl)dihydropyrimidine-2,4(1H,3H)-dione

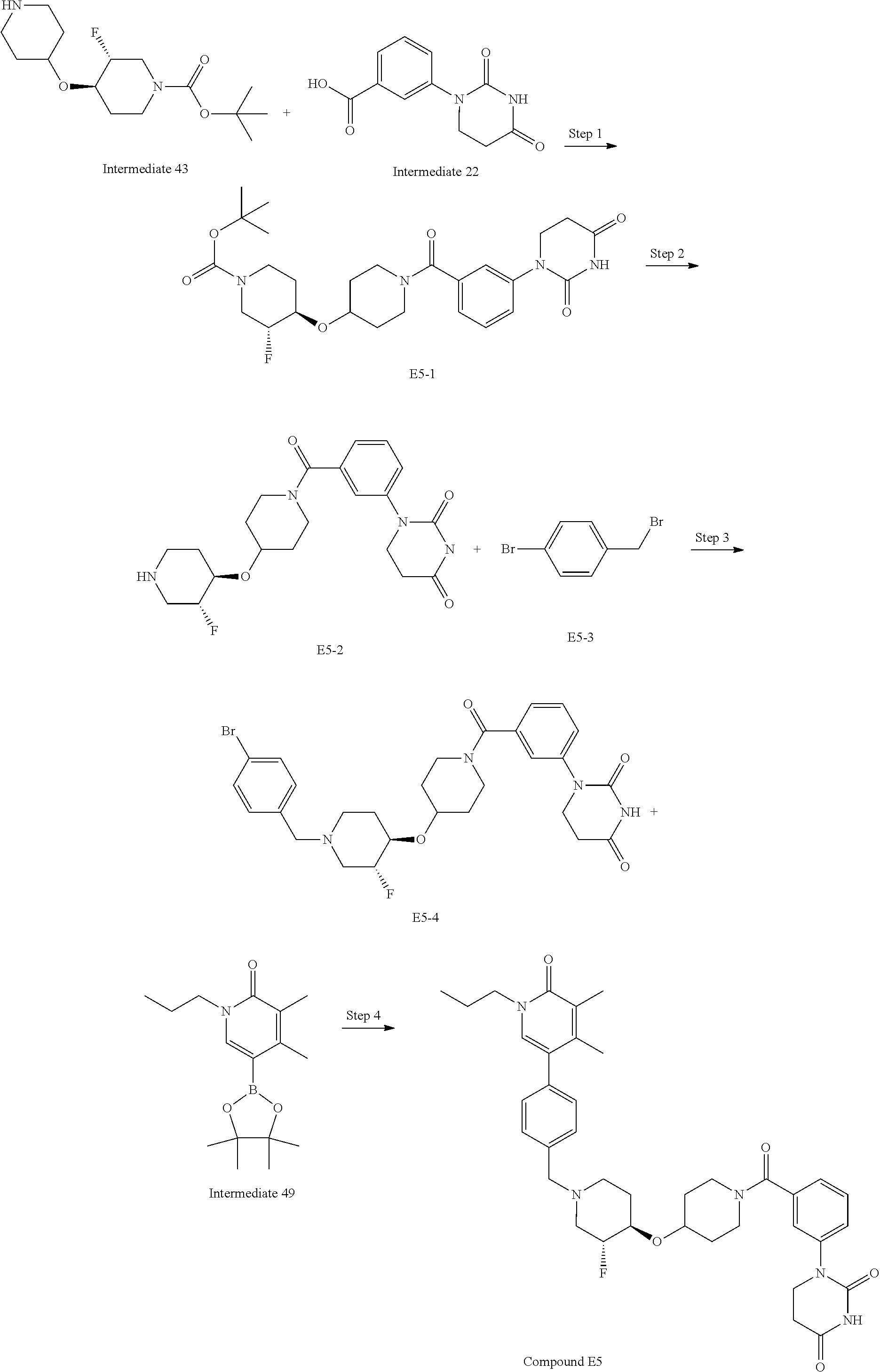

### Step 1: tert-butyl (3R,4R)-4-((1-(3-(2,4-dioxotetrahydropyrimidin-1(2H)-yl)benzoyl)piperidin-4-yl)oxy)-3-fluoropiperidine-1-carboxylate (E5-1)

To a solution of 3-(2,4-dioxotetrahydropyrimidin-1(2H)-yl)benzoic acid (Intermediate 22, 296 mg, 1.264 mmol) in DCM (8.5 ml) was added HATU (521 mg, 1.370 mmol) and the RM was stirred under a nitrogen atmosphere for 3 min. Tert-butyl (3R,4R)-3-fluoro-4-(piperidin-4-yloxy)piperidine-1-carboxylate 4-methylbenzenesulfonate salt (Intermediate 43, 500 mg, 1.054 mmol) was added, followed by DIEA (0.920 ml, 5.270 mmol) and the RM was stirred at RT for 1 h. The mixture was added into a mixture of water (3 ml) and an aq. sat. solution of NaHCO₃(3 ml) and stirred at RT for 5 min. The organic phase was washed with an aq. sat. solution of NaHCO₃ and brine, dried over MgSO₄ and concentrated. The residue was purified by chromatography on silica gel eluting with EtOAc (from 0% to 100%) in heptane, yielding the title compound tert-butyl (3R,4R)-4-((1-(3-(2,4-dioxotetrahydropyrimidin-1(2H)-yl)benzoyl)piperidin-4-yl)oxy)-3-fluoropiperidine-1-carboxylate, E5-1, as a solid (777 mg).

Method LCMS-ACQ-QDA #KAB0746-acidic: Rt=0.90 min; [M+H]⁺=519.

### Step 2: 1-(3-(4-(((3R,4R)-3-fluoropiperidin-4-yl)oxy)piperidine-1-carbonyl)phenyl)-dihydropyrimidine-2,4(1H,3H)-dione (E5-2)

To a solution of tert-butyl (3R,4R)-4-((1-(3-(2,4-dioxotetrahydropyrimidin-1(2H)-yl)benzoyl)piperidin-4-yl)oxy)-3-fluoropiperidine-1-carboxylate (E5-1, 777 mg, 1.504 mmol) in MeOH (1 ml) under a nitrogen atmosphere was added a solution of HCl (4 M) in 1,4-dioxane (9.36 ml) and the RM was stirred at RT for 1.5 h. The mixture was concentrated, yielding the title compound 1-(3-(4-(((3R,4R)-3-fluoropiperidin-4-yl)oxy)piperidine-1-carbonyl)phenyl)dihydropyrimidine-2,4(1H,3H)-dione, E5-2, as a solid hydrochloride salt (717 mg), which was directly used for the next step without further purification.

Method LCMS-ACQ-QDA #KAB0746—acidic: Rt=0.43 min; [M+H]⁺=419.

### Step 3: 1-(3-(4-(((3R,4R)-1-(4-bromobenzyl)-3-fluoropiperidin-4-yl)oxy)piperidine-1-carbonyl)phenyl)dihydropyrimidine-2,4(1H,3H)-dione (E5-4)

To a solution of 1-bromo-4-(bromomethyl)benzene (E5-3, 123 mg, 0.491 mmol) in ACN (3.6 ml) were added 1-(3-(4-(((3R,4R)-3-fluoropiperidin-4-yl)oxy)piperidine-1-carbonyl)phenyl)-dihydropyrimidine-2,4(1H,3H)-dione hydrochloride salt (E5-2, 180 mg, 0.396 mmol) and DIEA (1.04 ml, 5.94 mmol). The RM was stirred under a nitrogen atmosphere at 52° C. overnight. The mixture was concentrated and the residue was purified by chromatography on silica gel eluting with a mixture of DCM/MeOH/˜30% aq. NH₄OH (67:33:0.01) (from 0% to 30%) in DCM, yielding the title compound 1-(3-(4-(((3R,4R)-1-(4-bromobenzyl)-3-fluoropiperidin-4-yl)oxy)piperidine-1-carbonyl)phenyl)dihydropyrimidine-2,4(1H,3H)-dione, E5-4, as a solid (190 mg).

Method LCMS-ACQ-QDA #KAB0746—acidic: Rt=0.67 min; [M+H]⁺=587 and 589.

### Step 4: 1-(3-(4-(((3R,4R)-1-(4-(4,5-dimethyl-6-oxo-1-propyl-1,6-dihydropyridin-3-yl)benzyl)-3-fluoropiperidin-4-yl)oxy)piperidine-1-carbonyl)phenyl)dihydropyrimidine-2,4(1H,3H)-dione (Compound E5)

To a mixture of 3,4-dimethyl-1-propyl-5-(4,4,5,5-tetramethyl-1,3,2-dioxaborolan-2-yl)pyridin-2(1H)-one (Intermediate 49, 35.2 mg, 0.121 mmol), 1-(3-(4-(((3R,4R)-1-(4-bromobenzyl)-3-fluoropiperidin-4-yl)oxy)piperidine-1-carbonyl)phenyl)dihydropyrimidine-2,4(1H,3H)-dione (E5-4, 64.6 mg, 0.110 mmol) and solid Na₂CO₃ (35 mg, 0.330 mmol) in a mixture of 1,4-dioxane (1.1 ml) and water (0.275 ml) under a nitrogen atmosphere was added PdCl₂(dppf)-CH₂Cl₂ (19.8 mg, 0.024 mmol) and the RM was heated in a microwave oven at 100° C. for 65 min. The mixture was diluted with DCM (2 ml) and water (2 ml), the aq. phase was extracted with DCM and the combined organic phases were concentrated. The residue was purified by preparative HPLC on an XBridge C18 OBD column (50×30 mm, 5 μm) eluting with ACN (from 25% to 50% ) in an aq. solution of NH₄OH (5 mM), yielding the title compound 1-(3-(4-(((3R,4R)-1-(4-(4,5-dimethyl-6-oxo-1-propyl-1,6-dihydropyridin-3-yl)benzyl)-3-fluoropiperidin-4-yl)oxy)piperidine-1-carbonyl)phenyl)dihydropyrimidine-2,4(1H,3H)-dione, Compound E5, as a solid (9.8 mg).

Method LCMS-ACQ-QDA #KAB0746: Rt=0.98 min; [M+H]⁺=672.

¹H NMR (600 MHz, DMSO-d₆) δ[ppm] 10.41 (s, 1H), 7.47-7.38 (m, 3H), 7.37-7.29 (m, 3H), 7.28-7.19 (m, 3H), 4.50-4.27 (m, 1H), 4.06-3.90 (m, 1H), 3.89-3.79 (m, 4H), 3.79-3.72 (m, 1H), 3.60-3.44 (m, 4H), 3.18 (m, 1H), 3.04-2.92 (m, 1H), 2.71 (t, 2H), 2.69-2.63 (m, 1H), 2.22-1.72 (m, 12H), 1.71-1.59 (m, 2H), 1.56-1.33 (m, 3H), 0.86 (t, J=7.4 Hz, 3H).

### Compound E6: 1-(5-(4-(2-(4-(4-(2-butyl-1-oxo-1,2-dihydro-2,7-naphthyridin-4-yl)-2,6-difluorophenoxy)piperidin-1-yl)ethyl)piperidine-1-carbonyl)-2-methoxyphenyl)dihydropyrimidine-2,4(1H,3H)-dione

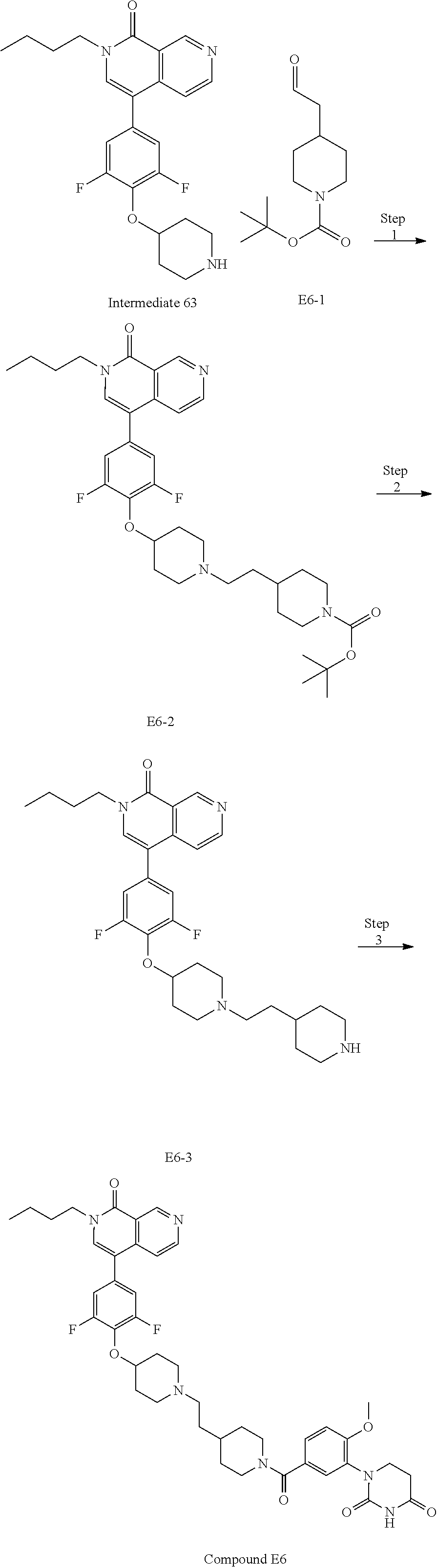

### Step 1: tert-Butyl 4-(2-(4-(4-(2-butyl-1-oxo-1,2-dihydro-2,7-naphthyridin-4-yl)-2,6-difluorophenoxy)piperidin-1-yl)ethyl)piperidine-1-carboxylate (E6-2)

To a mixture of 2-butyl-4-(3,5-difluoro-4-(piperidin-4-yloxy)phenyl)-2,7-naphthyridin-1(2H)-one (Intermediate 63, 85 mg, 0.162 mmol), TEA (0.100 ml, 0.717 mmol) and tert-butyl 4-(2-oxoethyl)piperidine-1-carboxylate (E6-1, 47 mg, 0.207 mmol) in MeOH (2 ml) under an argon atmosphere was added a solution of ZnCl₂ (0.7 M) in THE (0.300 ml, 0.210 mmol) and the RM was stirred at RT for 7 h. Solid NaBH₃CN (18 mg, 0.286 mmol) was added and the RM was stirred at RT for 16 h. The mixture was concentrated, yielding the title compound tert-butyl 4-(2-(4-(4-(2-butyl-1-oxo-1,2-dihydro-2,7-naphthyridin-4-yl)-2,6-difluorophenoxy)piperidin-1-yl)ethyl)piperidine-1-carboxylate, E6-2, as a solid (126 mg), which was used for the next step without further purification.

Method LCMS_MLG9: Rt=0.84 min; [M+H]⁺=625.

### Step 2: 2-Butyl-4-(3,5-difluoro-4-((1-(2-(piperidin-4-yl)ethyl)piperidin-4-yl)oxy)phenyl)-2,7-naphthyridin-1(2H)-one (E6-3)

To a solution of tert-butyl 4-(2-(4-(4-(2-butyl-1-oxo-1,2-dihydro-2,7-naphthyridin-4-yl)-2,6-difluorophenoxy)piperidin-1-yl)ethyl)piperidine-1-carboxylate (E6-2, 101 mg, 0.161 mmol) in DCM (1.5 ml) was added TFA (0.350 ml, 4.54 mmol) and the RM was stirred at RT for 1 h.

The mixture was concentrated and the residue was purified by reverse phase chromatography on an Isco REDISEP® Gold HP C18 column (15.5 g) eluting with ACN (from 2% to 100%) in an aq. solution of TFA (0.1%), yielding the title compound 2-butyl-4-(3,5-difluoro-4-((1-(2-(piperidin-4-yl)ethyl)piperidin-4-yl)oxy)phenyl)-2,7-naphthyridin-1(2H)-one, E6-3, as a solid TFA salt (94 mg).

Method LCMS_MLG9: Rt=0.56 min; [M+H]⁺=525.

### Step 3: 1-(5-(4-(2-(4-(4-(2-butyl-1-oxo-1,2-dihydro-2,7-naphthyridin-4-yl)-2,6-difluorophenoxy)piperidin-1-yl)ethyl)piperidine-1-carbonyl)-2-methoxyphenyl)dihydropyrimidine-2,4(1H,3H)-dione (Compound E6)

To a solution of 3-(2,4-dioxotetrahydropyrimidin-1(2H)-yl)-4-methoxybenzoic acid (Intermediate 25, 38 mg, 0.144 mmol) in DMF (0.5 ml) was added DIEA (0.070 ml, 0.401 mmol) and HATU (59 mg, 0.155 mmol) and the RM was stirred at RT for 30 min. A solution of 2-butyl-4-(3,5-difluoro-4-((1-(2-(piperidin-4-yl)ethyl)piperidin-4-yl)oxy)phenyl)-2,7-naphthyridin-1(2H)-one (E6-3, 94 mg, 0.119 mmol) and DIEA (0.070 ml, 0.401 mmol) in DMF (0.5 ml) was added and the RM was stirred at RT for 2 h. The mixture was purified by reverse phase chromatography on an Isco REDISEP® Gold HP C18 column (15.5 g) eluting with ACN (from 2% to 100%) in an aq. solution of TFA (0.1%). Fractions containing the title compound were combined and concentrated, dissolved in DMSO and purified by reverse phase chromatography on an Isco REDISEP® Gold HP C18 column (15.5 g) eluting with ACN (from 2% to 100%) in an aq. solution of TFA (0.1%), yielding the title compound 1-(5-(4-(2-(4-(4-(2-butyl-1-oxo-1,2-dihydro-2,7-naphthyridin-4-yl)-2,6-difluorophenoxy)piperidin-1-yl)ethyl)piperidine-1-carbonyl)-2-methoxyphenyl)dihydropyrimidine-2,4(1H,3H)-dione, Compound E6, as a solid (46 mg).

Method LCMS_MLG2: Rt=0.67 min; [M+H]⁺=772.

¹H NMR (600 MHz, DMSO-d₆) δ[ppm] 10.33 (s, 1H), 9.43 (d, J=0.8 Hz, 1H), 8.73 (d, J=5.6 Hz, 1H), 7.88 (s, 1H), 7.50 (dd, J=5.7, 0.9 Hz, 1H), 7.36 (dd, J=8.5, 2.2 Hz, 1H), 7.32 (m, 3H), 7.15 (d, J=8.6 Hz, 1H), 4.39 (m, 1H), 4.21 (m, 1H), 4.02 (t, J=7.4 Hz, 2H), 3.84 (m, 4H), 3.60 (t, J=6.7 Hz, 2H), 2.69 (m, 6H), 2.32 (t, J=7.4 Hz, 2H), 2.15 (m, 2H), 1.93 (m, 2H), 1.82-1.51 (m, 7H), 1.40 (m, 2H), 1.33 (m, 2H), 1.15-1.05 (m, 2H), 0.92 (t, J=7.4 Hz, 3H).

### Compound E7: 1-((1-(2-(4-(((3R,4R)-1-(4-(4,5-Dimethyl-6-oxo-1-propyl-1,6-dihydropyridin-3-yl)-2,6-dimethoxyphenethyl)-3-fluoropiperidin-4-yl)oxy)piperidin-1-yl)ethyl)-2-oxo-1,2-dihydropyridin-3-yl)methyl)dihydropyrimidine-2,4(1H,3H)-dione

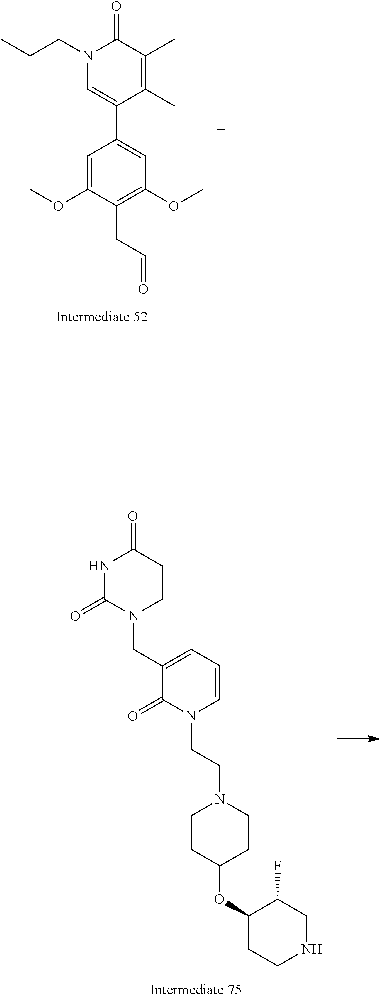 [SMILES: CCCn1cc(-c2cc(OC)c(CC=O)c(OC)c2)c(C)c(C)c1=O ; O=C1CCN(Cc2cccn(CCN3CCC(O[C@@H]4CCNC[C@H]4F)CC3)c2=O)C(=O)N1]

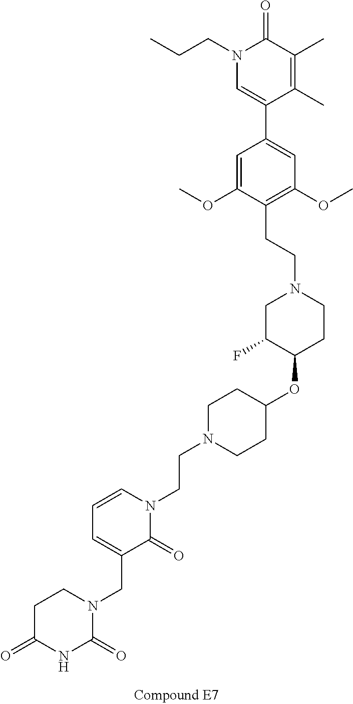 [SMILES: CCCn1cc(-c2cc(OC)c(CCN3CC[C@@H](OC4CCN(CCn5cccc(CN6CCC(=O)NC6=O)c5=O)CC4)[C@H](F)C3)c(OC)c2)c(C)c(C)c1=O]

To a mixture of 1-((1-(2-(4-(((3R,4R)-3-fluoropiperidin-4-yl)oxy)piperidin-1-yl)ethyl)-2-oxo-1,2-dihydropyridin-3-yl)methyl)dihydropyrimidine-2,4(1H,3H)-dione TFA salt (Intermediate 75, 97 mg, 0.135 mmol), 2-(4-(4,5-dimethyl-6-oxo-1-propyl-1,6-dihydropyridin-3-yl)-2,6-dimethoxyphenyl)acetaldehyde (Intermediate 52, 50 mg, 0.140 mmol) and TEA (0.060 ml, 0.430 mmol) in a mixture of MeOH (1.5 ml) and DCM (1.5 ml) was added a solution of ZnCl₂ (0.7 M) in THE (0.300 ml, 0.210 mmol) and the RM was stirred at RT for 18 h. Solid NaBH₃CN (17 mg, 0.271 mmol) was added and the RM was stirred at RT overnight. The mixture was concentrated and the residue was purified by reverse phase chromatography on a REDISEP® Gold HP C18 column (15.5 g) eluting with ACN (from 0% to 100%) in an aq. solution of TFA (0.1%). Fractions containing the title compound were combined, concentrated and the residue was purified by reverse phase chromatography on a REDISEP® Gold HP C18 column (15.5 g) eluting with ACN (from 1% to 100%) in an aq. solution of NH₄HCO₃ (0.1%), yielding the title compound 1-((1-(2-(4-(((3R,4R)-1-(4-(4,5-dimethyl-6-oxo-1-propyl-1,6-dihydropyridin-3-yl)-2,6-dimethoxyphenethyl)-3-fluoropiperidin-4-yl)oxy)piperidin-1-yl)ethyl)-2-oxo-1,2-dihydropyridin-3-yl)methyl)dihydropyrimidine-2,4(1H,3H)-dione, Compound E7, as a solid (50 mg).

Method LCMS_MLG4: Rt=2.03 min; [M+H]⁺=777.

¹H NMR (600 MHz, DMSO-d₆) δ[ppm] 10.15 (s, 1H), 7.58 (m, 1H), 7.44 (s, 1H), 7.33-7.19 (m, 1H), 6.50 (s, 2H), 6.20 (m, 1H), 4.31 (m, 3H), 3.99 (s, 2H), 3.81 (m, 8H), 3.44 (m, 4H), 3.08 (m, 1H), 2.73 (m, 4H), 2.80-2.67 (m, 5H), 2.46-2.29 (m, 3H), 2.20-2.09 (m, 3H), 2.08-2.01 (m, 6H), 1.93-1.62 (m, 5H), 1.38 (m, 3H), 0.86 (m, 3H).

### Compound E12: 1-(5-(4-((1-(4-(4,5-dimethyl-6-oxo-1-propyl-1,6-dihydropyridin-3-yl)-2,6-difluorophenethyl)piperidin-4-yl)oxy)piperidine-1-carbonyl)-2-methoxyphenyl)dihydropyrimidine-2,4(1H,3H)-dione

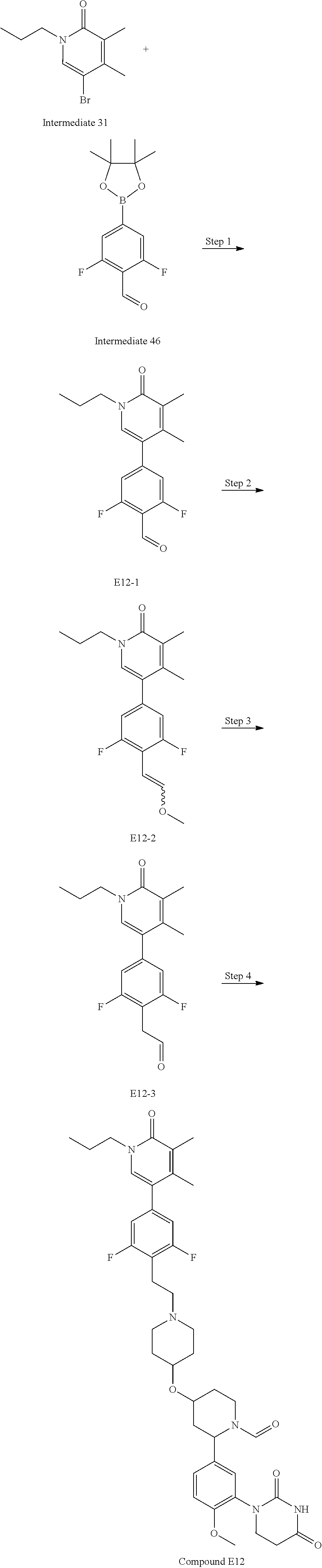 [SMILES: CCCn1cc(Br)c(C)c(C)c1=O ; CC1(C)OB(c2cc(F)c(C=O)c(F)c2)OC1(C)C ; CCCn1cc(-c2cc(F)c(C=O)c(F)c2)c(C)c(C)c1=O ; CCCn1cc(-c2cc(F)c(C=COC)c(F)c2)c(C)c(C)c1=O ; CCCn1cc(-c2cc(F)c(CC=O)c(F)c2)c(C)c(C)c1=O ; CCCn1cc(-c2cc(F)c(CCN3CCC(OC4CCN(C=O)C(c5ccc(OC)c(N6CCC(=O)NC6=O)c5)C4)CC3)c(F)c2)c(C)c(C)c1=O]

### Step 1: 4-(4,5-dimethyl-6-oxo-1-propyl-1,6-dihydropyridin-3-yl)-2,6-difluorobenzaldehyde (E12-1)

To a solution of 5-bromo-3,4-dimethyl-1-propylpyridin-2(1H)-one (Intermediate 31, 1 g, 4.18 mmol), 2,6-difluoro-4-(4,4,5,5-tetramethyl-1,3,2-dioxaborolan-2-yl)benzaldehyde (Intermediate 46, 2.2 g, 8.19 mmol) and K₂CO₃ (1.13 g, 8.19 mmol) in a mixture of 1,4-dioxane (10 ml) and water (1 ml) under a nitrogen atmosphere was added Pd(dppf)Cl₂ (305.8 mg, 0.418 mmol) and the RM was stirred at 80° C. for 16 h. The mixture was added into water (30 ml), the aq. phase was extracted with EtOAc (3×15 ml), the combined organic phases were washed with brine (2×20 ml), dried over Na₂SO₄ and concentrated. The residue was purified by chromatography on silica gel eluting with EtOAc (from 0% to 100%) in PE, yielding the title compound 4-(4,5-dimethyl-6-oxo-1-propyl-1,6-dihydropyridin-3-yl)-2,6-difluorobenzaldehyde, E12-1, as a solid (1.2 g).

Method LCMS WX2: Rt=0.88 min; [M+H]⁺=306.1.

### Step 2: (E,Z)-5-(3,5-difluoro-4-(2-methoxyvinyl)phenyl)-3,4-dimethyl-1-propylpyridin-2(1H)-one (E12-2)

To a solution of chloro(methoxymethyl)triphenylphosphorane (1.02 g, 2.98 mmol) in THE (5 ml) was added a solution of t-BuOK (334.45 mg, 2.98 mmol) in THF (5 ml) at 0° C. and the RM was stirred at 0° C. for 2 h. A solution of 4-(4,5-dimethyl-6-oxo-1-propyl-1,6-dihydropyridin-3-yl)-2,6-difluorobenzaldehyde (E12-1, 700 mg, 2.29 mmol) in THE (5 ml) was added and the RM was stirred at 70° C. for 2 h. The mixture was concentrated and the residue was purified by preparative HPLC on a Phenomenex Luna C18 column (40×150 mm, 15 μm) eluting with ACN (from 10% to 40%) in an aq. solution of TFA (0.1%), yielding the title compounds (E,Z)-5-(3,5-difluoro-4-(2-methoxyvinyl)phenyl)-3,4-dimethyl-1-propylpyridin-2(1H)-one, E12-2, as a solid (500 mg).

Method LCMS WX2: Rt=0.96 min; [M+H]⁺=334.

### Step 3: 2-(4-(4,5-dimethyl-6-oxo-1-propyl-1,6-dihydropyridin-3-yl)-2,6-difluorophenyl)acetaldehyde (E12-3)

To a solution of (E,Z)-5-(3,5-difluoro-4-(2-methoxyvinyl)phenyl)-3,4-dimethyl-1-propylpyridin-2(1H)-one (E12-2, 50 mg, 0.15 mmol) in acetone (1 ml) was added an aq. solution of HCl (2 M, 1 ml, 2 mmol) and the RM was stirred at 40° C. for 12 h. The mixture was concentrated, water (10 ml) was added, the aq. phase was extracted with DCM (3×10 ml), the combined organic phases were washed with brine (2×5 ml), dried over Na₂SO₄ and concentrated to afford the title compound 2-(4-(4,5-dimethyl-6-oxo-1-propyl-1,6-dihydropyridin-3-yl)-2,6-difluorophenyl)acetaldehyde, E12-3, as an oil (60 mg), which was used for the next step without further purification.

Method LCMS WX3: Rt=0.97 min; [M+H]⁺=320.3.

### Step 4: 1-(5-(4-((1-(4-(4,5-dimethyl-6-oxo-1-propyl-1,6-dihydropyridin-3-yl)-2,6-difluorophenethyl)piperidin-4-yl)oxy)piperidine-1-carbonyl)-2-methoxyphenyl)dihydropyrimidine-2,4(1H,3H)-dione (Compound E12)

To a solution of 2-(4-(4,5-dimethyl-6-oxo-1-propyl-1,6-dihydropyridin-3-yl)-2,6-difluorophenyl)acetaldehyde (E12-3, 60 mg) and 1-(2-methoxy-5-(4-(piperidin-4-yloxy)piperidine-1-carbonyl)phenyl)dihydropyrimidine-2,4(1H,3H)-dione (Intermediate 29, 80 mg, 0.188 mmol) in a mixture of THF (1 ml) and EtOH (1 ml) were added DIEA (24.30 mg, 0.188 mmol) and a solution of ZnCl₂ (1 M) in TH (0.37 ml, 0.376 mmol). The RM was stirred at 25° C. for 1 h, solid NaBH₃CN (23.61 mg, 0.376 mmol) was added and the RM was stirred at 25° C. for 15 h. The mixture was added into MeOH (15 ml), filtered and the filtrate was concentrated. The residue was purified by preparative HPLC on a Waters XBridge column (150×25 mm, 5 μm) eluting with ACN (from 10% to 50%) in an aq. solution of NH₄OH (0.1%), yielding the title compound 1-(5-(4-((1-(4-(4,5-dimethyl-6-oxo-1-propyl-1,6-dihydropyridin-3-yl)-2,6-difluorophenethyl)piperidin-4-yl)oxy)piperidine-1-carbonyl)-2 methoxyphenyl)dihydropyrimidine-2,4(1H,3H)-dione, Compound E12, as a solid (33.82 mg).

Method LCMS WX2: Rt=0.798 min; [M+H]⁺=734.4.

¹H NMR (400 MHz, DMSO-d₆) δ[ppm] 10.33 (s, 1H), 7.55-7.51 (m, 1H), 7.41-7.36 (m, 1H), 7.36-7.33 (m, 1H), 7.19-7.13 (m, 1H), 7.08-6.99 (m, 2H), 3.88-3.83 (m, 5H), 3.74-3.63 (m, 2H), 3.62-3.58 (m, 2H), 3.51-3.40 (m, 2H), 3.26-3.16 (m, 4H), 2.82-2.73 (m, 4H), 2.71-2.67 (m, 2H), 2.23-2.08 (m, 2H), 2.08-2.00 (m, 6H), 1.88-1.72 (m, 4H), 1.72-1.61 (m, 2H), 1.51-1.35 (m, 4H), 0.90-0.84 (m, 3H).

### Compound E15: 1-(2-fluoro-5-(4-((1-(4-(2-hexyl-1-oxo-1,2-dihydro-2,7-naphthyridin-4-yl)-2,6-dimethoxyphenethyl)piperidin-4-yl)oxy)piperidine-1-carbonyl)phenyl)dihydropyrimidine-2,4(1H,3H)-dione

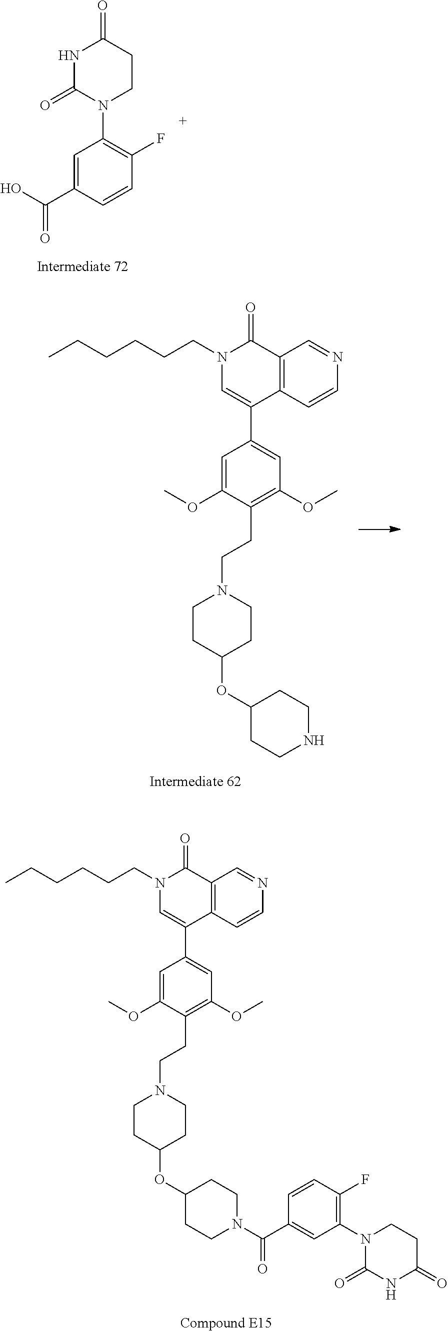

To a solution of 3-(2,4-dioxotetrahydropyrimidin-1(2H)-yl)-4-fluorobenzoic acid (Intermediate 72.18 mg, 0.071 mmol) in DMF (0.5 ml) were added DIEA (0.050 ml, 0.286 mmol) and HATU (27 mg, 0.071 mmol) and the RM was stirred at RT for 30 min. A solution of 4-(3,5-dimethoxy-4-(2-(4-(piperidin-4-yloxy)piperidin-1-yl)ethyl)phenyl)-2-hexyl-2,7-naphthyridin-1(2H)-one TFA salt (Intermediate 62, 49 mg, 0.060 mmol) and DIEA (0.050 ml, 0.286 mmol) in DMF (0.5 ml) was added and the RM was stirred at RT for 2 h. The mixture was purified by reverse phase chromatography on an Isco REDISEP® Gold HP C18 column (15.5 g) eluting with ACN (from 2% to 100%) in an aq. solution of NH₄HCO₃ (0.1%), yielding the title compound 1-(2-fluoro-5-(4-((1-(4-(2-hexyl-1-oxo-1,2-dihydro-2,7-naphthyridin-4-yl)-2,6-dimethoxyphenethyl)piperidin-4-yl)oxy)piperidine-1-carbonyl)phenyl)dihydropyrimidine-2,4(1H,3H)-dione, Compound E15, as a solid (40 mg).

Method LCMS_MLG8: Rt=0.81 min; [M+H]⁺=811.

¹H NMR (400 MHz, DMSO-d₆) δ[ppm] 10.52 (s, 1H), 9.44 (s, 1H), 8.71 (d, J=5.7 Hz, 1H), 7.82 (s, 1H), 7.59-7.49 (m, 2H), 7.43-7.33 (m, 2H), 6.68 (s, 2H), 4.02 (t, J=7.4 Hz, 2H), 3.80 (s, 6H), 3.79-3.67 (m, 3H), 3.60-3.37 (m, 3H), 3.25 (m, 2H), 2.83-2.69 (m, 6H), 2.39-2.29 (m, 2H), 2.10 (m, 2H), 1.93-1.65 (m, 6H), 1.53-1.37 (m, 4H), 1.37-1.26 (m, 6H), 0.91-0.79 (m, 3H).

### Compound E17: 1-((1-(2-(4-(((3R,4R)-4-(4-(2-butyl-1-oxo-1,2-dihydro-2,7-naphthyridin-4-yl)-2-fluorophenoxy)-3-fluoropiperidin-1-yl)methyl)piperidin-1-yl)ethyl)-2-oxo-1,2-dihydropyridin-3-yl)methyl)dihydropyrimidine-2,4(1H,3H)-dione

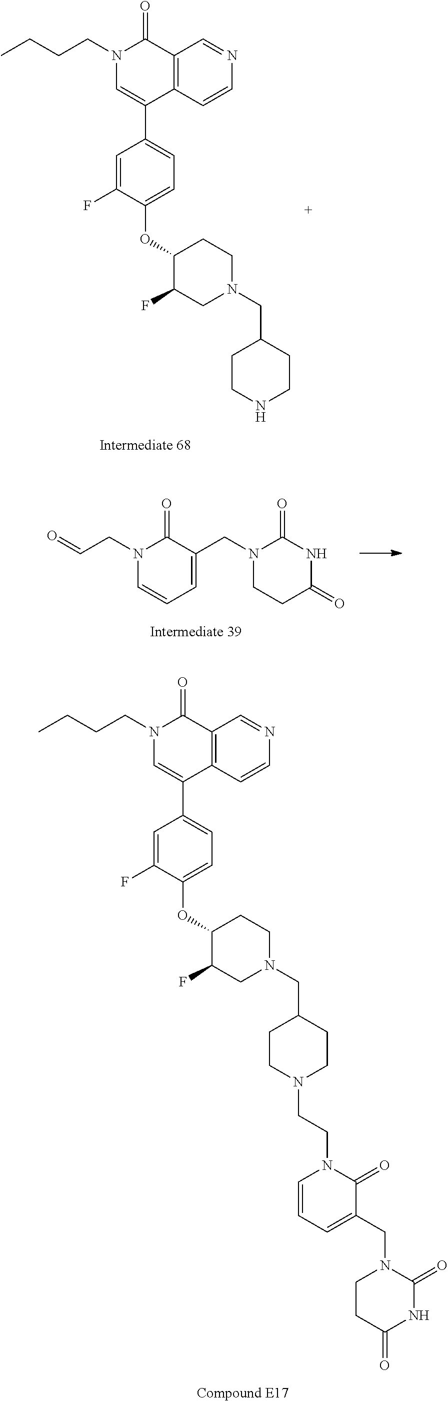 [SMILES: CCCCn1cc(-c2ccc(O[C@@H]3CCN(CC4CCNCC4)C[C@H]3F)c(F)c2)c2ccncc2c1=O ; O=CCn1cccc(CN2CCC(=O)NC2=O)c1=O ; CCCCn1cc(-c2ccc(O[C@@H]3CCN(CC4CCN(CCn5cccc(CN6CCC(=O)NC6=O)c5=O)CC4)C[C@H]3F)c(F)c2)c2ccncc2c1=O]

To a mixture of 2-butyl-4-(3-fluoro-4-(((3R,4R)-3-fluoro-1-(piperidin-4-ylmethyl)piperidin-4-yl)oxy)phenyl)-2,7-naphthyridin-1(2H)-one TFA salt (Intermediate 68, 250 mg, 0.196 mmol), 2-(3-((2,4-dioxotetrahydropyrimidin-1(2H)-yl)methyl)-2-oxopyridin-1(2H)-yl)acetaldehyde (Intermediate 39, 86 mg, 0.294 mmol) and TEA (0.100 ml, 0.717 mmol) in a mixture of MeOH (1.5 ml) and DCM (1.5 ml) was added a solution of ZnCl₂ (0.7 M) in THE (0.500 ml, 0.350 mmol) and the RM was stirred at RT for 18 h. Solid NaBH₃CN (24 mg, 0.382 mmol) was added and the RM was stirred at RT overnight. The mixture was concentrated and purified by reverse phase chromatography on a REDISEP® Gold HP C18 column (15.5 g) eluting with ACN (from 0% to 100%) in an aq. solution of TFA (0.1%). Fractions containing the title compound were combined, concentrated and the residue was purified by reverse phase chromatography on a REDISEP® Gold HP C18 column (15.5 g) eluting with ACN (from 1% to 100%) in an aq. solution of NH₄HCO₃ (0.1%), yielding the title compound 1-((1-(2-(4-(((3R,4R)-4-(4-(2-butyl-1-oxo-1,2-dihydro-2,7-naphthyridin-4-yl)-2-fluorophenoxy)-3-fluoropiperidin-1-yl)methyl)piperidin-1-yl)ethyl)-2-oxo-1,2-dihydropyridin-3-yl)methyl)dihydropyrimidine-2,4(1H,3H)-dione, Compound E17, as a solid (90 mg).

Method LCMS_MLG5: Rt=4.28 min; [M+H]⁺=758.

¹H NMR (600 MHz, DMSO-d₆) δ[ppm] 10.15 (s, 1H), 9.43 (s, 1H), 8.72 (d, J=5.6 Hz, 1H), 7.81 (s, 1H), 7.58 (d, J=6.7 Hz, 1H), 7.47-7.33 (m, 3H), 7.27 (d, J=6.8 Hz, 1H), 7.22 (dd, J=8.2, 2.2 Hz, 1H), 6.20 (t, J=6.8 Hz, 1H), 4.68 (m, 1H), 4.52 (m, 1H), 4.27 (s, 2H), 4.01 (m, 4H), 3.41 (t, J=6.8 Hz, 2H), 3.14-3.04 (m, 1H), 2.87 (m, 2H), 2.73 (m, 1H), 2.59-2.52 (m, 4H), 2.26-2.17 (m, 3H), 2.17-2.10 (m, 2H), 1.96 (m, 2H), 1.75-1.56 (m, 5H), 1.47 (m, 1H), 1.33 (m, 2H), 1.05 (m, 2H), 0.92 (t, J=7.4 Hz, 3H).

### Compound E22: 1-(5-(2-(4-(((3S,4S)-4-(4-(2-butyl-1-oxo-1,2-dihydro-2,7-naphthyridin-4-yl)-2-methoxyphenoxy)-3-fluoropiperidin-1-yl)methyl)piperidin-1-yl)ethoxy)-2-methylphenyl)dihydropyrimidine-2,4(1H,3H)-dione

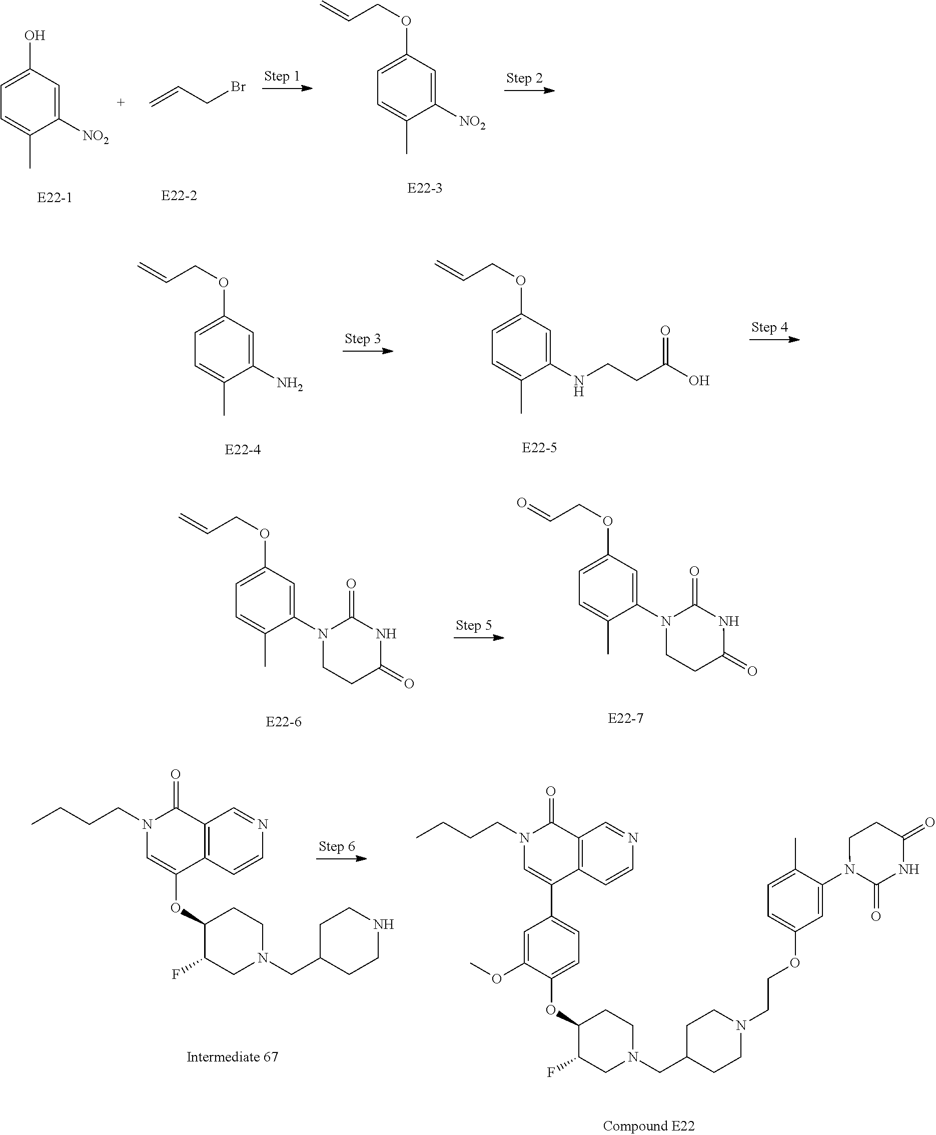

### Step 1: 4-(allyloxy)-1-methyl-2-nitrobenzene (E22-3)

To a mixture of 4-methyl-3-nitrophenol (E22-1, 15.3 g, 100 mmol) and K₂CO₃ (27.8 g, 200 mmol) in ACN (100 ml) was added allyl bromide (E22-2, 15.5 g, 130 mmol) and the RM was stirred at RT for 16 h. The mixture was filtered and the filtrate was concentrated, yielding the title compound 4-(allyloxy)-1-methyl-2-nitrobenzene, E22-3, as an oil (19 g), which was used for the next step without further purification.

Method LCMSA042: Rt=1.39 min; no MS signal observed.

### Step 2: 5-(allyloxy)-2-methylaniline (E22-4)

To a mixture of 4-(allyloxy)-1-methyl-2-nitrobenzene (E22-3, 19 g, 100 mmol) and Zn (39 g, 600 mmol) in EtOH (250 ml) was added AcOH (9 g, 75 mmol) and the RM was stirred at RT for 16 h. The mixture was filtered, the filtrate was concentrated and the residue was added into a mixture of EtOAc (500 ml), and water (200 ml). The pH of the mixture was adjusted to pH=9 by the addition of a sat. aq. solution of K₂CO₃, the organic phase was separated, dried over Na₂SO₄ and concentrated, yielding the title compound 5-(allyloxy)-2-methylaniline, E22-4, as a solid (17.4 g), which was used for the next step without further purification.

Method LCMS042: Rt=1.13 min; [M+H]⁺=164.

### Step 3: 3-((5-(allyloxy)-2-methylphenyl)amino)propanoic acid (E22-5)

To a solution of 5-(allyloxy)-2-methylaniline (E22-4, 17.4 g, 100 mmol) in toluene (50 ml) was added acrylic acid and the RM was stirred at 100° C. for 16 h. The solvent was removed, yielding the title compound 3-((5-(allyloxy)-2-methylphenyl)amino)propanoic acid, E22-5, as an oil (26 g), which was used for the next step without further purification.

Method LCMS042: Rt=0.78 min; [M+H]⁺=236.

### Step 4: 1-(5-(allyloxy)-2-methylphenyl)dihydropyrimidine-2,4(1H,3H)-dione (E22-6)

To a solution of 3-((5-(allyloxy)-2-methylphenyl)amino)propanoic acid (E22-5, 26 g, 100 mmol) in HOAc (500 ml) was added urea (48 g, 800 mmol) and the RM was stirred at 120° C. for 30 h. The mixture was concentrated, the residue was added into water (500 ml) and the pH of the mixture was adjusted to pH=7 by the addition of solid NaHCO₃. The solids were filtered and washed with water and MTBE, yielding the title compound 1-(5-(allyloxy)-2-methylphenyl)dihydropyrimidine-2,4(1H,3H)-dione, E22-6, as a solid (16 g).

Method LCMS042: Rt=1.05 min; [M+H]⁺=261.

### Step 5: 2-(3-(2,4-dioxotetrahydropyrimidin-1(2H)-yl)-4-methylphenoxy)acetaldehyde (E22-7)

To a solution of 1-(5-(allyloxy)-2-methylphenyl)dihydropyrimidine-2,4(1H,3H)-dione (E22-6, 8 g, 30 mmol) in DCM (200 ml) at −78° C. was added ozone gas for 20 min. The mixture was purged with nitrogen at −78° C. for 30 min, Me₂S (15 ml) was added and the mixture was stirred at −78° C. for 2 h. The mixture was concentrated and the residue was purified by chromatography on silica gel eluting with a mixture (1:3) of THE and DCM, yielding the title compound 2-(3-(2,4-dioxotetrahydropyrimidin-1(2H)-yl)-4-methylphenoxy)acetaldehyde, E22-7, as a solid (6.1 g).

Method LCMS042: Rt=0.65 min; [M+H]⁺=263.

¹H NMR (500 MHz, DMSO-d₆) δ[ppm] 10.34-10.30 (m, 1H) 9.67 (s, 1H) 7.18-7.16 (m, 1H) 6.97-6.75 (m, 2H) 4.84-4.64 (m, 2H) 3.77-3.75 (m, 1H) 2.74-2.64 (m, 2H) 2.10 (s, 3H).

### Step 6: 1-(5-(2-(4-(((3S,4S)-4-(4-(2-butyl-1-oxo-1,2-dihydro-2,7-naphthyridin-4-yl)-2-methoxyphenoxy)-3-fluoropiperidin-1-yl)methyl)piperidin-1-yl)ethoxy)-2-methylphenyl)dihydropyrimidine-2,4(1H,3H)-dione (Compound E22)

To a solution of 2-butyl-4-(4-(((3S,4S)-3-fluoro-1-(piperidin-4-ylmethyl)piperidin-4-yl)oxy)-3-methoxyphenyl)-2,7-naphthyridin-1(2H)-one TFA salt (Intermediate 67, 70 mg, 0.087 mmol) in MeOH (1 ml) under an argon atmosphere were added TEA (60 μl, 0.443 mmol), a solution of 2-(3-(2,4-dioxotetrahydropyrimidin-1(2H)-yl)-4-methylphenoxy)acetaldehyde (E22-7, 34.2 mg, 0.104 mmol) in MeOH (0.7 ml) and a solution of ZnCl₂ (0.5 M) in TH (0.191 ml, 0.095 mmol) and the RM was stirred at RT for 1 h. A solution of NaBH₃CN (1 M) in THE (139 μl, 0.139 mmol) was added dropwise, followed by DCM (1 ml), and the RM was stirred at RT for 15.5 h. The mixture was quenched with a mixture of water, ACN and a few drops of TFA, concentrated, adsorbed on ISOLUTE® HM-N and purified by reverse phase chromatography on a REDISEP® Gold HP C18 column (15.5 g) eluting with ACN (from 4.8% to 100%) in an aq. solution of TFA (0.1%), followed by a second purification by chromatography on reverse phase on a REDISEP® Gold HP C18 column (50 g) eluting with ACN (from 4.8% to 100%) in an aq. solution of TFA (0.1%). Fractions containing the title compound were combined, concentrated and the residue was purified by reverse phase chromatography on a REDISEP® Gold HP C18 column (15.5 g) eluting with ACN (from 1% to 100%) in an aq. solution of NH₄HCO₃ (0.1%) to afford two batches containing the title compound. The second batch was purified by reverse phase chromatography on a REDISEP® Gold HP C18 column (15.5 g) eluting with ACN (from 1% to 100%) in an aq. solution of NH₄HCO₃ (0.1%). The resulting material was combined with the first batch, dissolved in MeOH and purified by preparative HPLC on a Waters™ X-Bridge™ C18 OBD™ column (100×30 mm, 5 μm) eluting with ACN (from 30% to 100%) in an aq. solution of NH₄OH (7.3 mM), yielding the title compound 1-(5-(2-(4-(((3S,4S)-4-(4-(2-butyl-1-oxo-1,2-dihydro-2,7-naphthyridin-4-yl)-2-methoxyphenoxy)-3-fluoropiperidin-1-yl)methyl)piperidin-1-yl)ethoxy)-2-methylphenyl)dihydropyrimidine-2,4(1H,3H)-dione, Compound E22, as a solid (15 mg).

Method LCMS_MLG3: Rt=1.10 min; [M+H]⁺=769.

¹H NMR (600 MHz, DMSO-d₆) δ[ppm] 10.31 (s, 1H), 9.43 (d, J=0.9 Hz, 1H), 8.71 (d, J=5.6 Hz, 1H), 7.77 (s, 1H), 7.48 (dd, J=5.6, 0.9 Hz, 1H), 7.20 (d, J=8.4 Hz, 1H), 7.16 (d, J=8.5 Hz, 1H), 7.07 (d, J=2.1 Hz, 1H), 6.95 (dd, J=8.2, 2.1 Hz, 1H), 6.90 (d, J=2.7 Hz, 1H), 6.83 (dd, J=8.3, 2.7 Hz, 1H), 4.76-4.53 (m, 1H), 4.44-4.34 (m, 1H), 4.06-3.96 (m, 4H), 3.82 (s, 3H), 3.80-3.67 (m, 1H), 3.56-3.47 (m, 1H), 3.12-3.01 (m, 1H), 2.95-2.83 (m, 2H), 2.77-2.64 (m, 5H), 2.24-2.17 (m, 3H), 2.16-2.10 (m, 2H), 2.09 (s, 3H), 2.05-1.96 (m, 2H), 1.75-1.63 (m, 4H), 1.62-1.53 (m, 1H), 1.52-1.43 (m, 1H), 1.38-1.30 (m, 2H), 1.14-1.05 (m, 2H), 0.92 (t, J=7.4 Hz, 3H).

### Compound E23: 1-((1-(2-(4-((1-(4-(4,5-dimethyl-6-oxo-1-propyl-1,6-dihydropyridin-3-yl)-2,6-dimethoxyphenethyl)piperidin-4-yl)oxy)piperidin-1-yl)ethyl)-2-oxo-1,2-dihydropyridin-3-yl)methyl)dihydropyrimidine-2,4(1H,3H)-dione

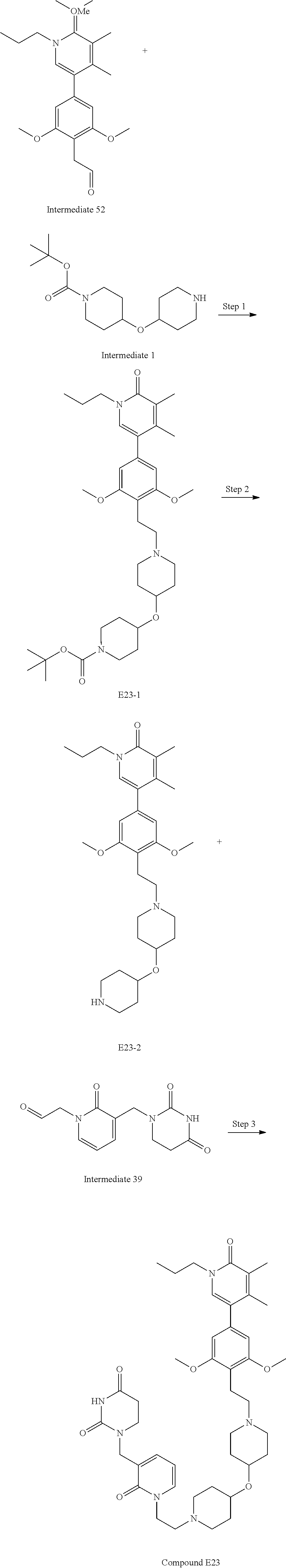

### Step 1: tert-butyl 4-((1-(4-(4,5-dimethyl-6-oxo-1-propyl-1,6-dihydropyridin-3-yl)-2,6-dimethoxyphenethyl)piperidin-4-yl)oxy)piperidine-1-carboxylate (E23-1)

To a mixture of 2-(4-(4,5-dimethyl-6-oxo-1-propyl-1,6-dihydropyridin-3-yl)-2,6-dimethoxyphenyl)acetaldehyde (Intermediate 52, 150 mg, 0.393 mmol), TEA (0.150 ml, 1.076 mmol) and tert-butyl 4-(piperidin-4-yloxy)piperidine-1-carboxylate (Intermediate 1, 200 mg, 0.703 mmol) in MeOH (4 ml) was added a solution of ZnCl₂ (0.7 M) in THF (1.0 ml, 0.700 mmol) and the RM was stirred under an argon atmosphere at RT for 24 h. Solid NaBH₃CN (49 mg, 0.780 mmol) was added and the RM was stirred at RT for 20 h. The mixture was concentrated, yielding the title compound tert-butyl 4-((1-(4-(4,5-dimethyl-6-oxo-1-propyl-1,6-dihydropyridin-3-yl)-2,6-dimethoxyphenethyl)piperidin-4-yl)oxy)piperidine-1-carboxylate, E23-1, as a solid (481 mg), which was used for the next step without further purification.

Method LCMS_IJ1: Rt=1.07; [M+H]⁺=612.

### Step 2: 5-(3,5-dimethoxy-4-(2-(4-(piperidin-4-yloxy)piperidin-1-yl)ethyl)phenyl)-3,4-dimethyl-1-propylpyridin-2(1H)-one (E23-2)

To a solution of tert-butyl 4-((1-(4-(4,5-dimethyl-6-oxo-1-propyl-1,6-dihydropyridin-3-yl)-2,6-dimethoxyphenethyl)piperidin-4-yl)oxy)piperidine-1-carboxylate (E23-1, 481 mg, 0.393 mmol) in DCM (4 ml) was added TFA (1.00 ml, 12.98 mmol) and the RM was stirred at RT for 1 h. The mixture was concentrated and the residue was purified by reverse phase chromatography on a REDISEP® C18 column (15.5 g) eluting with ACN (from 0% to 100%) in an aq. solution of TFA (0.1%), yielding the title compound 5-(3,5-dimethoxy-4-(2-(4-(piperidin-4-yloxy)piperidin-1-yl)ethyl)phenyl)-3,4-dimethyl-1-propylpyridin-2(1H)-one, E23-2, as a TFA salt (199 mg).

Method LCMS_MLG3: Rt=1.10 min; [M+H]⁺=512.

### Step 3: 1-((1-(2-(4-((1-(4-(4,5-dimethyl-6-oxo-1-propyl-1,6-dihydropyridin-3-yl)-2,6-dimethoxyphenethyl)piperidin-4-yl)oxy)piperidin-1-yl)ethyl)-2-oxo-1,2-dihydropyridin-3-yl)methyl)dihydropyrimidine-2,4(1H,3H)-dione (Compound E23)

A mixture of NaOAc (25 mg, 0.305 mmol), HOAc (18 μl, 0.314 μmol) and 5-(3,5-dimethoxy-4-(2-(4-(piperidin-4-yloxy)piperidin-1-yl)ethyl)phenyl)-3,4-dimethyl-1-propylpyridin-2(1H)-one TFA salt (E23-2, 199 mg, 0.105 mmol) in DCM (2 ml) was stirred at RT for 10 min. 2-(3-((2,4-Dioxotetrahydropyrimidin-1(2H)-yl)methyl)-2-oxopyridin-1(2H)-yl)acetaldehyde (Intermediate 39, 40 mg, 0.137 mmol) was added, followed by DMF (1 ml) and the RM was stirred at RT for 1 h. Solid NaBH(OAc)₃ (44 mg, 0.208 mmol) was added and the RM was stirred at RT for 20 h. The mixture was concentrated and the residue was purified by reverse phase chromatography on a REDISEP® C18 column (15.5 g) eluting with ACN (from 0% to 100%) in an aq. solution of TFA (0.1%). Fractions containing the title compound were combined, concentrated and the residue was purified by reverse phase chromatography on an X-Bridge® C18 column (250×50 mm, 5 m) eluting with ACN (from 10% to 40%) in an aq. solution of TFA (0.1%). Fractions containing the title compound were combined, filtered through a PL-HCO₃ MP SPE cartridge and the combined filtrates were freeze-dried, yielding the title compound 1-((1-(2-(4-((1-(4-(4,5-dimethyl-6-oxo-1-propyl-1,6-dihydropyridin-3-yl)-2,6-dimethoxyphenethyl)piperidin-4-yl)oxy)piperidin-1-yl)ethyl)-2-oxo-1,2-dihydropyridin-3-yl)methyl)dihydropyrimidine-2,4(1H,3H)-dione, Compound E23, as a solid (8.7 mg).

Method LCMS_MLG4: Rt=2.06 min; [M+H]⁺=759.

### Compound E24: 1-((1-(2-(4-((1-(2-fluoro-6-methoxy-4-(2-methyl-1-oxo-1,2-dihydro-2,7-naphthyridin-4-yl)benzyl)piperidin-4-yl)oxy)piperidin-1-yl)ethyl)-2-oxo-1,2-dihydropyridin-3-yl)methyl)dihydropyrimidine-2,4(1H,3H)-dione

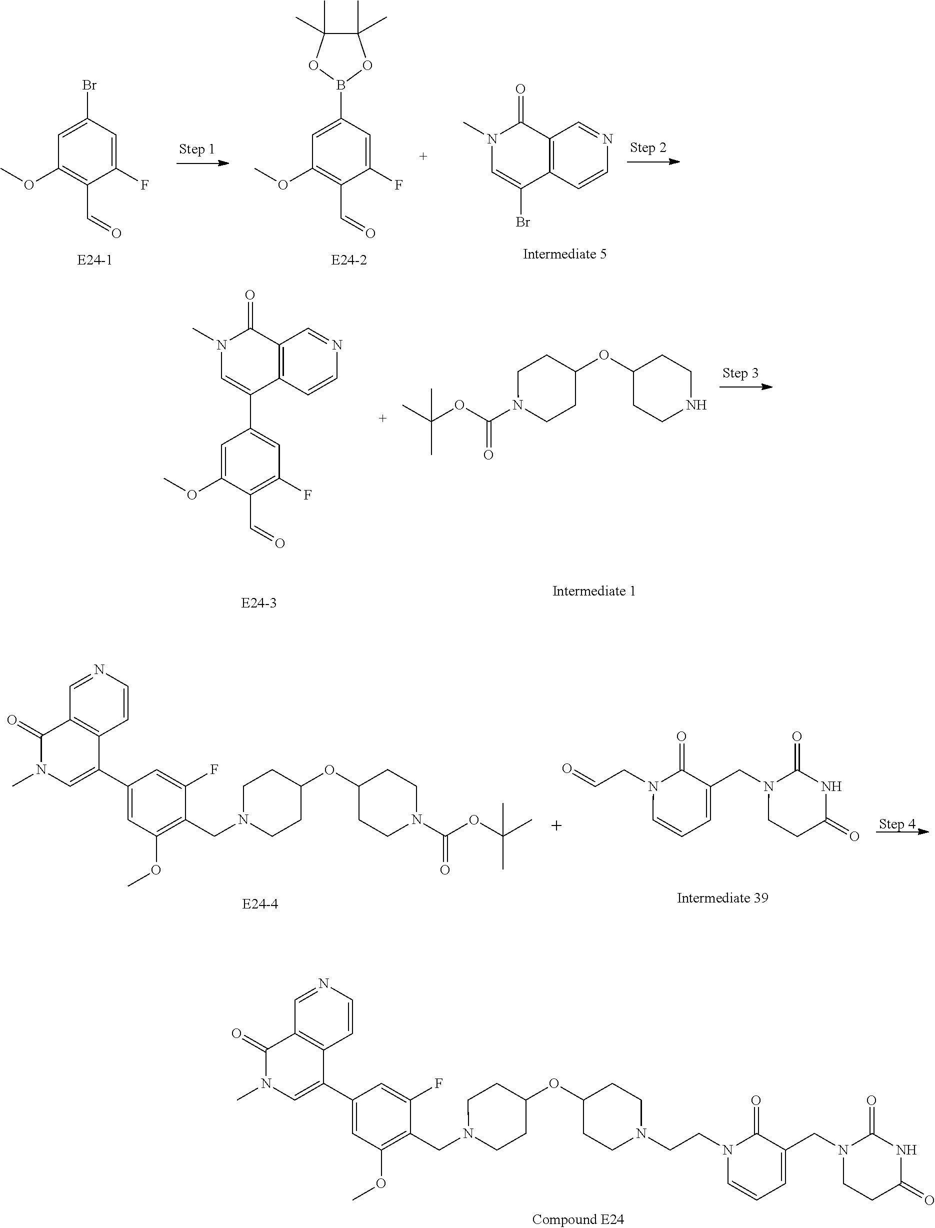

### Step 1: 2-fluoro-6-methoxy-4-(4,4,5,5-tetramethyl-1,3,2-dioxaborolan-2-yl)benzaldehyde (E24-2)

To a mixture of 4-bromo-2-fluoro-6-methoxybenzaldehyde (E24-1, 1.037 g, 4.45 mmol), BISPIN (1.400 g, 5.51 mmol) and KOAc (1.310 g, 13.35 mmol) in 1,4-dioxane (22 ml) under an argon atmosphere was added PdCl₂(dppf) (326 mg, 0.445 mmol) and the RM was stirred at 105° C. for 2.5 h. The mixture was cooled to RT, diluted with EtOAc and filtered over CELITE®. The filtrate was concentrated, the residue was adsorbed on silica gel and purified by chromatography on silica gel eluting with EtOAc (from 0% to 100%) in CHX, yielding the title compound 2-fluoro-6-methoxy-4-(4,4,5,5-tetramethyl-1,3,2-dioxaborolan-2-yl)benzaldehyde, E24-2, as an oil (1.230 g).

Method LCMS_JL5: Rt=1.12 min; [M+H]⁺=281.

### Step 2: 2-fluoro-6-methoxy-4-(2-methyl-1-oxo-1,2-dihydro-2,7-naphthyridin-4-yl)benzaldehyde (E24-3)

To a mixture of 4-bromo-2-methyl-2,7-naphthyridin-1(2H)-one, (Intermediate 5, 1.11 g, 1.393 mmol), 2-fluoro-6-methoxy-4-(4,4,5,5-tetramethyl-1,3,2-dioxaborolan-2-yl)benzaldehyde (E24-2, 0.7 g, 2.499 mmol) and Na₂CO₃ (0.6 g, 5.660 mmol) in a mixture of 1,4-dioxane (14 ml) and water (3.5 ml) under an argon atmosphere was added PdCl₂(dppf)-CH₂Cl₂ (0.1 g, 0.122 mmol) and the RM was stirred at 105° C. for 2.5 h. The mixture was cooled to RT, diluted with EtOAc and filtered over CELITE®. The solids were washed with EtOAc, the combined filtrates were concentrated, the residue was adsorbed on silica gel and purified by reverse phase chromatography on a REDISEP® Gold HP C18 column (50 g) eluting with ACN (from 4.8% to 100%) in an aq. solution of HCOOH (0.1%), yielding the title compound 2-fluoro-6-methoxy-4-(2-methyl-1-oxo-1,2-dihydro-2,7-naphthyridin-4-yl)benzaldehyde, E24-3, as a solid (152 mg).

Method LCMS_JL5: Rt=0.53 min; [M+H]⁺=313.

### Step 3: tert-butyl 4-((1-(2-fluoro-6-methoxy-4-(2-methyl-1-oxo-1,2-dihydro-2,7-naphthyridin-4-yl)benzyl)piperidin-4-yl)oxy)piperidine-1-carboxylate (E24-4)

To a solution of 2-fluoro-6-methoxy-4-(2-methyl-1-oxo-1,2-dihydro-2,7-naphthyridin-4-yl)benzaldehyde (E24-3, 152 mg, 0.487 mmol) in DCM (5 ml) were added tert-butyl 4-(piperidin-4-yloxy)piperidine-1-carboxylate (Intermediate 1, 208 mg, 0.730 mmol), NaOAc (43.9 mg, 0.535 mmol) and HOAc (28 μl, 0.487 mmol) and the RM was stirred RT for 5 min. Solid NaBH(OAc)₃ (206 mg, 0.973 mmol) was added and the RM was stirred at RT for 1 h. The mixture was added into a sat. aq. solution of NaHCO₃, diluted with DCM and water, the aqueous phase was extracted with DCM and the combined organic phases were washed with brine, dried over MgSO₄ and concentrated. The residue was adsorbed on silica gel and purified by chromatography on silica gel eluting with MeOH (from 0% to 20%) in DCM, yielding the title compound tert-butyl 4-((1-(2-fluoro-6-methoxy-4-(2-methyl-1-oxo-1,2-dihydro-2,7-naphthyridin-4-yl)benzyl)piperidin-4-yl)oxy)piperidine-1-carboxylate, E24-4, as a solid (143 mg).

Method LCMS_MLG-new-2: Rt=0.81 min; [M+H]⁺=581.

### Step 4: 1-((1-(2-(4-((1-(2-fluoro-6-methoxy-4-(2-methyl-1-oxo-1,2-dihydro-2,7-naphthyridin-4-yl)benzyl)piperidin-4-yl)oxy)piperidin-1-yl)ethyl)-2-oxo-1,2-dihydropyridin-3-yl)methyl)dihydropyrimidine-2,4(1H,3H)-dione (Compound E24)

To a solution of tert-butyl 4-((1-(2-fluoro-6-methoxy-4-(2-methyl-1-oxo-1,2-dihydro-2,7-naphthyridin-4-yl)benzyl)piperidin-4-yl)oxy)piperidine-1-carboxylate (E24-4, 143 mg, 0.246 mmol) in DCM (1 ml) was added TFA (1 ml, 12.98 mmol) and the RM was stirred at RT for 10 min. The mixture was concentrated and the residue was dissolved in DCM (2 ml) under an argon atmosphere at RT. TEA (220 μl, 1.587 mmol) was added, followed by DMF (1 ml), 2-(3-((2,4-dioxotetrahydropyrimidin-1(2H)-yl)methyl)-2-oxopyridin-1(2H)-yl)acetaldehyde (Intermediate 39, 86 mg, 0.327 mmol) and DCM (2 ml). Solid NaBH₃CN (104 mg, 0.492 mmol) was added and the RM was stirred at RT for 21 h. The mixture was quenched with a mixture of water, ACN and few drops of TFA, adsorbed on ISOLUTE® HM-N and purified by reverse phase chromatography on a REDISEP® Gold HP C18 column (15.5 g) eluting with ACN (from 1% to 100%) in an aq. solution of TFA (0.1%). Fractions containing the title compound were combined and the residue was purified by reverse phase chromatography on a REDISEP® Gold HP C18 column (15.5 g) eluting with ACN (from 1% to 100%) in an aq. solution of TFA (0.1%). Fractions containing the title compound were combined and filtered through a PL-HCO₃ MP SPE cartridge and the filtrate was concentrated. The residue was purified by SFC on a Princeton PPU column (250×30 mm, 100 Å, 5 m) eluting with MeOH (from 22% to 32%) in supercritical C02, followed by a purification by preparative HPLC on a Waters™ X-Bridge™ C18 column (100×30 mm, 5 μm) eluting with ACN (from 2% to 30%) in an aq. solution of TFA (0.1%). Fractions containing the title compound were combined, filtered through a PL-HCO₃ MP SPE cartridge and the combined filtrates were freeze-dried, yielding the title compound 1-((1-(2-(4-((1-(2-fluoro-6-methoxy-4-(2-methyl-1-oxo-1,2-dihydro-2,7-naphthyridin-4-yl)benzyl)piperidin-4-yl)oxy)piperidin-1-yl)ethyl)-2-oxo-1,2-dihydropyridin-3-yl)methyl)dihydropyrimidine-2,4(1H,3H)-dione, Compound E24, as a solid (12 mg).

Method LCMS_MLG3: Rt=0.88 min; [M+H]⁺=728.

¹H NMR (400 MHz, DMSO) δ [ppm] 10.14 (s, 1H), 9.44 (d, J=0.8 Hz, 1H), 8.73 (d, J=5.7 Hz, 1H), 7.91 (s, 1H), 7.62-7.49 (m, 2H), 7.26 (dd, J=6.8, 1.9 Hz, 1H), 7.01-6.87 (m, 2H), 6.19 (t, J=6.8 Hz, 1H), 4.26 (s, 2H), 3.98 (t, J=6.5 Hz, 2H), 3.85 (s, 3H), 3.59 (s, 3H), 3.56-3.52 (m, 2H), 3.44-3.35 (m, 4H), 2.76-2.68 (m, 4H), 2.58-2.52 (m, 4H), 2.22-2.03 (m, 4H), 1.80-1.67 (m, 4H), 1.43-1.27 (m, 4H).

### Compound E25: 1-(5-(2-(4-(((3S,4S)-4-(4-(2-Butyl-1-oxo-1,2-dihydro-2,7-naphthyridin-4-yl)-2-methoxyphenoxy)-3-fluoropiperidin-1-yl)methyl)piperidin-1-yl)-2-oxoethoxy)-2-methylphenyl)dihydropyrimidine-2,4(1H,3H)-dione

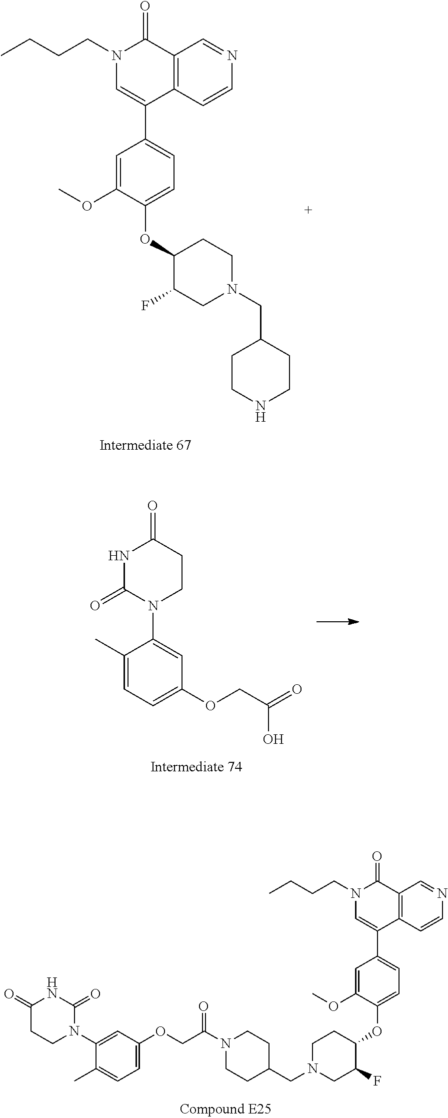 [SMILES: CCCCn1cc(-c2ccc(O[C@H]3CCN(CC4CCNCC4)C[C@@H]3F)c(OC)c2)c2ccncc2c1=O ; Cc1ccc(OCC(=O)O)cc1N1CCC(=O)NC1=O ; CCCCn1cc(-c2ccc(O[C@H]3CCN(CC4CCN(C(=O)COc5ccc(C)c(N6CCC(=O)NC6=O)c5)CC4)C[C@@H]3F)c(OC)c2)c2ccncc2c1=O]

To a solution of 2-(3-(2,4-dioxotetrahydropyrimidin-1(2H)-yl)-4-methylphenoxy)acetic acid (Intermediate 74, 26 mg, 0.09 mmol) and DIEA (0.050 ml, 0.286 mmol) in DMF (1 ml) was added HATU (39 mg, 0.103 mmol) and the RM was stirred at RT for 30 min. 2-Butyl-4-(4-(((3S,4S)-3-fluoro-1-(piperidin-4-ylmethyl)piperidin-4-yl)oxy)-3-methoxyphenyl)-2,7-naphthyridin-1(2H)-one TFA salt (Intermediate 67, 60 mg, 0.08 mmol) was added and the RM was stirred at RT for 24 h. The mixture was concentrated and the residue was purified by reverse phase chromatography on a REDISEP® C18 column (15.5 g) eluting with ACN (0% to 100%) in an aq. solution of TFA (0.1%). Fractions containing the title compound were combined, concentrated and the residue was purified by reverse phase chromatography on a REDISEP® C18 column (15.5 g) eluting with ACN (1% to 100%) in an aq. solution of NH₄HCO₃ (0.10%) yielding the title compound 1-(5-(2-(4-(((3S,4S)-4-(4-(2-Butyl-1-oxo-1,2-dihydro-2,7-naphthyridin-4-yl)-2-methoxyphenoxy)-3-fluoropiperidin-1-yl)methyl)piperidin-1-yl)-2-oxoethoxy)-2-methylphenyl)dihydropyrimidine-2,4(1H,3H)-dione, Compound E25, as a solid (26 mg).

Method LCMS_IJ1: Rt=0.72 min; [M+H]⁺=783.

¹H NMR (600 MHz, DMSO-d₆) δ[ppm] 10.33 (s, 1H), 9.43 (d, J=0.8 Hz, 1H), 8.71 (d, J=5.6 Hz, 1H), 7.77 (s, 1H), 7.49 (d, J=5.6 Hz, 1H), 7.20 (d, J=8.3 Hz, 1H), 7.16 (d, J=8.5 Hz, 1H), 7.07 (d, J=2.1 Hz, 1H), 6.95 (dd, J=8.2, 2.1 Hz, 1H), 6.89 (d, J=2.7 Hz, 1H), 6.81 (dd, J=8.5, 2.7 Hz, 1H), 4.82-4.59 (m, 3H), 4.40 (m, 1H), 4.31 (m, 1H), 4.03 (t, J=7.4 Hz, 2H), 3.82 (m, 5H), 3.52-3.46 (m, 1H), 3.29 (m, 3H), 3.06 (m, 2H), 2.81-2.64 (m, 3H), 2.23 (m, 2H), 2.10 (m, 5H), 1.83-1.57 (m, 6H), 1.34 (m, 2H), 1.12 (m, 1H), 0.92 (t, J=7.4 Hz, 3H).

### Compound E27: 1-((1-(2-(4-((1-(2,6-difluoro-4-(2-methyl-1-oxo-1,2-dihydro-2,7-naphthyridin-4-yl)phenethyl)piperidin-4-yl)oxy)piperidin-1-yl)ethyl)-2-oxo-1,2-dihydropyridin-3-yl)methyl)dihydropyrimidine-2,4(1H,3H)-dione

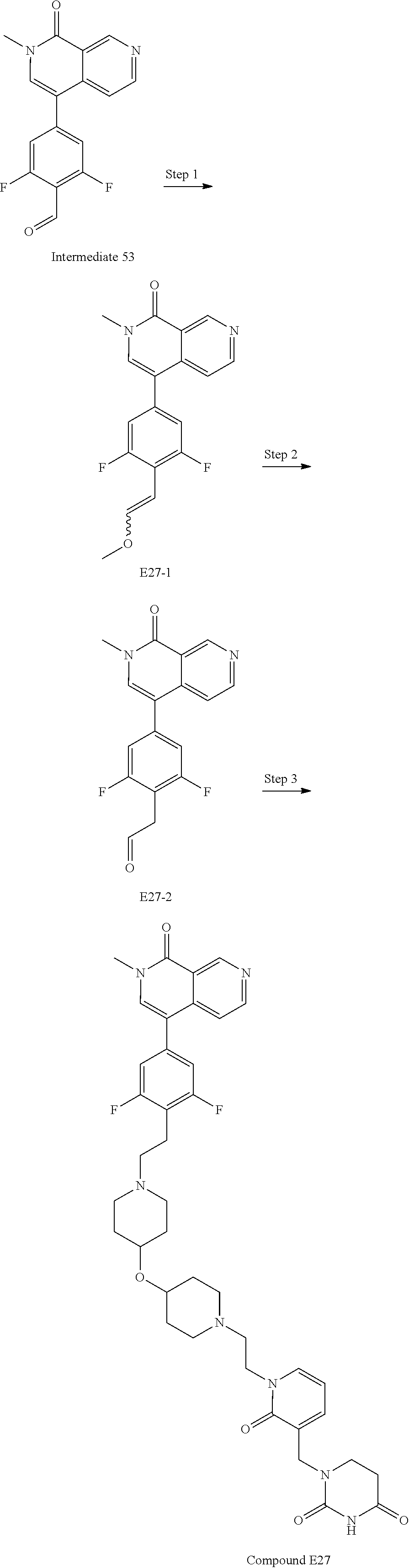 [SMILES: Cn1cc(-c2cc(F)c(C=O)c(F)c2)c2ccncc2c1=O ; COC=Cc1c(F)cc(-c2cn(C)c(=O)c3cnccc23)cc1F ; Cn1cc(-c2cc(F)c(CC=O)c(F)c2)c2ccncc2c1=O ; Cn1cc(-c2cc(F)c(CCN3CCC(OC4CCN(CCn5cccc(CN6CCC(=O)NC6=O)c5=O)CC4)CC3)c(F)c2)c2ccncc2c1=O]

### Step 1: (E,Z)-4-(3,5-difluoro-4-(2-methoxyvinyl)phenyl)-2-methyl-2,7-naphthyridin-1(2H)-one (E27-1)

To a solution of chloro(methoxymethyl)triphenylphosphorane (1.34 g, 3.90 mmol) in THE (5 ml) at 0° C. was added a solution of t-BuOK (437.25 mg, 3.90 mmol) in THF (5 ml) and the RM was stirred at 0° C. for 2 h. A solution of 2,6-difluoro-4-(2-methyl-1-oxo-1,2-dihydro-2,7-naphthyridin-4-yl)benzaldehyde (Intermediate 53, 900 mg) in THE (10 ml) was added and the RM was heated at 70° C. for 6 h. The mixture was concentrated, the residue was triturated in ACN (40 ml) and the mixture was filtered. The solids were purified by preparative HPLC on a Phenomenex Luna C18 column (150×40 mm, 15 μm) eluting with ACN (from 5% to 50%) in an aq. solution of TFA (0.1%), yielding a mixture of the title compounds (E,Z)-4-(3,5-difluoro-4-(2-methoxyvinyl)phenyl)-2-methyl-2,7-naphthyridin-1(2H)-one, E27-1, as an oil (200 mg).

Method LCMS WX3: Rt=0.94 min; [M+H]⁺=329.3.

### Step 2: 2-(2,6-difluoro-4-(2-methyl-1-oxo-1,2-dihydro-2,7-naphthyridin-4-yl)phenyl)acetaldehyde (E27-2)

To a solution of (E,Z)-4-(3,5-difluoro-4-(2-methoxyvinyl)phenyl)-2-methyl-2,7-naphthyridin-1(2H)-one (E27-1, 200 mg, 0.609 mmol) in acetone (10 ml) was added an aq. solution of HCl (2 M, 10 ml, 20 mmol) and the RM was stirred at 40° C. for 12 h. The mixture was concentrated, water (15 ml) was added and the aq. phase was extracted with DCM (5×20 ml). The combined organic phases were washed with brine (2×15 ml), dried over Na₂SO₄ and concentrated, yielding the title compound 2-(2,6-difluoro-4-(2-methyl-1-oxo-1,2-dihydro-2,7-naphthyridin-4-yl)phenyl)acetaldehyde, E27-2, as an oil (200 mg), which was used for the next step without further purification.

Method LCMS WX3: Rt=0.84 min; [M+Na]⁺=333.3.

### Step 3: 1-((1-(2-(4-((1-(2,6-difluoro-4-(2-methyl-1-oxo-1,2-dihydro-2,7-naphthyridin-4-yl)phenethyl)piperidin-4-yl)oxy)piperidin-1-yl)ethyl)-2-oxo-1,2-dihydropyridin-3-yl)methyl)dihydropyrimidine-2,4(1H,3H)-dione (Compound E27)

To a solution of 2-(2,6-difluoro-4-(2-methyl-1-oxo-1,2-dihydro-2,7-naphthyridin-4-yl)phenyl)acetaldehyde (E27-2, 200 mg) and 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione (Intermediate 40, 274.61 mg, 0.636 mmol) in a mixture of THF (2 ml) and EtOH (2 ml) was added DIEA (82.19 mg, 0.636 mmol) and a solution of ZnCl₂ (2 M) in THF (0.64 ml, 1.27 mmol) and the RM was stirred at 25° C. for 1 h. Solid NaBH₃CN (79.98 mg, 1.27 mmol) was added and the RM was stirred at 25° C. for 15 h. The mixture was concentrated, the residue was diluted with MeOH (15 ml), the mixture was filtered and the filtrate was concentrated. The residue was purified by preparative HPLC on a Phenomenex Luna C18 column (150×25 mm, 10 μm) eluting with ACN (from 5% to 50%) in an aq. solution of TFA (0.1%). Fractions containing the title compound were combined, concentrated, the pH of the aq. phase was adjusted to pH=9 by the addition of an aq. sat. solution of NaHCO₃ and the aq. phase was extracted with DCM (3×30 ml). The combined organic phases were washed with brine (2×15 ml), dried over Na₂SO₄ and concentrated, yielding the title compound 1-((1-(2-(4-((1-(2,6-difluoro-4-(2-methyl-1-oxo-1,2-dihydro-2,7-naphthyridin-4-yl)phenethyl)piperidin-4-yl)oxy)piperidin-1-yl)ethyl)-2-oxo-1,2-dihydropyridin-3-yl)methyl)dihydropyrimidine-2,4(1H,3H)-dione, Compound E27, as a solid (24.83 mg).

Method LCMS WX3: Rt=0.87 min; [M+H]⁺=730.7.

¹H NMR (400 MHz, DMSO-d₆) δ[ppm] 10.15 (s, 1H), 9.44 (s, 1H), 8.74 (d, J=5.6 Hz, 1H), 7.92 (s, 1H), 7.58 (dd, J=1.2, 6.8 Hz, 1H), 7.49 (d, J=5.6 Hz, 1H), 7.27 (br d, J=7.2 Hz, 1H), 7.22 (d, J=8.0 Hz, 2H), 6.20 (t, J=6.8 Hz, 1H), 4.26 (s, 2H), 3.99 (br t, J=6.4 Hz, 2H), 3.58 (s, 3H), 3.44-3.40 (m, 2H), 3.30 (s, 4H), 2.87-2.81 (m, 4H), 2.80-2.69 (m, 4H), 2.60-2.56 (m, 2H), 2.18-2.09 (m, 4H), 1.80-1.71 (m, 4H), 1.43-1.33 (m, 4H).

### Compound E28: 1-((1-(2-(4-(((3S,4S)-4-(4-(2-butyl-1-oxo-1,2-dihydro-2,7-naphthyridin-4-yl)phenoxy)-3-fluoropiperidin-1-yl)methyl)piperidin-1-yl)ethyl)-2-oxo-1,2-dihydropyridin-3-yl)methyl)dihydropyrimidine-2,4(1H,3H)-dione

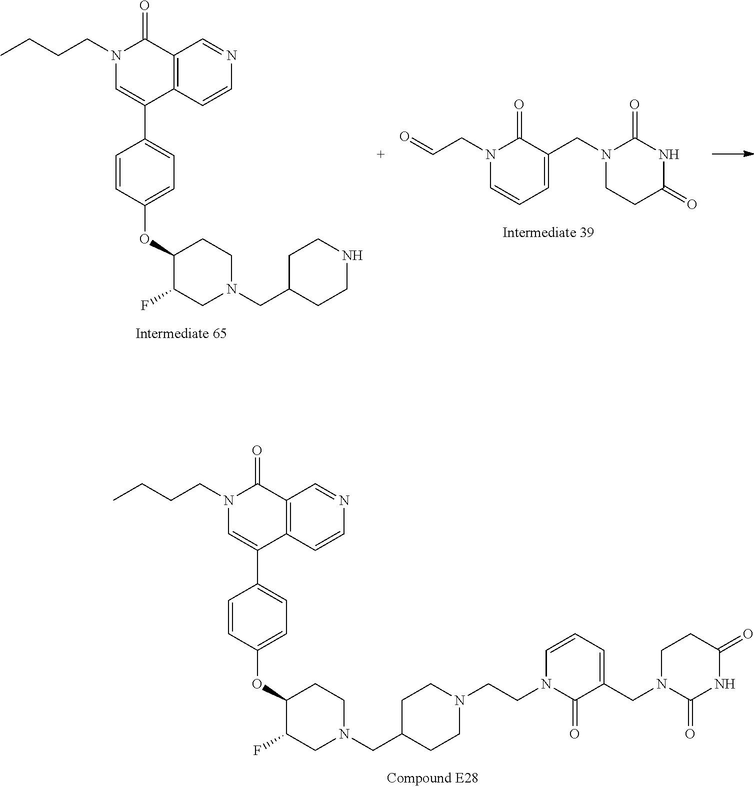 [SMILES: CCCCn1cc(-c2ccc(O[C@H]3CCN(CC4CCNCC4)C[C@@H]3F)cc2)c2ccncc2c1=O ; O=CCn1cccc(CN2CCC(=O)NC2=O)c1=O ; CCCCn1cc(-c2ccc(O[C@H]3CCN(CC4CCN(CCn5cccc(CN6CCC(=O)NC6=O)c5=O)CC4)C[C@@H]3F)cc2)c2ccncc2c1=O]

To a mixture of 2-butyl-4-(4-(((3S,4S)-3-fluoro-1-(piperidin-4-ylmethyl)piperidin-4-yl)oxy)phenyl)-2,7-naphthyridin-1(2H)-one TFA salt (Intermediate 65, 93 mg, 0.126 mmol), 2-(3-((2,4-dioxotetrahydropyrimidin-1(2H)-yl)methyl)-2-oxopyridin-1(2H)-yl)acetaldehyde (Intermediate 39, 48 mg, 0.182 mmol) and TEA (0.070 ml, 0.502 mmol) in a mixture of MeOH (1 ml) and DCM (1 ml) under an argon atmosphere, was added a solution of ZnCl₂ (0.5 M) in THE (0.350 ml, 0.175 mmol) and the RM was stirred at RT for 18 h. Solid NaBH₃CN (15 mg, 0.239 mmol) was added and the RM was stirred at RT for 24 h. The mixture was concentrated and the residue was purified by reverse phase chromatography on a REDISEP® Gold HP C18 column (50 g) eluting with ACN (from 0% to 100%) in an aq. solution of TFA (0.1%). Fractions containing the title compound were combined, concentrated and the residue was purified by reverse phase chromatography on a REDISEP® Gold HP C18 column (15.5 g) eluting with ACN (from 1% to 100%) in an aq. solution of NH₄HCO₃ (0.1%), yielding the title compound 1-((1-(2-(4-(((3S,4S)-4-(4-(2-butyl-1-oxo-1,2-dihydro-2,7-naphthyridin-4-yl)phenoxy)-3-fluoropiperidin-1-yl)methyl)piperidin-1-yl)ethyl)-2-oxo-1,2-dihydropyridin-3-yl)methyl)dihydropyrimidine-2,4(1H,3H)-dione, Compound E28, as a solid (44 mg).

Method LCMS_MLG3: Rt=0.98 min; [M+H]⁺=740.

¹H NMR (600 MHz, chloroform-d₃) δ 9.69 (s, 1H), 8.68 (d, J=5.6 Hz, 1H), 7.48 (m, 1H), 7.37 (d, J=5.6 Hz, 1H), 7.31-7.27 (m, 2H), 7.24 (m, 1H), 7.20 (m, 1H), 7.10-7.04 (m, 2H), 6.19 (m, 1H), 4.69 (m, 1H), 4.44 (s, 2H), 4.34 (m, 1H), 4.04 (m, 4H), 3.69 (t, J=6.9 Hz, 2H), 3.09 (m, 1H), 2.92 (m, 2H), 2.66 (m, 4H), 2.38-2.03 (m, 8H), 1.80 (m, 5H), 1.42 (m, 2H), 1.25 (m, 3H), 0.98 (t, J=7.4 Hz, 3H).

### Compound E29:1-(5-(4-(((3R,4R)-4-(4-(2-butyl-1-oxo-1,2-dihydro-2,7-naphthyridin-4-yl)-2-fluorophenoxy)-3-fluoropiperidin-1-yl)methyl)piperidine-1-carbonyl)-2-methoxyphenyl)dihydropyrimidine-2,4(1H,3H)-dione

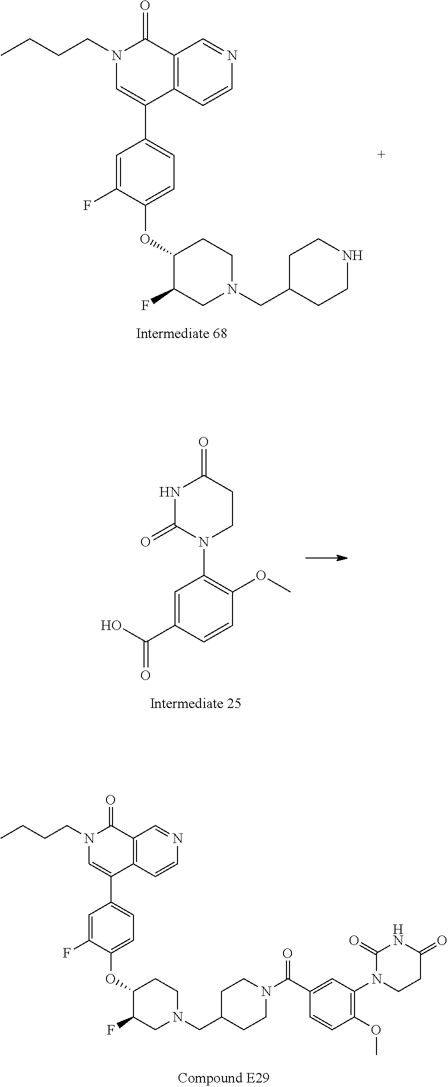 [SMILES: CCCCn1cc(-c2ccc(O[C@@H]3CCN(CC4CCNCC4)C[C@H]3F)c(F)c2)c2ccncc2c1=O ; COc1ccc(C(=O)O)cc1N1CCC(=O)NC1=O ; CCCCn1cc(-c2ccc(O[C@@H]3CCN(CC4CCN(C(=O)c5ccc(OC)c(N6CCC(=O)NC6=O)c5)CC4)C[C@H]3F)c(F)c2)c2ccncc2c1=O]

To a solution of 3-(2,4-dioxotetrahydropyrimidin-1(2H)-yl)-4-methoxybenzoic acid (Intermediate 25, 39 mg, 0.148 mmol) in DMF (0.5 ml) were added DIEA (0.050 ml, 0.286 mmol) and HATU (60 mg, 0.158 mmol) and the RM was stirred at RT for 30 min. A solution of 2-butyl-4-(3-fluoro-4-(((3R,4R)-3-fluoro-1-(piperidin-4-ylmethyl)piperidin-4-yl)oxy)phenyl)-2,7-naphthyridin-1(2H)-one TFA salt (Intermediate 68, 158 mg, 0.124 mmol) and DIEA (0.050 ml, 0.286 mmol) in DMF (0.5 ml) was added and the RM was stirred at RT for 2 h. The mixture was purified by reverse phase chromatography on a REDISEP® C18 column (15.5 g) eluting with ACN (1% to 100%) in an aq. solution of TFA (0.1%). Fractions containing the title compound were combined and the residue was purified by reverse phase chromatography on a REDISEP® C18 column (15.5 g) eluting with ACN (1% to 100%) in an aq. solution of NH₄HCO₃ (0.1%), yielding the title compound 1-(5-(4-(((3R,4R)-4-(4-(2-butyl-1-oxo-1,2-dihydro-2,7-naphthyridin-4-yl)-2-fluorophenoxy)-3-fluoropiperidin-1-yl)methyl)piperidine-1-carbonyl)-2-methoxyphenyl)dihydropyrimidine-2,4(1H,3H)-dione, Compound E29, as a solid (78 mg).

Method LCMS_MLG9: Rt=0.63 min; [M+H]⁺=757.

¹H NMR (400 MHz, DMSO-d₆) δ[ppm] 10.33 (s, 1H), 9.44 (s, 1H), 8.72 (d, J=5.6 Hz, 1H), 7.81 (s, 1H), 7.47-7.34 (m, 4H), 7.32 (m, 1H), 7.24 (s, 1H), 7.16 (d, J=8.5 Hz, 1H), 4.58 (m, 3H), 4.03 (t, J=7.3 Hz, 2H), 3.85 (m, 4H), 3.60 (t, J=6.7 Hz, 2H), 3.24-3.04 (m, 2H), 3.03-2.52 (m, 6H), 2.37-2.10 (m, 4H), 1.92-1.53 (m, 6H), 1.33 (m, 2H), 1.09 (m, 1H), 0.92 (t, J=7.3 Hz, 3H).

### Compound E32: 1-(5-(4-(((3S,4S)-4-(4-(2-Butyl-1-oxo-1,2-dihydro-2,7-naphthyridin-4-yl)-2-methoxyphenoxy)-3-fluoropiperidin-1-yl)methyl)piperidine-1-carbonyl)-2-methoxyphenyl)dihydropyrimidine-2,4(1H,3H)-dione

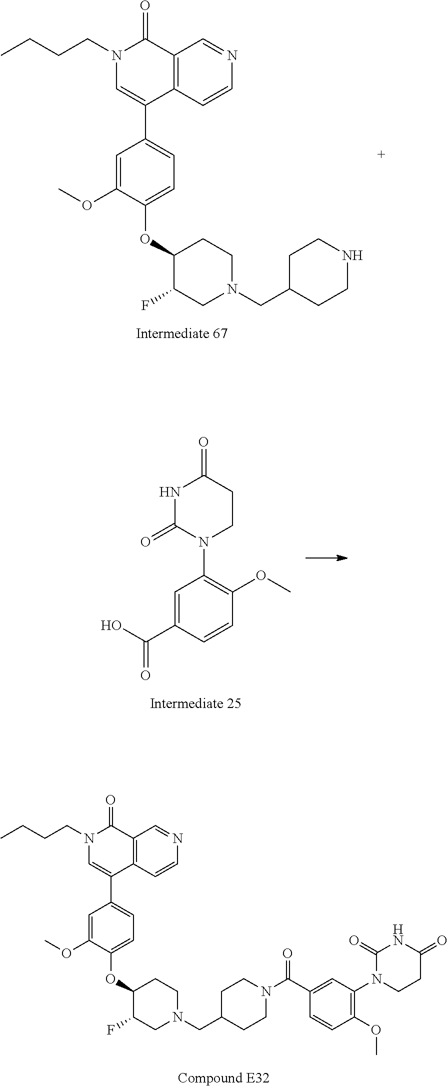 [SMILES: CCCCn1cc(-c2ccc(O[C@H]3CCN(CC4CCNCC4)C[C@@H]3F)c(OC)c2)c2ccncc2c1=O ; COc1ccc(C(=O)O)cc1N1CCC(=O)NC1=O ; CCCCn1cc(-c2ccc(O[C@H]3CCN(CC4CCN(C(=O)c5ccc(OC)c(N6CCC(=O)NC6=O)c5)CC4)C[C@@H]3F)c(OC)c2)c2ccncc2c1=O]

To a solution of 3-(2,4-dioxotetrahydropyrimidin-1(2H)-yl)-4-methoxybenzoic acid (Intermediate 25, 34 mg, 0.13 mmol) and DIEA (0.050 ml, 0.29 mmol) in DMF (0.5 ml) was added HATU (50 mg, 0.13 mmol) and the RM was stirred at RT for 30 min. A solution of 2-butyl-4-(4-(((3S,4S)-3-fluoro-1-(piperidin-4-ylmethyl)piperidin-4-yl)oxy)-3-methoxyphenyl)-2,7-naphthyridin-1(2H)-one TFA salt (Intermediate 67, 80.8 mg, 0.11 mmol) and DIEA (0.050 ml, 0.29 mmol) in DMF (0.5 ml) was added and the RM was stirred at RT for 2 h. The mixture was purified by reverse phase chromatography on a REDISEP® Gold HP C18 column (15.5 g) eluting with ACN (from 1% to 100%) in an aq. solution of NH₄HCO₃ (0.1%). Fractions containing the title compound were combined, concentrated and the residue was purified by SFC on a Waters Viridis 2-EP column (250×30 mm, 130 Å, 5 m) eluting with MeOH (from 16% to 24%) in supercritical CO₂, yielding the title compound 1-(5-(4-(((3S,4S)-4-(4-(2-butyl-1-oxo-1,2-dihydro-2,7-naphthyridin-4-yl)-2-methoxyphenoxy)-3-fluoropiperidin-1-yl)methyl)piperidine-1-carbonyl)-2-methoxyphenyl)dihydropyrimidine-2,4(1H,3H)-dione, Compound E32, as a solid (35 mg).

Method LCMS_MLG1: Rt=0.75 min; [M+H]⁺=769.

¹H NMR (400 MHz, DMSO-d₆) δ[ppm] 10.33 (s, 1H), 9.43 (s, 1H), 8.71 (d, J=5.6 Hz, 1H), 7.77 (s, 1H), 7.48 (d, J=5.7 Hz, 1H), 7.37 (dd, J=8.4, 2.2 Hz, 1H), 7.32 (d, J=2.1 Hz, 1H), 7.18 (dd, J=17.4, 8.4 Hz, 2H), 7.07 (d, J=2.0 Hz, 1H), 6.95 (dd, J=8.2, 2.1 Hz, 1H), 4.66 (m, 1H), 4.40 (m, 2H), 4.04 (m, 3H), 3.85 (s, 3H), 3.82 (s, 3H), 3.60 (t, J=6.7 Hz, 2H), 3.10 (m, 1H), 3.01-2.65 (m, 5H), 2.29-2.05 (m, 5H), 1.90-1.52 (m, 6H), 1.34 (m, 2H), 1.16-1.01 (m, 2H), 0.92 (t, J=7.4 Hz, 3H).

### Compound E34: 1-(5-(4-((4-(4-(2-butyl-1-oxo-1,2-dihydro-2,7-naphthyridin-4-yl)-2-fluorophenoxy)piperidin-1-yl)methyl)piperidine-1-carbonyl)-2-methoxyphenyl)dihydropyrimidine-2,4(1H,3H)-dione

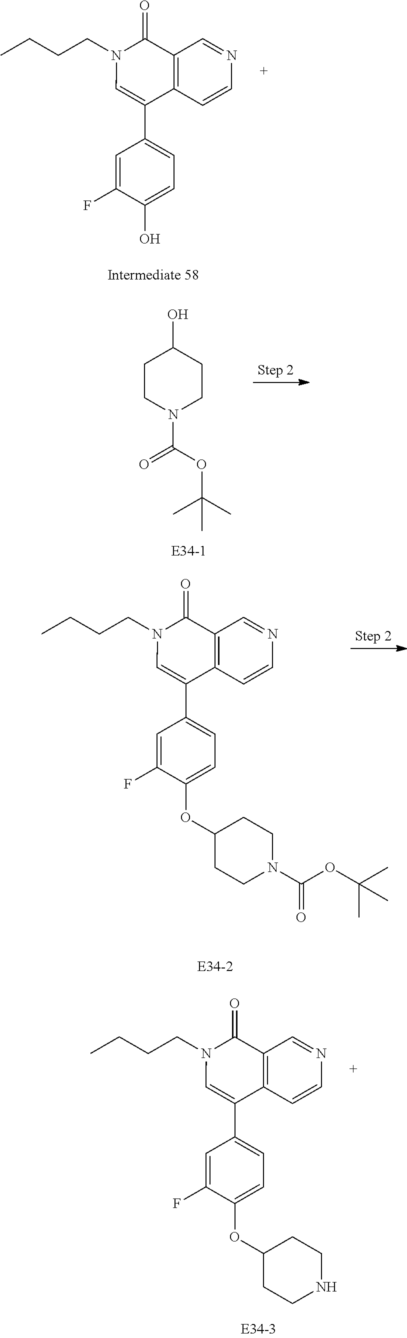

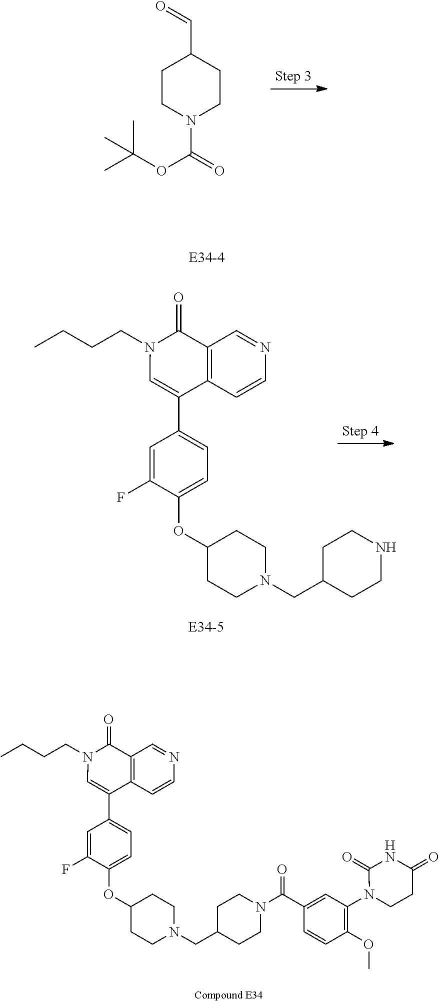 [SMILES: CC(C)(C)OC(=O)N1CCC(C=O)CC1 ; CCCCn1cc(-c2ccc(OC3CCN(CC4CCNCC4)CC3)c(F)c2)c2ccncc2c1=O ; CCCCn1cc(-c2ccc(OC3CCN(CC4CCN(C(=O)c5ccc(OC)c(N6CCC(=O)NC6=O)c5)CC4)CC3)c(F)c2)c2ccncc2c1=O]

### Step 1: tert-butyl 4-(4-(4,4,5,5-tetramethyl-1,3,2-dioxaborolan-2-yl)phenoxy)piperidine-1-carboxylate (E34-2)

To a solution of 2-butyl-4-(3-fluoro-4-hydroxyphenyl)-2,7-naphthyridin-1(2H)-one (Intermediate 58, 150 mg, 0.480 mmol), tert-butyl 4-hydroxypiperidine-1-carboxylate (E34-1, 108 mg, 0.526 mmol) and PPh₃ (139 mg, 0.530 mmol) in THE (4 ml) under an argon atmosphere was dropwise added DIAD (0.110 ml, 0.566 mmol) at RT and the RM was stirred at RT for 20 h. The mixture was concentrated and the residue was dissolved in toluene (4 ml). Tert-butyl 4-hydroxypiperidine-1-carboxylate (E34-1, 108 mg, 0.528 mmol) and PPh₃ (139 mg, 0.530 mmol) were added under an argon atmosphere, followed by DEAD (0.090 ml, 0.568 mmol) and the RM was stirred at RT for 20 h. The mixture was added into a mixture of EtOAc and an aq. sat. solution of NaHCO₃, the organic phase was washed with brine, dried over MgSO₄ and concentrated. The residue was purified by chromatography on silica gel eluting with EtOAc (from 0% to 100%) in CHX, yielding the title compound tert-butyl 4-(4-(2-butyl-1-oxo-1,2-dihydro-2,7-naphthyridin-4-yl)-2-fluorophenoxy)piperidine-1-carboxylate, E34-2, as a solid (659 mg).

Method LCMS_MLG8: Rt=1.32 min; [M+H]⁺=496.

### Step 2: 2-butyl-4-(3-fluoro-4-(piperidin-4-yloxy)phenyl)-2,7-naphthyridin-1(2H)-one (E34-3)

To a solution of tert-butyl 4-(4-(2-butyl-1-oxo-1,2-dihydro-2,7-naphthyridin-4-yl)-2-fluorophenoxy)piperidine-1-carboxylate (E34-2, 639 mg, 0.093 mmol) in DCM (2 ml) was added TFA (0.200 ml, 2.60 mmol) and the RM was stirred at RT for 1 h. The mixture was concentrated and the residue was purified by reverse phase chromatography on a REDISEP® Gold HP C18 column (15.5 g) eluting with ACN (from 1% to 100%) in an aq. solution of TFA (0.1%), yielding the title compound 2-butyl-4-(3-fluoro-4-(piperidin-4-yloxy)phenyl)-2,7-naphthyridin-1(2H)-one, E34-3, as a TFA salt (28 mg).

Method LCMS_MLG8: Rt=0.61 min; [M+H]⁺=396.

### Step 3: 2-butyl-4-(3-fluoro-4-((1-(piperidin-4-ylmethyl)piperidin-4-yl)oxy)phenyl)-2,7-naphthyridin-1(2H)-one (E34-5)

To a mixture of 2-buty-4-(3-fluoro-4-(piperidin-4-yloxy)phenyl)-2,7-naphthyridin-1(2H)-one TFA salt (E34-3, 28 mg, 0.071 mmol), TEA (0.030 ml, 0.212 mmol) and tert-butyl 4-formylpiperidine-1-carboxylate (E34-4, 20 mg, 0.094 mmol) in MeOH (1 ml) was added a solution of ZnCl₂ (0.5 M) in THF (0.170 ml, 0.085 mmol) and the RM was stirred under an argon atmosphere at RT for 7 h. Solid NaBH₃CN (8 mg, 0.127 mmol) was added and the RM was stirred at RT for 18 h. The mixture was concentrated, a mixture of DCM (1 ml) and TFA (0.150 ml, 1.95 mmol) was added and the RM was stirred at RT for 1 h. The mixture was concentrated and the residue was purified by reverse phase chromatography on a REDISEP® Gold HP C18 column (15.5 g) eluting with ACN (from 1% to 100%) in an aq. solution of TFA (0.1%), yielding the title compound 2-butyl-4-(3-fluoro-4-((1-(piperidin-4-ylmethyl)piperidin-4-yl)oxy)phenyl)-2,7-naphthyridin-1(2H)-one, E34-5, as a TFA salt (20 mg).

Method LCMS_MLG8: Rt=0.47 min; [M+H]⁺=493.

### Step 5: 1-(5-(4-((4-(4-(2-butyl-1-oxo-1,2-dihydro-2,7-naphthyridin-4-yl)-2-fluorophenoxy)piperidin-1-yl)methyl)piperidine-1-carbonyl)-2-methoxyphenyl)dihydropyrimidine-2,4(1H,3H)-dione (Compound E34)

To a solution of 3-(2,4-dioxotetrahydropyrimidin-1(2H)-yl)-4-methoxybenzoic acid (Intermediate 25, 8 mg, 0.030 mmol) in DMF (0.5 ml) was added DIEA (0.010 ml, 0.057 mmol), and HATU (12 mg, 0.032 mmol) and the RM was stirred at RT for 30 min. A solution of 2-butyl-4-(3-fluoro-4-((1-(piperidin-4-ylmethyl)piperidin-4-yl)oxy)phenyl)-2,7-naphthyridin-1(2H)-one TFA salt (E34-5, 20 mg, 0.028 mmol) and DIEA (0.020 ml, 0.115 mmol) in DMF (0.5 ml) was added and the RM was stirred at RT for 2 h. The mixture was purified by reverse phase chromatography on a REDISEP® Gold HP C18 column (15.5 g) eluting with ACN (from 1% to 100%) in an aq. solution of NH₄HCO₃ (0.1%), yielding the title compound 1-(5-(4-((4-(4-(2-butyl-1-oxo-1,2-dihydro-2,7-naphthyridin-4-yl)-2-fluorophenoxy)piperidin-1-yl)methyl)piperidine-1-carbonyl)-2-methoxyphenyl)dihydropyrimidine-2,4(1H,3H)-dione, Compound E34, as a solid (16 mg).

Method LCMS_MLG2: Rt=0.6 min; [M+H]⁺=739.

¹H NMR (400 MHz, DMSO-d₆) δ[ppm] 10.33 (s, 1H), 9.43 (s, 1H), 8.72 (d, J=5.7 Hz, 1H), 7.80 (s, 1H), 7.44 (d, J=5.7 Hz, 1H), 7.42-7.29 (m, 4H), 7.21 (d, J=8.4 Hz, 1H), 7.16 (d, J=8.6 Hz, 1H), 4.48 (m, 2H), 4.02 (t, J=7.3 Hz, 2H), 3.85 (s, 3H), 3.60 (t, J=6.6 Hz, 2H), 3.01 (m, 3H), 2.73-2.62 (m, 4H), 2.19 (m, 4H), 1.98 (m, 2H), 1.70 (m, 7H), 1.33 (m, 2H), 1.16-1.01 (m, 2H), 0.92 (t, J=7.4 Hz, 3H).

### Compound E35: 1-((1-(2-(4-(((cis)-4-(4-(2-butyl-1-oxo-1,2-dihydro-2,7-naphthyridin-4-yl)phenoxy)cyclohexyl)oxy)piperidin-1-yl)ethyl)-2-oxo-1,2-dihydropyridin-3-yl)methyl)dihydropyrimidine-2,4(1H,3H)-dione

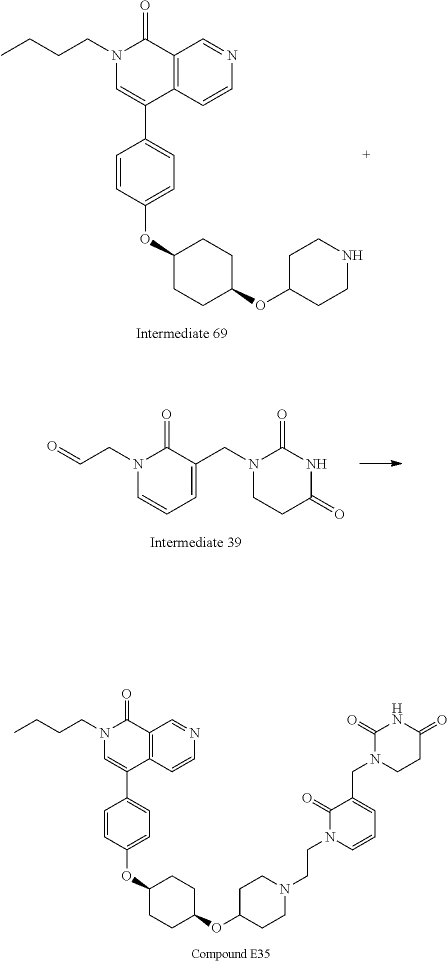 [SMILES: CCCCn1cc(-c2ccc(O[C@H]3CC[C@@H](OC4CCNCC4)CC3)cc2)c2ccncc2c1=O ; O=CCn1cccc(CN2CCC(=O)NC2=O)c1=O ; CCCCn1cc(-c2ccc(O[C@H]3CC[C@@H](OC4CCN(CCn5cccc(CN6CCC(=O)NC6=O)c5=O)CC4)CC3)cc2)c2ccncc2c1=O]

To a mixture of 2-butyl-4-(4-(((cis)-4-(piperidin-4-yloxy)cyclohexyl)oxy)phenyl)-2,7-naphthyridin-1(2H)-one (Intermediate 69, 135 mg, 0.233 mmol), 2-(3-((2,4-dioxotetrahydropyrimidin-1(2H)-yl)methyl)-2-oxopyridin-1(2H)-yl)acetaldehyde (Intermediate 39, 74 mg, 0.281 mmol) and TEA (0.080 ml, 0.574 mmol) in a mixture of MeOH (1 ml) and DCM (1 ml) under an argon atmosphere, was added a solution of ZnCl₂ (0.5 M) in THE (0.500 ml, 0.250 mmol) and the RM was stirred at RT for 18 h. Solid NaBH₃CN (23 mg, 0.366 mmol) was added and the RM was stirred at RT for 24 h. The mixture was concentrated and purified by reverse phase chromatography on a REDISEP® Gold HP C18 column (50 g) eluting with ACN (from 0% to 100%) in an aq. solution of TFA (0.1%). Fractions containing the title compound were combined, concentrated and the residue was purified by reverse phase chromatography on a REDISEP® Gold HP C18 column (15.5 g) eluting with ACN (from 1% to 100%) in an aq. solution of NH₄HCO₃ (0.1%), yielding the title compound 1-((1-(2-(4-(((cis)-4-(4-(2-butyl-1-oxo-1,2-dihydro-2,7-naphthyridin-4-yl)phenoxy)cyclohexyl)oxy)piperidin-1-yl)ethyl)-2-oxo-1,2-dihydropyridin-3-yl)methyl)dihydropyrimidine-2,4(1H,3H)-dione, Compound E35, as a solid (55 mg).

Method LCMS_MLG4: Rt=3.40 min; [M+H]⁺=723.

Method LCMS_MLG3: Rt=1.00 min; [M+H]⁺=723.

¹H NMR (400 MHz, DMSO-d₆) δ[ppm] 10.18 (s, 1H), 9.45 (d, J=0.8 Hz, 1H), 8.73 (d, J=5.6 Hz, 1H), 7.76 (s, 1H), 7.64-7.58 (m, 1H), 7.44 (m, 1H), 7.41-7.35 (m, 2H), 7.30 (m, 1H), 7.13-7.05 (m, 2H), 6.23 (m, 1H), 4.52 (m, 1H), 4.29 (s, 2H), 4.11-3.95 (m, 4H), 3.58 (m, 1H), 3.44 (m, 3H), 2.76 (m, 2H), 2.59 (t, J=6.8 Hz, 2H), 2.16 (m, 2H), 1.93-1.55 (m, 14H), 1.36 (m, 4H), 0.94 (t, J=7.4 Hz, 3H).

### Compound E36: 1-(5-(4-(((3R,4R)-1-(4-(2-butyl-1-oxo-1,2-dihydro-2,7-naphthyridin-4-yl)-2,5-dimethoxybenzyl)-3-fluoropiperidin-4-yl)oxy)piperidine-1-carbonyl)-2-methoxyphenyl)dihydropyrimidine-2,4(1H,3H)-dione

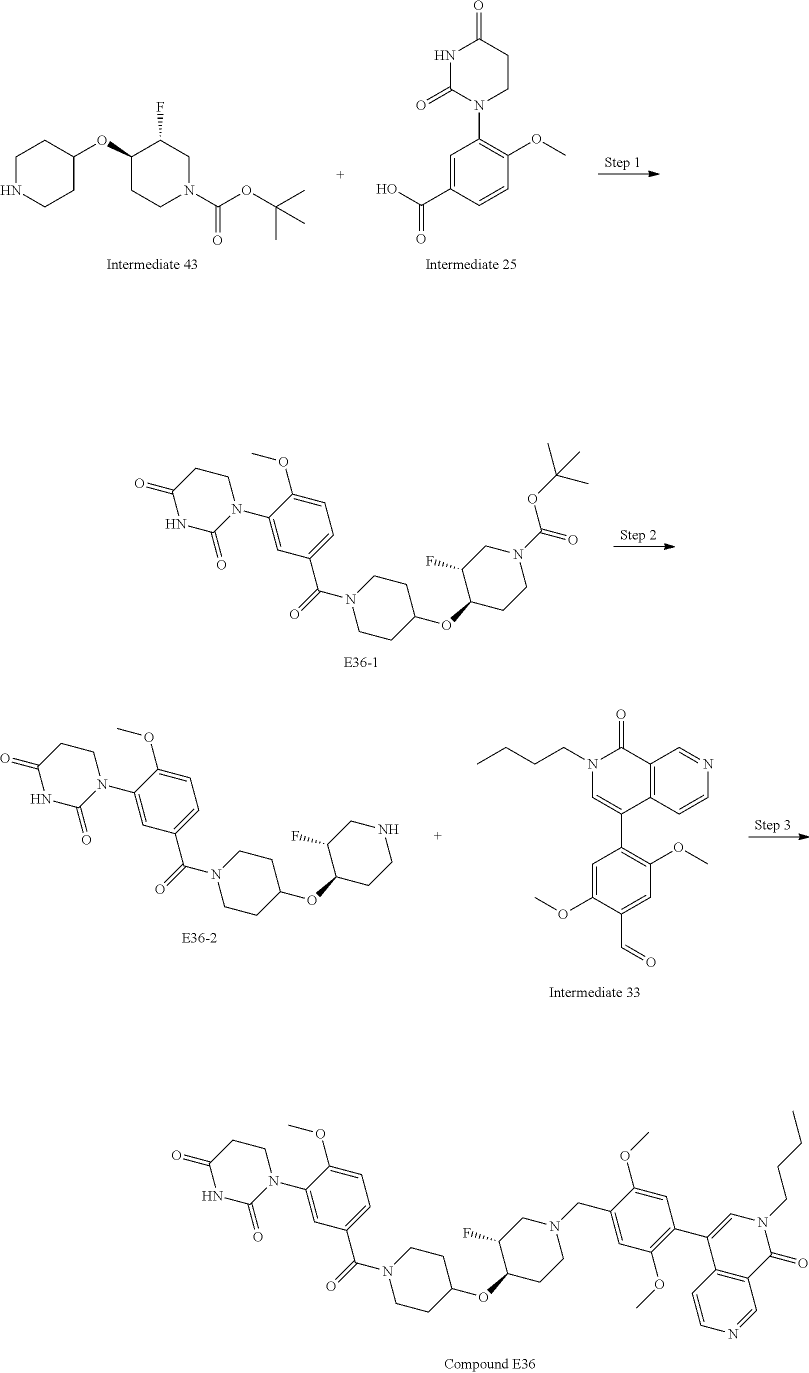 [SMILES: CC(C)(C)OC(=O)N1CC[C@@H](OC2CCNCC2)[C@H](F)C1 ; COc1ccc(C(=O)O)cc1N1CCC(=O)NC1=O ; COc1ccc(C(=O)N2CCC(O[C@@H]3CCN(C(=O)OC(C)(C)C)C[C@H]3F)CC2)cc1N1CCC(=O)NC1=O ; COc1ccc(C(=O)N2CCC(O[C@@H]3CCNC[C@H]3F)CC2)cc1N1CCC(=O)NC1=O ; CCCCn1cc(-c2cc(OC)c(C=O)cc2OC)c2ccncc2c1=O ; CCCCn1cc(-c2cc(OC)c(CN3CC[C@@H](OC4CCN(C(=O)c5ccc(OC)c(N6CCC(=O)NC6=O)c5)CC4)[C@H](F)C3)cc2OC)c2ccncc2c1=O]

### Step 1: tert-butyl (3R,4R)-4-((1-(3-(2,4-dioxotetrahydropyrimidin-1(2H)-yl)-4-methoxybenzoyl)piperidin-4-yl)oxy)-3-fluoropiperidine-1-carboxylate (E36-1)

To a solution of 3-(2,4-dioxotetrahydropyrimidin-1(2H)-yl)-4-methoxybenzoic acid (Intermediate 25, 116 mg, 0.440 mmol) in DMF (4 ml) was added DIEA (0.200 ml, 1.145 mmol), followed by HATU (183 mg, 0.480 mmol) and the RM was stirred for 30 min at RT. Tert-butyl (3R,4R)-3-fluoro-4-(piperidin-4-yloxy)piperidine-1-carboxylate 4-methylbenzenesulfonate salt (Intermediate 43, 200 mg, 0400 mmol) was added and the RM was stirred at RT for 4 h. The mixture was purified by reverse phase chromatography on a REDISEP® Gold HP C18 column (15.5 g) eluting with ACN (from 1% to 100%) in an aq. solution of TFA (0.1%), yielding the title compound tert-butyl (3R,4R)-4-((1-(3-(2,4-dioxotetrahydropyrimidin-1(2H)-yl)-4-methoxybenzoyl)piperidin-4-yl)oxy)-3-fluoropiperidine-1-carboxylate, E36-1, as a solid (185 mg).

Method LCMS_MLG1: Rt=0.94 min; [M+H]⁺=549.

### Step 2: 1-(5-(4-(((3R,4R)-3-fluoropiperidin-4-yl)oxy)piperidine-1-carbonyl)-2-methoxyphenyl)dihydropyrimidine-2,4(1H,3H)-dione (E36-2)

To a solution of tert-butyl (3R,4R)-4-((1-(3-(2,4-dioxotetrahydropyrimidin-1(2H)-yl)-4-methoxybenzoyl)piperidin-4-yl)oxy)-3-fluoropiperidine-1-carboxylate (E36-1, 180 mg, 0.295 mmol) in DCM (3 ml) was added TFA (0.7 ml, 9.09 mmol) and the RM was stirred at RT for 1 h. The mixture was concentrated, yielding the title compound 1-(5-(4-(((3R,4R)-3-fluoropiperidin-4-yl)oxy)piperidine-1-carbonyl)-2-methoxyphenyl)dihydropyrimidine-2,4(1H,3H)-dione, E36-2, as a solid TFA salt (175 mg), which was directly used for the next step without further purification.

Method LCMS_MLG3: Rt=0.53 min; [M+H]⁺=449.

### Step 3: 1-(5-(4-(((3R,4R)-1-(4-(2-butyl-1-oxo-1,2-dihydro-2,7-naphthyridin-4-yl)-2,5-dimethoxybenzyl)-3-fluoropiperidin-4-yl)oxy)piperidine-1-carbonyl)-2-methoxyphenyl)dihydropyrimidine-2,4(1H,3H)-dione (Compound E36)

To a mixture of 4-(2-butyl-1-oxo-1,2-dihydro-2,7-naphthyridin-4-yl)-2,5-dimethoxybenzaldehyde (Intermediate 33, 136 mg, 0.349 mmol), TEA (0.150 ml, 1.076 mmol) and 1-(5-(4-(((3R,4R)-3-fluoropiperidin-4-yl)oxy)piperidine-1-carbonyl)-2-methoxyphenyl)dihydropyrimidine-2,4(1H,3H)-dione TFA salt (E36-2, 175 mg, 0.296 mmol) in MeOH (3.5 ml) under an argon atmosphere, was added a solution of ZnCl₂ (0.5 M) in THE (0.80 ml, 0.40 mmol) and the RM was stirred at RT for 4 h. Solid NaBH₃CN (43 mg, 0.684 mmol) was added and the RM was stirred at RT for 2 h. The mixture was concentrated and the residue was purified by reverse phase chromatography on a REDISEP® Gold HP C18 column (15.5 g) eluting with ACN (from 1% to 100%) in an aq. solution of TFA (0.1%). Fractions containing the title compound were filtered through a PL-HCO₃ MP SPE cartridge, the filtrates were combined and freeze-dried. The residue was purified by reverse phase chromatography on a REDISEP® Gold HP C18 column (15.5 g) eluting with ACN (from 1% to 100%) in an aq. solution of NH₄HCO₃ (0.1%), yielding the title compound 1-(5-(4-(((3R,4R)-1-(4-(2-butyl-1-oxo-1,2-dihydro-2,7-naphthyridin-4-yl)-2,5-dimethoxybenzyl)-3-fluoropiperidin-4-yl)oxy)piperidine-1-carbonyl)-2-methoxyphenyl)dihydropyrimidine-2,4(1H,3H)-dione, Compound E36, as a solid (158 mg).

Method LCMS_MLG8: Rt=0.58 min; [M+H]⁺=799.

¹H NMR (600 MHz, DMSO-d₆) δ[ppm] 10.33 (s, 1H), 9.41 (s, 1H), 8.64 (d, J=5.6 Hz, 1H), 7.72 (s, 1H), 7.38 (dd, J=8.4, 2.1 Hz, 1H), 7.34 (d, J=2.1 Hz, 1H), 7.19-7.10 (m, 2H), 7.05 (d, J=5.6 Hz, 1H), 6.93 (s, 1H), 4.55-4.34 (m, 1H), 4.03 (m, 2H), 3.84 (s, 3H), 3.75 (m, 4H), 3.72-3.47 (m, 10H), 3.24 (m, 2H), 3.07 (m, 1H), 2.76 (m, 1H), 2.68 (t, J=6.7 Hz, 2H), 2.28-2.12 (m, 2H), 2.04-1.76 (m, 3H), 1.70 (m, 2H), 1.55-1.40 (m, 3H), 1.35 (m, 2H), 0.92 (t, J=7.3 Hz, 3H).

### Compound E37: 1-((1-(2-(4-((1-(4-(2-methyl-1-oxo-1,2-dihydro-2,7-naphthyridin-4-yl)benzyl)piperidin-4-yl)oxy)piperidin-1-yl)ethyl)-2-oxo-1,2-dihydropyridin-3-yl)methyl)dihydropyrimidine-2,4(1H,3H)-dione

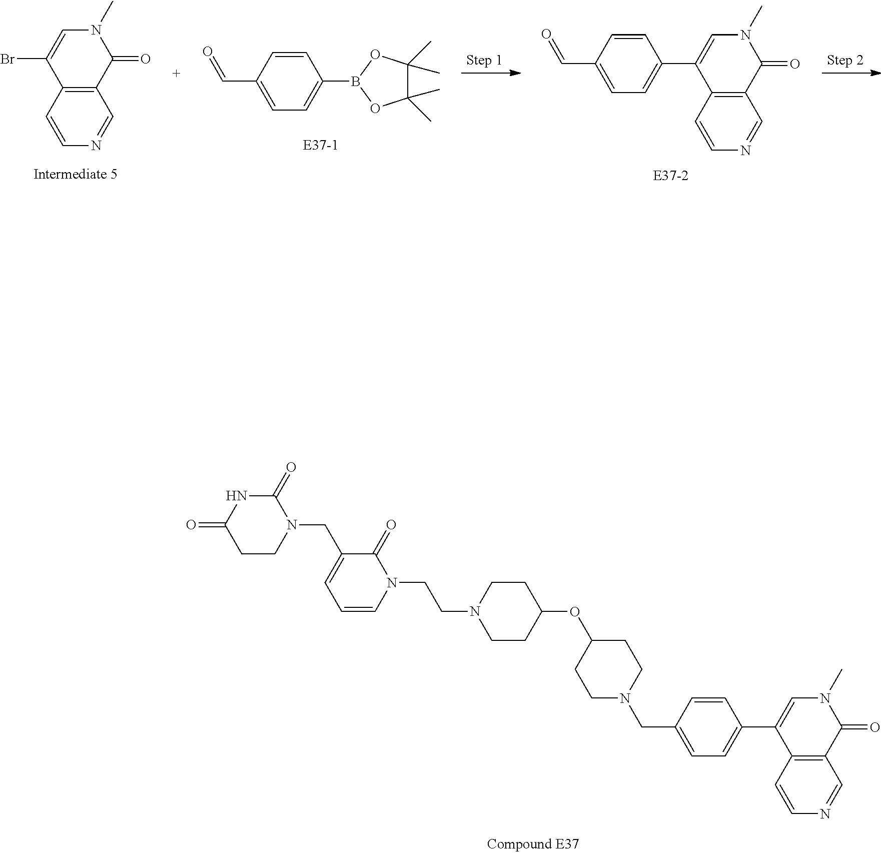 [SMILES: Cn1cc(Br)c2ccncc2c1=O ; CC1(C)OB(c2ccc(C=O)cc2)OC1(C)C ; Cn1cc(-c2ccc(C=O)cc2)c2ccncc2c1=O ; Cn1cc(-c2ccc(CN3CCC(OC4CCN(CCn5cccc(CN6CCC(=O)NC6=O)c5=O)CC4)CC3)cc2)c2ccncc2c1=O]

### Step 1: 4-(2-methyl-1-oxo-1,2-dihydro-2,7-naphthyridin-4-yl)benzaldehyde (E37-2)

To a solution of 4-bromo-2-methyl-2,7-naphthyridin-1(2H)-one (Intermediate 5, 3 g, 12.55 mmol) and 4-(4,4,5,5-tetramethyl-1,3,2-dioxaborolan-2-yl)benzaldehyde (E37-1, 2.07 g, 13.8 mmol) in a mixture of 1,4-dioxane (25 ml) and water (5 ml) under a nitrogen atmosphere was added Cs₂CO₃ (8.18 g, 25.10 mmol) and Pd(dppf)Cl₂ (918.19 mg, 1.25 mmol) at 25° C. The RM was stirred at 80° C. for 2 h. The mixture was filtered, the solids were triturated with EtOAc, filtered and dried, yielding the title compound 4-(2-methyl-1-oxo-1,2-dihydro-2,7-naphthyridin-4-yl)benzaldehyde, E37-2, as a solid, which was used for the next step without further purification.

Method LCMS WX2: Rt=0.56 min; [M+H]⁺=264.

### Step 2: 1-((1-(2-(4-((1-(4-(2-methyl-1-oxo-1,2-dihydro-2,7-naphthyridin-4-yl)benzyl)piperidin-4-yl)oxy)piperidin-1-yl)ethyl)-2-oxo-1,2-dihydropyridin-3-yl)methyl)dihydropyrimidine-2,4(1H,3H)-dione (Compound E37)

To a solution of 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione hydrochloride (Intermediate 40, 300 mg, 0.69 mmol) and 4-(2-methyl-1-oxo-1,2-dihydro-2,7-naphthyridin-4-yl)benzaldehyde (E37-2,183.73 mg, 0.69 mmol) in a mixture of THF (2 ml) and EtOH (2 ml) was added DIEA (134.78 mg, 1.39 mmol) and a solution of ZnCl₂ (1 M) in TH (1.39 ml, 1.39 mmol) and the RM was stirred at 25° C. for 2 h. Solid NaBH₃CN (86.97 mg, 1.38 mmol) was added and the RM was stirred at 25° C. for 10 h. The mixture was diluted with a sat. aq. solution of NaHCO₃(10 ml), the aq. phase was extracted with DCM (2×10 ml), the combined organic phases were washed with brine (10 ml), dried over Na₂SO₄ and concentrated. The residue was purified by preparative HPLC on a Phenomenex Luna C18 column (150×40 mm, 15 μm) eluting with ACN (from 5% to 45%) in an aq. solution of TFA (0.1%), followed by a purification by preparative HPLC on a Waters XBridge column (150×25 mm, 5 μm) eluting with ACN (from 5% to 50%) in an aq. solution of NH₄HCO₃ (0.05%), yielding the title compound 1-((1-(2-(4-((1-(4-(2-methyl-1-oxo-1,2-dihydro-2,7-naphthyridin-4-yl)benzyl)piperidin-4-yl)oxy)piperidin-1-yl)ethyl)-2-oxo-1,2-dihydropyridin-3-yl)methyl)dihydropyrimidine-2,4(1H,3H)-dione, Compound E37, as a solid (41.52 mg).

Method LCMS WX3: Rt=0.72 min; [M+H]⁺=680.

¹H NMR (400 MHz, DMSO-d₆) δ[ppm] 10.33 (s, 1H), 9.44 (d, J=0.7 Hz, 1H), 8.71 (d, J=5.6 Hz, 1H), 7.82 (s, 1H), 7.58 (dd, J=2.0, 6.6 Hz, 1H), 7.47-7.39 (m, 5H), 7.31-7.24 (m, 1H), 6.20 (t, J=6.8 Hz, 1H), 4.27 (s, 2H), 3.99 (t, J=6.4 Hz, 2H), 3.59 (s, 3H), 3.51 (s, 2H), 3.42 (m, J=6.8 Hz, 4H), 2.79-2.67 (m, 4H), 2.57 (t, J=6.8 Hz, 2H), 2.55-2.52 (m, 2H), 2.11 (m, J=10.0 Hz, 4H) 1.87-1.69 (m, 4H), 1.52-1.29 (m, 4H).

### Compound E39: 1-((1-(2-(4-(((3R,4R)-1-(2,5-Dimethoxy-4-(2-methyl-1-oxo-1,2-dihydro-2,7-naphthyridin-4-yl)benzyl)-3-fluoropiperidin-4-yl)oxy)piperidin-1-yl)ethyl)-2-oxo-1,2-dihydropyridin-3-yl)methyl)dihydropyrimidine-2,4(1H,3H)-dione

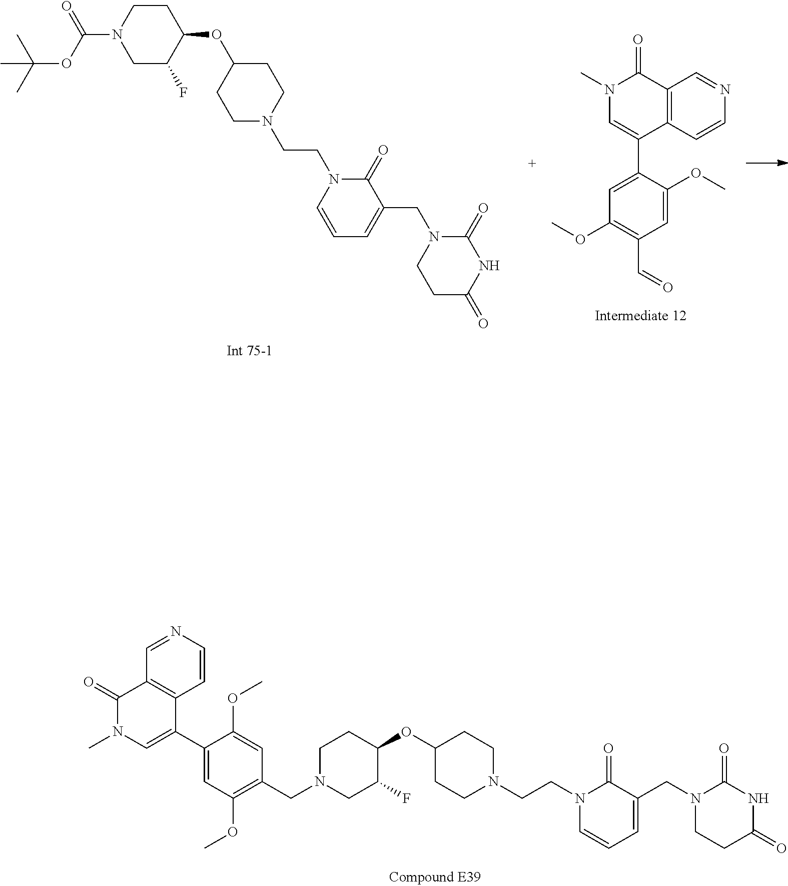

To a solution of tert-butyl (3R,4R)-4-((1-(2-(3-((2,4-dioxotetrahydropyrimidin-1(2H)-yl)methyl)-2-oxopyridin-1(2H)-yl)ethyl)piperidin-4-yl)oxy)-3-fluoropiperidine-1-carboxylate (Int 75-1, 155 mg, 0.282 mmol) in DCM (3 ml) under an argon atmosphere was added a solution of HCl (4 M) in 1,4-dioxane (1 ml, 4.0 mmol) and the RM was stirred at RT for 1 h. The mixture was concentrated and the residue was suspended in a mixture of DCM (3 ml) and DMF (1 ml) at RT under an argon atmosphere. TEA (78 μl, 0.564 mmol), 2,5-dimethoxy-4-(2-methyl-1-oxo-1,2-dihydro-2,7-naphthyridin-4-yl)benzaldehyde (Intermediate 12, 0.1 g, 0.308 mmol) and NaBH(OAc)₃ (0.179 g, 0.846 mmol) were added and the RM was stirred at RT for 18 h. The mixture was concentrated, the residue was adsorbed on ISOLUTE® HM-N and purified by reverse phase chromatography on a REDISEP® Gold HP C18 column (50 g) eluting with ACN (from 1% to 100%) in an aq. solution of HCOOH (0.1%). Fractions containing the title compound were combined, concentrated and the residue was purified by reverse phase chromatography on a REDISEP® Gold HP C18 column (5.5 g) eluting with ACN (from 5% to 100%) in an aq. solution of HCOOH (0.1%). Fractions containing the title compound were combined, concentrated and the residue was purified by reverse phase chromatography on a REDISEP® Gold HP C18 column (5.5 g) eluting with ACN (from 1% to 100%) in an aq. solution of HCOOH (0.1%), yielding the title compound 1-((1-(2-(4-(((3R,4R)-1-(2,5-dimethoxy-4-(2-methyl-1-oxo-1,2-dihydro-2,7-naphthyridin-4-yl)benzyl)-3-fluoropiperidin-4-yl)oxy)piperidin-1-yl)ethyl)-2-oxo-1,2-dihydropyridin-3-yl)methyl)dihydropyrimidine-2,4(1H,3H)-dione, Compound E39, as a solid HCOOH salt (8 mg).

Method LCMS_JL1: Rt=3.32 min; [M+H]⁺=758.6.

¹H NMR (400 MHz, DMSO-d₆) δ[ppm] 10.14 (s, 1H), 9.40 (s, 1H), 8.64 (d, J=5.6 Hz, 1H), 8.14 (s, 1H, HCOOH), 7.74 (s, 1H), 7.58 (dd, J=6.8, 2.0 Hz, 1H), 7.27 (dd, J=6.9, 1.9 Hz, 1H), 7.12 (s, 1H), 7.03 (d, J=5.6 Hz, 1H), 6.93 (s, 1H), 6.21 (t, J=6.8 Hz, 1H), 4.53-4.32 (m, 1H), 4.26 (s, 2H), 4.00 (t, J=6.4 Hz, 2H), 3.75 (s, 3H), 3.63 (s, 3H), 3.62-3.59 (m, 2H), 3.57 (s, 3H), 3.51-3.47 (m, 2H), 3.41 (t, J=6.8 Hz, 3H), 3.08-3.00 (m, 1H), 2.81-2.69 (m, 3H), 2.59-2.54 (m, 3H), 2.28-2.09 (m, 4H), 2.00-1.88 (m, 1H), 1.84-1.74 (m, 2H), 1.54-1.33 (m, 3H).

### Compound E40: 1-(5-(4-((1-(4-(2-Hexyl-1-oxo-1,2-dihydro-2,7-naphthyridin-4-yl)-2,6-dimethoxybenzyl)piperidin-4-yl)oxy)piperidine-1-carbonyl)-2-methoxyphenyl)dihydropyrimidine-2,4(1H,3H)-dione

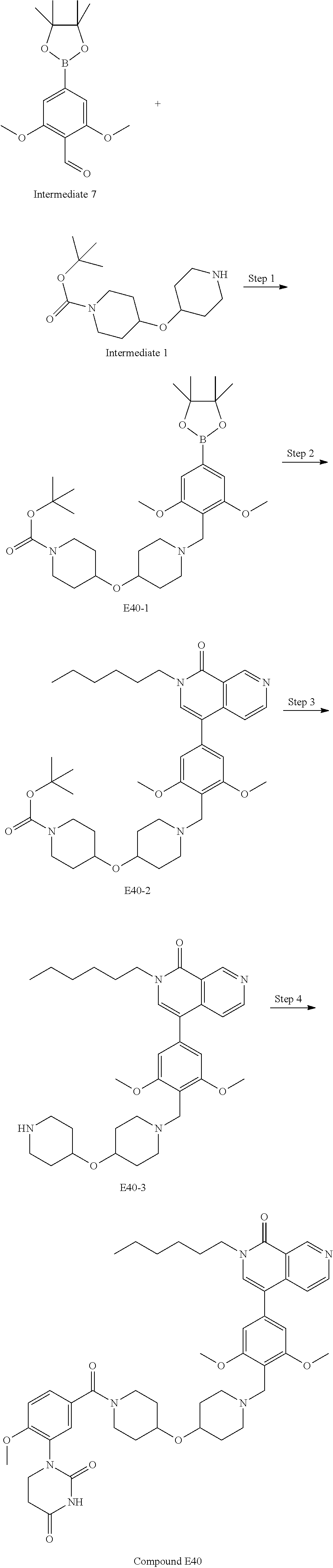

### Step 1: tert-butyl 4-((1-(2,6-dimethoxy-4-(4,4,5,5-tetramethyl-1,3,2-dioxaborolan-2-yl)benzyl)piperidin-4-yl)oxy)piperidine-1-carboxylate (E40-1)

To a solution of 2,6-dimethoxy-4-(4,4,5,5-tetramethyl-1,3,2-dioxaborolan-2-yl)benzaldehyde (Intermediate 7, 400 mg, 1.37 mmol) and tert-butyl 4-(piperidin-4-yloxy)piperidine-1-carboxylate (Intermediate 1, 500 mg, 1.76 mmol) in DMSO (4 ml) under an argon atmosphere was added a solution of ZnCl₂ (0.5 M) in THE (4 ml, 2.0 mmol) and the RM was stirred at RT for 4 h. Solid NaBH₃CN (184 mg, 2.93 mmol) was added and the RM was stirred at 50° C. overnight. The mixture was diluted with water, the aq. phase was extracted with EtOAc, the organic phase was washed with brine, dried over MgSO₄ and concentrated, yielding the title compound tert-butyl 4-((1-(2,6-dimethoxy-4-(4,4,5,5-tetramethyl-1,3,2-dioxaborolan-2-yl)benzyl)piperidin-4-yl)oxy)piperidine-1-carboxylate, E40-1 (832 mg), which was used for the next step without further purification.

LCMS analysis showed peaks corresponding to the title compound boronic ester and its corresponding boronic acid derivative.

Method LCMS_MLG1: Rt=1.04 min; [M+H]⁺=561 and Rt=0.86 min; [M+H]⁺=479.

### Step 2: tert-butyl 4-((1-(4-(2-hexyl-1-oxo-1,2-dihydro-2,7-naphthyridin-4-yl)-2,6-dimethoxybenzyl)piperidin-4-yl)oxy)piperidine-1-carboxylate (E40-2)

To a mixture of 4-bromo-2-hexyl-2,7-naphthyridin-1(2H)-one (Intermediate 48, 70 mg, 0.215 mmol), K₂CO₃ (89 mg, 0.645 mmol) and tert-butyl 4-((1-(2,6-dimethoxy-4-(4,4,5,5-tetramethyl-1,3,2-dioxaborolan-2-yl)benzyl)piperidin-4-yl)oxy)piperidine-1-carboxylate (E40-1, 200 mg, 0.253 mmol) in a mixture of ACN (2 ml) and water (0.5 ml) under an argon atmosphere was added Pd(dppf)Cl₂ (16 mg, 0.022 mmol) and the RM was heated at 100° C. for 1 h. The mixture was cooled to RT, filtered over CELITE® and the filtrate was concentrated. The residue was purified by reverse phase chromatography on a REDISEP® C18 column (15.5 g) eluting with ACN (2% to 100%) in an aq. solution of TFA (0.1%), yielding the title compound tert-butyl 4-((1-(4-(2-hexyl-1-oxo-1,2-dihydro-2,7-naphthyridin-4-yl)-2,6-dimethoxybenzyl)piperidin-4-yl)oxy)piperidine-1-carboxylate, E40-2, as a solid TFA salt (47 mg).

Method LCMS_MLG1: Rt=1.02 min; [M+H]⁺=663.

### Step 3: 4-(3,5-dimethoxy-4-((4-(piperidin-4-yloxy)piperidin-1-yl)methyl)phenyl)-2-hexyl-2,7-naphthyridin-1(2H)-one (E40-3)

To a solution of tert-butyl 4-((1-(4-(2-hexyl-1-oxo-1,2-dihydro-2,7-naphthyridin-4-yl)-2,6-dimethoxybenzyl)piperidin-4-yl)oxy)piperidine-1-carboxylate (E40-2, 47 mg, 0.057 mmol) in DCM (2 ml) was added TFA (0.100 ml, 1.298 mmol) and the RM was stirred at RT for 1 h. The mixture was concentrated, yielding the title compound 4-(3,5-dimethoxy-4-((4-(piperidin-4-yloxy)piperidin-1-yl)methyl)phenyl)-2-hexyl-2,7-naphthyridin-1(2H)-one, E40-3, as a TFA salt (59.8 mg), which was used for the next step without further purification.

Method LCMS_MLG1: Rt=0.77 min; [M+H]⁺=563.

### Step 4: 1-(5-(4-((1-(4-(2-hexyl-1-oxo-1,2-dihydro-2,7-naphthyridin-4-yl)-2,6-dimethoxybenzyl)piperidin-4-yl)oxy)piperidine-1-carbonyl)-2-methoxyphenyl)dihydropyrimidine-2,4(1H,3H)-dione (Compound E40)

To a solution of 3-(2,4-dioxotetrahydropyrimidin-1(2H)-yl)-4-methoxybenzoic acid (Intermediate 25, 18 mg, 0.068 mmol) and DIEA (0.030 ml, 0.172 mmol) in DMF (0.5 ml) was added HATU (27 mg, 0.071 mmol) and the RM was stirred at RT for 30 min. A solution of 4-(3,5-dimethoxy-4-((4-(piperidin-4-yloxy)piperidin-1-yl)methyl)phenyl)-2-hexyl-2,7-naphthyridin-1(2H)-one TFA salt (E40-3, 59.8 mg, 0.057 mmol) and DIEA (0.030 ml, 0.172 mmol) in DMF (0.5 ml) was added and the RM was stirred at RT for three days. The mixture was purified by reverse phase chromatography on a REDISEP® C18 column (15.5 g) eluting with ACN (2% to 100%) in an aq. solution of TFA (0.1%), yielding the title compound 1-(5-(4-((1-(4-(2-hexyl-1-oxo-1,2-dihydro-2,7-naphthyridin-4-yl)-2,6-dimethoxybenzyl)piperidin-4-yl)oxy)piperidine-1-carbonyl)-2-methoxyphenyl)dihydropyrimidine-2,4(1H,3H)-dione, Compound E40, as a solid (19 mg).

Method LCMS_MLG1: Rt=0.87 min; [M+H]⁺=809.

¹H NMR (400 MHz, DMSO-d₆) δ[ppm] 10.32 (s, 1H), 9.44 (m, 1H), 8.72 (m, 1H), 7.85 (m, 1H), 7.57 (m, 1H), 7.45-7.26 (m, 2H), 7.15 (m, 1H), 6.71 (s, 2H), 4.17-3.44 (m, 19H), 3.19 (m, 2H), 2.71 (m, 4H), 2.15 (m, 2H), 1.74 (m, 6H), 1.34 (m, 10H), 0.86 (m, 3H).

### Compound E42: 1-(5-(4-((4-(4-(2-butyl-1-oxo-1,2-dihydro-2,7-naphthyridin-4-yl)phenoxy)piperidin-1-yl)methyl)piperidine-1-carbonyl)-2-methoxyphenyl)dihydropyrimidine-2,4(1H,3H)-dione

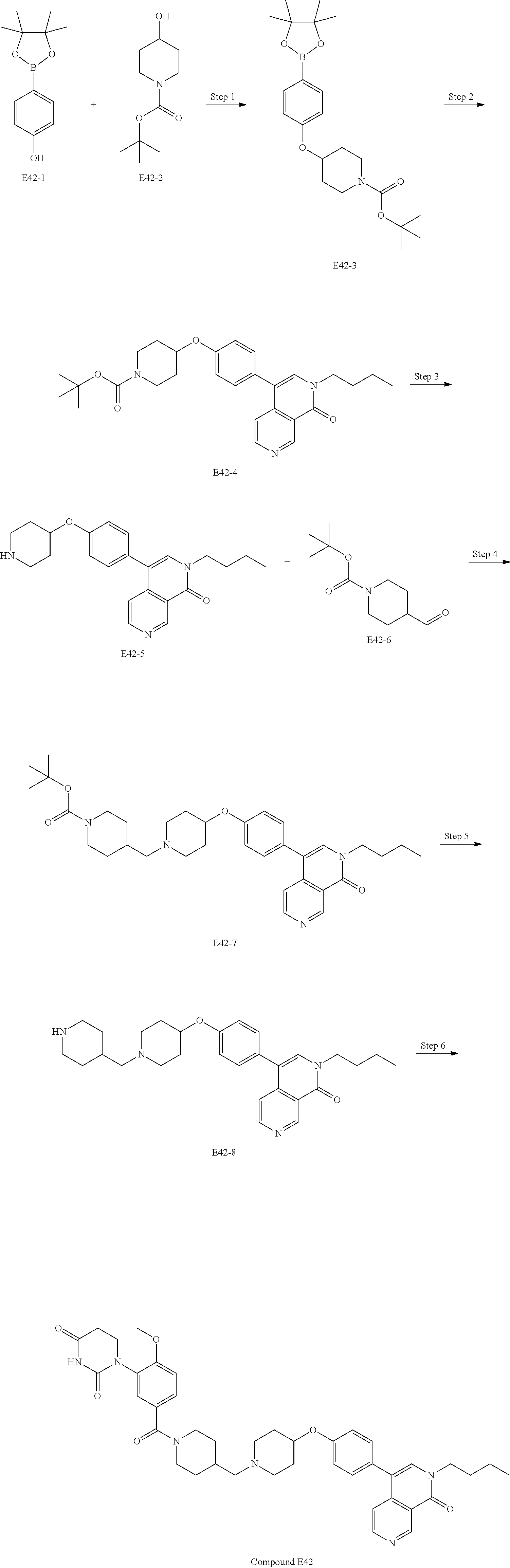

### Step 1: tert-butyl 4-(4-(4,4,5,5-tetramethyl-1,3,2-dioxaborolan-2-yl)phenoxy)piperidine-1-carboxylate (E42-3)

To a mixture of 4-(4,4,5,5-tetramethyl-1,3,2-dioxaborolan-2-yl)phenol (E42-1, 1.204 g, 5.47 mmol), tert-butyl 4-hydroxypiperidine-1-carboxylate (E42-2, 1.0 g, 4.97 mmol) and PPh₃ (1.955 g, 7.455 mmol) in THE (30 ml) was added DEAD (1.731 g, 9.94 mmol) at 0° C. and the RM was stirred at RT overnight. The mixture was diluted with EtOAc (30 ml), the organic phase was washed with water (30 ml) and brine (20 ml), dried over Na₂SO₄ and concentrated. The residue was purified by chromatography on silica gel eluting with EtOAc (from 0% to 20%) in PE, yielding the title compound tert-butyl 4-(4-(4,4,5,5-tetramethyl-1,3,2-dioxaborolan-2-yl)phenoxy)piperidine-1-carboxylate, E42-3, as a solid (650 mg).

Method LCMSA043: Rt=2.65 min; [M-Boc+H]⁺=348.

### Step 2: tert-butyl 4-(4-(2-butyl-1-oxo-1,2-dihydro-2,7-naphthyridin-4-yl)phenoxy)piperidine-1-carboxylate (E42-4)

To a mixture of tert-butyl 4-(4-(4,4,5,5-tetramethyl-1,3,2-dioxaborolan-2-yl)phenoxy)piperidine-1-carboxylate (E42-3, 287 mg, 0.711 mmol), 4-bromo-2-butyl-2,7-naphthyridin-1(2H)-one (Intermediate 32, 200 mg, 0.711 mmol) and solid K₂CO₃ (196 mg, 1.422 mmol) in a mixture of water (5 ml) and 1,4-dioxane (15 ml) under an argon atmosphere was added Pd(PPh₃)₄ (82 mg, 0.0711 mmol) and the RM was stirred at 100° C. for 16 h. The mixture was concentrated, the residue was diluted with EtOAc (60 ml), the organic phase was washed with water (20 ml) and brine (20 ml), dried over Na₂SO₄ and the residue was purified by chromatography on silica gel eluting with EtOAc (from 0% to 30%) in DCM, yielding the title compound tert-butyl 4-(4-(2-butyl-1-oxo-1,2-dihydro-2,7-naphthyridin-4-yl)phenoxy)piperidine-1-carboxylate, E42-4, as a solid (300 mg).

Method LCMSA010: Rt=2.21 min; [M+H]⁺=478.

### Step 3: 2-butyl-4-(4-(piperidin-4-yloxy)phenyl)-2,7-naphthyridin-1(2H)-one (E42-5)

To a solution of tert-butyl 4-(4-(2-butyl-1-oxo-1,2-dihydro-2,7-naphthyridin-4-yl)phenoxy)piperidine-1-carboxylate (E42-4, 200 mg, 0.419 mmol) in MeOH (6 ml) was added a solution of HCl (4 M) in 1,4-dioxane (3.14 ml, 12.56 mmol) and the RM was stirred at RT for 4 h. The pH of the mixture was adjusted to pH=9 by the addition of an aq. solution of NaHCO₃, the mixture was concentrated and the residue was purified by chromatography on silica gel eluting with MeOH (from 0% to 33%) in DCM, yielding the title compound 2-butyl-4-(4-(piperidin-4-yloxy)phenyl)-2,7-naphthyridin-1(2H)-one, E42-5, as a solid (80 mg).

Method LCMSA010: Rt=1.83 min; [M+H]⁺=378.

### Step 4: tert-butyl 4-((4-(4-(2-butyl-1-oxo-1,2-dihydro-2,7-naphthyridin-4-yl)phenoxy)piperidin-1-yl)methyl)piperidine-1-carboxylate (E42-7)

To a solution of 2-butyl-4-(4-(piperidin-4-yloxy)phenyl)-2,7-naphthyridin-1(2H)-one (E42-5, 70 mg, 0.185 mmol) and tert-butyl 4-formylpiperidine-1-carboxylate (E42-6, 47 mg, 0.222 mmol) in DMSO (3 ml) was added a solution of ZnCl₂ (1 M) in THE (0.37 ml, 0.371 mmol) and the RM was stirred at RT for 30 min. Solid NaBH₃CN (23 mg, 0.371 mmol) and MeOH (1 ml) were added and the RM was stirred at RT for 16 h. The mixture was diluted with EtOAc (20 ml), the organic phase was washed with brine (3×15 ml), dried over Na₂SO₄ and the residue was purified by chromatography on silica gel eluting with EtOAc (from 0% to 30%) in DCM, yielding the title compound tert-butyl 4-((4-(4-(2-butyl-1-oxo-1,2-dihydro-2,7-naphthyridin-4-yl)phenoxy)piperidin-1-yl)methyl)piperidine-1-carboxylate, E42-7, as a solid (75 mg).

Method LCMSA043: Rt=2.62 min; [M+H]⁺=575.

### Step 5: 2-butyl-4-(4-((1-(piperidin-4-ylmethyl)piperidin-4-yl)oxy)phenyl)-2,7-naphthyridin-1(2H)-one (E42-8)

To a solution of tert-butyl 4-((4-(4-(2-butyl-1-oxo-1,2-dihydro-2,7-naphthyridin-4-yl)phenoxy)piperidin-1-yl)methyl)piperidine-1-carboxylate (E42-7, 75 mg, 0.13 mmol) in MeOH (3 ml) was added a solution of HCl (4 M) in 1,4-dioxane (0.65 ml, 2.6 mmol) and the RM was stirred at RT for 4 h. The pH of the mixture was adjusted to pH=9 by the addition of an aq-solution of NaHCO₃ and concentrated, yielding the title compound 2-butyl-4-(4-((1-(piperidin-4-ylmethyl)piperidin-4-yl)oxy)phenyl)-2,7-naphthyridin-1(2H)-one, E42-8, (60 mg), which was used for the next step without purification.

Method LCMSA022: Rt=0.86 min; [M+H]⁺=475.

### Step 6: 1-(5-(4-((4-(4-(2-butyl-1-oxo-1,2-dihydro-2,7-naphthyridin-4-yl)phenoxy)piperidin-1-yl)methyl)piperidine-1-carbonyl)-2-methoxyphenyl)dihydropyrimidine-2,4(1H,3H)-dione (Compound E42)

To a solution of 2-butyl-4-(4-((1-(piperidin-4-ylmethyl)piperidin-4-yl)oxy)phenyl)-2,7-naphthyridin-1(2H)-one (E42-8, 60 mg, 0.1264 mmol) and DIEA (49 mg, 0.38 mmol) in DMF (3 ml) was added perfluorophenyl 3-(2,4-dioxotetrahydropyrimidin-1(2H)-yl)-4-methoxybenzoate (Intermediate 26, 65 mg, 0.1517 mmol) and the RM was stirred at RT overnight. The mixture was diluted with EtOAc (60 ml), the organic phase was washed with brine (3×20 ml), dried over Na₂SO₄ and concentrated. The residue was purified by preparative HPLC, yielding the title compound 1-(5-(4-((4-(4-(2-butyl-1-oxo-1,2-dihydro-2,7-naphthyridin-4-yl)phenoxy)piperidin-1-yl)methyl)piperidine-1-carbonyl)-2-methoxyphenyl)dihydropyrimidine-2,4(1H,3H)-dione, Compound E42, as a solid (29 mg).

Method LCMSA043: Rt=1.95 min; [M+H]⁺=721.

¹H NMR (600 MHz, DMSO-d₆) δ[ppm] 10.34 (s, 1H), 9.43 (d, J=0.9 Hz, 1H), 8.70 (d, J=5.7 Hz, 1H), 7.74 (s, 1H), 7.41 (dd, J=5.6, 0.9 Hz, 1H), 7.39-7.34 (m, 3H), 7.32 (d, J=2.1 Hz, 1H), 7.16 (d, J=8.6 Hz, 1H), 7.11-7.05 (m, 2H), 4.53-4.22 (m, 2H), 4.03 (t, J=7.3 Hz, 2H), 3.85 (s, 3H), 3.80-3.66 (m, 1H), 3.60 (t, J=6.6 Hz, 2H), 3.19-2.63 (m, 6H), 2.28-2.14 (m, 4H), 2.02-1.93 (m, 2H), 1.86-1.59 (m, 7H), 1.38-1.29 (m, 2H), 1.13-1.02 (m, 2H), 0.91 (t, J=7.4 Hz, 3H).

### Compound E43: 1-(5-(4-((4-(4-(2-butyl-1-oxo-1,2-dihydro-2,7-naphthyridin-4-yl)-2-chlorophenoxy)piperidin-1-yl)methyl)piperidine-1-carbonyl)-2-methoxyphenyl)dihydropyrimidine-2,4(1H,3H)-dione

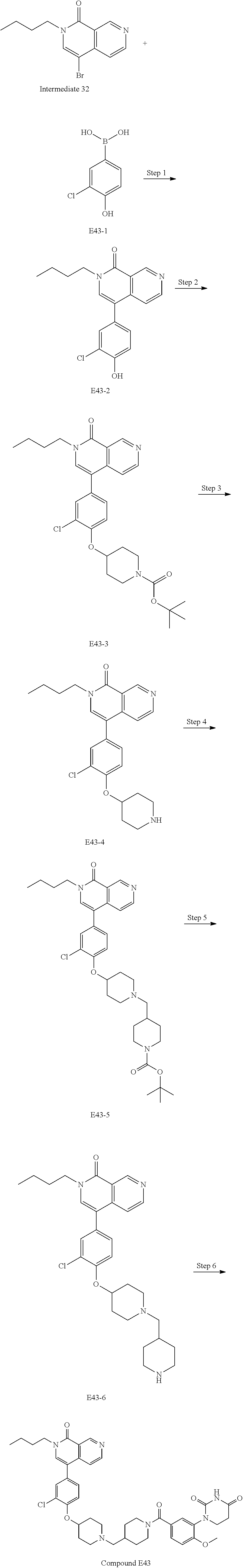

### Step 1: 2-butyl-4-(3-chloro-4-hydroxyphenyl)-2,7-naphthyridin-1(2H)-one (E43-2)

To a mixture of 4-bromo-2-butyl-2,7-naphthyridin-1(2H)-one (Intermediate 32, 200 mg, 0.71 mmol), (3-chloro-4-hydroxyphenyl)boronic acid (E43-1, 147 mg, 0.85 mmol), solid K₂CO₃ (196 mg, 1.42 mmol) in a mixture of water (5 ml) and 1,4-dioxane (10 ml) under a nitrogen atmosphere was added Pd(PPh₃)₄ (82 mg, 0.1 mmol) and the RM was stirred at 100° C. for 16 h. The mixture was concentrated and the residue was dissolved in EtOAc (60 ml). The organic phase was washed with water (20 ml) and brine (20 ml), dried over Na₂SO₄ and concentrated. The residue was purified by chromatography on silica gel eluting with EtOAc (from 0% to 30%) in DCM, yielding the title compound 2-butyl-4-(3-chloro-4-hydroxyphenyl)-2,7-naphthyridin-1(2H)-one, E43-2, as a solid (658 mg).

Method LCMSA027: Rt=1.68 min; [M+H]⁺=329.

### Step 2: tert-butyl 4-(4-(2-butyl-1-oxo-1,2-dihydro-2,7-naphthyridin-4-yl)-2-chlorophenoxy)piperidine-1-carboxylate (E43-3)

To a solution of 2-butyl-4-(3-chloro-4-hydroxyphenyl)-2,7-naphthyridin-1(2H)-one (E43-2, 260 mg, 0.79 mmol), tert-butyl 4-hydroxypiperidine-1-carboxylate (239 mg, 1.186 mmol) and PPh₃ in THE (15 ml) at 0° C. was added DEAD (275 mg, 1.58 mmol) and the RM was stirred at RT for 12 h. The mixture was diluted with EtOAc (40 ml), the organic phase was washed with water (20 ml) and brine (20 ml), dried over Na₂SO₄ and concentrated. The residue was purified by chromatography on silica gel eluting with EtOAc (from 0% to 20%) in DCM, yielding the title compound tert-butyl 4-(4-(2-butyl-1-oxo-1,2-dihydro-2,7-naphthyridin-4-yl)-2-chlorophenoxy)piperidine-1-carboxylate, E43-3, as a solid (300 mg).

Method LCMSA010: Rt=2.27 min; [M+H]⁺=512.

### Step 3: 2-butyl-4-(3-chloro-4-(piperidin-4-yloxy)phenyl)-2,7-naphthyridin-1(2H)-one (E43-4)

To a solution of tert-butyl 4-(4-(2-butyl-1-oxo-1,2-dihydro-2,7-naphthyridin-4-yl)-2-chlorophenoxy)piperidine-1-carboxylate (E43-3, 300 mg, 0.586 mmol) in MeOH (5 ml) was added a solution of HCl (4 M) in 1,4-dioxane (2.93 ml, 11.72 mmol) and the RM was stirred at RT for 3 h. The pH of the mixture was adjusted to pH=9 by the addition of a sat. aq. solution of NaHCO₃ and the mixture was concentrated. The residue was purified by chromatography on silica gel eluting with MeOH (from 0% to 33%) in DCM, yielding the title compound 2-butyl-4-(3-chloro-4-(piperidin-4-yloxy)phenyl)-2,7-naphthyridin-1(2H)-one, E43-4, as a solid (70 mg).

Method LCMSA010: Rt=1.99 min; [M+H]⁺=412.

### Step 4: tert-butyl 4-((4-(4-(2-butyl-1-oxo-1,2-dihydro-2,7-naphthyridin-4-yl)-2-chlorophenoxy)piperidin-1-yl)methyl)piperidine-1-carboxylate (E43-5)

To a solution of 2-butyl-4-(3-chloro-4-(piperidin-4-yloxy)phenyl)-2,7-naphthyridin-1(2H)-one (E43-4, 60 mg, 0.146 mmol) and tert-butyl 4-formylpiperidine-1-carboxylate (47 mg, 0.175 mmol) in DMSO (3 ml) was added a solution of ZnCl₂ (1 M) in THE (0.29 ml, 0.29 mmol) and the RM was stirred at RT for 30 min. Solid NaBH₃CN (18 mg, 0.291 mmol) and MeOH (1 ml) were added and the RM was stirred at RT for 16 h. The mixture was diluted with EtOAc (20 ml), the organic phase was washed with brine (3×15 ml), dried over Na₂SO₄ and concentrated. The residue was purified by chromatography on silica gel eluting with EtOAc (from 0% to 30%) in DCM, yielding the title compound tert-butyl 4-((4-(4-(2-butyl-1-oxo-1,2-dihydro-2,7-naphthyridin-4-yl)-2-chlorophenoxy)piperidin-1-yl)methyl)piperidine-1-carboxylate, E43-5, as a solid (70 mg).

Method LCMSA043: Rt=2.73 min; [M+H]⁺=609.

### Step 5: 2-butyl-4-(3-chloro-4-((1-(piperidin-4-ylmethyl)piperidin-4-yl)oxy)phenyl)-2,7-naphthyridin-1(2H)-one (E43-6)

To a solution of tert-butyl 4-((4-(4-(2-butyl-1-oxo-1,2-dihydro-2,7-naphthyridin-4-yl)-2-chlorophenoxy)piperidin-1-yl)methyl)piperidine-1-carboxylate (E43-5, 65 mg, 0.107 mmol) in MeOH (3 ml) was added a solution of HCl (4 M) in 1,4-dioxane (0.53 ml, 2.14 mmol) and the RM was stirred at RT for 4 h. The pH of the mixture was adjusted to pH=9 by the addition of a sat. aq. solution of NaHCO₃ and the mixture was concentrated, yielding the title compound 2-butyl-4-(3-chloro-4-((1-(piperidin-4-ylmethyl)piperidin-4-yl)oxy)phenyl)-2,7-naphthyridin-1(2H)-one, E43-6, as a solid (60 mg), which was used for the next step without further purification.

Method LCMSA022: Rt=0.88 min; [M+H]⁺=509.

### Step 6: 1-(5-(4-((4-(4-(2-butyl-1-oxo-1,2-dihydro-2,7-naphthyridin-4-yl)-2-chlorophenoxy)piperidin-1-yl)methyl)piperidine-1-carbonyl)-2-methoxyphenyl)dihydropyrimidine-2,4(1H,3H)-dione (Compound E43)

To a mixture of 2-butyl-4-(3-chloro-4-((1-(piperidin-4-ylmethyl)piperidin-4-yl)oxy)phenyl)-2,7-naphthyridin-1(2H)-one (E43-6, 60 mg) and DIEA (46 mg, 0.354 mmol) in DMF (3 ml) was added perfluorophenyl 3-(2,4-dioxotetrahydropyrimidin-1(2H)-yl)-4-methoxybenzoate (Intermediate 26, 61 mg, 0.141 mmol) and the RM was stirred at RT for 12 h. The mixture was diluted with EtOAc (60 ml), the organic phase was washed brine (3×20 ml), dried over Na₂SO₄ and concentrated. The residue was purified by preparative HPLC on an XBridge C18 column (250×21.2 mm, 10 μm) eluting with ACN (from 5% to 80%) in an aq. solution of NH₄HCO₃ (0.01 M), yielding the title compound 1-(5-(4-((4-(4-(2-butyl-1-oxo-1,2-dihydro-2,7-naphthyridin-4-yl)-2-chlorophenoxy)piperidin-1-yl)methyl)piperidine-1-carbonyl)-2-methoxyphenyl)dihydropyrimidine-2,4(1H,3H)-dione, Compound E43, as a solid (29 mg).

Method LCMSA043: Rt=2.04 min; [M+H]⁺=755.

¹H NMR (600 MHz, DMSO-d₆) δ[ppm] 10.34 (s, 1H), 9.43 (d, J=0.9 Hz, 1H), 8.72 (d, J=5.6 Hz, 1H), 7.81 (s, 1H), 7.54 (d, J=2.1 Hz, 1H), 7.41 (dd, J=5.6, 0.9 Hz, 1H), 7.37 (dt, J==8.5, 1.9 Hz, 2H), 7.34 (d, J=8.6 Hz, 1H), 7.32 (d, J=2.2 Hz, 1H), 7.16 (d, J=8.6 Hz, 1H), 4.58 (s, 1H), 4.41 (s, 1H), 4.02 (t, J=7.3 Hz, 2H), 3.84 (s, 3H), 3.72 (s, 1H), 3.60 (t, J=6.6 Hz, 2H), 3.23-2.56 (m, 6H), 2.28 (s, 2H), 2.18 (d, J=7.1 Hz, 2H), 1.96 (s, 2H), 1.86-1.61 (m, 7H), 1.33 (h, J=7.4 Hz, 2H), 1.07 (qd, J=12.6, 12.3, 4.1 Hz, 2H), 0.92 (t, J=7.4 Hz, 3H).

### Compound E45: 1-(5-(2-(4-((1-(4-(2-butyl-1-oxo-1,2-dihydro-2,7-naphthyridin-4-yl)-2,6-difluorobenzyl)piperidin-4-yl)oxy)piperidin-1-yl)-2-oxoethoxy)-2-methylphenyl)dihydropyrimidine-2,4(1H,3H)-dione

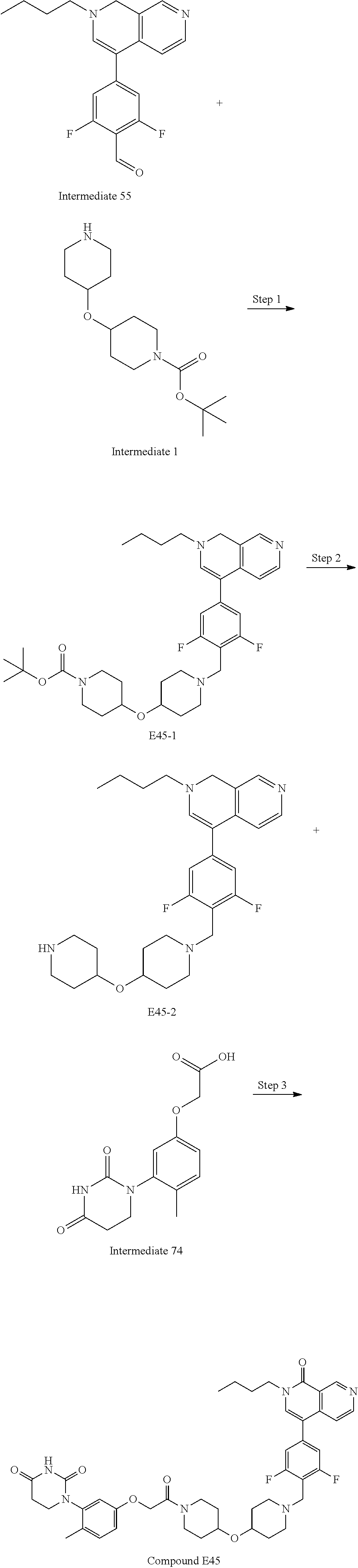 [SMILES: CCCCN1C=C(c2cc(F)c(C=O)c(F)c2)c2ccncc2C1 ; CC(C)(C)OC(=O)N1CCC(OC2CCNCC2)CC1 ; CCCCN1C=C(c2cc(F)c(CN3CCC(OC4CCN(C(=O)OC(C)(C)C)CC4)CC3)c(F)c2)c2ccncc2C1 ; CCCCN1C=C(c2cc(F)c(CN3CCC(OC4CCNCC4)CC3)c(F)c2)c2ccncc2C1 ; Cc1ccc(OCC(=O)O)cc1N1CCC(=O)NC1=O ; CCCCn1cc(-c2cc(F)c(CN3CCC(OC4CCN(C(=O)COc5ccc(C)c(N6CCC(=O)NC6=O)c5)CC4)CC3)c(F)c2)c2ccncc2c1=O]

### Step 1: tert-butyl 4-((1-(4-(2-butyl-1-oxo-1,2-dihydro-2,7-naphthyridin-4-yl)-2,6-difluorobenzyl)piperidin-4-yl)oxy)piperidine-1-carboxylate (E45-1)

To a solution of 4-(2-butyl-1-oxo-1,2-dihydro-2,7-naphthyridin-4-yl)-2,6-difluorobenzaldehyde (Intermediate 55, 400 mg, 1.17 mmol), TEA (0.500 ml, 3.59 mmol) and tert-butyl 4-(piperidin-4-yloxy)piperidine-1-carboxylate (Intermediate 1, 400 mg, 1.41 mmol) in MeOH (10 ml) under an argon atmosphere was added a solution of ZnCl₂ (0.5 M) in THE (3 ml, 1.50 mmol) and the RM was stirred at RT for 4 h. Solid NaBH₃CN (147 mg, 2.337 mmol) was added and the RM was stirred at RT for 16 h. The mixture was concentrated, yielding the title compound tert-butyl 4-((1-(4-(2-butyl-1-oxo-1,2-dihydro-2,7-naphthyridin-4-yl)-2,6-difluorobenzyl)piperidin-4-yl)oxy)piperidine-1-carboxylate, E45-1, as a solid, which was used for the next step without further purification.

Method LCMS_MLG2: Rt=0.91 min; [M+H]⁺=611.

### Step 2: 2-butyl-4-(3,5-difluoro-4-((4-(piperidin-4-yloxy)piperidin-1-yl)methyl)phenyl)-2,7-naphthyridin-1(2H)-one (E45-2)

To a solution of tert-butyl 4-((1-(4-(2-butyl-1-oxo-1,2-dihydro-2,7-naphthyridin-4-yl)-2,6-difluorobenzyl)piperidin-4-yl)oxy)piperidine-1-carboxylate (E45-1, 1.17 mmol) in DCM (10 ml) was added TFA (3 ml, 38.9 mmol) and the RM was stirred at RT for 1 h. The mixture was concentrated and the residue was purified by reverse phase chromatography on an Isco REDISEP® Gold HP C18 column (15.5 g) eluting with ACN (from 1% to 100%) in an aq. solution of TFA (0.1%), yielding the title compound 2-butyl-4-(3,5-difluoro-4-((4-(piperidin-4-yloxy)piperidin-1-yl)methyl)phenyl)-2,7-naphthyridin-1(2H)-one, E45-2, as a solid TFA salt (449 mg).

Method LCMS_MLG2: Rt=0.34 min; [M+H]⁺=511.

### Step 3: 1-(5-(2-(4-((1-(4-(2-butyl-1-oxo-1,2-dihydro-2,7-naphthyridin-4-yl)-2,6-difluorobenzyl)piperidin-4-yl)oxy)piperidin-1-yl)-2-oxoethoxy)-2-methylphenyl)dihydropyrimidine-2,4(1H,3H)-dione (Compound E45)

To a solution of 2-(3-(2,4-dioxotetrahydropyrimidin-1(2H)-yl)-4-methylphenoxy)acetic acid (Intermediate 74, 31 mg, 0.111 mmol) in DMF (1.5 ml) were added DIEA (0.050 ml, 0.286 mmol) and HATU (46 mg, 0.121 mmol) and the RM was stirred at RT for 30 min. Solid 2-butyl-4-(3,5-difluoro-4-((4-(piperidin-4-yloxy)piperidin-1-yl)methyl)phenyl)-2,7-naphthyridin-1(2H)-one TFA salt (E45-2, 95 mg, 0.129 mmol) was added and the RM was stirred at RT for 4 h. the mixture was concentrated and the residue was purified by reverse phase chromatography on a REDISEP® Gold HP C18 column (15.5 g) eluting with ACN (from 2% to 100%) in an aq. solution of TFA (0.1%). Fractions containing the title compound were combined and concentrated. The residue was purified by reverse phase chromatography on a REDISEP® Gold HP C18 column (15.5 g) eluting with ACN (from 1% to 100%) in an aq. solution of NH₄HCO₃ (0.1%), yielding the title compound 1-(5-(2-(4-((1-(4-(2-butyl-1-oxo-1,2-dihydro-2,7-naphthyridin-4-yl)-2,6-difluorobenzyl)piperidin-4-yl)oxy)piperidin-1-yl)-2-oxoethoxy)-2-methylphenyl)dihydropyrimidine-2,4(1H,3H)-dione, Compound E45, as a solid (73 mg).

Method LCMS_MLG4: Rt=2.67 min; [M+H]⁺=772.

¹H NMR (600 MHz, DMSO-d₆) δ[ppm] 10.33 (s, 1H), 9.46 (s, 1H), 8.77 (d, J=5.7 Hz, 1H), 7.94 (s, 1H), 7.50 (m, 3H), 7.16 (d, J=8.5 Hz, 1H), 6.88 (d, J=2.7 Hz, 1H), 6.80 (dd, J=8.5, 2.7 Hz, 1H), 4.76 (s, 2H), 4.48 (m, 1H), 4.04 (t, J=7.4 Hz, 2H), 3.89-3.42 (m, 9H), 3.26-3.15 (m, 3H), 2.82-2.56 (m, 3H), 2.09 (s, 3H), 2.07-1.75 (m, 5H), 1.74-1.69 (m, 2H), 1.53 (m, 3H), 1.37-1.31 (m, 2H), 0.92 (t, J=7.4 Hz, 3H).

### Compound E46: 1-(4-(2-(4-((1-(2,5-dimethoxy-4-(2-methyl-1-oxo-1,2-dihydro-2,7-naphthyridin-4-yl)benzyl)piperidin-4-yl)oxy)piperidin-1-yl)-2-oxoethoxy)phenyl)dihydropyrimidine-2,4(1H,3H)-dione

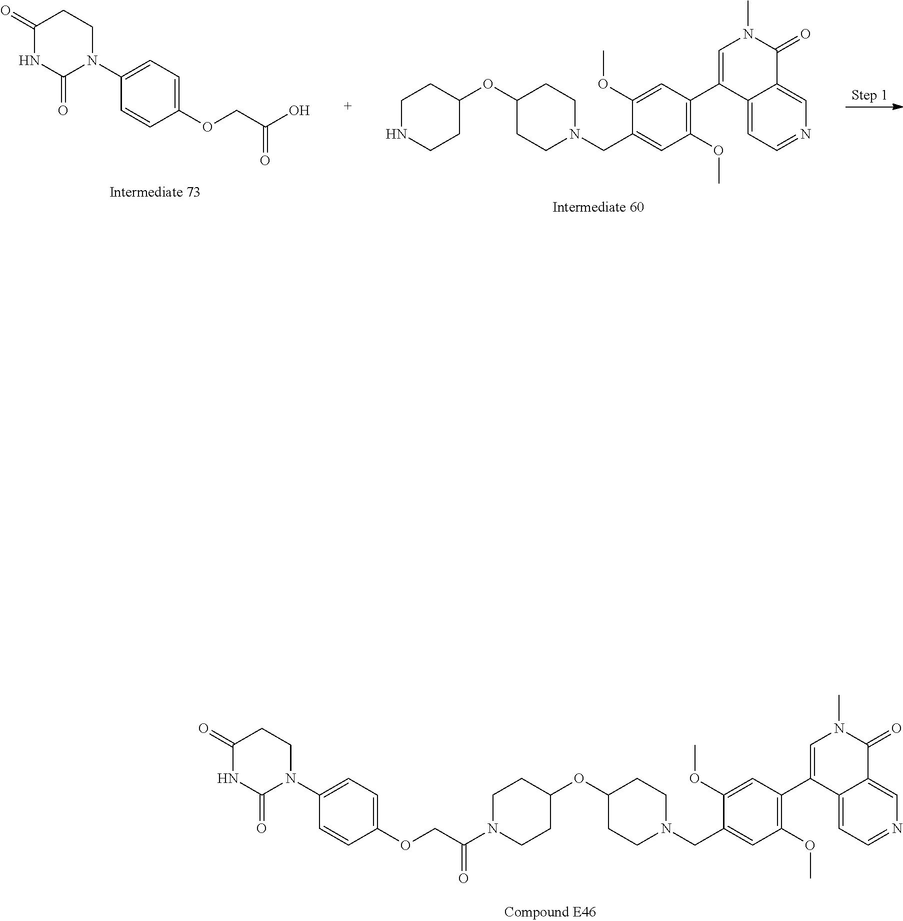 [SMILES: O=C(O)COc1ccc(N2CCC(=O)NC2=O)cc1 ; COc1cc(-c2cn(C)c(=O)c3cnccc23)c(OC)cc1CN1CCC(OC2CCNCC2)CC1 ; COc1cc(-c2cn(C)c(=O)c3cnccc23)c(OC)cc1CN1CCC(OC2CCN(C(=O)COc3ccc(N4CCC(=O)NC4=O)cc3)CC2)CC1]

### Step 1: 1-(4-(2-(4-((1-(2,5-dimethoxy-4-(2-methyl-1-oxo-1,2-dihydro-2,7-naphthyridin-4-yl)benzyl)piperidin-4-yl)oxy)piperidin-1-yl)-2-oxoethoxy)phenyl)dihydropyrimidine-2,4(1H,3H)-dione (Compound E46)

To a solution of 2-(4-(2,4-dioxotetrahydropyrimidin-1(2H)-yl)phenoxy)acetic acid (Intermediate 73, 90 mg, 0.34 mmol), 4-(2,5-dimethoxy-4-((4-(piperidin-4-yloxy)piperidin-1-yl)methyl)phenyl)-2-methyl-2,7-naphthyridin-1(2H)-one (Intermediate 60, 200 mg, 0.34 mmol) and DIEA (219 mg, 1.7 mmol) in DMF (2 ml) was added HATU (155 mg, 0.408 mmol) and the RM was stirred at RT for 1 h. The mixture was purified by preparative HPLC on an XBridge C18 column (250×21.2 mm, 10 μm) eluting with ACN (from 5% to 95%) in an aq. solution of NH₄HCO₃ (0.01 M), yielding the title compound 1-(4-(2-(4-((1-(2,5-dimethoxy-4-(2-methyl-1-oxo-1,2-dihydro-2,7-naphthyridin-4-yl)benzyl)piperidin-4-yl)oxy)piperidin-1-yl)-2-oxoethoxy)phenyl)dihydropyrimidine-2,4(1H,3H)-dione, Compound E46, as a solid (45 mg).

Method LCMSA039: Rt=1.63 min; [M+H]⁺=739.

¹H NMR (500 MHz, DMSO-d₆) δ[ppm] 10.31 (s, 1H), 9.41 (s, 1H), 8.65 (d, J=5.6 Hz, 1H), 7.74 (s, 1H), 7.22 (d, J=8.9 Hz, 2H), 7.15 (s, 1H), 7.03 (d, J=5.7 Hz, 1H), 6.92 (d, J=8.9 Hz, 3H), 4.81 (s, 2H), 3.88-3.79 (m, 1H), 3.77-3.60 (m, 10H), 3.57 (s, 3H), 3.55-3.43 (m, 3H), 3.22 (t, J=10.7 Hz, 1H), 3.10 (t, J=10.1 Hz, 1H), 2.86-2.71 (m, 2H), 2.69 (t, J=6.7 Hz, 2H), 2.29-2.08 (m, 2H), 1.92-1.71 (m, 4H), 1.56-1.28 (m, 4H).

### Compound E48: 1-(2-chloro-5-(4-((4-(4-(4,5-dimethyl-6-oxo-1-propyl-1,6-dihydropyridin-3-yl)-2,6-dimethoxyphenethyl)piperazin-1-yl)methyl)piperidine-1-carbonyl)phenyl)dihydropyrimidine-2,4(1H,3H)-dione

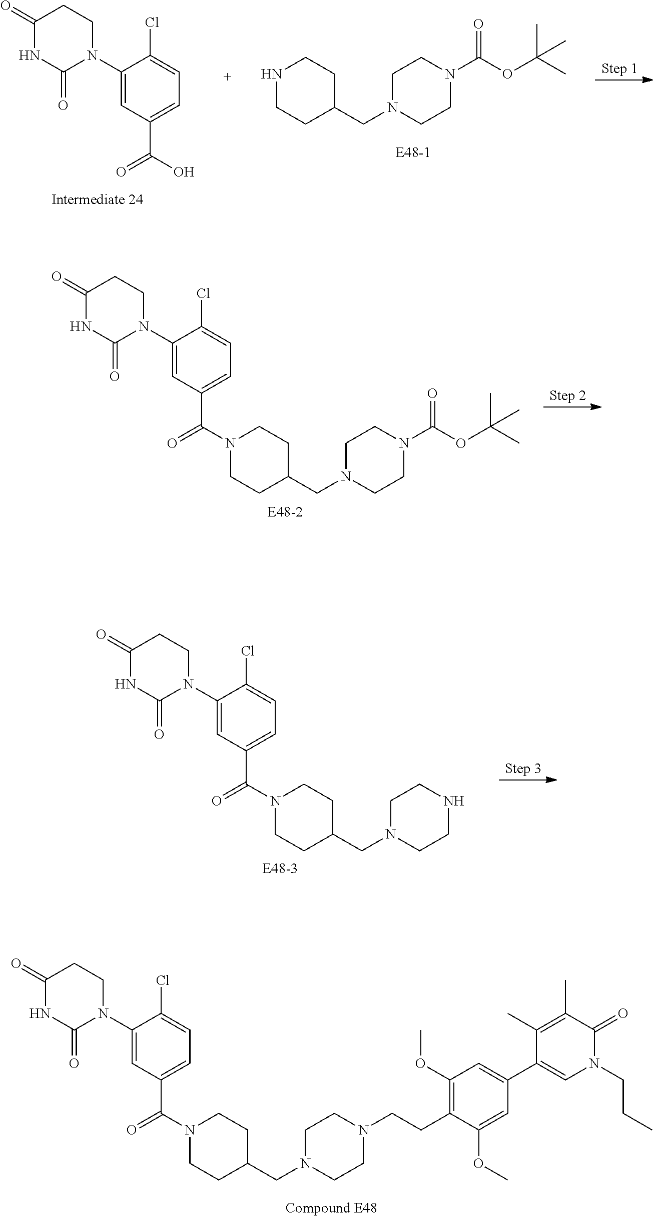 [SMILES: O=C1CCN(c2cc(C(=O)O)ccc2Cl)C(=O)N1 ; CC(C)(C)OC(=O)N1CCN(CC2CCNCC2)CC1 ; CC(C)(C)OC(=O)N1CCN(CC2CCN(C(=O)c3ccc(Cl)c(N4CCC(=O)NC4=O)c3)CC2)CC1 ; O=C1CCN(c2cc(C(=O)N3CCC(CN4CCNCC4)CC3)ccc2Cl)C(=O)N1 ; CCCn1cc(-c2cc(OC)c(CCN3CCN(CC4CCN(C(=O)c5ccc(Cl)c(N6CCC(=O)NC6=O)c5)CC4)CC3)c(OC)c2)c(C)c(C)c1=O]

### Step 1: tert-butyl 4-((1-(4-chloro-3-(2,4-dioxotetrahydropyrimidin-1(2H)-yl)benzoyl)piperidin-4-yl)methyl)piperazine-1-carboxylate (E48-2)

To a solution of 4-chloro-3-(2,4-dioxotetrahydropyrimidin-1(2H)-yl)benzoic acid (Intermediate 24, 400 mg, 1.49 mmol) in DMF (5 ml) were added DIEA (385.13 mg, 2.98 mmol) and HATU (680.61 mg, 1.79 mmol) and the RM was stirred at 10° C. for 1 h. Tert-butyl 4-(piperidin-4-ylmethyl)piperazine-1-carboxylate (E48-1, 506.37 mg, 1.79 mmol) was added and the RM was stirred at 10° C. for 11 h. The mixture was added into water (20 ml) and the aq. phase was extracted with EtOAc (4×20 ml). The combined organic phases were washed with brine (2×15 ml), dried over Na₂SO₄ and concentrated. The residue was triturated with a mixture of MeOH (15 ml) and DMSO (1 ml). The mixture was filtered and the solids were collected, yielding the title compound tert-butyl 4-((1-(4-chloro-3-(2,4-dioxotetrahydropyrimidin-1(2H)-yl)benzoyl)piperidin-4-yl)methyl)piperazine-1-carboxylate, E48-2, as a solid (300 mg). The filtrate was concentrated and purified by preparative HPLC on a Phenomenex Synergi C18 column (150×25 mm, 10 μm) eluting with ACN (from 10% to 40%) in an aq. solution of TFA (0.1%), yielding the title compound tert-butyl 4-((1-(4-chloro-3-(2,4-dioxotetrahydropyrimidin-1(2H)-yl)benzoyl)piperidin-4-yl)methyl)piperazine-1-carboxylate, E48-2, as a solid TFA salt (150 mg).

Method LCMS WX2: Rt=0.96 min; [M+H]⁺=534.

### Step 2: 1-(2-chloro-5-(4-(piperazin-1-ylmethyl)piperidine-1-carbonyl)phenyl)dihydropyrimidine-2,4(1H,3H)-dione (E48-3)

To a solution of tert-butyl 4-((1-(4-chloro-3-(2,4-dioxotetrahydropyrimidin-1(2H)-yl)benzoyl)piperidin-4-yl)methyl)piperazine-1-carboxylate TFA salt (E48-2, 150 mg, 0.256 mmol) in DCM (10 ml) was added a solution of HCl (1 M) in 1,4-dioxane (10 ml, 10 mmol) and the RM was stirred at 10° C. for 2 h. The mixture was concentrated, yielding the title compound 1-(2-chloro-5-(4-(piperazin-1-ylmethyl)piperidine-1-carbonyl)phenyl)dihydropyrimidine-2,4(1H,3H)-dione, E48-3, as a solid HCl salt (130 mg), which was used for the next step without further purification.

Method LCMS WX4: Rt=1.07 min; [M+H]⁺=434.

### Step 3: 1-(2-chloro-5-(4-((4-(4-(4,5-dimethyl-6-oxo-1-propyl-1,6-dihydropyridin-3-yl)-2,6-dimethoxyphenethyl)piperazin-1-yl)methyl)piperidine-1-carbonyl)phenyl)dihydropyrimidine-2,4(1H,3H)-dione (Compound E48)

To a solution of 2-(4-(4,5-dimethyl-6-oxo-1-propyl-1,6-dihydropyridin-3-yl)-2,6-dimethoxyphenyl)acetaldehyde (Intermediate 52, 100 mg, 0.291 mmol) and 1-(2-chloro-5-(4-(piperazin-1-ylmethyl)piperidine-1-carbonyl)phenyl)dihydropyrimidine-2,4(1H,3H)-dione HCl salt (E48-3, 126.3 mg, 0.291 mmol) in a mixture of THE (2 ml) and EtOH (2 ml) were added DIEA (150.4 mg, 1.164 mmol) and a solution of ZnCl₂ (2 M) in THE (0.29 ml, 0.58 mmol) and the RM was stirred at 10° C. for 1 h. Solid NaBH₃CN (36.60 mg, 0.582 mmol) was added and the RM was stirred at 10° C. for 15 h. The mixture was added into water (15 ml), the aq. phase was extracted with DCM (3×15 ml), the combined organic phases were washed with brine (2×10 ml), dried over Na₂SO₄ and concentrated. The residue was purified by preparative HPLC on a Phenomenex Luna C18 column (150×40 mm, 15 μm) eluting with ACN (from 5% to 45%) in an aq. solution of TFA (0.1%). Fractions containing the title compound were combined and concentrated. The pH of the residue was adjusted to pH=8-9 by the addition of an aq. solution of NaHCO₃ and the mixture was extracted with DCM (4×15 ml). The combined organic phases were washed with brine (2×10 ml), dried over Na₂SO₄ and concentrated, yielding the title compound 1-(2-chloro-5-(4-((4-(4-(4,5-dimethyl-6-oxo-1-propyl-1,6-dihydropyridin-3-yl)-2,6-dimethoxyphenethyl)piperazin-1-yl)methyl)piperidine-1-carbonyl)phenyl)dihydropyrimidine-2,4(1H,3H)-dione, Compound E48, as a solid (74.85 mg).

Method LCMS WX2: Rt=0.76 min; [M+H]⁺=761.

¹H NMR (400 MHz, DMSO-d₆) δ[ppm] 10.52 (br s, 1H), 7.64 (d, J=8.2 Hz, 1H), 7.55 (d, J=2.0 Hz, 1H), 7.45 (s, 1H), 7.39 (dd, J=2.0, 8.2 Hz, 1H), 6.51 (s, 2H), 4.49-4.38 (m, 1H), 3.86 (br t, J=7.4 Hz, 2H), 3.77 (m, 7H), 3.67-3.56 (m, 2H), 3.11-2.97 (m, 1H), 2.85-2.61 (m, 6H), 2.36-2.27 (m, 4H), 2.14 (br d, J=7.0 Hz, 2H), 2.05 (m, 6H), 1.88-1.73 (m, 2H), 1.72-1.58 (m, 4H), 1.24 (br s, 2H), 1.07 (br d, J=9.6 Hz, 4H), 0.87 (t, J=7.4 Hz, 3H).

### Compound E49: 1-(5-(4-((4-(4-(2-butyl-1-oxo-1,2-dihydro-2,7-naphthyridin-4-yl)-2,6-difluorophenoxy)piperidin-1-yl)methyl)piperidine-1-carbonyl)-2-methoxyphenyl)dihydropyrimidine-2,4(1H,3H)-dione

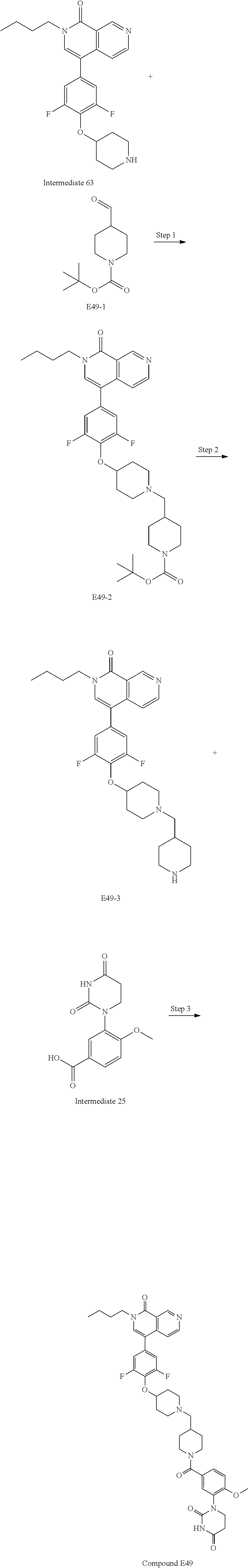 [SMILES: CCCCn1cc(-c2cc(F)c(OC3CCNCC3)c(F)c2)c2ccncc2c1=O ; CC(C)(C)OC(=O)N1CCC(C=O)CC1 ; CCCCn1cc(-c2cc(F)c(OC3CCN(CC4CCN(C(=O)OC(C)(C)C)CC4)CC3)c(F)c2)c2ccncc2c1=O ; CCCCn1cc(-c2cc(F)c(OC3CCN(CC4CCNCC4)CC3)c(F)c2)c2ccncc2c1=O ; COc1ccc(C(=O)O)cc1N1CCC(=O)NC1=O ; CCCCn1cc(-c2cc(F)c(OC3CCN(CC4CCN(C(=O)c5ccc(OC)c(N6CCC(=O)NC6=O)c5)CC4)CC3)c(F)c2)c2ccncc2c1=O]

### Step 1: tert-butyl 4-((4-(4-(2-butyl-1-oxo-1,2-dihydro-2,7-naphthyridin-4-yl)-2,6-difluorophenoxy)piperidin-1-yl)methyl)piperidine-1-carboxylate (E49-2)

To a mixture of 2-butyl-4-(3,5-difluoro-4-(piperidin-4-yloxy)phenyl)-2,7-naphthyridin-1(2H)-one TFA salt (Intermediate 63, 85 mg, 0.162 mmol), TEA (0.075 ml, 0.538 mmol) and tert-butyl 4-formylpiperidine-1-carboxylate (E49-1, 44 mg, 0.206 mmol) in MeOH (2 ml) under an argon atmosphere was added a solution of ZnCl₂ (0.7M) in THE (0.300 ml, 0.210 mmol) and the RM was stirred at RT for 7 h. Solid NaBH₃CN (18 mg, 0.286 mmol) was added and the RM was stirred at RT for 16 h. The mixture was concentrated, yielding the title compound tert-butyl 4-((4-(4-(2-butyl-1-oxo-1,2-dihydro-2,7-naphthyridin-4-yl)-2,6-difluorophenoxy)piperidin-1-yl)methyl)piperidine-1-carboxylate, E49-2, as a solid (109 mg), which was used for the next step without further purification.

Method LCMS_MLG9: Rt=0.80 min; [M+H]⁺=611.

### Step 2: 2-butyl-4-(3,5-difluoro-4-((1-(piperidin-4-ylmethyl)piperidin-4-yl)oxy)phenyl)-2,7-naphthyridin-1(2H)-one (E49-3)

To a solution of tert-butyl 4-((4-(4-(2-butyl-1-oxo-1,2-dihydro-2,7-naphthyridin-4-yl)-2,6-difluorophenoxy)piperidin-1-yl)methyl)piperidine-1-carboxylate (E49-2, 99 mg, 0.162 mmol) in DCM (1.5 ml) was added TFA (0.350 ml, 4.54 mmol) and the RM was stirred at RT for 1 h. The mixture was concentrated and the residue was purified by reverse phase chromatography on an Isco REDISEP® Gold HP C18 column (15.5 g) eluting with ACN (from 1% to 100%) in an aq. solution of TFA (0.1%), yielding the title compound 2-butyl-4-(3,5-difluoro-4-((1-(piperidin-4-ylmethyl)piperidin-4-yl)oxy)phenyl)-2,7-naphthyridin-1(2H)-one, E49-3, as a solid TFA salt (110 mg).

Method LCMS_MLG9: Rt=0.56 min; [M+H]⁺=511.

### Step 3: 1-(5-(4-((4-(4-(2-butyl-1-oxo-1,2-dihydro-2,7-naphthyridin-4-yl)-2,6-difluorophenoxy)piperidin-1-yl)methyl)piperidine-1-carbonyl)-2-methoxyphenyl)dihydropyrimidine-2,4(1H,3H)-dione (Compound E49)

To a solution of 3-(2,4-dioxotetrahydropyrimidin-1(2H)-yl)-4-methoxybenzoic acid (Intermediate 25, 45 mg, 0.170 mmol) in DMF (0.5 ml) were added DIEA (0.075 ml, 0.429 mmol) and HATU (70 mg, 0.184 mmol) and the RM was stirred at RT for 30 min. A solution of 2-butyl-4-(3,5-difluoro-4-((1-(piperidin-4-ylmethyl)piperidin-4-yl)oxy)phenyl)-2,7-naphthyridin-1(2H)-one TFA salt (E49-3, 110 mg, 0.141 mmol) and DIEA (0.075 ml, 0.429 mmol) in DMF (1 ml) was added and the RM was stirred at RT for 2 h. The mixture was purified by reverse phase chromatography on an Isco REDISEP® Gold HP C18 column (15.5 g) eluting with ACN (from 1% to 100%) in an aq. solution of TFA (0.1%). Fractions containing the title compound were combined, concentrated and the residue was purified by reverse phase chromatography on an Isco REDISEP® Gold HP C18 column (15.5 g) eluting with ACN (from 1% to 100%) in an aq. solution of NH₄HCO₃ (0.1%), yielding the title compound 1-(5-(4-((4-(4-(2-butyl-1-oxo-1,2-dihydro-2,7-naphthyridin-4-yl)-2,6-difluorophenoxy)piperidin-1-yl)methyl)piperidine-1-carbonyl)-2-methoxyphenyl)dihydropyrimidine-2,4(1H,3H)-dione, Compound E49, as a solid (43 mg).

Method LCMS_MLG2: Rt=0.66 min; [M+H]⁺=757.

¹H NMR (600 MHz, DMSO-d₆) δ[ppm] 10.33 (s, 1H), 9.43 (s, 1H), 8.74 (d, J=5.7 Hz, 1H), 7.88 (s, 1H), 7.50 (d, J=5.6 Hz, 1H), 7.36 (dd, J=8.5, 2.2 Hz, 1H), 7.32 (m, 3H), 7.15 (d, J=8.6 Hz, 1H), 4.42 (m, 1H), 4.22 (m, 1H), 4.02 (t, J=7.4 Hz, 2H), 3.84 (m, 4H), 3.60 (t, J=6.7 Hz, 2H), 3.24-2.63 (m, 5H), 2.17 (m, 4H), 1.93 (m, 2H), 1.85-1.64 (m, 7H), 1.33 (m, 2H), 1.06 (m, 2H), 0.92 (t, J=7.4 Hz, 3H).

### Compound E52: 1-(2-Chloro-5-(4-((1-(4-(4,5-dimethyl-6-oxo-1-propyl-1,6-dihydropyridin-3-yl)-2-fluoro-6-methoxyphenethyl)piperidin-4-yl)oxy)piperidine-1-carbonyl)phenyl)dihydropyrimidine-2,4(1H,3H)-dione

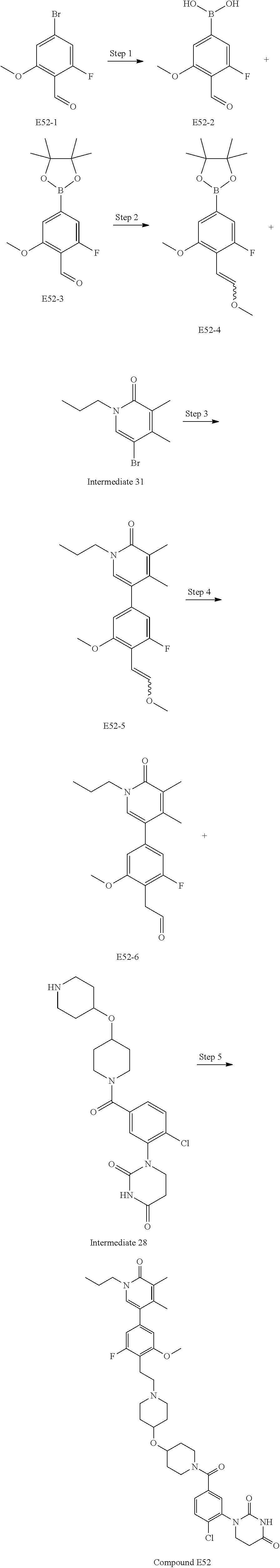

### Step 1: Mixture of (3-fluoro-4-formyl-5-methoxyphenyl)boronic Acid (E52-2) and

### 2-fluoro-6-methoxy-4-(4,4,5,5-tetramethyl-1,3,2-dioxaborolan-2-yl)benzaldehyde (E52-3)

To a mixture of 4-bromo-2-fluoro-6-methoxybenzaldehyde (E52-1, 707 mg, 2.97 mmol), BISPIN (906 mg, 3.57 mmol), dppf (50 mg, 0.090 mmol) and KOAc (875 g, 8.92 mmol) in 1,4-dioxane (15 ml) under an argon atmosphere was added PdCl₂(dppf) (66 mg, 0.090 mmol) and the RM was heated at 90° C. for 2 days. The RM was cooled to RT, solid PdCl₂(dppf) (66 mg, 0.090 mmol) was added and the RM was heated at 100° C. for 4 h. The mixture was filtered over CELITE® and the solids were washed with EtOAc. The filtrate was washed with an aq. solution of HCl (0.1 M) and brine, the organic phase was dried over MgSO₄ and concentrated. The residue was purified on an Isco REDISEP® silica column (40 g) eluting with EtOAc (from 0% to 80%) in CHX, yielding a mixture of the title compounds (3-fluoro-4-formyl-5-methoxyphenyl)boronic acid, E52-2, and 2-fluoro-6-methoxy-4-(4,4,5,5-tetramethyl-1,3,2-dioxaborolan-2-yl)benzaldehyde, E52-3, as a solid (612 mg).

Method LCMS_MLG1: Rt=0.64 min; [M+H]⁺=199 (E52-2) and Rt=1.30 min; [M+H]⁺=281 (E52-3).

### Step 2: (E,Z)-2-(3-fluoro-5-methoxy-4-(2-methoxyvinyl)phenyl)-4,4,5,5-tetramethyl-1,3,2-dioxaborolane (E52-4)

To a solution of methoxymethyltriphenylphosphonium chloride (1.13 g, 3.30 mmol) in THE (10 ml) was added a solution of tBuOK (1.0 M) in THE (4 ml, 4.00 mmol) and the RM was stirred at 0° C. for 30 min. A mixture of (3-fluoro-4-formyl-5-methoxyphenyl)boronic acid and 2-fluoro-6-methoxy-4-(4,4,5,5-tetramethyl-1,3,2-dioxaborolan-2-yl)benzaldehyde (E52-2 and E52-3, 375 mg) was added, the RM was stirred at RT for 2 h and heated at 65° C. for 2 days. The mixture was added into water and the aq. phase was extracted with EtOAc. The organic phase was washed with water and brine, dried over MgSO₄, concentrated and the residue was purified on an Isco REDISEP® silica column (40 g) eluting with EtOAc (from 0% to 50%) in CHX, yielding the title compounds (E,Z)-2-(3-fluoro-5-methoxy-4-(2-methoxyvinyl)phenyl)-4,4,5,5-tetramethyl-1,3,2-dioxaborolane, E52-4, as a solid (33 mg).

Method LCMS_MLG8: Rt=1.27 and 1.42 min; [M+H]⁺=309.

### Step 3: (E,Z)-5-(3-fluoro-5-methoxy-4-(2-methoxyvinyl)phenyl)-3,4-dimethyl-1-propylpyridin-2(1H)-one (E52-5)

To a mixture of 5-bromo-3,4-dimethyl-1-propylpyridin-2(1H)-one, (Intermediate 31, 27 mg, 0.111 mmol), solid K₂CO₃ (45 mg, 0.326 mmol) and (E,Z)-2-(3-fluoro-5-methoxy-4-(2-methoxyvinyl)phenyl)-4,4,5,5-tetramethyl-1,3,2-dioxaborolane, (E52-4, 33 mg, 0.107 mmol) in a mixture of ACN (2.0 ml) and water (0.5 ml) under an argon atmosphere was added Pd(dppf)Cl₂ (8 mg, 0.011 mmol) and the RM was heated at 100° C. for 1 h. The mixture was filtered over CELITE®, the filtrate was concentrated and the residue was purified by reverse phase chromatography on an Isco REDISEP® Gold HP C18 column (15.5 g) eluting with ACN (from 1% to 100%) in an aq. solution of TFA (0.1%), yielding the title compounds (E,Z)-5-(3-fluoro-5-methoxy-4-(2-methoxyvinyl)phenyl)-3,4-dimethyl-1-propylpyridin-2(1H)-one, E52-5, as a solid (26 mg).

Method LCMS_MLG8: Rt=1.08 and 1.20 min; [M+H]⁺=346.

### Step 4: 2-(4-(4,5-dimethyl-6-oxo-1-propyl-1,6-dihydropyridin-3-yl)-2-fluoro-6-methoxyphenyl)acetaldehyde (E52-6)

To a solution of (E,Z)-5-(3-fluoro-5-methoxy-4-(2-methoxyvinyl)phenyl)-3,4-dimethyl-1-propylpyridin-2(1H)-one (E52-5, 26 mg, 0.73 mmol) in acetone (1 ml) was added an aq. solution of HCl (2.0 M, 0.300 ml, 0.600 mmol) and the RM was heated at 65° C. for 1 h. The mixture was concentrated, yielding the title compound 2-(4-(4,5-dimethyl-6-oxo-1-propyl-1,6-dihydropyridin-3-yl)-2-fluoro-6-methoxyphenyl)acetaldehyde, E52-6, as a solid (26.6 mg), which was used for the next step without further purification.

Method LCMS_MLG8: Rt=0.96 min; [M+H]⁺=332.

### Step 5: 1-(2-chloro-5-(4-((1-(4-(4,5-dimethyl-6-oxo-1-propyl-1,6-dihydropyridin-3-yl)-2-fluoro-6-methoxyphenethyl)piperidin-4-yl)oxy)piperidine-1-carbonyl)phenyl)dihydropyrimidine-2,4(1H,3H)-dione (Compound E52)

To a solution of 2-(4-(4,5-dimethyl-6-oxo-1-propyl-1,6-dihydropyridin-3-yl)-2-fluoro-6-methoxyphenyl)acetaldehyde (E52-6, 24.2 mg, 0.073 mmol), TEA (0.035 ml, 0.251 mmol) and 1-(2-chloro-5-(4-(piperidin-4-yloxy)piperidine-1-carbonyl)phenyl)dihydropyrimidine-2,4(1H,3H)-dione HCl salt (Intermediate 28, 44 mg, 0.088 mmol) in MeOH (1 ml) under an argon atmosphere was added a solution of ZnCl₂ (0.5 M) in THE (0.175 ml, 0.088 mmol) and the RM was stirred at RT for 2 h. Solid NaBH₃CN (9 mg, 0.143 mmol) was added and the RM was stirred at RT for 4 days. The mixture was concentrated and the residue was purified by reverse phase chromatography on an Isco REDISEP® Gold HP C18 column (15.5 g) eluting with ACN (from 1% to 100%) in an aq. solution of NH₄HCO₃ (0.1%), yielding the title compound 1-(2-chloro-5-(4-((1-(4-(4,5-dimethyl-6-oxo-1-propyl-1,6-dihydropyridin-3-yl)-2-fluoro-6-methoxyphenethyl)piperidin-4-yl)oxy)piperidine-1-carbonyl)phenyl)dihydropyrimidine-2,4(1H,3H)-dione, Compound E52, as a solid (22 mg).

Method LCMS_MLG4: Rt=0.63 min; [M+H]⁺=751.

### Compound E54: 1-(5-(4-((1-(4-(2-butyl-1-oxo-1,2-dihydro-2,7-naphthyridin-4-yl)-2-fluorobenzyl)piperidin-4-yl)oxy)piperidine-1-carbonyl)-2-methoxyphenyl)dihydropyrimidine-2,4(1H,3H)-dione

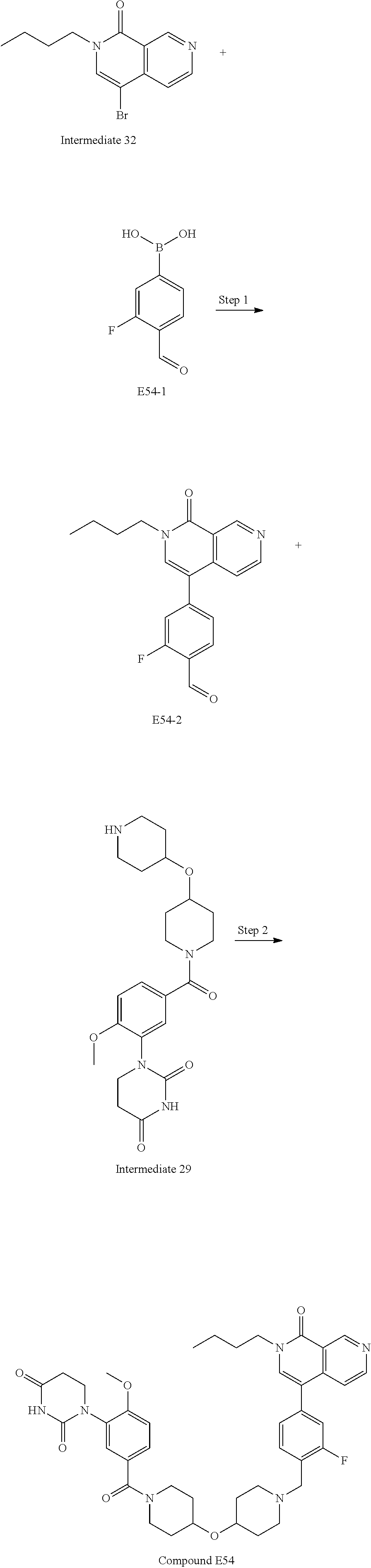 [SMILES: CCCCn1cc(Br)c2ccncc2c1=O ; O=Cc1ccc(B(O)O)cc1F ; CCCCn1cc(-c2ccc(C=O)c(F)c2)c2ccncc2c1=O ; COc1ccc(C(=O)N2CCC(OC3CCNCC3)CC2)cc1N1CCC(=O)NC1=O ; CCCCn1cc(-c2ccc(CN3CCC(OC4CCN(C(=O)c5ccc(OC)c(N6CCC(=O)NC6=O)c5)CC4)CC3)c(F)c2)c2ccncc2c1=O]

### Step 1: 4-(2-butyl-1-oxo-1,2-dihydro-2,7-naphthyridin-4-yl)-2-fluorobenzaldehyde (E54-2)

To a solution of 4-bromo-2-butyl-2,7-naphthyridin-1(2H)-one (Intermediate 32, 300 mg, 1.07 mmol), 3-fluoro-4-formylphenylboronic acid (E54-1, 200 mg, 1.28 mmol) and Na₂CO₃ (339 mg, 3.20 mmol) in a mixture of 1,4-dioxane (8 ml) and water (2 ml) under an argon atmosphere was added PdCl₂(dppf)-CH₂Cl₂ (79 mg, 0.107 mmol) and the RM was heated at 100° C. for 2 h. The mixture was filtered over CELITE®, the solids were washed with EtOAc and the filtrate was concentrated. MeOH was added, the mixture was filtered and the solids were dried, yielding the title compound 4-(2-butyl-1-oxo-1,2-dihydro-2,7-naphthyridin-4-yl)-2-fluorobenzaldehyde, E54-2, as a solid (139 mg). The filtrate was concentrated and the residue was purified by reverse phase chromatography on an Isco REDISEP® Gold HP C18 column (15.5 g) eluting with ACN (from 1% to 100%) in an aq. solution of TFA (0.1%), yielding title compound 4-(2-butyl-1-oxo-1,2-dihydro-2,7-naphthyridin-4-yl)-2-fluorobenzaldehyde, E54-2, as a solid (215 mg).

Method LCMS_MLG8: Rt=0.93 min; [M+H]⁺=325.

### Step 2: 1-(5-(4-((1-(4-(2-butyl-1-oxo-1,2-dihydro-2,7-naphthyridin-4-yl)-2-fluorobenzyl)piperidin-4-yl)oxy)piperidine-1-carbonyl)-2-methoxyphenyl)dihydropyrimidine-2,4(1H,3H)-dione (Compound E54)

To a solution of 4-(2-butyl-1-oxo-1,2-dihydro-2,7-naphthyridin-4-yl)-2-fluorobenzaldehyde (E54-2, 66 mg, 0.151 mmol), TEA (0.070 ml, 0.350 mmol), and 1-(2-methoxy-5-(4-(piperidin-4-yloxy)piperidine-1-carbonyl)phenyl)dihydropyrimidine-2,4(1H,3H)-dione HCl salt (Intermediate 29, 100 mg, 0.180 mmol) in DMSO (1.5 ml) under an argon atmosphere was added a solution of ZnCl₂ (0.5 M) in THF (0.350 ml, 0.175 mmol) and the RM was stirred at RT for 7 h. Solid NaBH₃CN (11 mg, 0.175 mmol) was added and the RM was stirred at 50° C. for 16 h. HOAc (0.050 ml, 0.873 mmol) was added and the RM was stirred at RT for 1 day. The mixture was purified by reverse phase chromatography on an Isco REDISEP® Gold HP C18 column (15.5 g) eluting with ACN (from 1% to 100%) in an aq. solution of N₁₄ HCO₃ (0.1%), followed by a purification by SFC on a Reprosphere PEI column (250×30 mm, 5 μm, 100 A) eluting with MeOH (from 20% to 28%) in supercritical CO₂, yielding the title compound 1-(5-(4-((1-(4-(2-butyl-1-oxo-1,2-dihydro-2,7-naphthyridin-4-yl)-2-fluorobenzyl)piperidin-4-yl)oxy)piperidine-1-carbonyl)-2-methoxyphenyl)dihydropyrimidine-2,4(1H,3H)-dione, Compound E54, as a solid (12.5 mg).

Method LCMS_MLG8: Rt=0.56 min; [M−H⁺]⁻=737.

¹H NMR (400 MHz, DMSO-d₆) δ[ppm] 10.32 (s, 1H), 9.44 (s, 1H), 8.72 (d, J=5.7 Hz, 1H), 7.87 (s, 1H), 7.54 (t, J=7.9 Hz, 1H), 7.47 (d, J=5.7 Hz, 1H), 7.38 (m, 1H), 7.34-7.27 (m, 3H), 7.15 (d, J=8.6 Hz, 1H), 4.03 (m, 3H), 3.84 (s, 3H), 3.69 (m, 1H), 3.64-3.55 (m, 4H), 3.47 (m, 1H), 3.21 (m, 3H), 2.75 (m, 2H), 2.68 (t, J=6.7 Hz, 2H), 2.21 (m, 2H), 1.81 (m, 4H), 1.75-1.66 (m, 2H), 1.45 (m, 4H), 1.33 (m, 2H), 0.92 (t, J=7.4 Hz, 3H).

### Compound E55: 1-(2-chloro-5-(4-(((3R,4R)-1-(4-(4,5-dimethyl-6-oxo-1-propyl-1,6-dihydropyridin-3-yl)-2,6-dimethoxyphenethyl)-3-fluoropiperidin-4-yl)oxy)piperidine-1-carbonyl)phenyl)dihydropyrimidine-2,4(1H,3H)-dione

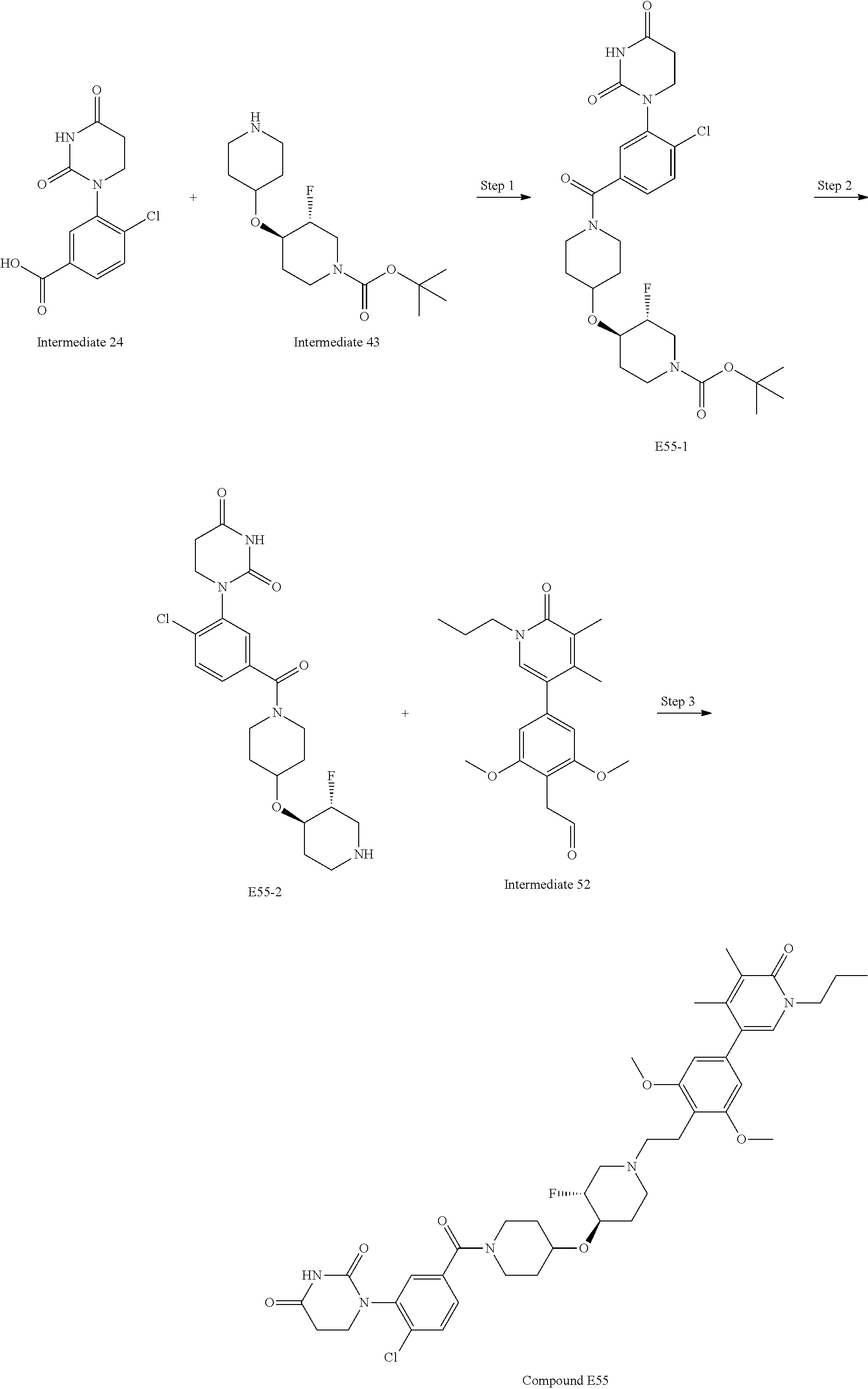 [SMILES: O=C1CCN(c2cc(C(=O)O)ccc2Cl)C(=O)N1 ; CC(C)(C)OC(=O)N1CC[C@@H](OC2CCNCC2)[C@H](F)C1 ; CC(C)(C)OC(=O)N1CC[C@@H](OC2CCN(C(=O)c3ccc(Cl)c(N4CCC(=O)NC4=O)c3)CC2)[C@H](F)C1 ; O=C1CCN(c2cc(C(=O)N3CCC(O[C@@H]4CCNC[C@H]4F)CC3)ccc2Cl)C(=O)N1 ; CCCn1cc(-c2cc(OC)c(CC=O)c(OC)c2)c(C)c(C)c1=O ; CCCn1cc(-c2cc(OC)c(CCN3CC[C@@H](OC4CCN(C(=O)c5ccc(Cl)c(N6CCC(=O)NC6=O)c5)CC4)[C@H](F)C3)c(OC)c2)c(C)c(C)c1=O]

### Step 1: tert-butyl (3R,4R)-4-((1-(4-chloro-3-(2,4-dioxotetrahydropyrimidin-1(2H)-yl)benzoyl)piperidin-4-yl)oxy)-3-fluoropiperidine-1-carboxylate (E55-1)

To a solution of 4-chloro-3-(2,4-dioxotetrahydropyrimidin-1(2H)-yl)benzoic acid (Intermediate 24,150 mg, 0.557 mmol) and DIEA (0.300 ml, 1.718 mmol) in DMF (4 ml) was added HATU (231 mg, 0.608 mmol) and the RM was stirred at RT for 30 min. Tert-butyl (3R,4R)-3-fluoro-4-(piperidin-4-yloxy)piperidine-1-carboxylate 4-methylbenzenesulfonate salt (Intermediate 43, 253 mg, 0.506 mmol) was added and the RM was stirred at RT for 4 h. The mixture was purified by reverse phase chromatography on a REDISEP® Gold HP C18 column (15.5 g) eluting with ACN (from 1% to 100%) in an aq. solution of TFA (0.1%), yielding the title compound tert-butyl (3R,4R)-4-((1-(4-chloro-3-(2,4-dioxotetrahydropyrimidin-1(2H)-yl)benzoyl)piperidin-4-yl)oxy)-3-fluoropiperidine-1-carboxylate, E55-1, as a solid (248 mg).

Method LCMS_MLG10: Rt=0.95 min; [M-Boc+H]⁺=453.

### Step 2: 1-(2-chloro-5-(4-(((3R,4R)-3-fluoropiperidin-4-yl)oxy)piperidine-1-carbonyl)phenyl)dihydropyrimidine-2,4(1H,3H)-dione (E55-2)

To a solution of tert-butyl (3R,4R)-4-((1-(4-chloro-3-(2,4-dioxotetrahydropyrimidin-1(2H)-yl)benzoyl)piperidin-4-yl)oxy)-3-fluoropiperidine-1-carboxylate (E55-1, 248 mg, 0.439 mmol) in DCM (3 ml) was added TFA (1 ml, 12.98 mmol) and the RM was stirred at RT for 1 h. The mixture was concentrated and the residue was purified by reverse phase chromatography on a REDISEP® Gold HP C18 column (15.5 g) eluting with ACN (from 1% to 100%) in an aq. solution of TFA (0.1%), yielding the title compound 1-(2-chloro-5-(4-(((3R,4R)-3-fluoropiperidin-4-yl)oxy)piperidine-1-carbonyl)phenyl)dihydropyrimidine-2,4(1H,3H)-dione, E55-2, as a solid TFA salt (211 mg).

Method LCMS_MLG3: Rt=0.58 min; [M+H]⁺=453.

### Step 3: 1-(2-chloro-5-(4-(((3R,4R)-1-(4-(4,5-dimethyl-6-oxo-1-propyl-1,6-dihydropyridin-3-yl)-2,6-dimethoxyphenethyl)-3-fluoropiperidin-4-yl)oxy)piperidine-1-carbonyl)phenyl)dihydropyrimidine-2,4(1H,3H)-dione (Compound E55)

A mixture of 1-(2-chloro-5-(4-(((3R,4R)-3-fluoropiperidin-4-yl)oxy)piperidine-1-carbonyl)phenyl)dihydropyrimidine-2,4(1H,3H)-dione TFA salt (E55-2, 162 mg, 0.286 mmol), HOAc (0.020 ml, 0.343 mmol) and NaOAc (31 mg, 0.378 mmol) in DCM (1.5 ml) was stirred at RT for 10 min. A solution of 2-(4-(4,5-dimethyl-6-oxo-1-propyl-1,6-dihydropyridin-3-yl)-2,6-dimethoxyphenyl)acetaldehyde (Intermediate 52, 108 mg, 0.314 mmol) in DMF (1.5 ml) was added and the RM was stirred at RT for 1.5 h. Solid NaBH(OAc)₃ (121 mg, 0.571 mmol) was added and the RM was stirred at RT for 20 h. The mixture was concentrated and the residue was purified by reverse phase chromatography on a REDISEP® Gold HP C18 column (15.5 g) eluting with ACN (from 0% to 100%) in an aq. solution of TFA (0.1%). Fractions containing the title compound were combined, concentrated and the residue was purified by reverse phase chromatography on a REDISEP® Gold HP C18 column (15.5 g) eluting with ACN (from 1% to 100%) in an aq. solution of NH₄HCO₃ (0.1%), yielding the title compound 1-(2-chloro-5-(4-(((3R,4R)-1-(4-(4,5-dimethyl-6-oxo-1-propyl-1,6-dihydropyridin-3-yl)-2,6-dimethoxyphenethyl)-3-fluoropiperidin-4-yl)oxy)piperidine-1-carbonyl)phenyl)dihydropyrimidine-2,4(1H,3H)-dione, Compound E55, as a solid (225 mg).

Method LCMS_MLG2: Rt=0.66 min; [M+H]⁺=780.

¹H NMR (600 MHz, DMSO-d₆) δ[ppm]10.50 (s, 1H), 7.64 (d, J=8.2 Hz, 1H), 7.57 (d, J=2.0 Hz, 1H), 7.45 (s, 1H), 7.40 (dd, J=8.2, 2.0 Hz, 1H), 6.50 (s, 2H), 4.36 (m, 1H), 3.92 (m, 1H), 3.85 (t, J=7.3 Hz, 2H), 3.77 (m, 8H), 3.68-3.58 (m, 1H), 3.49 (m, 2H), 3.14 (m, 3H), 2.81-2.68 (m, 5H), 2.43-2.33 (m, 3H), 2.15-2.03 (m, 7H), 1.98-1.73 (m, 3H), 1.66 (m, 2H), 1.54-1.36 (m, 3H), 0.86 (t, J=7.4 Hz, 3H).

### Compound E56: 1-(5-(4-(((3R,4R)-4-(4-(2-butyl-1-oxo-1,2-dihydro-2,7-naphthyridin-4-yl)-2,6-difluorophenoxy)-3-fluoropiperidin-1-yl)methyl)piperidine-1-carbonyl)-2-methoxyphenyl)dihydropyrimidine-2,4(1H,3H)-dione

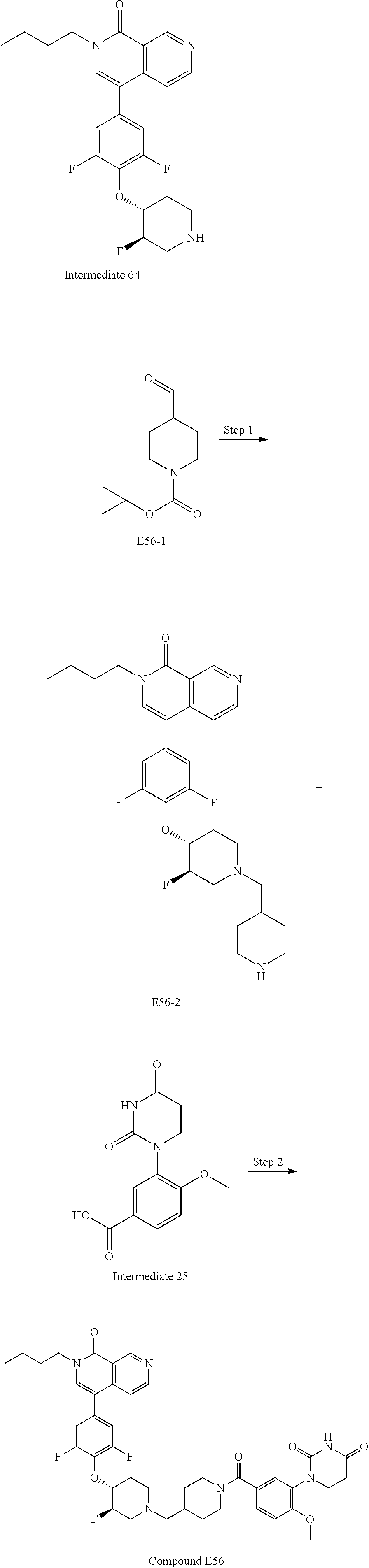 [SMILES: CCCCn1cc(-c2cc(F)c(O[C@@H]3CCNC[C@H]3F)c(F)c2)c2ccncc2c1=O ; CC(C)(C)OC(=O)N1CCC(C=O)CC1 ; CCCCn1cc(-c2cc(F)c(O[C@@H]3CCN(CC4CCNCC4)C[C@H]3F)c(F)c2)c2ccncc2c1=O ; COc1ccc(C(=O)O)cc1N1CCC(=O)NC1=O ; CCCCn1cc(-c2cc(F)c(O[C@@H]3CCN(CC4CCN(C(=O)c5ccc(OC)c(N6CCC(=O)NC6=O)c5)CC4)C[C@H]3F)c(F)c2)c2ccncc2c1=O]

### Step 1: 2-butyl-4-(3,5-difluoro-4-(((3R,4R)-3-fluoro-1-(piperidin-4-ylmethyl)piperidin-4-yl)oxy)phenyl)-2,7-naphthyridin-1(2H)-one (E56-2)

To a mixture of 2-butyl-4-(3,5-difluoro-4-(((3R,4R)-3-fluoropiperidin-4-yl)oxy)phenyl)-2,7-naphthyridin-1(2H)-one TFA salt (Intermediate 64, 218 mg, 0.160 mmol), tert-butyl 4-formylpiperidine-1-carboxylate (E56-1, 44 mg, 0.206 mmol) and TEA (0.075 ml, 0.538 mmol) in MeOH (4 ml) under an argon atmosphere was added a solution of ZnCl₂ (0.7 M) in THE (0.300 ml, 0.210 mmol) and the RM was stirred at RT for 7 h. Solid NaBH₃CN (18 mg, 0.286 mmol) was added and the RM was stirred at RT for 18 h. The mixture was concentrated and the residue was diluted with DCM (1.5 ml). TFA (0.350 ml, 4.54 mmol) was added and the RM was stirred at RT for 1 h. The mixture was concentrated and the residue was purified by reverse phase chromatography on a REDISEP® Gold HP C18 column (15.5 g) eluting with ACN (from 1% to 100%) in an aq. solution of TFA (0.1%), yielding the title compound 2-butyl-4-(3,5-difluoro-4-(((3R,4R)-3-fluoro-1-(piperidin-4-ylmethyl)piperidin-4-yl)oxy)phenyl)-2,7-naphthyridin-1(2H)-one, E56-2, as a solid TFA salt (136 mg).

Method LCMS_MLG9: Rt=0.55 min; [M+H]⁺=529.

### Step 2: 1-(5-(4-(((3R,4R)-4-(4-(2-butyl-1-oxo-1,2-dihydro-2,7-naphthyridin-4-yl)-2,6-difluorophenoxy)-3-fluoropiperidin-1-yl)methyl)piperidine-1-carbonyl)-2-methoxyphenyl)dihydropyrimidine-2,4(1H,3H)-dione (Compound E56)

To a solution of 3-(2,4-dioxotetrahydropyrimidin-1(2H)-yl)-4-methoxybenzoic acid (Intermediate 25, 18 mg, 0.068 mmol) in DMF (0.5 ml) were added DIEA (0.030 ml, 0.172 mmol) and HATU (27 mg, 0.071 mmol) and the RM was stirred at RT for 30 min. A solution of 2-butyl-4-(3,5-difluoro-4-(((3R,4R)-3-fluoro-1-(piperidin-4-ylmethyl)piperidin-4-yl)oxy)phenyl)-2,7-naphthyridin-1(2H)-one TFA salt (E56-2, 50 mg, 0.059 mmol) and DIEA (0.030 ml, 0.172 mmol) in DMF (0.5 ml) was added and the RM was stirred at RT for 2 h. The mixture was purified by reverse phase chromatography on a REDISEP® Gold HP C18 column (15.5 g) eluting with ACN (from 1% to 100%) in an aq. solution of TFA (0.1%). Fractions containing the title compound were combined, concentrated and the residue was purified by reverse phase chromatography on a REDISEP® Gold HP C18 column (15.5 g) eluting with ACN (from 1% to 100%) in an aq. solution of NH₄HCO₃ (0.1%), yielding the title compound 1-(5-(4-(((3R,4R)-4-(4-(2-butyl-1-oxo-1,2-dihydro-2,7-naphthyridin-4-yl)-2,6-difluorophenoxy)-3-fluoropiperidin-1-yl)methyl)piperidine-1-carbonyl)-2-methoxyphenyl)dihydropyrimidine-2,4(1H,3H)-dione, Compound E56, as a solid (26 mg).

Method LCMS_MLG9: Rt=0.65 min; [M+H]⁺=775.

¹H NMR (600 MHz, DMSO-d₆) δ[ppm]10.33 (s, 1H), 9.44 (s, 1H), 8.74 (d, J=5.6 Hz, 1H), 7.88 (s, 1H), 7.49 (d, J=5.6 Hz, 1H), 7.39-7.29 (m, 4H), 7.16 (d, J=8.6 Hz, 1H), 4.70 (m, 1H), 4.40 (m, 1H), 4.21 (m, 1H), 4.02 (t, J=7.4 Hz, 2H), 3.85 (m, 4H), 3.60 (t, J=6.6 Hz, 2H), 3.11 (m, 3H), 2.77 (m, 1H), 2.69 (m, 2H), 2.24 (m, 2H), 2.16 (m, 1H), 2.09 (m, 2H), 1.84-1.67 (m, 6H), 1.33 (m, 2H), 1.06 (m, 2H), 0.92 (t, J=7.4 Hz, 3H).

TABLE 1
summarizes the synthesis of exemplary compounds using methods analogous to the
examples described above.

| Cmpd | | | | Synthetic |
|---|---|---|---|---|
| No. | Structure | Chemical name | Analytical data | Scheme |
| E4 | 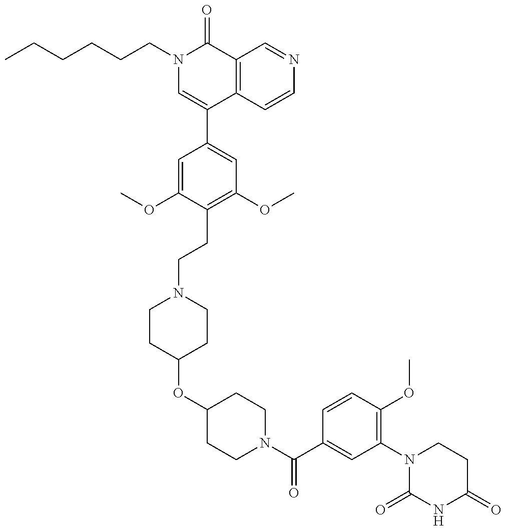 [SMILES: CCCCCCn1cc(-c2cc(OC)c(CCN3CCC(OC4CCN(C(=O)c5ccc(OC)c(N6CCC(=O)NC6=O)c5)CC4)CC3)c(OC)c2)c2ccncc2c1=O] | 1-(5-(4-((1-(4-(2-hexyl- 1-oxo-1,2-dihydro-2,7- naphthyridin-4-yl)-2,6- dimethoxy- phenethyl)piperidin-4- yl)oxy)piperidine-1- carbonyl)-2-meth- oxyphenyl)dihydro- pyrimidine-2,4(1H,3H)- dione | Method LCMS_MLG8: Rt = 0.79 min; [M + H]⁺ = 823. ¹H NMR (400 MHz, DMSO-d₆) δ [ppm] 10.33 (s, 1H), 9.43 (s, 1H), 8.70 (d, J = 5.6 Hz, 1H), 7.82 (s, 1H), 7.55 (d, J = 5.7 Hz, 1H), 7.41-7.29 (m, 2H), 7.15 (d, J = 8.5 Hz, 1H), 6.68 (s, 2H), 4.02 (m, 2H), 3.82 (m, 9H), 3.78- 3.53 (m, 5H), 3.44 (m, 1H), 3.20 (m, 2H), 2.84-2.63 (m, 6H), 2.34 (m, 2H), 2.20-2.04 (m, 2H), 1.77 (m, 6H), 1.48- 1.25 (m, 10H), 0.85 (m, 3H). | Analogous to the synthesis of Compound E15 |
| E13 | 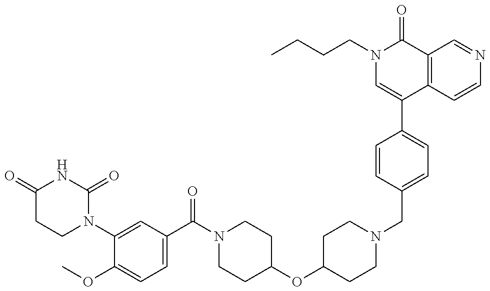 [SMILES: CCCCn1cc(-c2ccc(CN3CCC(OC4CCN(C(=O)c5ccc(OC)c(N6CCC(=O)NC6=O)c5)CC4)CC3)cc2)c2ccncc2c1=O] | 1-(5-(4-((1-(4-(2-butyl- 1-oxo-1,2-dihydro-2,7- naphthyridin-4- yl)benzyl)piperidin-4- yl)oxy)piperidine-1- carbonyl)-2-meth- oxyphenyl)dihydro- pyrimidine-2,4(1H,3H)- dione | Method LCMS_MLG8: Rt = 0.57 min; [M + H]⁺ = 721. ¹H NMR (600 MHz, DMSO-d₆) δ [ppm] 10.33 (s, 1H), 9.44 (s, 1H), 8.71 (d, J = 5.6 Hz, 1H), 7.80 (s, 1H), 7.48-7.30 (m, 7H), 7.15 (d, J = 8.5 Hz, 1H), 4.06 (m, 3H), 3.84 (m, 4H), 3.69 (m, 1H), 3.59 (t, J = 6.7 Hz, 2H), 3.53 (s, 2H), 3.51-3.44 (m, 1H), 3.21 (m, 2H), 2.70 (m, 4H), | Analogous to the synthesis of Compound E54 |
| | | | 2.14 (m, 2H), 1.88- | |
| | | | 1.76 (m, 4H), 1.76- | |
| | | | 1.63 (m, 2H), 1.39 | |
| | | | (m, 6H), 0.91 (t, J = | |
| | | | 7.4 Hz, 3H). | |
| E14 | 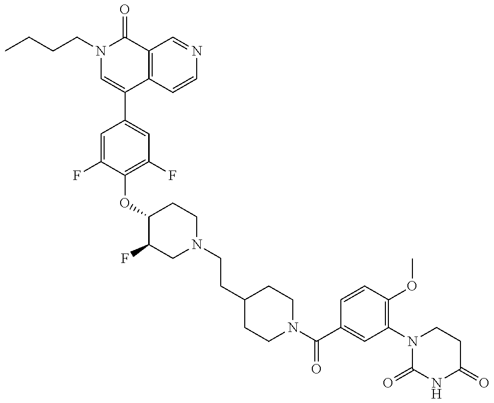 [SMILES: CCCCn1cc(-c2cc(F)c(O[C@@H]3CCN(CCC4CCN(C(=O)c5ccc(OC)c(N6CCC(=O)NC6=O)c5)CC4)C[C@H]3F)c(F)c2)c2ccncc2c1=O] | 1-(5-(4-(2-((3R,4R)-4- (4-(2-butyl-1-oxo-1,2- dihydro-2,7- naphthyridin-4-yl)-2,6- difluorophenoxy)-3- fluoropiperidin-1- yl)ethyl)piperidine-1- carbonyl)-2-meth- oxyphenyl)dihydro- pyrimidine-2,4(1H,3H)- dione | Method LCMS_MLG9: Rt = 0.66 min; [M + H]⁺ = 789. ¹H NMR (600 MHz, DMSO-d₆) δ [ppm] 10.33 (s, 1H), 9.43 (d, J = 0.8 Hz, 1H), 8.73 (d, J = 5.6 Hz, 1H), 7.88 (s, 1H), 7.50 (dd, J = 5.7, 0.9 Hz, 1H), 7.36 (dd, J = 8.5, 2.2 Hz, 1H), 7.32 (m, 3H), 7.15 (d, J = 8.6 Hz, 1H), 4.39 (m, 1H), 4.21 (m, 1H), 4.02 (t, J = 7.4 Hz, 2H), 3.84 (m, 4H), 3.60 (t, J = 6.7 Hz, 2H), 2.69 (m, 6H), 2.32 (t, J = 7.4 Hz, 2H), 2.15 (m, 2H), 1.93 (m, 2H), 1.82-1.51 (m, 7H), 1.40 (m, 2H), | Analogous to the synthesis of Compound E6 |
| | | | 1.33 (m, 2H), 1.15- | |
| | | | 1.05 (m, 2H), 0.92 | |
| | | | (t, J = 7.4 Hz, 3H). | |
| E16 | 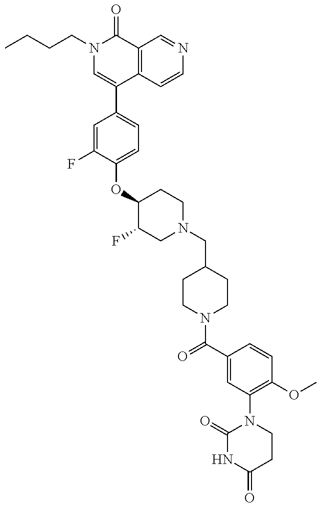 [SMILES: CCCCn1cc(-c2ccc(O[C@H]3CCN(CC4CCN(C(=O)c5ccc(OC)c(N6CCC(=O)NC6=O)c5)CC4)C[C@@H]3F)c(F)c2)c2ccncc2c1=O] | 1-(5-(4-(((3S,4S)-4-(4- (2-butyl-1-oxo-1,2- dihydro-2,7- naphthyridin-4-yl)-2- fluorophenoxy)-3- fluoropiperidin-1- yl)methyl)piperidine-1- carbonyl)-2-meth- oxyphenyl)dihydro- pyrimidine-2,4(1H,3H)- dione | Method LCMS_MLG2: Rt = 0.73 min; [M + H]⁺ = 757. ¹H NMR (600 MHz, DMSO-d₆) δ [ppm] 10.33 (s, 1H), 9.43 (s, 1H), 8.72 (d, J = 5.6 Hz, 1H), 7.81 (s, 1H), 7.44 (m, 2H), 7.37 (m, 2H), 7.32 (m, 1H), 7.22 (m, 1H), 7.16 (m, 1H), 4.68 (m, 1H), 4.53 (m, 1H), 4.02 (m, 2H), 3.85 (s, 3H), 3.60 (m, 2H), 3.29 (m, 1H), 3.12 (m, 1H), 2.68 (m, 4H), 2.20 (m, 6H), 1.70 (m, 7H), 1.36-1.31 (m, 2H), 1.07 (m, 2H), 0.92 (t, J = 7.4 Hz, 3H). | Analogous to the synthesis of Compound E34 |
| E18 | 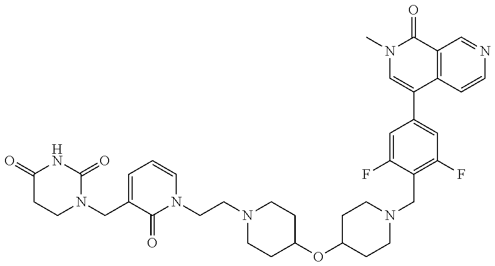 [SMILES: Cn1cc(-c2cc(F)c(CN3CCC(OC4CCN(CCn5cccc(CN6CCC(=O)NC6=O)c5=O)CC4)CC3)c(F)c2)c2ccncc2c1=O] | 1-((1-(2-(4-((1-(2,6- difluoro-4-(2-methyl-1- oxo-1,2-dihydro-2,7- naphthyridin-4- yl)benzyl)piperidin-4- yl)oxy)piperidin-1- yl)ethyl)-2-oxo-1,2- dihydropyridin-3- yl)methyl)dihydro- pyrimidine-2,4(1H,3H)- dione | Method LCMS_MLG9: Rt = 0.39 min; [M + H]⁺ = 716. ¹H NMR (600 MHz, DMSO-d₆) δ [ppm] 10.14 (s, 1H), 9.44 (d, J = 0.8 Hz, 1H), 8.74 (d, J = 5.7 Hz, 1H), 7.94 (s, 1H), 7.57 (m, 1H), 7.54- 7.50 (m, 1H), 7.26 (m, 3H), 6.19 (m, 1H), 4.26 (s, 2H), 3.98 (t, J = 6.4 Hz, 2H), 3.59 (m, 5H), | Analogous to the synthesis of Compound E24 |
| | | | 3.47-3.39 (m, 4H), | |
| | | | 2.71 (m, 4H), 2.62- | |
| | | | 2.56 (m, 4H), 2.17 | |
| | | | (m, 2H), 2.09 (m, | |
| | | | 2H), 1.74 (m, 4H), | |
| | | | 1.36 (m, 4H). | |
| E30 | 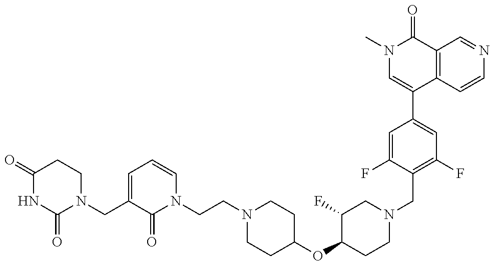 [SMILES: Cn1cc(-c2cc(F)c(CN3CC[C@@H](OC4CCN(CCn5cccc(CN6CCC(=O)NC6=O)c5=O)CC4)[C@H](F)C3)c(F)c2)c2ccncc2c1=O] | 1-((1-(2-(4-(((3R,4R)-1- (2,6-difluoro-4-(2- methyl-1-oxo-1,2- dihydro-2,7- naphthyridin-4- yl)benzyl)-3- fluoropiperidin-4- yl)oxy)piperidin-1- yl)ethyl)-2-oxo-1,2- dihydropyridin-3- yl)methyl)dihydro- pyrimidine-2,4(1H,3H)- dione | Method LCMS_MLG2: Rt = 0.43 min; [M + H]⁺ = 734. ¹H NMR (600 MHz, DMSO-d₆) δ [ppm] 10.14 (s, 1H), 9.44 (d, J = 0.9 Hz, 1H), 8.74 (d, J = 5.7 Hz, 1H), 7.95 (s, 1H), 7.57 (dd, J = 6.8, 2.0 Hz, 1H), 7.54 (dd, J = 5.7, 0.9 Hz, 1H), 7.27 (m, 3H), 6.19 (t, J = 6.8 Hz, 1H), 4.40-4.27 (m, 1H), | Analogous to the synthesis of Compound E39 |
| | | | 4.26 (s, 2H), 3.98 (t, | |
| | | | J = 6.5 Hz, 2H), 3.70 | |
| | | | (s, 2H), 3.58 (s, 3H), | |
| | | | 3.47-3.39 (m, 4H), | |
| | | | 3.03 (m, 1H), 2.71 | |
| | | | (m, 3H), 2.59-2.53 | |
| | | | (m, 4H), 2.26-2.04 | |
| | | | (m, 4H), 1.90 (m, | |
| | | | 1H), 1.75 (m, 2H), | |
| | | | 1.43-1.33 (m, 3H). | |
| E31 | 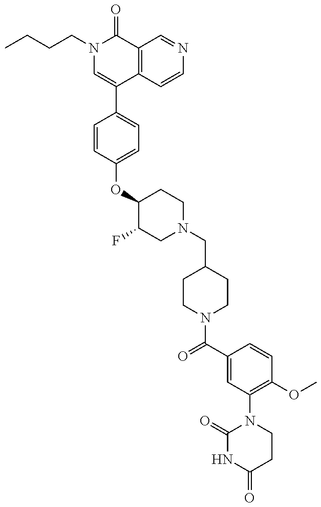 [SMILES: CCCCn1cc(-c2ccc(O[C@H]3CCN(CC4CCN(C(=O)c5ccc(OC)c(N6CCC(=O)NC6=O)c5)CC4)C[C@@H]3F)cc2)c2ccncc2c1=O] | 1-(5-(4-(((3S,4S)-4-(4- (2-butyl-1-oxo-1,2- dihydro-2,7- naphthyridin-4- yl)phenoxy)-3- fluoropiperidin-1- yl)methyl)piperidine-1- carbonyl)-2-meth- oxyphenyl)dihydro- pyrimidine-2,4(1H,3H)- dione | Method LCMS_MLG2: Rt = 0.68 min; [M + H]⁺ = 739. ¹H NMR (400 MHz, DMSO-d₆) δ [ppm] 10.33 (s, 1H), 9.43 (d, J = 0.8 Hz, 1H), 8.70 (d, J = 5.7 Hz, 1H), 7.74 (s, 1H), 7.44-7.35 (m, 4H), 7.32 (d, J = 2.1 Hz, 1H), 7.19-7.13 (m, 3H), 4.66 (m, 1H), 4.57-4.21 (m, 2H), 4.03 (t, J = 7.4 Hz, 2H), 3.85 (m, 4H), 3.60 (t, J = 6.7 Hz, 2H), 3.12 (m, 1H), 3.07-2.65 (m, 6H), 2.22 (m, 5H), 1.82 (m, 1H), 1.71 (m, 3H), 1.58 (m, 1H), 1.33 (m, 2H), 1.07 (m, 2H), 0.92 (t, J = 7.4 Hz, 3H). | Analogous to the synthesis of Compound E32 |
| E33 | 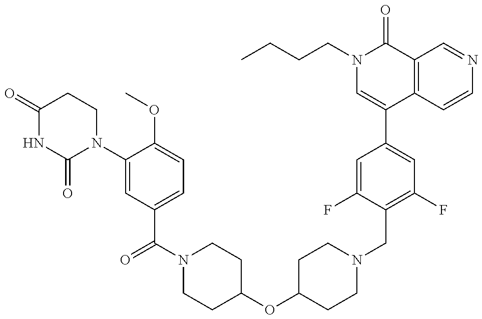 [SMILES: CCCCn1cc(-c2cc(F)c(CN3CCC(OC4CCN(C(=O)c5ccc(OC)c(N6CCC(=O)NC6=O)c5)CC4)CC3)c(F)c2)c2ccncc2c1=O] | 1-(5-(4-((1-(4-(2-butyl- 1-oxo-1,2-dihydro-2,7- naphthyridin-4-yl)-2,6- difluorobenzyl)piperi- din-4-yl)oxy)piperi- dine-1-carbonyl)-2- methoxyphenyl)di- hydro-pyrimidine- 2,4(1H,3H)-dione | Method LCMS_MLG1: Rt = 0.68 min; [M + H]⁺ = 757. ¹H NMR (400 MHz, DMSO-d₆) δ [ppm] 10.32 (s, 1H), 9.44 (s, 1H), 8.74 (d, J = 5.7 Hz, 1H), 7.93 (s, 1H), 7.52 (d, J = 5.7 Hz, 1H), 7.37 (dd, J = 8.4, 2.2 Hz, 1H), 7.33 (d, J = 2.1 Hz, 1H), 7.26 (d, J = 8.0 Hz, 2H), 7.14 (d, J = 8.5 Hz, 1H), 4.03 (t, J = 7.4 Hz, 2H), 3.84 (s, 3H), 3.68 (m, 1H), 3.63-3.56 (m, 4H), 3.43 (m, 1H), | Analogous to the synthesis of Compound E54 |
| | | | 3.25-3.14 (m, 4H), | |
| | | | 2.70 (m, 4H), 2.19 | |
| | | | (m, 2H), 1.89-1.65 | |
| | | | (m, 6H), 1.38 (m, | |
| | | | 6H), 0.92 (t, J = 7.3 | |
| | | | Hz, 3H). | |
| E47 | 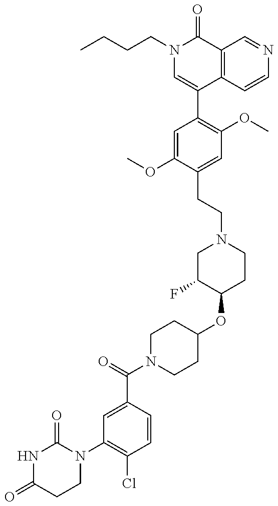 [SMILES: CCCCn1cc(-c2cc(OC)c(CCN3CC[C@@H](OC4CCN(C(=O)c5ccc(Cl)c(N6CCC(=O)NC6=O)c5)CC4)[C@H](F)C3)cc2OC)c2ccncc2c1=O] | 1-(5-(4-(((3R,4R)-1-(4- (2-butyl-1-oxo-1,2- dihydro-2,7- naphthyridin-4-yl)-2,5- dimethoxyphenethyl)-3- fluoropiperidin-4- yl)oxy)piperidine-1- carbonyl)-2- chlorophenyl)dihydro- pyrimidine-2,4(1H,3H)- dione | Method LCMS_MLG-new- 2: Rt = 0.78 min; [M + H]⁺ = 817. ¹H NMR (600 MHz, DMSO-d₆) δ [ppm] 10.51 (s, 1H), 9.40 (s, 1H), 8.63 (d, J = 5.6 Hz, 1H), 7.70 (s, 1H), 7.64 (d, J = 8.2 Hz, 1H), 7.58 (d, J = 2.1 Hz, 1H), 7.41 (dd, J = 8.2, 2.0 Hz, 1H), 7.06-7.00 (m, 2H), 6.88 (s, 1H), 4.46-4.30 (m, 1H), 4.00 (m, 3H), 3.75 (m, 5H), 3.63 (m, 4H), 3.51 (m, 2H), 3.29-3.08 (m, 3H), 2.85-2.69 (m, 5H), 2.59 (m, 2H), 2.21 (m, 1H), 2.14 (m, 1H), 1.97 (m, 1H), 1.84 (m, 2H), 1.70 (m, 2H), 1.55-1.38 (m, 3H), 1.34 (m, 2H), 0.92 (t, J = 7.4 Hz, 3H). | Analogous to the synthesis of Compound E55 |
| E50 | 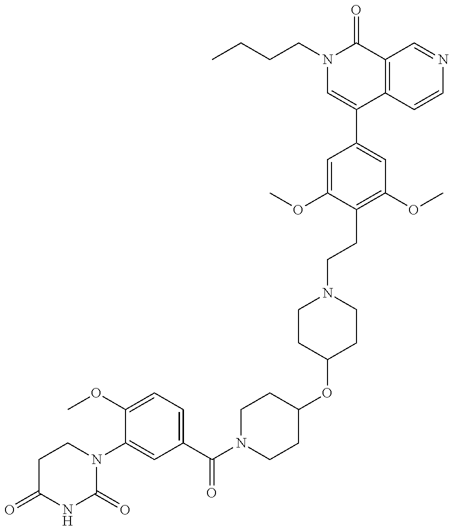 [SMILES: CCCCn1cc(-c2cc(OC)c(CCN3CCC(OC4CCN(C(=O)c5ccc(OC)c(N6CCC(=O)NC6=O)c5)CC4)CC3)c(OC)c2)c2ccncc2c1=O] | 1-(5-(4-((1-(4-(2-butyl- 1-oxo-1,2-dihydro-2,7- naphthyridin-4-yl)-2,6- dimethoxyphenethyl)- piperidin-4- yl)oxy)piperidine-1- carbonyl)-2-meth- oxyphenyl)dihydro- pyrimidine-2,4(1H,3H)- dione | Method LCMS_MLG4: Rt = 2.87 min; [M + H]⁺ = 795. ¹H NMR (400 MHz, DMSO-d₆) δ [ppm] 10.33 (s, 1H), 9.44 (d, J = 0.8 Hz, 1H), 8.71 (d, J = 5.6 Hz, 1H), 7.82 (s, 1H), 7.54 (dd, J = 5.7, 0.9 Hz, 1H), 7.38 (dd, J = 8.4, 2.2 Hz, 1H), 7.34 (d, J = 2.1 Hz, 1H), 7.15 (d, J = 8.6 Hz, 1H), 6.72 (s, 2H), 4.09 (m, 1H), 4.04 (t, J = 7.3 Hz, 2H), 3.84 (m, 9H), 3.71 (m, 2H), 3.60 (t, J = 6.7 Hz, 2H), 3.27-3.15 (m, 5H), 2.86 (m, 6H), 2.68 (t, J = 6.9 Hz, 2H), 1.96-1.77 (m, 4H), 1.76-1.66 (m, 2H), 1.65-1.38 (m, 4H), 1.34 (m, 2H), 0.93 (t, J =7.4 Hz, 3H). | Analogous to the synthesis of Compound E12 |
| E51 | 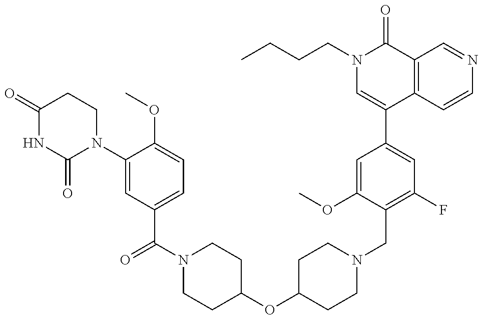 [SMILES: CCCCn1cc(-c2cc(F)c(CN3CCC(OC4CCN(C(=O)c5ccc(OC)c(N6CCC(=O)NC6=O)c5)CC4)CC3)c(OC)c2)c2ccncc2c1=O] | 1-(5-(4-((1-(4-(2-butyl- 1-oxo-1,2-dihydro-2,7- naphthyridin-4-yl)-2- fluoro-6- methoxybenzyl)- piperidin-4- yl)oxy)piperidine-1- carbonyl)-2-meth- oxyphenyl)dihydro- pyrimidine- 2,4(1H,3H)-dione | Method LCMS_MLG1: Rt = 0.71 min; [M + H]⁺ = 769. ¹H NMR (400 MHz, DMSO-d₆) δ [ppm] 10.32 (s, 1H), 9.45 (s, 1H), 8.73 (d, J = 5.7 Hz, 1H), 7.89 (s, 1H), 7.55 (d, J = 5.7 Hz, 1H), 7.37 (dd, J = 8.4, 2.2 Hz, 1H), 7.33 (d, J = 2.1 Hz, 1H), 7.15 (d, J = 8.5 Hz, 1H), 7.09-6.85 (m, 2H), 4.12-3.99 (m, 3H), 3.86 (m, 7H), 3.68 (m, 2H), 3.59 (m, 4H), 3.26- 3.13 (m, 4H), 2.68 | Analogous to the synthesis of Compound E64 |
| | | | (m, 3H), 2.18 (br. s, | |
| | | | 1H), 1.94-1.64 (m, | |
| | | | 6H), 1.54-1.28 (m, | |
| | | | 6H), 0.92 (t, J = 7.3 | |
| | | | Hz, 3H). | |
| E53 | 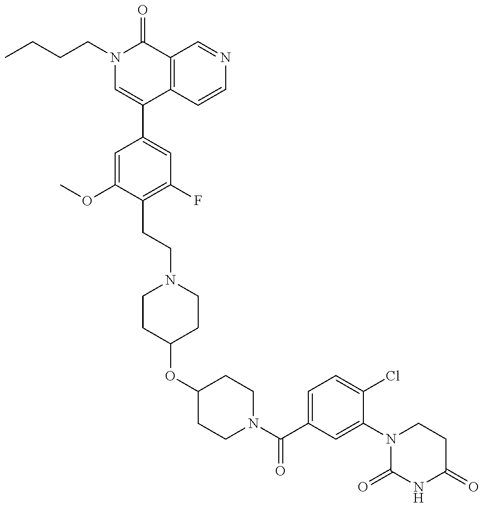 [SMILES: CCCCn1cc(-c2cc(F)c(CCN3CCC(OC4CCN(C(=O)c5ccc(Cl)c(N6CCC(=O)NC6=O)c5)CC4)CC3)c(OC)c2)c2ccncc2c1=O] | 1-(5-(4-((1-(4-(2-butyl- 1-oxo-1,2-dihydro-2,7- naphthyridin-4-yl)-2- fluoro-6- methoxyphenethyl)- piperidin-4- yl)oxy)piperidine-1- carbonyl)-2- chlorophenyl)dihydro- pyrimidine- 2,4(1H,3H)-dione | Method LCMS_MLG1: Rt = 0.78 min; [M − H]⁻ = 785. ¹H NMR (400 MHz, DMSO-d6) δ [ppm] 10.51 (s, 1H), 9.43 (s, 1H), 8.71 (d, J = 5.7 Hz, 1H), 7.85 (s, 1H), 7.63 (d, J = 8.2 Hz, 1H), 7.57 (s, 1H), 7.52 (d, J = 5.7 Hz, 1H), 7.40 (d, J = 8.2 Hz, 1H), 6.90 (d, J = 10.2 Hz, 2H), 4.03 (m, 3H), 3.86 (s, 3H), 3.79-3.44 (m, 5H), 3.21 (m, 2H), 2.75 (m, 6H), 2.28 (m, 4H), 1.87-1.63 (m, 6H), 1.49-1.26 (m, 6H), 0.92 (t, J = 7.3 Hz, 3H). | Analogous to the synthesis of Compound E52 |

### Example 3. Biological Data from BRD9 Protein Degradation Bioassays

Abbreviations for Biological Assay Description:
Amax Maximal Dose response
BRD Bromodomain
CHYSEL cis-acting hydrolase element
DC₅₀ degradation Constant
Deg degradation
DEST destination
DMEM Dulbecco's modified eagle's medium
FCS fetal calf serum
FSC forward side scatter
GFP green fluorescence protein
HEK human embryonic kidney
Ires internal ribosome entry site
Lenti lentiviral
μl microliter
NaPyr sodium pyruvate
NEAA non essential amino acids
ng nanogram
ORF open reading frame
PBS phosphate-buffered saline
SSC side side scatter
ul microliter
w/o without

Assay Description:

Compounds of the invention were tested in the following cellular assays. The data obtained is shown in Table 1. The terms in Table 1 are defined as follows: DC50 refers to the concentration at which 50% maximal degradation was observed; deg Amax is the extent of degradation and the value refers to the % protein remaining at the concentration at which maximum degradation is seen.

BRD9-GFP protein abundance flow cytometry assay in HEK293A cells: Degradation of BRD9 was measured in HEK293A cells expressing BRD9-GFP and mCherry from a stably integrated second generation bicistronic BRD9-GFP-CHYSELmCherry construct. Reduction of the GFP signal measured by flow cytometry served as readout for BRD9 degradation after degrader treatment.

i) Cloning of the pLenti6-BRD9-GFP-CHYSEL-mCherry Sensor Vector

The BRD9 protein abundance sensor is based on a second generation bicistronic construct, where the 2 reading frames BRD9 and mCherry control are separated by a cis acting hydrolase element (see: Lo et al., 2015 Cell Reports 13, 2634), replacing the Ires from a first generation vector.

The bicistronic BRD9-GFP-CHYSEL-mCherry construct is based on a pLenti6-DEST vector backbone where a GFP-CHYSEL-mCherry cassette was synthesized and inserted downstream of the DEST cassette by Gibson assembly resulting in the new gateway compatible vector pLenti6-DEST-GFP-CHYSEL-mCherry to allow Gateway cloning with pENTR221-BRD9 (w/o ATG) to obtain the final sensor construct pLenti6-BRD9-GFP-CHYSEL-mCherry.

Sequence of the synthesized construct (Xhol sites are shown in bold, mCherry-ORF is shown 30 in small letters):

(SEQ ID NO: 1)
GATATCCAGCACAGTGGCGGCCGCTCGAGcATGGTGAGCAAGGGCGAGGA
GCTGTTCACCGGGGTGGTGCCCATCCTGGTCGAGCTGGACGGCGACGTAA
ACGGCCACAAGTTCAGCGTGTCCGGCGAGGGCGAGGGCGATGCCACCTAC
GGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCC
GCTACCCCGACCACATGAAGCAGCACGACTTCTTCAAGTCCGCCATGCCC
GAAGGCTACGTCCAGGAGCGCACCATCTTCTTCAAGGACGACGGCAACTA
CAAGACCCGCGCCGAGGTGAAGTTCGAGGGCGACACCCTGGTGAACCGCA
TCGAGCTGAAGGGCATCGACTTCAAGGAGGACGGCAACATCCTGGGGCAC
AAGCTGGAGTACAACTACAACAGCCACAACGTCTATATCATGGCCGACAA
GCAGAAGAACGGCATCAAGGTGAACTTCAAGATCCGCCACAACATCGAGG
ACGGCAGCGTGCAGCTCGCCGACCACTACCAGCAGAACACCCCCATCGGC
GACGGCCCCGTGCTGCTGCCCGACAACCACTACCTGAGCACCCAGTCCGC
CCTGAGCAAAGACCCCAACGAGAAGCGCGATCACATGGTCCTGCTGGAGT
TCGTGACCGCCGCCGGGATCACTCTCGGCATGGACGAGCTGTACAAGGGA
AGCGGAGCGACGAATTTTAGTCTACTGAAACAAGCGGGAGACGTGGAGGA
AAACCCTGGACCTatggtgagcaagggcgaggaggataacatggccatca
tcaaggagttcatgcgcttcaaggtgcacatggagggctccgtgaacggc
cacgagttcgagatcgagggcgagggcgagggccgcccctacgagggcac
ccagaccgccaagctgaaggtgaccaagggtggccccctgcccttcgcct
gggacatcctgtcccctcagttcatgtacggctccaaggcctacgtgaag
caccccgccgacatccccgactacttgaagctgtccttccccgagggctt
caagtgggagcgcgtgatgaacttcgaggacggcggcgtggtgaccgtga
cccaggactcctccctgcaggacggcgagttcatctacaaggtgaagagc
gcggcaccaacttcccctccgacggccccgtaatgcagaagaagaccatg
ggctgggaggcctcctccgagcggatgtaccccgaggacggcgccctgaa
gggcgagatcaagcagaggctgaagctgaaggacggcggccactacgacg
ctgaggtcaagaccacctacaaggccaagaagcccgtgcagctgcccggc
gcctacaacgtcaacatcaagttggacatcacctcccacaacgaggacta
caccatcgtggaacagtacgaacgcgccgagggccgccactccaccggcg
gcatggacgagagtacaagtagCTCGAGTCTAGAGGGCCCGCGGTTAAC

ii) Engineering Stably Expressing HEK293 Å-BRD9-GFP-CHYSEL-mCherry Sensor Cells

HEK293 Å-BRD9-GFP-CHYSEL-mCherry sensor cells were generated by lentiviral vector transduction using the pLenti6-BRD9-GFP-CHYSEL-mCherry sensor construct described before. Lentiviral particles were produced in HEK293FT cells (Invitrogen R70007) by co-transfection of 500 ng pLenti6-BRD9-GFP-CHYSEL-mCherry, 500 ng delta8.71 and 200 ng pVSVG diluted in 100 μl OptiMEM serum free medium (Invitrogen #11058-021) that was mixed after 5 min preincubation with 3 μl of Lipofectamine2000 (Invitrogen #11668-019) in 97 μl OptiMEM serum free medium. The mix was incubated for another 20 min at RT and then added to 1 ml of a freshly prepared suspension of HEK293FT cells in a well of a 6-well plate (concentration 1.2×10⁶ cells/ml). 1 day after transfection, the medium was replaced with 1.5 ml of complete growth medium (DMEM high Glucose+10% FCS+1% L-Glutamine+1% NEAA+1% NaPyr.). 48 h post transfection supernatant containing viral transducing particles was collected and frozen at −80° C. Two days before transduction with viral particles 1×105 HEK293A cells (Invitrogen R70507) were seeded in 2 ml growth medium in a well of a 6-well plate. Infection was performed with 90 of collected supernatant containing viral transducing particles in 1 ml medium including 8 μg/ml polybrene. 24 h post infection, stably transfected cells were selected with blasticidin at a concentration of 8 μg/ml.

iii) Quantitative BRD9-GFP Abundance Measurements

Stable HEK293 Å-BRD9-GFP-CHYSEL-mCherry cells were maintained in complete growth medium (DMEM high Glucose+10% FCS+1% L-Glutamine+1% NEAA+1% NaPyr.) with passaging performed twice per week. On Day 0, HEK293 Å-BRD9-GFP-CHYSEL-mCherry cells were seeded at 10,000 cells/well in a 96-well microtiter plate in 260 complete medium. On Day 1, cells were treated in duplicate with 10-point 1:3 dilution series of compound using the HP D300 Digital Dispenser (Tecan). DMSO concentrations were normalized across the plate to 0.1%. On Day 2, after 24 h of incubation at 37° C., treatment media was discarded, cells rinsed with 100 ul/well PBS and then detached using 40 ul trypsin/well for 5 min. Trypsin was neutralized with 100 ul/well PBS+20% FCS). Flow cytometry was performed on the samples using the Beckman Coulter CytoFLEX flow cytometer. Cell identification was then performed using forward (FSC) vs. side scatter (SSC) plots. Single cell discrimination is performed using FSC-Width (FSC-W) vs. FSC-Height (FSC-H) plots. Median GFP/mCherry ratio values for 5,000 single cells are used to determine BRD9 levels. Median GFP/mCherry ratio values from HEK293 Å-mCherry are used as a background signal and thus defining 0% BRD9 signal. Median GFP/mCherry ratio values from DMSO treated HEK293 Å-BRD9-GFP-CHYSEL-mCherry are used to define 100% BRD9 signal for subsequent DC50 curves (concentration at 50% BRD9 degradation). Concentration response curves plotting relative reduction of the GFP/mCherry ratio signal (measured by flow cytometry) versus 10 compound concentrations (starting concentration 10 M, 3 fold dilution steps) of the compounds allowed generation of DC50 values. Data is shown in Tables 2 and 3, in which, DC50 refers to the concentration at which 50% maximal degradation was observed; deg Amax is the extent of degradation and the value refers to the % protein remaining at the concentration at which maximum degradation is seen.

Table 2 summarizes in vitro assay data related to BRD9 degradation in the presence of exemplary compounds.

| | BRD9 | BRD9 |
|---|---|---|
| Compound No | DC50 (μM) | Deg Amax % |
| A1 | 0.054 | 10.72 |
| A2 | 0.063 | 13.39 |
| A3 | 0.068 | 12.29 |
| A4 | 0.004 | 14.01 |
| A5 | 0.019 | 18.64 |
| A6 | 0.003 | 12.25 |
| A7 | 0.060 | 21.30 |
| A8 | 0.070 | 16.25 |
| A9 | 0.002 | 20.62 |
| A10 | 0.002 | 18.93 |
| A11 | 0.012 | 19.45 |
| A12 | 0.002 | 13.46 |
| A13 | 0.015 | 18.05 |
| A14 | 0.003 | 33.06 |
| A15 | 0.049 | 29.47 |
| A16 | 0.032 | 6.37 |
| A17 | 0.011 | 32.92 |
| A18 | 0.015 | 11.83 |
| A19 | 0.003 | 17.02 |
| A20 | 0.014 | 9.75 |
| A21 | 0.001 | 12.12 |
| A22 | 0.006 | 11.18 |
| A23 | 0.016 | 10.64 |
| A24 | 0.027 | 13.31 |
| A25 | 0.010 | 18.22 |
| A26 | 0.110 | 14.36 |
| A27 | 0.049 | 12.02 |
| A28 | 0.002 | 22.70 |
| A29 | 0.034 | 15.75 |
| A30 | 0.016 | 13.99 |
| A31 | 0.010 | 15.11 |
| A32 | 0.006 | 16.20 |
| A33 | 0.034 | 13.30 |
| A34 | 0.037 | 13.80 |
| A35 | 0.031 | 13.19 |
| A36 | 0.039 | 18.42 |
| A37 | 0.002 | 13.01 |
| A38 | 0.001 | 15.53 |
| A39 | 0.021 | 16.72 |
| A40 | 0.004 | 14.20 |
| A41 | 0.060 | 13.54 |
| A42 | 0.098 | 13.53 |
| B1 | 0.002 | 11.90 |
| B2 | 0.002 | 13.73 |
| B3 | 0.027 | 10.95 |
| B4 | 0.003 | 14.82 |
| B5 | 0.004 | 13.14 |
| B6 | 0.001 | 16.75 |
| B7 | 0.007 | 17.54 |
| B8 | 0.003 | 17.89 |
| B9 | 0.001 | 14.33 |
| B10 | 0.006 | 16.55 |
| C1 | 0.029 | 13.59 |
| C2 | 0.029 | 11.95 |
| C3 | 0.077 | 17.50 |
| C4 | 0.017 | 19.82 |

Table 3 summarizes in vitro assay data related to BRD9 degradation in the presence of exemplary compounds.

| | BRD9 | BRD9 |
|---|---|---|
| Compound No | DC50 (μM) | Deg Amax % |
| D1 | 0.016 | 14.78 |
| D2 | 0.015 | 13.05 |
| D3 | 0.047 | 17.62 |
| D4 | 0.003 | 16.34 |
| E1 | 0.006 | 19.40 |
| E2 | 0.035 | 12.45 |
| E3 | 0.002 | 18.38 |
| E4 | 0.034 | 19.61 |
| E5 | 0.005 | 14.46 |
| E6 | 0.009 | 21.29 |
| E7 | 0.004 | 18.57 |
| E12 | 0.030 | 33.47 |
| E13 | 0.002 | 13.06 |
| E14 | 0.001 | 18.10 |
| E15 | 0.017 | 19.85 |
| E16 | 0.001 | 16.58 |
| E17 | 0.005 | 18.79 |
| E18 | 0.019 | 16.66 |
| E22 | 0.034 | 39.25 |
| E23 | 0.011 | 14.27 |
| E24 | 0.042 | 17.08 |
| E25 | 0.001 | 13.02 |
| E27 | 0.033 | 22.28 |
| E28 | 0.021 | 23.20 |
| E29 | 0.001 | 19.48 |
| E30 | 0.003 | 11.19 |
| E31 | 0.001 | 16.43 |
| E32 | 0.001 | 17.58 |
| E33 | 0.005 | 14.29 |
| E34 | 0.002 | 13.08 |
| E35 | 0.006 | 15.90 |
| E36 | 0.006 | 12.80 |
| E37 | 0.009 | 13.77 |
| E39 | 0.004 | 14.60 |
| E40 | 0.029 | 10.51 |
| E42 | 0.004 | 17.34 |
| E43 | 0.003 | 17.45 |
| E45 | 0.007 | 15.28 |
| E46 | 0.033 | 15.69 |
| E47 | 0.049 | 32.89 |
| E48 | 0.002 | 15.41 |
| E49 | 0.007 | 19.41 |
| E50 | 0.008 | 13.47 |
| E51 | 0.007 | 11.66 |
| E52 | 0.004 | 14.99 |
| E53 | 0.004 | 16.11 |
| E54 | 0.004 | 18.33 |
| E55 | 0.005 | 18.12 |
| E56 | 0.001 | 19.65 |

Having thus described several aspects of several embodiments, it is to be appreciated various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only.

Those skilled in the art will recognize, or be able to ascertain, using no more than routine experimentation, numerous equivalents to the specific embodiments described specifically herein. Such equivalents are intended to be encompassed in the scope of the following claims.

We claim:

1. A compound of Formula (A) or a pharmaceutically acceptable salt, hydrate, solvate, prodrug, stereoisomer, or tautomer thereof, wherein: 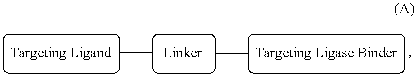
the Targeting Ligand is a group that is capable of binding to a bromodomain-containing protein, e.g., BRD9;
the Linker is a group that covalently links the Targeting Ligand to the Targeting Ligase Binder;
the Targeting Ligase Binder is a group that is capable of binding to a ligase (e.g., Cereblon E3 Ubiquitin ligase).

2. The compound of claim 1, wherein the Targeting Ligand is a compound of Formula (TL-I): 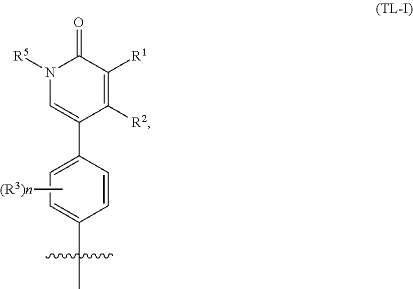
or a pharmaceutically acceptable salt, hydrate, solvate, prodrug, stereoisomer, or tautomer thereof, wherein:
R¹ and R² are independently selected from the group consisting of hydrogen and C₁₋₆ alkyl; or R¹ and R² together with the atoms to which they are attached form an aryl or heteroaryl;
R³ are each independently selected from the group consisting of C₁₋₆ alkyl, C₁₋₆ alkoxyl, and halogen;
R⁵ is selected from the group consisting of hydrogen and C₁₋₆ alkyl;
n is 0, 1, or 2.

3. The compound of any one of the preceding claims or a pharmaceutically acceptable salt, hydrate, solvate, prodrug, stereoisomer, or tautomer thereof, wherein the Targeting Ligand is a compound of Formula (TL-II):

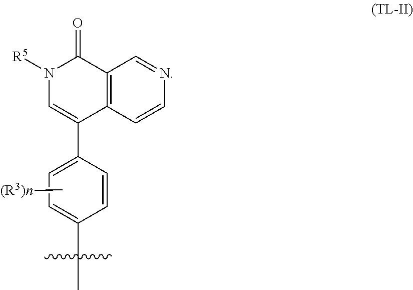

4. The compound of claim 1, wherein the Targeting Ligand is a compound of Formula (TL-I′): 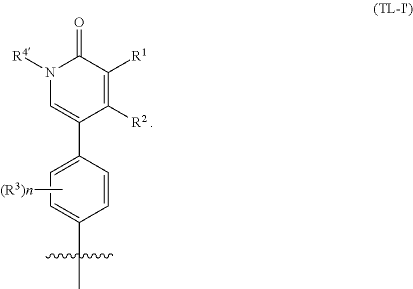
or a pharmaceutically acceptable salt, hydrate, solvate, prodrug, stereoisomer, or tautomer thereof, wherein:
R¹ and R² are independently selected from the group consisting of hydrogen and C₁₋₆ alkyl; or R¹ and R² together with the atoms to which they are attached form an aryl or heteroaryl;
R³ are each independently selected from the group consisting of C₁₋₆ alkyl, C₁₋₆ alkoxyl, and halogen;
R^{4′} is selected from the group consisting of hydrogen and C₁₋₆ alkyl; and
n is 0, 1, or 2.

5. The compound of claim 1 or 4, or a pharmaceutically acceptable salt, hydrate, solvate, prodrug, stereoisomer, or tautomer thereof, wherein the Targeting Ligand is a compound of Formula (TL-II′):

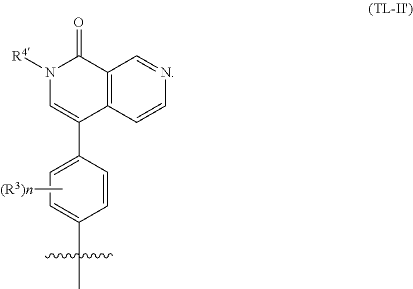

6. The compound of any one of the preceding claims or a pharmaceutically acceptable salt, hydrate, solvate, prodrug, stereoisomer, or tautomer thereof, wherein the Targeting Ligand is selected from the group consisting of:

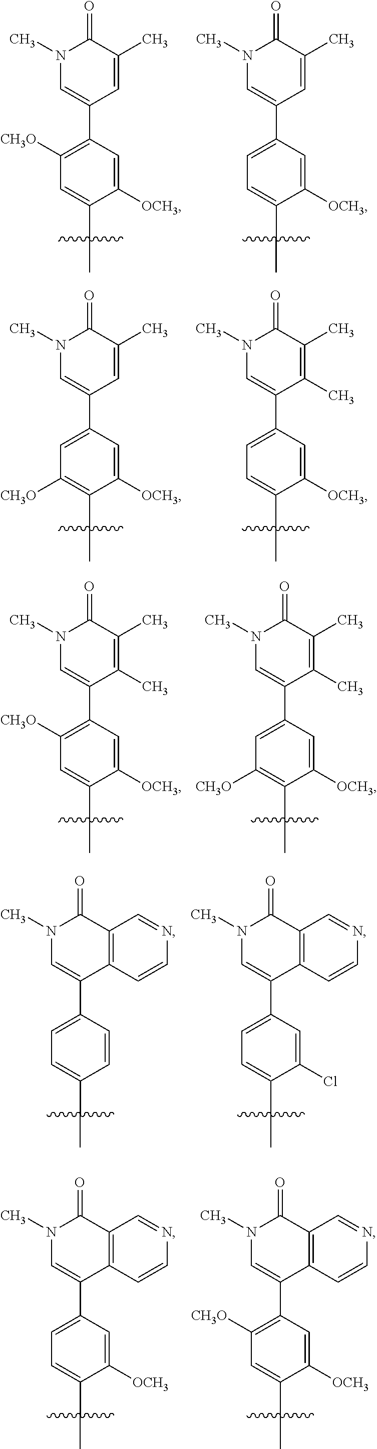

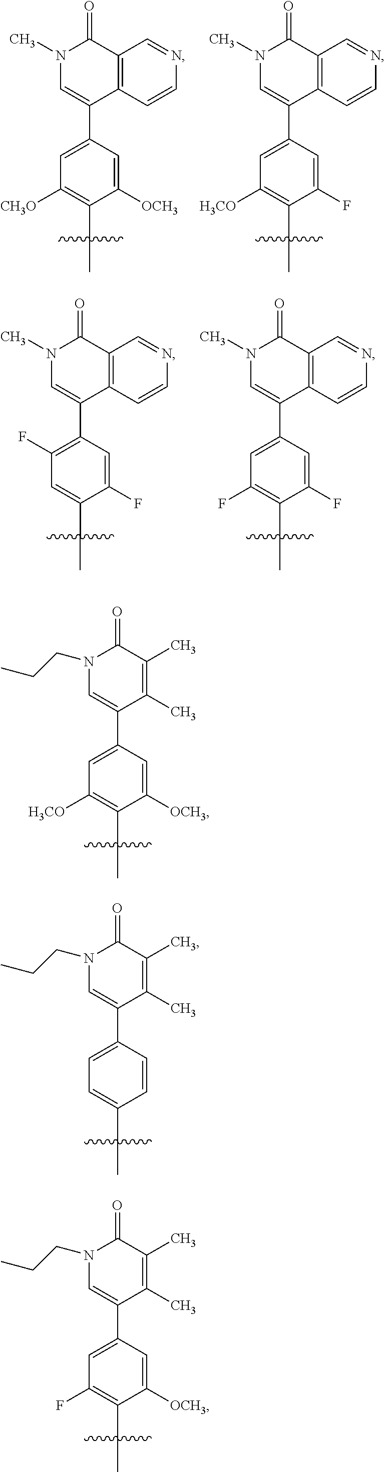

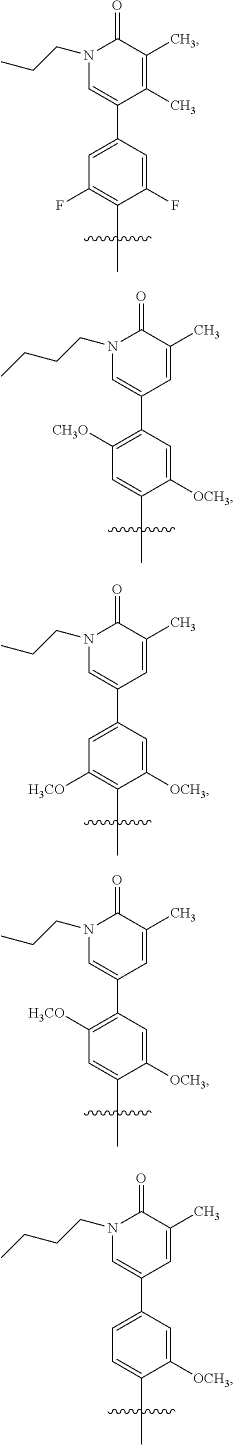

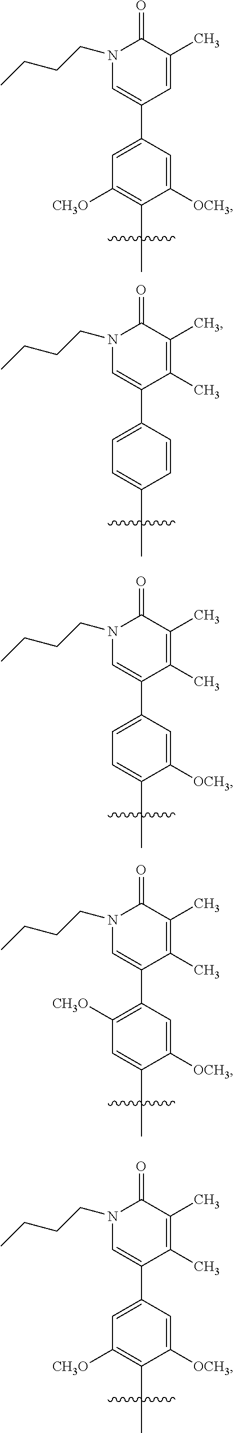

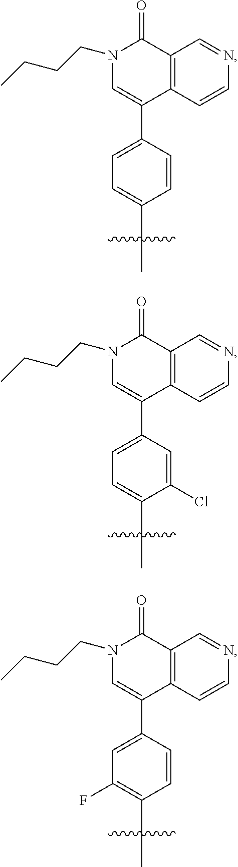 [SMILES: *c1ccc(-c2cn(CCCC)c(=O)c3cnccc23)cc1 ; *c1ccc(-c2cn(CCCC)c(=O)c3cnccc23)cc1Cl ; *c1ccc(-c2cn(CCCC)c(=O)c3cnccc23)cc1F]

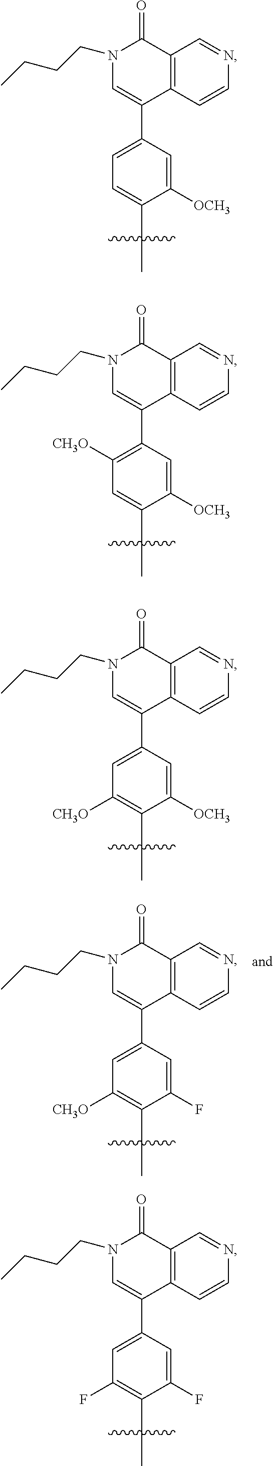

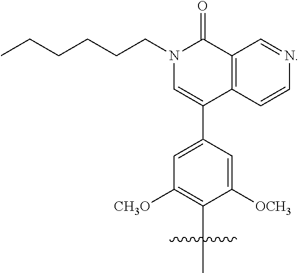

7. The compound of any one of the preceding claims, wherein the Targeting Ligase Binder is a compound of Formula (TLB-I): 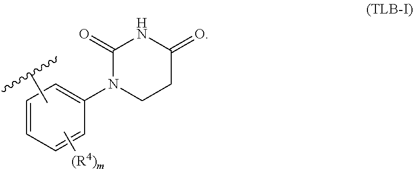
or a pharmaceutically acceptable salt, hydrate, solvate, prodrug, stereoisomer, or tautomer thereof, wherein:
R⁴ is selected from the group consisting of C₁₋₆ alkyl, C₁₋₆ alkoxyl, and halogen; and
m is 0, 1, or 2.

8. The compound of any one of claims 1-6, wherein the Targeting Ligase Binder is a compound of Formula (TLB-I′): 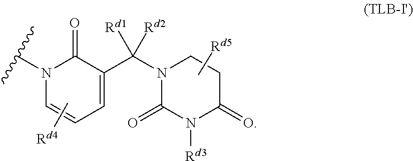
or a pharmaceutically acceptable salt, hydrate, solvate, prodrug, stereoisomer, or tautomer thereof, wherein R^{d1} and R^{d2} are each independently selected from the group consisting of H, C₁₋₆ alkyl, C₁₋₆ alkoxyl, C₁₋₆ haloalkyl, and C₁₋₆ heteroalkyl; R^{d3} is H; R^{d4} is selected from the group consisting of H, C₁₋₆ alkyl, halo, C₁₋₆ haloalkyl, and C₁₋₆ heteroalkyl; and R^{d5} is selected from the group consisting of H, C₁₋₆ alkyl, halo, C₁₋₆ haloalkyl, and C₁₋₆ heteroalkyl.

9. The compound of any one of the preceding claims, wherein the Linker is a compound of Formula (L-I): 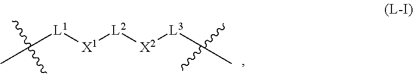
or a pharmaceutically acceptable salt, hydrate, solvate, prodrug, stereoisomer, or tautomer thereof, wherein:
L¹ is selected from the group consisting of a bond, O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the Targeting Ligand;
X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;
L² is selected from the group consisting of a bond, O, NR′, C₁₋₆ alkylene, and C₁₋₆ heteroalkylene; or
X¹-L²-X² form a spiroheterocyclyl; and
L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O , wherein * denotes the point of attachment of L³ to X² in Formula (L-I); wherein no more than 2 of L¹, X¹, X², L², and L³ can simultaneously be a bond.

10. The compound of any one of the preceding claims, wherein X¹-L²-X² is selected from the group consisting of: 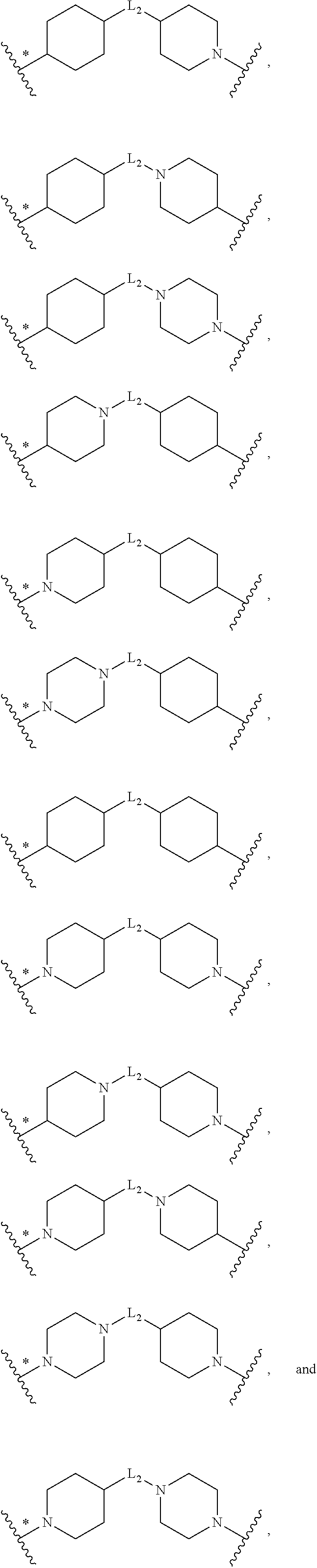
or a pharmaceutically acceptable salt, hydrate, solvate, prodrug, stereoisomer, or tautomer thereof, wherein * denotes the point of attachment to L¹.

11. The compound of any one of the preceding claims, wherein the Linker is selected from the group consisting of: 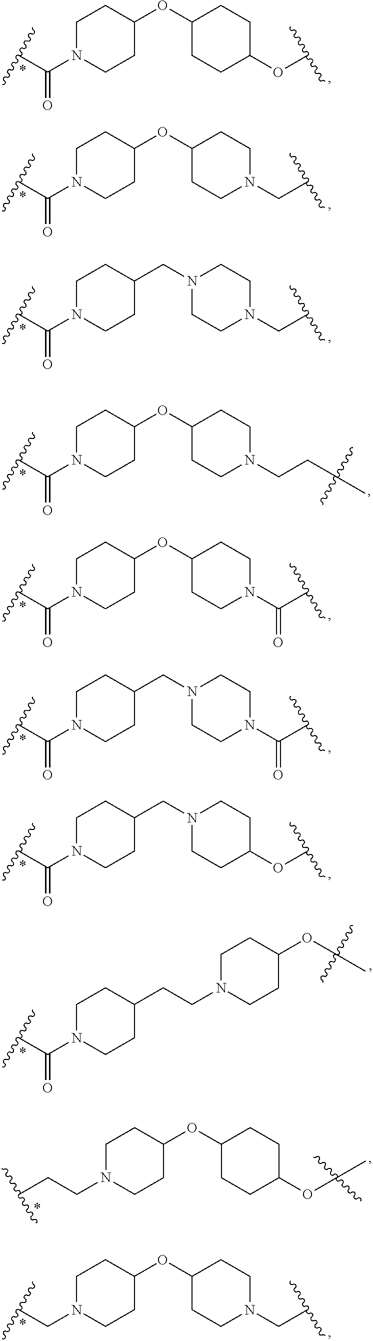 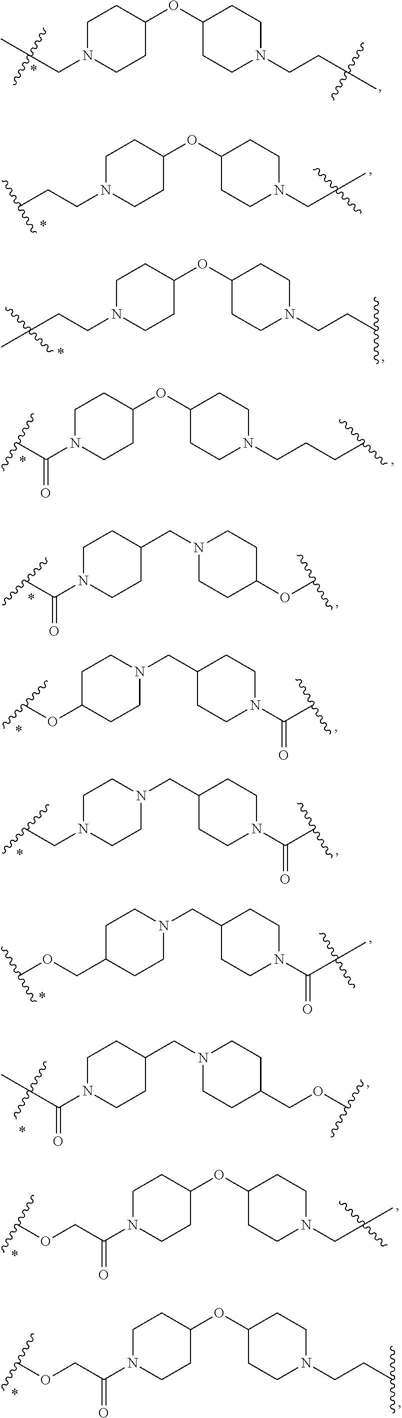 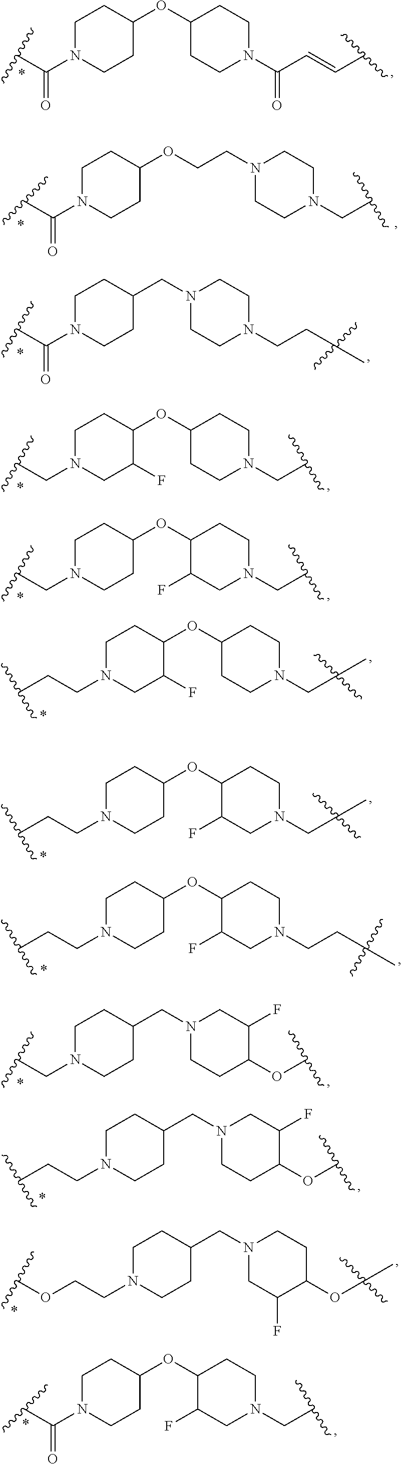 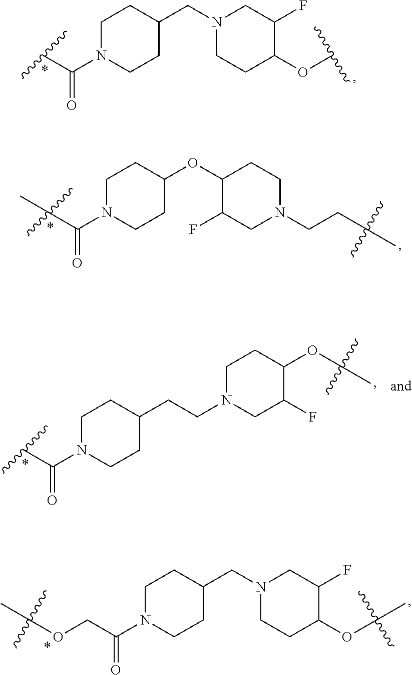 [SMILES: *OC1CCN(CC2CCN(C(*)=O)CC2)CC1F ; *CCN1CCC(OC2CCN(C(*)=O)CC2)C(F)C1 ; *OC1CCN(CCC2CCN(C(*)=O)CC2)CC1F ; *OCC(=O)N1CCC(CN2CCC(O*)C(F)C2)CC1]
or a pharmaceutically acceptable salt, hydrate, solvate, prodrug, stereoisomer, or tautomer thereof, wherein * denotes the point of attachment to the Targeting Ligase Binder.

12. The compound of any one of the preceding claims, wherein the compound is a compound of Formula (BF-I) or (BF-I′): 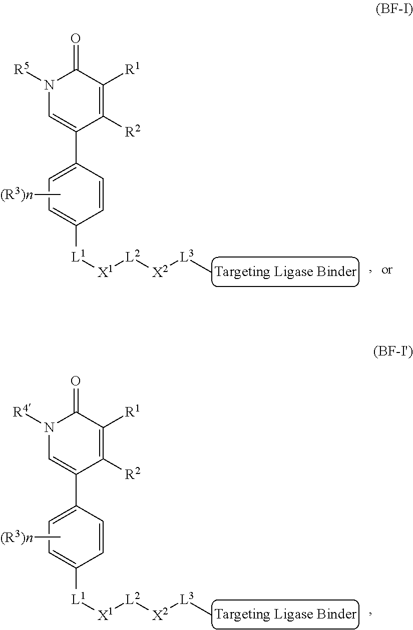
or a pharmaceutically acceptable salt, hydrate, solvate, prodrug, stereoisomer, or tautomer thereof, wherein:
R¹ and R² are independently selected from the group consisting of hydrogen and C₁₋₆ alkyl; or R¹ and R² together with the atoms to which they are attached form an aryl or heteroaryl;
R³ is selected from the group consisting of C₁₋₆ alkyl, C₁₋₆ alkoxyl, and halogen;
L¹ is selected from the group consisting of a bond, O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the phenyl ring in Formula (BF-I) or (BF-I′);
X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;
L² is selected from the group consisting of a bond, O, NR′, C₁₋₆ alkylene, and C₁₋₆ heteroalkylene; or
X¹-L²-X² form a spiroheterocyclyl;
L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O , wherein * denotes the point of attachment of L³ to X² in Formula (BF-I) or (BF-I′); wherein no more than 2 of L¹, X¹, X², L², and L³ can simultaneously be a bond;
R⁵ is selected from the group consisting of hydrogen and C₁₋₆ alkyl;
R^{4′} is selected from the group consisting of hydrogen or C₁₋₆ alkyl
R′ is selected from the group consisting of hydrogen and C₁₋₆ alkyl; and
n is 0, 1, or 2; and
the Targeting Ligase Binder is a group that is capable of binding to a Ubiquitin ligase, e.g., an E3 Ubiquitin ligase, such as Cereblon.

13. The compound of any one of claims 1-3, 6, 7, and 9-12, wherein the compound is a compound of Formula (BF-II): 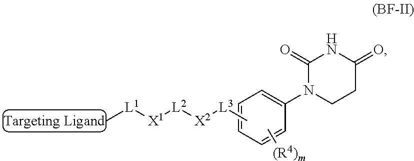
or a pharmaceutically acceptable salt, hydrate, solvate, prodrug, stereoisomer, or tautomer thereof, wherein:
L¹ is selected from the group consisting of a bond, O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the Targeting Ligand in Formula (BF-II);
X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;
L² is selected from the group consisting of a bond, O, NR′, C₁₋₆ alkylene, and C₁₋₆ heteroalkylene; or
X¹-L²-X² form a spiroheterocyclyl;
L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O , wherein * denotes the point of attachment of L³ to X² in Formula (BF-II); wherein no more than 2 of L¹, X¹, X², L², and L³ can simultaneously be a bond;
R⁴ is selected from the group consisting of C₁₋₆ alkyl, C₁₋₆ alkoxyl, and halogen;
R′ is selected from the group consisting of hydrogen and C₁₋₆ alkyl; and
m is 0, 1, or 2; and
the Targeting Ligand is a group that is capable of binding to a bromodomain-containing protein, e.g., BRD9.

14. The compound of any one of claims 1-3, 6, 7, and 9-13, wherein the compound is a compound of Formula (BF-III) or a pharmaceutically acceptable salt, hydrate, solvate, prodrug, stereoisomer, or tautomer thereof, wherein: 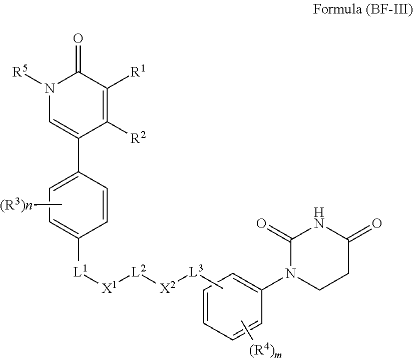
R¹ and R² are independently selected from the group consisting of hydrogen and C₁₋₆ alkyl; or R¹ and R² together with the atoms to which they are attached form an aryl or heteroaryl;
R³ is selected from the group consisting of C₁₋₆ alkyl, C₁₋₆ alkoxyl, and halogen;
L¹ is selected from the group consisting of a bond, O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the phenyl ring in Formula (BF-III);
X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;
L² is selected from the group consisting of a bond, O, NR′, C₁₋₆ alkylene, and C₁₋₆ heteroalkylene; or
X¹-L²-X² form a spiroheterocyclyl;
L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O , wherein * denotes the point of attachment of L³ to X² in Formula (BF-III); wherein no more than 2 of L¹, X¹, X², L², and L³ can simultaneously be a bond;
R⁴ is selected from the group consisting of OH, C₁₋₆ alkyl, C₁₋₆ alkoxyl, and halogen;
R⁵ is selected from the group consisting of hydrogen and C₁₋₆ alkyl;
R′ is selected from the group consisting of hydrogen and C₁₋₆ alkyl; and
m and n are each independently 0, 1, or 2.

15. The compound of any one of claims 1-3, 6, 7, and 9-14, wherein the compound is a compound of Formula (BF-III) or a pharmaceutically acceptable salt, hydrate, solvate, prodrug, stereoisomer, or tautomer thereof,
wherein:
R¹ and R² are independently selected from the group consisting of hydrogen and C₁₋₆ alkyl; or R¹ and R² together with the atoms to which they are attached form a heteroaryl;
R³ is selected from the group consisting of C₁₋₆ alkoxyl and halogen;
L¹ is selected from the group consisting of O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L to the phenyl ring in Formula (BF-III);
X¹-L²-X² is selected from the group consisting of: 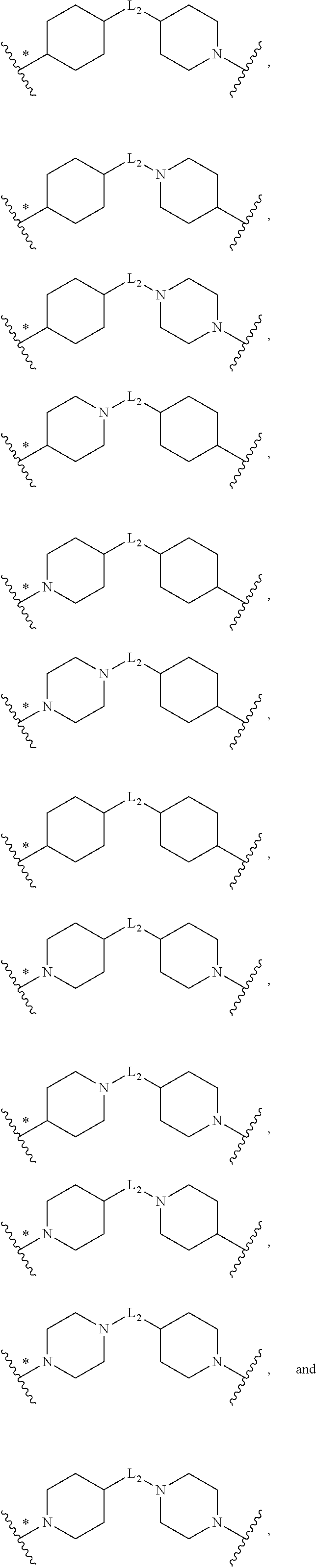
wherein * denotes the point of attachment to L¹, and wherein the carbocyclyl and heterocyclyl are substituted with 0-4 occurrences of R^{a}, wherein each R^{a} is halogen;
L² is selected from the group consisting of O, C₁₋₆ alkylene, and C₁₋₆ heteroalkylene;
L³ is selected from the group consisting of O, C(O), C₁₋₆ alkylene, C₁₋₆ heteroalkylene, and *C(O)—C₁₋₆ alkylene-O, wherein * denotes the point of attachment of L³ to X² in Formula (BF-III); wherein no more than 2 of L¹, X¹, X², L², and L³ can simultaneously be a bond;
R⁴ is selected from the group consisting of OH, C₁₋₆ alkyl, C₁₋₆ alkoxyl, and halogen;
R⁵ is C₁₋₆ alkyl; and
m and n are each independently 0, 1, or 2.

16. The compound of any one of claims 1-3, 6, 7, and 9-15, or a pharmaceutically acceptable salt, hydrate, solvate, prodrug, stereoisomer, or tautomer thereof, selected from the group consisting of:

| Compound | |
|---|---|
| Number | Compound Structure |
| A1 | 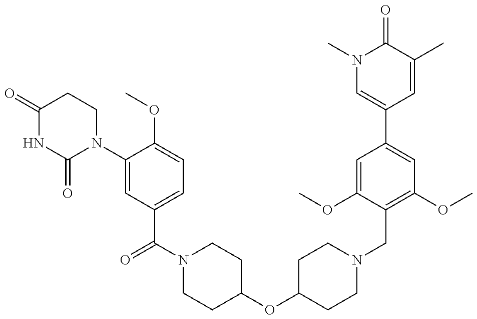 [SMILES: COc1ccc(C(=O)N2CCC(OC3CCN(Cc4c(OC)cc(-c5cc(C)c(=O)n(C)c5)cc4OC)CC3)CC2)cc1N1CCC(=O)NC1=O] |
| A2 | 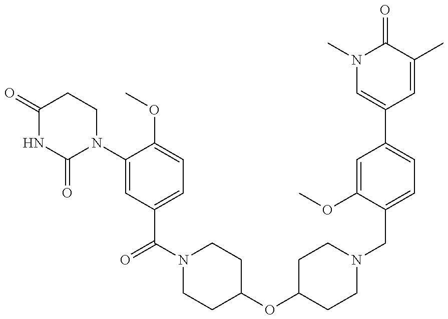 [SMILES: COc1cc(-c2cc(C)c(=O)n(C)c2)ccc1CN1CCC(OC2CCN(C(=O)c3ccc(OC)c(N4CCC(=O)NC4=O)c3)CC2)CC1] |
| A3 | 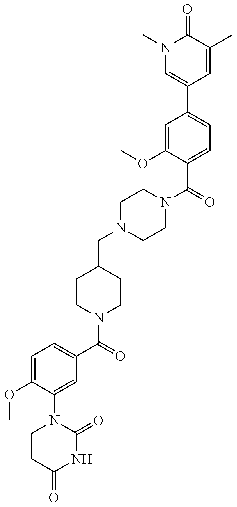 [SMILES: COc1cc(-c2cc(C)c(=O)n(C)c2)ccc1C(=O)N1CCN(CC2CCN(C(=O)c3ccc(OC)c(N4CCC(=O)NC4=O)c3)CC2)CC1] |
| A4 | 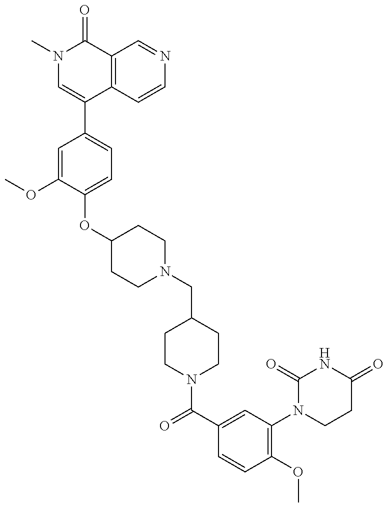 [SMILES: COc1cc(-c2cn(C)c(=O)c3cnccc23)ccc1OC1CCN(CC2CCN(C(=O)c3ccc(OC)c(N4CCC(=O)NC4=O)c3)CC2)CC1] |
| A5 | 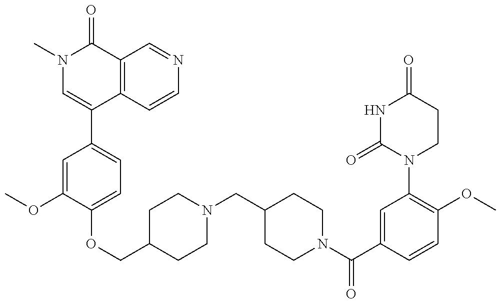 |
| A6 | 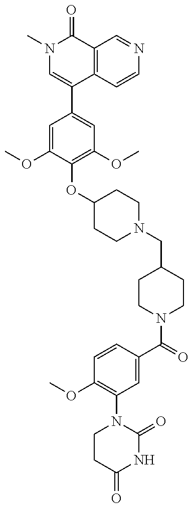 [SMILES: COc1ccc(C(=O)N2CCC(CN3CCC(Oc4c(OC)cc(-c5cn(C)c(=O)c6cnccc56)cc4OC)CC3)CC2)cc1N1CCC(=O)NC1=O] |
| A7 | 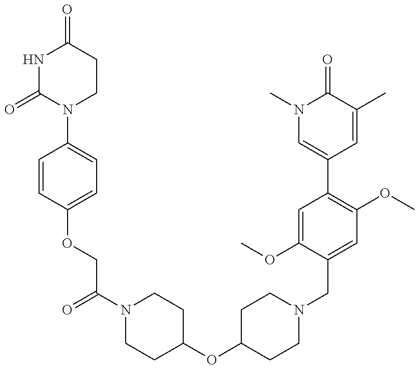 [SMILES: COc1cc(-c2cc(C)c(=O)n(C)c2)c(OC)cc1CN1CCC(OC2CCN(C(=O)COc3ccc(N4CCC(=O)NC4=O)cc3)CC2)CC1] |
| A8 | 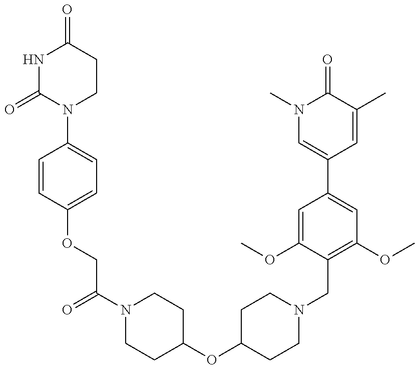 [SMILES: COc1cc(-c2cc(C)c(=O)n(C)c2)cc(OC)c1CN1CCC(OC2CCN(C(=O)COc3ccc(N4CCC(=O)NC4=O)cc3)CC2)CC1] |
| A9 | 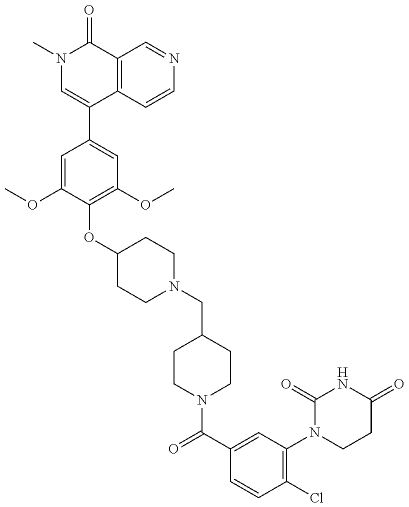 [SMILES: COc1cc(-c2cn(C)c(=O)c3cnccc23)cc(OC)c1OC1CCN(CC2CCN(C(=O)c3ccc(Cl)c(N4CCC(=O)NC4=O)c3)CC2)CC1] |
| A10 | 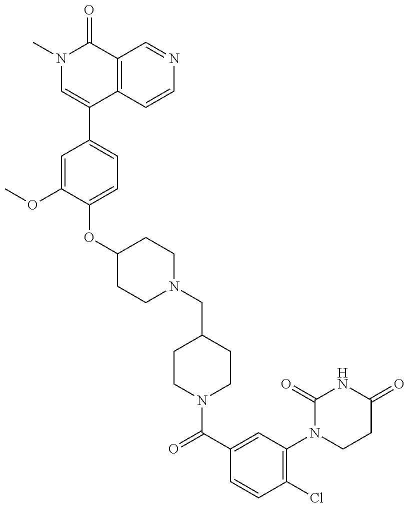 [SMILES: COc1cc(-c2cn(C)c(=O)c3cnccc23)ccc1OC1CCN(CC2CCN(C(=O)c3ccc(Cl)c(N4CCC(=O)NC4=O)c3)CC2)CC1] |
| A11 | 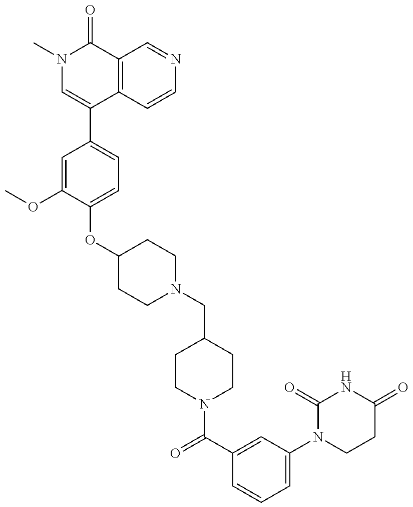 [SMILES: COc1cc(-c2cn(C)c(=O)c3cnccc23)ccc1OC1CCN(CC2CCN(C(=O)c3cccc(N4CCC(=O)NC4=O)c3)CC2)CC1] |
| A12 | 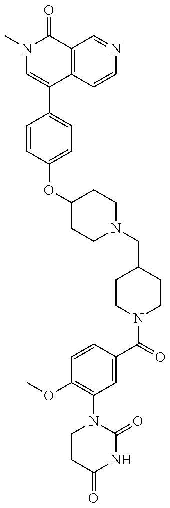 [SMILES: COc1ccc(C(=O)N2CCC(CN3CCC(Oc4ccc(-c5cn(C)c(=O)c6cnccc56)cc4)CC3)CC2)cc1N1CCC(=O)NC1=O] |
| A13 | 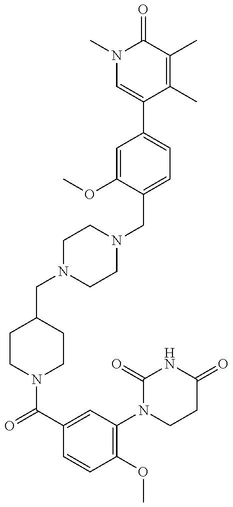 [SMILES: COc1cc(-c2cn(C)c(=O)c(C)c2C)ccc1CN1CCN(CC2CCN(C(=O)c3ccc(OC)c(N4CCC(=O)NC4=O)c3)CC2)CC1] |
| A14 | 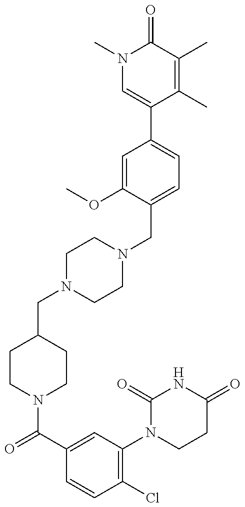 [SMILES: COc1cc(-c2cn(C)c(=O)c(C)c2C)ccc1CN1CCN(CC2CCN(C(=O)c3ccc(Cl)c(N4CCC(=O)NC4=O)c3)CC2)CC1] |
| A15 | 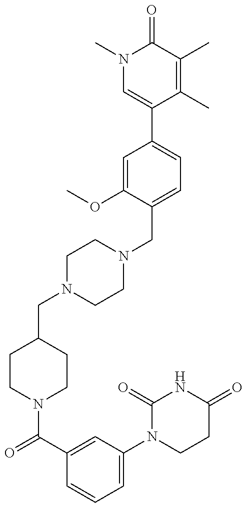 [SMILES: COc1cc(-c2cn(C)c(=O)c(C)c2C)ccc1CN1CCN(CC2CCN(C(=O)c3cccc(N4CCC(=O)NC4=O)c3)CC2)CC1] |
| A16 | 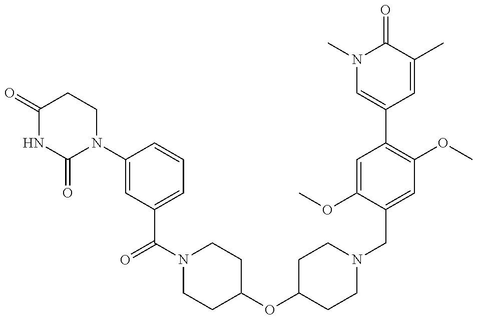 [SMILES: COc1cc(-c2cc(C)c(=O)n(C)c2)c(OC)cc1CN1CCC(OC2CCN(C(=O)c3cccc(N4CCC(=O)NC4=O)c3)CC2)CC1] |
| A17 | 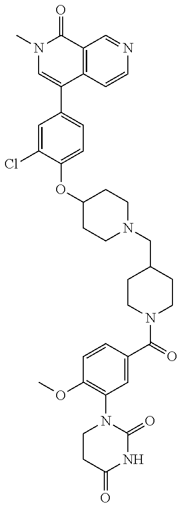 [SMILES: COc1ccc(C(=O)N2CCC(CN3CCC(Oc4ccc(-c5cn(C)c(=O)c6cnccc56)cc4Cl)CC3)CC2)cc1N1CCC(=O)NC1=O] |
| A18 | 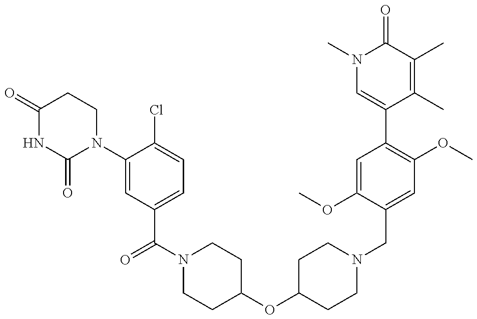 [SMILES: COc1cc(-c2cn(C)c(=O)c(C)c2C)c(OC)cc1CN1CCC(OC2CCN(C(=O)c3ccc(Cl)c(N4CCC(=O)NC4=O)c3)CC2)CC1] |
| A19 | 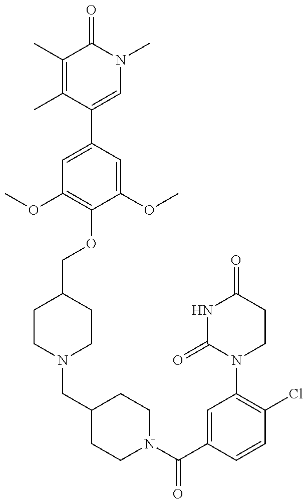 [SMILES: COc1cc(-c2cn(C)c(=O)c(C)c2C)cc(OC)c1OCC1CCN(CC2CCN(C(=O)c3ccc(Cl)c(N4CCC(=O)NC4=O)c3)CC2)CC1] |
| A20 | 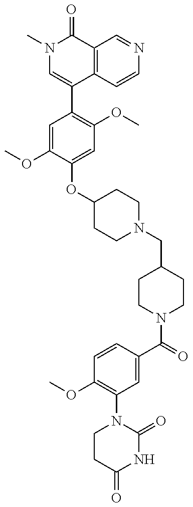 [SMILES: COc1cc(-c2cn(C)c(=O)c3cnccc23)c(OC)cc1OC1CCN(CC2CCN(C(=O)c3ccc(OC)c(N4CCC(=O)NC4=O)c3)CC2)CC1] |
| A21 | 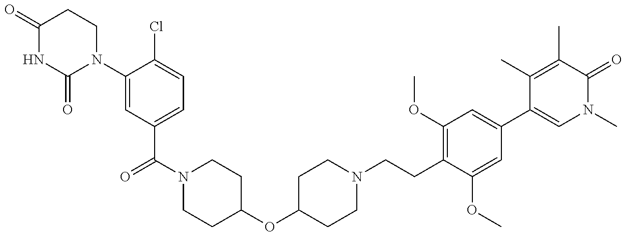 [SMILES: COc1cc(-c2cn(C)c(=O)c(C)c2C)cc(OC)c1CCN1CCC(OC2CCN(C(=O)c3ccc(Cl)c(N4CCC(=O)NC4=O)c3)CC2)CC1] |
| A22 | 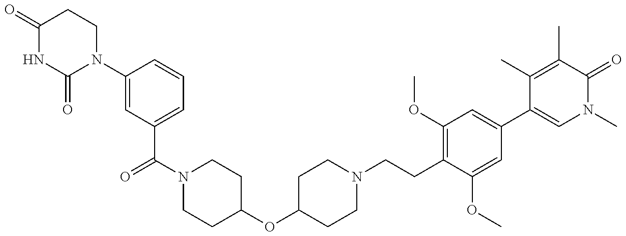 [SMILES: COc1cc(-c2cn(C)c(=O)c(C)c2C)cc(OC)c1CCN1CCC(OC2CCN(C(=O)c3cccc(N4CCC(=O)NC4=O)c3)CC2)CC1] |
| A23 | 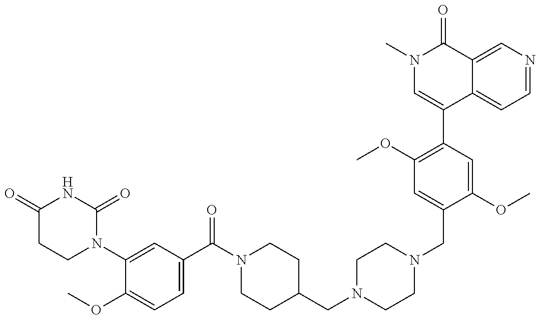 [SMILES: COc1cc(-c2cn(C)c(=O)c3cnccc23)c(OC)cc1CN1CCN(CC2CCN(C(=O)c3ccc(OC)c(N4CCC(=O)NC4=O)c3)CC2)CC1] |
| A24 | 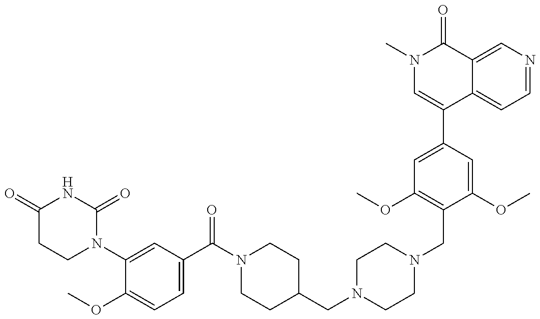 [SMILES: COc1ccc(C(=O)N2CCC(CN3CCN(Cc4c(OC)cc(-c5cn(C)c(=O)c6cnccc56)cc4OC)CC3)CC2)cc1N1CCC(=O)NC1=O] |
| A25 | 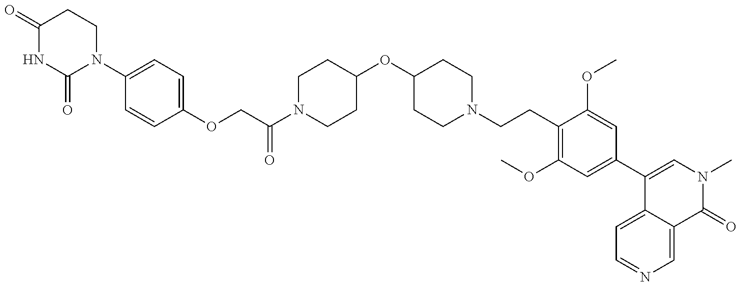 [SMILES: COc1cc(-c2cn(C)c(=O)c3cnccc23)cc(OC)c1CCN1CCC(OC2CCN(C(=O)COc3ccc(N4CCC(=O)NC4=O)cc3)CC2)CC1] |
| A26 | 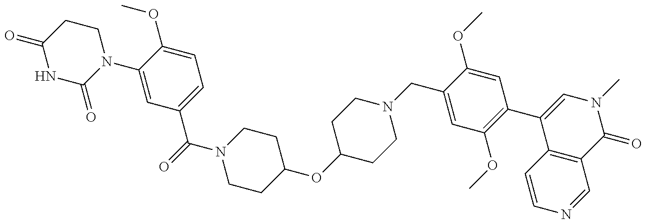 [SMILES: COc1cc(-c2cn(C)c(=O)c3cnccc23)c(OC)cc1CN1CCC(OC2CCN(C(=O)c3ccc(OC)c(N4CCC(=O)NC4=O)c3)CC2)CC1] |
| A27 | 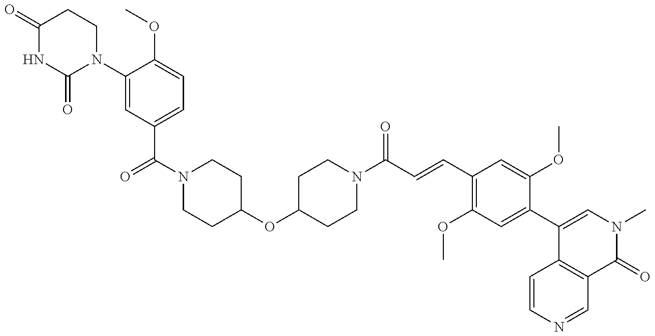 [SMILES: COc1cc(-c2cn(C)c(=O)c3cnccc23)c(OC)cc1/C=C/C(=O)N1CCC(OC2CCN(C(=O)c3ccc(OC)c(N4CCC(=O)NC4=O)c3)CC2)CC1] |
| A28 | 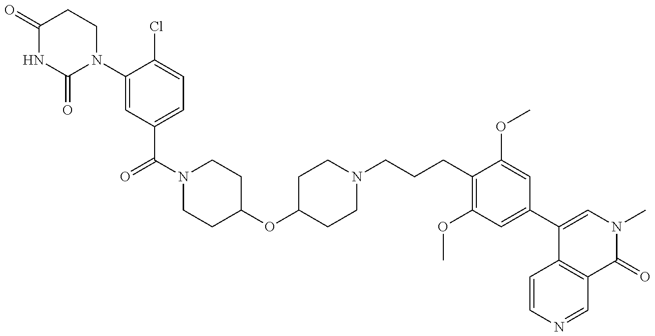 [SMILES: COc1cc(-c2cn(C)c(=O)c3cnccc23)cc(OC)c1CCCN1CCC(OC2CCN(C(=O)c3ccc(Cl)c(N4CCC(=O)NC4=O)c3)CC2)CC1] |
| A29 | 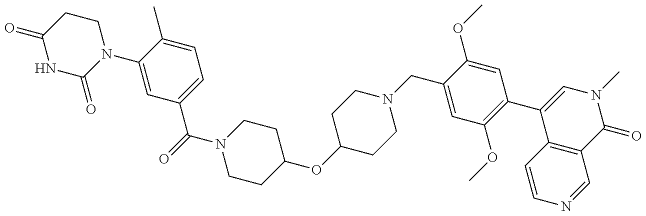 [SMILES: COc1cc(-c2cn(C)c(=O)c3cnccc23)c(OC)cc1CN1CCC(OC2CCN(C(=O)c3ccc(C)c(N4CCC(=O)NC4=O)c3)CC2)CC1] |
| A30 | 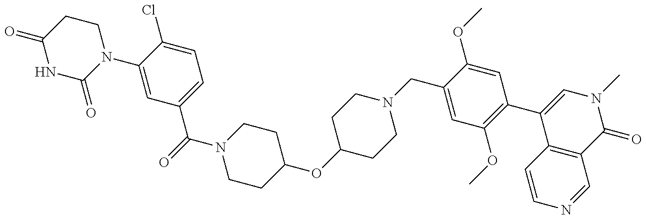 [SMILES: COc1cc(-c2cn(C)c(=O)c3cnccc23)c(OC)cc1CN1CCC(OC2CCN(C(=O)c3ccc(Cl)c(N4CCC(=O)NC4=O)c3)CC2)CC1] |
| A31 | 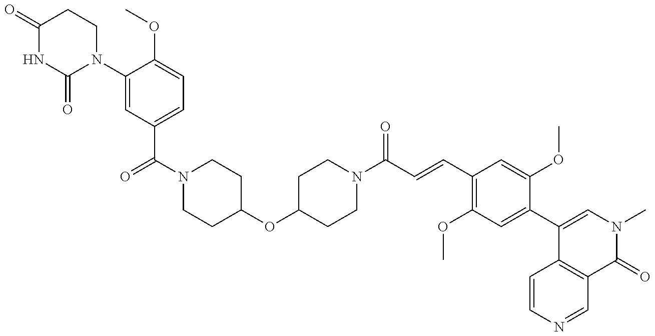 [SMILES: COc1cc(-c2cn(C)c(=O)c3cnccc23)c(OC)cc1/C=C/C(=O)N1CCC(OC2CCN(C(=O)c3ccc(OC)c(N4CCC(=O)NC4=O)c3)CC2)CC1] |
| A32 | 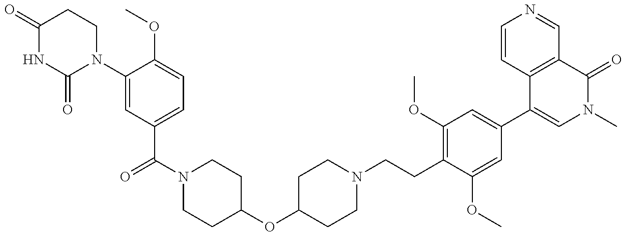 [SMILES: COc1ccc(C(=O)N2CCC(OC3CCN(CCc4c(OC)cc(-c5cn(C)c(=O)c6cnccc56)cc4OC)CC3)CC2)cc1N1CCC(=O)NC1=O] |
| A33 | 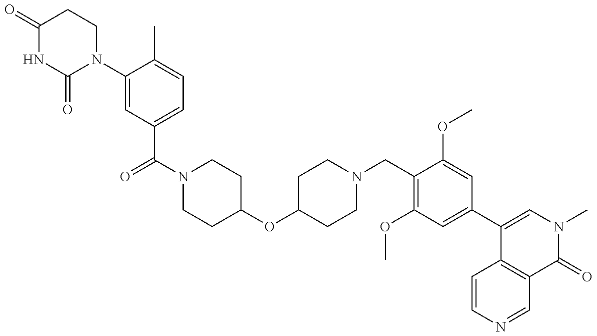 [SMILES: COc1cc(-c2cn(C)c(=O)c3cnccc23)cc(OC)c1CN1CCC(OC2CCN(C(=O)c3ccc(C)c(N4CCC(=O)NC4=O)c3)CC2)CC1] |
| A34 | 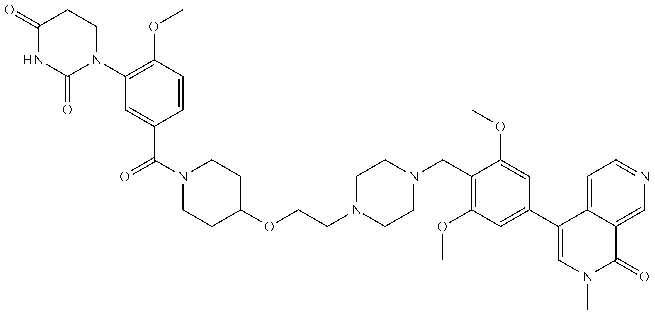 [SMILES: COc1ccc(C(=O)N2CCC(OCCN3CCN(Cc4c(OC)cc(-c5cn(C)c(=O)c6cnccc56)cc4OC)CC3)CC2)cc1N1CCC(=O)NC1=O] |
| A35 | 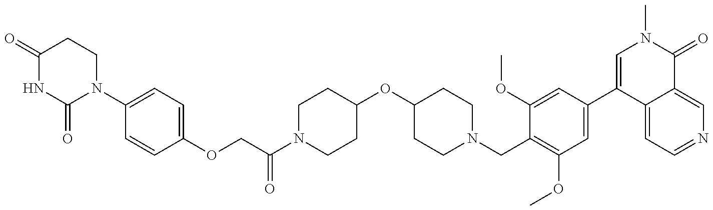 [SMILES: COc1cc(-c2cn(C)c(=O)c3cnccc23)cc(OC)c1CN1CCC(OC2CCN(C(=O)COc3ccc(N4CCC(=O)NC4=O)cc3)CC2)CC1] |
| A36 | 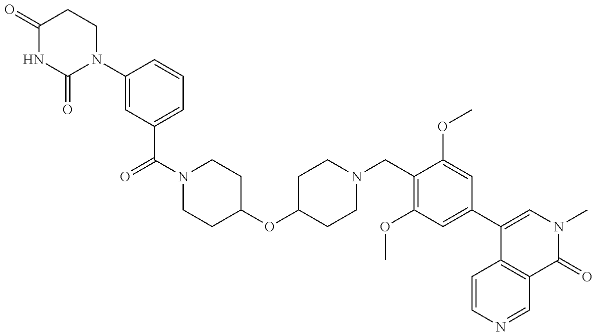 [SMILES: COc1cc(-c2cn(C)c(=O)c3cnccc23)cc(OC)c1CN1CCC(OC2CCN(C(=O)c3cccc(N4CCC(=O)NC4=O)c3)CC2)CC1] |
| A37 | 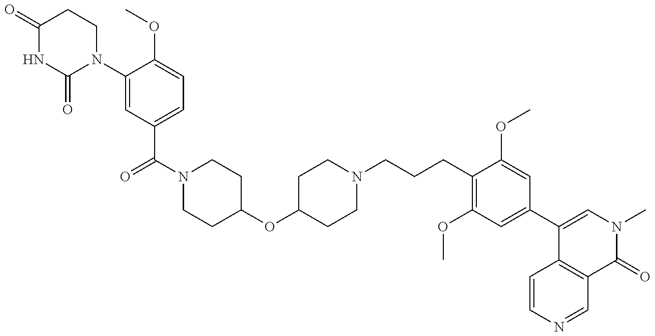 [SMILES: COc1ccc(C(=O)N2CCC(OC3CCN(CCCc4c(OC)cc(-c5cn(C)c(=O)c6cnccc56)cc4OC)CC3)CC2)cc1N1CCC(=O)NC1=O] |
| A38 | 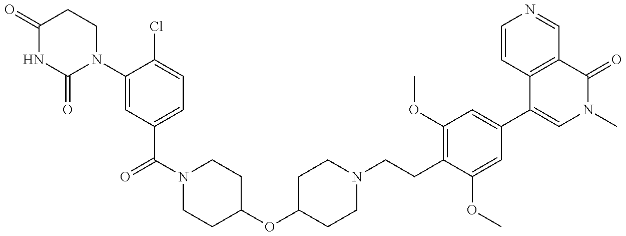 [SMILES: COc1cc(-c2cn(C)c(=O)c3cnccc23)cc(OC)c1CCN1CCC(OC2CCN(C(=O)c3ccc(Cl)c(N4CCC(=O)NC4=O)c3)CC2)CC1] |
| A39 | 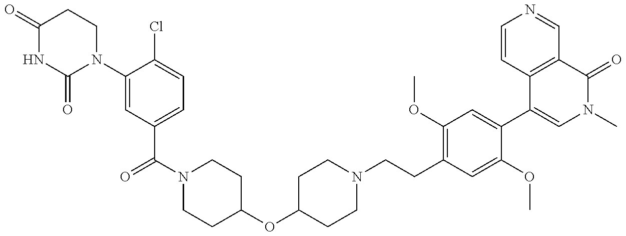 [SMILES: COc1cc(-c2cn(C)c(=O)c3cnccc23)c(OC)cc1CCN1CCC(OC2CCN(C(=O)c3ccc(Cl)c(N4CCC(=O)NC4=O)c3)CC2)CC1] |
| A40 | 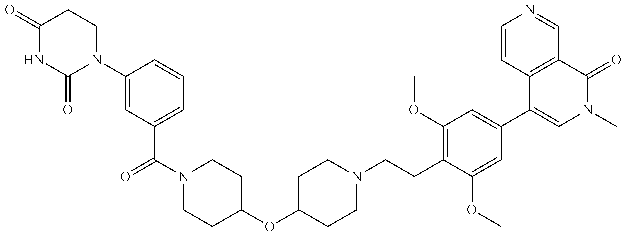 [SMILES: COc1cc(-c2cn(C)c(=O)c3cnccc23)cc(OC)c1CCN1CCC(OC2CCN(C(=O)c3cccc(N4CCC(=O)NC4=O)c3)CC2)CC1] |
| A41 | 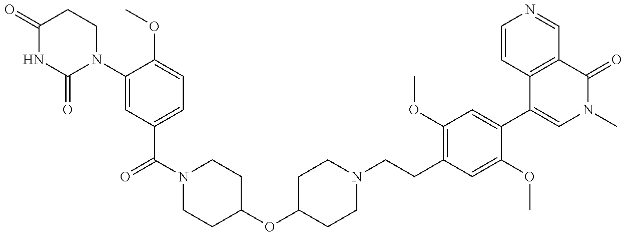 [SMILES: COc1cc(-c2cn(C)c(=O)c3cnccc23)c(OC)cc1CCN1CCC(OC2CCN(C(=O)c3ccc(OC)c(N4CCC(=O)NC4=O)c3)CC2)CC1] |
| A42 | 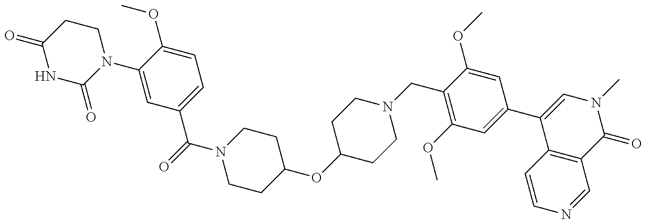 [SMILES: COc1ccc(C(=O)N2CCC(OC3CCN(Cc4c(OC)cc(-c5cn(C)c(=O)c6cnccc56)cc4OC)CC3)CC2)cc1N1CCC(=O)NC1=O] |
| B1 | 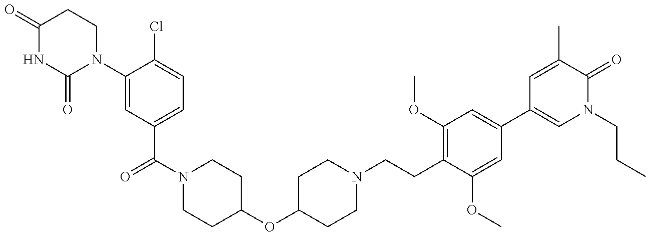 [SMILES: CCCn1cc(-c2cc(OC)c(CCN3CCC(OC4CCN(C(=O)c5ccc(Cl)c(N6CCC(=O)NC6=O)c5)CC4)CC3)c(OC)c2)cc(C)c1=O] |
| B2 | 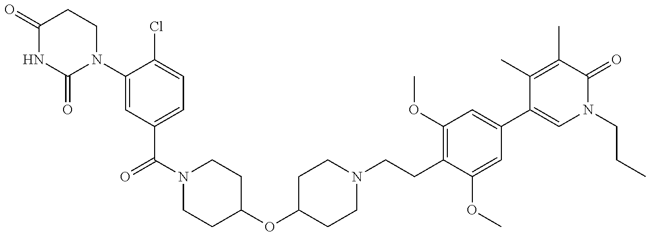 [SMILES: CCCn1cc(-c2cc(OC)c(CCN3CCC(OC4CCN(C(=O)c5ccc(Cl)c(N6CCC(=O)NC6=O)c5)CC4)CC3)c(OC)c2)c(C)c(C)c1=O] |
| B3 | 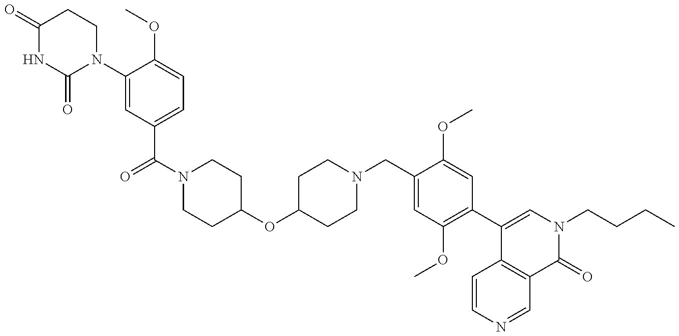 [SMILES: CCCCn1cc(-c2cc(OC)c(CN3CCC(OC4CCN(C(=O)c5ccc(OC)c(N6CCC(=O)NC6=O)c5)CC4)CC3)cc2OC)c2ccncc2c1=O] |
| B4 | 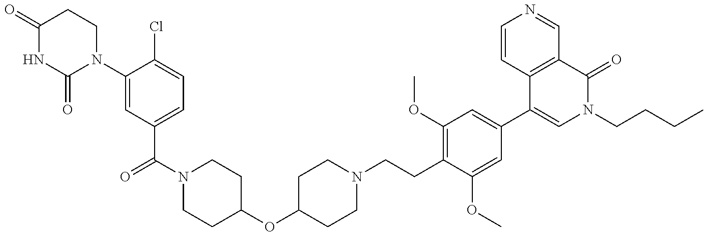 [SMILES: CCCCn1cc(-c2cc(OC)c(CCN3CCC(OC4CCN(C(=O)c5ccc(Cl)c(N6CCC(=O)NC6=O)c5)CC4)CC3)c(OC)c2)c2ccncc2c1=O] |
| B5 | 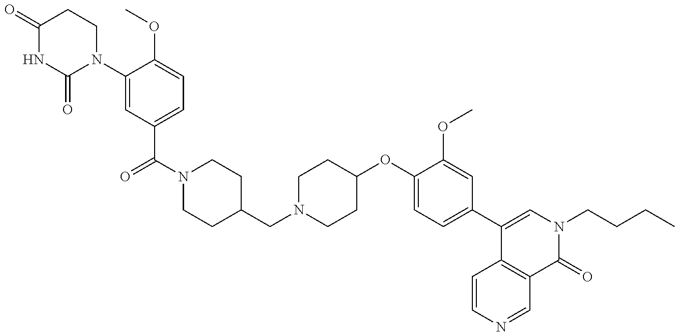 [SMILES: CCCCn1cc(-c2ccc(OC3CCN(CC4CCN(C(=O)c5ccc(OC)c(N6CCC(=O)NC6=O)c5)CC4)CC3)c(OC)c2)c2ccncc2c1=O] |
| B6 | 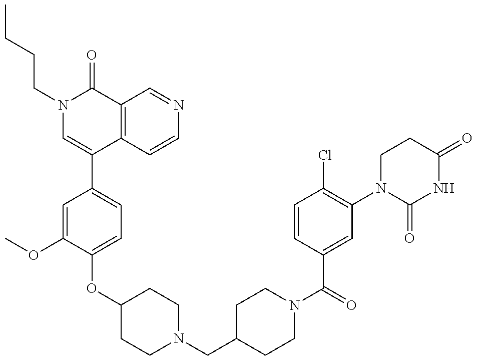 [SMILES: CCCCn1cc(-c2ccc(OC3CCN(CC4CCN(C(=O)c5ccc(Cl)c(N6CCC(=O)NC6=O)c5)CC4)CC3)c(OC)c2)c2ccncc2c1=O] |
| B7 | 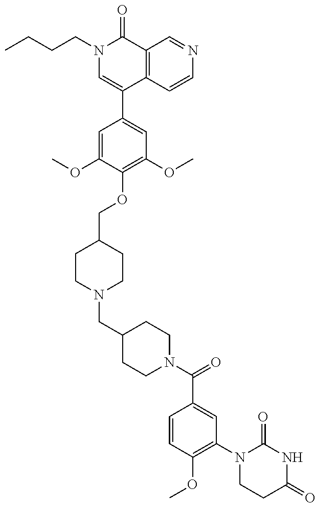 [SMILES: CCCCn1cc(-c2cc(OC)c(OCC3CCN(CC4CCN(C(=O)c5ccc(OC)c(N6CCC(=O)NC6=O)c5)CC4)CC3)c(OC)c2)c2ccncc2c1=O] |
| B8 | 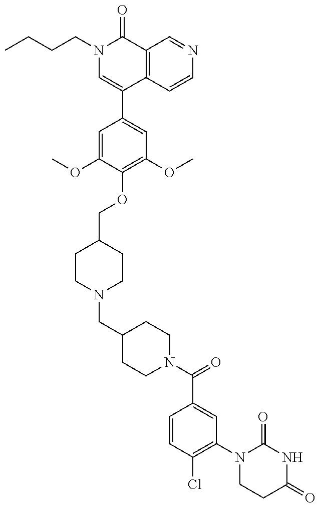 [SMILES: CCCCn1cc(-c2cc(OC)c(OCC3CCN(CC4CCN(C(=O)c5ccc(Cl)c(N6CCC(=O)NC6=O)c5)CC4)CC3)c(OC)c2)c2ccncc2c1=O] |
| B9 | 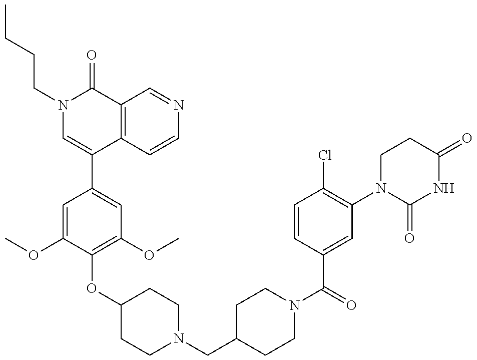 [SMILES: CCCCn1cc(-c2cc(OC)c(OC3CCN(CC4CCN(C(=O)c5ccc(Cl)c(N6CCC(=O)NC6=O)c5)CC4)CC3)c(OC)c2)c2ccncc2c1=O] |
| B10 | 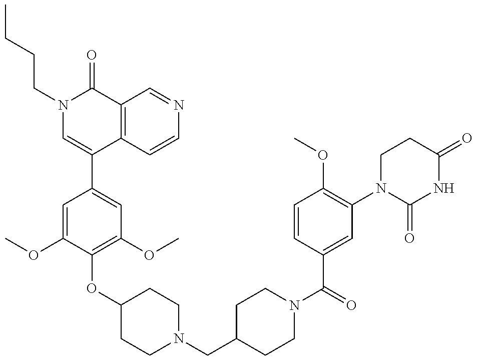 [SMILES: CCCCn1cc(-c2cc(OC)c(OC3CCN(CC4CCN(C(=O)c5ccc(OC)c(N6CCC(=O)NC6=O)c5)CC4)CC3)c(OC)c2)c2ccncc2c1=O] |
| E3 | 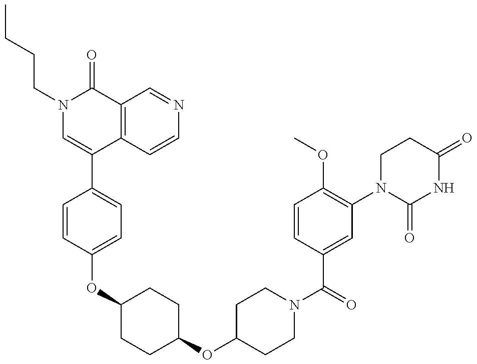 [SMILES: CCCCn1cc(-c2ccc(O[C@H]3CC[C@@H](OC4CCN(C(=O)c5ccc(OC)c(N6CCC(=O)NC6=O)c5)CC4)CC3)cc2)c2ccncc2c1=O] |
| E4 | 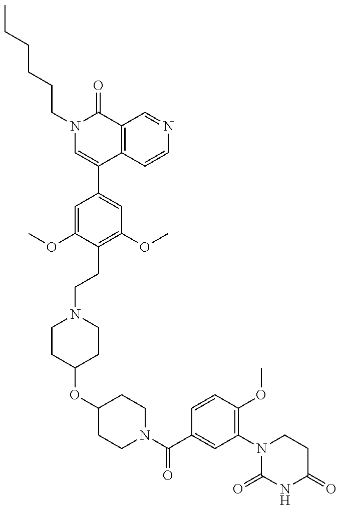 [SMILES: CCCCCCn1cc(-c2cc(OC)c(CCN3CCC(OC4CCN(C(=O)c5ccc(OC)c(N6CCC(=O)NC6=O)c5)CC4)CC3)c(OC)c2)c2ccncc2c1=O] |
| E5 | 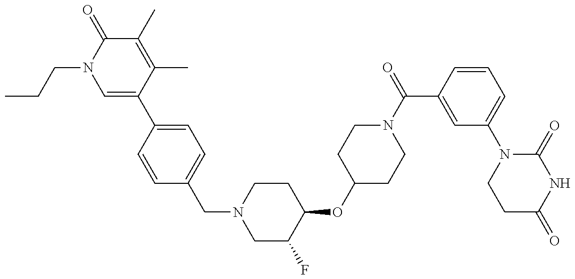 [SMILES: CCCn1cc(-c2ccc(CN3CC[C@@H](OC4CCN(C(=O)c5cccc(N6CCC(=O)NC6=O)c5)CC4)[C@H](F)C3)cc2)c(C)c(C)c1=O] |
| E6 | 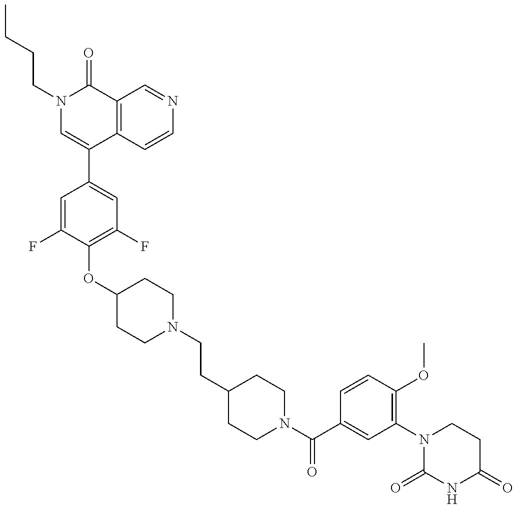 [SMILES: CCCCn1cc(-c2cc(F)c(OC3CCN(CCC4CCN(C(=O)c5ccc(OC)c(N6CCC(=O)NC6=O)c5)CC4)CC3)c(F)c2)c2ccncc2c1=O] |
| E12 | 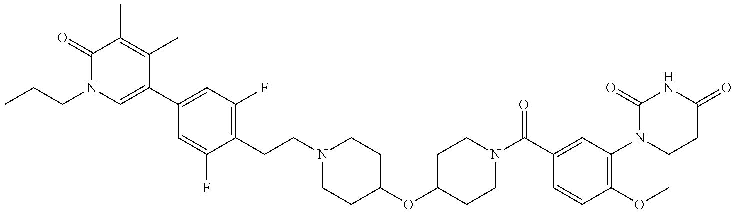 [SMILES: CCCn1cc(-c2cc(F)c(CCN3CCC(OC4CCN(C(=O)c5ccc(OC)c(N6CCC(=O)NC6=O)c5)CC4)CC3)c(F)c2)c(C)c(C)c1=O] |
| E13 | 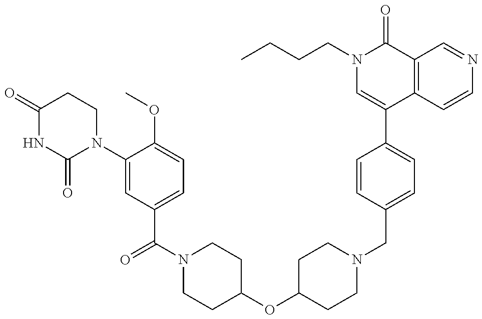 [SMILES: CCCCn1cc(-c2ccc(CN3CCC(OC4CCN(C(=O)c5ccc(OC)c(N6CCC(=O)NC6=O)c5)CC4)CC3)cc2)c2ccncc2c1=O] |
| E14 | 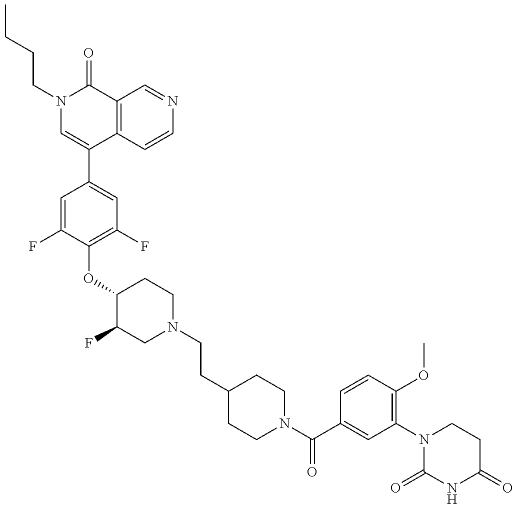 [SMILES: CCCCn1cc(-c2cc(F)c(O[C@@H]3CCN(CCC4CCN(C(=O)c5ccc(OC)c(N6CCC(=O)NC6=O)c5)CC4)C[C@H]3F)c(F)c2)c2ccncc2c1=O] |
| E15 | 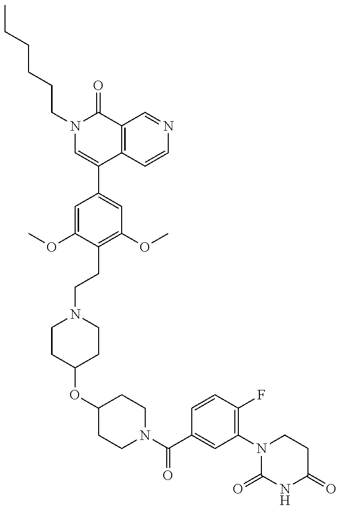 [SMILES: CCCCCCn1cc(-c2cc(OC)c(CCN3CCC(OC4CCN(C(=O)c5ccc(F)c(N6CCC(=O)NC6=O)c5)CC4)CC3)c(OC)c2)c2ccncc2c1=O] |
| E16 | 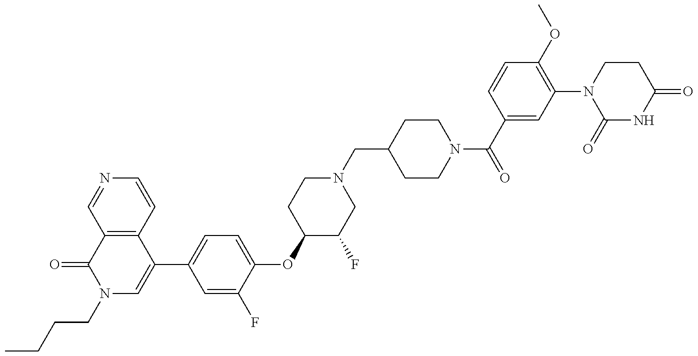 [SMILES: CCCCn1cc(-c2ccc(O[C@H]3CCN(CC4CCN(C(=O)c5ccc(OC)c(N6CCC(=O)NC6=O)c5)CC4)C[C@@H]3F)c(F)c2)c2ccncc2c1=O] |
| E22 | 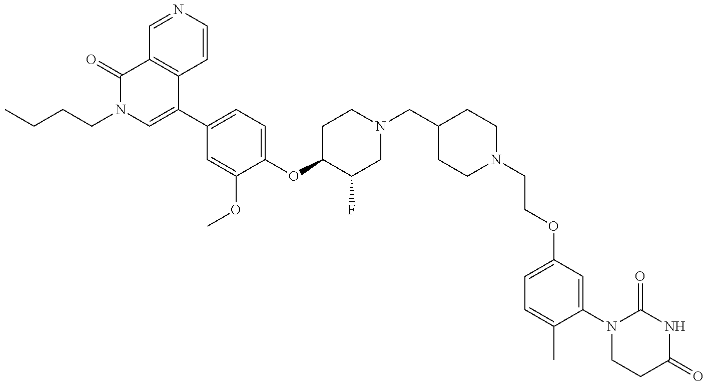 [SMILES: CCCCn1cc(-c2ccc(O[C@H]3CCN(CC4CCN(CCOc5ccc(C)c(N6CCC(=O)NC6=O)c5)CC4)C[C@@H]3F)c(OC)c2)c2ccncc2c1=O] |
| E25 | 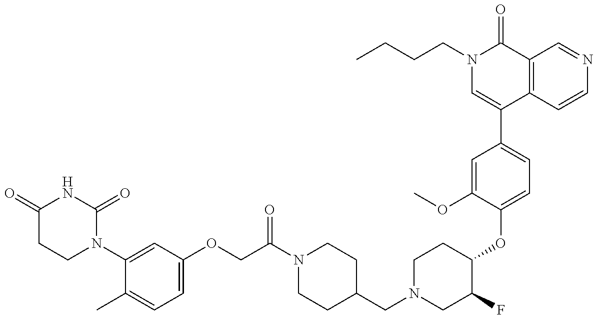 [SMILES: CCCCn1cc(-c2ccc(O[C@H]3CCN(CC4CCN(C(=O)COc5ccc(C)c(N6CCC(=O)NC6=O)c5)CC4)C[C@@H]3F)c(OC)c2)c2ccncc2c1=O] |
| E29 | 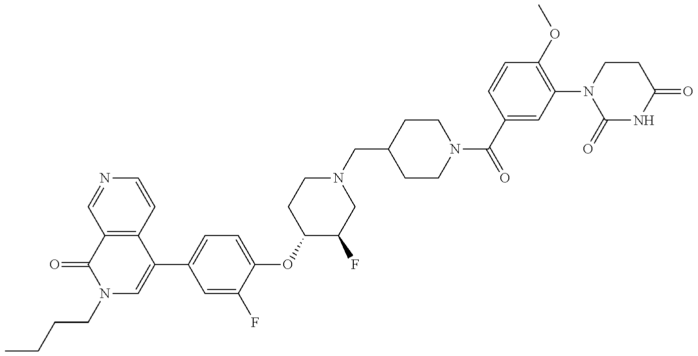 [SMILES: CCCCn1cc(-c2ccc(O[C@@H]3CCN(CC4CCN(C(=O)c5ccc(OC)c(N6CCC(=O)NC6=O)c5)CC4)C[C@H]3F)c(F)c2)c2ccncc2c1=O] |
| E31 | 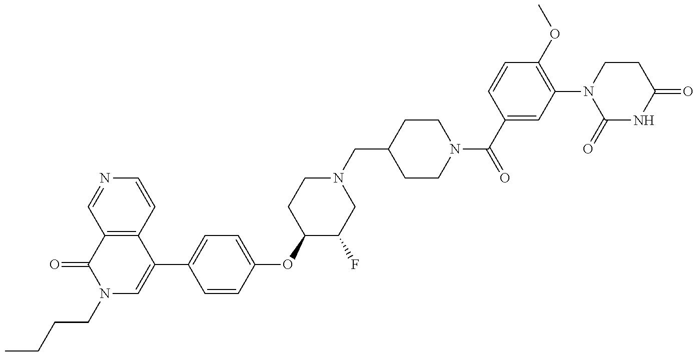 [SMILES: CCCCn1cc(-c2ccc(O[C@H]3CCN(CC4CCN(C(=O)c5ccc(OC)c(N6CCC(=O)NC6=O)c5)CC4)C[C@@H]3F)cc2)c2ccncc2c1=O] |
| E32 | 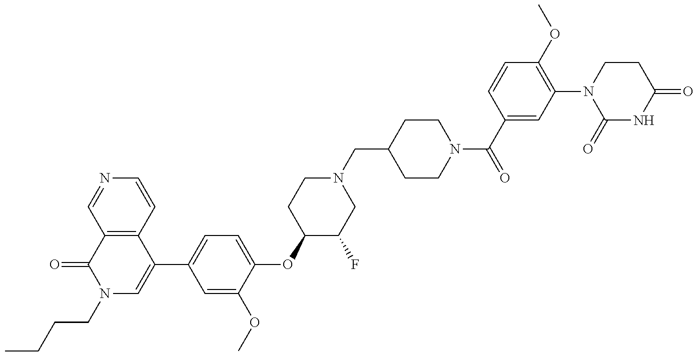 [SMILES: CCCCn1cc(-c2ccc(O[C@H]3CCN(CC4CCN(C(=O)c5ccc(OC)c(N6CCC(=O)NC6=O)c5)CC4)C[C@@H]3F)c(OC)c2)c2ccncc2c1=O] |
| E33 | 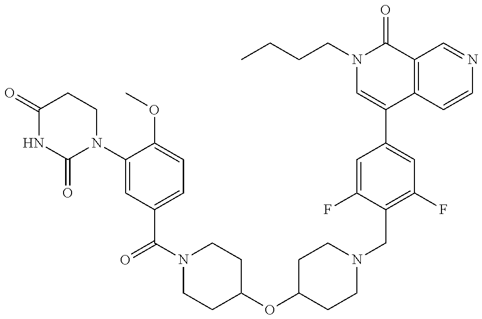 [SMILES: CCCCn1cc(-c2cc(F)c(CN3CCC(OC4CCN(C(=O)c5ccc(OC)c(N6CCC(=O)NC6=O)c5)CC4)CC3)c(F)c2)c2ccncc2c1=O] |
| E34 | 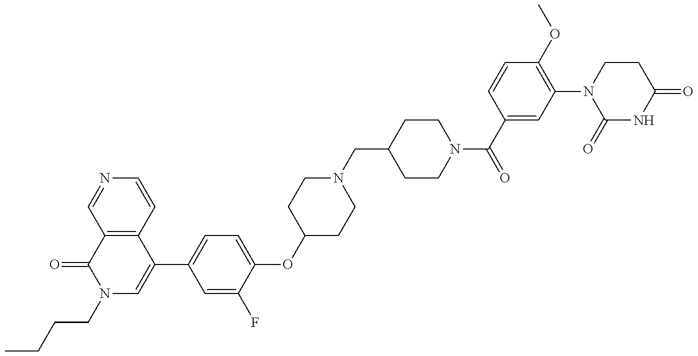 [SMILES: CCCCn1cc(-c2ccc(OC3CCN(CC4CCN(C(=O)c5ccc(OC)c(N6CCC(=O)NC6=O)c5)CC4)CC3)c(F)c2)c2ccncc2c1=O] |
| E36 | 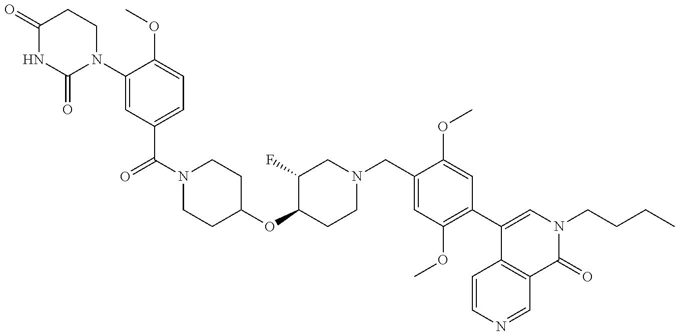 [SMILES: CCCCn1cc(-c2cc(OC)c(CN3CC[C@@H](OC4CCN(C(=O)c5ccc(OC)c(N6CCC(=O)NC6=O)c5)CC4)[C@H](F)C3)cc2OC)c2ccncc2c1=O] |
| E40 | 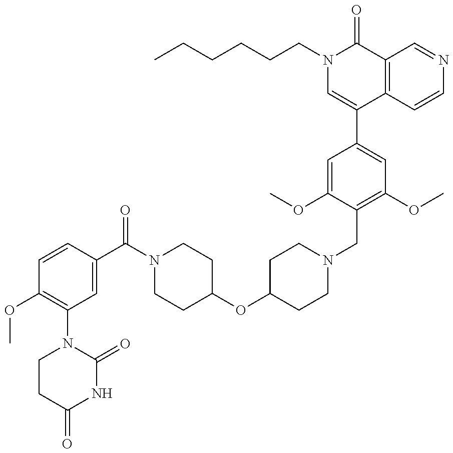 [SMILES: CCCCCCn1cc(-c2cc(OC)c(CN3CCC(OC4CCN(C(=O)c5ccc(OC)c(N6CCC(=O)NC6=O)c5)CC4)CC3)c(OC)c2)c2ccncc2c1=O] |
| E42 | 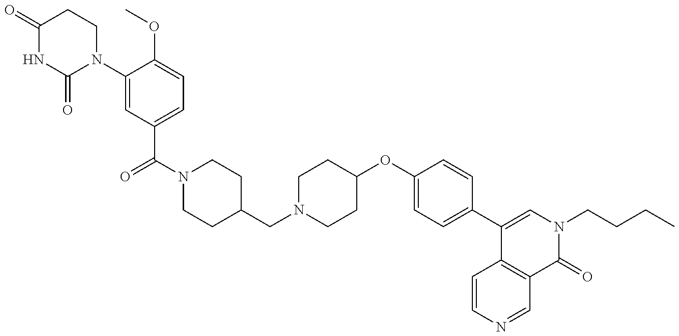 [SMILES: CCCCn1cc(-c2ccc(OC3CCN(CC4CCN(C(=O)c5ccc(OC)c(N6CCC(=O)NC6=O)c5)CC4)CC3)cc2)c2ccncc2c1=O] |
| E43 | 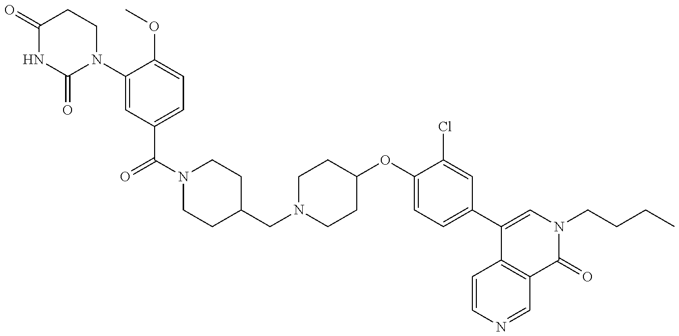 [SMILES: CCCCn1cc(-c2ccc(OC3CCN(CC4CCN(C(=O)c5ccc(OC)c(N6CCC(=O)NC6=O)c5)CC4)CC3)c(Cl)c2)c2ccncc2c1=O] |
| E45 | 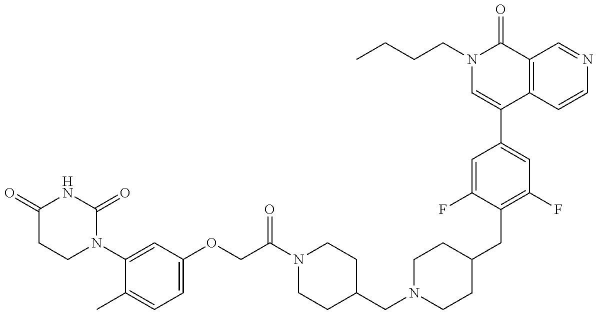 [SMILES: CCCCn1cc(-c2cc(F)c(CC3CCN(CC4CCN(C(=O)COc5ccc(C)c(N6CCC(=O)NC6=O)c5)CC4)CC3)c(F)c2)c2ccncc2c1=O] |
| E46 | 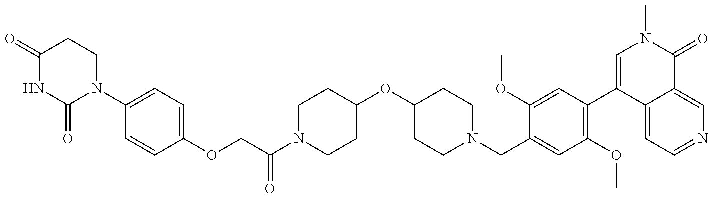 [SMILES: COc1cc(-c2cn(C)c(=O)c3cnccc23)c(OC)cc1CN1CCC(OC2CCN(C(=O)COc3ccc(N4CCC(=O)NC4=O)cc3)CC2)CC1] |
| E47 | 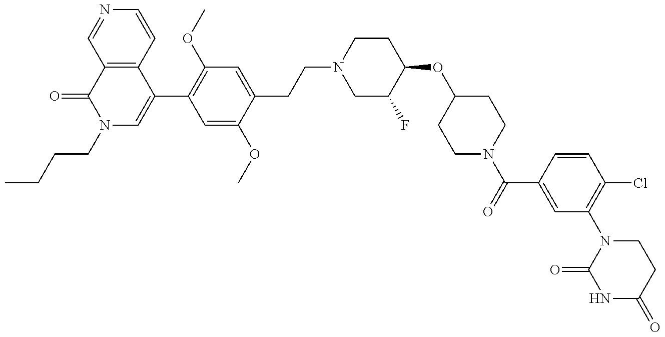 [SMILES: CCCCn1cc(-c2cc(OC)c(CCN3CC[C@@H](OC4CCN(C(=O)c5ccc(Cl)c(N6CCC(=O)NC6=O)c5)CC4)[C@H](F)C3)cc2OC)c2ccncc2c1=O] |
| E48 | 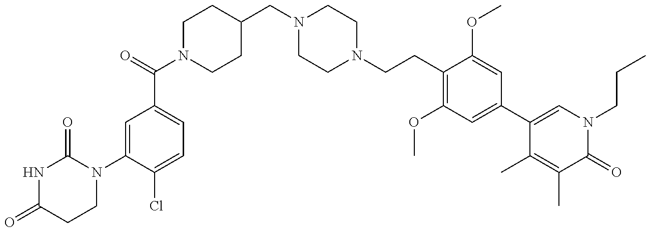 [SMILES: CCCn1cc(-c2cc(OC)c(CCN3CCN(CC4CCN(C(=O)c5ccc(Cl)c(N6CCC(=O)NC6=O)c5)CC4)CC3)c(OC)c2)c(C)c(C)c1=O] |
| E49 | 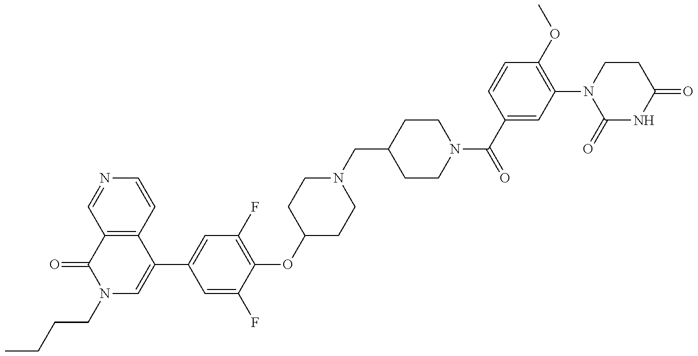 [SMILES: CCCCn1cc(-c2cc(F)c(OC3CCN(CC4CCN(C(=O)c5ccc(OC)c(N6CCC(=O)NC6=O)c5)CC4)CC3)c(F)c2)c2ccncc2c1=O] |
| E50 | 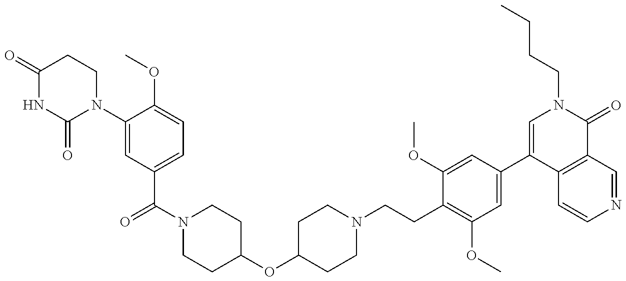 [SMILES: CCCCn1cc(-c2cc(OC)c(CCN3CCC(OC4CCN(C(=O)c5ccc(OC)c(N6CCC(=O)NC6=O)c5)CC4)CC3)c(OC)c2)c2ccncc2c1=O] |
| E51 | 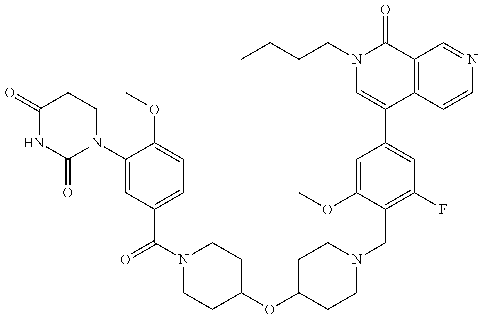 [SMILES: CCCCn1cc(-c2cc(F)c(CN3CCC(OC4CCN(C(=O)c5ccc(OC)c(N6CCC(=O)NC6=O)c5)CC4)CC3)c(OC)c2)c2ccncc2c1=O] |
| E52 | 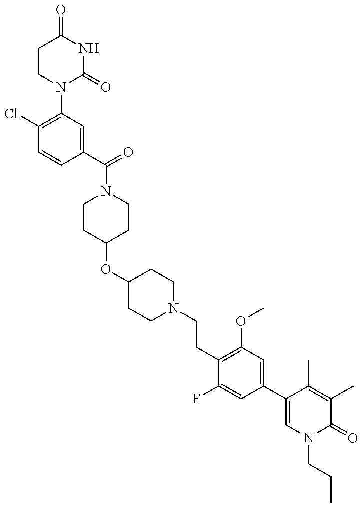 [SMILES: CCCn1cc(-c2cc(F)c(CCN3CCC(OC4CCN(C(=O)c5ccc(Cl)c(N6CCC(=O)NC6=O)c5)CC4)CC3)c(OC)c2)c(C)c(C)c1=O] |
| E53 | 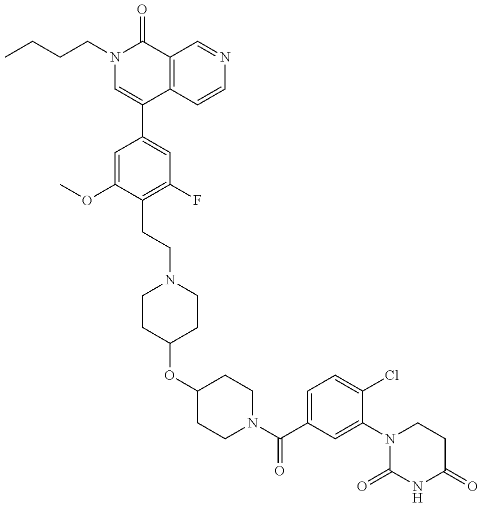 [SMILES: CCCCn1cc(-c2cc(F)c(CCN3CCC(OC4CCN(C(=O)c5ccc(Cl)c(N6CCC(=O)NC6=O)c5)CC4)CC3)c(OC)c2)c2ccncc2c1=O] |
| E54 | 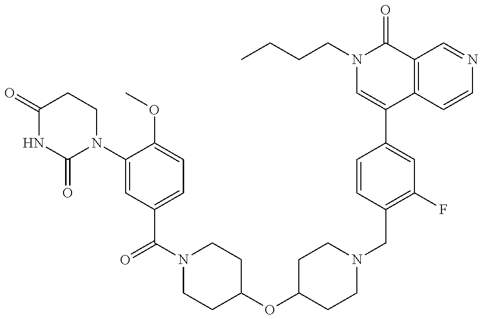 [SMILES: CCCCn1cc(-c2ccc(CN3CCC(OC4CCN(C(=O)c5ccc(OC)c(N6CCC(=O)NC6=O)c5)CC4)CC3)c(F)c2)c2ccncc2c1=O] |
| E55 | 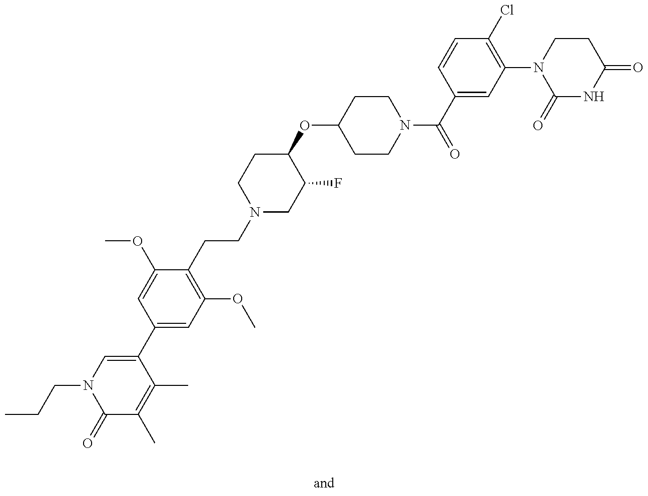 [SMILES: CCCn1cc(-c2cc(OC)c(CCN3CC[C@@H](OC4CCN(C(=O)c5ccc(Cl)c(N6CCC(=O)NC6=O)c5)CC4)[C@H](F)C3)c(OC)c2)c(C)c(C)c1=O] |
| E56 | 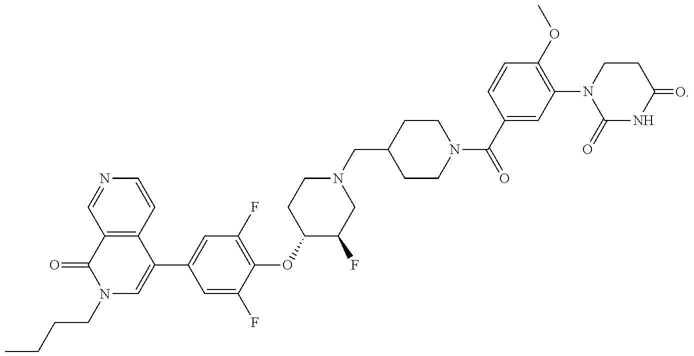 [SMILES: CCCCn1cc(-c2cc(F)c(O[C@@H]3CCN(CC4CCN(C(=O)c5ccc(OC)c(N6CCC(=O)NC6=O)c5)CC4)C[C@H]3F)c(F)c2)c2ccncc2c1=O] |

17. The compound of any one of claims 1-3, 6, 7, and 9-15, or a pharmaceutically acceptable salt, hydrate, solvate, prodrug, stereoisomer, or tautomer thereof, wherein the compound is a compound of Formula (BF-III) or a pharmaceutically acceptable salt, hydrate, solvate, prodrug, stereoisomer, or tautomer thereof,
wherein
R¹ and R² are methyl; or R¹ and R² together with the atoms to which they are attached form a pyridyl;
R³ is selected from the group consisting of methoxy, chloro, and fluoro;
L¹ is selected from the group consisting of a O and C₁₋₃ alkylene;
X¹-L²-X² is selected from the group consisting of: 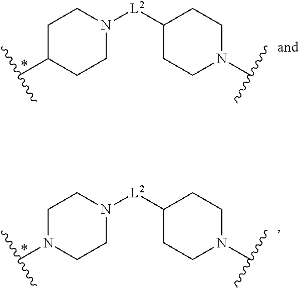
wherein * denotes the point of attachment to L¹, and wherein the heterocyclyl is substituted with 0 or 1 occurrences of R^{a}, wherein each R^{a} is fluoro;
L² is selected from the group consisting of C₁₋₃ alkylene and O;
L³ is selected from the group consisting of a C(O) and C₁₋₃ heteroalkylene;
R⁴ is selected from the group consisting of methyl, methoxy, chloro, and fluoro;
R⁵ is C₃₋₆ alkyl; and
m and n are each independently 1 or 2.

18. The compound of any one of claims 1-3, 6, 7, and 9-17, or a pharmaceutically acceptable salt, hydrate, solvate, prodrug, stereoisomer, or tautomer thereof, selected from the group consisting of:

| Compound | |
|---|---|
| Number | Compound Structure |
| E22 | 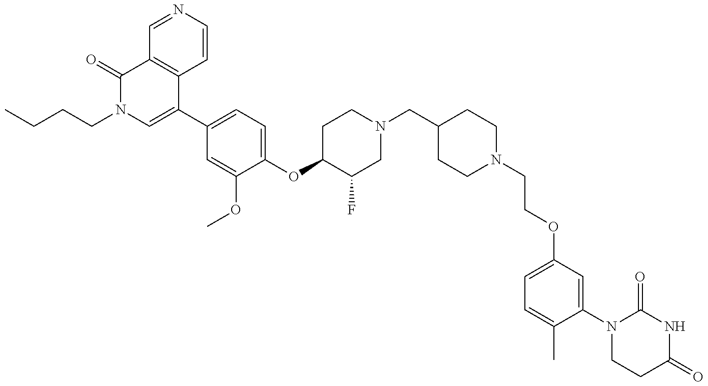 [SMILES: CCCCn1cc(-c2ccc(O[C@H]3CCN(CC4CCN(CCOc5ccc(C)c(N6CCC(=O)NC6=O)c5)CC4)C[C@@H]3F)c(OC)c2)c2ccncc2c1=O] |
| E29 | 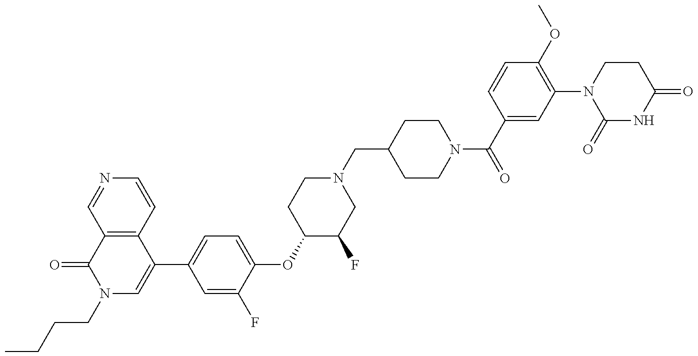 [SMILES: CCCCn1cc(-c2ccc(O[C@@H]3CCN(CC4CCN(C(=O)c5ccc(OC)c(N6CCC(=O)NC6=O)c5)CC4)C[C@H]3F)c(F)c2)c2ccncc2c1=O] |
| E48 | 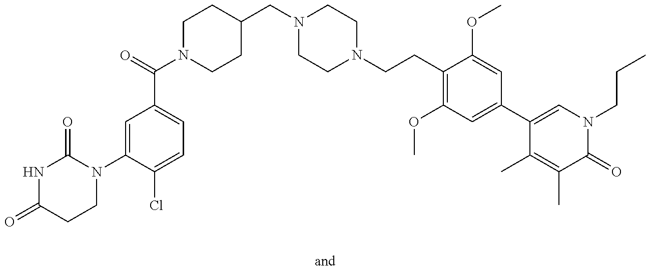 [SMILES: CCCn1cc(-c2cc(OC)c(CCN3CCN(CC4CCN(C(=O)c5ccc(Cl)c(N6CCC(=O)NC6=O)c5)CC4)CC3)c(OC)c2)c(C)c(C)c1=O] |
| E56 | 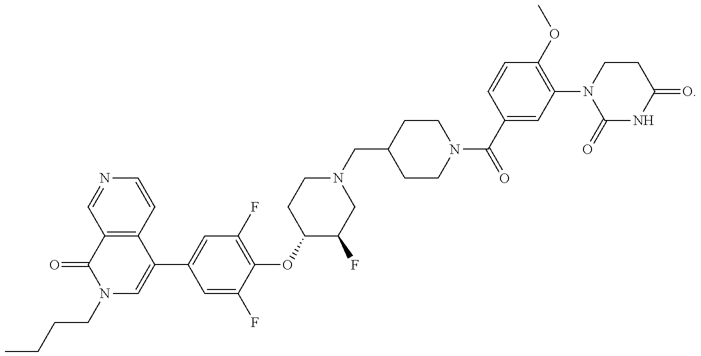 [SMILES: CCCCn1cc(-c2cc(F)c(O[C@@H]3CCN(CC4CCN(C(=O)c5ccc(OC)c(N6CCC(=O)NC6=O)c5)CC4)C[C@H]3F)c(F)c2)c2ccncc2c1=O] |

19. The compound of any one of claims 1, 4-6, and 8-12, wherein the compound is a compound of Formula (BF-II′): 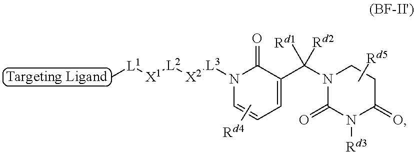
or a pharmaceutically acceptable salt, hydrate, solvate, prodrug, stereoisomer, or tautomer thereof, wherein:
L¹ is selected from the group consisting of a bond, O, NR′, C(O), C₁₋₆ alkylene, C₁₋₆ heteroalkylene, *C(O)—C₁₋₆ alkylene, and *C(O)—C₁₋₆ heteroalkylene, wherein * denotes the point of attachment of L¹ to the Targeting Ligand in Formula (BF-II′);
X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;
L² is selected from the group consisting of a bond, O, NR′, C₁₋₆ alkylene, and C₁₋₆ heteroalkylene; or
X¹-L²-X² form a spiroheterocyclyl;
L³ is selected from the group consisting of a bond, C₁₋₆ alkylene, C₁₋₆ heteroalkylene, *C(O)—C₁₋₆ alkylene, and *C(O)—C₁₋₆ heteroalkylene, wherein * denotes the point of attachment of L³ to X² in Formula (BF-II′); wherein no more than 2 of L¹, X¹, X², L², and L³ can simultaneously be a bond;
R′ is selected from the group consisting of hydrogen and C₁₋₆ alkyl;
R^{d1} and R^{d2} are each independently selected from the group consisting of H, C₁₋₆ alkyl, C₁₋₆ alkoxyl, C₁₋₆ haloalkyl, and C₁₋₆ heteroalkyl;
R^{d3} is H;
R^{d4} is selected from the group consisting of H, C₁₋₆ alkyl, halo, C₁₋₆ haloalkyl, and C₁₋₆ heteroalkyl;
R^{d5} is selected from the group consisting of H, C₁₋₆ alkyl, halo, C₁₋₆ haloalkyl, and C₁₋₆ heteroalkyl; and
the Targeting Ligand is a group that is capable of binding to a bromodomain-containing protein, e.g., BRD9.

20. The compound of any one of claims 1, 4-6, 8-12 and 19, wherein the compound is a compound of Formula (BF-III′): 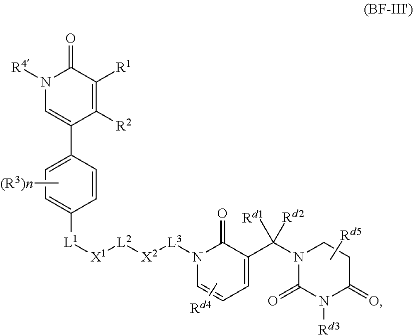
or a pharmaceutically acceptable salt, hydrate, solvate, prodrug, stereoisomer, or tautomer thereof, wherein:
R¹ and R² are independently selected from the group consisting of hydrogen and C₁₋₆ alkyl; or R¹ and R² together with the atoms to which they are attached form an aryl or heteroaryl;
R³ is selected from the group consisting of C₁₋₆ alkyl, C₁₋₆ alkoxyl, and halogen;
R′ is selected from the group consisting of hydrogen and C₁₋₆ alkyl;
L¹ is selected from the group consisting of a bond, O, NR′, C(O), C₁₋₆ alkylene, C₁₋₆ heteroalkylene, *C(O)—C₁₋₆ alkylene, and *C(O)—C₁₋₆ heteroalkylene, wherein * denotes the point of attachment of L¹ to the phenyl ring in Formula (BF-III′);
X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;
L² is selected from the group consisting of a bond, O, NR′, C₁₋₆ alkylene, and C₁₋₆ heteroalkylene; or
X¹-L²-X² form a spiroheterocyclyl;
L³ is selected from the group consisting of a bond, C₁₋₆ alkylene, C₁₋₆ heteroalkylene, *C(O)—C₁₋₆ alkylene, and *C(O)—C₁₋₆ heteroalkylene, wherein * denotes the point of attachment of L³ to X² in Formula (BF-III′); wherein no more than 2 of L¹, X¹, X², L², and L³ can simultaneously be a bond;
R′ is selected from the group consisting of hydrogen and C₁₋₆ alkyl;
n is 0, 1, or 2;
R^{d1} and R^{d2} are each independently selected from the group consisting of H, C₁₋₆ alkyl, C₁₋₆ alkoxyl, C₁₋₆ haloalkyl, and C₁₋₆ heteroalkyl;
R^{d3} is H;
R^{d4} is selected from the group consisting of H, C₁₋₆ alkyl, halo, C₁₋₆ haloalkyl, and C₁₋₆ heteroalkyl; and
R^{d5} is selected from the group consisting of H, C₁₋₆ alkyl, halo, C₁₋₆ haloalkyl, and C₁₋₆ heteroalkyl.

21. The compound of any one of claims 1, 4-6, 8-12, 19 and 20, or a pharmaceutically acceptable salt, hydrate, solvate, prodrug, stereoisomer, or tautomer thereof, wherein the compound is a compound of Formula (BF-III′) or a pharmaceutically acceptable salt, hydrate, solvate, prodrug, stereoisomer, or tautomer thereof,
wherein
R¹ and R² are independently selected from the group consisting of hydrogen and C₁₋₆ alkyl; or R¹ and R² together with the atoms to which they are attached form a heteroaryl;
R³ is selected from the group consisting of C₁₋₆ alkoxyl and halogen;
R^{4′} is C₁₋₆ alkyl;
L¹ is selected from the group consisting of O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, wherein * denotes the point of attachment of L¹ to the phenyl ring in Formula (BF-III′);
X¹-L²-X² is selected from the group consisting of: 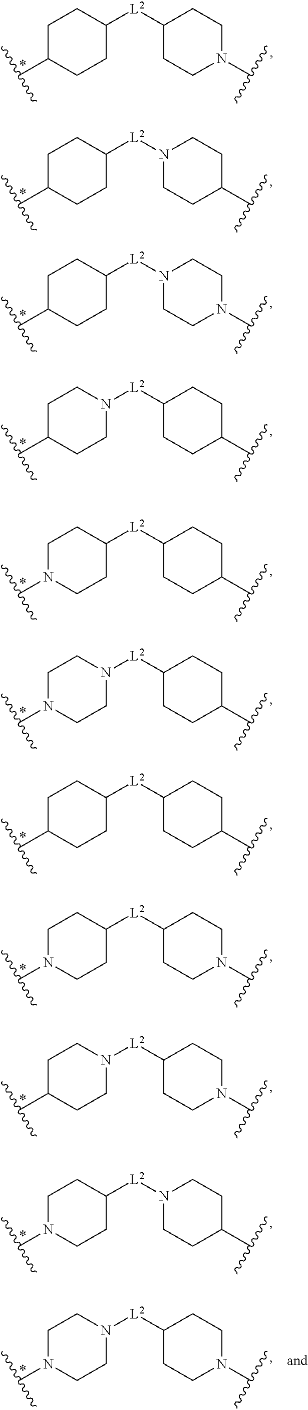 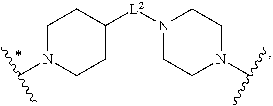
wherein * denotes the point of attachment to L¹, and wherein the carbocyclyl and heterocyclyl are substituted with 0-4 occurrences of R^{a}, wherein each R^{a} is halogen;
L² is selected from the group consisting of O, C₁₋₆ alkylene, and C₁₋₆ heteroalkylene; or
L³ is selected from the group consisting of C₁₋₆ alkylene and C₁₋₆ heteroalkylene, wherein * denotes the point of attachment of L³ to X² in Formula (BF-III′);
n is 0, 1, or 2;
R^{d1} and R^{d2} are each independently selected from the group consisting of H and C₁₋₆ alkyl;
R^{d3} is H;
R^{d4} is selected from the group consisting of H, C₁₋₆ alkyl, and halogen; and
R^{d5} is selected from the group consisting of H and C₁₋₆ alkyl.

22. The compound of any one of claims 1, 4-6, 8-12, and 19-21, the compound is a compound of Formula (BF-IV′): 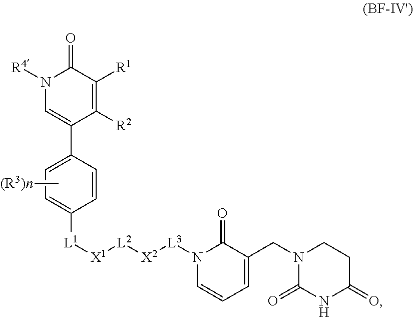
or a pharmaceutically acceptable salt, hydrate, solvate, prodrug, stereoisomer, or tautomer thereof, wherein:
R¹ and R² are independently selected from the group consisting of hydrogen and C₁₋₆ alkyl; or R¹ and R² together with the atoms to which they are attached form an aryl or heteroaryl;
R³ is selected from the group consisting of C₁₋₆ alkyl, C₁₋₆ alkoxyl, and halogen;
R^{4′} is selected from the group consisting of hydrogen or C₁₋₆ alkyl;
L¹ is selected from the group consisting of a bond, O, NR′, C(O), C₁₋₆ alkylene, C₁₋₆ heteroalkylene, *C(O)—C₁₋₆ alkylene, and *C(O)—C₁₋₆ heteroalkylene, wherein * denotes the point of attachment of L¹ to the phenyl ring in Formula (BF-IV′);
X¹ and X² are each independently selected from the group consisting of a bond, carbocyclyl, and heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen;
L² is selected from the group consisting of a bond, O, NR′, C₁₋₆ alkylene, and C₁₋₆ heteroalkylene; or
X¹-L²-X² form a spiroheterocyclyl;
L³ is selected from the group consisting of a bond, C₁₋₆ alkylene, C₁₋₆ heteroalkylene, *C(O)—C₁₋₆ alkylene, and *C(O)—C₁₋₆ heteroalkylene, wherein * denotes the point of attachment of L³ to X² in Formula (BF-IV′); wherein no more than 2 of L¹, X¹, X², L², and L³ can simultaneously be a bond;
R′ is selected from the group consisting of hydrogen and C₁₋₆ alkyl; and
n is 0, 1, or 2.

23. The compound of any one of claims 1, 4-6, 8-12, and 19-22, or a pharmaceutically acceptable salt, hydrate, solvate, prodrug, stereoisomer, or tautomer thereof, wherein the compound is a compound of Formula (BF-IV′) or a pharmaceutically acceptable salt, hydrate, solvate, prodrug, stereoisomer, or tautomer thereof,
wherein
R¹ and R² are C₁₋₆ alkyl; or R¹ and R² together with the atoms to which they are attached form a heteroaryl;
R³ is selected from the group consisting of C₁₋₆ alkoxyl and halogen;
R^{4′} is C₁₋₆ alkyl;
L¹ is selected from the group consisting of O and C₁₋₆ alkylene;
X¹-L²-X² is selected from the group consisting of: 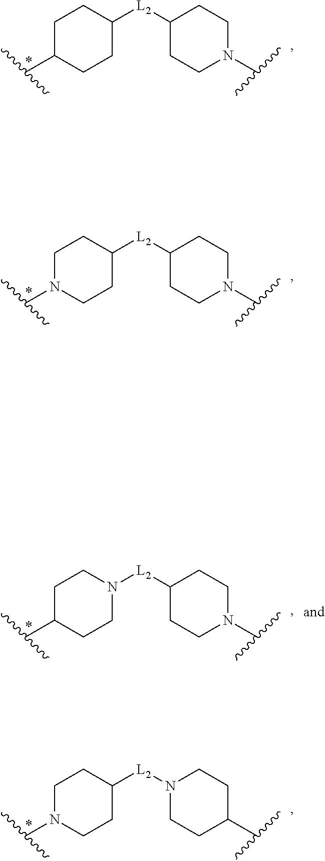
wherein * denotes the point of attachment to L¹, and wherein the carbocyclyl and heterocyclyl are substituted with 0-4 occurrences of R^{a}, wherein each R^{a} is halogen;
L² is selected from the group consisting of O and C₁₋₆ alkylene;
L³ is C₁₋₆ alkylene; and
n is 0, 1, or 2.

24. The compound of any one of claims 1, 4-6, 8-12, and 19-23, or a pharmaceutically acceptable salt, hydrate, solvate, prodrug, stereoisomer, or tautomer thereof, wherein the compound is a compound of Formula (BF-IV′) or a pharmaceutically acceptable salt, hydrate, solvate, prodrug, stereoisomer, or tautomer thereof,
wherein
R¹ and R² are methyl; or R¹ and R² together with the atoms to which they are attached form a pyridyl;
R³ is selected from the group consisting of methoxy, chloro, and fluoro;
R⁴ is C₁₋₆ alkyl;
L¹ is selected from the group consisting of O and C₁₋₃ alkylene;
X¹-L²-X² is selected from the group consisting of: 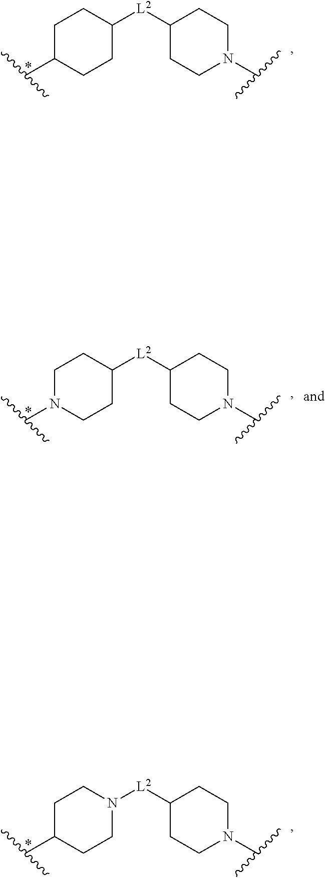
wherein * denotes the point of attachment to L¹, and wherein the carbocyclyl and heterocyclyl are substituted with 0-4 occurrences of R^{a}, wherein each R^{a} is fluoro;
L² is selected from the group consisting of O and C₁₋₃ alkylene;
L³ is C₂₋₃ alkylene;
n is 0, 1, or 2.

25. The compound of any one of claims 1, 4-6, 8-12, and 19-24, or a pharmaceutically acceptable salt, hydrate, solvate, prodrug, stereoisomer, or tautomer thereof, selected from the group consisting of:

| Compound | |
|---|---|
| Number | Compound Structure |
| C1 | 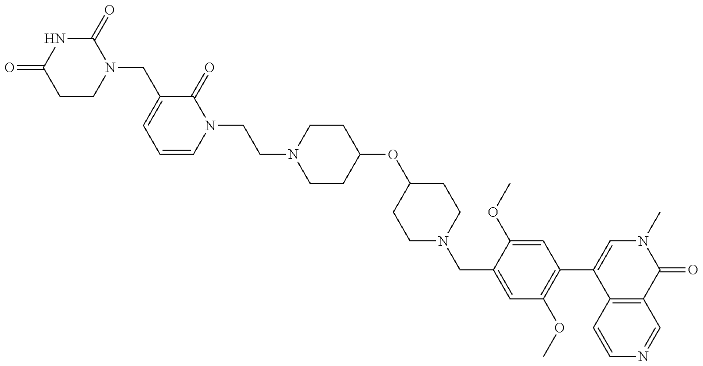 [SMILES: COc1cc(-c2cn(C)c(=O)c3cnccc23)c(OC)cc1CN1CCC(OC2CCN(CCn3cccc(CN4CCC(=O)NC4=O)c3=O)CC2)CC1] |
| C2 | 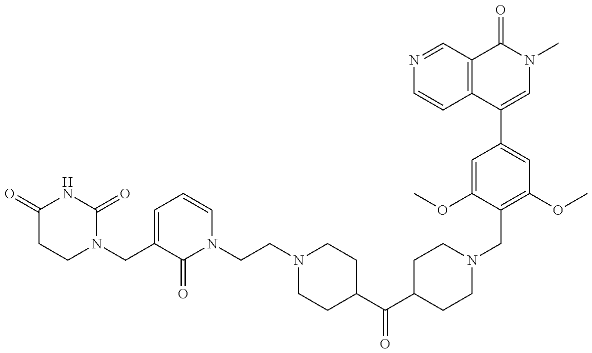 [SMILES: COc1cc(-c2cn(C)c(=O)c3cnccc23)cc(OC)c1CN1CCC(C(=O)C2CCN(CCn3cccc(CN4CCC(=O)NC4=O)c3=O)CC2)CC1] |
| C3 | 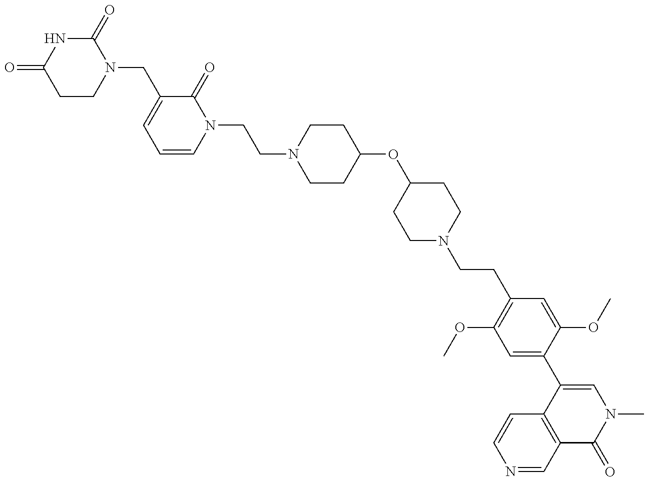 [SMILES: COc1cc(-c2cn(C)c(=O)c3cnccc23)c(OC)cc1CCN1CCC(OC2CCN(CCn3cccc(CN4CCC(=O)NC4=O)c3=O)CC2)CC1] |
| C4 | 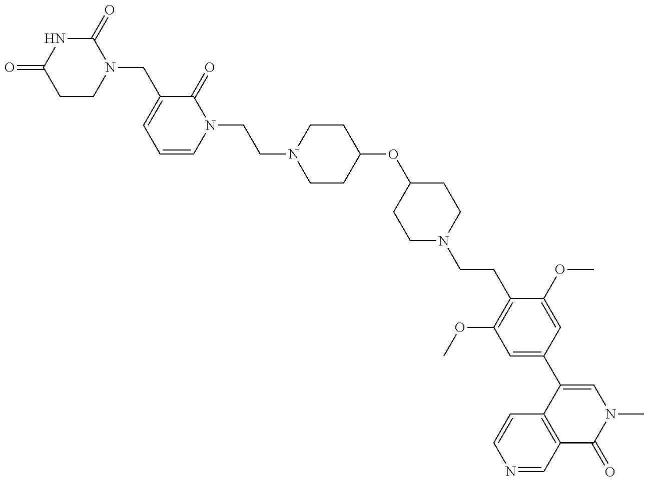 [SMILES: COc1cc(-c2cn(C)c(=O)c3cnccc23)cc(OC)c1CCN1CCC(OC2CCN(CCn3cccc(CN4CCC(=O)NC4=O)c3=O)CC2)CC1] |
| D1 | 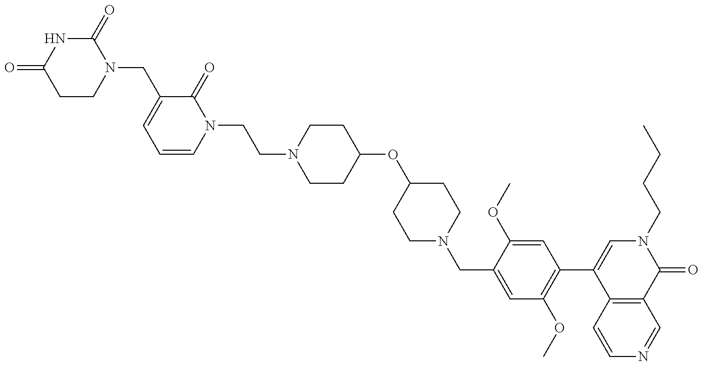 [SMILES: CCCCn1cc(-c2cc(OC)c(CN3CCC(OC4CCN(CCn5cccc(CN6CCC(=O)NC6=O)c5=O)CC4)CC3)cc2OC)c2ccncc2c1=O] |
| D2 | 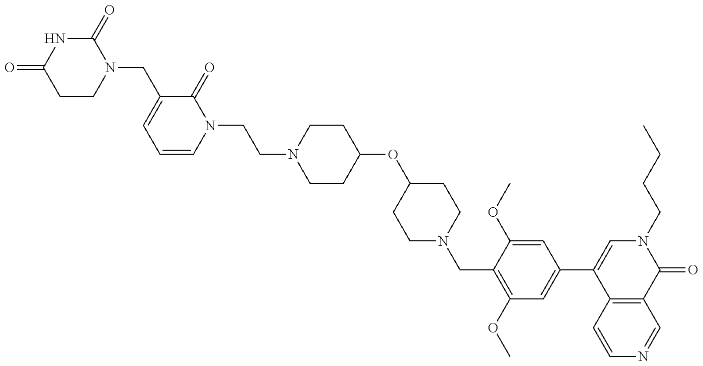 [SMILES: CCCCn1cc(-c2cc(OC)c(CN3CCC(OC4CCN(CCn5cccc(CN6CCC(=O)NC6=O)c5=O)CC4)CC3)c(OC)c2)c2ccncc2c1=O] |
| D3 | 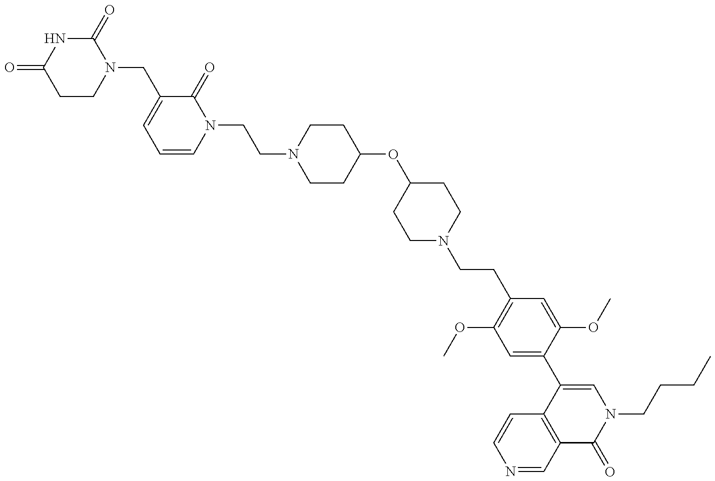 [SMILES: CCCCn1cc(-c2cc(OC)c(CCN3CCC(OC4CCN(CCn5cccc(CN6CCC(=O)NC6=O)c5=O)CC4)CC3)cc2OC)c2ccncc2c1=O] |
| D4 | 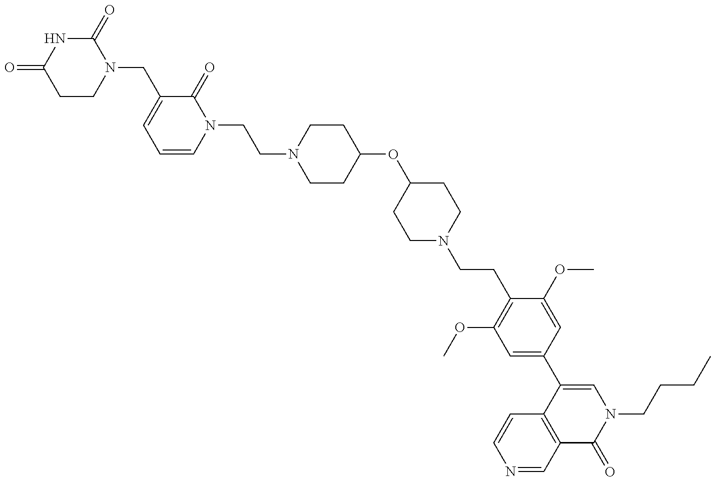 [SMILES: CCCCn1cc(-c2cc(OC)c(CCN3CCC(OC4CCN(CCn5cccc(CN6CCC(=O)NC6=O)c5=O)CC4)CC3)c(OC)c2)c2ccncc2c1=O] |
| E1 | 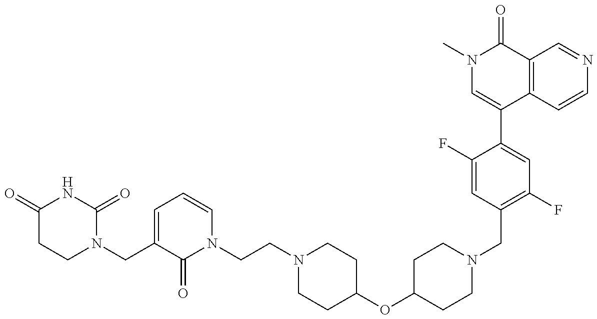 [SMILES: Cn1cc(-c2cc(F)c(CN3CCC(OC4CCN(CCn5cccc(CN6CCC(=O)NC6=O)c5=O)CC4)CC3)cc2F)c2ccncc2c1=O] |
| E2 | 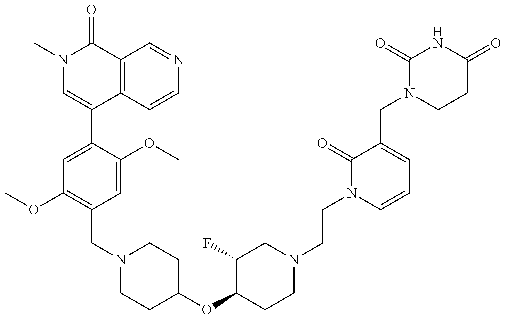 [SMILES: COc1cc(-c2cn(C)c(=O)c3cnccc23)c(OC)cc1CN1CCC(O[C@@H]2CCN(CCn3cccc(CN4CCC(=O)NC4=O)c3=O)C[C@H]2F)CC1] |
| E7 | 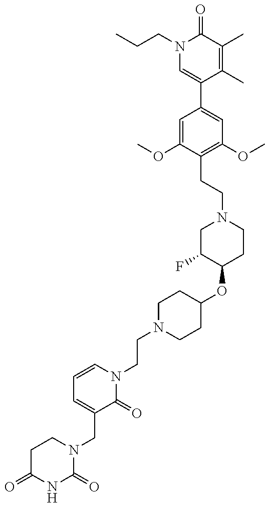 [SMILES: CCCn1cc(-c2cc(OC)c(CCN3CC[C@@H](OC4CCN(CCn5cccc(CN6CCC(=O)NC6=O)c5=O)CC4)[C@H](F)C3)c(OC)c2)c(C)c(C)c1=O] |
| E17 | 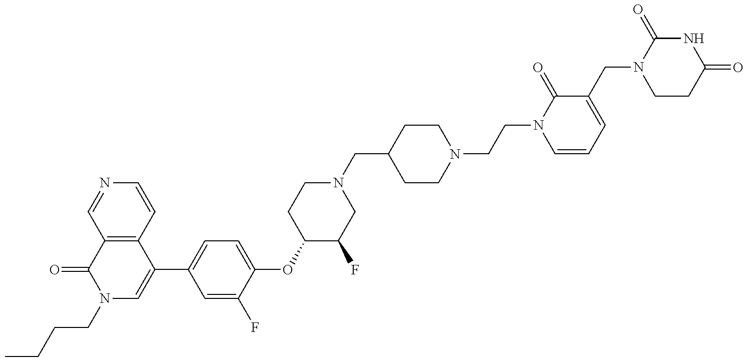 [SMILES: CCCCn1cc(-c2ccc(O[C@@H]3CCN(CC4CCN(CCn5cccc(CN6CCC(=O)NC6=O)c5=O)CC4)C[C@H]3F)c(F)c2)c2ccncc2c1=O] |
| E18 | 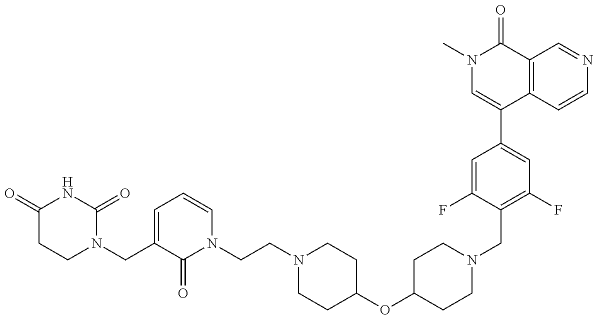 [SMILES: Cn1cc(-c2cc(F)c(CN3CCC(OC4CCN(CCn5cccc(CN6CCC(=O)NC6=O)c5=O)CC4)CC3)c(F)c2)c2ccncc2c1=O] |
| E23 | 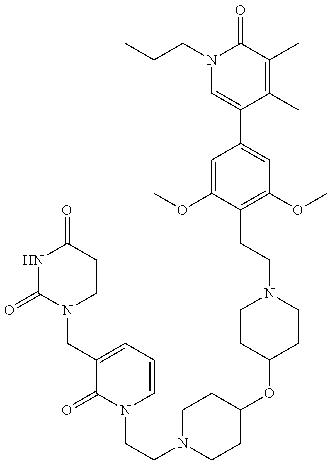 [SMILES: CCCn1cc(-c2cc(OC)c(CCN3CCC(OC4CCN(CCn5cccc(CN6CCC(=O)NC6=O)c5=O)CC4)CC3)c(OC)c2)c(C)c(C)c1=O] |
| E24 | 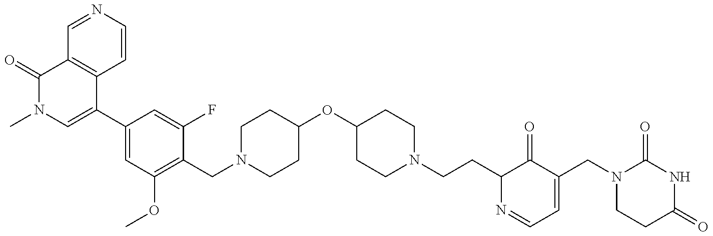 [SMILES: COc1cc(-c2cn(C)c(=O)c3cnccc23)cc(F)c1CN1CCC(OC2CCN(CCC3N=CC=C(CN4CCC(=O)NC4=O)C3=O)CC2)CC1] |
| E27 | 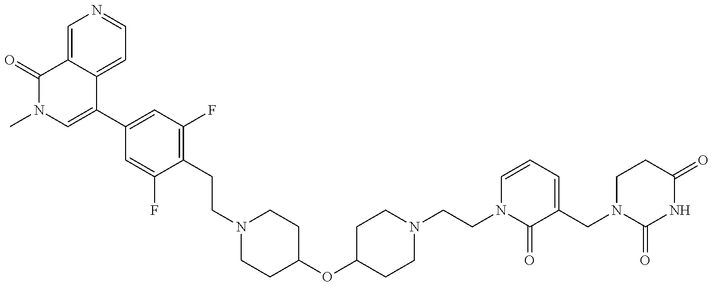 [SMILES: Cn1cc(-c2cc(F)c(CCN3CCC(OC4CCN(CCn5cccc(CN6CCC(=O)NC6=O)c5=O)CC4)CC3)c(F)c2)c2ccncc2c1=O] |
| E28 | 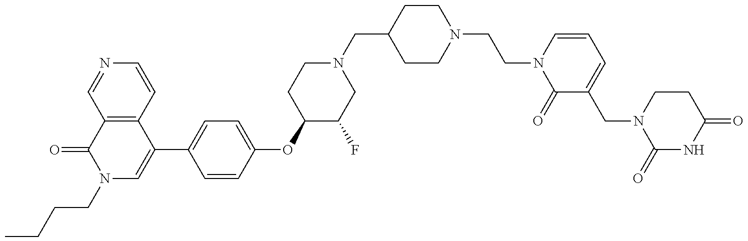 [SMILES: CCCCn1cc(-c2ccc(O[C@H]3CCN(CC4CCN(CCn5cccc(CN6CCC(=O)NC6=O)c5=O)CC4)C[C@@H]3F)cc2)c2ccncc2c1=O] |
| E30 | 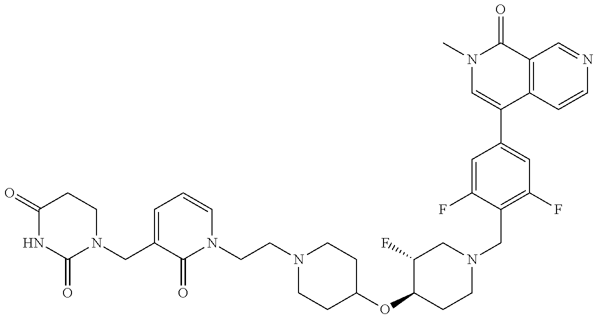 [SMILES: Cn1cc(-c2cc(F)c(CN3CC[C@@H](OC4CCN(CCn5cccc(CN6CCC(=O)NC6=O)c5=O)CC4)[C@H](F)C3)c(F)c2)c2ccncc2c1=O] |
| E35 | 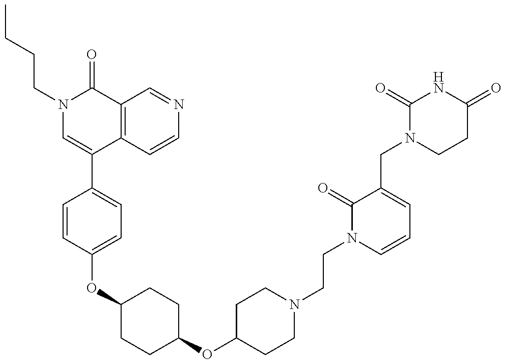 [SMILES: CCCCn1cc(-c2ccc(O[C@H]3CC[C@@H](OC4CCN(CCn5cccc(CN6CCC(=O)NC6=O)c5=O)CC4)CC3)cc2)c2ccncc2c1=O] |
| E37 | 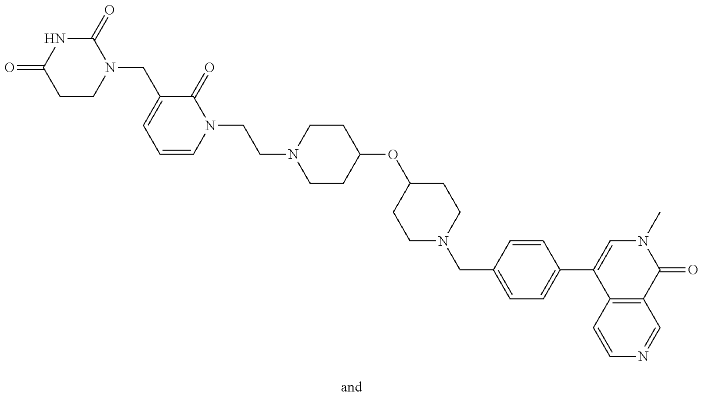 [SMILES: Cn1cc(-c2ccc(CN3CCC(OC4CCN(CCn5cccc(CN6CCC(=O)NC6=O)c5=O)CC4)CC3)cc2)c2ccncc2c1=O] |
| E39 | 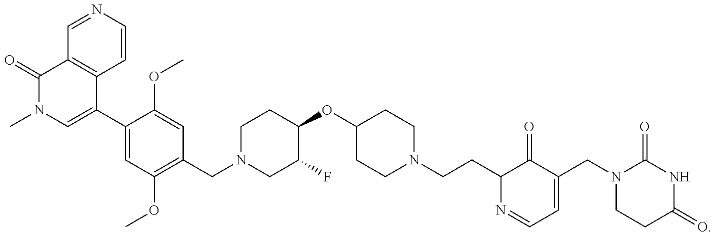 [SMILES: COc1cc(-c2cn(C)c(=O)c3cnccc23)c(OC)cc1CN1CC[C@@H](OC2CCN(CCC3N=CC=C(CN4CCC(=O)NC4=O)C3=O)CC2)[C@H](F)C1] |

26. A pharmaceutical composition comprising the compound of any one of the preceding claims or a pharmaceutically acceptable salt, hydrate, solvate, prodrug, stereoisomer, or tautomer thereof, and a pharmaceutically acceptable carrier.

27. A method of inhibiting or modulating a bromodomain-containing protein 9 (BRD9) in a subject in need thereof, the method comprising administering to the subject a therapeutically effective amount of the compound of any one of claims 1-25, or a pharmaceutically acceptable salt, hydrate, solvate, prodrug, stereoisomer, or tautomer thereof.

28. A method of treating cancer in a subject in need thereof, the method comprising administering to the subject a therapeutically effective amount of the compound of any one of claims 1-25, or a pharmaceutically acceptable salt, hydrate, solvate, prodrug, stereoisomer, or tautomer thereof.

29. The method of claim 28, wherein the cancer is selected from colorectal cancer, ovarian cancer, pancreatic cancer, renal cell carcinoma, hepatocellular carcinoma, bladder cancer, gastric cancer, breast cancer, glioma, medulloblastoma, squamous cell carcinoma, melanoma, lung, acute myeloid leukemia, synovial sarcoma, chronic lymphocytic leukemia, diffuse large B-cell lymphoma, non-Hodgkin lymphoma, Burkitt lymphoma, multiple myeloma, T-lineage acute lymphoblastic leukemia, clear-cell ovarian cancer, adenoid cystic carcinoma and malignant rhabdoid tumor.

30. A compound of any one of claims 1-25, or a pharmaceutically acceptable salt, hydrate, solvate, prodrug, stereoisomer, or tautomer thereof, for use in the treatment of a disease or disorder responsive to inhibiting or modulating BRD9.

31. A compound of any one of claims 1-25, or a pharmaceutically acceptable salt, hydrate, solvate, prodrug, stereoisomer, or tautomer thereof, for use in the treatment of cancer.

32. A compound of any one of claims 1-25, or a pharmaceutically acceptable salt, hydrate, solvate, prodrug, stereoisomer, or tautomer thereof, for use in the treatment of cancer, wherein the cancer is selected from colorectal cancer, ovarian cancer, pancreatic cancer, renal cell carcinoma, hepatocellular carcinoma, bladder cancer, gastric cancer, breast cancer, glioma, medulloblastoma, squamous cell carcinoma, melanoma, lung, acute myeloid leukemia, synovial sarcoma, chronic lymphocytic leukemia, diffuse large B-cell lymphoma, non-Hodgkin lymphoma, Burkitt lymphoma, multiple myeloma, T-lineage acute lymphoblastic leukemia, clear-cell ovarian cancer, adenoid cystic carcinoma and malignant rhabdoid tumor.


SEQUENCE LISTING (1 sequence)

SEQ ID NO: 1 - DNA, 1551 bp, Artificial Sequence
  source: /note=“Description of Artificial Sequence Synthetic polynucleotide”
gatatccagcacagtggcggccgctcgagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagcgacgaattttagtctactgaaacaagcgggagacgtggaggaaaaccctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagctcgagtctagagggcccgcggttaac